(19) United States
(12) Patent Application Publication
(10) Pub. No.: US 2023/0097157 A1
(43) Pub. Date: Mar. 30, 2023

(54) Bicyclic Pyridin-2-one and pyrimidin-4-one Derivatives Useful As a Factor XIa Inhibitors

(71) Applicant: Janssen Pharmaceutica NV, Beerse (BE)

(72) Inventors: Guozhang Xu, Chesterbrook, PA (US); Mark J. Macielag, Gwynedd Valley, PA (US); Micheal D. Gaul, Apex, NC (US); Tho V. Thieu, North Wales, PA (US); Zhijie Liu, Paoli, PA (US)

(21) Appl. No.: 17/870,156

(22) Filed: Jul. 21, 2022

Related U.S. Application Data
(60) Provisional application No. 63/224,429, filed on Jul. 22, 2021.

(51) Int. Cl. C07D 471/04

(52) U.S. Cl. CPC C07D 471/04

(57) ABSTRACT

The present invention is directed to bicyclic pyridinon-2-one and pyrimidin-4-one derivatives, stereoisomers, isotopologues, and pharmaceutically acceptable salts thereof, pharmaceutical compositions containing said compounds and the use of said compounds in the treatment and/or prophylaxis of thromboembolic disorders, inflammatory disorders and diseases or conditions in which plasma kallikrein activity is implicated.

### FIELD OF THE INVENTION

The present invention is directed bicyclic pyridinon-2-one and pyrimidin-4-one derivatives, stereoisomers, isotopologues, and pharmaceutically acceptable salts thereof, pharmaceutical compositions containing said compounds, and the use of said compounds in the treatment and/or prophylaxis of thromboembolic disorders, inflammatory disorders and diseases or conditions in which plasma kallikrein activity is implicated.

### BACKGROUND OF THE INVENTION

Thromboembolic diseases remain the leading cause of death in developed countries despite the availability of anticoagulants such as warfarin (COUMADIN®), heparin, low molecular weight heparins (LMWH), and synthetic penta-saccharides and antiplatelet agents such as aspirin and clopidogrel (PLAVIX®). The oral anticoagulant warfarin inhibits the post-translational maturation of coagulation factors VII, IX, X and prothrombin, and has proven effective in both venous and arterial thrombosis. However, its usage is limited due to its narrow therapeutic index with respect to bleeding safety, slow onset of therapeutic effect, numerous dietary and drug-drug interactions, and a need for monitoring and dose adjustment. Novel oral anticoagulants directly targeting either thrombin or factor Xa, e.g., dabigatran, apixaban, betrixaban, edoxaban, rivaroxaban, have been approved for both venous and arterial indications. However, the risk of bleeding is not completely eliminated, and can be as high as 2-3% per year in patients with atrial fibrillation (Quan et al., J. Med. Chem. 2018, pp 7425-7447, Vol. 61). Thus, discovering and developing safe and efficacious oral anticoagulants with minimal impacts on hemostasis for the prevention and treatment of a wide range of thromboembolic disorders has become increasingly important.

### SUMMARY OF THE INVENTION

The present invention is directed to compounds of formula (A)

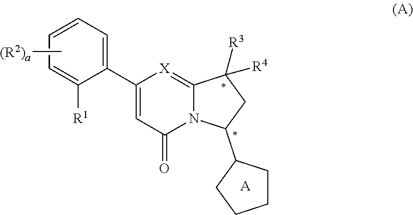

wherein

X is selected from the group consisting of CH and N;

R¹ is selected from the group consisting of halogen, hydroxy, C₁₋₄alkyl, fluorinated C₁₋₄alkyl, C₁₋₄alkoxy, fluorinated C₁₋₄alkoxy, cyano, nitro, —NR^{A}R^{B}, —C(O)—C₁₋₄alkyl, C₃₋₆cycloalkyl, phenyl and 5 to 6 membered heterocyclyl;

wherein the C₃₋₆cycloalkyl, phenyl or 5 to 6 membered heterocyclyl is optionally substituted with one or more substituents independently selected from the group consisting of halogen, hydroxy, cyano, C₁₋₄alkyl, fluorinated C₁₋₄alkyl, C₁₋₄alkoxy, fluorinated C₁₋₄alkoxy, —C(O)OH, —C(O)O—(C₁₋₄alkyl), —NR^{A}R^{B}, —(C₁₋₄alkyl)-NR^{A}R^{B}, C₃₋₇cycloalkyl and 5 to 6 membered heterocyclyl; and wherein R^{A} and R^{B} are each independently selected from the group consisting of hydrogen and C₁₋₄alkyl;

a is an integer from 1 to 3;

each R² is independently selected from the group consisting of chloro, fluoro, methyl and methoxy;

R³ is hydrogen; and R⁴ is selected from the group consisting of methyl, methoxy, and ethoxy;

alternatively, R³ and R⁴ are taken together with the carbon atom to which they are bound to form cyclopropyl;

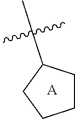

is selected from the group consisting of

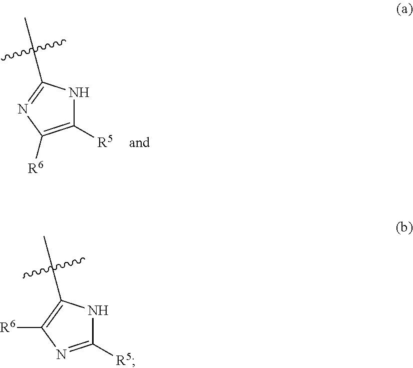

wherein R⁵ is selected from the group consisting of aryl and heterocyclyl;

wherein the aryl or heterocyclyl is optionally substituted with one or more substituents independently selected from the group consisting of halogen, hydroxy, oxo, C₁₋₄alkyl, fluorinated C₁₋₄alkyl, hydroxy substituted C₁₋₄alkyl, hydroxy substituted fluorinated C₁₋₄alkyl, C₁₋₄alkoxy, fluorinated C₁₋₄alkoxy, hydroxy substituted C₁₋₄alkoxy, hydroxy substituted fluorinated C₁₋₄alkoxy, cyano, —NR^{C}R^{D}, —(C₁₋₂alkylene)-NR^{C}R^{D}, —NR^{C}—C(O)—(C₁₋₄alkyl), —NR^{C}—C(O)—O—(C₁₋₄alkyl), —C(O)—NR^{C}—(C₁₋₄alkyl), —NR^{C}—SO₂—(C₁₋₄alkyl), cycloprop-1-yl, 1-hydroxy-cycloprop-1-yl, and —NR^{C}—C(O)-cyclopropyl; wherein R^{C} and R^{D} are each independently selected from the group consisting of hydrogen and C₁₋₄alkyl;

and R⁶ is selected from the group consisting of hydrogen, and halogen;

and tautomers, stereoisomers, isotopologues, and pharmaceutically acceptable salts thereof.

The present invention further is directed to compounds of formula (I)

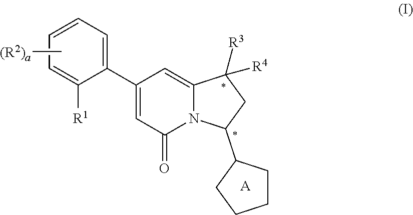

wherein

R¹ is selected from the group consisting of halogen, hydroxy, C₁₋₄alkyl, fluorinated C₁₋₄alkyl, C₁₋₄alkoxy, fluorinated C₁₋₄alkoxy, cyano, nitro, —NR^{A}R^{B}, —C(O)—C₁₋₄alkyl, C₃₋₆cycloalkyl, phenyl and 5 to 6 membered heterocyclyl;

wherein the C₃₋₆cycloalkyl, phenyl or 5 to 6 membered heterocyclyl is optionally substituted with one or more substituents independently selected from the group consisting of halogen, hydroxy, cyano, C₁₋₄alkyl, fluorinated C₁₋₄alkyl, C₁₋₄alkoxy, fluorinated C₁₋₄alkoxy, —C(O)OH, —C(O)O—(C₁₋₄alkyl), —NR^{A}R^{B}, —(C₁₋₄alkyl)-NR^{A}R^{B}, C₃₋₇cycloalkyl and 5 to 6 membered heterocyclyl;

and wherein R^{A} and R^{B} are each independently selected from the group consisting of hydrogen and C₁₋₄alkyl;

a is an integer from 1 to 3;

each R² is independently selected from the group consisting of chloro, fluoro, methyl and methoxy;

R³ is hydrogen; and R⁴ is selected from the group consisting of methyl, methoxy, and ethoxy;

alternatively, R³ and R⁴ are taken together with the carbon atom to which they are bound to form cyclopropyl;

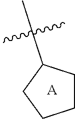

is selected from the group consisting of

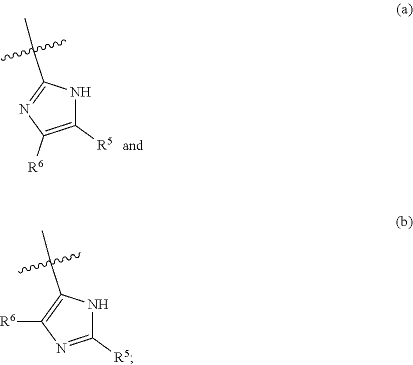

wherein R⁵ is selected from the group consisting of aryl and heterocyclyl;

wherein the aryl or heterocyclyl is optionally substituted with one or more substituents independently selected from the group consisting of halogen, hydroxy, oxo, C₁₋₄alkyl, fluorinated C₁₋₄alkyl, hydroxy substituted C₁₋₄alkyl, hydroxy substituted fluorinated C₁₋₄alkyl, C₁₋₄alkoxy, fluorinated C₁₋₄alkoxy, hydroxy substituted C₁₋₄alkoxy, hydroxy substituted fluorinated C₁₋₄alkoxy, cyano, —NR^{C}R^{D}, —(C₁₋₂alkylene)-NR^{C}R^{D}, —NR^{C}—C(O)—(C₁₋₄alkyl), —NR^{C}—C(O)—O—(C₁₋₄alkyl), —C(O)—NR^{C}—(C₁₋₄alkyl), —NR^{C}—SO₂—(C₁₋₄alkyl), cycloprop-1-yl, 1-hydroxy-cycloprop-1-yl, and —NR^{C}—C(O)-cyclopropyl; wherein R^{C} and R^{D} are each independently selected from the group consisting of hydrogen and C₁₋₄alkyl;

and tautomers, stereoisomers, isotopologues, and pharmaceutically acceptable salts thereof.

The present invention is further directed to compounds of formula (II)

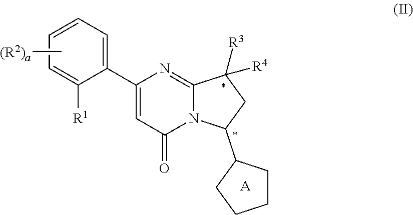

wherein

R¹ is selected from the group consisting of halogen, hydroxy, C₁₋₄alkyl, fluorinated C₁₋₄alkyl, C₁₋₄alkoxy, fluorinated C₁₋₄alkoxy, cyano, nitro, —NR^{A}R^{B}, —C(O)—C₁₋₄alkyl, C₃₋₆cycloalkyl, phenyl and 5 to 6 membered heterocyclyl;

wherein the C₃₋₆cycloalkyl, phenyl or 5 to 6 membered heterocyclyl is optionally substituted with one or more substituents independently selected from the group consisting of halogen, hydroxy, cyano, C₁₋₄alkyl, fluorinated C₁₋₄alkyl, C₁₋₄alkoxy, fluorinated C₁₋₄alkoxy, —C(O)OH, —C(O)O—(C₁₋₄alkyl), —NR^{A}R^{B}, —(C₁₋₄alkyl)-NR^{A}R^{B}, C₃₋₇cycloalkyl and 5 to 6 membered heterocyclyl;

and wherein R^{A} and R^{B} are each independently selected from the group consisting of hydrogen and C₁₋₄alkyl;

a is an integer from 1 to 3;

each R² is independently selected from the group consisting of chloro, fluoro, methyl and methoxy;

R³ is hydrogen; and R⁴ is selected from the group consisting of methyl, methoxy, and ethoxy;

alternatively, R³ and R⁴ are taken together with the carbon atom to which they are bound to form cyclopropyl;

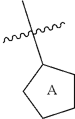

is selected from the group consisting of

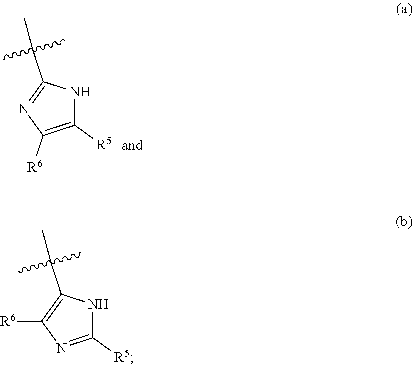

wherein R⁵ is selected from the group consisting of aryl and heterocyclyl;

wherein the aryl or heterocyclyl is optionally substituted with one or more substituents independently selected from the group consisting of halogen, hydroxy, oxo, C₁₋₄alkyl, fluorinated C₁₋₄alkyl, hydroxy substituted C₁₋₄alkyl, hydroxy substituted fluorinated C₁₋₄alkyl, C₁₋₄alkoxy, fluorinated C₁₋₄alkoxy, hydroxy substituted C₁₋₄alkoxy, hydroxy substituted fluorinated C₁₋₄alkoxy, cyano, —NR^{C}R^{D}, —(C₁₋₂alkylene)-NR^{C}R^{D}, —NR^{C}—C(O)—(C₁₋₄alkyl), —NR^{C}—C(O)—O—(C₁₋₄alkyl), —C(O)—NR^{C}—(C₁₋₄alkyl), —NR^{C}—SO₂—(C₁₋₄alkyl), cycloprop-1-yl, 1-hydroxy-cycloprop-1-yl, and —NR^{C}—C(O)-cyclopropyl; wherein R^{C} and R^{D} are each independently selected from the group consisting of hydrogen and C₁₋₄alkyl;

and R⁶ is selected from the group consisting of hydrogen, and halogen;

and tautomers, stereoisomers, isotopologues, and pharmaceutically acceptable salts thereof.

The present invention is further directed to a compound of formula (III)

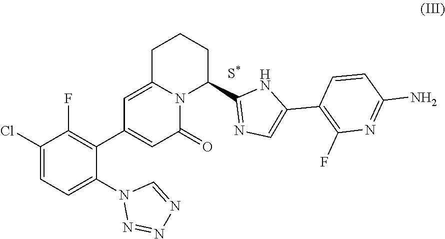 [SMILES: Nc1ccc(-c2cnc([C@@H]3CCCc4cc(-c5c(-n6cnnn6)ccc(Cl)c5F)cc(=O)n43)[nH]2)c(F)n1]

also known as (*S)-6-(5-(6-amino-2-fluoropyridin-3-yl)-1H-imidazol-2-yl)-2-(3-chloro-2-fluoro-6-(1H-tetrazol-1-yl)phenyl)-6,7,8,9-tetrahydro-4H-quinolizin-4-one; and tautomers, stereoisomers, isotopologues, and pharmaceutically acceptable salts thereof.

The present invention is further directed to compounds of formula (IV)

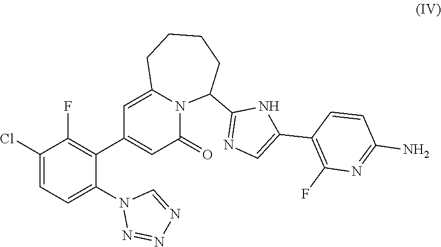 [SMILES: Nc1ccc(-c2cnc(C3CCCCc4cc(-c5c(-n6cnnn6)ccc(Cl)c5F)cc(=O)n43)[nH]2)c(F)n1]

also known as 6-(5-(6-amino-2-fluoropyridin-3-yl)-1H-imidazol-2-yl)-2-(3-chloro-2-fluoro-6-(1H-tetrazol-1-yl)phenyl)-7,8,9,10-tetrahydropyrido[1,2-a]azepin-4(6H)-one; and tautomers, stereoisomers, isotopologues, and pharmaceutically acceptable salts thereof.

The present invention is further directed to processes for the preparation of the compounds of formula (I), compounds of formula (II), compounds of formula (III) and/or compounds of formula (IV). The present invention is further directed to a compound of formula (I), compound of formula (II), compound of formula (III) and/or compound of formula (IV) prepared according to any of the process(es) described herein.

Illustrative of the invention are pharmaceutical compositions comprising a pharmaceutically acceptable carrier and a compound of formula (I), compounds of formula (II), compounds of formula (III) and/or compounds of formula (IV) as described herein. An illustration of the invention is a pharmaceutical composition made by mixing a compound of formula (I), compounds of formula (II), compounds of formula (III) and/or compounds of formula (IV) as described herein and a pharmaceutically acceptable carrier. Illustrating the invention is a process for making a pharmaceutical composition comprising mixing a compound of formula (I), compounds of formula (II), compounds of formula (III) and/or compounds of formula (IV) as described herein and a pharmaceutically acceptable carrier.

Exemplifying the invention are methods for the treatment and/or prophylaxis of thromboembolic disorders, inflammatory disorders or diseases or conditions in which plasma kallikrein activity is implicated, as described herein, comprising administering to a subject in need thereof a therapeutically effective amount of any of the compounds or pharmaceutical compositions described above.

Exemplifying the invention are methods or the treatment and/or prophylaxis of thromboembolic disorders, such as arterial cardiovascular thromboembolic disorders, venous cardiovascular thromboembolic disorders, arterial cerebrovascular thromboembolic disorders, and venous cerebrovascular thromboembolic disorders, comprising administering to a subject in need thereof a therapeutically effective amount of any of the compounds or pharmaceutical compositions described above. Examples of thromboembolic disorders include, but are not limited to, unstable angina, an acute coronary syndrome, atrial fibrillation, first myocardial infarction, recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from medical implants, devices, or procedures in which blood is exposed to an artificial surface that promotes thrombosis.

In an embodiment, the present invention is directed to a compound of formula (I), compound of formula (II), compound of formula (III) and/or compound of formula (IV) for use as a medicament. In another embodiment, the present invention is directed to a compound of formula (I), a compound of formula (II), a compound of formula (III) and/or a compound of formula (IV) for use in the treatment and/or prophylaxis of thromboembolic disorders, inflammatory disorders or diseases or conditions in which plasma kallikrein activity is implicated.

In another embodiment, the present invention is directed to a compound of formula (I), a compound of formula (II), a compound of formula (III) and/or a compound of formula (IV) for use in the treatment and/or prophylaxis of a thromboembolic disorder, such as arterial cardiovascular thromboembolic disorders, venous cardiovascular thromboembolic disorders, arterial cerebrovascular thromboembolic disorders, and venous cerebrovascular thromboembolic disorders. In another embodiment, the present invention is directed to a compound of formula (I), a compound of formula (II), a compound of formula (III) and/or a compound of formula (IV) for use in the treatment and/or prophylaxis of a thromboembolic disorder selected from the group consisting of unstable angina, an acute coronary syndrome, atrial fibrillation, first myocardial infarction, recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from medical implants, devices, or procedures in which blood is exposed to an artificial surface that promotes thrombosis. In another embodiment, the present invention is directed to a compound of formula (I) for use in the treatment and/or prophylaxis of a thromboembolic disorder selected from the group consisting of hereditary angioedema (HAE) and diabetic macular edema (DME).

In another embodiment, the present invention is directed to a composition comprising a compound of formula (I), a compound of formula (II), a compound of formula (III) and/or a compound of formula (IV) for use in the treatment and/or prophylaxis of a disorder, disease or condition as described herein. In another embodiment, the present invention is directed to a composition comprising a compound of formula (I), a compound of formula (II), a compound of formula (III) and/or a compound of formula (IV) for use in the treatment and/or prophylaxis of a thromboembolic disorder, and inflammatory disorder or a disease or condition in which plasma kallikrein activity is implicated.

In another embodiment, the present invention is directed to a composition comprising a compound of formula (I), a compound of formula (II), a compound of formula (III) and/or a compound of formula (IV) for use in the treatment and/or prophylaxis of a thromboembolic disorder, such as arterial cardiovascular thromboembolic disorders, venous cardiovascular thromboembolic disorders, arterial cerebrovascular thromboembolic disorders, and venous cerebrovascular thromboembolic disorders. In another embodiment, the present invention is directed to a composition comprising a compound of formula (I), a compound of formula (II), a compound of formula (III) and/or a compound of formula (IV) for use in the treatment and/or prophylaxis of a thromboembolic disorder selected from the group consisting of unstable angina, an acute coronary syndrome, atrial fibrillation, first myocardial infarction, recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from medical implants, devices, or procedures in which blood is exposed to an artificial surface that promotes thrombosis. In another embodiment, the present invention is directed to a composition comprising a compound of formula (I), a compound of formula (II), a compound of formula (III) and/or a compound of formula (IV) for use in the treatment and/or prophylaxis of a thromboembolic disorder such as hereditary angioedema (HAE) or diabetic macular edema (DME).

Another example of the invention is the use of any of the compounds described herein in the preparation of a medicament for the treatment and/or prophylaxis of a disorder, disease or condition as described herein. Another example of the invention is the use of any of the compounds described herein in the preparation of a medicament for the treatment and/or prophylaxis of a thromboembolic disorder, an inflammatory disorder or a disease or condition in which plasma kallikrein activity is implicated.

Another example of the invention is the use of any of the compounds described herein in the preparation of a medicament for the treatment and/or prophylaxis of a thromboembolic disorder selected from the group consisting of (a) arterial cardiovascular thromboembolic disorders, (b) venous cardiovascular thromboembolic disorders, (c) arterial cerebrovascular thromboembolic disorders, and (d) venous cerebrovascular thromboembolic disorders. Another example of the invention is the use of any of the compounds described herein in the preparation of a medicament for the treatment and/or prophylaxis of (a) unstable angina, (b) an acute coronary syndrome, (c) atrial fibrillation, (d) first myocardial infarction, (e) recurrent myocardial infarction, (f) ischemic sudden death, (g) transient ischemic attack, (h) stroke, (i) atherosclerosis, (j) peripheral occlusive arterial disease, (k) venous thrombosis, (l) deep vein thrombosis, (m) thrombophlebitis, (n) arterial embolism, (o) coronary arterial thrombosis, (p) cerebral arterial thrombosis, (q) cerebral embolism, (r) kidney embolism, (s) pulmonary embolism, or (t) thrombosis resulting from medical implants, devices, or procedures in which blood is exposed to an artificial surface that promotes thrombosis. Another example of the invention is the use of any of the compounds described herein in the preparation of a medicament for the treatment and/or prophylaxis of: (a) hereditary angioedema (HAE) or (b) diabetic macular edema (DME).

Another example of the invention is the use of any of the compounds described herein for use in a method for treating a thromboembolic, inflammatory or a disease or condition in which plasma kallikrein activity is implicated as described herein, in a subject in need thereof.

Another example of the invention is the use of any of the compounds described herein for use in a method for the treatment and/or prophylaxis of (a) arterial cardiovascular thromboembolic disorders, (b) venous cardiovascular thromboembolic disorders, (c) arterial cerebrovascular thromboembolic disorders, or (d) venous cerebrovascular thromboembolic disorders, in a subject in need thereof. Another example of the invention is the use of any of the compounds described herein for use in a method for the treatment and/or prophylaxis of (a) unstable angina, (b) an acute coronary syndrome, (c) atrial fibrillation, (d) first myocardial infarction, (e) recurrent myocardial infarction, (f) ischemic sudden death, (g) transient ischemic attack, (h) stroke, (i) atherosclerosis, (j) peripheral occlusive arterial disease, (k) venous thrombosis, (l) deep vein thrombosis, (m) thrombophlebitis, (n) arterial embolism, (o) coronary arterial thrombosis, (p) cerebral arterial thrombosis, (q) cerebral embolism, (r) kidney embolism, (s) pulmonary embolism, or (t) thrombosis resulting from medical implants, devices, or procedures in which blood is exposed to an artificial surface that promotes thrombosis, in a subject in need thereof. Another example of the invention is the use of any of the compounds described herein for use in a method for the treatment and/or prophylaxis of (a) hereditary angioedema (HAE) or (b) diabetic macular edema (DME), in a subject in need thereof.

In another example, the present invention is directed to a compound as described herein, for use in a method for the treatment and/or prophylaxis of disorders, diseases or conditions as described herein, in a subject in need thereof. In another example, the present invention is directed to a compound as described herein, for use in a method for the treatment and/or prophylaxis of a thromboembolic, inflammatory disorder, or a disease or condition in which plasma kallikrein activity is implicated, as described herein, in a subject in need thereof.

In another example, the present invention is directed to a compound as described herein, for use in methods for the treatment and/or prophylaxis of thromboembolic disorder, such as arterial cardiovascular thromboembolic disorders, venous cardiovascular thromboembolic disorders, arterial cerebrovascular thromboembolic disorders, and venous cerebrovascular thromboembolic disorders, in a subject in need thereof. In another example, the present invention is directed to a compound as described herein, for use in methods for the treatment and/or prophylaxis of unstable angina, an acute coronary syndrome, atrial fibrillation, first myocardial infarction, recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from medical implants, devices, or procedures in which blood is exposed to an artificial surface that promotes thrombosis, in a subject in need thereof. In another example, the present invention is directed to a compound as described herein, for use in a method for the treatment and/or prophylaxis of hereditary angioedema (HAE) or diabetic macular edema (DME), in a subject in need thereof.

The present invention is further directed to a compound, a composition (e.g. a pharmaceutical composition), a method of treatment, a method of preparation, a use or a method of use, as herein described.

### DETAILED DESCRIPTION OF THE INVENTION

### 1. Bicyclic Pyridinon-2-one and Pyrimidin-4-one Derivatives

Factor XIa is a plasma serine protease involved in the regulation of blood coagulation. While blood coagulation is a necessary and important part of the regulation of an organism's homeostasis, abnormal blood coagulation can also have deleterious effects. For instance, thrombosis is the formation or presence of a blood clot inside a blood vessel or cavity of the heart. Such a blood clot can lodge in a blood vessel blocking circulation and causing a heart attack or stroke. Thromboembolic disorders are the leading cause of mortality and disability in the industrialized world.

Blood clotting is a process of control of the bloodstream essential for the survival of mammals. The process of clotting, and the subsequent dissolution of the clot after wound healing has taken place, commences after vascular injury, and can be divided into four phases. The first phase, vasoconstriction or vasocontraction, can cause a decrease in blood loss in the injured area. In the next phase, platelet activation by thrombin, platelets attach to the site of the vessel wall damage and form a platelet aggregate. In the third phase, formation of clotting complexes leads to massive formation of thrombin, which converts soluble fibrinogen to fibrin by cleavage of two small peptides. In the fourth phase, after wound healing, the thrombus is dissolved by the action of the key enzyme of the endogenous fibrinolysis system, plasmin.

Two alternative pathways can lead to the formation of a fibrin clot, the intrinsic and the extrinsic pathway. These pathways are initiated by different mechanisms, but in the later phase they converge to yield a common final path of the clotting cascade. In this final path of clotting, clotting Factor X is activated. The activated Factor X is responsible for the formation of thrombin from the inactive precursor prothrombin circulating in the blood. The formation of a thrombus on the bottom of a vessel wall abnormality without a wound is the result of the intrinsic pathway. Fibrin clot formation as a response to tissue injury or an injury is the result of the extrinsic pathway. Both pathways comprise a relatively large number of proteins, which are known as clotting factors. The intrinsic pathway requires the clotting Factors V, VIII, IX, X, XI and XII and also prekallikrein, high molecular weight kininogen, calcium ions and phospholipids from platelets.

Factor XIa, a plasma serine protease involved in the regulation of blood coagulation, is initiated in vivo by the binding of tissue Factor (TF) to factor VII (FVII) to generate Factor VIIa (FVIIa). The resulting TF:FVIIa complex activates Factor IX (FIX) and Factor X (FX) that leads to the production of Factor Xa (FXa). The generated FXa catalyzes the transformation of prothrombin into small amounts of thrombin before this pathway is shut down by tissue factor pathway inhibitor (TFPI). The process of coagulation is then further propagated via the feedback activation of Factors V, VIII and XI by catalytic amounts of thrombin. (Gailani, D. et al., Arterioscler. Thromb. Vasc. Biol., 27:2507-2513 (2007)). The resulting burst of thrombin converts fibrinogen to fibrin that polymerizes to form the structural framework of a blood clot, and activates platelets, which are a key cellular component of coagulation (Hoffman, M., Blood Reviews, 17:S1-S5 (2003)). Factor XIa plays a key role in propagating this amplification loop. Epidemiological studies showed that increased circulating FXI levels in humans have been associated with increased risk for venous and arterial thrombosis, including stroke (see Quan et al. supra). In contrast, patients with congenital FXI deficiency (hemophilia C) are protected from ischemic stroke and venous thromboembolism. Therefore, Factor XIa is an attractive target for antithrombotic therapy.

In addition to stimulation via tissue factor, the coagulation system can be activated particularly on negatively charged surfaces, which include not only surface structures of foreign cells (e.g. bacteria) but also artificial surfaces such as vascular prostheses, stents and extracorporeal circulation. On the surface, initially Factor XII (FXII) is activated to Factor XIIa which subsequently activates Factor XI, attached to cell surfaces, to Factor XIa. This leads to further activation of the coagulation cascade as described above. In addition, Factor XIIa also activates bound plasma prokallikrein to plasma kallikrein (PK) which, in a potentiation loop, leads to further Factor XII activation, overall resulting in amplification of the initiation of the coagulation cascade. In addition, PK is an important bradykinin-releasing protease which leads to increased endothelial permeability. Further substrates that have been described are prorenin and prourokinase, whose activation may influence the regulatory processes of the renin-angiotensin system and fibrinolysis. The activation of PK is therefore an important link between coagulative and inflammatory processes.

COURTNEY, S., et al., in U.S. Pat. No. 9,732,085 B2, issued Aug. 15, 2017 describe pyridinone and pyrimidinone derivatives useful as inhibitors of Factor XIa.

The present invention is directed to compounds of formula (I)

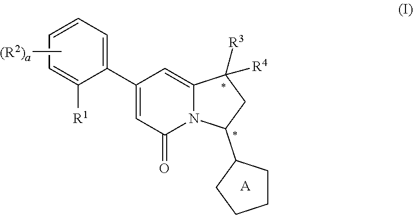

wherein a, R¹, R², R³, R⁴,

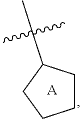

etc. are as described herein; and tautomers, stereoisomers, isotopologues, and pharmaceutically acceptable salts thereof. The compounds of formula (I) of the present invention are useful for the treatment and/or prophylaxis of thromboembolic disorders, inflammatory disorders and diseases or conditions in which plasma kallikrein activity is implicated.

The present invention is further directed to compounds of formula (II)

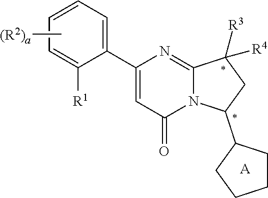

wherein a, R¹, R², R³, R⁴,

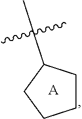

etc. are as described herein; and tautomers, stereoisomers, isotopologues, and pharmaceutically acceptable salts thereof. The compounds of formula (II) of the present invention are useful for the treatment and/or prophylaxis of thromboembolic disorders, inflammatory disorders and diseases or conditions in which plasma kallikrein activity is implicated.

The present invention is further directed to compounds of formula (III) and formula (IV), as herein described; and tautomers, stereoisomers, isotopologues, and pharmaceutically acceptable salts thereof. The compounds of (III) and formula (IV) of the present invention are useful for the treatment and/or prophylaxis of thromboembolic disorders, inflammatory disorders and diseases or conditions in which plasma kallikrein activity is implicated.

In some embodiments, the present invention is directed to compounds of formula (I) and/or compounds of formula (II) wherein

R¹ is selected from the group consisting of C₁₋₄alkyl, fluorinated C₁₋₂alkyl, C₁₋₂alkoxy, fluorinated C₁₋₂alkoxy, phenyl and 5 to 6 membered heterocyclyl; wherein the phenyl or 5 to 6 membered heterocyclyl is optionally substituted with one to two substituents independently selected from the group consisting of halogen, cyano, C₁₋₄alkyl, fluorinated C₁₋₂alkyl, C₁₋₄alkoxy, and fluorinated C₁₋₂alkoxy;

a is an integer from 1 to 3;

each R² is independently selected from the group consisting of chloro, fluoro, and methyl;

R³ is hydrogen; and R⁴ is selected from the group consisting of methyl, methoxy, and ethoxy; alternatively, R³ and R⁴ are taken together with the carbon atom to which they are bound to form cyclopropyl;

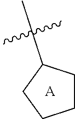

is selected from the group consisting of

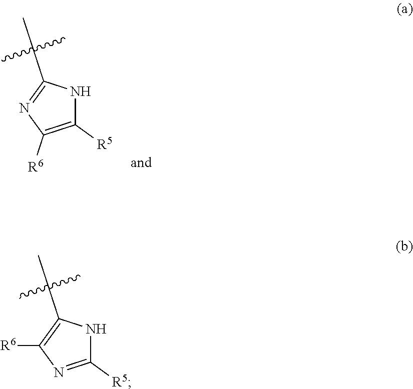

wherein R⁵ is selected from the group consisting of aryl, 5 to 6 membered heterocyclyl and 9 to 10 membered heterocyclyl; wherein the aryl, 5 to 6 membered heterocyclyl or 9 to 10 membered heterocyclyl is optionally substituted with one or more substituents independently selected from the group consisting of halogen, hydroxy, oxo, C₁₋₄alkyl, fluorinated C₁₋₄alkyl, hydroxy substituted C₁₋₄alkyl, hydroxy substituted fluorinated C₁₋₄alkyl, C₁₋₄alkoxy, fluorinated C₁₋₄alkoxy, hydroxy substituted C₁₋₄alkoxy, hydroxy substituted fluorinated C₁₋₄alkoxy, cyano, —NR^{C}R^{D}, —(C₁₋₂alkylene)-NR^{C}R^{D}, —NR^{C}—C(O)—(C₁₋₄alkyl), —NR^{C}—C(O)—O—(C₁₋₄alkyl), —C(O)—NR^{C}—(C₁₋₄alkyl), —NR^{C}—SO₂—(C₁₋₄alkyl), cycloprop-1-yl, 1-hydroxy-cycloprop-1-yl, and —NR^{C}—C(O)-cyclopropyl; wherein R^{C} and R^{D} are each independently selected from the group consisting of hydrogen and C₁₋₄alkyl;

and R⁶ is selected from the group consisting of hydrogen, and halogen;

and tautomers, stereoisomers, isotopologues, and pharmaceutically acceptable salts thereof.

In some embodiments, the present invention is directed to compounds of formula (I) and/or compounds of formula (II) wherein

R¹ is selected from the group consisting of 5 to 6 membered heterocyclyl; wherein the 5 to 6 membered heterocyclyl is optionally substituted with one to two substituents independently selected from the group consisting of halogen, and fluorinated C₁₋₂alkyl;

a is an integer from 1 to 2;

each R² is independently selected from the group consisting of chloro, and fluoro;

R³ is hydrogen; and R⁴ is selected from the group consisting of methyl, methoxy, and ethoxy; alternatively, R³ and R⁴ are taken together with the carbon atom to which they are bound to form cyclopropyl;

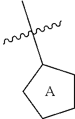

is selected from the group consisting of

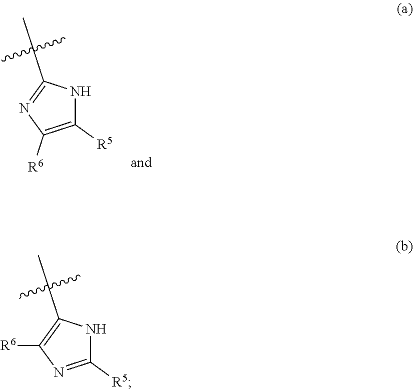

wherein R⁵ is selected from the group consisting of phenyl, 5 to 6 membered heteroaryl and 9 to 10 membered heterocyclyl; wherein the phenyl, 5 to 6 membered heteroaryl or 9 to 10 membered heterocyclyl is optionally substituted with one to three substituents independently selected from the group consisting of halogen, oxo, C₁₋₄alkyl, fluorinated C₁₋₂alkyl, hydroxy substituted C₁₋₄alkyl, C₁₋₄alkoxy, hydroxy substituted C₁₋₄alkoxy, hydroxy substituted fluorinated C₁₋₄alkoxy, cyano, —NR^{C}R^{D}, —(C₁₋₂alkylene)-NR^{C}R^{D}, —NR^{C}—C(O)—(C₁₋₄alkyl), —NR^{C}—C(O)—O—(C₁₋₂alkyl), —C(O)—NR^{C}—(C₁₋₂alkyl), —NR^{C}—SO₂—(C₁₋₄alkyl), cycloprop-1-yl, 1-hydroxy-cycloprop-1-yl, and —NR^{C}—C(O)-cyclopropyl; wherein R^{C} and R^{D} are each independently selected from the group consisting of hydrogen and C₁₋₂alkyl;

and R⁶ is selected from the group consisting of hydrogen, and halogen;

and tautomers, stereoisomers, isotopologues, and pharmaceutically acceptable salts thereof.

In some embodiments, the present invention is directed to compounds of formula (I) and/or compounds of formula (II) wherein

R¹ is selected from the group consisting of 4-(trifluoro-methyl)-1,2,3-triazol-1-yl, and 1,2,3,4-tetrazol-1-yl;

n is an integer from 1 to 2;

each R² is independently selected from the group consisting of 2-fluoro, 3-chloro, and 4-chloro;

R³ is H; and R⁴ is selected from the group consisting of methyl, R*-methyl, S*-methyl, R-methyl, S-methyl, R*-methoxy, R-methoxy, and R-ethoxy; alternatively, R³ and R⁴ are taken together with the carbon atom to which they are bound to form cyclopropyl;

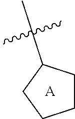

is selected from the group consisting of

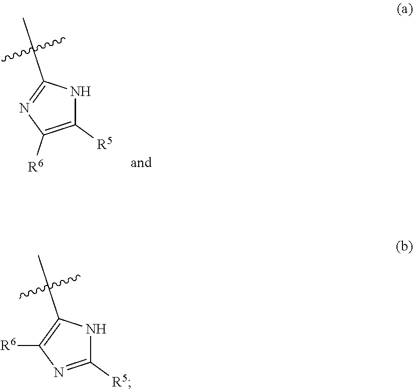

wherein R⁵ is selected from the group consisting of 4-(methoxy-carbonyl-amino)-phenyl, 4-(d3-methoxy-carbonyl-amino)-phenyl, 4-(methyl-sulfonyl-amino)-phenyl, pyrazol-4-yl, 1-methyl-pyrazol-5-yl, 1-methyl-4-cyano-pyrazol-5-yl, 1-methyl-3-amino-pyrazol-5-yl, 2-amino-thiazol-5-yl, 2-amino-4-methyl-thiazol-5-yl, 2-amino-4-(trifluoro-methyl)-thiazol-5-yl, 2-(methyl-carbonyl-amino)-thiazol-5-yl, 1,2,3-thiadiazol-5-yl, 1-methyl-1,2,3-triazol-1-yl, pyridin-3-yl-6-one, 6-amino-pyridin-3-yl, 2-fluoro-6-amino-pyridin-3-yl, 2-chloro-6-amino-pyridin-3-yl, 6-(methyl-amino-carbonyl)-pyridin-3-yl, 2-(2S*-(hydroxy)-3,3,3-trifluoro-n-propyloxy)-3-fluoro-pyridin-4-yl, 2-(2R*-(hydroxy)-3,3,3-trifluoro-n-propyloxy)-3-fluoro-pyridin-4-yl, 2-fluoro-pyridin-4-yl, 2-methyl-pyridin-4-yl, 2-cyano-pyridin-4-yl, 2-methoxy-pyridin-4-yl, 2-(trifluoro-methyl)-pyridin-4-yl, 2-(hydroxy-methyl)-3-fluoro-pyridin-4-yl, 2-(1-hydroxy-ethyl)-3-fluoro-pyridin-4-yl, 2-(methyl-amino-carbonyl)-pyridin-4-yl, 3-fluoro-6-amino-pyridin-4-yl, 2-cyano-3-methyl-pyridin-4-yl, 2-amino-3-fluoro-pyridin-4-yl, 2-(amino-methyl)-pyridin-4-yl, 2-(2-hydroxy-2-methyl-n-propyloxy)-3-fluoro-pyridin-4-yl, 2-(1-hydroxy-cycloprop-1-yl)-3-fluoro-pyridin-4-yl, 2-fluoro-3-amino-pyridin-5-yl, 2-amino-6-methyl-pyridin-5-yl, 2-amino-3-fluoro-pyridin-5-yl, 2-(methoxy-carbonyl-amino)-pyridin-5-yl, 2-(cyclopropyl-carbonyl-amino)-pyridin-5-yl, 2-amino-3-fluoro-6-methyl-pyridin-5-yl, 2-chloro-6-amino-pyrazin-3-yl, 2-fluoro-6-amino-pyrazin-3-yl, 2-amino-pyrazin-5-yl, 2-amino-6-(trifluoro-methyl)-pyrazin-5-yl, 2-amino-6-methoxy-pyrazin-5-yl, 2-amino-6-(difluoro-methyl)-pyrazin-5-yl, 2-amino-6-cyclopropyl-pyrazin-5-yl, 2-amino-6-methyl-pyrazin-5-yl, 6-amino-pyridazin-3-yl, 2-amino-pyrimidin-4-yl, 2-methoxy-pyrimidin-4-yl, 2-amino-pyrimidin-5-yl, indolin-5-yl-2-one, quinolin-7-yl-4(1H)-one, 2-methyl-indazol-5-yl, 3-amino-benzoisothiazol-6-yl, 3,4-dihydro-1,7-naphthyridin-6-yl-2(1H)-one, imidazo[1,2-a]pyridin-6-yl, and pyrazolo[1,5-a]pyridin-5-yl;

and R⁶ is selected from the group consisting of hydrogen and fluoro;

and tautomers, stereoisomers, isotopologues, and pharmaceutically acceptable salts thereof.

In some embodiments, the present invention is directed to compounds of formula (I) wherein

R¹ is 1,2,3,4-tetrazol-1-yl;

a is an integer from 1 to 2;

each R² is independently selected from the group consisting of 2-fluoro, 3-chloro and 4-chloro;

R³ is hydrogen; and R⁴ is selected from the group consisting of methyl, R*-methyl, S*-methyl, R-methyl, S-methyl, R*-methoxy, R-methoxy, and R-ethoxy; alternatively, R³ and R⁴ are taken together with the carbon atom to which they are bound to form cyclopropyl;

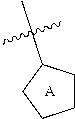

is selected from the group consisting of

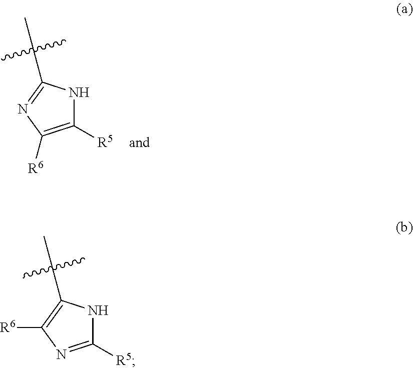

wherein R⁵ is selected from the group consisting of 4-(methoxy-carbonyl-amino)-phenyl, 4-(methyl-sulfonyl-amino)-phenyl, 1-methyl-4-cyano-pyrazol-5-yl, 1-methyl-3-amino-pyrazol-5-yl, 2-amino-thiazol-5-yl, 2-amino-4-methyl-thiazol-5-yl, 2-amino-4-(trifluoro-methyl)-thiazol-5-yl, 2-(methyl-carbonyl-amino)-thiazol-5-yl, 1-methyl-1,2,3-triazol-1-yl, pyridin-3-yl-6-one, 6-amino-pyridin-3-yl, 2-fluoro-6-amino-pyridin-3-yl, 2-chloro-6-amino-pyridin-3-yl, 6-(methyl-amino-carbonyl)-pyridin-3-yl, 2-fluoro-pyridin-4-yl, 2-methyl-pyridin-4-yl, 2-cyano-pyridin-4-yl, 2-(hydroxy-methyl)-3-fluoro-pyridin-4-yl, 2-(1-hydroxy-ethyl)-3-fluoro-pyridin-4-yl, 2-(methyl-amino-carbonyl)-pyridin-4-yl, 3-fluoro-6-amino-pyridin-4-yl, 2-cyano-3-methyl-pyridin-4-yl, 2-(amino-methyl)-pyridin-4-yl, 2-fluoro-3-amino-pyridin-5-yl, 2-amino-6-methyl-pyridin-5-yl, 2-amino-3-fluoro-pyridin-5-yl, 6-amino-pyridazin-3-yl, 2-amino-pyrimidin-4-yl, 2-methyl-indazol-5-yl, 3,4-dihydro-1,7-naphthyridin-6-yl-2(1H)-one, imidazo[1,2-a]pyridin-6-yl, and pyrazolo[1,5-a]pyridin-5-yl;

and R⁶ is selected from the group consisting of hydrogen and fluoro;

and tautomers, stereoisomers, isotopologues, and pharmaceutically acceptable salts thereof.

In some embodiments, the present invention is directed to compounds of formula (I)

R¹ is 1,2,3,4-tetrazol-1-yl;

a is an integer from 1 to 2;

each R² is independently selected from the group consisting of 2-fluoro and 3-chloro;

R³ is hydrogen; and R⁴ is selected from the group consisting of R*-methyl, S*-methyl, R-methyl, S-methyl, R-methoxy, R*-methoxy and R-ethoxy; alternatively, R³ and R⁴ are taken together with the carbon atom to which they are bound to form cyclopropyl;

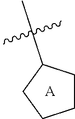

is

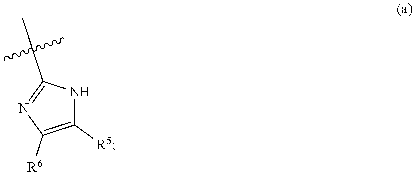

wherein R⁵ is selected from the group consisting of 4-(methoxy-carbonyl-amino)-phenyl, 4-(methyl-sulfonyl-amino)-phenyl, 1-methyl-4-cyano-pyrazol-5-yl, 1-methyl-3-amino-pyrazol-5-yl, 2-amino-thiazol-5-yl, 2-amino-4-methyl-thiazol-5-yl, 2-amino-4-(trifluoro-methyl)-thiazol-5-yl, 2-(methyl-carbonyl-amino)-thiazol-5-yl, 1-methyl-1,2,3-triazol-1-yl, pyridin-3-yl-6-one, 6-amino-pyridin-3-yl, 2-fluoro-6-amino-pyridin-3-yl, 2-chloro-6-amino-pyridin-3-yl, 6-(methyl-amino-carbonyl)-pyridin-3-yl, 2-fluoro-pyridin-4-yl, 2-methyl-pyridin-4-yl, 2-cyano-pyridin-4-yl, 2-(hydroxy-methyl)-3-fluoro-pyridin-4-yl, 2-(1-hydroxy-ethyl)-3-fluoro-pyridin-4-yl, 2-(methyl-amino-carbonyl)-pyridin-4-yl, 3-fluoro-6-amino-pyridin-4-yl, 2-cyano-3-methyl-pyridin-4-yl, 2-(amino-methyl)-pyridin-4-yl, 2-fluoro-3-amino-pyridin-5-yl, 2-amino-6-methyl-pyridin-5-yl, 2-amino-3-fluoro-pyridin-5-yl, 6-amino-pyridazin-3-yl, 2-amino-pyrimidin-4-yl, 2-methyl-indazol-5-yl, 3,4-dihydro-1,7-naphthyridin-6-yl-2(1H)-one, imidazo[1,2-a]pyridin-6-yl, and pyrazolo[1,5-a]pyridin-5-yl;

and R⁶ is selected from the group consisting of hydrogen and fluoro;

and tautomers, stereoisomers, isotopologues, and pharmaceutically acceptable salts thereof.

In some embodiments, the present invention is directed to compounds of formula (I)

R¹ is 1,2,3,4-tetrazol-1-yl;

a is 2; the two R² are 2-fluoro and 3-chloro;

R³ is H; and R⁴ is selected from the group consisting of methyl, R*-methyl, S*-methyl, and S-methyl;

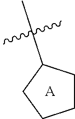

is

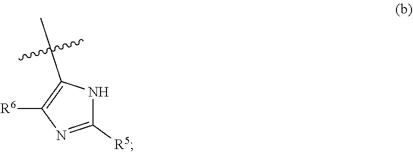

R⁵ is selected from the group consisting of 2-chloro-6-amino-pyrazin-3-yl, 2-(2S*-(hydroxy)-3,3,3-trifluoro-n-propyloxy)-3-fluoro-pyridin-4-yl, 2-(2R*-(hydroxy)-3,3,3-trifluoro-n-propyloxy)-3-fluoro-pyridin-4-yl, and 2-amino-pyrazin-5-yl;

R⁶ is selected from the group consisting of hydrogen and fluoro;

and tautomers, stereoisomers, isotopologues, and pharmaceutically acceptable salts thereof.

In some embodiments, the present invention is directed to compounds of formula (I)

R¹ is 1,2,3,4-tetrazol-1-yl;

a is an integer from 1 to 2;

each R² is independently selected from the group consisting of 2-fluoro, and 3-chloro;

R³ is hydrogen; and R⁴ is selected from the group consisting of methyl, R*-methyl, S*-methyl, R-methyl, S-methyl, R*-methoxy, R-methoxy, and R-ethoxy;

alternatively, R³ and R⁴ are taken together with the carbon atom to which they are bound to form cyclopropyl;

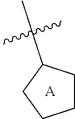

is selected from the group consisting of

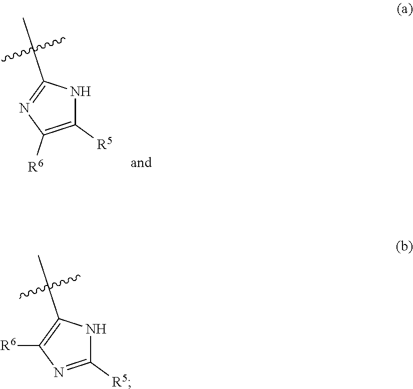

wherein R⁵ is selected from the group consisting of 4-(methoxy-carbonyl-amino)-phenyl, 4-(methyl-sulfonyl-amino)-phenyl, 2-amino-thiazol-5-yl, 2-amino-4-methyl-thiazol-5-yl, pyridin-3-yl-6-one, 2-fluoro-6-amino-pyridin-3-yl, 2-chloro-6-amino-pyridin-3-yl, 6-(methyl-amino-carbonyl)-pyridin-3-yl, 2-fluoro-pyridin-4-yl, 2-methyl-pyridin-4-yl, 2-cyano-pyridin-4-yl, 2-(hydroxy-methyl)-3-fluoro-pyridin-4-yl, 2-(1-hydroxy-ethyl)-3-fluoro-pyridin-4-yl, 2-(methyl-amino-carbonyl)-pyridin-4-yl, 3-fluoro-6-amino-pyridin-4-yl, 2-cyano-3-methyl-pyridin-4-yl, 2-(amino-methyl)-pyridin-4-yl, 2-fluoro-3-amino-pyridin-5-yl, 2-amino-6-methyl-pyridin-5-yl, 2-amino-3-fluoro-pyridin-5-yl, 6-amino-pyridazin-3-yl, 2-amino-pyrimidin-4-yl, 2-methyl-indazol-5-yl, 3,4-dihydro-1,7-naphthyridin-6-yl-2(1H)-one, imidazo[1,2-a]pyridin-6-yl, and pyrazolo[1,5-a]pyridin-5-yl;

and R⁶ is selected from the group consisting of hydrogen and fluoro;

and tautomers, stereoisomers, isotopologues, and pharmaceutically acceptable salts thereof.

In some embodiments, the present invention is directed to compounds of formula (I)

R¹ is 1,2,3,4-tetrazol-1-yl;

a is an integer from 1 to 2;

each R² is independently selected from the group consisting of 2-fluoro, and 3-chloro;

R³ is hydrogen; and R⁴ is selected from the group consisting of R*-methyl, S*-methyl, R-methyl, S-methyl, R*-methoxy, R-methoxy, and R-ethoxy;

alternatively, R³ and R⁴ are taken together with the carbon atom to which they are bound to form cyclopropyl;

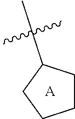

is selected from the group consisting of

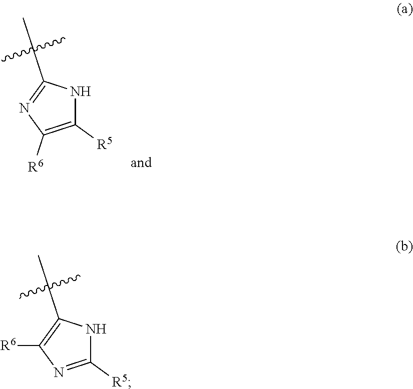

wherein R⁵ is selected from the group consisting of 4-(methoxy-carbonyl-amino)-phenyl, 4-(methyl-sulfonyl-amino)-phenyl, 2-amino-thiazol-5-yl, 2-fluoro-6-amino-pyridin-3-yl, 2-chloro-6-amino-pyridin-3-yl, 2-methyl-pyridin-4-yl, 2-(hydroxy-methyl)-3-fluoro-pyridin-4-yl, 2-cyano-3-methyl-pyridin-4-yl, 2-amino-6-methyl-pyridin-5-yl, and 3,4-dihydro-1,7-naphthyridin-6-yl-2(1H)-one;

and R⁶ is selected from the group consisting of hydrogen and fluoro;

and tautomers, stereoisomers, isotopologues, and pharmaceutically acceptable salts thereof.

In some embodiments, the present invention is directed to compounds of formula (I)

R¹ is 1,2,3,4-tetrazol-1-yl;

a is an integer from 1 to 2;

each R² is independently selected from the group consisting of 2-fluoro, and 3-chloro;

R³ is hydrogen; and R⁴ is selected from the group consisting of R*-methyl, S*-methyl, R-methyl, S-methyl, R*-methoxy, R-methoxy, and R-ethoxy; alternatively, R³ and R⁴ are taken together with the carbon atom to which they are bound to form cyclopropyl;

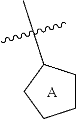

is selected from the group consisting of

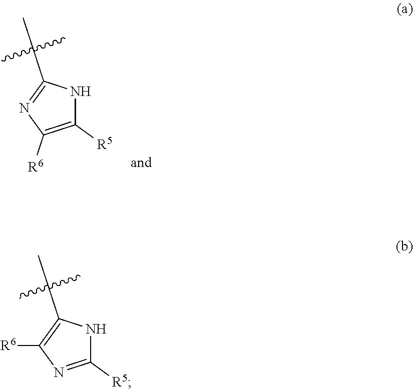

wherein R⁵ is selected from the group consisting of 4-(methoxy-carbonyl-amino)-phenyl, 2-fluoro-6-amino-pyridin-3-yl, 2-chloro-6-amino-pyridin-3-yl, and 2-amino-6-methyl-pyridin-5-yl;

and R⁶ is selected from the group consisting of hydrogen and fluoro;

and tautomers, stereoisomers, isotopologues, and pharmaceutically acceptable salts thereof.

In some embodiments, the present invention is directed to compounds of formula (II)

R¹ is selected from the group consisting of 4-(trifluoro-methyl)-1,2,3-triazol-1-yl, and 1,2,3,4-tetrazol-1-yl;

a is 2; the two R² are 2-fluoro, and 3-chloro;

R³ is H; and R⁴ is selected from the group consisting of methyl, R*-methyl, S*-methyl, R-methyl, and S-methyl; alternatively, R³ and R⁴ are taken together with the carbon atom to which they are bound to form cyclopropyl;

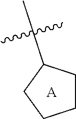

is

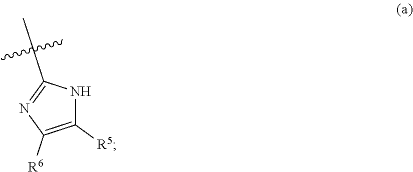

wherein R⁵ is selected from the group consisting of 4-(methoxy-carbonyl-amino)-phenyl, 4-(d3-methoxy-carbonyl-amino)-phenyl, pyrazol-4-yl, 1-methyl-pyrazol-5-yl, 2-amino-4-methyl-thiazol-5-yl, 1,2,3-thiadiazol-5-yl, 1-methyl-1,2,3-triazol-1-yl, 2-fluoro-6-amino-pyridin-3-yl, 2-chloro-6-amino-pyridin-3-yl, 2-(2S*-(hydroxy)-3,3,3-trifluoro-n-propyloxy)-3-fluoro-pyridin-4-yl, 2-(2R*-(hydroxy)-3,3,3-trifluoro-n-propyloxy)-3-fluoro-pyridin-4-yl, 2-methoxy-pyridin-4-yl, 2-(trifluoro-methyl)-pyridin-4-yl, 2-(hydroxy-methyl)-3-fluoro-pyridin-4-yl, 2-amino-3-fluoro-pyridin-4-yl, 2-(2-hydroxy-2-methyl-n-propyloxy)-3-fluoro-pyridin-4-yl, 2-(1-hydroxy-cycloprop-1-yl)-3-fluoro-pyridin-4-yl, 2-(methoxy-carbonyl-amino)-pyridin-5-yl, 2-(cyclopropyl-carbonyl-amino)-pyridin-5-yl, 2-amino-3-fluoro-6-methyl-pyridin-5-yl, 2-chloro-6-amino-pyrazin-3-yl, 2-fluoro-6-amino-pyrazin-3-yl, 2-amino-pyrazin-5-yl, 2-amino-6-(trifluoro-methyl)-pyrazin-5-yl, 2-amino-6-methoxy-pyrazin-5-yl, 2-amino-6-(difluoro-methyl)-pyrazin-5-yl, 2-amino-6-cyclopropyl-pyrazin-5-yl, 2-amino-6-methyl-pyrazin-5-yl, 2-amino-pyrimidin-4-yl, 2-methoxy-pyrimidin-4-yl, indolin-5-yl-2-one, quinolin-7-yl-4(1H)-one, 3-amino-benzoisothiazol-6-yl, and 3,4-dihydro-1,7-naphthyridin-6-yl-2(1H)-one;

and R⁶ is selected from the group consisting of hydrogen and fluoro;

and tautomers, stereoisomers, isotopologues, and pharmaceutically acceptable salts thereof.

In some embodiments, the present invention is directed to compounds of formula (II)

R¹ is selected from the group consisting of 4-(trifluoro-methyl)-1,2,3-triazol-1-yl, and 1,2,3,4-tetrazol-1-yl;

a is 2; the two R² are 2-fluoro, and 3-chloro;

R³ is H; and R⁴ is selected from the group consisting of methyl, R*-methyl, S*-methyl, R-methyl, and S-methyl; alternatively, R³ and R⁴ are taken together with the carbon atom to which they are bound to form cyclopropyl;

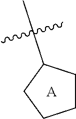

is

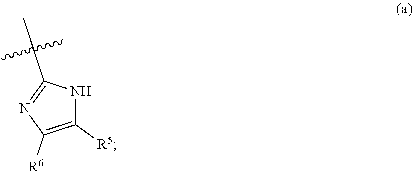

wherein R⁵ is selected from the group consisting of 4-(methoxy-carbonyl-amino)-phenyl, 4-(d3-methoxy-carbonyl-amino)-phenyl, pyrazol-4-yl, 1-methyl-pyrazol-5-yl, 2-amino-4-methyl-thiazol-5-yl, 1,2,3-thiadiazol-5-yl, 2-fluoro-6-amino-pyridin-3-yl, 2-chloro-6-amino-pyridin-3-yl, 2-(2S*-(hydroxy)-3,3,3-trifluoro-n-propyloxy)-3-fluoro-pyridin-4-yl, 2-(2R*-(hydroxy)-3,3,3-trifluoro-n-propyloxy)-3-fluoro-pyridin-4-yl, 2-methoxy-pyridin-4-yl, 2-(trifluoro-methyl)-pyridin-4-yl, 2-(hydroxy-methyl)-3-fluoro-pyridin-4-yl, 2-amino-3-fluoro-pyridin-4-yl, 2-(2-hydroxy-2-methyl-n-propyloxy)-3-fluoro-pyridin-4-yl, 2-(1-hydroxy-cycloprop-1-yl)-3-fluoro-pyridin-4-yl, 2-(methoxy-carbonyl-amino)-pyridin-5-yl, 2-(cyclopropyl-carbonyl-amino)-pyridin-5-yl, 2-chloro-6-amino-pyrazin-3-yl, 2-fluoro-6-amino-pyrazin-3-yl, 2-amino-pyrazin-5-yl, 2-amino-6-(trifluoro-methyl)-pyrazin-5-yl, 2-amino-6-methoxy-pyrazin-5-yl, 2-amino-6-(difluoro-methyl)-pyrazin-5-yl, 2-amino-6-cyclopropyl-pyrazin-5-yl, 2-amino-6-methyl-pyrazin-5-yl, 2-amino-pyrimidin-4-yl, 2-methoxy-pyrimidin-4-yl, indolin-5-yl-2-one, quinolin-7-yl-4(1H)-one, 3-amino-benzoisothiazol-6-yl, and 3,4-dihydro-1,7-naphthyridin-6-yl-2(1H)-one;

and R⁶ is selected from the group consisting of hydrogen and fluoro;

and tautomers, stereoisomers, isotopologues, and pharmaceutically acceptable salts thereof.

In some embodiments, the present invention is directed to compounds of formula (II)

R¹ is selected from the group consisting of 4-(trifluoro-methyl)-1,2,3-triazol-1-yl, and 1,2,3,4-tetrazol-1-yl;

a is 2; the two R² are 2-fluoro, and 3-chloro;

R³ is H; and R⁴ is selected from the group consisting of methyl, R*-methyl, S*-methyl, R-methyl, and S-methyl; alternatively, R³ and R⁴ are taken together with the carbon atom to which they are bound to form cyclopropyl;

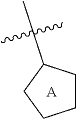

is

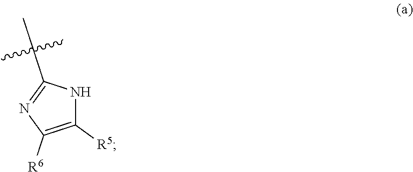

wherein R⁵ is selected from the group consisting of 4-(methoxy-carbonyl-amino)-phenyl, 4-(d3-methoxy-carbonyl-amino)-phenyl, pyrazol-4-yl, 1-methyl-pyrazol-5-yl, 2-fluoro-6-amino-pyridin-3-yl, 2-chloro-6-amino-pyridin-3-yl, 2-methoxy-pyridin-4-yl, 2-amino-3-fluoro-pyridin-4-yl, 2-(2-hydroxy-2-methyl-n-propyloxy)-3-fluoro-pyridin-4-yl, 2-(methoxy-carbonyl-amino)-pyridin-5-yl, 2-(cyclopropyl-carbonyl-amino)-pyridin-5-yl, 2-chloro-6-amino-pyrazin-3-yl, 2-fluoro-6-amino-pyrazin-3-yl, 2-amino-pyrazin-5-yl, 2-amino-6-methoxy-pyrazin-5-yl, 2-amino-6-methyl-pyrazin-5-yl, 2-amino-pyrimidin-4-yl, indolin-5-yl-2-one, 3-amino-benzoisothiazol-6-yl, and 3,4-dihydro-1,7-naphthyridin-6-yl-2(1H)-one;

and R⁶ is selected from the group consisting of hydrogen and fluoro;

and tautomers, stereoisomers, isotopologues, and pharmaceutically acceptable salts thereof.

In some embodiments, the present invention is directed to compounds of formula (II)

R¹ is selected from the group consisting of 4-(trifluoro-methyl)-1,2,3-triazol-1-yl, and 1,2,3,4-tetrazol-1-yl;

a is 2; the two R² are 2-fluoro, and 3-chloro;

R³ is H; and R⁴ is selected from the group consisting of R*-methyl, S*-methyl, R-methyl, and S-methyl; alternatively, R³ and R⁴ are taken together with the carbon atom to which they are bound to form cyclopropyl;

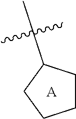

is

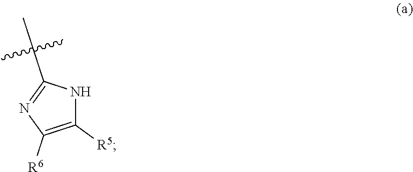

wherein R⁵ is selected from the group consisting of 4-(methoxy-carbonyl-amino)-phenyl, 4-(d3-methoxy-carbonyl-amino)-phenyl, 2-fluoro-6-amino-pyridin-3-yl, 2-chloro-6-amino-pyridin-3-yl, 2-amino-3-fluoro-pyridin-4-yl, 2-(methoxy-carbonyl-amino)-pyridin-5-yl, 2-chloro-6-amino-pyrazin-3-yl, indolin-5-yl-2-one, and 3-amino-benzoisothiazol-6-yl;

and R⁶ is selected from the group consisting of hydrogen and fluoro;

and tautomers, stereoisomers, isotopologues, and pharmaceutically acceptable salts thereof.

In some embodiments of the present invention, R¹ is selected from the group consisting of C₁₋₄alkyl, fluorinated C₁₋₂alkyl, C₁₋₂alkoxy, fluorinated C₁₋₂alkoxy, phenyl and 5 to 6 membered heterocyclyl; wherein the phenyl or 5 to 6 membered heterocyclyl is optionally substituted with one to two substituents independently selected from the group consisting of halogen, cyano, C₁₋₄alkyl, fluorinated C₁₋₂alkyl, C₁₋₄alkoxy, and fluorinated C₁₋₂alkoxy. In some embodiments of the present invention, R¹ is selected from the group consisting of 5 to 6 membered heterocyclyl; wherein the 5 to 6 membered heterocyclyl is optionally substituted with one to two substituents independently selected from the group consisting of halogen, and fluorinated C₁₋₂alkyl.

In some embodiments of the present invention, R¹ is selected from the group consisting of 4-(trifluoro-methyl)-1,2,3-triazol-1-yl, and 1,2,3,4-tetrazol-1-yl. In some embodiments of the present invention, R¹ is 1,2,3,4-tetrazol-1-yl.

In some embodiments of the present invention a is an integer from 1 to 3. In some embodiments of the present invention a is an integer from 1 to 2. In some embodiments of the present invention a is an integer from 2 to 3. In some embodiments of the present invention a 1. In some embodiments of the present invention a is 2. In some embodiments of the present invention a is 3.

In some embodiments of the present invention, each R² is independently selected from the group consisting of chloro, fluoro, and methyl. In some embodiments of the present invention, each R² is independently selected from the group consisting of chloro, and fluoro. In some embodiments of the present invention, each R² is independently selected from the group consisting of 2-fluoro, 3-chloro, and 4-chloro. In some embodiments of the present invention, each R² is independently selected from the group consisting of 2-fluoro and 3-chloro. In some embodiments of the present a is 2 and the two R² are 2-fluoro, and 3-chloro.

In some embodiments of the present invention, R³ is hydrogen; and R⁴ is selected from the group consisting of methyl, methoxy, and ethoxy. In some embodiments of the present invention, R³ is H; and R⁴ is selected from the group consisting of methyl, R*-methyl, S*-methyl, R-methyl, S-methyl, R*-methoxy, R-methoxy, and R-ethoxy. In some embodiments of the present invention R³ is hydrogen; and R⁴ is selected from the group consisting of R*-methyl, S*-methyl, R-methyl, S-methyl, R-methoxy, R*-methoxy and R-ethoxy. In some embodiments of the present invention R³ is H; and R⁴ is selected from the group consisting of methyl, R*-methyl, S*-methyl, R-methyl, and S-methyl. In some embodiments of the present invention R³ is H; and R⁴ is selected from the group consisting of methyl, R*-methyl, S*-methyl, and S-methyl. In some embodiments of the present invention R³ is H; and R⁴ is selected from the group consisting of methyl, R-methyl, and S-methyl. In some embodiments of the present invention R³ is H; and R⁴ is methyl. In some embodiments of the present invention R³ is H; and R⁴ is R-methyl. In some embodiments of the present invention R³ is H; and R⁴ is S-methyl.

In some embodiments, the present invention is directed to compounds of formula (I) wherein R³ and R⁴ are taken together with the carbon atom to which they are bound to form cyclopropyl.

In some embodiments of the present invention,

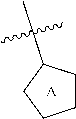

is

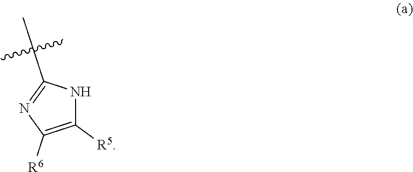

In some embodiments of the present invention

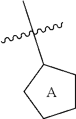

is

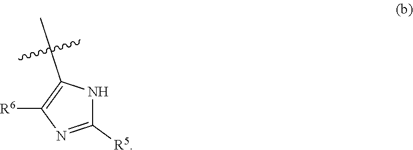

In some embodiments of the present invention R⁵ is selected from the group consisting of aryl, 5 to 6 membered heterocyclyl and 9 to 10 membered heterocyclyl; wherein the aryl, 5 to 6 membered heterocyclyl or 9 to 10 membered heterocyclyl is optionally substituted with one or more substituents independently selected from the group consisting of halogen, hydroxy, oxo, C₁₋₄alkyl, fluorinated C₁₋₄alkyl, hydroxy substituted C₁₋₄alkyl, hydroxy substituted fluorinated C₁₋₄alkyl, C₁₋₄alkoxy, fluorinated C₁₋₄alkoxy, hydroxy substituted C₁₋₄alkoxy, hydroxy substituted fluorinated C₁₋₄alkoxy, cyano, —NR^{C}R^{D}, —(C₁₋₂alkylene)-NR^{C}R^{D}, —NR^{C}—C(O)—(C₁₋₄alkyl), —NR^{C}—C(O)—O—(C₁₋₄alkyl), —C(O)—NR^{C}—(C₁₋₄alkyl), —NR^{C}—SO₂—(C₁₋₄alkyl), cycloprop-1-yl, 1-hydroxy-cycloprop-1-yl, and —NR^{C}—C(O)-cyclopropyl; wherein R^{C} and R^{D} are each independently selected from the group consisting of hydrogen and C₁₋₄alkyl.

In some embodiments of the present invention R⁵ is selected from the group consisting of phenyl, 5 to 6 membered heteroaryl and 9 to 10 membered heterocyclyl; wherein the phenyl, 5 to 6 membered heteroaryl or 9 to 10 membered heterocyclyl is optionally substituted with one to three substituents independently selected from the group consisting of halogen, oxo, C₁₋₄alkyl, fluorinated C₁₋₂alkyl, hydroxy substituted C₁₋₄alkyl, C₁₋₄alkoxy, hydroxy substituted C₁₋₄alkoxy, hydroxy substituted fluorinated C₁₋₄alkoxy, cyano, —NR^{C}R^{D}, —(C₁₋₂alkylene)-NR^{C}R^{D}, —NR^{C}—C(O)—(C₁₋₄alkyl), —NR^{C}—C(O)—O—(C₁₋₂alkyl), —C(O)—NR^{C}—(C₁₋₂alkyl), —NR^{C}—SO₂—(C₁₋₄alkyl), cycloprop-1-yl, 1-hydroxy-cycloprop-1-yl, and —NR^{C}—C(O)-cyclopropyl; wherein R^{C} and R^{D} are each independently selected from the group consisting of hydrogen and C₁₋₂alkyl.

In some embodiments of the present invention R⁵ is selected from the group consisting of 4-(methoxy-carbonyl-amino)-phenyl, 4-(d3-methoxy-carbonyl-amino)-phenyl, 4-(methyl-sulfonyl-amino)-phenyl, pyrazol-4-yl, 1-methyl-pyrazol-5-yl, 1-methyl-4-cyano-pyrazol-5-yl, 1-methyl-3-amino-pyrazol-5-yl, 2-amino-thiazol-5-yl, 2-amino-4-methyl-thiazol-5-yl, 2-amino-4-(trifluoro-methyl)-thiazol-5-yl, 2-(methyl-carbonyl-amino)-thiazol-5-yl, 1,2,3-thiadiazol-5-yl, 1-methyl-1,2,3-triazol-1-yl, pyridin-3-yl-6-one, 6-amino-pyridin-3-yl, 2-fluoro-6-amino-pyridin-3-yl, 2-chloro-6-amino-pyridin-3-yl, 6-(methyl-amino-carbonyl)-pyridin-3-yl, 2-(2S*-(hydroxy)-3,3,3-trifluoro-n-propyloxy)-3-fluoro-pyridin-4-yl, 2-(2R*-(hydroxy)-3,3,3-trifluoro-n-propyloxy)-3-fluoro-pyridin-4-yl, 2-fluoro-pyridin-4-yl, 2-methyl-pyridin-4-yl, 2-cyano-pyridin-4-yl, 2-methoxy-pyridin-4-yl, 2-(trifluoro-methyl)-pyridin-4-yl, 2-(hydroxy-methyl)-3-fluoro-pyridin-4-yl, 2-(1-hydroxy-ethyl)-3-fluoro-pyridin-4-yl, 2-(methyl-amino-carbonyl)-pyridin-4-yl, 3-fluoro-6-amino-pyridin-4-yl, 2-cyano-3-methyl-pyridin-4-yl, 2-amino-3-fluoro-pyridin-4-yl, 2-(amino-methyl)-pyridin-4-yl, 2-(2-hydroxy-2-methyl-n-propyloxy)-3-fluoro-pyridin-4-yl, 2-(1-hydroxy-cycloprop-1-yl)-3-fluoro-pyridin-4-yl, 2-fluoro-3-amino-pyridin-5-yl, 2-amino-6-methyl-pyridin-5-yl, 2-amino-3-fluoro-pyridin-5-yl, 2-(methoxy-carbonyl-amino)-pyridin-5-yl, 2-(cyclopropyl-carbonyl-amino)-pyridin-5-yl, 2-amino-3-fluoro-6-methyl-pyridin-5-yl, 2-chloro-6-amino-pyrazin-3-yl, 2-fluoro-6-amino-pyrazin-3-yl, 2-amino-pyrazin-5-yl, 2-amino-6-(trifluoro-methyl)-pyrazin-5-yl, 2-amino-6-methoxy-pyrazin-5-yl, 2-amino-6-(difluoro-methyl)-pyrazin-5-yl, 2-amino-6-cyclopropyl-pyrazin-5-yl, 2-amino-6-methyl-pyrazin-5-yl, 6-amino-pyridazin-3-yl, 2-amino-pyrimidin-4-yl, 2-methoxy-pyrimidin-4-yl, 2-amino-pyrimidin-5-yl, indolin-5-yl-2-one, quinolin-7-yl-4(1H)-one, 2-methyl-indazol-5-yl, 3-amino-benzoisothiazol-6-yl, 3,4-dihydro-1,7-naphthyridin-6-yl-2(1H)-one, imidazo[1,2-a]pyridin-6-yl, and pyrazolo[1,5-a]pyridin-5-yl.

In some embodiments of the present invention R⁵ is selected from the group consisting of 4-(methoxy-carbonyl-amino)-phenyl, 4-(methyl-sulfonyl-amino)-phenyl, 1-methyl-4-cyano-pyrazol-5-yl, 1-methyl-3-amino-pyrazol-5-yl, 2-amino-thiazol-5-yl, 2-amino-4-methyl-thiazol-5-yl, 2-amino-4-(trifluoro-methyl)-thiazol-5-yl, 2-(methyl-carbonyl-amino)-thiazol-5-yl, 1-methyl-1,2,3-triazol-1-yl, pyridin-3-yl-6-one, 6-amino-pyridin-3-yl, 2-fluoro-6-amino-pyridin-3-yl, 2-chloro-6-amino-pyridin-3-yl, 6-(methyl-amino-carbonyl)-pyridin-3-yl, 2-fluoro-pyridin-4-yl, 2-methyl-pyridin-4-yl, 2-cyano-pyridin-4-yl, 2-(hydroxy-methyl)-3-fluoro-pyridin-4-yl, 2-(1-hydroxy-ethyl)-3-fluoro-pyridin-4-yl, 2-(methyl-amino-carbonyl)-pyridin-4-yl, 3-fluoro-6-amino-pyridin-4-yl, 2-cyano-3-methyl-pyridin-4-yl, 2-(amino-methyl)-pyridin-4-yl, 2-fluoro-3-amino-pyridin-5-yl, 2-amino-6-methyl-pyridin-5-yl, 2-amino-3-fluoro-pyridin-5-yl, 6-amino-pyridazin-3-yl, 2-amino-pyrimidin-4-yl, 2-methyl-indazol-5-yl, 3,4-dihydro-1,7-naphthyridin-6-yl-2(1H)-one, imidazo[1,2-a]pyridin-6-yl, and pyrazolo[1,5-a]pyridin-5-yl.

In some embodiments of the present invention R⁵ is selected from the group consisting of 4-(methoxy-carbonyl-amino)-phenyl, 4-(methyl-sulfonyl-amino)-phenyl, 1-methyl-4-cyano-pyrazol-5-yl, 1-methyl-3-amino-pyrazol-5-yl, 2-amino-thiazol-5-yl, 2-amino-4-methyl-thiazol-5-yl, 2-amino-4-(trifluoro-methyl)-thiazol-5-yl, 2-(methyl-carbonyl-amino)-thiazol-5-yl, 1-methyl-1,2,3-triazol-1-yl, pyridin-3-yl-6-one, 6-amino-pyridin-3-yl, 2-fluoro-6-amino-pyridin-3-yl, 2-chloro-6-amino-pyridin-3-yl, 6-(methyl-amino-carbonyl)-pyridin-3-yl, 2-fluoro-pyridin-4-yl, 2-methyl-pyridin-4-yl, 2-cyano-pyridin-4-yl, 2-(hydroxy-methyl)-3-fluoro-pyridin-4-yl, 2-(1-hydroxy-ethyl)-3-fluoro-pyridin-4-yl, 2-(methyl-amino-carbonyl)-pyridin-4-yl, 3-fluoro-6-amino-pyridin-4-yl, 2-cyano-3-methyl-pyridin-4-yl, 2-(amino-methyl)-pyridin-4-yl, 2-fluoro-3-amino-pyridin-5-yl, 2-amino-6-methyl-pyridin-5-yl, 2-amino-3-fluoro-pyridin-5-yl, 6-amino-pyridazin-3-yl, 2-amino-pyrimidin-4-yl, 2-methyl-indazol-5-yl, 3,4-dihydro-1,7-naphthyridin-6-yl-2(1H)-one, imidazo[1,2-a]pyridin-6-yl, and pyrazolo[1,5-a]pyridin-5-yl.

In some embodiments of the present invention R⁵ is selected from the group consisting of 2-chloro-6-amino-pyrazin-3-yl, 2-(2S*-(hydroxy)-3,3,3-trifluoro-n-propyloxy)-3-fluoro-pyridin-4-yl, 2-(2R*-(hydroxy)-3,3,3-trifluoro-n-propyloxy)-3-fluoro-pyridin-4-yl, and 2-amino-pyrazin-5-yl. In some embodiments of the present invention R⁵ is selected from the group consisting of 4-(methoxy-carbonyl-amino)-phenyl, 4-(methyl-sulfonyl-amino)-phenyl, 2-amino-thiazol-5-yl, 2-amino-4-methyl-thiazol-5-yl, pyridin-3-yl-6-one, 2-fluoro-6-amino-pyridin-3-yl, 2-chloro-6-amino-pyridin-3-yl, 6-(methyl-amino-carbonyl)-pyridin-3-yl, 2-fluoro-pyridin-4-yl, 2-methyl-pyridin-4-yl, 2-cyano-pyridin-4-yl, 2-(hydroxy-methyl)-3-fluoro-pyridin-4-yl, 2-(1-hydroxy-ethyl)-3-fluoro-pyridin-4-yl, 2-(methyl-amino-carbonyl)-pyridin-4-yl, 3-fluoro-6-amino-pyridin-4-yl, 2-cyano-3-methyl-pyridin-4-yl, 2-(amino-methyl)-pyridin-4-yl, 2-fluoro-3-amino-pyridin-5-yl, 2-amino-6-methyl-pyridin-5-yl, 2-amino-3-fluoro-pyridin-5-yl, 6-amino-pyridazin-3-yl, 2-amino-pyrimidin-4-yl, 2-methyl-indazol-5-yl, 3,4-dihydro-1,7-naphthyridin-6-yl-2(1H)-one, imidazo[1,2-a]pyridin-6-yl, and pyrazolo[1,5-a]pyridin-5-yl.

In some embodiments of the present invention R⁵ is selected from the group consisting of 4-(methoxy-carbonyl-amino)-phenyl, 4-(methyl-sulfonyl-amino)-phenyl, 2-amino-thiazol-5-yl, 2-fluoro-6-amino-pyridin-3-yl, 2-chloro-6-amino-pyridin-3-yl, 2-methyl-pyridin-4-yl, 2-(hydroxy-methyl)-3-fluoro-pyridin-4-yl, 2-cyano-3-methyl-pyridin-4-yl, 2-amino-6-methyl-pyridin-5-yl, and 3,4-dihydro-1,7-naphthyridin-6-yl-2(1H)-one. In some embodiments of the present invention R⁵ is selected from the group consisting of 4-(methoxy-carbonyl-amino)-phenyl, 2-fluoro-6-amino-pyridin-3-yl, 2-chloro-6-amino-pyridin-3-yl, and 2-amino-6-methyl-pyridin-5-yl.

In some embodiments of the present invention R⁵ is selected from the group consisting of 4-(methoxy-carbonyl-amino)-phenyl, 4-(d3-methoxy-carbonyl-amino)-phenyl, pyrazol-4-yl, 1-methyl-pyrazol-5-yl, 2-amino-4-methyl-thiazol-5-yl, 1,2,3-thiadiazol-5-yl, 1-methyl-1,2,3-triazol-1-yl, 2-fluoro-6-amino-pyridin-3-yl, 2-chloro-6-amino-pyridin-3-yl, 2-(2S*-(hydroxy)-3,3,3-trifluoro-n-propyloxy)-3-fluoro-pyridin-4-yl, 2-(2R*-(hydroxy)-3,3,3-trifluoro-n-propyloxy)-3-fluoro-pyridin-4-yl, 2-methoxy-pyridin-4-yl, 2-(trifluoro-methyl)-pyridin-4-yl, 2-(hydroxy-methyl)-3-fluoro-pyridin-4-yl, 2-amino-3-fluoro-pyridin-4-yl, 2-(2-hydroxy-2-methyl-n-propyloxy)-3-fluoro-pyridin-4-yl, 2-(1-hydroxy-cycloprop-1-yl)-3-fluoro-pyridin-4-yl, 2-(methoxy-carbonyl-amino)-pyridin-5-yl, 2-(cyclopropyl-carbonyl-amino)-pyridin-5-yl, 2-amino-3-fluoro-6-methyl-pyridin-5-yl, 2-chloro-6-amino-pyrazin-3-yl, 2-fluoro-6-amino-pyrazin-3-yl, 2-amino-pyrazin-5-yl, 2-amino-6-(trifluoro-methyl)-pyrazin-5-yl, 2-amino-6-methoxy-pyrazin-5-yl, 2-amino-6-(difluoro-methyl)-pyrazin-5-yl, 2-amino-6-cyclopropyl-pyrazin-5-yl, 2-amino-6-methyl-pyrazin-5-yl, 2-amino-pyrimidin-4-yl, 2-methoxy-pyrimidin-4-yl, indolin-5-yl-2-one, quinolin-7-yl-4(1H)-one, 3-amino-benzoisothiazol-6-yl, and 3,4-dihydro-1,7-naphthyridin-6-yl-2(1H)-one.

In some embodiments of the present invention R⁵ is selected from the group consisting of 4-(methoxy-carbonyl-amino)-phenyl, 4-(d3-methoxy-carbonyl-amino)-phenyl, pyrazol-4-yl, 1-methyl-pyrazol-5-yl, 2-amino-4-methyl-thiazol-5-yl, 1,2,3-thiadiazol-5-yl, 2-fluoro-6-amino-pyridin-3-yl, 2-chloro-6-amino-pyridin-3-yl, 2-(2S*-(hydroxy)-3,3,3-trifluoro-n-propyloxy)-3-fluoro-pyridin-4-yl, 2-(2R*-(hydroxy)-3,3,3-trifluoro-n-propyloxy)-3-fluoro-pyridin-4-yl, 2-methoxy-pyridin-4-yl, 2-(trifluoro-methyl)-pyridin-4-yl, 2-(hydroxy-methyl)-3-fluoro-pyridin-4-yl, 2-amino-3-fluoro-pyridin-4-yl, 2-(2-hydroxy-2-methyl-n-propyloxy)-3-fluoro-pyridin-4-yl, 2-(1-hydroxy-cycloprop-1-yl)-3-fluoro-pyridin-4-yl, 2-(methoxy-carbonyl-amino)-pyridin-5-yl, 2-(cyclopropyl-carbonyl-amino)-pyridin-5-yl, 2-chloro-6-amino-pyrazin-3-yl, 2-fluoro-6-amino-pyrazin-3-yl, 2-amino-pyrazin-5-yl, 2-amino-6-(trifluoro-methyl)-pyrazin-5-yl, 2-amino-6-methoxy-pyrazin-5-yl, 2-amino-6-(difluoro-methyl)-pyrazin-5-yl, 2-amino-6-cyclopropyl-pyrazin-5-yl, 2-amino-6-methyl-pyrazin-5-yl, 2-amino-pyrimidin-4-yl, 2-methoxy-pyrimidin-4-yl, indolin-5-yl-2-one, quinolin-7-yl-4(1H)-one, 3-amino-benzoisothiazol-6-yl, and 3,4-dihydro-1,7-naphthyridin-6-yl-2(1H)-one.

In some embodiments of the present invention R⁵ is selected from the group consisting of 4-(methoxy-carbonyl-amino)-phenyl, 4-(d3-methoxy-carbonyl-amino)-phenyl, pyrazol-4-yl, 1-methyl-pyrazol-5-yl, 2-fluoro-6-amino-pyridin-3-yl, 2-chloro-6-amino-pyridin-3-yl, 2-methoxy-pyridin-4-yl, 2-amino-3-fluoro-pyridin-4-yl, 2-(2-hydroxy-2-methyl-n-propyloxy)-3-fluoro-pyridin-4-yl, 2-(methoxy-carbonyl-amino)-pyridin-5-yl, 2-(cyclopropyl-carbonyl-amino)-pyridin-5-yl, 2-chloro-6-amino-pyrazin-3-yl, 2-fluoro-6-amino-pyrazin-3-yl, 2-amino-pyrazin-5-yl, 2-amino-6-methoxy-pyrazin-5-yl, 2-amino-6-methyl-pyrazin-5-yl, 2-amino-pyrimidin-4-yl, indolin-5-yl-2-one, 3-amino-benzoisothiazol-6-yl, and 3,4-dihydro-1,7-naphthyridin-6-yl-2(1H)-one. In some embodiments of the present invention R⁵ is selected from the group consisting of 4-(methoxy-carbonyl-amino)-phenyl, 4-(d3-methoxy-carbonyl-amino)-phenyl, 2-fluoro-6-amino-pyridin-3-yl, 2-chloro-6-amino-pyridin-3-yl, 2-amino-3-fluoro-pyridin-4-yl, 2-(methoxy-carbonyl-amino)-pyridin-5-yl, 2-chloro-6-amino-pyrazin-3-yl, indolin-5-yl-2-one, and 3-amino-benzoisothiazol-6-yl.

In some embodiments of the present invention R⁶ is selected from the group consisting of hydrogen and fluoro. In some embodiments of the present invention R⁶ is hydrogen. In some embodiments of the present invention R⁶ is halogen. In some embodiments of the present invention R⁶ is fluoro.

In an embodiment, the present invention is directed to a compound of formula (I) selected from the group consisting of
Methyl(4-(2-((1S,3S)-7-(3-chloro-2-fluoro-6-(1H-tetrazol-1-yl)phenyl)-1-methyl-5-oxo-1,2,3,5-tetrahydroindolizin-3-yl)-1H-imidazol-5-yl)phenyl)carbamate;
(1S,3S)-3-(5-(6-Amino-2-chloropyridin-3-yl)-1H-imidazol-2-yl)-7-(3-chloro-2-fluoro-6-(1H-tetrazol-1-yl)phenyl)-1-methyl-2,3-dihydroindolizin-5(1H)-one;
Methyl (4-(2-((1R,3S)-7-(3-chloro-2-fluoro-6-(1H-tetrazol-1-yl)phenyl)-1-methoxy-5-oxo-1,2,3,5-tetrahydroindolizin-3-yl)-1H-imidazol-5-yl)phenyl)carbamate;
(1*R,3*R)-3-(5-(6-Amino-2-fluoropyridin-3-yl)-1H-imidazol-2-yl)-7-(3-chloro-2-fluoro-6-(1H-tetrazol-1-yl)phenyl)-1-methoxy-2,3-dihydroindolizin-5(1H)-one;
(1R,3S)-3-(5-(6-Amino-2-fluoropyridin-3-yl)-1H-imidazol-2-yl)-7-(3-chloro-2-fluoro-6-(1H-tetrazol-1-yl)phenyl)-1-ethoxy-2,3-dihydroindolizin-5(1H)-one;

and tautomers, stereoisomers, isotopologues and pharmaceutically acceptable salts thereof.

In an embodiment, the present invention is directed to a compound of formula (II) selected from the group consisting of
(6S,8S)-2-(3-Chloro-2-fluoro-6-(1H-tetrazol-1-yl)phenyl)-8-methyl-6-(5-(1-methyl-1H-pyrazol-5-yl)-1H-imidazol-2-yl)-7,8-dihydropyrrolo[1,2-a]pyrimidin-4(6H)-one;
(6*S,8*S)-6-(5-(6-Amino-2-fluoropyridin-3-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one;
Methyl (4-(2-((6*S,8*R)-2-(3-Chloro-2-fluoro-6-(1H-tetrazol-1-yl)phenyl)-8-methyl-4-oxo-4,6,7,8-tetrahydropyrrolo[1,2-a]pyrimidin-6-yl)-1H-imidazol-5-yl)phenyl)carbamate;

and tautomers, stereo-isomers, isotopologues and pharmaceutically acceptable salts thereof.

One skilled in the art will recognize that the compounds of formula (I), compounds of formula (II), compounds of formula (III) and compounds of formula (IV) may exist as tautomers, more particularly as tautomers at the imidazolyl portion of the molecule. The present invention encompasses the compounds of formula (I), compounds of formula (II), compounds of formula (III) and compounds of formula (IV) present as either tautomeric form, or as any mixture of its corresponding tautomeric forms.

One skilled in the art will recognize that, depending on substituent groups, the compounds of the present invention may contain one or more stereo-centers, including, for example, the two stereo-centers denoted by the star (“*”) symbols in the structure of the compounds of formula (I) and compounds of formula (II), as shown below

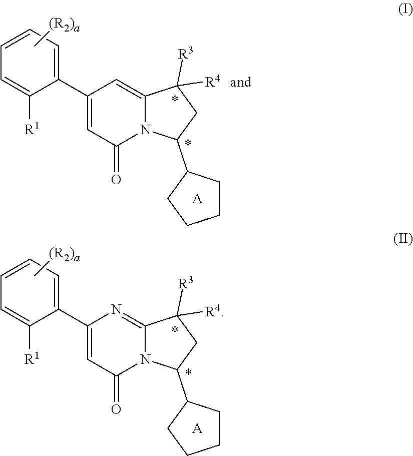

Unless otherwise noted, the starred (“*”) stereo-center at the ring carbon atom bound to R³ and R⁴ shall be referred to as the “R³/R⁴” stereo-center.

Similarly, the starred (“*”) stereo-center at the carbon atom bound to the Q

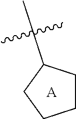

substituent group shall be referred to as the “A-ring” stereo-center.

In some embodiments, the present invention is directed to compounds of formula (I) and/or compounds of formula (II) wherein the A-Ring stereo-center is present as a racemic mixture. In some embodiments, the present invention is directed to compounds of formula (I) and/or compounds of formula (II) wherein the A-Ring stereo-center is present in an enantiomeric excess of the corresponding R-enantiomer. In some embodiments, the present invention is directed to compounds of formula (I) and/or compounds of formula (II) wherein the A-Ring stereocenter is present in an enantiomeric excess of the corresponding S-enantiomer.

In some embodiments, the present invention is directed to compounds of formula (I) and/or compounds of formula (II) wherein the A-Ring stereo-center is present in a stereo-isomeric excess of either the R- or S-enantiomer of about 55%, about 60%, about 65%, about 70%, about 75%, about 80%, about 85%, about 90%, about 91%, about 92%, about 93%, about 94%, about 95%, about 96%, about 97%, about 98% or about 99%. Preferably, the compound of formula (I) and/or compounds of formula (II) is present in a stereo-isomeric excess at the A-Ring stereo-center of greater than or equal to about 80%, preferably greater than or equal to about 90%, more preferably greater than or equal to about 93%, more preferably greater than or equal to about 95%, more preferably greater than or equal to about 97%, more preferably greater than or equal to about 98%, more preferably greater than or equal to about 99%.

In some embodiments, the present invention is directed to compounds of formula (I) and/or compounds of formula (II) wherein the R³/R⁴ stereo-center is present as a racemic mixture. In some embodiments, the present invention is directed to compounds of formula (I) and/or compounds of formula (II) wherein the R³/R⁴ stereo-center is present in an enantiomeric excess of the corresponding R-enantiomer. In some embodiments, the present invention is directed to compounds of formula (I) and/or compounds of formula (II) wherein the R³/R⁴ stereocenter is present in an enantiomeric excess of the corresponding S-enantiomer.

In some embodiments, the present invention is directed to compounds of formula (I) and/or compounds of formula (II) wherein the R³/R⁴ stereo-center is present in a stereo-isomeric excess of either the R- or S-enantiomer of about 55%, about 60%, about 65%, about 70%, about 75%, about 80%, about 85%, about 90%, about 91%, about 92%, about 93%, about 94%, about 95%, about 96%, about 97%, about 98% or about 99%. Preferably, the compound of formula (I) and/or compounds of formula (II) is present in a stereo-isomeric excess at the R³/R⁴ stereo-center of greater than or equal to about 80%, preferably greater than or equal to about 90%, more preferably greater than or equal to about 93%, more preferably greater than or equal to about 95%, more preferably greater than or equal to about 97%, more preferably greater than or equal to about 98%, more preferably greater than or equal to about 99%.

Additional embodiments of the present invention include those wherein the substituents selected for one or more of the variables defined herein (i.e. a, R¹, R², R³, R⁴,

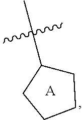

etc.) are independently selected to be any individual substituent or any subset of substituents selected from the complete list as defined herein. Additional embodiments of the present invention include those wherein the substituents selected for one or more of the variables defined herein (a, R¹, R², R³, R⁴,

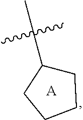

etc.) are independently selected to correspond to any of the embodiments as defined herein.

In additional embodiments, the present invention is directed to any single compound or subset of compounds independently selected from the list of representative compounds in Tables 1-3, below.

Representative compounds of the present invention are as listed in Tables 1-3, below. Unless otherwise noted, the position of R² group(s) as listed in the Tables below using the following numbering scheme:

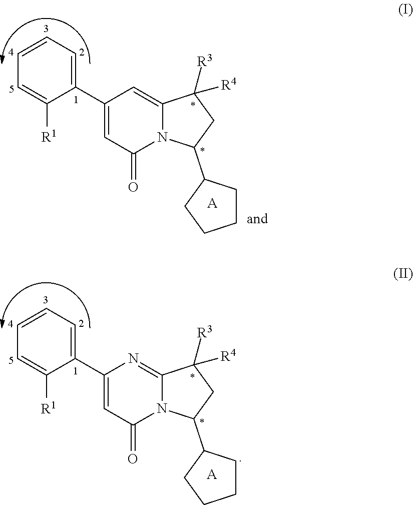

such that the R¹ substituent is bound at the 6-position and the R² substituents are bound at the 2-, 3-, 4- and/or 5-positions of the phenyl group.

In Tables 1-3, below, the column headed “A-Ring Stereo” lists the stereo-orientation at the A-Ring stereo-center; whereas the column headed “R³/R⁴ Stereo” lists the stereo-orientation at the R³/R⁴ stereo-center. Compounds prepared as racemic mixtures of the designated stereocenters are denoted as “RAC”. The S* and R* designations indicate that although the compound was prepared in an excess of one of the corresponding stereoisomers, the exact stereo-configuration was not determined. The S and R designations indicate that the compound was prepared in an excess of the corresponding S or R stereoisomer, with the exact stereo-configuration as noted.

TABLE 1
Representative Compounds of Formula (I)

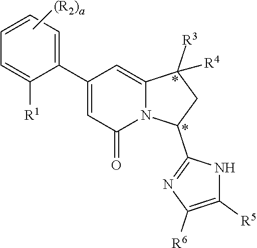

| | | | R³, R⁴ | A-Ring | | |
|---|---|---|---|---|---|---|
| ID No. | R¹ | (R²)ₐ | (w/ stereo) | Stereo | R⁵ | R⁶ |
| 1 | 1,2,3,4- | 2-fluoro, | H, R*-methyl | S* | 2-(hydroxy-methyl)- | H |
| | tetrazol-1-yl | 3-chloro | | | 3-fluoro-pyridin-4-yl | |
| 2 | 1,2,3,4- | 2-fluoro, | H, S*-methyl | S* | 2-(hydroxy-methyl)- | H |
| | tetrazol-1-yl | 3-chloro | | | 3-fluoro-pyridin-4-yl | |
| 3 | 1,2,3,4- | 3-chloro | cyclopropyl | R* | 2-fluoro-6-amino- | H |
| | tetrazol-1-yl | | | | pyridin-3-yl | |
| 4 | 1,2,3,4- | 3-chloro | cyclopropyl | S* | 2-fluoro-6-amino- | H |
| | tetrazol-1-yl | | | | pyridin-3-yl | |
| 5 | 1,2,3,4- | 2-fluoro, | cyclopropyl | S* | 2-(1-hydroxy-ethyl)- | H |
| | tetrazol-1-yl | 3-chloro | | | 3-fluoro-pyridin-4-yl | |
| 6 | 1,2,3,4- | 2-fluoro, | cyclopropyl | R* | 2-(1-hydroxy-ethyl)- | H |
| | tetrazol-1-yl | 3-chloro | | | 3-fluoro-pyridin-4-yl | |
| 7 | 1,2,3,4- | 2-fluoro, | cyclopropyl | S | 2-(hydroxy-methyl)- | H |
| | tetrazol-1-yl | 3-chloro | | | 3-fluoro-pyridin-4-yl | |
| 8 | 1,2,3,4- | 2-fluoro, | cyclopropyl | R | 2-(hydroxy-methyl)- | H |
| | tetrazol-1-yl | 3-chloro | | | 3-fluoro-pyridin-4-yl | |
| 9 | 1,2,3,4- | 2-fluoro, | H, S*-methyl | S* | 3,4-dihydro-1,7- | H |
| | tetrazol-1-yl | 3-chloro | | | naphthyridin-6-yl- | |
| | | | | | 2(1H)-one | |
| 10 | 1,2,3,4- | 2-fluoro, | H, S*-methyl | S* | 6-amino-pyridazin-3- | H |
| | tetrazol-1-yl | 3-chloro | | | yl | |
| 11 | 1,2,3,4- | 2-fluoro, | H, S-methyl | S | 4-(methyl-sulfonyl- | H |
| | tetrazol-1-yl | 3-chloro | | | amino)-phenyl | |
| 12 | 1,2,3,4- | 2-fluoro, | H, S-methyl | S | 6-(methyl-amino- | H |
| | tetrazol-1-yl | 3-chloro | | | carbonyl)-pyridin-3- | |
| | | | | | yl | |
| 13 | 1,2,3,4- | 2-fluoro, | H, S-methyl | S | 2-(methyl-amino- | H |
| | tetrazol-1-yl | 3-chloro | | | carbonyl)-pyridin-4- | |
| | | | | | yl | |
| 14 | 1,2,3,4- | 2-fluoro, | H, S-methyl | S | 1-methyl-4-cyano- | H |
| | tetrazol-1-yl | 3-chloro | | | pyrazol-5-yl | |
| 15 | 1,2,3,4- | 2-fluoro, | H, S-methyl | S | 1-methyl-3-amino- | H |
| | tetrazol-1-yl | 3-chloro | | | pyrazol-5-yl | |
| 16 | 1,2,3,4- | 2-fluoro, | H, S-methyl | S | pyridin-3-yl-6-one | H |
| | tetrazol-1-yl | 3-chloro | | | | |
| 17 | 1,2,3,4- | 2-fluoro, | cyclopropyl | R | 2-amino-thiazol-5-yl | H |
| | tetrazol-1-yl | 3-chloro | | | | |
| 18 | 1,2,3,4- | 2-fluoro, | cyclopropyl | S | 2-amino-thiazol-5-yl | H |
| | tetrazol-1-yl | 3-chloro | | | | |
| 19 | 1,2,3,4- | 2-fluoro, | H, S-methyl | S | 3-fluoro-6-amino- | H |
| | tetrazol-1-yl | 3-chloro | | | pyridin-4-yl | |
| 20 | 1,2,3,4- | 2-fluoro, | H, S-methyl | S | 2-amino-pyrimidin- | H |
| | tetrazol-1-yl | 3-chloro | | | 4-yl | |
| 21 | 1,2,3,4- | 2-fluoro, | H, S-methyl | S | 2-fluoro-3-amino- | H |
| | tetrazol-1-yl | 3-chloro | | | pyridin-5-yl | |
| 22 | 1,2,3,4- | 2-fluoro, | cyclopropyl | RAC | 2-(methyl-carbonyl- | H |
| | tetrazol-1-yl | 3-chloro | | | amino)-thiazol-5-yl | |
| 23 | 1,2,3,4- | 2-fluoro, | H, S-methyl | S | 2-amino-thiazol-5-yl | H |
| | tetrazol-1-yl | 3-chloro | | | | |
| 24 | 1,2,3,4- | 2-fluoro, | cyclopropyl | R | 2-fluoro-6-amino- | H |
| | tetrazol-1-yl | 3-chloro | | | pyridin-3-yl | |
| 25 | 1,2,3,4- | 2-fluoro, | cyclopropyl | S | 2-fluoro-6-amino- | H |
| | tetrazol-1-yl | 3-chloro | | | pyridin-3-yl | |
| 26 | 1,2,3,4- | 2-fluoro, | H, S-methyl | S | 2-amino-4-methyl- | H |
| | tetrazol-1-yl | 3-chloro | | | thiazol-5-yl | |
| 27 | 1,2,3,4- | 2-fluoro, | H, S-methyl | S | 2-amino-4-(trifluoro- | H |
| | tetrazol-1-yl | 3-chloro | | | methyl)-thiazol-5-yl | |
| 28 | 1,2,3,4- | 2-fluoro, | H, S-methyl | S | 1-methyl-1,2,3- | H |
| | tetrazol-1-yl | 3-chloro | | | triazol-1-yl | |
| 29 | 1,2,3,4- | 3-chloro | H, S-methyl | S | 2-fluoro-6-amino- | H |
| | tetrazol-1-yl | | | | pyridin-3-yl | |
| 30 | 1,2,3,4- | 2-fluoro, | H, R*-methyl | R* | 2-cyano-pyridin-4-yl | H |
| | tetrazol-1-yl | 3-chloro | | | | |
| 31 | 1,2,3,4- | 2-fluoro, | H, R*-methyl | S* | 2-cyano-3-methyl- | H |
| | tetrazol-1-yl | 3-chloro | | | pyridin-4-yl | |
| 32 | 1,2,3,4- | 2-fluoro, | H, S*-methyl | S* | 2-cyano-pyridin-4-yl | H |
| | tetrazol-1-yl | 3-chloro | | | | |
| 33 | 1,2,3,4- | 2-fluoro, | H, S*-methyl | S* | 2-cyano-3-methyl- | H |
| | tetrazol-1-yl | 3-chloro | | | pyridin-4-yl | |
| 36 | 1,2,3,4- | 4-chloro | H, S-methyl | S | 2-fluoro-6-amino- | H |
| | tetrazol-1-yl | | | | pyridin-3-yl | |
| 37 | 1,2,3,4- | 2-fluoro, | H, S*-methyl | R* | 2-chloro-6-amino- | H |
| | tetrazol-1-yl | 3-chloro | | | pyridin-3-yl | |
| 38 | 1,2,3,4- | 2-fluoro, | H, R*-methyl | R* | 2-(amino-methyl)- | H |
| | tetrazol-1-yl | 3-chloro | | | pyridin-4-yl | |
| 39 | 1,2,3,4- | 2-fluoro, | H, S*-methyl | S* | 2-(amino-methyl)- | H |
| | tetrazol-1-yl | 3-chloro | | | pyridin-4-yl | |
| 40 | 1,2,3,4- | 2-fluoro, | H, S*-methyl | S* | 6-amino-pyridin-3-yl | fluoro |
| | tetrazol-1-yl | 3-chloro | | | | |
| 41 | 1,2,3,4- | 2-fluoro, | H, R*-methyl | S* | 2-methyl-pyridin-4-yl | H |
| | tetrazol-1-yl | 3-chloro | | | | |
| 42 | 1,2,3,4- | 2-fluoro, | H, S*-methyl | R* | 2-methyl-pyridin-4-yl | H |
| | tetrazol-1-yl | 3-chloro | | | | |
| 43 | 1,2,3,4- | 2-fluoro, | H, R*-methyl | R* | 2-fluoro-pyridin-4-yl | H |
| | tetrazol-1-yl | 3-chloro | | | | |
| 44 | 1,2,3,4- | 2-fluoro, | H, S*-methyl | S* | 2-fluoro-pyridin-4-yl | H |
| | tetrazol-1-yl | 3-chloro | | | | |
| 45 | 1,2,3,4- | 2-fluoro, | H, R*-methyl | S* | 2-fluoro-pyridin-4-yl | H |
| | tetrazol-1-yl | 3-chloro | | | | |
| 46 | 1,2,3,4- | 2-fluoro, | H, S*-methyl | S* | 2-methyl-indazol-5- | H |
| | tetrazol-1-yl | 3-chloro | | | yl | |
| 47 | 1,2,3,4- | 2-fluoro, | H, R*-methyl | R* | 2-methyl-indazol-5- | H |
| | tetrazol-1-yl | 3-chloro | | | yl | |
| 48 | 1,2,3,4- | 2-fluoro, | H, S*-methyl | S* | imidazo[1,2- | H |
| | tetrazol-1-yl | 3-chloro | | | a]pyridin-6-yl | |
| 49 | 1,2,3,4- | 2-fluoro, | H, R*-methyl | R* | imidazo[1,2- | H |
| | tetrazol-1-yl | 3-chloro | | | a]pyridin-6-yl | |
| 50 | 1,2,3,4- | 2-fluoro, | H, R-methyl | S | 2-amino-6-methyl- | H |
| | tetrazol-1-yl | 3-chloro | | | pyridin-5-yl | |
| 51 | 1,2,3,4- | 2-fluoro, | H, S*-methyl | S* | 2-amino-6-methyl- | H |
| | tetrazol-1-yl | 3-chloro | | | pyridin-5-yl | |
| 52 | 1,2,3,4- | 2-fluoro, | H, R*-methyl | R* | pyrazolo[1,5- | H |
| | tetrazol-1-yl | 3-chloro | | | a]pyridin-5-yl | |
| 53 | 1,2,3,4- | 2-fluoro, | H, S*-methyl | S* | pyrazolo[1,5- | H |
| | tetrazol-1-yl | 3-chloro | | | a]pyridin-5-yl | |
| 56 | 1,2,3,4- | 2-fluoro, | H, R*-methyl | R* | 2-fluoro-6-amino- | H |
| | tetrazol-1-yl | 3-chloro | | | pyridin-3-yl | |
| 57 | 1,2,3,4- | 2-fluoro, | H, S*-methyl | S* | 2-chloro-6-amino- | H |
| | tetrazol-1-yl | 3-chloro | | | pyridin-3-yl | |
| 58 | 1,2,3,4- | 2-fluoro, | H, R-methyl | S | 2-chloro-6-amino- | H |
| | tetrazol-1-yl | 3-chloro | | | pyridin-3-yl | |
| 59 | 1,2,3,4- | 2-fluoro, | H, S-methyl | S | 2-chloro-6-amino- | H |
| | tetrazol-1-yl | 3-chloro | | | pyridin-3-yl | |
| 60 | 1,2,3,4- | 2-fluoro, | H, R*-methyl | RAC | 2-amino-3-fluoro- | H |
| | tetrazol-1-yl | 3-chloro | | | pyridin-5-yl | |
| 61 | 1,2,3,4- | 2-fluoro, | H, S*-methyl | S* | 2-amino-3-fluoro- | H |
| | tetrazol-1-yl | 3-chloro | | | pyridin-5-yl | |
| 62 | 1,2,3,4- | 2-fluoro, | H, R*-methyl | S* | 2-amino-3-fluoro- | H |
| | tetrazol-1-yl | 3-chloro | | | pyridin-5-yl | |
| 63 | 1,2,3,4- | 2-fluoro, | H, S-methyl | S | 4-(methoxy- | H |
| | tetrazol-1-yl | 3-chloro | | | carbonyl-amino)- | |
| | | | | | phenyl | |
| 64 | 1,2,3,4- | 2-fluoro, | H, S*-methyl | S* | 4-(methoxy- | H |
| | tetrazol-1-yl | 3-chloro | | | carbonyl-amino)- | |
| | | | | | phenyl | |
| 65 | 1,2,3,4- | 2-fluoro, | H, R-methyl | S | 4-(methoxy- | H |
| | tetrazol-1-yl | 3-chloro | | | carbonyl-amino)- | |
| | | | | | phenyl | |
| 66 | 1,2,3,4- | 2-fluoro, | H, R-methyl | S | 2-fluoro-6-amino- | H |
| | tetrazol-1-yl | 3-chloro | | | pyridin-3-yl | |
| 67 | 1,2,3,4- | 2-fluoro, | H, S-methyl | S | 2-fluoro-6-amino- | H |
| | tetrazol-1-yl | 3-chloro | | | pyridin-3-yl | |
| 68 | 1,2,3,4- | 2-fluoro, | H, S*-methyl | S* | 2-fluoro-6-amino- | H |
| | tetrazol-1-yl | 3-chloro | | | pyridin-3-yl | |
| 139 | 1,2,3,4- | 2-fluoro, | H, R-ethoxy | S | 2-fluoro-6-amino- | H |
| | tetrazol-1-yl | 3-chloro | | | pyridin-3-yl | |
| 151 | 1,2,3,4- | 2-fluoro, | H, R*-methoxy | R* | 2-fluoro-6-amino- | H |
| | tetrazol-1-yl | 3-chloro | | | pyridin-3-yl | |
| 152 | 1,2,3,4- | 2-fluoro, | H, R-methoxy | S | 4-(methoxy- | H |
| | tetrazol-1-yl | 3-chloro | | | carbonyl-amino)- | |
| | | | | | phenyl | |

TABLE 2
Representative Compounds of Formula (I)

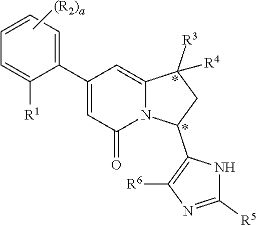

| ID | | | R³, R⁴ | A-Ring | | |
|---|---|---|---|---|---|---|
| No. | R¹ | (R²)ₐ | (w/ stereo) | Stereo | R⁵ | R⁶ |
| 34 | 1,2,3,4- | 2-fluoro, | H, S- | S | 2-fluoro-6- | fluoro |
| | tetrazol- | 3-chloro | methyl | | amino- | |
| | 1-yl | | | | pyridin-3-yl | |
| 35 | 1,2,3,4- | 2-fluoro, | H, S*- | S* | 2-fluoro-6- | fluoro |
| | tetrazol- | 3-chloro | methyl | | amino- | |
| | 1-yl | | | | pyridin-3-yl | |
| 54 | 1,2,3,4- | 2-fluoro, | H, S*- | S* | 2-fluoro-6- | H |
| | tetrazol- | 3-chloro | methyl | | amino- | |
| | 1-yl | | | | pyridin-3-yl | |
| 55 | 1,2,3,4- | 2-fluoro, | H, R*- | R* | 2-fluoro-6- | H |
| | tetrazol- | 3-chloro | methyl | | amino- | |
| | 1-yl | | | | pyridin-3-yl | |

TABLE 3
Representative Compounds of Formula (II)

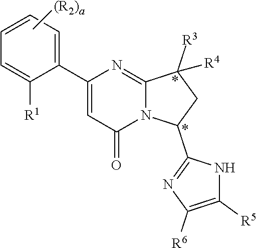

| | | | R³, R⁴ | A-Ring | | |
|---|---|---|---|---|---|---|
| ID No. | R¹ | (R²)ₐ | (w/ stereo) | Stereo | R⁵ | R⁶ |
| 69 | 1,2,3,4- | 2-fluoro, | cyclopropyl | RAC | 2-(hydroxy-methyl)- | fluoro |
| | tetrazol-1-yl | 3-chloro | | | 3-fluoro-pyridin-4-yl | |
| 70 | 1,2,3,4- | 2-fluoro, | cyclopropyl | R* | 2-(2-hydroxy-2- | H |
| | tetrazol-1-yl | 3-chloro | | | methyl-n-propyloxy)- | |
| | | | | | 3-fluoro-pyridin-4-yl | |
| 71 | 1,2,3,4- | 2-fluoro, | cyclopropyl | R* | 4-(d3-methoxy- | H |
| | tetrazol-1-yl | 3-chloro | | | carbonyl-amino)- | |
| | | | | | phenyl | |
| 72 | 1,2,3,4- | 2-fluoro, | cyclopropyl | S* | 4-(d3-methoxy- | H |
| | tetrazol-1-yl | 3-chloro | | | carbonyl-amino)- | |
| | | | | | phenyl | |
| 73 | 1,2,3,4- | 2-fluoro, | cyclopropyl | S | 2-(2S*-(hydroxy)- | H |
| | tetrazol-1-yl | 3-chloro | | | 3,3,3-trifluoro-n- | |
| | | | | | propyloxy)-3-fluoro- | |
| | | | | | pyridin-4-yl | |
| 74 | 1,2,3,4- | 2-fluoro, | cyclopropyl | S | 2-(2R*-(hydroxy)- | H |
| | tetrazol-1-yl | 3-chloro | | | 3,3,3-trifluoro-n- | |
| | | | | | propyloxy)-3-fluoro- | |
| | | | | | pyridin-4-yl | |
| 75 | 1,2,3,4- | 2-fluoro, | cyclopropyl | R* | 2-(1-hydroxy- | H |
| | tetrazol-1-yl | 3-chloro | | | cycloprop-1-yl)-3- | |
| | | | | | fluoro-pyridin-4-yl | |
| 76 | 1,2,3,4- | 2-fluoro, | cyclopropyl | S | 2-fluoro-6-amino- | fluoro |
| | tetrazol-1-yl | 3-chloro | | | pyridin-3-yl | |
| 77 | 1,2,3,4- | 2-fluoro, | H, methyl | S* | 2-(hydroxy-methyl)- | H |
| | tetrazol-1-yl | 3-chloro | | | 3-fluoro-pyridin-4-yl | |
| 78 | 1,2,3,4- | 2-fluoro, | H, methyl | R* | 2-(hydroxy-methyl)- | H |
| | tetrazol-1-yl | 3-chloro | | | 3-fluoro-pyridin-4-yl | |
| 79 | 4-(trifluoro- | 2-fluoro, | H, methyl | R | 2-amino-pyrazin-5-yl | H |
| | methyl)-1,2,3- | 3-chloro | | | | |
| | triazol-1-yl | | | | | |
| 80 | 4-(trifluoro- | 2-fluoro, | H, methyl | S | 2-amino-pyrazin-5-yl | H |
| | methyl)-1,2,3- | 3-chloro | | | | |
| | triazol-1-yl | | | | | |
| 81 | 1,2,3,4- | 2-fluoro, | cyclopropyl | S | 3-amino- | H |
| | tetrazol-1-yl | 3-chloro | | | benzoisothiazol-6-yl | |
| 82 | 1,2,3,4- | 2-fluoro, | cyclopropyl | R | 3-amino- | H |
| | tetrazol-1-yl | 3-chloro | | | benzoisothiazol-6-yl | |
| 83 | 1,2,3,4- | 2-fluoro, | H, S*-methyl | S* | 2-amino-6-(trifluoro- | H |
| | tetrazol-1-yl | 3-chloro | | | methyl)-pyrazin-5-yl | |
| 84 | 1,2,3,4- | 2-fluoro, | H, S*-methyl | S* | 2-amino-6-methoxy- | H |
| | tetrazol-1-yl | 3-chloro | | | pyrazin-5-yl | |
| 85 | 1,2,3,4- | 2-fluoro, | H, S*-methyl | R* | 2-amino-6-(difluoro- | H |
| | tetrazol-1-yl | 3-chloro | | | methyl)-pyrazin-5-yl | |
| 86 | 1,2,3,4- | 2-fluoro, | H, S*-methyl | S* | 2-amino-6-(difluoro- | H |
| | tetrazol-1-yl | 3-chloro | | | methyl)-pyrazin-5-yl | |
| 87 | 1,2,3,4- | 2-fluoro, | H, S*-methyl | R* | 2-amino-3-fluoro- | H |
| | tetrazol-1-yl | 3-chloro | | | pyridin-4-yl | |
| 88 | 1,2,3,4- | 2-fluoro, | H, S*-methyl | S* | 2-amino-3-fluoro- | H |
| | tetrazol-1-yl | 3-chloro | | | pyridin-4-yl | |
| 89 | 1,2,3,4- | 2-fluoro, | H, S-methyl | S* | 2-fluoro-6-amino- | H |
| | tetrazol-1-yl | 3-chloro | | | pyrazin-3-yl | |
| 90 | 1,2,3,4- | 2-fluoro, | H, S*-methyl | R* | 2-amino-6- | H |
| | tetrazol-1-yl | 3-chloro | | | cyclopropyl-pyrazin- | |
| | | | | | 5-yl | |
| 91 | 1,2,3,4- | 2-fluoro, | H, S*-methyl | S* | 2-amino-6- | H |
| | tetrazol-1-yl | 3-chloro | | | cyclopropyl-pyrazin- | |
| | | | | | 5-yl | |
| 92 | 1,2,3,4- | 2-fluoro, | H, S*-methyl | S* | 3,4-dihydro-1,7- | H |
| | tetrazol-1-yl | 3-chloro | | | naphthyridin-6-yl- | |
| | | | | | 2(1H)-one | |
| 93 | 1,2,3,4- | 2-fluoro, | H, S-methyl | S | 2-chloro-6-amino- | H |
| | tetrazol-1-yl | 3-chloro | | | pyrazin-3-yl | |
| 94 | 1,2,3,4- | 2-fluoro, | H, R-methyl | R* | 2-chloro-6-amino- | H |
| | tetrazol-1-yl | 3-chloro | | | pyrazin-3-yl | |
| 95 | 1,2,3,4- | 2-fluoro, | H, methyl | S | 2-amino-6-methyl- | H |
| | tetrazol-1-yl | 3-chloro | | | pyrazin-5-yl | |
| 96 | 1,2,3,4- | 2-fluoro, | H, methyl | R | 2-amino-6-methyl- | H |
| | tetrazol-1-yl | 3-chloro | | | pyrazin-5-yl | |
| 97 | 1,2,3,4- | 2-fluoro, | H, S*-methyl | S* | 2-methoxy-pyridin-4- | H |
| | tetrazol-1-yl | 3-chloro | | | yl | |
| 98 | 1,2,3,4- | 2-fluoro, | H, R*-methyl | S* | 2-methoxy-pyridin-4- | H |
| | tetrazol-1-yl | 3-chloro | | | yl | |
| 99 | 1,2,3,4- | 2-fluoro, | H, R*-methyl | R* | 2-methoxy-pyridin-4- | H |
| | tetrazol-1-yl | 3-chloro | | | yl | |
| 100 | 1,2,3,4- | 2-fluoro, | H, S*-methyl | R* | 2-methoxy-pyridin-4- | H |
| | tetrazol-1-yl | 3-chloro | | | yl | |
| 101 | 1,2,3,4- | 2-fluoro, | H, R*-methyl | S* | quinolin-7-yl-4(1H)- | H |
| | tetrazol-1-yl | 3-chloro | | | one | |
| 102 | 1,2,3,4- | 2-fluoro, | H, S*-methyl | S* | quinolin-7-yl-4(1H)- | H |
| | tetrazol-1-yl | 3-chloro | | | one | |
| 103 | 1,2,3,4- | 2-fluoro, | H, S*-methyl | S* | 2-methoxy-pyrimidin- | H |
| | tetrazol-1-yl | 3-chloro | | | 4-yl | |
| 104 | 1,2,3,4- | 2-fluoro, | H, S*-methyl | R* | 2-methoxy-pyrimidin- | H |
| | tetrazol-1-yl | 3-chloro | | | 4-yl | |
| 105 | 1,2,3,4- | 2-fluoro, | H, R*-methyl | R* | 1,2,3-thiadiazol-5-yl | H |
| | tetrazol-1-yl | 3-chloro | | | | |
| 106 | 1,2,3,4- | 2-fluoro, | H, S*-methyl | R* | 1,2,3-thiadiazol-5-yl | H |
| | tetrazol-1-yl | 3-chloro | | | | |
| 107 | 1,2,3,4- | 2-fluoro, | H, S*-methyl | S* | pyrazol-4-yl | H |
| | tetrazol-1-yl | 3-chloro | | | | |
| 108 | 1,2,3,4- | 2-fluoro, | H, R*-methyl | R* | pyrazol-4-yl | H |
| | tetrazol-1-yl | 3-chloro | | | | |
| 109 | 1,2,3,4- | 2-fluoro, | H, S*-methyl | R* | pyrazol-4-yl | H |
| | tetrazol-1-yl | 3-chloro | | | | |
| 110 | 1,2,3,4- | 2-fluoro, | H, methyl | S* | 1,2,3-thiadiazol-5-yl | H |
| | tetrazol-1-yl | 3-chloro | | | | |
| 111 | 1,2,3,4- | 2-fluoro, | H, R*-methyl | S* | pyrazol-4-yl | H |
| | tetrazol-1-yl | 3-chloro | | | | |
| 112 | 1,2,3,4- | 2-fluoro, | H, R*-methyl | R* | indolin-5-yl-2-one | H |
| | tetrazol-1-yl | 3-chloro | | | | |
| 113 | 1,2,3,4- | 2-fluoro, | H, S*-methyl | S* | indolin-5-yl-2-one | H |
| | tetrazol-1-yl | 3-chloro | | | | |
| 114 | 1,2,3,4- | 2-fluoro, | H, R*-methyl | R* | 2-amino-pyrimidin-5- | H |
| | tetrazol-1-yl | 3-chloro | | | yl | |
| 115 | 1,2,3,4- | 2-fluoro, | H, S*-methyl | R* | 2-(methoxy-carbonyl- | H |
| | tetrazol-1-yl | 3-chloro | | | amino)-pyridin-5-yl | |
| 116 | 1,2,3,4- | 2-fluoro, | H, S*-methyl | S* | 2-(methoxy-carbonyl- | H |
| | tetrazol-1-yl | 3-chloro | | | amino)-pyridin-5-yl | |
| 117 | 1,2,3,4- | 2-fluoro, | H, R*-methyl | R* | 2-(methoxy-carbonyl- | H |
| | tetrazol-1-yl | 3-chloro | | | amino)-pyridin-5-yl | |
| 118 | 1,2,3,4- | 2-fluoro, | H, R*-methyl | S* | 2-(methoxy-carbonyl- | H |
| | tetrazol-1-yl | 3-chloro | | | amino)-pyridin-5-yl | |
| 119 | 1,2,3,4- | 2-fluoro, | H, S*-methyl | S* | 2-amino-pyrimidin-5- | H |
| | tetrazol-1-yl | 3-chloro | | | yl | |
| 120 | 1,2,3,4- | 2-fluoro, | H, R*-methyl | R* | 2-(cyclopropyl- | H |
| | tetrazol-1-yl | 3-chloro | | | carbonyl-amino)- | |
| | | | | | pyridin-5-yl | |
| 121 | 1,2,3,4- | 2-fluoro, | H, S*-methyl | R* | 2-(cyclopropyl- | H |
| | tetrazol-1-yl | 3-chloro | | | carbonyl-amino)- | |
| | | | | | pyridin-5-yl | |
| 122 | 1,2,3,4- | 2-fluoro, | H, R*-methyl | S* | 2-(cyclopropyl- | H |
| | tetrazol-1-yl | 3-chloro | | | carbonyl-amino)- | |
| | | | | | pyridin-5-yl | |
| 123 | 1,2,3,4- | 2-fluoro, | H, S*-methyl | S* | 2-(cyclopropyl- | H |
| | tetrazol-1-yl | 3-chloro | | | carbonyl-amino)- | |
| | | | | | pyridin-5-yl | |
| 124 | 1,2,3,4- | 2-fluoro, | H, S*-methyl | R* | 4-(methoxy-carbonyl- | H |
| | tetrazol-1-yl | 3-chloro | | | amino)-phenyl | |
| 125 | 1,2,3,4- | 2-fluoro, | H, S*-methyl | S* | 4-(methoxy-carbonyl- | H |
| | tetrazol-1-yl | 3-chloro | | | amino)-phenyl | |
| 126 | 1,2,3,4- | 2-fluoro, | H, R*-methyl | S* | 4-(methoxy-carbonyl- | H |
| | tetrazol-1-yl | 3-chloro | | | amino)-phenyl | |
| 127 | 1,2,3,4- | 2-fluoro, | H, R*-methyl | R* | 4-(methoxy-carbonyl- | H |
| | tetrazol-1-yl | 3-chloro | | | amino)-phenyl | |
| 128 | 1,2,3,4- | 2-fluoro, | H, R*-methyl | R* | 2-amino-3-fluoro-6- | H |
| | tetrazol-1-yl | 3-chloro | | | methyl-pyridin-5-yl | |
| 129 | 1,2,3,4- | 2-fluoro, | H, S*-methyl | R* | 2-amino-3-fluoro-6- | H |
| | tetrazol-1-yl | 3-chloro | | | methyl-pyridin-5-yl | |
| 130 | 1,2,3,4- | 2-fluoro, | H, S*-methyl | S* | 2-amino-3-fluoro-6- | H |
| | tetrazol-1-yl | 3-chloro | | | methyl-pyridin-5-yl | |
| 131 | 1,2,3,4- | 2-fluoro, | cyclopropyl | S | 2-amino-4-methyl- | H |
| | tetrazol-1-yl | 3-chloro | | | thiazol-5-yl | |
| 132 | 1,2,3,4- | 2-fluoro, | cyclopropyl | R | 2-amino-4-methyl- | H |
| | tetrazol-1-yl | 3-chloro | | | thiazol-5-yl | |
| 133 | 1,2,3,4- | 2-fluoro, | cyclopropyl | R | 2-fluoro-6-amino- | H |
| | tetrazol-1-yl | 3-chloro | | | pyridin-3-yl | |
| 134 | 1,2,3,4- | 2-fluoro, | cyclopropyl | S | 2-fluoro-6-amino- | H |
| | tetrazol-1-yl | 3-chloro | | | pyridin-3-yl | |
| 135 | 1,2,3,4- | 2-fluoro, | H, R*-methyl | R* | 1-methyl-1,2,3- | H |
| | tetrazol-1-yl | 3-chloro | | | triazol-1-yl | |
| 136 | 1,2,3,4- | 2-fluoro, | H, S*-methyl | S* | 1-methyl-1,2,3- | H |
| | tetrazol-1-yl | 3-chloro | | | triazol-1-yl | |
| 137 | 1,2,3,4- | 2-fluoro, | H, R*-methyl | S* | 2-fluoro-6-amino- | H |
| | tetrazol-1-yl | 3-chloro | | | pyridin-3-yl | |
| 138 | 1,2,3,4- | 2-fluoro, | H, R*-methyl | R* | 2-fluoro-6-amino- | H |
| | tetrazol-1-yl | 3-chloro | | | pyridin-3-yl | |
| 140 | 1,2,3,4- | 2-fluoro, | H, R*-methyl | R* | 2-chloro-6-amino- | H |
| | tetrazol-1-yl | 3-chloro | | | pyridin-3-yl | |
| 141 | 1,2,3,4- | 2-fluoro, | H, R-methyl | S | 2-chloro-6-amino- | H |
| | tetrazol-1-yl | 3-chloro | | | pyridin-3-yl | |
| 142 | 1,2,3,4- | 2-fluoro, | H, S-methyl | S | 2-chloro-6-amino- | H |
| | tetrazol-1-yl | 3-chloro | | | pyridin-3-yl | |
| 143 | 1,2,3,4- | 2-fluoro, | H, R*-methyl | R* | 1-methyl-pyrazol-5-yl | H |
| | tetrazol-1-yl | 3-chloro | | | | |
| 144 | 1,2,3,4- | 2-fluoro, | H, S*-methyl | S* | 1-methyl-pyrazol-5-yl | H |
| | tetrazol-1-yl | 3-chloro | | | | |
| 145 | 1,2,3,4- | 2-fluoro, | H, R*-methyl | S* | 1-methyl-pyrazol-5-yl | H |
| | tetrazol-1-yl | 3-chloro | | | | |
| 146 | 1,2,3,4- | 2-fluoro, | H, S-methyl | S | 1-methyl-pyrazol-5-yl | H |
| | tetrazol-1-yl | 3-chloro | | | | |
| 147 | 1,2,3,4- | 2-fluoro, | H, R*-methyl | S* | 2-(trifluoro-methyl)- | H |
| | tetrazol-1-yl | 3-chloro | | | pyridin-4-yl | |
| 148 | 1,2,3,4- | 2-fluoro, | H, S*-methyl | S* | 2-(trifluoro-methyl)- | H |
| | tetrazol-1-yl | 3-chloro | | | pyridin-4-yl | |
| 149 | 1,2,3,4- | 2-fluoro, | H, S-methyl | S | 2-fluoro-6-amino- | H |
| | tetrazol-1-yl | 3-chloro | | | pyridin-3-yl | |
| 150 | 1,2,3,4- | 2-fluoro, | H, S*-methyl | S* | 2-fluoro-6-amino- | H |
| | tetrazol-1-yl | 3-chloro | | | pyridin-3-yl | |

### 2. General Synthesis Schemes

Compounds of formula (I) and compounds of formula (II) of the present invention may be prepared as described in the general synthesis schemes and Examples which follow hereinafter, selecting and substituting suitable reagents and conditions, as would be well within the skill of persons versed in the art. Additionally, the preparation of any starting materials used in the schemes and synthesis examples which follow hereinafter is well within the skill of persons versed in the art.

Intermediates in the synthesis of the compounds of formula (I) and compounds of formula (II) of the present invention include compounds of formula (M1)

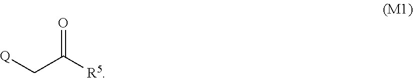

wherein Q is bromo or chloro. Some compounds of formula (M1) are known or may be prepared according to known methods, as would be recognized by those skilled in the art. Compounds of formula (M1) may further be prepared as described in the Schemes and Examples which follow herein.

Intermediate compounds of formula (M1) wherein Q is bromo may be prepared as described in Scheme 1A and Scheme 1B, below.

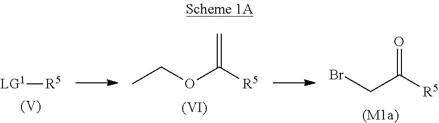

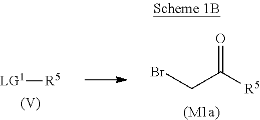

According to Scheme 1A, a suitably substituted compound of formula (V), wherein LG¹ is a suitably selected leaving group for example Br, a known compound or compound prepared by known methods is reacted with dibutyl(1-ethoxyvinyl)(pentyl)stannane, a known compound; in the presence of a suitably selected coupling agent such as Pd(dppf)Cl₂, Pd(PPh₃)Cl₂, Pd(PPh₃)₄, and the like; in a suitably selected solvent such as 1,4-dioxane, THF, toluene, and the like; at an elevated temperature, for example at about 90-100° C.; to yield the corresponding compound of formula (VI).

The compound of formula (VI) is reacted with a suitably selected source of bromine such as NBS, HBr, Br₂, pyridine.HBr, and the like; in a suitably selected solvent or mixture of solvents such as a mixture of THF/water, DCM, acetonitrile, Et₂O, and the like; at about room temperature; to yield the corresponding compound of formula (M1). One skilled in the art will recognize that wherein the source of bromine is for example a mixture of HBr and Br, pyridine.HBr, and the like; then the reaction of the compound of formula (VI) with the source of bromine is completed in the presence of a suitably selected acid such as AcOH, formic acid, TFA, and the like.

One skilled in the art will recognize that compounds of formula (M1) wherein Q is chloro may be similarly prepared as described in Scheme 1A above, by reacting the compound of formula (VI) with a suitably selected chlorinating agent such as NCS, and the like; in a suitably selected solvent such as DCM, DCE, THF, Et₂O, and the like; to yield the corresponding compound of formula (M1) wherein Q is chloro.

Alternatively, according to Scheme 1B, a compound of formula (V), wherein LG⁴ is —C(O)Cl, a known compound or compound prepared by known methods, is reacted with a suitably selected agent such as trimethylsilyldiazomethane (TMSCHN₂) and the like; in a suitably selected solvent such as acetonitrile and the like; and then reacted with a suitably selected source of bromine such a HBr/water and the like; at about room temperature; to yield the corresponding compound of formula (M1a).

Intermediates in the synthesis of the compounds of formula (I) and compounds of formula (II) further include compounds of formula (M2)

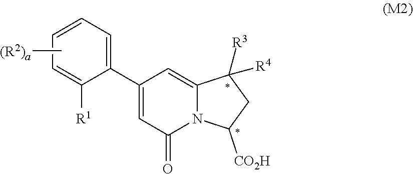

and compounds of formula (M3)

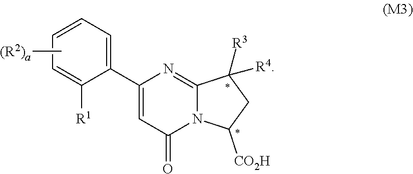

respectively. Some compounds of formula (M2) and formula (M3) are known or may be prepared according to known methods, as would be recognized by those skilled in the art. Compounds of formula (M2) and formula (M3) may further be prepared as described in the Schemes and Examples which follow herein.

Intermediate compounds of formula (M2) wherein R³ and R⁴ are taken together with the carbon atom to which they are attached to form cyclopropyl may be prepared as described in Scheme 2, below.

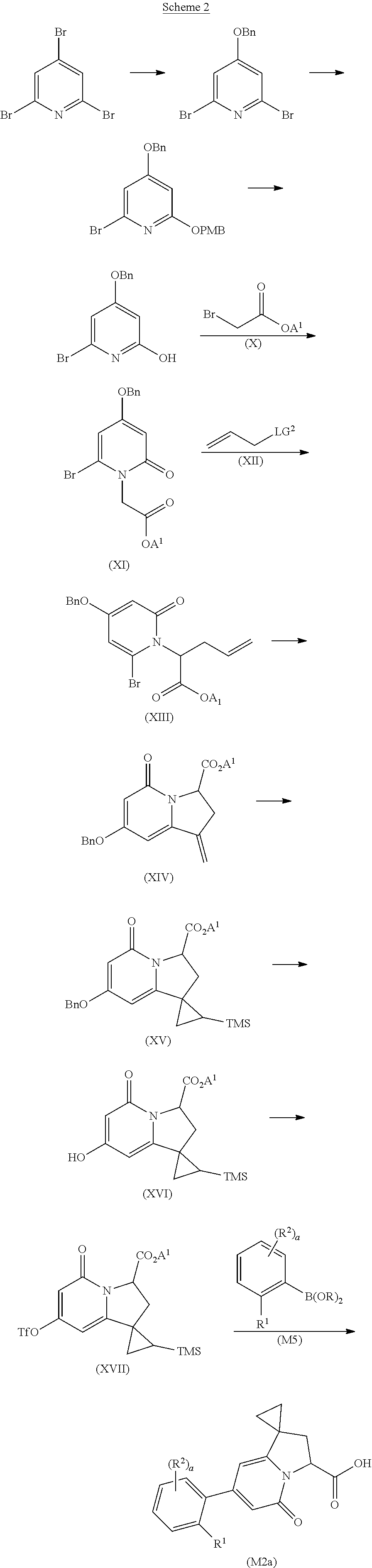

Accordingly, 2,4,6-tribromopyridine, a known compound, is reacted with one equivalent of benzyl alcohol in the presence of a suitably selected base such as NaH, KOt-Bu, and the like; in a suitably selected solvent or mixture of solvents such as a mixture of 1,4-dioxane/water, THF, and the like; at a reduced temperature, for example at about −20° C.; to yield 4-(benzyloxy)-2,6-dibromopyridine.

The 4-(benzyloxy)-2,6-dibromopyridine is reacted with a second reagent such as (methoxyphenyl)methanol, a known compound, in the presence of a suitably selected base such as NaH, KOt-Bu, NaOt-Bu, and the like; in a suitably selected solvent or mixture of solvents such as a mixture of 1,4-dioxane/water, THF, Et₂O, at a reduced temperature, for example at about −20° C.; to yield 4-(benzyloxy)-2-bromo-6-((4-methoxybenzyl)oxy)pyridine.

The 4-(benzyloxy)-2-bromo-6-((4-methoxybenzyl)oxy)pyridine is selectively de-protected by reacting with a suitably selected acid such as TFA, HCl, and the like; in a suitably selected solvent such as DCM, 1,4-dioxane, and the like; to yield 4-(benzyloxy)-6-bromopyridin-2-ol.

The 4-(benzyloxy)-6-bromopyridin-2-ol is reacted with a suitably selected alkyl bromoacetate, a compound of formula (X), wherein A¹ is C₁₋₄alkyl, preferably methyl or ethyl, such as ethyl bromoacetate, and the like, a known compound; in the presence of a suitably selected base such as NaH, Cs₂CO₃, and the like; in the presence of a suitably selected lithium halide such as LiBr, LiI, and the like; in a suitably selected solvent or mixture of solvents such as a mixture of DME/DMF, DMSO, acetonitrile, and the like; at an elevated temperature, for example at about 65° C.; to yield the corresponding compound of formula (XI), wherein A¹ is the corresponding alkyl.

The compound of formula (XI) is reacted with a suitably substituted compound of formula (XII), wherein LG² is a suitably selected leaving group such as I, Br, and the like; in the presence of a suitably selected base such as LiHMDS, KHMDS, and the like; in a suitably selected solvent such as THF, Et₂O, and the like; at a reduced temperature, for example at about −70° C.; to yield the corresponding compound of formula (XIII).

The compound of formula (XIII) is reacted in the presence of a suitably selected catalyst such as Pd(OAc)₂, Pd₂dba₃, and the like; in the presence of a ligand such as PPh₃, Dave-Phos ligand, and the like; in the presence of a suitably selected organic amine base such as TEA, DIPEA, pyridine, and the like; in a suitably selected solvent such as acetonitrile, DMF, and the like; at an elevated temperature, for example at about 80° C.; to yield the corresponding compound of formula (XIV).

The compound of formula (XIV) is reacted with a suitably selected reagent such as TMSCHN₂, and the like; in the presence of a suitably selected catalyst such as CuPC, Rh₂(OAc)₂, CuCl, and the like; in a suitably selected solvent such as DCE, DCM, and the like; at an elevated temperature, for example at about 80° C.; to yield the corresponding compound of formula (XV).

The compound of formula (XV) is reacted to selectively de-protect, according to known methods; for example, by reacting with H₂(g) in the presence of a suitable catalysts such as Pd/C, in a suitable solvent such as MeOH, at about room temperature; to yield the corresponding compound of formula (XVI).

The compound of formula (XVI) is reacted to derivatize the hydroxy group, according to known methods; for example by reacting with 1,1,1-trifluoro-N-phenyl-N-((trifluoromethyl)sulfonyl)methanesulfonamide, a known compound, in the presence of TEA, in DCM at about room temperature; to yield the corresponding compound of formula (XVII), wherein PG¹ is the corresponding protecting group (e.g. trifluoromethylsulfonyl if using the above exemplified conditions).

The compound of formula (XVII) is reacted with a suitably substituted compound of formula (M5), wherein B(OR)₂ is —B(OC₁₋₄alkyl)₂ or

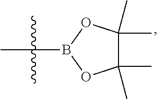 [SMILES: *B1OC(C)(C)C(C)(C)O1]

a known compound, compound prepared by known methods or compound prepared as described herein; in the presence of a suitably selected agent such as Pd(PPh₃)₄, PdCl₂dppf, Pd₂dba₃, and the like; in the presence of a suitably selected agent such as CsF, K₂CO₃, and the like; in a suitably selected solvent such as 1,4-dioxane, toluene, and the like; at an elevated temperature, for example at about reflux temperature; to yield the corresponding compound of formula (XVIII).

The compound of formula (XVIII) is reacted (to remove TMS and simultaneously convert the alkyl ester to the corresponding carboxylic acid) with a suitably selected agent such as TBAF, pyridine.HF, and the like; neat or in a suitably selected solvent; at an elevated temperature, for example at about 70° C.; to yield the corresponding compound of formula (M2a), the compound of formula (M2) wherein R³ and R⁴ are taken together with the carbon atom to which they are bound to form cyclopropyl.

Intermediate compounds of formula (M3) wherein R³ and R⁴ are taken together with the carbon atom to which they are attached to form cyclopropyl may be prepared as described in Scheme 3, below.

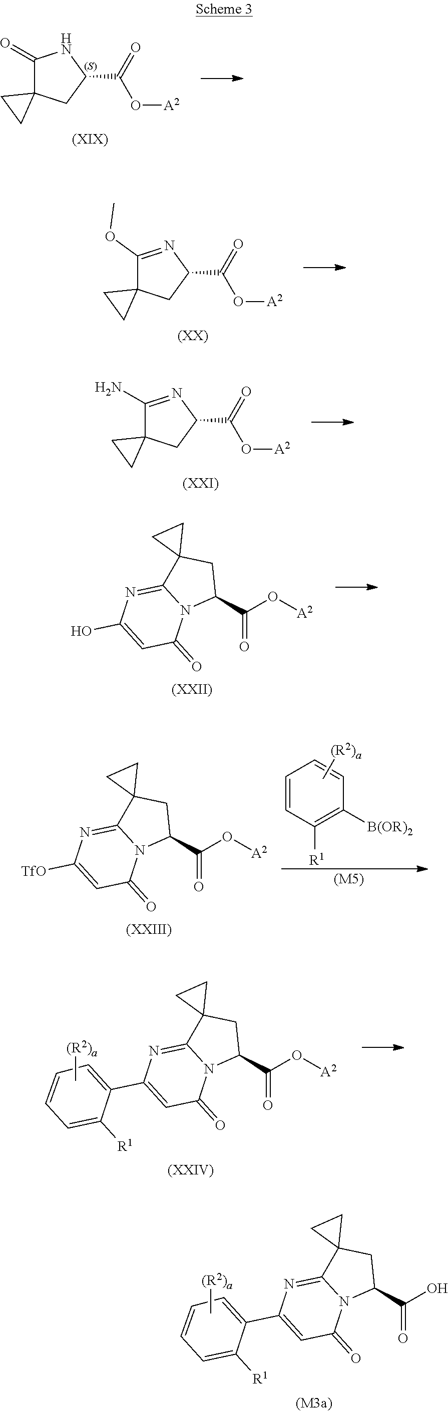

Accordingly, a suitably substituted compound of formula (XIX) wherein A² is C₁₋₄alkyl, preferably methyl or ethyl, a known compound or compound prepared by known methods, is reacted with trimethyloxonium tetrafluoroborate, a known compound or compound prepared by known methods; in a suitably selected solvent such as DCM, DCE, and the like; at about room temperature; to yield the corresponding compound of formula (XX).

The compound of formula (XX) is reacted with a suitably selected source of nitrogen such as NH₄Cl, NH₃.H₂O, and the like; in a suitably selected solvent such as ethanol, 1,4-dioxane, and the like; at an elevated temperature, for example at about reflux temperature; to yield the corresponding compound of formula (XXI).

The compound of formula (XXI) is reacted with potassium 3-ethoxy-3-oxopropanoate, a known compound; in the present of a suitably selected agent such as EDCl, HATU, and the like; in the presence of a suitably selected organic amine base such as TEA, DIPEA, pyridine, and the like; in a suitably selected solvent such as DMF, 1,4-dioxane, and the like; at an elevated temperature, for example at about 60° C.; to yield the corresponding compound of formula (XXII).

The compound of formula (XXII) is reacted to derivatize the hydroxy group, according to known methods; for example by reacting with 1,1,1-trifluoro-N-phenyl-N-((trifluoromethyl)sulfonyl)methanesulfonamide, a known compound, in the presence of TEA, in DCM at about room temperature; to yield the corresponding compound of formula (XXIII), wherein PG¹ is the corresponding protecting group (e.g. trifluoromethylsulfonyl if using the above exemplified conditions).

The compound of formula (XXIII) is reacted with a suitably substituted compound of formula (M5), wherein B(OR)₂ is —B(OC₁₋₄alkyl)₂ or,

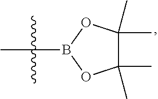 [SMILES: *B1OC(C)(C)C(C)(C)O1]

a known compound, compound prepared by known methods or compound prepared as described herein; in the presence of a suitably selected agent such as Pd(PPh₃)₄, PdCl₂dppf, Pd₂dba₃, and the like; in the presence of a suitably selected agent such as CsF, K₂CO₃, and the like; in a suitably selected solvent such as 1,4-dioxane, toluene, and the like; at an elevated temperature, for example at about reflux temperature; to yield the corresponding compound of formula (XXIV).

The compound of formula (XXIV) is reacted (to remove TMS and simultaneously convert the alkyl ester to the corresponding carboxylic acid) with a suitably selected base such as LiOH, NaOH, and the like; in a suitably selected solvent or mixture of solvents such as a mixture of methanol/water, THE/water, and the like; at about room temperature; to yield the corresponding compound of formula (M3a), the compound of formula (M3) wherein R³ and R⁴ are taken together with the carbon atom to which they are bound to form cyclopropyl.

One skilled in the art will recognize that compounds of formula (M3a) wherein the stereo-center is present in the S- or R-configuration may be similarly prepared as described in Scheme 3 above, by reacting starting from the corresponding enantiomerically enriched compound of formula (XIX).

Intermediate compounds of formula (M3) wherein R³ is hydrogen and R⁴ is hydrogen, methyl, methoxy, or ethoxy may be prepared as described in Scheme 4, below.

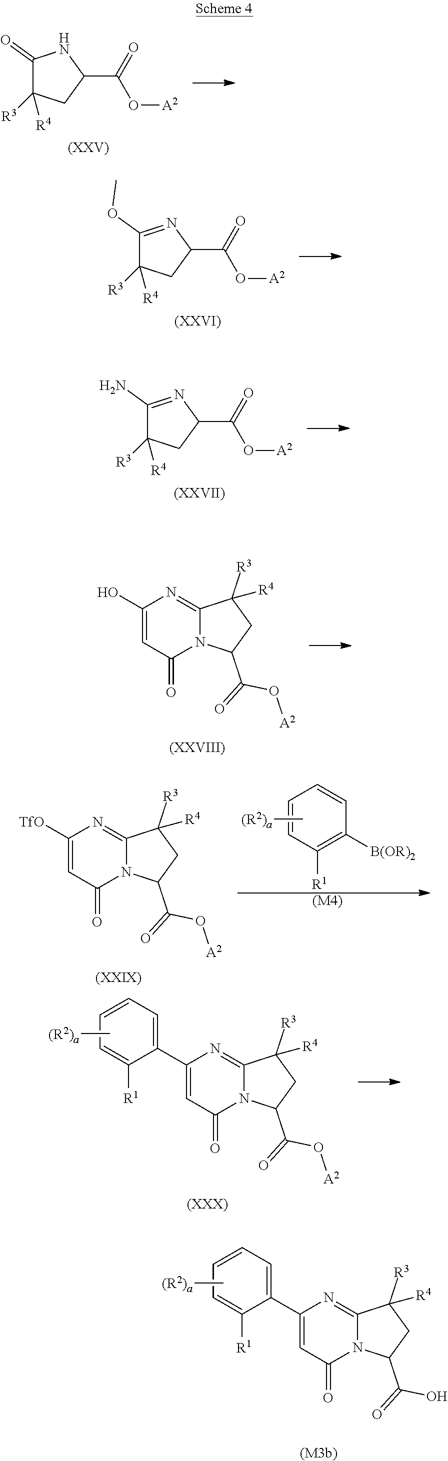

Accordingly, a suitably substituted compound of formula (XXV), wherein A² is C₁₋₄alkyl, preferably methyl or ethyl, a known compound or compound prepared by known methods, is reacted with a suitably selected agent such as trimethyloxonium tetrafluoroborate, and the like; in a suitably selected solvent such as DCM, DCE, and the like; at about room temperature; to yield the corresponding compound of formula (XXVI).

The compound of formula (XXVI) is reacted with a suitably selected source of ammonia such as NH₄Cl, NH₃.H₂O, and the like; in a suitably selected solvent such as ethanol, 1,4-dioxane, and the like; at an elevated temperature, for example at about 80° C.; to yield the corresponding compound of formula (XXVII).

The compound of formula (XXVII) is reacted with ethyl 3-chloro-3-oxopropanoate, a known compound, in the presence of a suitably selected organic amine base such as TEA, DIEA, pyridine, and the like; in a suitably selected solvent such as 1,4-dioxane, DCM, THF, and the like; at an elevated temperature, for example at about 80° C.; to yield the corresponding compound of formula (XXVIII).

The compound of formula (XXVIII) is reacted to derivatize the hydroxy group, according to known methods; for example by reacting with 1,1,1-trifluoro-N-phenyl-N-((trifluoromethyl)sulfonyl)methanesulfonamide, a known compound, in the presence of TEA, in DCM at about room temperature; to yield the corresponding compound of formula (XXIX), wherein PG² is the corresponding protecting group (e.g. trifluoromethylsulfonyl if using the above exemplified conditions).

The compound of formula (XXIX) is reacted with a suitably substituted compound of formula (M4), a known compound, compound prepared by known methods, or compound prepared as described herein, and wherein —B(OR)₂ is for example, —B(OH)₂, —B(OCH₃)₂,

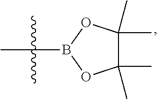 [SMILES: *B1OC(C)(C)C(C)(C)O1]

and the like, a known compound or compound prepared by known methods; in the presence of a suitably selected coupling agent such as Pd(dppf)Cl₂, Pd(PPh₃)₄, Pd(OAc)₂, and the like; in the presence of a suitably selected base such as K₂CO₃, Cs₂CO₃, K₃PO₄, and the like; in a suitably selected solvent or mixture of solvents such as a mixture of 1,4-dioxane and water, DMF, toluene/water, and the like; at an elevated temperature, such as about 80° C.; to yield the corresponding compound of formula (XXX).

The compound of formula (XXX) is reacted with a suitably selected base such as LiOH, NaOH, and the like; in a suitably selected solvent or mixture of solvents such as a mixture of THF/MeOH, 1,4-dioxane/MeOH, 2-MeTHF/MeOH, and the like; at about room temperature; to yield the corresponding compound of formula (M3b).

Compounds of formula (M2) wherein R³ is hydrogen and wherein R⁴ is methoxy or ethoxy may be prepared as described in Scheme 5 below.

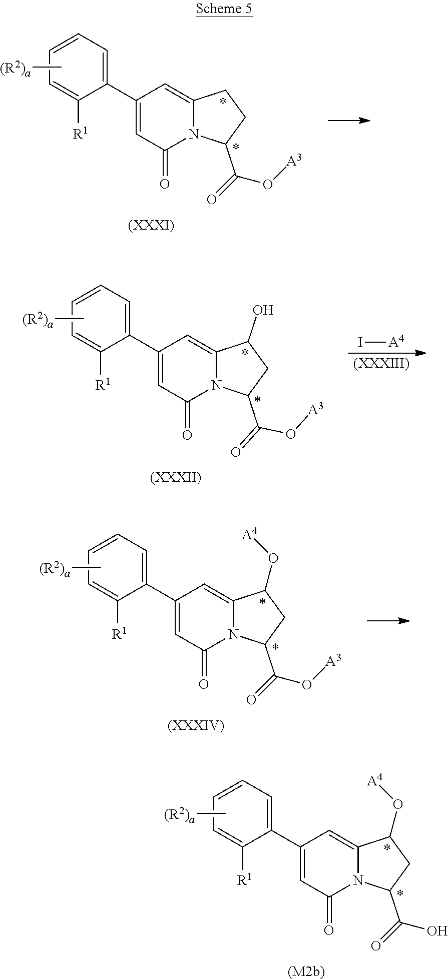

Accordingly, a suitably substituted compound of formula (XXXI), wherein A³ is C₁₋₄alkyl, preferably methyl or ethyl, a known compound, compound prepared by known methods is reacted with a suitably selected agent such as SeO₂, and the like; in a suitably selected solvent such as 1,4-dioxane, and the like; at an elevated temperature, for example at about reflux temperature; to yield the corresponding compound of formula XXXII).

The compound of formula (XXXII) is reacted with a compound of formula (XXXIII) wherein A⁴ is methyl or ethyl, a known compound or compound prepared by known methods; in the presence of a suitably selected reagent such as Ag₂O, Cs₂CO₃, and the like; in a suitably selected solvent such as DCM, acetonitrile, DCE, and the like; at an elevated temperature, for example at about reflux temperature; to yield the corresponding compound of formula (XXXIV).

The compound of formula (XXXIV) is reacted with a suitably selected acid such as TFA, HCl, and the like; in a suitably selected solvent such as DCM, 1,4-dioxane, and the like; at about room temperature; to yield the corresponding compound of formula (M2b), a compound of formula (M2) wherein R³ is hydrogen and R⁴ is methoxy or ethoxy.

Compounds of formula (M2) wherein R³ is hydrogen and R⁴ is methyl may be prepared as described in Scheme 6, below.

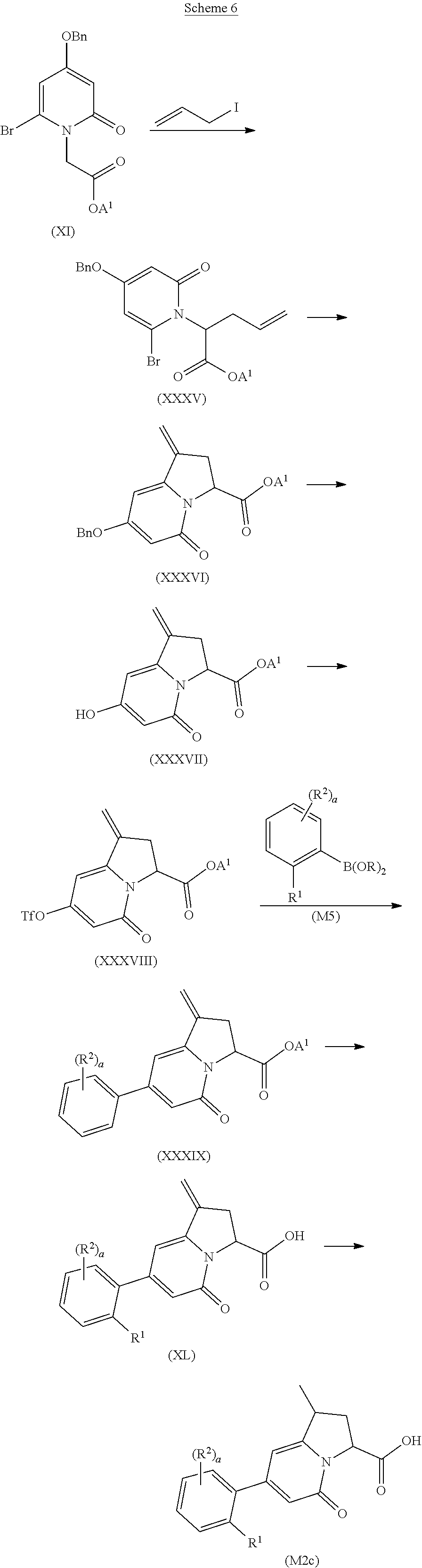

Accordingly, a suitably substituted compound of formula (XI), wherein A¹ is C₁₋₄alkyl, preferably methyl or ethyl, prepared for example as described in Scheme 2 above, is reacted with for example, 3-iodoprop-1-ene, a known compound; in the presence of a suitably selected coupling agent such as LiHMDS, KOt-Bu, and the like; in a suitably selected solvent such as THF, toluene, EtOAc, and the like; at a reduced temperature, for example at about −78° C.; to yield the corresponding compound of formula (XXXV).

The compound of formula (XXXV) is reacted with a suitably selected agent such as Pd(OAc)₂, Pd₂dba₃, and the like; in the presence of a ligand such as PPh₃, Dave-Phosp ligand, and the like; in the presence of a suitably selected organic amine base such as TEA, DIPEA, pyridine, and the like; in a suitably selected solvent such as acetonitrile, DMF, and the like; at an elevated temperature, for example at about 80° C.; to yield the corresponding compound of formula (XXXVI).

The compound of formula (XXXVI) is reacted to remove the benzyl protecting group, for example, by reacting with a suitably selected agent such as BCl₃, and the like; in a suitably selected solvent such as DCM, and the like; at a reduced temperature, for example at about −70° C.; to yield the corresponding compound of formula (XXXVII).

The compound of formula (XXXVII) is reacted with a suitably selected reagent such as 1,1,1-trifluoro-N-phenyl-N-((trifluoromethyl)sulfonyl)methane sulfonamide, and the like; in the presence of a suitably selected organic amine base such as TEA, DIPEA, pyridine, and the like; in a suitably selected solvent such as DCM, DCE, and the like; at about room temperature; to yield the corresponding compound of formula (XXXVIII).

The compound of formula (XXXVIII) is reacted with is reacted with a suitably substituted compound of formula (M5), wherein B(OR)₂ is —B(OC₁₋₄alkyl)₂ or

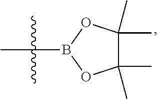 [SMILES: *B1OC(C)(C)C(C)(C)O1]

a known compound, compound prepared by known methods or compound prepared as described herein; in the presence of a suitably selected agent such as Pd(PPh₃)₄, PdCl₂dppf, Pd₂dba₃, and the like; in the presence of a suitably selected agent such as CsF, and the like; in a suitably selected solvent such as 1,4-dioxane, toluene, and the like; at an elevated temperature, for example at about reflux temperature; to yield the corresponding compound of formula (XXXIX).

The compound of formula (XXXIX) is hydrolyzed, according to known methods and depending on the identity of A¹ (for example, by reacting with a suitably selected acid such as TFA, HCl, and the like; in a suitably selected solvent such as DCM, 1,4-dioxane, and the like; at about room temperature or by reacting with a suitably selected base such as LiOH, NaOH, and the like; in a suitably selected solvent or mixture of solvents such as a mixture of THF/MeOH, 1,4-dioxane/MeOH, 2-MeTHF/MeOH, and the like); to yield the corresponding compound of formula (XL).

The compound of formula (XL) is reacted with a suitably selected reducing agent such as H_{2(g)}; in the presence of a suitably selected catalyst such as Pd/C, Pd(OH)₂, and the like; in a suitably selected solvent or mixture of solvents such as a mixture of methanol/chlorobenzene, and the lie; at about room temperature; to yield the corresponding compound of formula (M2c), a compound of formula (M2) wherein R³ is hydrogen and R⁴ is methyl.

Compounds of formula (M2) may alternatively be prepared as described in Scheme 7, below.

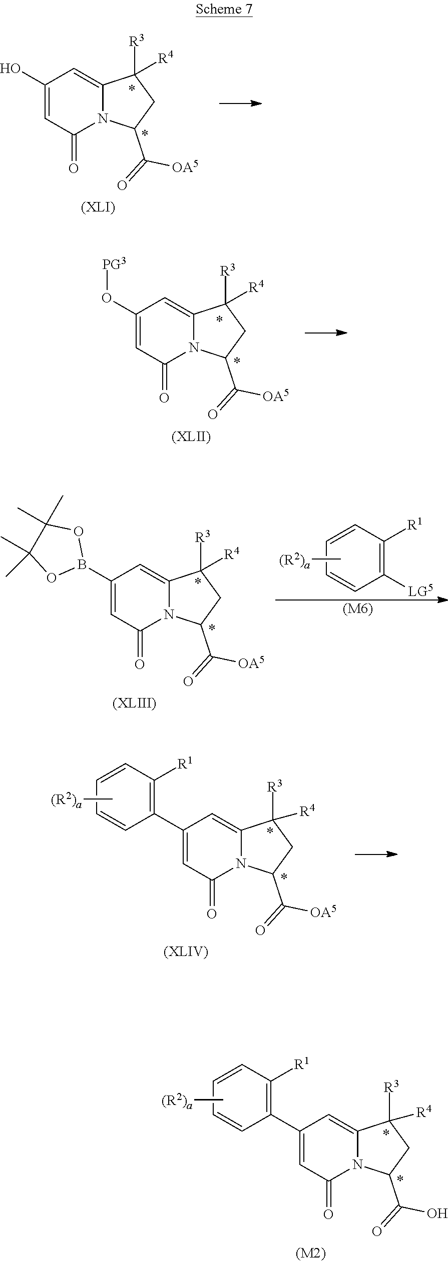

Accordingly, a suitably substituted compound of formula (XLI), wherein A⁵ is C₁₋₄alkyl, preferably methyl or ethyl, a compound prepared for example as described herein, is reacted with a suitably selected reagent such as 1,1,1-trifluoro-N-phenyl-N-((trifluoromethyl)sulfonyl)methanesulfonamide, trifluoromethanesulfonic anhydride, and the like; in the presence of a suitably selected organic amine base such as TEA, pyridine, DIEA, and the like; in a suitably selected solvent such as DCM, THF, and the like; to yield the corresponding compound of formula (XLII), wherein PG³ is the corresponding oxygen protecting group.

The compound of formula (XLII) is reacted with bis(pinacolato)diboron, a known compound; in the presence of a suitably selected coupling agent such as Pd(dppf)Cl₂, Pd(PPh₃)₄, Pd(OAc)₂, and the like; in the presence of a suitably selected base such as KOAc, potassium ethylhexanoate (n-BuCH(Et)CO₂K), and the like; in a suitably selected solvent or mixture of solvents such as a mixture of 1,4-dioxane and water, DMF, toluene/water, and the like; at an elevated temperature, such as about reflux temperature; to yield the corresponding compound of formula (XLIII).

The compound of formula (XLIII) is reacted with a suitably substituted compound of formula (M6), wherein LG⁵ is a suitably selected leaving group such as I, Br, Cl, OTf, and the like, a known compound or compound prepared by known methods; in the presence of a suitably selected coupling agent such as Pd(dppf)Cl₂, Pd(PPh₃)₄, Pd(OAc)₂, and the like; in the presence of a suitably selected base such as K₂CO₃, Cs₂CO₃, K₃PO₄, and the like; in a suitably selected solvent or mixture of solvents such as a mixture of 1,4-dioxane and water, DMF, toluene/water, and the like; at an elevated temperature, for example reflux temperature; to yield the corresponding compound of formula (XLIV).

The compound of formula (XLIV) is hydrolyzed according to known methods and depending on the identity of A¹ (for example, by reacting with a suitably selected acid such as TFA, HCl, and the like; in a suitably selected solvent such as DCM, 1,4-dioxane, and the like; at about room temperature or by reacting with a suitably selected base such as LiOH, NaOH, and the like; in a suitably selected solvent or mixture of solvents such as a mixture of THF/MeOH, 1,4-dioxane/MeOH, 2-MeTHF/MeOH, and the like), to yield the corresponding compound of formula (M2).

Compounds of formula (M3) may be similarly prepared according as described in Scheme 7, above by substituting a suitably substituted compound of formula (XLV)

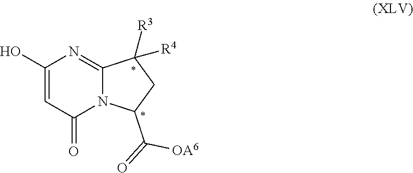

wherein A⁶ is C₁₋₄alkyl, preferably methyl or ethyl, a known compound or compound prepared by known methods, for the compound of formula (XLI), and reacting as described therein.

Compounds of formula (M2) and compounds of formula (M3) wherein R¹ is 1,2,3,4-tetrazol-1-yl may alternatively be prepared from the corresponding compound of formula (M2) or compound of formula (M3) wherein R¹ is NH₂, by reacting said compound of formula (M2) or compound of formula (M3) with a suitably selected reagent such as TMSN₃, NaN₃, and the like; in the presence of trimethoxymethane, and the like; in a suitably selected solvent such as AcOH, and the like; at an elevated temperature, for example at about 80° C.; to yield the corresponding compound of formula (M2) or compound of formula (M3) wherein R¹ is 1,2,3,4-tetrazol-1-yl.

Compounds of formula (M2) and compounds of formula (M3) wherein R¹ is for example, 4-trifluoromethyl-1,2,3-triazol-1-yl may alternatively be prepared from the corresponding compound of formula (M2) or compound of formula (M3) wherein R¹ is NH₂, by reacting said compound of formula (M2) or compound of formula (M3) with a suitably selected agent such as TMSN₃, NaN₃, and the like; in the presence of a suitably selected agent such as t-butyl nitrite, NaNO₂/HCl, and the like; in a suitably selected solvent such as acetonitrile, EtOH, heptane, and the like; at about 0° C. to room temperature; to yield the corresponding intermediate compound wherein the R¹—NH₂ group has been converted to —N₃, a compound of formula (M7) of the following structure

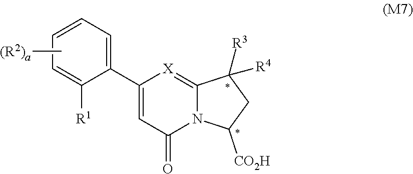

wherein X is CH (for compounds of formula (M2)) or X is N (for compounds of formula (M3)). The appropriate intermediate compound of formula (M7) is then reacted with 4,4,4-trifluorobut-2-ynoic acid, a known compound; in the presence of a suitably selected agent such as Cu₂O, CuCl, and the like; in a suitably selected solvent such as acetonitrile, DCM, and the like; at an elevated temperature, for example at about reflux temperature; to yield the corresponding compound of formula (M2) or compound of formula (M3) wherein R¹ is 4-trifluoromethyl-1,2,3-triazol-1-yl.

Compounds of formula (I) wherein

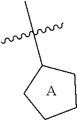

is

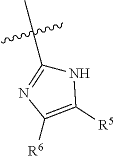

may be prepared from a suitably substituted compound of formula (M2) as described in Scheme 8, below.

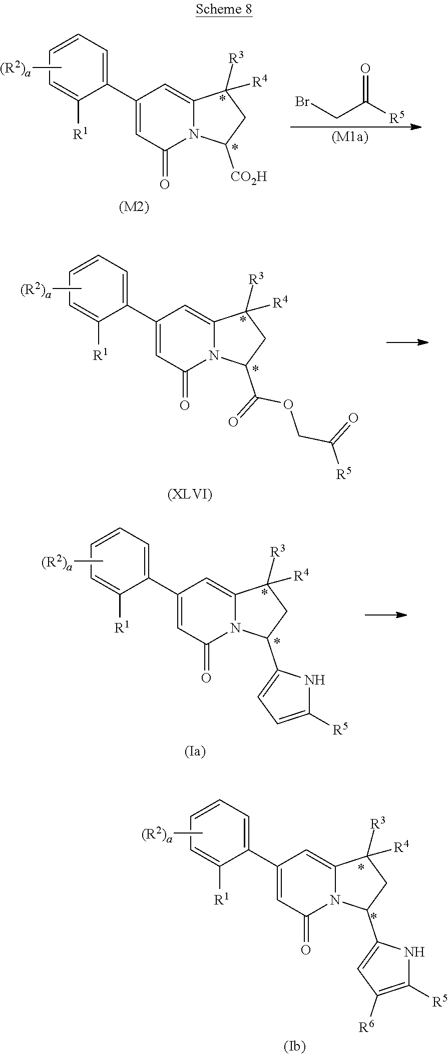

Accordingly, a suitably substituted compound of formula (M2) is reacted with a suitably substituted compound of formula (M1a), a known compound, compound prepared by known methods, or compound prepared as described in herein; in the presence of a suitably selected base such as Cs₂CO₃, K₂CO₃, Na₂CO₃, and the like; in a suitably selected solvent such as DMF, 1,4-dioxane, and the like; at about room temperature; to yield the corresponding compound of formula (XLVI).

The compound of formula (XLVI) is reacted with a suitably selected reagent such as NH₄OAc and the like; in the presence of a suitably selected acid such as AcOH, and the like; in a suitably selected solvent such as toluene, xylene, and the like; at an elevated temperature, for example at about 110° C.; to yield the corresponding compound of formula (Ia) (wherein R⁶ is hydrogen).

The compound of formula (Ia) may be further reacted with a suitably selected halogenating agent such as NCS (for chloro), NBS (for bromo), NIS (for iodo), Selectfluor (for fluoro), and the like, in a suitably selected solvent such as DCM, DCE, THF, Et₂O, and the like; to yield the corresponding compound of formula (Ib) (wherein R⁶ is the corresponding halogen).

Compounds of formula (II) wherein

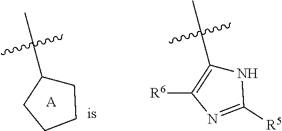

may be similarly prepared as described in Scheme X above, by substituting a suitably substituted compound of formula (M3) for the compound of formula (M2), and reacting as described therein.

Compounds of formula (I) wherein

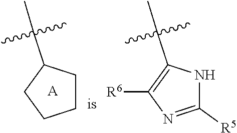

may be prepared as described in Scheme 9, below.

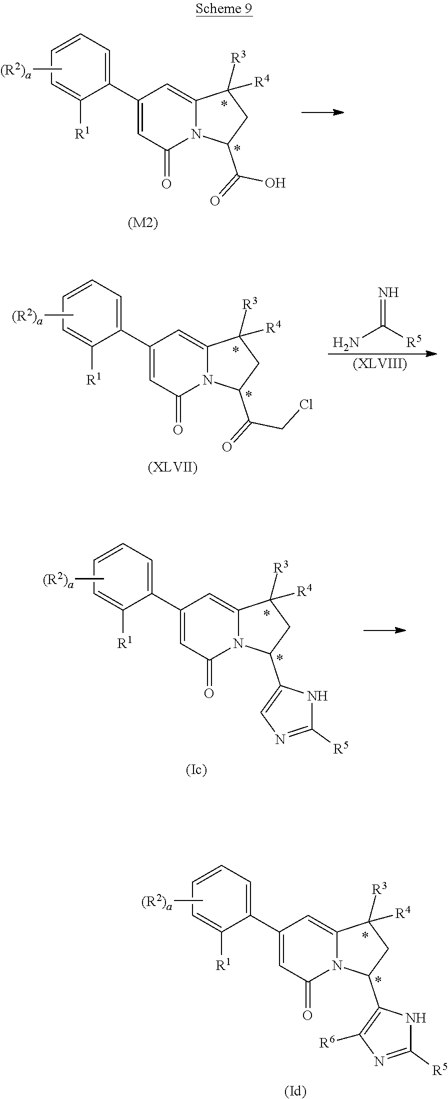

Accordingly, a suitably substituted compound of formula (M2), is reacted with oxalyl dichloride, a known compound; in a suitably selected solvent such as DCM, DCE, and the like; and then reacted with a suitably selected agent such as trimethylsilyldiazomethane (TMSCHN₂), and the like; in a suitably selected solvent such as acetonitrile, and the like; and then reacted with a suitably selected source of acid such a HCl, and the like; at a reduced temperature, for example at about 0° C.; to yield the corresponding compound of formula (XLVII).

The compound of formula (XLVII) is reacted with a suitably substituted compound of formula (XLVIII), a known compound or compound prepared by known methods, in the presence of a suitably selected base such as NaHCO₃, Cs₂CO₃, K₂CO₃, and the like; in a suitably selected solvent or mixture of solvents such as a mixture of THE/water, 1,4-dioxane/water, and the like; at an elevated temperature, for example at about 75° C.; to yield the corresponding compound of formula (Ic).

The compound of formula (Ic) may be further reacted with a suitably selected halogenating agent such as NCS (for chloro), NBS (for bromo), NIS (for iodo), Selectfluor (for fluoro), and the like, in a suitably selected solvent such as DCM, DCE, THF, Et₂O, and the like; to yield the corresponding compound of formula (Id) (wherein R⁶ is the corresponding halogen).

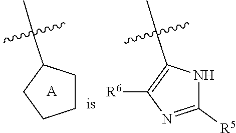

Compounds of formula (II) wherein is R may be similarly prepared as described in Scheme Y above, by substituting a suitably substituted compound of formula (M3) for the compound of formula (M2), and reacting as described therein.

Compounds of formula (I) wherein R³ is hydrogen and R⁴ is methyl may alternatively be prepared by substituting a suitably substituted compound of formula (XL), prepared for example as described in Scheme 6 above, for the compound of formula (M2) in Scheme 8 or Scheme 9 above, and reacted as described therein, to yield the corresponding compound of formula (XLVI)

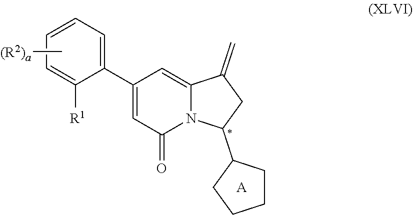

which compound of formula (XVLI) is then reacted with a suitably selected reducing agent such as H_{2(g)}; in the presence of a suitably selected catalyst such as Pd/C, Pd(OH)₂, and the like; in a suitably selected solvent or mixture of solvents such as a mixture of methanol/chlorobenzene, and the lie; at about room temperature; to yield the corresponding compound of formula (I), wherein R³ is hydrogen and R⁴ is methyl.

Compounds of formula (I) and compounds of formula (II) wherein R¹ is 1,2,3,4-tetrazol-1-yl may alternatively be prepared from the corresponding compound of formula (I) or compound of formula (II), wherein R¹ is —NH₂, by reacting with a suitably selected reagent such as TMSN₃, NaN₃, and the like; in the presence of trimethoxymethane, and the like; in a suitably selected solvent such as AcOH, and the like; at an elevated temperature, for example at about 80° C.

Compounds of formula (I) and compounds of formula (II) wherein R¹ is for example, 4-trifluoromethyl-1,2,3-triazol-1-yl may alternatively be prepared from the corresponding compound of formula (I) or compound of formula (II) wherein R¹ is NH₂, by reacting said compound of formula (I) or compound of formula (II) with a suitably selected agent such as TMSN₃, NaN₃, and the like; in the presence of a suitably selected agent such as t-butyl nitrite, NaNO₂/HCl, and the like; in a suitably selected solvent such as acetonitrile, EtOH, heptane, and the like; at about 0° C. to room temperature; to yield the corresponding intermediate wherein the R¹—NH₂ group has been converted to —N₃, a compound of formula (M8) of the following structure

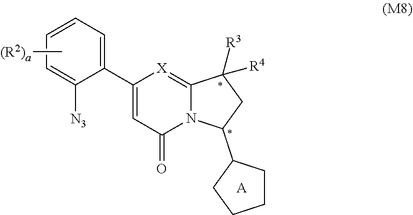

wherein X is CH (for compounds of formula (I)) and X is N (for compounds of formula (II)). The appropriate intermediate compound is then reacted with 4,4,4-trifluorobut-2-ynoic acid, a known compound; in the presence of a suitably selected agent such as Cu₂O, CuCl, and the like; in a suitably selected solvent such as acetonitrile, DCM, and the like; at an elevated temperature, for example at about reflux temperature; to yield the corresponding compound of formula (I) or compound of formula (II) wherein R1 is 4-trifluoromethyl-1,2,3-triazol-1-yl.

Compounds of formula (I) and compounds of formula (II) wherein R⁶ is halogen (for example fluoro) may be prepared from the corresponding compound of formula (I) or compound of formula (II) wherein R⁶ is hydrogen, by reacting said compound of formula (I) or compound of formula (II) wherein R⁶ is hydrogen with a suitably selected halogenating agent such as NCS (for chloro), NBS (for bromo), NIS (for iodo), Selectfluor (for fluoro), and the like, in a suitably selected solvent such as DCM, DCE, THF, Et₂O, and the like.

One skilled in the art will recognize that although the synthesis schemes above describe the preparation of racemic compounds of formula (I) and compounds of formula (II) (as well as various synthesis intermediates), the processes describe therein may be applied to the preparation of specific enantiomers, diastereomers or stereo-isomers. Said stereoisomers may be prepared by (a) using a suitably selected stereo-isomerically enriched starting material or intermediate, (b) by preparing a racemic mixture of any of the intermediates described herein, isolating the desired stereo-isomers according to known methods, for example by chiral separation, SFC, and the like, and then further reacting said stereo-isomer as described herein to yield the final compound of formula (I) or compound of formula (II) as an enriched stereo-isomer, or (c) or by preparing a racemate of the desired compound of formula (I) or compound of formula (II) and then isolating the desired stereo-isomers according to known methods, for example by chiral separation, SFC, and the like.

One skilled in the art will recognize that any substituent group(s) and/or any portion(s) of substitution group(s) (for example,

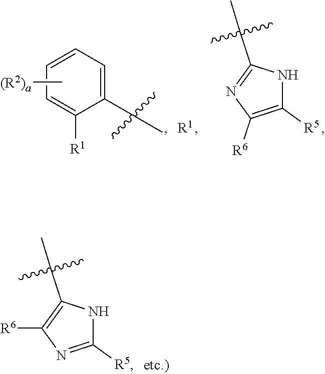

may be incorporated into the desired compound of formula (I) in any order, by applying the appropriate reaction steps in the desired order (for example, the reaction steps and conditions described in the Schemes and Examples herein).

One skilled in the art will recognize that various substituent groups and/or functional groups on said substituent groups (for example —OH, —NH₂, etc.) may be protected prior to any reaction step described above, and then de-protected at a later step in the synthesis, as would be desirable or necessary, according to methods well known to those skilled in the art.

As more extensively provided in this written description, terms such as “reacting” and “reacted” are used herein in reference to a chemical entity that is any one of: (a) the actually recited form of such chemical entity, and (b) any of the forms of such chemical entity in the medium in which the compound is being considered when named.

One skilled in the art will recognize that, where not otherwise specified, the reaction step(s) is performed under suitable conditions, according to known methods, to provide the desired product. One skilled in the art will further recognize that, in the specification and claims as presented herein, wherein a reagent or reagent class/type (e.g. base, solvent, etc.) is recited in more than one step of a process, the individual reagents are independently selected for each reaction step and may be the same of different from each other. For example, wherein two steps of a process recite an organic or inorganic base as a reagent, the organic or inorganic base selected for the first step may be the same or different than the organic or inorganic base of the second step. Further, one skilled in the art will recognize that wherein a reaction step of the present invention may be carried out in a variety of solvents or solvent systems, said reaction step may also be carried out in a mixture of the suitable solvents or solvent systems.

One skilled in the art will recognize that wherein a reaction step of the present invention may be carried out in a variety of solvents or solvent systems, said reaction step may also be carried out in a mixture of the suitable solvents or solvent systems.

One skilled in the art will further recognize that the reaction or process step(s) as herein described are allowed to proceed for a sufficient period of time until the reaction is complete, as determined by any method known to one skilled in the art, for example, chromatography (e.g. HPLC). In this context a “completed reaction or process step” shall mean that the reaction mixture contains a significantly diminished amount of the starting material(s)/reagent(s) and a significantly reduced amount of the desired product(s), as compared to the amounts of each present at the beginning of the reaction.

To provide a more concise description, some of the quantitative expressions given herein are not qualified with the term “about”. It is understood that whether the term “about” is used explicitly or not, every quantity given herein is meant to refer to the actual given value, and it is also meant to refer to the approximation to such given value that would reasonably be inferred based on the ordinary skill in the art, including approximations due to the experimental and/or measurement conditions for such given value.

To provide a more concise description, some of the quantitative expressions herein are recited as a range from about amount X to about amount Y. It is understood that wherein a range is recited, the range is not limited to the recited upper and lower bounds, but rather includes the full range from about amount X through about amount Y, or any amount or range therein.

Examples of suitable solvents, bases, reaction temperatures, and other reaction parameters and components are provided in the detailed descriptions which follow herein. One skilled in the art will recognize that the listing of said examples is not intended, and should not be construed, as limiting in any way the invention set forth in the claims which follow thereafter.

As used herein, unless otherwise noted, the term “leaving group” shall mean a charged or uncharged atom or group which departs during a substitution or displacement reaction. Suitable examples include, but are not limited to, Br, Cl, I, mesylate, tosylate, and the like.

During any of the processes for preparation of the compounds of the present invention, it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene & P. G. M. Wuts, Protective Groups in Organic Synthesis, John Wiley & Sons, 1991. The protecting groups may be removed at a convenient subsequent stage using methods known from the art.

As used herein, unless otherwise noted, the term “nitrogen protecting group” shall mean a group which may be attached to a nitrogen atom to protect said nitrogen atom from participating in a reaction and which may be readily removed following the reaction. Suitable nitrogen protecting groups include, but are not limited to carbamates—groups of the formula —C(O)O—R wherein R is for example methyl, ethyl, t-butyl, benzyl, phenylethyl, CH₂═CH—CH₂—, and the like; amides—groups of the formula —C(O)—R′ wherein R′ is for example methyl, phenyl, trifluoromethyl, and the like; N-sulfonyl derivatives—groups of the formula —SO₂—R″ wherein R″ is for example tolyl, phenyl, trifluoromethyl, 2,2,5,7,8-pentamethylchroman-6-yl-, 2,3,6-trimethyl-4-methoxybenzene, and the like. Other suitable nitrogen protecting groups may be measured in texts such as T. W. Greene & P. G. M. Wuts, Protective Groups in Organic Synthesis, John Wiley & Sons, 1991.

As used herein, unless otherwise noted, the term “oxygen protecting group” shall mean a group which may be attached to an oxygen atom to protect said oxygen atom from participating in a reaction and which may be readily removed following the reaction. Suitable oxygen protecting groups include, but are not limited to, acetyl, benzoyl, t-butyl-dimethylsilyl, trimethylsilyl (TMS), MOM, THP, and the like. Other suitable oxygen protecting groups may be measured in texts such as T. W. Greene & P. G. M. Wuts, Protective Groups in Organic Synthesis, John Wiley & Sons, 1991.

Where the processes for the preparation of the compounds according to the invention yield rise to mixture of stereoisomers, these isomers may be separated by conventional techniques such as preparative chromatography. The compounds may be prepared in racemic form, or individual enantiomers may be prepared either by enantiospecific synthesis or by resolution. The compounds may, for example, be resolved into their component enantiomers by standard techniques, such as the formation of diastereomeric pairs by salt formation with an optically active acid, such as (−)-di-p-toluoyl-D-tartaric acid and/or (+)-di-p-toluoyl-L-tartaric acid followed by fractional crystallization and regeneration of the free base. The compounds may also be resolved by formation of diastereomeric esters or amides, followed by chromatographic separation and removal of the chiral auxiliary. Alternatively, the compounds may be resolved using a chiral HPLC column.

Additionally, chiral HPLC against a standard may be used to determine percent enantiomeric excess (% ee). The enantiomeric excess may be calculated as follows

[(Rmoles−Smoles)/(Rmoles+Smoles)]×100%

where Rmoles and Smoles are the R and S mole fractions in the mixture such that Rmoles+Smoles=1. The enantiomeric excess may alternatively be calculated from the specific rotations of the desired enantiomer and the prepared mixture as follows:

ee=([α−obs]/[α−max])×100.

### 3. Utility

The compounds of the present invention are useful for the treatment and/or prophylaxis of thromboembolic disorders, inflammatory disorders and diseases or conditions in which factor XIa and/or plasma kallikrein activity is implicated.

In some embodiments, the present invention is directed to methods for the treatment and/or prophylaxis of a thromboembolic disorder comprising administering to a patient in need of such treatment and/or prophylaxis a therapeutically effective amount of a least one of the compounds as described herein, or a tautomer, stereoisomer, isotopologue, or pharmaceutically acceptable salt thereof.

As used herein, the term “thromboembolic disorders” includes arterial cardiovascular thromboembolic disorders, venous cardiovascular or cerebrovascular thromboembolic disorders, and thromboembolic disorders in the chambers of the heart or in the peripheral circulation. The term “thromboembolic disorders” as used herein also includes specific disorders selected from, but not limited to, unstable angina or other acute coronary syndromes, atrial fibrillation, first or recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from medical implants, devices, or procedures in which blood is exposed to an artificial surface that promotes thrombosis. The medical implants or devices include, but are not limited to: prosthetic valves, artificial valves, indwelling catheters, stents, blood oxygenators, shunts, vascular access ports, ventricular assist devices and artificial hearts or heart chambers, and vessel grafts. The procedures include, but are not limited to: cardiopulmonary bypass, percutaneous coronary intervention, and hemodialysis. In some embodiments, the term “thromboembolic disorders” includes acute coronary syndrome, stroke, deep vein thrombosis, and pulmonary embolism. In some embodiments, the “thromboembolic disorders” include hereditary angioedema (HAE) and diabetic macular edema (DME).

In some embodiments, the present invention is directed to methods for the treatment and/or prophylaxis of an inflammatory disorder comprising: administering to a patient in need of such treatment and/or prophylaxis a therapeutically effective amount of at least one of the compounds of the present invention or a tautomer, stereoisomer, isotopologue, or pharmaceutically acceptable salt thereof. Examples of the inflammatory disorders include, but are not limited to, sepsis, acute respiratory distress syndrome, and systemic inflammatory response syndrome.

In some embodiments, the present invention is directed to methods for the treatment and/or prophylaxis of a disease or condition in which plasma kallikrein activity is implicated, comprising administering to a patient in need of such treatment and/or prophylaxis a therapeutically effective amount of at least one of the compounds of the present invention or a tautomer, stereoisomer, isotopologue, or pharmaceutically acceptable salt thereof. The diseases or conditions in which plasma kallikrein activity is implicated include, but are not limited to, impaired visual acuity, diabetic retinopathy, diabetic macular edema, hereditary angioedema, diabetes, pancreatitis, nephropathy, cardiomyopathy, neuropathy, inflammatory bowel disease, arthritis, inflammation, septic shock, hypotension, cancer, adult respiratory distress syndrome, disseminated intravascular coagulation, and cardiopulmonary bypass surgery.

In some embodiments, the present invention provides a method for treating the primary prophylaxis of a thromboembolic disorder. In some embodiments, the present invention provides a method for the primary prophylaxis of a thromboembolic disorder wherein the thromboembolic disorder is selected from unstable angina, an acute coronary syndrome, atrial fibrillation, myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from medical implants, devices, or procedures in which blood is exposed to an artificial surface that promotes thrombosis. In another embodiment, the present invention provides a method for the primary prophylaxis of a thromboembolic disorder, wherein the thromboembolic disorder is selected from acute coronary syndrome, stroke, venous thrombosis, and thrombosis resulting from medical implants and devices.

In some embodiments, the present invention provides a method for the secondary prophylaxis of a thromboembolic disorder. In some embodiments, the present invention provides a method for the secondary prophylaxis of a thromboembolic disorder. wherein the thromboembolic disorder is selected from unstable angina, an acute coronary syndrome, atrial fibrillation, recurrent myocardial infarction, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from medical implants, devices, or procedures in which blood is exposed to an artificial surface that promotes thrombosis. In another embodiment, the present invention provides a method for the secondary prophylaxis of a thromboembolic disorder, wherein the thromboembolic disorder is selected from acute coronary syndrome, stroke, atrial fibrillation and venous thrombosis.

One skilled in the art will recognize that wherein the present invention is directed to methods of prophylaxis, the subject in need thereof (i.e. a subject in need of prophylaxis) shall include any subject or patient (preferably a mammal, more preferably a human) who has experienced or exhibited at least one symptom of the disorder, disease or condition to be prevented. Further, a subject in need thereof may additionally be a subject (preferably a mammal, more preferably a human) who has not exhibited any symptoms of the disorder, disease or condition to be prevented, but who has been deemed by a physician, clinician or other medical profession to be at risk of developing said disorder, disease or condition. For example, the subject may be deemed at risk of developing a disorder, disease or condition (and therefore in need of prophylaxis or prophylactic treatment) as a consequence of the subject's medical history, including, but not limited to, family history, pre-disposition, co-existing (comorbid) disorders or conditions, genetic testing, and the like.

The compounds of the present invention are preferably administered alone to a mammal in a therapeutically effective amount. However, the compounds of the invention can also be administered in combination with an additional therapeutic agent, as defined below, to a mammal in a therapeutically effective amount. When administered in a combination, the combination of compounds is preferably, but not necessarily, a synergistic combination. Synergy, for example, may occur when the effect (in this case, inhibition of the desired target) of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased anticoagulant effect, or some other beneficial effect of the combination compared with the individual components. Possible favorable outcomes of treatment with a synergistic combination include, but are not limited to, (a) increased efficacy of the therapeutic effect, (b) ability to administer decreased dosage while increasing or maintaining efficacy (which in turn may also result in decreased toxicity and/or adverse side effects), (c) minimized or slowed development of drug resistance, (d) selective synergism against the biological target (or efficacy synergism) versus host (toxicity antagonism).

In some embodiments of the present invention, the compound of formula (I), compound of formula (II), compound of formula (III) or compound of formula (IV) may be administered in combination with one or more anticoagulant, anti-thrombin agent, anti-platelet agent, fibrinolytic, hypolipidemic agent, antihypertensive agent, and/or anti-ischemic agent. Suitable examples include, but are not limited to warfarin, heparin, aprotinin, a synthetic pentasaccharide, a boroarginine derivative, a boropeptide, heparin, hirudin, argatroban, a thromboxane-A2-receptor antagonist, a thromboxane-A2-synthetase inhibitor, a PDE-III inhibitor, a PDE V inhibitor, an ADP receptor antagonist, an antagonist of the purinergic receptor P2Y1, an antagonist of the purinergic receptor P2Y12, tissue plasminogen activator and modified forms thereof, anistreplase, urokinase, streptokinase, tenecteplase, lanoteplase, a PAI-I inhibitor, an alpha-2-antiplasmin inhibitor, an anisoylated plasminogen streptokinase activator complex, a HMG-CoA reductase inhibitor, a squalene synthetase inhibitor, a fibrate, a bile acid sequestrant, an ACAT inhibitor, a MTP inhibitor, a lipooxygenase inhibitor, a cholesterol absorption inhibitor, a cholesterol ester transfer protein inhibitor, an alpha adrenergic blocker, a beta adrenergic blocker, a calcium channel blocker, a diuretic, a renin inhibitor, an angiotensin-converting enzyme inhibitor, an angiotensin-II-receptor antagonist, an ET receptor antagonist, a Dual ET/A11 antagonist, a neutral endopeptidase inhibitor, a vasopeptidase inhibitor, a Class I agent, a Class II agent, a Class III agent, a Class IV agent, an IAch inhibitor, an IKur inhibitor and a cardiac glycoside.

By “administered in combination” or “combination therapy” it is meant that the compound of the present invention and one or more additional therapeutic agents are administered concurrently or consecutively to the subject (preferably mammal, more preferably human) being treated. When administered in combination each component may be administered at the same time or sequentially in any order at different points in time. Thus, each component may be administered separately but sufficiently closely in time so as to provide the desired therapeutic effect. Chou, Theoretical Basis, Experimental Design, and Computerized Simulation of Synergism and Antagonism in Drug Combination Studies, Pharmacol Rev., 2006, vol. 58, 621-681.

### 4. Pharmaceutical Compositions

The present invention further comprises pharmaceutical compositions containing a compound of formula (I), compound of formula (II), compound of formula (III) and/or compound of formula (IV) with a pharmaceutically acceptable carrier. Pharmaceutical compositions containing one or more of the compounds of the invention described herein as the active ingredient can be prepared by intimately mixing the compound or compounds with a pharmaceutical carrier according to conventional pharmaceutical compounding techniques. The carrier may take a wide variety of forms depending upon the desired route of administration (e.g., oral, parenteral). Thus, for liquid oral preparations such as suspensions, elixirs and solutions, suitable carriers and additives include water, glycols, oils, alcohols, flavoring agents, preservatives, stabilizers, coloring agents and the like; for solid oral preparations, such as powders, capsules and tablets, suitable carriers and additives include starches, sugars, diluents, granulating agents, lubricants, binders, disintegrating agents and the like. Solid oral preparations may also be coated with substances such as sugars or be enteric-coated so as to modulate major site of absorption. For parenteral administration, the carrier will usually consist of sterile water and other ingredients may be added to increase solubility or preservation. Injectable suspensions or solutions may also be prepared utilizing aqueous carriers along with appropriate additives.

To prepare the pharmaceutical compositions of this invention, one or more compounds of the present invention as the active ingredient is intimately admixed with a pharmaceutical carrier according to conventional pharmaceutical compounding techniques, which carrier may take a wide variety of forms depending on the form of preparation desired for administration, e.g., oral or parenteral such as intramuscular. In preparing the compositions in oral dosage form, any of the usual pharmaceutical media may be employed. Thus, for liquid oral preparations, such as for example, suspensions, elixirs and solutions, suitable carriers and additives include water, glycols, oils, alcohols, flavoring agents, preservatives, coloring agents and the like; for solid oral preparations such as, for example, powders, capsules, caplets, gelcaps and tablets, suitable carriers and additives include starches, sugars, diluents, granulating agents, lubricants, binders, disintegrating agents and the like. Because of their ease in administration, tablets and capsules represent the most advantageous oral dosage unit form, in which case solid pharmaceutical carriers are obviously employed. If desired, tablets may be sugar coated or enteric coated by standard techniques. For parenteral, the carrier will usually comprise sterile water, through other ingredients, for example, for purposes such as aiding solubility or for preservation, may be included. Injectable suspensions may also be prepared, in which case appropriate liquid carriers, suspending agents and the like may be employed. The pharmaceutical compositions herein will contain, per dosage unit, e.g., tablet, capsule, powder, injection, teaspoonful and the like, an amount of the active ingredient necessary to deliver an effective dose as described above. The pharmaceutical compositions herein will contain, per unit dosage unit, e.g., tablet, capsule, powder, injection, suppository, teaspoonful and the like, of from about 0.01 mg to about 1000 mg or any amount or range therein, and may be given at a dosage of from about 0.05 mg/day to about 1000 mg/day, or any amount or range therein, about 0.1 mg/day to about 500 mg/day, or any amount or range therein, preferably from about 1 mg/day to about 300 mg/day, or any amount or range therein.

The dosages, however, may be varied depending upon the requirement of the patients, the severity of the condition being treated and the compound being employed. The use of either daily administration or post-periodic dosing may be employed.

Preferably these compositions are in unit dosage forms such as tablets, pills, capsules, powders, granules, sterile parenteral solutions or suspensions, metered aerosol or liquid sprays, drops, ampoules, autoinjector devices or suppositories; for oral parenteral, intranasal, sublingual or rectal administration, or for administration by inhalation or insufflation. Alternatively, the composition may be presented in a form suitable for once-weekly or once-monthly administration; for example, an insoluble salt of the active compound, such as the decanoate salt, may be adapted to provide a depot preparation for intramuscular injection. For preparing solid compositions such as tablets, the principal active ingredient is mixed with a pharmaceutical carrier, e.g. conventional tableting ingredients such as corn starch, lactose, sucrose, sorbitol, talc, stearic acid, magnesium stearate, dicalcium phosphate or gums, and other pharmaceutical diluents, e.g. water, to form a solid pre-formulation composition containing a homogeneous mixture of a compound of the present invention, or a pharmaceutically acceptable salt thereof. When referring to these pre-formulation compositions as homogeneous, it is meant that the active ingredient is dispersed evenly throughout the composition so that the composition may be readily subdivided into equally effective dosage forms such as tablets, pills and capsules. This solid pre-formulation composition is then subdivided into unit dosage forms of the type described above containing from about 0.01 mg to about 1,000 mg, or any amount or range therein, of the active ingredient of the present invention. The tablets or pills of the novel composition can be coated or otherwise compounded to provide a dosage form yielding the advantage of prolonged action. For example, the tablet or pill can comprise an inner dosage and an outer dosage component, the latter being in the form of an envelope over the former. The two components can be separated by an enteric layer which serves to resist disintegration in the stomach and permits the inner component to pass intact into the duodenum or to be delayed in release. A variety of material can be used for such enteric layers or coatings, such materials including a number of polymeric acids with such materials as shellac, cetyl alcohol and cellulose acetate.

The liquid forms in which the novel compositions of the present invention may be incorporated for administration orally or by injection include, aqueous solutions, suitably flavored syrups, aqueous or oil suspensions, and flavored emulsions with edible oils such as cottonseed oil, sesame oil, coconut oil or peanut oil, as well as elixirs and similar pharmaceutical vehicles. Suitable dispersing or suspending agents for aqueous suspensions, include synthetic and natural gums such as tragacanth, acacia, alginate, dextran, sodium carboxymethylcellulose, methylcellulose, polyvinyl-pyrrolidone or gelatin.

The method of the treatment and/or prophylaxis of thromboembolic disorders described in the present invention may also be carried out using a pharmaceutical composition comprising any of the compounds as defined herein and a pharmaceutically acceptable carrier. The pharmaceutical composition may contain between about 0.01 mg and about 1000 mg of the compound, or any amount or range therein, preferably from about 0.05 mg to about 300 mg of the compound, or any amount or range therein, more preferably from about 0.1 mg to about 100 mg of the compound, or any amount or range therein, more preferably from about 0.1 mg to about 50 mg of the compound, or any amount or range therein; and may be constituted into any form suitable for the mode of administration selected. Carriers include necessary and inert pharmaceutical excipients, including, but not limited to, binders, suspending agents, lubricants, flavorants, sweeteners, preservatives, dyes, and coatings. Compositions suitable for oral administration include solid forms, such as pills, tablets, caplets, capsules (each including immediate release, timed release and sustained release formulations), granules, and powders, and liquid forms, such as solutions, syrups, elixirs, emulsions, and suspensions. Forms useful for parenteral administration include sterile solutions, emulsions and suspensions.

Advantageously, compounds of the present invention may be administered in a single daily dose, or the total daily dosage may be administered in divided doses of two, three or four times daily. Furthermore, compounds for the present invention can be administered in intranasal form via topical use of suitable intranasal vehicles, or via transdermal skin patches well known to those of ordinary skill in that art. To be administered in the form of a transdermal delivery system, the dosage administration will, of course, be continuous rather than intermittent throughout the dosage regimen.

For instance, for oral administration in the form of a tablet or capsule, the active drug component can be combined with an oral, non-toxic pharmaceutically acceptable inert carrier such as ethanol, glycerol, water and the like. Moreover, when desired or necessary, suitable binders; lubricants, disintegrating agents and coloring agents can also be incorporated into the mixture. Suitable binders include, without limitation, starch, gelatin, natural sugars such as glucose or beta-lactose, corn sweeteners, natural and synthetic gums such as acacia, tragacanth or sodium oleate, sodium stearate, magnesium stearate, sodium benzoate, sodium acetate, sodium chloride and the like. Disintegrators include, without limitation, starch, methyl cellulose, agar, bentonite, xanthan gum and the like.

The liquid forms may include suitably flavored suspending or dispersing agents such as the synthetic and natural gums, for example, tragacanth, acacia, methyl-cellulose and the like. For parenteral administration, sterile suspensions and solutions are desired. Isotonic preparations which generally contain suitable preservatives are employed when intravenous administration is desired.

To prepare a pharmaceutical composition of the present invention, a compound of formula (I), compound of formula (II), compound of formula (III), and/or compound of formula (IV) as the active ingredient is intimately admixed with a pharmaceutical carrier according to conventional pharmaceutical compounding techniques, which carrier may take a wide variety of forms depending of the form of preparation desired for administration (e.g. oral or parenteral). Suitable pharmaceutically acceptable carriers are well known in the art. Descriptions of some of these pharmaceutically acceptable carriers may be measured in The Handbook of Pharmaceutical Excipients, published by the American Pharmaceutical Association and the Pharmaceutical Society of Great Britain. Methods of formulating pharmaceutical compositions have been described in numerous publications such as Pharmaceutical Dosage Forms: Tablets, Second Edition, Revised and Expanded, Volumes 1-3, edited by Lieberman et al; Pharmaceutical Dosage Forms: Parenteral Medications, Volumes 1-2, edited by Avis et al; and Pharmaceutical Dosage Forms: Disperse Systems, Volumes 1-2, edited by Lieberman et al; published by Marcel Dekker, Inc.

Compounds of the present invention may be administered in any of the foregoing compositions and according to dosage regimens established in the art whenever treatment or prophylaxis of thromboembolic disorders, inflammatory disorders or diseases or conditions in which plasma kallikrein activity is implicated is required.

The daily dosage of the products may be varied over a wide range from about 0.01 mg to about 1,000 mg per adult human per day, or any amount or range therein. For oral administration, the compositions are preferably provided in the form of tablets containing, 0.01, 0.05, 0.1, 0.5, 1.0, 2.5, 5.0, 10.0, 15.0, 25.0, 50.0, 100, 150, 200, 250 and 500 milligrams of the active ingredient for the symptomatic adjustment of the dosage to the patient to be treated. An effective amount of the drug may be ordinarily supplied at a dosage level of from about 0.005 mg/kg to about 10 mg/kg of body weight per day, or any amount or range therein. Preferably, the range is from about 0.01 to about 5.0 mg/kg of body weight per day, or any amount or range therein, more preferably, from about 0.1 to about 1.0 mg/kg of body weight per day, or any amount or range therein, more preferably, from about 0.1 to about 0.5 mg/kg of body weight per day, or any amount or range therein. The compounds may be administered on a regimen of 1 to 4 times per day.

Optimal dosages to be administered may be readily determined by those skilled in the art, and will vary with the particular compound used, the mode of administration, the strength of the preparation, and the advancement of the disease condition. In addition, factors associated with the particular patient being treated, including patient age, weight, diet and time of administration, will result in the need to adjust dosages.

One skilled in the art will recognize that, both in vivo and in vitro trials using suitable, known and generally accepted cell and/or animal models are predictive of the ability of a test compound to treat or prevent a given disorder.

One skilled in the art will further recognize that human clinical trials including first-in-human, dose ranging and efficacy trials, in healthy patients and/or those suffering from a given disorder, may be completed according to methods well known in the clinical and medical arts.

### 5. Combination Therapy

One or more additional pharmacologically active agents may be administered in combination with the compounds of the invention. The additional active agent (or agents) is intended to mean a pharmaceutically active agent (or agents) that is active in the body, including pro-drugs that convert to pharmaceutically active form after administration, which is different from the compound of formula (I), compound of formula (II), compound of formula (III) and/or compound of formula (IV) and also includes free-acid, free-base and pharmaceutically acceptable salts of said additional active agents when such forms are sold commercially or are otherwise chemically possible. Generally, any suitable additional active agent or agents, including but not limited to anti-hypertensive agents, additional diuretics, anti-atherosclerotic agents such as a lipid modifying compound, anti-diabetic agents and/or anti-obesity agents may be used in any combination with the compound of formula (I), compound of formula (II), compound of formula (III) and/or compound of formula (IV) in a single dosage formulation (a fixed dose drug combination), or may be administered to the patient in one or more separate dosage formulations which allows for concurrent or sequential administration of the active agents (co-administration of the separate active agents).

Examples of additional active agents which may be employed include but are not limited to angiotensin converting enzyme inhibitors (e.g., alacepril, benazepril, captopril, ceronapril, cilazapril, delapril, enalapril, enalaprilat, fosinopril, imidapril, lisinopril, moveltipril, perindopril, quinapril, ramipril, spirapril, temocapril, or trandolapril); angiotensin II receptor antagonists also known as angiotensin receptor blockers or ARBs (e.g., losartan i.e., COZAAR®, valsartan, candesartan, olmesartan, telmesartan, eprosartan, irbesartan and any of these drugs used in combination with hydrochlorothiazide such as HYZAAR®); diuretics, e.g. hydrochlorothiazide (HCTZ); potassium sparing diuretics such as amiloride HCl, spironolactone, epleranone, triamterene, each with or without HCTZ; neutral endopeptidase inhibitors (e.g., thiorphan and phosphoramidon); aldosterone antagonists; aldosterone synthase inhibitors; renin inhibitors (e.g. urea derivatives of di- and tri-peptides (See U.S. Pat. No. 5,116,835), amino acids and derivatives (U.S. Pat. Nos. 5,095,119 and 5,104,869), amino acid chains linked by non-peptidic bonds (U.S. Pat. No. 5,114,937), di- and tri-peptide derivatives (U.S. Pat. No. 5,106,835), peptidyl amino diols (U.S. Pat. Nos. 5,063,208 and 4,845,079) and peptidyl beta-aminoacyl aminodiol carbamates (U.S. Pat. No. 5,089,471); also, a variety of other peptide analogs as disclosed in the following U.S. Pat. Nos. 5,071,837; 5,064,965; 5,063,207; 5,036,054; 5,036,053; 5,034,512 and 4,894,437, and small molecule renin inhibitors (including diol sulfonamides and sulfinyls (U.S. Pat. No. 5,098,924), N-morpholino derivatives (U.S. Pat. No. 5,055,466), N-heterocyclic alcohols (U.S. Pat. No. 4,885,292) and pyrrolimidazolones (U.S. Pat. No. 5,075,451); also, pepstatin derivatives (U.S. Pat. No. 4,980,283) and fluoro- and chloro-derivatives of statone-containing peptides (U.S. Pat. No. 5,066,643); enalkrein; RO 42-5892; A 65317; CP 80794; ES 1005; ES 8891; SQ 34017; aliskiren (2(S),4(S),5(S),7(S)—N-(2-carbamoyl-2-methylpropyl)-5-amino-4-hydroxy-2,7-diisopropyl-8-[4-methoxy-3-(3-methoxypropoxy)-phenyl]-octanamide hemifumarate) SPP600, SPP630 and SPP635); endothelin receptor antagonists; vasodilators (e.g. nitroprusside); calcium channel blockers (e.g., amlodipine, nifedipine, verapamil, diltiazem, felodipine, gallopamil, niludipine, nimodipine, nicardipine); potassium channel activators (e.g., nicorandil, pinacidil, cromakalim, minoxidil, aprilkalim, loprazolam); sympatholitics; beta-adrenergic blocking drugs (e.g., acebutolol, atenolol, betaxolol, bisoprolol, carvedilol, metoprolol, metoprolol tartate, nadolol, propranolol, sotalol, timolol); alpha adrenergic blocking drugs (e.g., doxazocin, prazocin or alpha methyldopa); central alpha adrenergic agonists; peripheral vasodilators (e.g. hydralazine); lipid lowering agents, e.g., HMG-CoA reductase inhibitors such as simvastatin and lovastatin which are marketed as ZOCOR® and MEVACOR® in lactone pro-drug form and function as inhibitors after administration, and pharmaceutically acceptable salts of dihydroxy open ring acid HMG-CoA reductase inhibitors such as atorvastatin (particularly the calcium salt sold in LIPITOR®), rosuvastatin (particularly the calcium salt sold in CRESTOR®), pravastatin (particularly the sodium salt sold in PRAVACHOL®), and fluvastatin (particularly the sodium salt sold in LESCOL®); a cholesterol absorption inhibitor such as ezetimibe (ZETIA®), and ezetimibe in combination with any other lipid lowering agents such as the HMG-CoA reductase inhibitors noted above and particularly with simvastatin (VYTORIN®) or with atorvastatin calcium; niacin in immediate-release or controlled release forms, and particularly niacin in combination with a DP antagonist such as laropiprant (TREDAPTIVE®) and/or with an HMG-CoA reductase inhibitor; niacin in immediate-release or controlled release forms, and particularly niacin in combination with a DP antagonist such as laropiprant (TREDAPTIVE®) and/or with an HMG-CoA reductase inhibitor; niacin receptor agonists such as acipimox and acifran, as well as niacin receptor partial agonists; metabolic altering agents including insulin sensitizing agents and related compounds for the treatment of diabetes such as biguanides (e.g., metformin), meglitinides (e.g., repaglinide, nateglinide), sulfonylureas (e.g., chlorpropamide, glimepiride, glipizide, glyburide, tolazamide, tolbutamide), thiazolidinediones also referred to as glitazones (e.g., pioglitazone, rosiglitazone), alpha glucosidase inhibitors (e.g., acarbose, miglitol), dipeptidyl peptidase inhibitors, (e.g., sitagliptin (JANUVIA®), alogliptin, vildagliptin, saxagliptin, linagliptin, dutogliptin, gemigliptin), ergot alkaloids (e.g., bromocriptine), combination medications such as JANUMET® (sitagliptin with metformin), and injectable diabetes medications such as exenatide and pramlintide acetate; or with other drugs beneficial for the prevention or the treatment of the above-mentioned diseases including but not limited to diazoxide; and including the free-acid, free-base, and pharmaceutically acceptable salt forms of the above active agents where chemically possible. Compounds which can be alternatively or additionally administered in combination with the compounds of the present invention include, but are not limited to, anticoagulants, anti-thrombin agents, anti-platelet agents, fibrinolytics, hypolipidemic agents, antihypertensive agents, and anti-ischemic agents.

Anticoagulant agents (or coagulation inhibitory agents) that may be used in combination with the compounds of this invention include warfarin, heparin (either unfractionated heparin or any commercially available low molecular weight heparin, for example enoxaparin and dalteparin), aprotinin, synthetic pentasaccharide inhibitors of Factor Xa such as fondaparinux and idraparinux, direct Factor Xa inhibitors such as rivaroxaban, apixaban, betrixaban, edoxaban, otamixaban, direct acting thrombin inhibitors including hirudin, dabigatran, argatroban, ximelagatran, melagatran, lepirudin, desirudin, and bivalirudin, as well as other factor VIIa inhibitors, VIIIa inhibitors, DCa inhibitors, Xa inhibitors, XIa inhibitors, fibrinogen receptor antagonists (including abciximab, eptifibatide and tirofiban), TAFI inhibitors, and others known in the art. Factor DCa inhibitors include synthetic active-site blocked competitive inhibitors, oral inhibitors and RNA aptamers. These are described in Howard, E L, Becker K C, Rusconi, C P, Becker R C. Factor IXa Inhibitors as Novel Anticoagulents. Arterioscler Thromb Vasc Biol, 2007; 27: 722-727.

The term “anti-platelet agents” or “platelet inhibitory agents”, as used herein, denotes agents that inhibit platelet function, for example, by inhibiting the aggregation, adhesion or granular secretion of platelets. Such agents include, but are not limited to, the various known non-steroidal anti-inflammatory drugs (NSAIDS) such as aspirin, ibuprofen, naproxen, sulindac, indomethacin, mefenamate, droxicam, diclofenac, sulfinpyrazone, and piroxicam, including pharmaceutically acceptable salts or prodrugs thereof. Of the NSAIDS, aspirin (acetylsalicylic acid or ASA), and piroxicam are preferred. Other suitable platelet inhibitory agents include IIb/IIIa antagonists (e.g., tirofiban, eptifibatide, and abciximab), thromboxane-A2-receptor antagonists (e.g., ifetroban), thromboxane-A2-synthetase inhibitors, phosphodiesterase-III (PDE-III) inhibitors (e.g., dipyridamole, cilostazol), and PDE V inhibitors (such as sildenafil), and pharmaceutically acceptable salts or prodrugs thereof.

The term “anti-platelet agents” or “platelet inhibitory agents”, as used herein, is also intended to include ADP (adenosine diphosphate) receptor antagonists, preferable antagonists of the purinergic receptors P2Y1 and P2Y12 with P2Y12 being even more preferred. Preferred P2Y12 receptor antagonists include ticlopidine, prasugrel, clopidogrel, elinogrel, ticagrelor and cangrelor, including pharmaceutically acceptable salts or prodrugs thereof. Clopidogrel is an even more preferred agent. Ticlopidine and clopidogrel are also preferred compounds since they are known to be gentle on the gastro-intestinal tract in use. The compounds of the present invention may also be dosed in combination with aprotinin.

The term “thrombin inhibitors” or “anti-thrombin agents”, as used herein, denotes inhibitors of the serine protease thrombin. By inhibiting thrombin, various thrombin-mediated processes, such as thrombin-mediated platelet activation (that is, for example, the aggregation of platelets, and/or the granular secretion of plasminogen activator inhibitor-I and/or serotonin), endothelial cell activation, inflammatory reactions, and/or fibrin formation are disrupted. A number of thrombin inhibitors are known to one of skill in the art and these inhibitors are contemplated to be used in combination with the present compounds. Such inhibitors include, but are not limited to, boroarginine derivatives, boropeptides, heparins, hirudin, dabigatran and argatroban, including pharmaceutically acceptable salts and prodrugs thereof. Boroarginine derivatives and boropeptides include N-acetyl and peptide derivatives of boronic acid, such as C-terminal alpha-aminoboronic acid derivatives of lysine, ornithine, arginine, homoarginine and corresponding isothiouronium analogs thereof. The term “hirudin”, as used herein, includes suitable derivatives or analogs of hirudin, referred to herein as hirulogs, such as disulfatohirudin.

The term “thrombin receptor antagonists”, also known as protease activated receptor (PAR) antagonists or PAR-1 antagonists, are useful in the treatment of thrombotic, inflammatory, atherosclerotic and fibroproliferative disorders, as well as other disorders in which thrombin and its receptor play a pathological role. Thrombin receptor antagonist peptides have been identified based on structure-activity studies involving substitutions of amino acids on thrombin receptors. In Bernatowicz et al, J Med. Chem., vol. 39, pp. 4879-4887 (1996), tetra- and pentapeptides are disclosed as being potent thrombin receptor antagonists, for example N-trans-cinnamoyl-p-fluoroPhe-p-guanidinoPhe-Leu-Arg-NH₂ and N-trans-cinnamoyl-p-fluoroPhe-p-guanidinoPhe-Leu-Arg-Arg-NH₂. Peptide thrombin receptor antagonists are also disclosed in WO 94/03479. Substituted tricyclic thrombin receptor antagonists are disclosed in U.S. Pat. Nos. 6,063,847, 6,326,380 and WO 01/96330. Other thrombin receptor antagonists include those disclosed in U.S. Pat. Nos. 7,304,078; 7,235,567; 7,037,920; 6,645,987; and EP Patent Nos. EP1495018 and EP1294714.

The term thrombolytic (or fibrinolytic) agents (or thrombolytics or fibrinolytics), as used herein, denotes agents that lyse blood clots (thrombi). Such agents include tissue plasminogen activator (TPA, natural or recombinant) and modified forms thereof, anistreplase, urokinase, streptokinase, tenecteplase (TNK), lanoteplase (nPA), factor VIIa inhibitors, PAI-I inhibitors (i.e., inactivators of tissue plasminogen activator inhibitors), alpha-2-antiplasmin inhibitors, and anisoylated plasminogen streptokinase activator complexes, including pharmaceutically acceptable salts or prodrugs thereof. The term anistreplase, as used herein, refers to anisoylated plasminogen streptokinase activator complexes. The term urokinase, as used herein, is intended to denote both dual and single chain urokinase, the latter also being referred to herein as prourokinase. Examples of suitable anti-arrhythmic agents for use in combination with the present compounds include: Class I agents (such as propafenone); Class II agents (such as carvedilol and propranolol); Class III agents (such as sotalol, dofetilide, aminodarone, azimilide and ibutilide); Class IV agents (such as ditiazem and verapamil); IAch inhibitors, and IKur inhibitors (e.g., compounds such as those disclosed in WO01/40231).

### 6. Definitions

As used herein, unless otherwise noted, “halogen” shall mean chloro, bromo, fluoro and iodo, preferably bromo, fluoro or chloro, more preferably fluoro or chloro.

As used herein, unless otherwise noted, the term “oxo” shall mean a functional group of the structure ═O (i.e. a substituent oxygen atom connected to another atom by a double bond).

As used herein, unless otherwise noted, the term “C_{X-Y}alkyl” wherein X and Y are integers, whether used alone or as part of a substituent group, include straight and branched chains containing between X and Y carbon atoms. For example, C₁₋₄alkyl radicals include straight and branched chains of between 1 and 4 carbon atoms, including methyl, ethyl, propyl, isopropyl, butyl, isobutyl, sec-butyl and t-butyl.

As used herein, unless otherwise noted, the terms “—(C_{X-Y}alkylene)- and —C_{X-Y}alkylene-” wherein X and Y are integers, shall denote any C_{X-Y}alkyl carbon chain as herein defined, wherein said C_{X-Y}alkyl chain is divalent and is further bound through two points of attachment, preferably through two terminal carbon atoms.

As used herein, unless otherwise noted, the term “hydroxy substituted C₁₋₄alkyl” shall mean any C₁₋₄alkyl group as defined above substituted with at least one hydroxy (—OH) groups, preferably one to three, more preferably one to two hydroxy groups. Suitable examples include but are not limited to —CH₂OH, —CH₂CH₂OH, —CH(OH)CH₃, —CH(OH)CH₂OH, —CH₂CH₂CH₂OH, —C(CH₂OH)₃, and the like.

As used herein, unless otherwise noted, the term “fluorinated C₁₋₄alkyl” shall mean any C₁₋₄alkyl group as defined above substituted with one or more fluoro groups, preferably one to three fluoro group. Suitably examples include, but are not limited to —CH₂F, —CHF₂, —CF₃, —CH₂—CF₃, —CF₂—CH₃, —CH₂—CH₂—CH₂F, —CH₂—CH₂—CF₃, —C(CH₃)₂CF₃, —C(CF₃)₃, and the like.

As used herein, unless otherwise noted, the term “hydroxy substituted fluorinated C₁₋₄alkyl” shall mean any C₁₋₄alkyl group as defined above, wherein the C₁₋₄alkyl group is substituted with one or more, preferably one to three, preferably one, hydroxy group(s) and further substituted with one or more, preferably one to six, preferably one to three, fluoro group(s). Suitably examples include but are not limited to —CF₂(OH), —CH(OH)—CF₃, —CH₂—CH(OH)—CF₃, —CH₂—CH₂—CH(OH)—CF₃, and the like.

As used herein, unless otherwise noted, “C_{X-Y}alkoxy” wherein X and Y are integers, shall mean an oxygen ether radical of the above described straight or branched chain C_{X-Y}alkyl groups containing between X and Y carbon atoms. For example, C₁₋₄alkoxy shall include methoxy, ethoxy, n-propoxy, isopropoxy, n-butyloxy, iso-butyloxy, sec-butyloxy and tert-butyloxy.

As used herein, unless otherwise noted, the term “hydroxy substituted C₁₋₄alkoxy” shall mean any C₁₋₄alkoxy group as defined above substituted with at least one hydroxy (—OH) groups, preferably one to three, more preferably one to two hydroxy groups. Suitable examples include but are not limited to —OCH₂OH, —OCH₂CH₂OH, —OCH(OH)—CH₃, —OCH(OH)CH₂OH, —OCH₂CH₂—CH₂OH, —OC(CH₂OH)₃, and the like.

As used herein, unless otherwise noted, the term “fluorinated C₁₋₄alkoxy” shall mean any C₁₋₄alkoxy group as defined above substituted with one or more fluoro groups, preferably one to three fluoro group. Suitably examples include, but are not limited to —OCH₂F, —OCHF₂, —OCF₃, —OCH₂—CF₃, —OCF₂—CH₃, —OCH₂—CH₂—CH₂F, —OCH₂—CH₂—CF₃, —OC(CH₃)₂CF₃, —OC(CF₃)₃, and the like.

As used herein, unless otherwise noted, the term “hydroxy substituted fluorinated C₁₋₄alkoxy” shall mean any C₁₋₄alkoxy group as defined above, wherein the C₁₋₄alkoxy group is substituted with one or more, preferably one to three, preferably one, hydroxy group(s) and further substituted with one or more, preferably one to six, preferably one to three, fluoro group(s). Suitably examples include but are not limited to —OCF₂(OH), —OCH(OH)—CF₃, —OCH₂—CH(OH)—CF₃, —OCH₂—CH₂—CH(OH)—CF₃, and the like.

As used herein, unless otherwise noted, the term “aryl” shall mean any unsubstituted carbocylic aromatic groups, preferably phenyl or naphthyl.

As used herein, unless otherwise noted, the term “heterocyclyl” shall denote any monocyclic, saturated, partially unsaturated, or aromatic ring structure containing at least one heteroatom selected from the group consisting of O, N and S, optionally containing one to three additional heteroatoms independently selected from the group consisting of O, N and S; or any saturated, partially unsaturated, partially aromatic or aromatic bicyclic, benzo-fused, bridged or spiro-cyclic ring system containing at least one heteroatom selected from the group consisting of O, N and S, optionally containing one to four additional heteroatoms independently selected from the group consisting of O, N and S. Preferably, the heterocyclyl is any three to twelve membered, preferably five to ten membered, more preferably five, six, nine or ten membered (for example, five to six membered or nine to ten membered) ring structure as herein defined. Suitable examples include, but are not limited to, azetidinyl, oxetanyl, thientanyl, pyrrolyl, furyl, thienyl, oxazolyl, imidazolyl, purazolyl, isoxazolyl, isothiazolyl, triazolyl, thiadiazolyl, pyridyl, pyridazinyl, pyrimidinyl, pyrazinyl, pyranyl, furazanyl, indolizinyl, indolyl, isoindolinyl, indazolyl, benzofuryl, benzothienyl, benzimidazolyl, benzthiazolyl, benzoisothiazolyl, purinyl, quinolizinyl, quinolinyl, isoquinolinyl, isothiazolyl, cinnolinyl, phthalazinyl, quinazolinyl, quinoxalinyl, naphthyridinyl, pteridinyl, pyrrolinyl, pyrrolidinyl, dioxalanyl, imidazolinyl, imidazolidinyl, pyrazolinyl, pyrazolidinyl, piperidinyl, dioxanyl, morpholinyl, dithianyl, thiomorpholinyl, piperazinyl, trithianyl, indolinyl, chromenyl, 2,3-dihydrobenzofuryl, 2,3-dihydrobenzo[b][1,4]dioxinyl, benzo[d][1,3]dioxolyl, 3,4-dihydro-1,7-naphthyridinyl, imidazo[1,2-a]pyridinyl, pyrazolo[1,5-a]pyridinyl, and the like.

As used herein, unless otherwise noted, the term “5 to 6 membered heterocyclyl” shall denote any five to six membered monocyclic, saturated, partially unsaturated or aromatic heterocyclyl group as described above. Unless otherwise noted, the 5 to 6 membered heterocyclyl may be attached at any heteroatom or carbon atom of the ring such that the result is a stable structure. Suitable examples include, but are not limited to azetidinyl, oxetanyl, tetrahydrofuryl, furyl, thiophenyl, pyrrolyl, pyrrolidinyl, oxazolyl, thiazolyl, isoxazolyl, pyrazolyl, imidazolyl, triazolyl, isothiazolyl, dioxolanyl, pyrazolidinyl, thiadiazolyl, pyranyl, tetrahydropyranyl, pyridinyl, dioxanyl, morpholinyl, dithianyl, thiomorpholinyl, pyridazinyl, pyrimidinyl, pyrazinyl, piperidinyl, piperazinyl, triazinyl, oxazinyl, isoxazinyl, oxathiazinyl, and the like.

As used herein, unless otherwise noted, the term “9 to 10 membered heterocyclyl” shall mean any nine to ten membered saturated, partially unsaturated, partially aromatic or aromatic, bicyclic, benzo-fused, bridged or spiro-cyclic heterocyclyl group as described above. Unless otherwise noted, the 9 to 10 membered heterocyclyl may be attached at any heteroatom or carbon atom of the ring such that the result is a stable structure. Suitable examples include, but are not limited to indolenyl, indolyl, isoindolyl, indolizinyl, indolinyl, benzofuryl, benzothiophenyl, indazolyl, benzimidazolyl, benzthiazolyl, purinyl, quinolinyl, isoquinolinyl, quinolizinyl, quinazolinyl, cinnolinyl, phthalazinyl, quinoxalinyl, naphthyridinyl, pteridinyl, quinuclidinyl, thionaphthenyl, isobenzazolyl, pyrano[3,4-b]pyrrolyl, anthranyl, benzopyranyl, chromenyl, coumarinyl, benzopyronyl, 3,4-dihydro-1,7-naphthyridinyl, imidazo[1,2-a]pyridinyl, pyrazolo[1,5-a]pyridinyl, and the like.

As used herein, unless otherwise noted, the term “heteroaryl” shall denote any five or six membered, monocyclic, aromatic ring structure containing at least one heteroatom selected from the group consisting of O, N and S, optionally containing one to three additional heteroatoms independently selected from the group consisting of O, N and S; or nine or ten membered, bicyclic, aromatic ring structure containing at least one heteroatom selected from the group consisting of O, N and S, optionally containing one to four additional heteroatoms independently selected from the group consisting of O, N and S. The heteroaryl may be bound through any ring atom which results in a stable structure. Suitable examples include, but are not limited to, furanyl, thienyl, furazanyl, oxazolyl, imidazolyl, pyrrolyl, pyrazolyl, thiazolyl, isoxazolyl, isothiazolyl, triazolyl, tetrazolyl, thiadiazolyl, oxadiazolyl, pyrazyl, pyridyl, pyridazinyl, pyrimidinyl, pyrazinyl, triazinyl, indolyl, indolizinyl, isoindolinyl, indazolyl, benzofuranyl, benzothienyl, benzimidazolyl, benzthiazolyl, benzoisothiazolyl, purinyl, quinolizinyl, quinolinyl, isoquinolinyl, isothiazolyl, cinnolinyl, phthalazinyl, quinazolinyl, quinoxalinyl, naphthyridinyl, pteridinyl, imidazo[1,2-a]pyridin-7-yl, [1,2,4]triazolo[4,3-a]pyridin-7-yl, imidazo[1,2-a]pyridinyl, pyrazolo[1,5-a]pyridinyl, and the like.

As used herein, unless otherwise noted, the term “five to six membered heteroaryl” shall denote any five or six membered, monocyclic, aromatic ring structure containing at least one heteroatom selected from the group consisting of O, N and S, optionally containing one to three additional heteroatoms independently selected from the group consisting of O, N and S. The heteroaryl may be bound through any ring atom which results in a stable structure. Suitable examples include, but are not limited to, furyl, thienyl, furazanyl, oxazolyl, imidazolyl, pyrrolyl, pyrazolyl, thiazolyl, isoxazolyl, isothiazolyl, triazolyl, tetrazolyl, thiadiazolyl, oxadiazolyl, pyrazyl, pyridyl, pyridazinyl, pyrimidinyl, pyrazinyl, triazinyl, and the like.

When a particular group is “substituted” (e.g. C_{X-Y}alkyl, heteroaryl, etc.), that group may have one or more substituents, preferably from one to five substituents, more preferably from one to three substituents, most preferably from one to two substituents, independently selected from the list of substituents.

With reference to substituents, the term “independently” means that when more than one of such substituents is possible, such substituents may be the same or different from each other.

Under standard nomenclature used throughout this disclosure, the terminal portion of the designated side chain is described first, followed by the adjacent functionality toward the point of attachment. Thus, for example, a “phenyl-(C₁-C₆alkylene)-amino-carbonyl-(C₁-C₆alkylene)-” substituent refers to a group of the formula

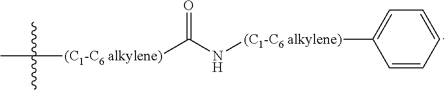

Abbreviations used in the specification, particularly the Schemes and Examples, are as listed in the Table A, below:

TABLE A
Abbreviations

| | |
|---|---|
| Ac = | Acetyl (i.e. —C(O)CH₃) |
| AcOH or | Acetic Acid |
| HOAc = | |
| ACN or | Acetonitrile |
| MeCN = | |
| aq. = | Aqueous |
| Boc or BOC = | tert-Butoxyloxycarbonyl (i.e. |
| | —C(O)—O—C(CH₃)₃) |
| Boc₂O = | Di-tert-butyl decarbonate |
| BSA = | Bovine Serum Albumin |
| CHAPS = | 3-[(3-Cholamidopropyl)dimethylammonio]- |
| | 1-propanesulfonate |
| CuPC = | Copper(II) phthalocyanine |
| Dave-Phos | 2-Dicyclohexylphosphino-2′-(N,N- |
| Ligand = | dimethylamino)biphenyl |
| dba = | Dibenzylideneacetone |
| DCM = | Dichloromethane |
| DCE = | 1,2-Dichloroethane |
| DEA = | Diethanolamine |
| DIEA or | Diisopropylethyl Amine |
| DIPEA = | |
| DMA = | Dimethylacetamide |
| DME (biological | Diabetic Macular Edema |
| context) = | |
| DME (Chemistry | Dimethoxy ethane |
| context) = | |
| DMF = | N,N-Dimethylformamide |
| DMP or | 3-Oxo-1λ⁵,2-benziodoxole-1,1,1(3H)- |
| Dess Martin | triyl |
| Periodinane = | triacetate |
| DMSO = | Dimethylsulfoxide |
| dppf = | 1,1′-Bis(diphenylphosphino)ferrocene |
| EA or EtOAc = | Ethyl Acetate |
| EDCI = | 1-Ethyl-3-(3-dimethylaminopropyl) carbodiimide |
| ee = | Enantiomeric Excess |
| ER = | End Point Read (assay) |
| equiv. = | Equivalents |
| ES or ESI = | Electrospray ionization |
| Et = | Ethyl |
| EtOH = | Ethanol |
| Et₂O = | Diethyl Ether |
| FA = | Formic Acid |
| FXIa = | Factor XIa |
| Grubbs' 2^{nd} | (1,3-Bis(2,4,6-trimethylphenyl)-2- |
| Catalyst = | imidazolidinylidene)dichloro(phenylmethylene) |
| | (tricyclohexylphosphine)ruthenium |
| HAE = | Hereditary Angioedema |
| HATU = | (1-[Bis(dimethylamino)methylene]-1H-1,2,3- |
| | triazolo[4,5-b]pyridinium 3-oxide |
| | hexafluorophosphate |
| Hex = | Hexanes |
| HPLC = | High Performance Liquid Chromatography |
| KHMDS | Potassium bis(trimethylsilyl)amide |
| KIN = | Kinetic Read (assay) |
| KOAc = | Potassium Acetate |
| LC-MS or | Liquid chromatography-mass spectrometry |
| LC/MS = | |
| LiHMDS = | Lithium bis(trimethylsilyl)amide |
| Me = | Methyl |
| MeOH = | Methanol |
| 2-MeTHF = | 2-Methyl-tetrahydrofuran |
| MOM = | Methoxy methyl |
| Ms or mesyl = | Methylsulfonyl (i.e. —SO₂—CH₃) |
| MTBE or | Methyl tert-Butyl Ether |
| MtBE = | |
| NaOAc = | Sodium Acetate |
| NaOt-Bu = | Sodium tert-Butoxide |
| NBS = | N-Bromosuccinimide |
| n-BuCH(Et)CO₂K = | Potassium ethylhexanoate |
| n-BuLi = | n-Butyl Lithium |
| NCS = | N-Chlorosuccinimide |
| NH₄OAc = | Ammonium Acetate |
| NIS = | N-Iodosuccinimide |
| NMR = | Nuclear Magnetic Resonance |
| Oms or | Methanesulfonate (i.e. —O—SO₂—CH₃) |
| mesylate = | |
| Otf or | Trifluoromethanesulfonyl (i.e. —O—SO═—CF₃) |
| triflate = | |
| Ots or | p-Toluenesulfonate (i.e. —O—SO₂-(p- |
| tosylate = | methylphenyl)) |
| Pd/C = | Palladium on Carbon (catalyst) |
| Pd(Oac)₂ = | Palladium (II) Acetate |
| Pd(dppf)Cl₂ or | [1,1′-Bis(diphenylphosphino)ferrocene] |
| PdCl₂(dppf) = | Palladium (II) Dichloride |
| PdCl₂(PPh₃)₂ or | Bis(triphenylphosphine)palladium (II) Dichloride |
| Pd(PPh₃)₂Cl_{2 =} | |
| Pd₂(dba)₃ or | Tris(dibenzylideneacetone)dipalladium(0) |
| Pd₂dba₃ = | |
| Pd(PPh₃)₄ = | Tetrakis(triphenylphosphine)palladium(0) |
| PE = | Petroleum ether |
| PK = | Plasma Kallikrein |
| PPh₃ = | Triphenylphosphine |
| RFU = | Relative Fluorescence Unit |
| sat. = | Saturated |
| Selectfluor = | 1-Chloromethyl-4-fluoro-1,4- |
| | diazoniabicyclo[2.2.2]octane |
| | bis(tetrafluoroborate) |
| SFC | Supercritical Fluid Chromatography (purification) |
| (purification) = | |
| TBAF = | Tetra-n-butylammonium fluoride |
| TBSOTf = | tert-Butyldimethylsilyl triflate |
| t-BuOK or | Potassium tert-Butoxide |
| KOt-Bu = | |
| TEA or | Triethylamine |
| Et₃N = | |
| Tf or | Trifluoromethylsulfonyl (i.e. —SO₂—CF₃) |
| triflyl = | |
| TFA = | Trifluoroacetic acid |
| THF = | Tetrahydrofuran |
| THP = | Tetrahydropyranyl |
| TMS = | Trimethysilyl |
| TMSCHN₂ = | Trimethylsilyldiazomethane |
| TMSN₃ = | Trimethylsilyl azide |
| Tris (buffer) = | 2-Amino-2-(hydroxymethyl)-1,3-propanediol |
| Ts or tosyl = | —SO₂-(p-methylphenyl) |

As used herein, the symbol or notation shall denote the presence of a stereogenic center.

Where the compounds according to the present invention have at least one chiral center, they may accordingly exist as enantiomers. Where the compounds possess two or more chiral centers, they may additionally exist as diastereomers or stereoisomers. It is to be understood that all such isomers and mixtures thereof are encompassed within the scope of the present invention. It is further understood that atropisomers (a specific type of stereoisomer resulting from steric or other hinderances to rotation) are also encompassed within the scope of the present invention.

Preferably, wherein the compound is present as an enantiomer, the enantiomer is present at an enantiomeric excess of greater than or equal to about 80%, more preferably, at an enantiomeric excess of greater than or equal to about 90%, more preferably still, at an enantiomeric excess of greater than or equal to about 95%, more preferably still, at an enantiomeric excess of greater than or equal to about 98%, most preferably, at an enantiomeric excess of greater than or equal to about 99%. Similarly, wherein the compound is present as a diastereomer or stereoisomer, the diastereomer or stereoisomer is present at a diastereomeric or stereoisomeric excess of greater than or equal to about 80%, more preferably, at a diastereomeric or stereoisomeric excess of greater than or equal to about 90%, more preferably still, at a diastereomeric or stereoisomeric excess of greater than or equal to about 95%, more preferably still, at a diastereomeric or stereoisomeric excess of greater than or equal to about 98%, most preferably, at a diastereomeric or stereoisomeric excess of greater than or equal to about 99%.

In some embodiments, the present invention is directed to compounds of formula (I), compounds of formula (II), compounds of formula (III) and/or compounds of formula (IV) in an enantiomeric excess of one of the R- or S-enantiomers (at the R³ stereocenter denoted with the “*”). In some embodiments of the present invention, the compound of formula (I) is present in an enantiomeric excess of one of the R- or S-enantiomers (at the R³ stereocenter denoted with the “*”) of about 55%, about 60%, about 65%, about 70%, about 75%, about 80%, about 85%, about 90%, about 91%, about 92%, about 93%, about 94%, about 95%, about 96%, about 97%, about 98% or about 99%. Preferably the compound of formula (I), compound of formula (II), compound of formula (III) and/or compound of formula (IV) is present in an enantiomeric excess of one of the R- or S-enantiomers (at the R³stereocenter denoted with the “*”) of greater than or equal to about 80%, preferably greater than or equal to about 90%, more preferably greater than or equal to about 93%, more preferably greater than or equal to about 95%, more preferably greater than or equal to about 97%, more preferably greater than or equal to about 98%, more preferably greater than or equal to about 99%.

In some embodiments, the present invention is directed to compounds of formula (I), compounds of formula (II), compounds of formula (III) and/or compounds of formula (IV) in a diastereomeric or stereoisomeric excess of one of the possible diastereomers or stereoisomers. In some embodiments of the present invention, the compound of formula (I), compound of formula (II), compound of formula (III) and/or compound of formula (IV) is present in a diastereomeric or stereoisomeric excess of one of the possible diastereomers or stereoisomers, of about 55%, about 60%, about 65%, about 70%, about 75%, about 80%, about 85%, about 90%, about 91%, about 92%, about 93%, about 94%, about 95%, about 96%, about 97%, about 98% or about 99%. Preferably, the compound of formula (I), compound of formula (II), compound of formula (III) and/or compound of formula (IV) is present in a diastereomeric or stereoisomeric excess of one of the possible diastereomers or stereoisomers of greater than or equal to about 80%, preferably greater than or equal to about 90%, more preferably greater than or equal to about 93%, more preferably greater than or equal to about 95%, more preferably greater than or equal to about 97%, more preferably greater than or equal to about 98%, more preferably greater than or equal to about 99%.

Furthermore, some of the crystalline forms for the compounds of the present invention may exist as polymorphs and as such are intended to be included in the present invention. In addition, some of the compounds of the present invention may form solvates with water (i.e., hydrates) or common organic solvents, and such solvates are also intended to be encompassed within the scope of this invention.

As used herein, unless otherwise noted, the term “isotopologues” shall mean molecules that differ only in their isotopic composition. More particularly, an isotopologue of a molecule differs from the parent molecule in that it contains at least one atom which is an isotope (i.e. has a different number of neutrons from its parent atom).

For example, isotopologues of water include, but are not limited to, “light water” (HOH or H₂O), “semi-heavy water” with the deuterium isotope in equal proportion to protium (HDO or ¹H²HO), “heavy water” with two deuterium isotopes of hydrogen per molecule (d₂O or ²H₂O), “super-heavy water” or tritiated water (T₂O or ³H₂O), where the hydrogen atoms are replaced with tritium (³H) isotopes, two heavy-oxygen water isotopologues (H₂¹⁸O and H₂¹⁷O) and isotopologues where the hydrogen and oxygen atoms may each independently be replaced by isotopes, for example the doubly labeled water isotopologue d₂¹⁸O.

It is intended that within the scope of the present invention, any one or more element(s), in particular when mentioned in relation to a compound of formula (I), shall comprise all isotopes and isotopic mixtures of said element(s), either naturally occurring or synthetically produced, either with natural abundance or in an isotopically enriched form. For example, a reference to hydrogen includes within its scope ¹H, ²H (D), and ³H (T). Similarly, references to carbon and oxygen include within their scope respectively ¹²C, ¹³C and ¹⁴C and ¹⁶O and ¹⁸O. The isotopes may be radioactive or non-radioactive. Radiolabelled compounds of formula (I), compounds of formula (II), compounds of formula (III) and/or compounds of formula (IV) may comprise one or more radioactive isotope(s) selected from the group of ³H, ¹¹C, ¹⁸F, ¹²²I, ¹²³I, ¹²⁵I, ¹³¹I, ⁷⁵Br, ⁷⁶Br, ⁷⁷Br and ³²Br. Preferably, the radioactive isotope is selected from the group of ³H, ¹¹C and ¹⁸F.

As used herein, unless otherwise noted, the term “isotopomers” shall mean isomers with isotopic atoms, having the same number of each isotope of each element but differing in their position. Isotopomers include both constitutional isomers and stereoisomers solely based on isotopic location. For example, CH₃CHDCH₃ and CH₃CH₂CH₂D are a pair of constitutional isotopomers of n-propane; whereas (R)—CH₃CHDOH and (S)—CH₃CHDOH or (Z)—CH₃CH═CHD and (E)-CH₃CH═CHD are examples of isotopic stereoisomers of ethanol and n-propene, respectively.

It is further intended that the present invention includes the compounds described herein, including all isomers thereof (including, but not limited to tautomers, stereoisomers, enantiomers, diastereomers, atropisomers, isotopologues, and the like).

As used herein, unless otherwise noted, the term “isolated form” shall mean that the compound is present in a form which is separate from any solid mixture with another compound(s), solvent system or biological environment. In an embodiment of the present invention, the compound of formula (I), compound of formula (II), compound of formula (III) or compound of formula (IV) is present in an isolated form.

As used herein, unless otherwise noted, the term “substantially pure form” shall mean that the mole percent of impurities in the isolated compound is less than about 5 mole percent, preferably less than about 2 mole percent, more preferably, less than about 0.5 mole percent, most preferably, less than about 0.1 mole percent. In an embodiment of the present invention, the compound of formula (I), compound of formula (II), compound of formula (III) or compound of formula (IV) is present as a substantially pure form.

As used herein, unless otherwise noted, the term “substantially free of a corresponding salt form(s)” when used to describe the compound of formula (I) (or compound of formula (II), compound of formula (IV), or compound of formula (IV)) shall mean that mole percent of the corresponding salt form(s) in the isolated base of formula (I) (or compound of formula (II), compound of formula (IV), or compound of formula (IV), respectively) is less than about 5 mole percent, preferably less than about 2 mole percent, more preferably, less than about 0.5 mole percent, most preferably less than about 0.1 mole percent. In an embodiment of the present invention, the compound of formula (I) is present in a form which is substantially free of corresponding salt form(s). In an embodiment of the present invention, the compound of formula (II) is present in a form which is substantially free of corresponding salt form(s). In an embodiment of the present invention, the compound of formula (III) is present in a form which is substantially free of corresponding salt form(s). In an embodiment of the present invention, the compound of formula (IV) is present in a form which is substantially free of corresponding salt form(s).

For use in medicine, the salts of the compounds of this invention refer to non-toxic “pharmaceutically acceptable salts”. Other salts may, however, be useful in the preparation of compounds according to this invention or of their pharmaceutically acceptable salts. Suitable pharmaceutically acceptable salts of the compounds include acid addition salts which may, for example, be formed by mixing a solution of the compound with a solution of a pharmaceutically acceptable acid such as hydrochloric acid, sulfuric acid, fumaric acid, maleic acid, succinic acid, acetic acid, benzoic acid, citric acid, tartaric acid, carbonic acid or phosphoric acid. Furthermore, where the compounds of the invention carry an acidic moiety, suitable pharmaceutically acceptable salts thereof may include alkali metal salts, e.g., sodium or potassium salts; alkaline earth metal salts, e.g., calcium or magnesium salts; and salts formed with suitable organic ligands, e.g., quaternary ammonium salts. Thus, representative pharmaceutically acceptable salts include, but are not limited to, the following: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, oleate, pamoate (embonate), palmitate, pantothenate, phosphate/diphosphate, polygalacturonate, salicylate, stearate, sulfate, subacetate, succinate, tannate, tartrate, teoclate, tosylate, triethiodide and valerate.

Representative acids which may be used in the preparation of pharmaceutically acceptable salts include, but are not limited to, the following: acids including acetic acid, 2,2-dichloroacetic acid, acylated amino acids, adipic acid, alginic acid, ascorbic acid, L-aspartic acid, benzenesulfonic acid, benzoic acid, 4-acetamidobenzoic acid, (+)-camphoric acid, camphorsulfonic acid, (+)-(1S)-camphor-10-sulfonic acid, capric acid, caproic acid, caprylic acid, cinnamic acid, citric acid, cyclamic acid, dodecylsulfuric acid, ethane-1,2-disulfonic acid, ethanesulfonic acid, 2-hydroxy-ethanesulfonic acid, formic acid, fumaric acid, galactaric acid, gentisic acid, glucoheptonic acid, D-gluconic acid, D-glucoronic acid, L-glutamic acid, α-oxo-glutaric acid, glycolic acid, hippuric acid, hydrobromic acid, hydrochloric acid, (+)-L-lactic acid, (±)-DL-lactic acid, lactobionic acid, maleic acid, (±)-L-malic acid, malonic acid, (±)-DL-mandelic acid, methanesulfonic acid, naphthalene-2-sulfonic acid, naphthalene-1,5-disulfonic acid, 1-hydroxy-2-naphthoic acid, nicotinic acid, nitric acid, oleic acid, orotic acid, oxalic acid, palmitic acid, pamoic acid, phosphoric acid, L-pyroglutamic acid, salicylic acid, 4-amino-salicylic acid, sebacic acid, stearic acid, succinic acid, sulfuric acid, tannic acid, (+)-L-tartaric acid, thiocyanic acid, p-toluenesulfonic acid and undecylenic acid.

Representative bases which may be used in the preparation of pharmaceutically acceptable salts include, but are not limited to, the following: bases including ammonia, L-arginine, benethamine, benzathine, calcium hydroxide, choline, deanol, diethanolamine, diethylamine, 2-(diethylamino)-ethanol, ethanolamine, ethylenediamine, N-methyl-glucamine, hydrabamine, 1H-imidazole, L-lysine, magnesium hydroxide, 4-(2-hydroxyethyl)-morpholine, piperazine, potassium hydroxide, 1-(2-hydroxyethyl)-pyrrolidine, secondary amine, sodium hydroxide, triethanolamine, tromethamine and zinc hydroxide.

The present invention includes within its scope prodrugs of the compounds of this invention. In general, such prodrugs will be functional derivatives of the compounds which are readily convertible in vivo into the required compound. Thus, in the methods of treatment of the present invention, the term “administering” shall encompass the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in “Design of Prodrugs”, ed. H. Bundgaard, Elsevier, 1985.

The term “subject” as used herein, refers to an animal, preferably a mammal, most preferably a human, who has been the object of treatment, observation or experiment. Preferably, the subject has experienced and/or exhibited at least one symptom of the disease or disorder to be treated and/or prevented.

The term “therapeutically effective amount” as used herein, means that amount of active compound or pharmaceutical agent that elicits the biological or medicinal response in a tissue system, animal or human that is being sought by a researcher, veterinarian, medical doctor or other clinician, which includes alleviation of the symptoms of the disease or disorder being treated.

As used herein, the term “composition” is intended to encompass a product comprising the specified ingredients in the specified amounts, as well as any product which results, directly or indirectly, from combinations of the specified ingredients in the specified amounts.

As used herein, unless otherwise noted, the terms “treating”, “treatment” and the like, shall include the management and care of a subject or patient, preferably a mammal, more preferably a human, for the purpose of combating a disease, condition, or disorder and includes the administration of a compound of the present invention to prevent the onset of the symptoms or complications, alleviate the symptoms or complications, slow the progression of the disease or disorder, or eliminate the disease, condition, or disorder. The terms “treating” or “treatment” further include: (a) inhibiting the disease-state, i.e., arresting its development; and/or (b) relieving the disease-state, i.e., causing regression of the disease state.

As used herein, “prevention” covers the preventive treatment of a subclinical disease-state in a mammal, particularly in a human, aimed at reducing the probability of the occurrence of a clinical disease-state. Patients are selected for preventative therapy based on factors that are known to increase risk of suffering a clinical disease state compared to the general population.

As used herein, “prophylaxis” is the protective treatment of a disease state to reduce and/or minimize the risk and/or reduction in the risk of recurrence of a disease state by administering to a patient a therapeutically effective amount of at least one of the compounds of the present invention or a tautomer, stereoisomer, isotopologue, a pharmaceutically acceptable salt, thereof. Patients may be selected for prophylaxis therapy based on factors that are known to increase risk of suffering a clinical disease state compared to the general population. For prophylaxis treatment, conditions of the clinical disease state may or may not be presented yet. “Prophylaxis” treatment can be divided into (a) primary prophylaxis and (b) secondary prophylaxis. Primary prophylaxis is defined as treatment to reduce or minimize the risk of a disease state in a patient that has not yet presented with a clinical disease state, whereas secondary prophylaxis is defined as minimizing or reducing the risk of a recurrence or second occurrence of the same or similar clinical disease state.

As used herein, “risk reduction” covers therapies that lower the incidence of development of a clinical disease state. As such, primary and secondary prevention therapies are examples of risk reduction.

As used herein, the terms “combination” and “pharmaceutical combination” refer to either: 1) a fixed dose combination in one dosage unit form, or 2) a non-fixed dose combination, optionally packaged together for combined administration.

### Examples

The following Examples are set forth to aid in the understanding of the invention and are not intended and should not be construed to limit in any way the invention set forth in the claims which follow thereafter.

Where an Example which follow hereinafter lists only analytical measurements such as LC/MS, ¹H NMR, ¹⁹F NMR, etc. (rather than reaction step details), it will be understood that the title compound was prepared according to the procedures as described in the synthesis schemes and Examples herein, selecting and substituting suitable reagents and reactants, as would be readily recognized by those skilled in the art.

Unless otherwise indicated in the examples, all temperature is expressed in Centigrade (° C.). All reactions were conducted under an inert atmosphere at ambient temperature unless otherwise noted. Unless otherwise specified, reaction solutions were stirred at room temperature under a N_{2(g)} or Ar_{(g)} atmosphere. Reagents employed without synthetic details are commercially available or made according to known methods, for example according to literature procedures. When solutions were “concentrated to dryness”, they were concentrated using a rotary evaporator under reduced pressure, when solutions were dried, they were typically dried over a drying agent such as MgSO₄ or Na₂SO₄. Where a synthesis product is listed as having been isolated as a residue, it will be understood by those skilled in the art that the term “residue” does not limit the physical state in which the product was isolated and may include, for example, a solid, an oil, a foam, a gum, a syrup, and the like.

In obtaining the compounds described in the examples below and the corresponding analytical data, the following experimental and analytical protocols were followed unless otherwise indicated.

LC-MS: Unless otherwise indicated, the analytical LC-MS system used consisted of a Shimadzu LCMS-2020 with electrospray ionization (ESI) in positive ion detection mode with 20ADXR pump, SIL-20ACXR autosampler, CTO-20AC column oven, M20A PDA Detector and LCMS 2020 MS detector. The column was a HALO a C18 30*5.0 mm, 2.7 μm. The mobile phase A was water containing 0.05% TFA and mobile phase B was acetonitrile containing 0.05% TFA. The gradient was from 5% mobile phase B to 100% (95%) in 2.0 min, hold 0.7 min, then revert to 5% mobile phase B over 0.05 min and maintain for 0.25 min. The Column Oven (CTO-20AC) was operated at a 40.0° C. The flow rate was 1.5 mL/min, and the injection volume was 1 μl. PDA (SPD-M20A) detection was in the range 190-400 nm. The MS detector, which was configured with electrospray ionization as ionizable source; Acquisition mode: Scan; Nebulizing Gas Flow: 1.5 L/min; Drying Gas Flow: 15 L/min; Detector Voltage: Tuning Voltage ±0.2 kv; DL Temperature: 250° C.; Heat Block Temperature: 250° C.; Scan Range: 90.00-900.00 m/z. ELSD (Alltech 3300) detector Parameters: Drift Tube Temperature: 60±5° C.; N₂ Flow-Rate: 1.8±0.2 L/min. Mobile phase gradients were optimized for the individual compounds. Calculated mass corresponds to the exact mass.

Preparative HPLC: Unless otherwise noted, preparative HPLC purifications were performed with Waters Auto purification system (2545-2767) with a 2489 UV detector. The column was selected from one of the following: Waters C18, 19×150 mm, 5 μm; XBridge Prep OBD C18 Column, 30×150 mm 5 μm; XSelect CSH Prep C18 OBD Column, 5 μm, 19*150 mm; XBridge Shield RP18 OBD Column, 30×150 mm, 5 μm; Xselect CSH Fluoro Phenyl, 30×150 mm, 5 μm; or YMC-Actus Triart C18, 30×150 mm, 5 μm. The mobile phases consisted of mixtures of acetonitrile (5-95%) in water containing 0.1% FA or 10 mmol/L NH₄HCO₃. Flow rates were maintained at 25 mL/min, the injection volume was 1200 μL, and the UV detector used two channels 254 nm and 220 nm. Mobile phase gradients were optimized for the individual compounds.

Chiral chromatography: Chiral analytical chromatography was performed on one of ChiralpakAS, AD, Chiralcel OD, OJ Chiralpak IA, IB, IC, ID, IE, IF, IG, IH columns (Daicel Chemical Industries, Ltd.) (R,R)-Whelk-O1, (S,S)-Whelk-O1 columns (Regis technologies, Inc.) CHIRAL Cellulose-SB, SC, SA columns (YMC Co., Ltd.) as noted, at different column size (50×4.6 mm, 100×4.6 mm, 150×4.6 mm, 250×4.6 mm, 50×3.0 mm, 100×3.0 mm), with percentage of either ethanol in hexane (% Et/Hex) or isopropanol in hexane (% IPA/Hex) as isocratic solvent systems, or using supercritical fluid (SFC) conditions.

Normal phase flash chromatography: Unless otherwise noted, normal phase flash column chromatography (FCC) was performed on silica gel with pre-packaged silica gel columns (such as RediSep®), using ethyl acetate (EtOAc)/hexanes, ethyl acetate (EtOAc)/Petroleum ether (b.p. 60-90° C.), CH₂Cl₂/MeOH, or CH₂Cl₂/10% 2N NH₃ in MeOH, as eluent.

¹H NMR: Unless otherwise noted, ¹H NMR spectra were acquired using 400 MHz spectrometers (or 300 MHz spectrometers) in DMSO-d₆ solutions. The nuclear magnetic resonance (NMR) spectral characteristics refer to chemical shifts (6) are expressed in parts per million (ppm). Tetramethylsilane (TMS) was used as internal reference in DMSO-d₆ solutions, and residual CH₃OH peak or TMS was used as internal reference in CD₃OD solutions. Coupling constants (J) are reported in hertz (Hz). The nature of the shifts as to multiplicity is reported as s (singlet), d (doublet), t (triplet), q (quartet), dd (double doublet), dt (double triplet), m (multiplet), br (broad).

### Synthesis Examples: Intermediate Compounds

### Intermediate Example A: 2-Bromo-1-(2-(1-((tert-butyldimethylsilyl)oxy)ethyl)-3-fluoropyridin-4-yl)ethan-1-one

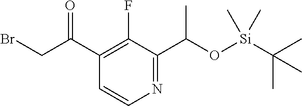 [SMILES: CC(O[Si](C)(C)C(C)(C)C)c1nccc(C(=O)CBr)c1F]

A mixture of 2-(1-((tert-butyldimethylsilyl)oxy)ethyl)-4-(1-ethoxyvinyl)-3-fluoropyridine (490 mg, 1.50 mmol, 1 equiv.) and NBS (214 mg, 1.20 mmol, 0.8 equiv.) in THF/H₂O (V:V=3:1, 8 mL) was stirred 15 min at room temperature. The mixture was diluted with H₂O and extracted with ethyl acetate twice. The combined organic layers was washed with brine, dried over Na₂SO₄, and concentrated to yield 2-bromo-1-(2-(1-((tert-butyldimethylsilyl)oxy)ethyl)-3-fluoropyridin-4-yl)ethan-1-one as a yellow oil.

### Intermediate Example B: 2-Bromo-1-(2-(((tert-butyldimethylsilyl)oxy)methyl)-3-fluoropyridin-4-yl)ethan-1-one

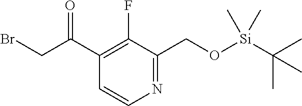 [SMILES: CC(C)(C)[Si](C)(C)OCc1nccc(C(=O)CBr)c1F]

### Step 1: 2-(((Tert-butyldimethylsilyl)oxy)methyl)-4-(1-ethoxyvinyl)-3-fluoropyridine

To a mixture of 2-(((tert-butyldimethylsilyl)oxy)methyl)-3-fluoro-4-iodopyridine (2.0 g, 5.45 mmol, 1.0 equiv.) in 1,4-dioxane (20 mL) was added tributyl(1-ethoxyvinyl)stannane (2.2 mL, 6.54 mmol, 1.2 equiv.) and Pd(PPh₃)₂Cl₂ (0.38 g, 0.55 mmol, 0.1 equiv.) under N₂. The solution was stirred at 90° C. overnight under nitrogen. The reaction was added water and extracted with ethyl acetate twice. The combined organic layers were washed with brine, dried over Na₂SO₄ and concentrated under vacuum and purified by Al₂O₃ gel chromatography (0-10% EA/PE) to yield 2-(((tert-butyldimethylsilyl)oxy)methyl)-4-(1-ethoxyvinyl)-3-fluoropyridine as yellow oil. LC/MS: mass calculated for C₁₆H₂₆FNO₂Si: 311.17, measured: 312.20 [M+H]⁺.

### Step 2: 2-Bromo-1-(2-(((tert-butyldimethylsilyl)oxy)methyl)-3-fluoropyridin-4-yl)ethan-1-one

To a mixture of 2-(((tert-butyldimethylsilyl)oxy)methyl)-4-(1-ethoxyvinyl)-3-fluoropyridine (300 mg, 0.96 mmol, 1.0 equiv.) in THE (3 mL) and water (1 mL) was added N-bromosuccinimide (137 mg, 0.77 mmol, 0.8 equiv.). The reaction mixture was stirred at room temperature for 20 min. The reaction was added water and extracted with ethyl acetate twice. The combined organic layers were washed with brine, dried over Na₂SO₄, concentrated under vacuum to yield 2-bromo-1-(2-(((tert-butyldimethylsilyl)oxy)methyl)-3-fluoropyridin-4-yl)ethan-1-one as a yellow oil. LC/MS: mass calculated, for C₁₄H₂₁BrFNO₂Si: 361.05, measured: 362.00 [M+H]⁺

### Intermediate Example C: 5-(2-bromoacetyl)-1-methyl-1H-pyrazole-4-carbonitrile

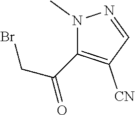

### Step 1: 1-(4-Bromo-1-methyl-1H-pyrazol-5-yl)ethan-1-ol

To a solution of 4-bromo-1-methyl-1H-pyrazole-5-carbaldehyde (10 mL) was added methylmagnesium bromide (7.9 mL) at −78° C. The resulting mixture was stirred for 2 h at 0° C. Saturated NH₄Cl was added, the mixture was extracted with EA. The combined extracts were washed with water, saturated brine and dried over anhydrous Na₂SO₄. The organic phase was concentrated to yield 1-(4-bromo-1-methyl-1H-pyrazol-3-yl)ethan-1-ol as colorless oil, which was used in the next step without further purification. LC/MS: mass calculated for C₆H₉BrN₂O: 203.99, measured: 205.05 [M+H]⁺.

### Step 2: 1-(4-Bromo-1-methyl-1H-pyrazol-5-yl)ethan-1-one

To a solution of 1-(4-bromo-1-methyl-1H-pyrazol-5-yl)ethan-1-ol (500 mg, 2.43 mmol, 1 eq.) in dichloromethane (20 mL) was added Dess-Martin periodinane (2.1 g, 4.877 mmol). The mixture stirred at room temperature for 2 h. The residue was purified by silica gel chromatography with EA/PE (0-70%) to yield 1-(4-bromo-1-methyl-1H-pyrazol-5-yl)ethan-1-one as colorless oil. LC/MS: mass calculated for C₆H₇BrN₂O: 201.97, measured: 202.90 [M+H]⁺.

### Step 3: 5-Acetyl-1-methyl-1H-pyrazole-4-carbonitrile

To a solution of 1-(4-bromo-1-methyl-1H-pyrazol-5-yl)ethan-1-one (400 mg, 1.970 mmol, 1 eq.), dicyanozinc (231 mg, 1.97 mol, 1 eq), dppf (109 mg, 0.19 mmol, 0.1 eq.) in DMA (10 mL) under an nitrogen atmosphere, was added Pd₂(dba)₃ (180 mg, 0.19 mmol, 1 eq.). The resulting solution was stirred at 100° C. for 2 h. The reaction was quenched with water (20 mL). The resulting mixture was extracted with ethyl acetate (3×50 mL). The organic layers were combined, dried over anhydrous sodium sulfate, filtered and concentrated. The residue was purified by silica gel chromatography (0→20% ethyl acetate/petroleum ether) to yield the 5-acetyl-1-methyl-1H-pyrazole-4-carbonitrile as a light yellow solid.

### Step 4: 5-(2-Bromoacetyl)-1-methyl-1H-pyrazole-4-carbonitrile

To a solution of 5-acetyl-1-methyl-1H-pyrazole-4-carbonitrile (200 mg, 1.34 mmol, 1 eq.) in glacial acetic acid (10 mL) was added hydrogen bromide (986 mg, 4.02 mmol, 3 eq.) and pyridinium tribromide (429 mg, 1.34 mmol, 1 eq.). The reaction mixture was stirred 2 h at room temperature. The reaction mixture was concentrated under vacuum to yield 5-(2-bromoacetyl)-1-methyl-1H-pyrazole-4-carbonitrile as a brown solid, which was used in the next step(s) without further purification.

### Intermediate Example D: 1-(2-amino-5-fluoropyridin-4-yl)-2-bromoethan-1-one

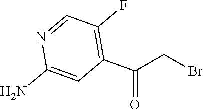 [SMILES: Nc1cc(C(=O)CBr)c(F)cn1]

### Step 1: 4-(1-Ethoxyvinyl)-5-fluoropyridin-2-amine

To a solution of 5-fluoro-4-iodopyridin-2-amine (1.5 g, 6.30 mmol, 1 equiv) and tributyl(1-ethoxyvinyl)stannane (3.4 g, 9.5 mmol, 1.5 equiv) in 1,4-dioxane (15 mL) was added Pd(PPh₃)₄ (364 mg, 0.3 mmol, 0.1 equiv.) under N₂. The reaction mixture was stirred overnight at 80° C., then cooled to room temperature and quenched with water, extracted with EA, washed with brine, dried over Na₂SO₄ and concentrated under vacuum. The residue was purified by silica gel chromatography with EA/PE (0→66%) to yield 4-(1-Ethoxyvinyl)-5-fluoropyridin-2-amine as yellow oil. LC/MS: mass calculated for C₉H₁₁FN₂O: 182.09, measured: 183.2 [M+H]⁺.

### Step 2: 1-(2-Amino-5-fluoropyridin-4-yl)-2-bromoethan-1-one

To a solution of 5-fluoro-4-((prop-1-en-2-yloxy)methyl)pyridin-2-amine (0.5 g, 2.74 mmol, 1.0 equiv) was added 1-bromopyrrolidine-2,5-dione (488 mg, 2.74 mmol, 1 equiv) in THE (8 mL) and water (4 mL). The reaction mixture was stirred 1 h at room temperature, then quenched with water, extracted with EA, washed with brine, dried over Na₂SO₄ and concentrated under vacuum. The residue was purified by silica gel chromatography with EA/PE (0→66%) to yield 1-(2-amino-5-fluoropyridin-4-yl)-2-bromoethan-1-one as red solid.

### Intermediated E: 1-(2-amino-4-(trifluoromethyl)thiazol-5-yl)-2-bromoethan-1-one

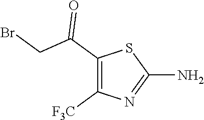 [SMILES: Nc1nc(C(F)(F)F)c(C(=O)CBr)s1]

To a solution of 1-(2-amino-4-(trifluoromethyl)thiazol-5-yl)ethan-1-one (700 mg, 3.33 mmol, 1 eq) in acetic acid (30 mL) were added hydrogen bromide (1.6 g, 6.66 mmol, 2 eq) and pyridinium tribromide (1.1 g, 3.33 mmol, 1 eq). The reaction mixture was stirred 2 h at room temperature. The reaction mixture was concentrated under vacuum. The pH value of the aqueous phase was adjusted to 10 with saturation sodium bicarbonate solution, and the resulting mixture was extracted with ethyl acetate. The combined organic layer was dried over anhydrous sodium sulfate and concentrated under vacuum to yield 1-(2-amino-4-(trifluoromethyl)thiazol-5-yl)-2-bromoethan-1-one as an yellow solid which was used directly in the next step. LC/MS: mass calculated for C₆H₄BrF₃N₂OS: 287.92, measured: 288.90 [M+H]⁺.

### Intermediate F: 2-Bromo-1-(1-methyl-1H-1,2,3-triazol-5-yl)ethan-1-one

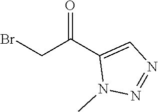

### Step 1: 1-(1-Methyl-1H-1,2,3-triazol-5-yl)ethan-1-one

To a solution of 1-methyl-1H-1,2,3-triazole (7.2 g, 86.20 mmol, 1.0 equiv.) in THE (80 mL) was added dropwise n-BuLi (37.9 mL, 94.82 mmol, 1.05 equiv.) under N₂ at −78° C. The reaction mixture was stirred at −78° C. for 1 h, then a solution of N-methoxy-N-methylacetamide (16.0 g, 155.16 mmol, 1.8 equiv.) in THE (10 mL) was added dropwise. The reaction mixture was stirred 2 h at −78° C. and then warmed to room temperature. The reaction was quenched with NH₄Cl_{(aq.)} The resulting mixture was extracted with ethyl acetate twice. The combined organic layers were washed with brine, dried over Na₂SO₄ and concentrated under vacuum. The residue was purified by silica gel chromatography with EA/PE (0→66%) to yield 1-(1-methyl-1H-1,2,3-triazol-5-yl)ethan-1-one as yellow oil. LC/MS: mass calculated for C₅H₇N₃O: 125.06, measured (ES, m/z): 126.00 [M+H]⁺.

### Step 2: 2-Bromo-1-(1-methyl-1H-1,2,3-triazol-5-yl)ethan-1-one

To a solution of 1-(4-bromopyridin-2-yl)ethan-1-one (8.0 g, 63.93 mmol, 1.0 equiv.) in HBr/AcOH (800 mL) was added pyridinium tribromide (20.1 g, 62.65 mmol, 1.0 equiv.). The reaction mixture was stirred at room temperature overnight. The mixture was extracted with ethyl acetate twice. The combined organic layers were washed with NaHCO₃, dried and concentrated in vacuum to yield 2-bromo-1-(1-methyl-1H-1,2,3-triazol-5-yl)ethan-1-one as a black solid. LC/MS: mass calculated for C₅H₆BrN₃O: 202.97, measured (ES, m/z): 204.05 [M+H]⁺.

### Intermediate Example G: 4-(2-bromoacetyl)-3-methylpicolinonitrile

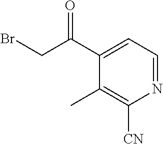

### Step 1: 4-(1-Ethoxyvinyl)-3-methylpicolinonitrile

To a solution of 4-chloro-3-methylpicolinonitrile (1 g, 6.55 mmol, 1 equiv.) and tributyl(1-ethoxyvinyl)stannane (2.4 mL, 7.20 mmol, 1.1 equiv.) in 1,4-dioxane (20 mL) was added tetrakis(triphenylphosphine)palladium (379 mg, 0.32 mmol, 0.05 equiv.). The reaction mixture was stirred 8 h at 100° C. under an atmosphere of nitrogen. After the mixture was cooled down, the mixture was quenched with water, and extracted with ethyl acetate. The combined organic layer was dried over anhydrous sodium sulfate and concentrated under vacuum. The residue was applied onto an aluminum oxide gel column (0-40% ethyl acetate/petroleum ether) to yield 4-(1-ethoxyvinyl)-3-methylpicolinonitrile as light color oil. LC/MS: mass calculated for C₁₁H₁₂N₂O: 188.09, measured: 189.10 [M+H]⁺.

### Step 2: 4-(2-Bromoacetyl)-3-methylpicolinonitrile

To a solution of 4-(1-ethoxyvinyl)picolinonitrile (1.2 g, 6.37 mmol, 1 eq.) in THF/H₂O (15 mL/10 mL) was NBS (1.1 g, 6.37 mmol, 1 eq.). After the reaction mixture was stirred at room temperature for 2 h, water was added. The mixture was extracted with ethyl acetate. The solvent was removed under vacuum and the residue was purified by silica gel chromatography with EA/PE (0→70%) to yield 4-(2-bromoacetyl)-3-methylpicolinonitrile as an off-white solid. LC/MS: mass calculated for C₉H₇BrN₂O: 237.97, measured: 238.90 [M+H]⁺.

### Intermediate Example H: 2-bromo-1-(imidazo[1,2-a]pyridin-6-yl)ethan-1-one

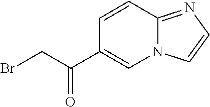 [SMILES: O=C(CBr)c1ccc2nccn2c1]

To a solution of 1-(imidazo[1,2-a]pyridin-6-yl)ethanone (2.0 g, 12.48 mmol, 1.00 equiv.) in acetic acid (40 mL) was added HBr (6.1 g, 33% in acetic acid, 24.97 mmol, 2.00 equiv.) and Br₂ (2.0 g, 12.48 mmol, 1.00 equiv.). The resulting mixture was stirred at room temperature. for 2 h. The reaction mixture was concentrated to yield 2-bromo-1-(imidazo[1,2-a]pyridin-6-yl)ethanone as an off-white solid.

### Intermediate Example I: 1-(6-amino-2-methylpyridin-3-yl)-2-bromoethan-1-one

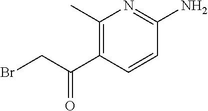

### Step 1: 5-(1-Ethoxyvinyl)-6-methylpyridin-2-amine

To a solution of 5-bromo-6-methylpyridin-2-amine (4 g, 21.38 mmol, 1 eq.) and tributyl(1-ethoxyvinyl)stannane (8.5 g, 23.52 mmol, 1.1 eq.) in 1,4-dioxane (20 mL) was added tetrakis(triphenylphosphine)palladium (1.2 g, 1.06 mmol, 0.05 eq.). The reaction mixture was stirred overnight at 100° C. under an atmosphere of nitrogen. The mixture was quenched with water, and extracted with ethyl acetate. The combined organic layer was dried over anhydrous sodium sulfate and concentrated under vacuum to yield 5-(1-ethoxyvinyl)-6-methylpyridin-2-amine as yellow oil, which was used in the next step without further purification. LC/MS: mass calculated for C₁₀H₁₄N₂O: 178.11, measured: 179.10 [M+H]⁺.

### Step 2: 1-(6-Amino-2-methylpyridin-3-yl)ethan-1-one

To a solution of 5-(1-ethoxyvinyl)-6-methylpyridin-2-amine (3.8 g, 21.32 mmol, 1 Eq.) in tetrahydrofuran (20 mL) was added hydrochloric acid (2 M in water, 20 mL). The reaction mixture was stirred at room temperature for 2 h. The reaction mixture was washed with EA. The aqueous phase was adjusted to pH 10 with NaHCO₃ solution (1 M) and extracted with ethyl acetate. The combined organic layer was dried over anhydrous sodium sulfate and concentrated under vacuum. The residue was purified by reverse column chromatography with CH₃CN/0.05% TFA water (5%-50%) to yield 1-(6-amino-2-methylpyridin-3-yl)ethan-1-one as an light yellow solid.

### Step 3: 1-(6-Amino-2-methylpyridin-3-yl)-2-bromoethan-1-one

To a solution of 1-(6-amino-2-methylpyridin-3-yl)ethan-1-one (750 mg, 4.99 mmol, 1 eq) in acetic acid (40 mL) were added hydrogen bromide (2.4 g, 9.98 mmol, 2 eq) and pyridinium tribromide (958 mg, 2.99 mmol, 0.6 Eq). The reaction mixture was stirred 2 h at room temperature. The reaction mixture was concentrated under vacuum. The aqueous phase was adjusted to pH 10 with saturated sodium bicarbonate solution and extracted with ethyl acetate. The combined organic layer was dried over anhydrous sodium sulfate and concentrated under vacuum. The residue was applied onto a silica gel column (0-40% ethyl acetate/petroleum ether) to yield 1-(6-amino-2-methylpyridin-3-yl)-2-bromoethan-1-one as a light yellow solid. LC/MS: mass calculated for C₈H₉BrN₂O: 227.99, measured: 228.95 [M+H]⁺.

### Intermediate Example J: 2-bromo-1-(pyrazolo[1,5-a]pyridin-5-yl)ethan-1-one

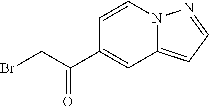 [SMILES: O=C(CBr)c1ccn2nccc2c1]

### Step 1: 5-(1-Ethoxyvinyl)pyrazolo[1,5-a]pyridine

5-Bromopyrazolo[1,5-a]pyridine (300 mg, 1.52 mmol), tributyl(1-ethoxyvinyl)stannane (66 mg, 1.82 mmol) and tetrakis(triphenylphosphine)palladium (176 mg, 0.15 mmol) were added to a 25 mL flask under N₂, followed by addition of 1, 4-dioxane (5 mL). The reaction mixture was heated at reflux with stirring for 3 h. The reaction mixture was diluted by ethyl acetate (50 mL) and washed with water (20 mL) three times and the organic layer was dried by anhydrous sodium sulfate. The solvent was removed under vacuum to yield 5-(1-ethoxyvinyl)pyrazolo[1,5-a]pyridine as a light yellow oil, which was used in next step without further purification.

### Step 2: 1-(Pyrazolo[1,5-a]pyridin-5-yl)ethan-1-one

To the solution of 5-(1-ethoxyvinyl)pyrazolo[1,5-a]pyridine (199 mg, 1.05 mmol) in tetrahydrofuran (10 mL) was added 2 N hydrogen chloride (1.0 mL). The reaction mixture was stirred for 2 h at room temperature, then diluted with ethyl acetate (30 mL), washed by water (10 mL) three times, and the organic layer was dried over anhydrous sodium sulfate. The solvent was removed and the residue was purified by silica gel column chromatography (0-60% EA/PE) to yield 1-(pyrazolo[1,5-a]pyridin-5-yl)ethan-1-one as a light yellow oil.

### Step 3: 2-Bromo-1-(pyrazolo[1,5-a]pyridin-5-yl)ethan-1-one

A mixture of 1-(pyrazolo[1,5-a]pyridin-5-yl)ethan-1-one (240 mg, 1.5 mmol), hydrobromic acid, acetic acid solution 33% (1.28 g) and glacial acetic acid (5 mL) was stirred at 0° C. To the resulting orange solution was added pyridinium tribromide (432 mg, 1.35 mmol), the reaction mixture was stirred at room temperature for 2 h, then diluted with ethyl acetate (40 mL) and washed with water (15 mL), dried by Na₂SO₄ and concentrated to yield a residue, which was purified by chromatography (80 g, PE/EA, 0>>>60%) to yield 2-bromo-1-(pyrazolo[1,5-a]pyridin-5-yl)ethan-1-one as a yellow solid.

### Intermediate Example K: 2-Bromo-1-(2-(2-((tert-butyldimethylsilyl)oxy)-2-methylpropoxy)-3-fluoropyridin-4-yl)ethan-1-one

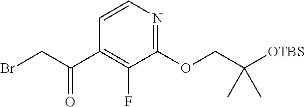 [SMILES: CC(C)(COc1nccc(C(=O)CBr)c1F)O[Si](C)(C)C(C)(C)C]

### Step 1: 1-((3-Fluoro-4-iodopyridin-2-yl)oxy)-2-methylpropan-2-ol

To a solution of 2-methylpropane-1,2-diol (0.8 g, 9.1 mmol, 1.1 equiv.) in DMF (20 mL) was added NaH (0.2 mg, 9.1 mmol, 1.1 equiv.). The resulting mixture was stirred at 0° C. for 0.5 h. The reaction was added 2,3-difluoro-4-iodopyridine (2 g, 8.3 mmol, 1.1 equiv.). The resulting mixture was stirred at 25° C. for 1 h. The mixture was extracted with ethyl acetate and NH₄Cl. The organic layers were combined, dried over anhydrous sodium sulfate, filtered and concentrated to yield 1-((3-fluoro-4-iodopyridin-2-yl)oxy)-2-methylpropan-2-ol as a yellow oil.

### Step 2: 2-(2-((Tert-butyldimethylsilyl)oxy)-2-methylpropoxy)-3-fluoro-4-iodopyridine

To a solution of 1-((3-fluoro-4-iodopyridin-2-yl)oxy)-2-methylpropan-2-ol (2.3 g, 7.4 mmol, 1.0 equiv.) in DCM (30 mL) was added TBSOTf (2.5 g, 9.6 mmol, 1.3 equiv.) and 2,6-Lutidine (2.4 g, 22.2 mmol, 3.0 equiv.). The resulting mixture was stirred at 25° C. for 5 hrs. The mixture was extracted with DCM (100 mL). The organic layers were combined, dried over anhydrous sodium sulfate, filtered, and concentrated under vacuum. The residue was purified by silica gel chromatography (0→20% EA/PE) to yield 2-(2-((tert-butyldimethylsilyl)oxy)-2-methylpropoxy)-3-fluoro-4-iodopyridine as a yellow oil.

### Step 3: 2-(2-((Tert-butyldimethylsilyl)oxy)-2-methylpropoxy)-4-(1-ethoxyvinyl)-3-fluoropyridine

To a solution of 2-(2-((tert-butyldimethylsilyl)oxy)-2-methylpropoxy)-3-fluoro-4-iodopyridine (1.9 g, 4.5 mmol, 1.0 equiv.) in 1,4-dioxane (15 mL) were added tributyl(1-ethoxyvinyl)stannane (3.2 g, 8.9 mmol, 2.0 equiv.) and Pd(PPh₃)₄ (0.5 g, 0.4 mmol, 0.1 equiv.). The resulting mixture was stirred at 90° C. for 6 hrs. The mixture was diluted with water and extracted with ethyl acetate (150 mL). The organic layers were combined, dried over anhydrous sodium sulfate, filtered and concentrated. The residue was purified by Al₂O₃ chromatography (0→10% ethyl acetate/petroleum ether) to yield 2-(2-((tert-butyldimethylsilyl)oxy)-2-methylpropoxy)-4-(1-ethoxyvinyl)-3-fluoropyridineas a yellow oil.

### Step 4: 2-Bromo-1-(2-(2-((tert-butyldimethylsilyl)oxy)-2-methylpropoxy)-3-fluoropyridin-4-yl)ethan-1-one

To a solution of 2-(2-((tert-butyldimethylsilyl)oxy)-2-methylpropoxy)-4-(1-ethoxyvinyl)-3-fluoropyridine (1 g, 2.7 mmol, 1.0 equiv.) in THE (10 mL) and H₂O (2.5 mL) was added NBS (0.4 g, 2.4 mmol, 0.9 equiv.). The resulting mixture was stirred at 25° C. for 0.5 h, diluted with water and extracted with ethyl acetate (100 mL). The organic layers were combined, dried over anhydrous sodium sulfate, filtered and concentrated to yield 2-bromo-1-(2-(2-((tert-butyldimethylsilyl)oxy)-2-methylpropoxy)-3-fluoropyridin-4-yl)ethan-1-one as a yellow solid.

### Intermediate Example L: Methyl-d3 (4-(2-chloroacetyl)phenyl)carbamate

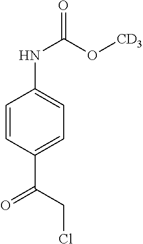 [SMILES: [2H]C([2H])([2H])OC(=O)Nc1ccc(C(=O)CCl)cc1]

To a solution of aluminum chloride (5.2 g, 38.91 mmol, 3.0 equiv.) in dichloroethane (15 mL) was added 2-chloroacetyl chloride (2.2 g, 19.46 mmol, 1.5 equiv.), stirring the mixture at room temperature for 0.5 h, then added (²H₃)methyl N-phenylcarbamate (2.0 g, 12.97 mmol 1.0 equiv.) at 0° C. The resulting mixture was maintained under nitrogen and stirred at 80° C. for 2 h, After cooling to room temperature, the reaction was quenched with ice water (100 ml). The reaction mixture was extracted with DCM (3×100 ml). The organic layers were combined, dried over anhydrous sodium sulfate, filtered and concentrated. The residue was purified by silica gel chromatography (0-40% ethyl acetate/petroleum ether) to yield (²H₃)methyl N-[4-(2-chloroacetyl)phenyl]carbamate as yellow solid. LC/MS: mass calculated for C₁₀H₇CID₃NO₃: 230.05 measured: 231.1 [M+H]⁺.

### Intermediate Example M: 2-Bromo-1-(3-fluoro-2-(3,3,3-trifluoro-2-hydroxypropoxy)pyridin-4-yl)ethan-1-one

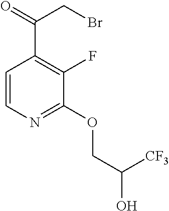 [SMILES: O=C(CBr)c1ccnc(OCC(O)C(F)(F)F)c1F]

To a solution of 3-((4-(1-ethoxyvinyl)-3-fluoropyridin-2-yl)oxy)-1,1,1-trifluoropropan-2-ol (900 mg, 3.04 mmol, 1.0 equiv) in THE (10 mL) with H₂O (5 mL) was added NBS (543 mg, 3.04 mmol, 1.0 equiv). The reaction mixture was stirred overnight at room temperature, then quenched with water and extracted with ethyl acetate. The combined organic layer was washed with brine, dried over Na₂SO₄ and concentrated under vacuum. The residue was purified by silica gel chromatography (0→70% EA/PE) to yield 2-bromo-1-(3-fluoro-2-(3,3,3-trifluoro-2-hydroxypropoxy)pyridin-4-yl)ethan-1-one as a yellow solid.

### Intermediate Example N: 2-bromo-1-(2-(1-((tert-butyldimethylsilyl)oxy)cyclopropyl)-3-fluoropyridin-4-yl)ethan-1-one

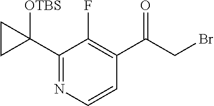 [SMILES: CC(C)(C)[Si](C)(C)OC1(c2nccc(C(=O)CBr)c2F)CC1]

### Step 1: 2-(1-((Tert-butyldimethylsilyl)oxy)vinyl)-3-fluoropyridine

To a solution of 1-(3-fluoropyridin-2-yl)ethan-1-one (6.0 g, 43.12 mmol, 1.0 equiv.) and triethylamine (13.1 g, 129.37 mmol, 3.0 equiv.) in DCM (60 mL) was added TBSOTf (14.8 g, 56.06 mmol, 1.3 equiv.) at 0° C. The mixture was stirred at room temperature for 1 h and quenched with water (20 mL). The resulting mixture was extracted with DCM (3×50 mL) and combined, dried over anhydrous sodium sulfate, filtered and concentrated. The residue was purified by silica gel chromatography (0→20% ethyl acetate/petroleum ether) to yield the 2-(1-((tert-butyldimethylsilyl)oxy)vinyl)-3-fluoropyridine as a yellow oil.

### Step 2: 2-(1-((Tert-butyldimethylsilyl)oxy)cyclopropyl)-3-fluoropyridine

To a solution of diethylzinc (52.6 mL, 78.93 mmol, 1.5 M in toluene, 4.0 equiv.) in DCM (80 mL) was added chloroiodomethane (20.9 g, 118.39 mmol, 6.0 equiv.) under nitrogen at 0° C. After 0.5 h of stirring, 2-(1-((tert-butyldimethylsilyl)oxy)vinyl)-3-fluoropyridine (5.0 g, 19.73 mmol, 1.0 equiv.) in DCM (10 mL) was added at 0° C. The resulting mixture was maintained under nitrogen and stirred at room temperature for 1 h and quenched with saturated ammonium chloride solution (20 mL). The resulting mixture was extracted with ethyl acetate (3×20 mL) and the organic layers were combined, dried over anhydrous sodium sulfate, filtered and concentrated under reduced pressure. The residue was purified by silica gel chromatography (0→20% ethyl acetate/petroleum ether) to yield the 2-(1-((tert-butyldimethylsilyl)oxy)cyclopropyl)-3-fluoropyridine as a yellow oil.

### Step 3: 2-(1-((Tert-butyldimethylsilyl)oxy)cyclopropyl)-3-fluoro-4-iodopyridine

To a solution of 2-(1-((tert-butyldimethylsilyl)oxy)cyclopropyl)-3-fluoropyridine (1.4 g, 5.24 mmol, 1.0 equiv.) in THE (20 mL) was added lithium diisopropylamide (3.4 mL, 6.81 mmol, 2.0 M in THE/Hexane, 1.1 equiv.) at −78° C. After 0.5 h, the solution of 12 (1.5 g, 5.76 mmol, 1.3 equiv.) in THE (4 mL) was added at −78° C. The resulting mixture was maintained under nitrogen and stirred at −78° C. for 1 h, quenched with saturated ammonium chloride solution (20 mL) and extracted with ethyl acetate (3×20 mL). The organic layers were combined, dried over anhydrous sodium sulfate, filtered, and concentrated under reduced pressure. The residue was purified by silica gel chromatography (0→30% ethyl acetate/petroleum ether) to yield the 2-(1-((tert-butyldimethylsilyl)oxy)cyclopropyl)-3-fluoro-4-iodopyridine as a yellow oil.

### Step 4: 2-(1-((Tert-butyldimethylsilyl)oxy)cyclopropyl)-4-(1-ethoxyvinyl)-3-fluoropyridine

To a solution of 2-(1-((tert-butyldimethylsilyl)oxy)cyclopropyl)-3-fluoro-4-iodopyridine (1.0 g, 2.54 mmol, 1.0 equiv.) in 1,4-dioxane (10 mL) was added tributyl(1-ethoxyvinyl)stannane (1.8 g, 5.09 mmol, 2.0 equiv.) and Pd(PPh₃)₄ (294 mg, 0.25 mmol, 0.1 equiv.). The resulting mixture was stirred at 100° C. for 4 h under nitrogen. After cooling to room temperature, the resulting mixture was extracted with ethyl acetate (3×20 mL). The organic layers were combined, dried over anhydrous sodium sulfate, filtered and concentrated. The residue obtained was purified by silica gel chromatography (0→50% ethyl acetate/petroleum ether) to yield the 2-(1-((tert-butyldimethylsilyl)oxy)cyclopropyl)-4-(1-ethoxyvinyl)-3-fluoropyridine as a yellow oil.

### Step 5: 2-Bromo-1-(2-(1-((tert-butyldimethylsilyl)oxy)cyclopropyl)-3-fluoropyridin-4-yl)ethan-1-one

To a solution of 2-(1-((tert-butyldimethylsilyl)oxy)cyclopropyl)-4-(1-ethoxyvinyl)-3-fluoropyridine (1.0 g, 2.96 mmol, 1.0 equiv.) in THE (8 mL) was added H₂O (2 mL) was added NBS (422 mg, 2.37 mmol, 0.8 equiv.) at 0° C. The reaction was stirred at room temperature for 1 h. The resulting mixture was extracted with ethyl acetate (2×20 mL). The organic layers were combined, dried over anhydrous sodium sulfate, filtered and concentrated to yield the 2-bromo-1-(2-(1-((tert-butyldimethylsilyl)oxy)cyclopropyl)-3-fluoropyridin-4-yl)ethan-1-one as a yellow oil.

### Intermediate Example 0: 1-(3-aminobenzo[d]isothiazol-6-yl)-2-bromoethan-1-one

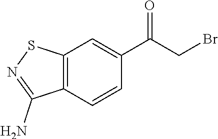 [SMILES: Nc1nsc2cc(C(=O)CBr)ccc12]

### Step 1: 6-Bromobenzo[d]isothiazol-3-amine

To a solution of 4-bromo-2-fluorobenzonitrile (3 g, 15.00 mmol, 1 eq) in DMSO (50 mL) was added sodium sulfide nonahydrate (2.2 g, 22.50 mmol, 1.5 eq) under nitrogen. The reaction mixture was stirred at 70° C. for 16 h, then quenched with water, extracted with ethyl acetate, washed with brine, dried over Na₂SO₄ and concentrated under vacuum. The residue was purified by silica gel chromatography with EA/PE (0-70%) to yield 1 g of 6-bromobenzo[d]isothiazol-3-amine as a white solid. LC/MS: mass calculated for C₇H₅BrN₂S: 227.94, measured: 231.00 [M+2+H]⁺.

### Step 2: 6-(1-Ethoxyvinyl)benzo[d]isothiazol-3-amine

To a solution of 4-bromo-2-fluorobenzonitrile (310 mg, 1.35 mmol, 1 eq) in 1,4-dioxane (5 mL) were added tributyl(1-ethoxyvinyl)tin (538 mg, 1.49 mmol, 1.1 eq), and tetrakis(triphenylphosphine)palladium (168 mg, 0.14 mmol, 0.05 eq). The resulting mixture was maintained under nitrogen and stirred at 100° C. for 3 h, then cooled to room temperature and quenched with water. The reaction mixture was extracted with ethyl acetate. The organic layers were combined, washed with brine, dried and concentrated under vacuum. The residue was purified by silica gel chromatography (0-20% ethyl acetate/petroleum ether) to yield 6-(1-ethoxyvinyl)benzo[d]isothiazol-3-amine as a white solid. LC/MS: mass calculated for C₁₁H₁₂N₂OS: 220.07, measured: 221.10 [M+H]⁺.

### Step 3: 1-(3-Aminobenzo[d]isothiazol-6-yl)ethan-1-one

To a solution of 6-(1-ethoxyvinyl)benzo[d]isothiazol-3-amine (200 mg, 0.91 mmol, 1 eq) in tetrahydrofuran (15 mL) was added 3 M HCl (2.0 mL, 6.00 mmol, 6.60 eq). The reaction mixture was stirred at room temperature for 0.5 h, then quenched with water. The reaction mixture was extracted with THF. The organic layers were combined, washed with brine, dried and concentrated under vacuum. The residue was purified by silica gel chromatography with (0-20% ethyl acetate/petroleum ether) to yield 1-(3-aminobenzo[d]isothiazol-6-yl)ethan-1-one as a white solid. LC/MS: mass calculated for C₉H₆N₂OS: 192.04, measured: 193.05 [M+H]⁺.

### Step 4: 1-(3-Aminobenzo[d]isothiazol-6-yl)-2-bromoethan-1-one

To a solution of 1-(3-aminobenzo[d]isothiazol-6-yl)ethan-1-one (63 mg, 0.33 mmol, 1 eq) in glacial acetic acid (2 mL) was added 33% hydrobromic acid solution (in acetic acid) (133 mg, 0.66 mmol, 2 eq) and pyridinium tribromide (103 mg, 0.32 mmol, 0.98 eq). The reaction mixture was stirred at room temperature for 2 h, concentrated under vacuum, then quenched with water, extracted with ethyl acetate, washed with brine, dried over Na₂SO₄ and concentrated under vacuum to yield of 1-(3-aminobenzo[d]isothiazol-6-yl)-2-bromoethan-1-one as a brown solid. LC/MS: mass calculated for C₉H₇BrN₂OS: 269.95, measured: 272.90 [M+2+H]⁺.

### Intermediate Example P: 1-(5-amino-3-(trifluoromethyl)pyrazin-2-yl)-2-bromoethan-1-one

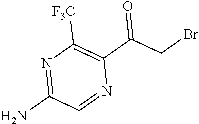 [SMILES: Nc1cnc(C(=O)CBr)c(C(F)(F)F)n1]

### Step 1: 5-Bromo-6-(trifluoromethyl)pyrazin-2-amine

To a solution of 6-(trifluoromethyl)pyrazin-2-amine (1.0 g, 6.13 mmol, 1 equiv) in DMF (20 mL) was added NBS (1.1 g, 6.13 mmol, 1.0 equiv) stepwise at 0° C. The mixture was stirred for 2 h at room temperature, then quenched with water, extracted with ethyl acetate, washed with brine, dried over Na₂SO₄ and concentrated under vacuum. The residue was purified by silica gel chromatography with EA/PE (0-40%) to yield 5-bromo-6-(trifluoromethyl)pyrazin-2-amine as an off-white solid. LC/MS: mass calculated for C₅H₃BrF₃N₃: 240.95, measured: 243.90 [M+2+H]⁺.

### Step 2: 5-(1-Ethoxyvinyl)-6-(trifluoromethyl)pyrazin-2-amine

To a solution of 5-bromo-6-(trifluoromethyl)pyrazin-2-amine (950 mg, 3.93 mmol, 1 equiv) in 1,4-dioxane (20 mL) was added 1-ethoxyvinyl-tri-n-butyltin (1.6 g, 4.32 mmol, 1.1 equiv) and Pd(PPh₃)₄ (227 mg, 0.20 mmol, 0.05 equiv) under N₂. The reaction mixture was stirred overnight at 100° C., then cooled to room temperature and quenched with water, extracted with ethyl acetate, dried over Na₂SO₄ and concentrated under vacuum. The residue was purified by silica gel chromatography with EA/PE (0-50%) to yield 5-(1-ethoxyvinyl)-6-(trifluoromethyl)pyrazin-2-amine as a light yellow solid. LC/MS: mass calculated for C₉H₁₀F₃N₃O: 233.08, measured: 234.10 [M+H]⁺.

### Step 3: 1-(5-Amino-3-(trifluoromethyl)pyrazin-2-yl)ethan-1-one

To a solution of 5-(1-ethoxyvinyl)-6-(trifluoromethyl)pyrazin-2-amine (0.5 g, 2.14 mmol, 1 equiv) in THE (10 mL) was added 2 M HCl (4 mL). The mixture was stirred for 1 h at room temperature. The pH was adjusted to 8-10 with NaHCO₃(aq.). The mixture was extracted with ethyl acetate, dried over Na₂SO₄ and concentrated under vacuum. The residue was purified by silica gel chromatography with EA/PE (0-50%) to yield 1-(5-amino-3-(trifluoromethyl)pyrazin-2-yl)ethan-1-one as a light yellow solid. LC/MS: mass calculated for C₇H₆F₃N₃O: 205.05, measured: 206.00 [M+H]⁺.

### Step 4: 1-(5-Amino-3-(trifluoromethyl)pyrazin-2-yl)-2-bromoethan-1-one

To a solution of 1-(5-amino-3-(trifluoromethyl)pyrazin-2-yl)ethan-1-one (430 mg, 2.10 mmol, 1 equiv) in acetic acid (10 mL) was added hydrogen bromide solution in acetic acid (1.0 g, 4.19 mmol, 2 equiv) followed by the addition of pyridinium tribromide (670 mg, 2.10 mmol, 1.0 equiv) slowly. The reaction mixture was stirred for 2 h at room temperature. The solids were collected by filtration and washed with diethyl ether, then evaporated under vacuum to yield 1-(5-amino-3-(trifluoromethyl)pyrazin-2-yl)-2-bromoethan-1-one hydrobromide as a grey solid. LC/MS: mass calculated for C₇H₅BrF₃N₃O: 282.96, measured: 285.90 [M+2+H]⁺.

### Intermediate Example Q: 1-(2-amino-3-fluoropyridin-4-yl)-2-bromoethan-1-one

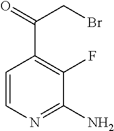 [SMILES: Nc1nccc(C(=O)CBr)c1F]

### Step 1: 3-fluoro-4-iodopyridin-2-amine

To a solution of 2,3-difluoro-4-iodopyridine (4 g, 16.5 mmol, 1.0 equiv.) in DMSO (50 mL) and H₂O (10 mL) was added acetamidine hydrochloride (1.8 g, 19.9 mmol, 1.2 equiv.) and NaOH (1.6 g, 41.4 mmol, 2.5 equiv.). The resulting mixture was stirred at 130° C. for 2 hrs. The mixture was filtered and concentrated under vacuum. The residue obtained was purified by silica gel chromatography (20-30% ethyl acetate/petroleum ether) to yield 3-fluoro-4-iodopyridin-2-amine as a white solid as a white solid.

### Step 2: 4-(1-ethoxyvinyl)-3-fluoropyridin-2-amine

To a solution of 3-fluoro-4-iodopyridin-2-amine (900 mg, 3.7 mmol, 1.0 equiv.) in 1,2-dioxane (15 mL) was added tributyl(1-ethoxyvinyl)tin (2.7 g, 7.5 mmol, 2.0 equiv.) and bis(triphenylphosphine)palladium(II) chloride (437 mg, 0.3 mmol, 0.1 equiv.). The resulting mixture was stirred at 100° C. for 16 hrs. The mixture was extracted with ethyl acetate (100 mL), the organic layer was combined, dried over anhydrous sodium sulfate, filtered and concentrated to yield 4-(1-ethoxyvinyl)-3-fluoropyridin-2-amine as a brown oil.

### Step 3: 1-(2-amino-3-fluoropyridin-4-yl)ethan-1-one

To a solution of 4-(1-ethoxyvinyl)-3-fluoropyridin-2-amine (600 mg, 3.2 mmol) in THE (15 mL) was added HCl (2 mL, 4 mmol, 2 M). The resulting mixture was stirred at 25° C. for 5 hrs. The pH value of the aqueous phase was adjusted to 7 with NaHCO₃ solution. The resulting mixture was extracted with ethyl acetate (150 mL). The organic layers were combined, dried over anhydrous sodium sulfate, filtered and concentrated. The residue obtained was purified by silica gel chromatography (30-45% ethyl acetate/petroleum ether) to yield 1-(2-amino-3-fluoropyridin-4-yl)ethan-1-one as a yellow solid as a yellow solid.

### Step 4: 1-(2-amino-3-fluoropyridin-4-yl)-2-bromoethan-1-one

To a solution of 1-(2-amino-3-fluoropyridin-4-yl)ethan-1-one (150 mg, 0.9 mmol, 1.0 equiv.) in acetic acid (4 mL) were added pyridinium tribromide (280.1 mg, 0.8 mmol, 0.9 equiv.) and hydrogen bromide (477 mg, 1.9 mmol, 2.0 equiv.). The resulting mixture was stirred at 25° C. for 2 hrs. The reaction mixture was filtered and concentrated to yield 1-(2-amino-3-fluoropyridin-4-yl)-2-bromoethan-1-one as a yellow solid.

### Intermediate Example R: 1-(5-amino-3-fluoropyrazin-2-yl)-2-bromoethan-1-one

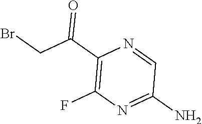 [SMILES: Nc1cnc(C(=O)CBr)c(F)n1]

### Step 1: 5-Bromo-6-fluoropyrazin-2-amine

To a solution of 6-fluoropyrazin-2-amine (770 mg, 6.81 mmol, 1.0 equiv) in DMF (8 mL) was added NBS (1.5 g, 8.17 mmol, 1.2 equiv) and stirred at room temperature for 3 h. The reaction mixture was concentrated and purified by flash column chromatography on silica gel (EA/PE, 1-40%) to yield 5-bromo-6-fluoropyrazin-2-amine as a yellow solid. LC/MS: mass calculated for C₄H₃BrFN₃: 190.95, measured: 192.00 [M+H]⁺.

### Step 2: 1-(5-Amino-3-fluoropyrazin-2-yl)ethan-1-one

To a solution of 5-bromo-6-fluoropyrazin-2-amine (1.1 g, 5.73 mmol, 1 equiv) and tributyl(1-ethoxyvinyl)stannane (2.3 ml, 6.88 mmol, 1.2 equiv) in 1,4-dioxane (15 mL) was added Pd(PPh₃)₂Cl₂ (402 mg, 0.57 mmol, 0.1 equiv) under N₂. The reaction mixture was stirred for 3 h at 90° C. The reaction mixture was added 3N HCl and stirred for 10 min and extracted with ethyl acetate, the organic layer was washed with brine and dried over anhydrous Na₂SO₄. The solvent was removed under reduced pressure and the residue was purified by flash column chromatography on silica gel (EA/PE, 1-40%) to yield 1-(5-amino-3-fluoropyrazin-2-yl)ethan-1-one as a yellow solid. LC/MS: mass calculated for C₆H₆FN₃O: 155.05, measured: 156.00 [M+H]⁺.

### Step 3: 1-(5-Amino-3-fluoropyrazin-2-yl)-2-bromoethan-1-one

A mixture of 1-(5-amino-3-fluoropyrazin-2-yl)ethan-1-one (150 mg, 0.97 mmol, 1.0 equiv), HBr/AcOH (0.3 mL) and Py.Br₃ (309 mg, 0.97 mmol, 1.0 equiv) in AcOH (3 mL) was stirred at room temperature for 2 h. The reaction mixture was concentrated, diluted with water, and extracted with ethyl acetate, the organic layer was washed with water and NaHCO₃ and dried over anhydrous Na₂SO₄. The solvent was removed under vacuum and the residue was purified by flash column chromatography on silica gel to yield 1-(5-amino-3-fluoropyrazin-2-yl)-2-bromoethan-1-one as a yellow solid. LC/MS: mass calculated for C₆H₅BrFN₃O: 232.96, measured: 234.00 [M+H]⁺.

### Intermediate Example S: N-(5-(2-bromoacetyl)-6-cyclopropylpyrazin-2-yl)acetamide

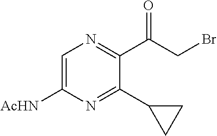 [SMILES: CC(=O)Nc1cnc(C(=O)CBr)c(C2CC2)n1]

### Step 1: N-(6-Cyclopropylpyrazin-2-yl)acetamide

To a solution of N-(6-bromopyrazin-2-yl)acetamide (0.9 g, 4.17 mmol, 1 equiv) in toluene (15 mL) and water (2 mL) was added cyclopropylboronic acid (0.7 g, 8.33 mmol, 2 equiv), potassium phosphate (2.7 g, 12.50 mmol, 3 equiv), Pd(OAc)₂ (94 mg, 0.42 mmol, 0.1 equiv) and tricyclohexylphosphonium tetrafluoroborate (307 mg, 0.83 mmol, 0.2 equiv) under N₂. The reaction mixture was stirred overnight at 100° C., then cooled to room temperature and quenched with water. The mixture was extracted with EA, washed with brine, dried over Na₂SO₄ and concentrated under vacuum. The residue was purified by silica gel chromatography with EA/PE (0-60%) to yield N-(6-cyclopropylpyrazin-2-yl)acetamide as a light yellow solid. LC/MS: mass calculated for C₉H₁₁N₃O: 177.09, measured: 178.30 [M+H]⁺.

### Step 2: N-(5-Bromo-6-cyclopropylpyrazin-2-yl)acetamide

To a solution of N-(6-cyclopropylpyrazin-2-yl)acetamide (540 mg, 3.05 mmol, 1 equiv) in DMF (10 mL) was added NBS (814 mg, 4.57 mmol, 1.5 equiv). The mixture was stirred for 5 h at room temperature, then quenched with water, extracted with EA, washed with brine, dried over Na₂SO₄ and concentrated under vacuum. The residue was purified by silica gel chromatography with EA/PE (0-60%) to yield N-(5-bromo-6-cyclopropylpyrazin-2-yl)acetamide as a light yellow solid. LC/MS: mass calculated for C₉H₁₀BrN₃O: 255.00, measured: 258.00 [M+2+H]⁺.

### Step 3: N-(6-Cyclopropyl-5-(1-ethoxyvinyl)pyrazin-2-yl)acetamide

To a solution of N-(5-bromo-6-cyclopropylpyrazin-2-yl)acetamide (240 mg, 0.937 mmol, 1 equiv) in 1,4-dioxane (10 mL) was added 1-ethoxyvinyl-tri-n-butyltin (372 mg, 1.03 mmol, 1.1 equiv) and Pd(PPh₃)₄ (108 mg, 0.09 mmol, 0.1 equiv) under N₂. The reaction mixture was stirred overnight at 100° C., then cooled to room temperature and quenched with water, extracted with EA, dried over Na₂SO₄ and concentrated under vacuum. The residue was purified by silica gel chromatography with EA/PE (0-50%) to yield N-(6-cyclopropyl-5-(1-ethoxyvinyl)pyrazin-2-yl)acetamide as light yellow oil. LC/MS: mass calculated for C₁₃H₁₇N₃O₂: 247.13, measured: 248.10 [M+H]⁺.

### Step 4: N-(5-(2-Bromoacetyl)-6-cyclopropylpyrazin-2-yl)acetamide

To a solution of N-(6-cyclopropyl-5-(1-ethoxyvinyl)pyrazin-2-yl)acetamide (130 mg, 0.53 mmol, 1 equiv) in THE (6 mL) and water (2 mL) was added NBS (112 mg, 0.63 mmol, 1.2 equiv). The mixture was stirred for 2 h at room temperature, then extracted with EA, washed with brine, dried over Na₂SO₄ and concentrated under vacuum. The residue was purified by silica gel chromatography with EA/PE (0-60%) to yield N-(5-(2-bromoacetyl)-6-cyclopropylpyrazin-2-yl)acetamide as a light yellow solid. LC/MS: mass calculated for C₁₁H₁₂BrN₃O₂: 297.01, measured: 299.90 [M+2+H]⁺.

### Intermediate Example T: 2-Bromo-1-(4-((4-methoxybenzyl)oxy)quinolin-7-yl)ethan-1-one

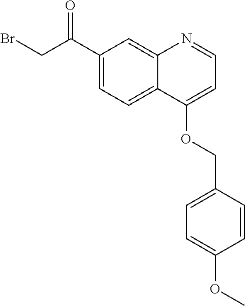 [SMILES: COc1ccc(COc2ccnc3cc(C(=O)CBr)ccc23)cc1]

### Step 1: 7-Bromo-4-((4-methoxybenzyl)oxy)quinoline

To a solution of 7-bromoquinolin-4-ol (1 g, 5.05 mmol, 1 equiv.) and (4-methoxyphenyl)methanol (0.76 g, 5.55 mmol, 1.1 equiv.) in THE (15 mL) was added triphenylphosphine (1.46 g, 5.55 mol, 1.1 equiv.), then a diisopropyl azodicarboxylate (1.22 g, 6.06 mmol, 1.2 equiv.) was added at 0° C. The reaction mixture was stirred 2 h at room temperature, then quenched with water, extracted with ethyl acetate, washed with brine, dried over Na₂SO₄ and concentrated under vacuum. The residue was purified by silica gel chromatography with EA/PE (0-100%) to yield (4-(((7-bromoquinolin-4-yl)oxy)methyl)phenyl)methanol as yellow oil. LC/MS: mass calculated for C₁₇H₁₄BrNO₂: 343.02, measured: 344.0 [M+H]⁺.

### Step 2: 1-(4-((4-Methoxybenzyl)oxy)quinolin-7-yl)ethan-1-one

To a solution of 7-bromo-4-((4-methoxybenzyl)oxy)quinoline (1 g, 5.05 mmol, 1 equiv.) and tributyl(1-ethoxyvinyl)stannane (2 g, 5.55 mol, 1.1 equiv.) in 1,4-dioxane (20 mL) was added Pd(PPh₃)₄ (0.292 g, 1.190 mmol, 0.2 equiv.) under N₂. The reaction mixture was stirred overnight at 80° C., then cooled to room temperature and quenched with water, extracted with ethyl acetate, washed with brine, dried over Na₂SO₄ and concentrated under vacuum. The residue was purified by silica gel chromatography with EA/PE (0-66%) to yield 7-(1-ethoxyvinyl)-4-((4-methoxybenzyl)oxy)quinoline as yellow oil. LC/MS: mass calculated for C₂₁H₂₁NO₃: 335.15, measured: 336.1 [M+H]⁺.

### Step 3: 2-Bromo-1-(4-((4-methoxybenzyl)oxy)quinolin-7-yl)ethan-1-one

To a solution of (4-(((7-(1-ethoxyvinyl)quinolin-4-yl)oxy)methyl)phenyl)methanol (0.3 g, 0.89 mmol, 1.0 equiv.) was added 1-bromopyrrolidine-2,5-dione (0.16 g, 0.89 mmol, 1 equiv.) in THE (6 mL) and water (3 mL). The reaction mixture was stirred 1 h at room temperature, then cooled to room temperature and quenched with water, extracted with ethyl acetate, washed with brine, dried over Na₂SO₄ and concentrated under vacuum. The residue was purified by silica gel chromatography with EA/PE (0-66%) to yield 2-bromo-1-(4-((4-methoxybenzyl)oxy)quinolin-7-yl)ethan-1-one as white solid. LC/MS: mass calculated for C₁₉H₁₆BrNO₃: 385.03, measured: 385.9 [M+H]⁺.

### Intermediate Example U: 2-Bromo-1-(2-methoxypyrimidin-4-yl)ethan-1-one

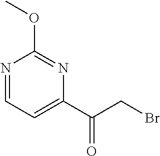

### Step 1: 1-(2-Methoxypyrimidin-4-yl)ethan-1-one

To a solution of 4-bromo-2-methoxypyrimidine (1.5 g, 7.93 mmol, 1 equiv) and tributyl(1-ethoxyvinyl)stannane (2.9 mL, 8.73 mmol, 1.1 equiv) in 1,4-dioxane (20 mL) was added tetrakis(triphenylphosphine)palladium (458 mg, 0.39 mmol, 0.05 equiv) under an atmosphere of nitrogen. The reaction mixture was stirred 8 h at 100° C. After the mixture was cooled, 1 M HCl (20 mL) was added and the reaction mixture was stirred for 20 min, quenched with water, and extracted with ethyl acetate. The combined organic layer was dried over anhydrous sodium sulfate and concentrated under vacuum. The residue was applied onto a silica gel column (0-40% ethyl acetate/petroleum ether) to yield 1-(2-methoxypyrimidin-4-yl)ethan-1-one as a light yellow solid.

### Step 2: 2-Bromo-1-(2-methoxypyrimidin-4-yl)ethan-1-one

To a solution of 1-(2-methoxypyrimidin-4-yl)ethan-1-one (600 mg, 3.94 mmol, 1 eq) in acetic acid (20 mL) were added hydrogen bromide (2.9 g, 11.83 mmol, 3 eq) and pyridinium tribromide (1.1 g, 3.54 mmol, 1 eq). The reaction mixture was stirred 2 h at room temperature. The mixture was concentrated, diluted with water (30 mL) and extracted with ethyl acetate (100 mL). The organic was washed with saturated NaHCO₃(50 mL). The organic layers were dried over anhydrous sodium sulfate, filtered and concentrated to yield 2-bromo-1-(2-methoxypyrimidin-4-yl)ethan-1-one as an light yellow solid, which was used in the next step without further purification.

### Intermediate Example V: 2-Bromo-1-(1,2,3-thiadiazol-5-yl)ethan-1-one

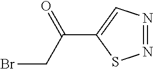

To a solution of 1-(1,2,3-thiadiazol-5-yl)ethan-1-one (400 mg, 3.12 mmol, 1.0 eq) in acetic acid (20 mL) were added hydrogen bromide (2.3 g, 9.36 mmol, 3.0 eq) and pyridinium tribromide (898 mg, 2.81 mmol, 1.0 eq). The reaction mixture was stirred 1 h at room temperature. The reaction mixture was filtered, and the solid was collected to yield 2-bromo-1-(1,2,3-thiadiazol-5-yl)ethan-1-one as a light yellow solid, which was used directly in the next step without further purification.

### Intermediate Example W: Methyl (5-(2-bromoacetyl)pyridin-2-yl)carbamate

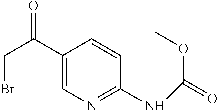

### Step 1: Methyl (5-acetylpyridin-2-yl)carbamate

To a solution of 1-(6-aminopyridin-3-yl)ethan-1-one (1 g, 7.34 mmol, 1 equiv) in MeOH (10 mL) was added di-tert-butyl dicarbonate (6.4 g, 29.37 mmol, 3 equiv) under N₂ at 0° C. The reaction mixture was stirred for 0.5 h at room temperature, then concentrated under vacuum to yield methyl (5-acetylpyridin-2-yl)carbamate. LC/MS: mass calculated for C₉H₁₀N₂O₃: 194.07, measured: 195.1 [M+H]⁺.

### Step 2: Methyl (5-(2-bromoacetyl)pyridin-2-yl)carbamate

To a solution of 1-(6-hydroxy-4-methylpyridin-3-yl)ethan-1-one (282 mg, 1.452 mmol, 1.0 equiv) in HBr/AcOH (7 mL), was added pyridinium tribromide (458 mg, 1.423 mmol, 0.980 equiv). The reaction mixture was stirred overnight at room temperature. The mixture was extracted with ethyl acetate. The combined organic layer was washed with NaHCO₃, dried and concentrated in vacuo to yield methyl (5-(2-bromoacetyl)pyridin-2-yl)carbamate as white solid. LC/MS: mass calculated for C₈H₉BrN₂O₃: 271.98, measured: 272.9 [M+H]⁺.

### Intermediate Example X: N-(5-(2-Bromoacetyl)pyridin-2-yl)cyclopropanecarboxamide

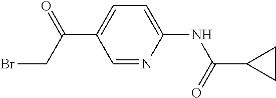 [SMILES: O=C(CBr)c1ccc(NC(=O)C2CC2)nc1]

### Step 1: N-(5-bromopyridin-2-yl)cyclopropanecarboxamide

To a solution of 5-bromopyridin-2-amine (2 g, 11.56 mmol) in DCM (15 mL) was added pyridine (1.8 g, 23.12 mmol) under N₂ at 0° C., then cyclopropanecarbonyl chloride (1.33 g, 12.71 mmol) was added with DCM (4 ml). The resulting mixture was maintained under nitrogen and stirred at 0° C. room temperature overnight. The reaction mixture was diluted by ethyl acetate (100 mL) and washed with water (50 mL) three times, and the organic layer was dried over anhydrous sodium sulfate. The solvent was removed to yield N-(5-bromopyridin-2-yl)cyclopropanecarboxamide as a yellow solid, which used in the next step without further purification.

### Step 2: N-(5-(1-ethoxyvinyl)pyridin-2-yl)cyclopropanecarboxamide

To a solution of N-(5-bromopyridin-2-yl)cyclopropanecarboxamide (2 g, 8.29 mmol) in 1,4-dioxane (15 mL) was added tributyl(1-ethoxyvinyl)stannane (3.6 g, 9.95 mmol), Pd(PPh₃)₄ (1.0 g, 0.830 mmol). The resulting mixture was maintained under nitrogen and stirred at 100° C. overnight. After cooling to room temperature, the reaction was quenched with water (50 mL), then extracted with ethyl acetate (3×50 mL). The organic layers were combined, dried over anhydrous sodium sulfate, filtered and concentrated under vacuum. The residue was purified by silica gel chromatography (0→30% ethyl acetate/petroleum ether) to yield N-(5-(1-ethoxyvinyl)pyridin-2-yl)cyclopropanecarboxamide as a light yellow solid. LC/MS: mass calculated for C₁₃H₁₆N₂O₂: 232.12, measured: 233.1 [M+H]⁺.

### Step 3: N-(5-Acetylpyridin-2-yl)cyclopropanecarboxamide

To a solution of N-(5-(1-ethoxyvinyl)pyridin-2-yl)cyclopropanecarboxamide (1.1 g, 4.736 mmol) in THE (15 mL) was added 2N HCl (1.5 mL). The mixture stirred at room temperature for 2 h. The resulting mixture was extracted with ethyl acetate (3×20 mL). The organic layers were combined, dried over anhydrous sodium sulfate, filtered and concentrated. The residue was purified by silica gel chromatography (0→30% ethyl acetate/petroleum ether) to yield the N-(5-acetylpyridin-2-yl)cyclopropanecarboxamide as a yellow solid. LC/MS: mass calculated for C₁₁H₁₂N₂O₂: 204.09, measured: 205.1 [M+H]⁺.

### Step 4: N-(5-(2-Bromoacetyl)pyridin-2-yl)cyclopropanecarboxamide

To a solution of N-(5-acetylpyridin-2-yl)cyclopropanecarboxamide (1 g, 4.89 mmol), hydrobromic acid, HOAc solution 33% (2.4 g, 9.73 mmol) in HOAc (20 mL) was added pyridinium tribromide (1.4 g, 9.79 mmol) at 0° C. The resulting mixture was stirred at room temperature for 2 h. The mixture was concentrated, diluted with water (100 mL), then extracted with ethyl acetate (4×100 mL). The organic layers were combined, dried over anhydrous sodium sulfate, filtered and concentrated. The residue was purified by silica gel chromatography (0-30% ethyl acetate/petroleum ether) to yield the N-(5-(2-bromoacetyl)pyridin-2-yl)cyclopropanecarboxamide as a white solid. LC/MS: mass calculated for C₁₁H₁₁BrN₂O₂: 282.00, measured: 282.9 [M+H]⁺.

### Intermediate Example Y: 1-(6-amino-5-fluoro-2-methylpyridin-3-yl)-2-bromoethan-1-one

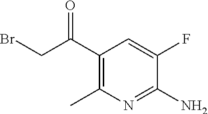

### Step 1: 5-bromo-3-fluoro-6-methylpyridin-2-amine

To a solution of 3-fluoro-6-methylpyridin-2-amine (2 g, 15.86 mmol, 1.0 equiv) in DMF (20 mL) was added NBS (3.1 g, 17.44 mmol, 1.1 equiv) at 0° C. The reaction mixture was warmed to room temperature and stirred for 2 h, diluted with water and extracted with ethyl acetate, the organic layer was washed with brine and dried over anhydrous Na₂SO₄. The solvent was removed under vacuum and resulting residue was purified by flash column chromatography on silica gel (EA/PE, 1-40%) to yield 5-bromo-3-fluoro-6-methylpyridin-2-amine as a yellow solid.

### Step 2: 1-(6-amino-5-fluoro-2-methylpyridin-3-yl)ethan-1-one

To a solution of 5-bromo-3-fluoro-6-methylpyridin-2-amine (1.3 g, 6.34 mmol, 1 equiv) and tributyl(1-ethoxyvinyl)tin (2.4 ml, 6.98 mmol, 1.1 equiv) in 1,4-dioxane (15 mL) was added Pd(PPh₃)₄ (366 mg, 0.32 mmol, 0.05 equiv) under N₂. The reaction mixture was stirred at 100° C. overnight. After the mixture was cooled, 1 M HCl (20 mL) was added and stirred for 20 min. The reaction mixture was partitioned between water and ethyl acetate. The organic layer was separated, washed with brine and dried over anhydrous Na₂SO₄. The solvent was removed under vacuum to yield 1-(6-amino-5-fluoro-2-methylpyridin-3-yl)ethan-1-one as a yellow solid.

### Step 3: 1-(6-amino-5-fluoro-2-methylpyridin-3-yl)-2-bromoethan-1-one

A mixture of 1-(6-amino-5-fluoro-2-methylpyridin-3-yl)ethan-1-one (720 mg, 4.28 mmol, 1.0 equiv), HBr/AcOH (1.4 ml, 8.56 mmol, 2.0 equiv) and pyridinium tribromide (1.4 g, 4.28 mmol, 1.0 equiv) in AcOH (6 mL) was stirred at room temperature for 2 h. The reaction mixture was concentrated and partitioned between water and ethyl acetate. The organic layer was washed with water and aqueous NaHCO₃ and dried over anhydrous Na₂SO₄. The filtrate was concentrated to yield 1-(6-amino-5-fluoro-2-methylpyridin-3-yl)-2-bromoethan-1-one as a yellow solid.

### Intermediate Example Z: 2-Bromo-1-(2-(trifluoromethyl)pyridin-4-yl)ethan-1-one

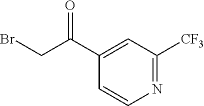 [SMILES: O=C(CBr)c1ccnc(C(F)(F)F)c1]

### Step 1: 4-(1-Ethoxyvinyl)-2-(trifluoromethyl)pyridine

To a solution of 4-bromo-2-(trifluoromethyl)pyridine (100 mg, 0.44 mmol, 1 equiv) and tributyl(1-ethoxyvinyl)stannane (0.16 mL, 0.48 mmol, 1.1 equiv) in 1,4-dioxane (10 mL) was added tetrakis(triphenylphosphine)palladium (26 mg, 0.022 mmol, 0.05 equiv) under an atmosphere of nitrogen. The reaction mixture was stirred 8 h at 100° C. The reaction mixture was used in the next step without further purification or isolation. LC/MS: mass calculated for C₁₀H₁₀F₃NO: 217.07, measured: 218.00 [M+H]⁺.

### Step 2: 2-Bromo-1-(2-(trifluoromethyl)pyridin-4-yl)ethan-1-one

To a solution of 4-(1-ethoxyvinyl)-2-(trifluoromethyl)pyridine (2 g, 9.20 mmol, 1 equiv) in 1,4-dioxane (30 mL) and water (20 mL) was added NBS (1.6 g, 9.20 mmol, 1 equiv). The mixture was stirred 2 h at room temperature. The mixture was quenched with water, then extracted with ethyl acetate. The combined organic layer was dried over anhydrous sodium sulfate and concentrated under vacuum. The residue was purified by silica gel chromatography with EA/PE (0→80%) to yield 2-bromo-1-(2-(trifluoromethyl)pyridin-4-yl)ethan-1-one as off-white solid. LC/MS: mass calculated for C₈H₅BrF₃NO: 266.95, measured: 269.95 [M+H+2]⁺.

### Synthesis Examples: Compounds of Formula (I)

### Example 1: (1R*,3S*)-7-(3-Chloro-2-fluoro-6-(1H-tetrazol-1-yl)phenyl)-3-(5-(3-fluoro-2-(hydroxymethyl)pyridin-4-yl)-1H-imidazol-2-yl)-1-methyl-2,3-dihydroindolizin-5(1H)-one

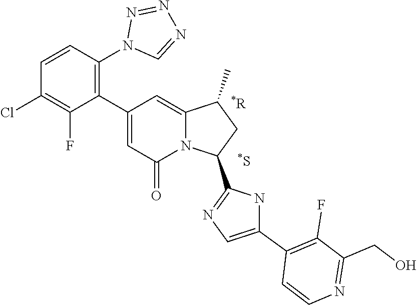 [SMILES: C[C@@H]1C[C@@H](c2ncc(-c3ccnc(CO)c3F)[nH]2)n2c1cc(-c1c(-n3cnnn3)ccc(Cl)c1F)cc2=O]

LC/MS: mass calculated for C₂₅H₁₉ClF₂N₈O₂:536.13, measured (ES, m/z): 537.20 [M+H]⁺. ¹H NMR (300 MHz, DMSO-d₆) δ 12.60 (s, 1H), 9.68 (s, 1H), 8.33 (d, J=5.1 Hz, 1H), 7.93-8.12 (m, 1H), 7.81 (t, J=5.6 Hz, 1H), 7.75 (dd, J=8.7, 1.6 Hz, 1H), 7.55-7.65 (m, 1H), 6.18 (s, 1H), 6.05 (s, 1H), 5.68 (d, J=8.6 Hz, 1H), 5.13-5.44 (m, 1H), 4.63 (d, J=2.3 Hz, 2H), 3.57-3.77 (m, 1H), 2.09-2.32 (m, 1H), 1.91-2.08 (m, 1H), 1.21 (d, J=6.8 Hz, 3H). ¹⁹F NMR (282 MHz, DMSO-d₆) δ −113.10, −130.09

### Example 2: (1 S*,3S*)-7-(3-Chloro-2-fluoro-6-(1H-tetrazol-1-yl)phenyl)-3-(5-(3-fluoro-2-(hydroxymethyl)pyridin-4-yl)-1H-imidazol-2-yl)-1-methyl-2,3-dihydroindolizin-5(1H)-one

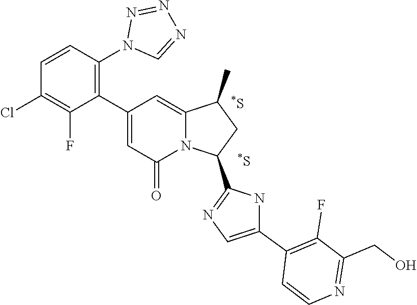 [SMILES: C[C@H]1C[C@@H](c2ncc(-c3ccnc(CO)c3F)[nH]2)n2c1cc(-c1c(-n3cnnn3)ccc(Cl)c1F)cc2=O]

### Step 1: 2-(((Tert-butyldimethylsilyl)oxy)methyl)-3-fluoropyridine

To a mixture of (3-fluoropyridin-2-yl)methanol (20 g, 157.34 mmol, 1.0 equiv.) in dichloromethane (200 mL) was added 2,6-lutidine (54.98 mL, 472.01 mmol, 3.0 equiv.) and TBSOTf (54.20 mL, 236.01 mmol, 1.5 equiv.). The solution was stirred at room temperature for 1 h. The reaction was diluted with water and extracted with DCM twice. The combined organic layers were washed with brine, dried over Na₂SO₄ and concentrated under vacuum. The residue was purified by silica gel chromatography (0→30% ethyl acetate/petroleum ether) to yield 2-(((tert-butyldimethylsilyl)oxy)methyl)-3-fluoropyridine as yellow oil. LC/MS: mass calculated for C₁₂H₂₀FNOSi: 241.13, measured (ES, m/z): 242.10 [M+H]⁺.

### Step 2: 2-(((Tert-butyldimethylsilyl)oxy)methyl)-3-fluoro-4-iodopyridine

To a solution of 2-(((tert-butyldimethylsilyl)oxy)methyl)-3-fluoropyridine (34.7 g, 0.14 mol, 1.0 equiv.) in dry tetrahydrofuran (350 mL) under nitrogen was added n-butyllithium (63 mL, 0.16 mol, 1.1 equiv.) at −78° C. and the solution was stirred for 1.0 h at this temperature under N₂. A solution of iodine (40 g, 0.16 mol, 1.1 equiv.) in dry toluene (50 mL) was added dropwise at −78° C. and the solution was stirred at −78° C. for 1 h. The reaction mixture was warmed and stirred at room temperature for 1 h. The solution was quenched with sat. NH₄Cl (aq.) and diluted with water. The mixture was extracted with ethyl acetate twice. The combined organic layers were washed by Na₂S₂O₃, washed with brine, dried over Na₂SO₄, concentrated under vacuum. The residue was purified by silica gel chromatography (0→20% ethyl acetate/petroleum ether) to yield 2-(((tert-butyldimethylsilyl)oxy)methyl)-3-fluoro-4-iodopyridine as an off-white solid. LC/MS: mass calculated for C₁₂H₁₉FINOSi: 367.03, measured (ES, m/z): 368.00 [M+H]⁺.

### Step 3: 2-(((Tert-butyldimethylsilyl)oxy)methyl)-4-(1-ethoxyvinyl)-3-fluoropyridine

To a mixture of 2-(((tert-butyldimethylsilyl)oxy)methyl)-3-fluoro-4-iodopyridine (15 g, 40.84 mmol, 1.0 equiv.) in 1,4-dioxane (150 ml) was added tributyl(1-ethoxyvinyl)stannane (27.60 ml, 81.68 mmol, 2.0 equiv.) and Pd(PPh₃)₄ (2.36 g, 2.04 mmol, 0.05 equiv.). The flask was evacuated and flushed back three times with nitrogen. The solution was stirred at 100° C. for 12 h under nitrogen. After cooling to room temperature, water was added and the reaction mixture was extracted with ethyl acetate twice. The combined organic layers were washed with brine, dried over Na₂SO₄ and concentrated under vacuum. The residue was purified by Al₂O₃ column (0→10% ethyl acetate/petroleum ether) to yield 2-(((tert-butyldimethylsilyl)oxy)methyl)-4-(1-ethoxyvinyl)-3-fluoropyridine as a yellow oil. LC/MS: mass calculated for C₁₆H₂₆FNO₂Si: 311.17, measured (ES, m/z): 312.10 [M+H]⁺

### Step 4: 2-Bromo-1-(2-(((tert-butyldimethylsilyl)oxy)methyl)-3-fluoropyridin-4-yl)ethan-1-one

To a mixture of 2-(((tert-butyldimethylsilyl)oxy)methyl)-4-(1-ethoxyvinyl)-3-fluoropyridine (500 mg, 1.61 mmol, 1.0 equiv.) in tetrahydrofuran (6 ml) and water (2 ml) was added N-bromosuccinimide (228.6 mg, 1.28 mmol, 0.8 equiv.). The solution was stirred at room temperature for 0.5 h. The reaction mixture was added water and extracted with ethyl acetate twice. The combined organic layers were washed with brine, dried over Na₂SO₄ and concentrated under vacuum. This resulted in 2-bromo-1-(2-(((tert-butyldimethylsilyl)oxy)methyl)-3-fluoropyridin-4-yl)ethan-1-one as a yellow oil. LC/MS: mass calculated for C₁₄H₂₁BrFNO₂Si: 361.05, measured (ES, m/z): 363.95 [M+H+2]⁺.

### Step 5: 2-(2-(((Tert-butyldimethylsilyl)oxy)methyl)-3-fluoropyridin-4-yl)-2-oxoethyl 7-(6-amino-3-chloro-2-fluorophenyl)-1-methyl-5-oxo-1,2,3,5-tetrahydroindolizine-3-carboxylate

To a solution of 7-(6-amino-3-chloro-2-fluorophenyl)-1-methyl-5-oxo-1,2,3,5-tetrahydroindolizine-3-carboxylic acid (200 mg, 0.59 mmol, 1.0 equiv.) in N,N-dimethylformamide (7.0 mL) was added potassium carbonate (90.29 mg, 0.65 mmol, 1.1 equiv.). Ten minutes later, to the solution was added 2-bromo-1-(2-(((tert-butyldimethylsilyl)oxy)methyl)-3-fluoropyridin-4-yl)ethan-1-one (430 mg, 1.19 mmol, 2.0 equiv.). The reaction mixture was stirred at room temperature for 1 h. To the reaction mixture was added water, and the resulting mixture was extracted with ethyl acetate twice. The combined organic layer was washed with brine, dried over Na₂SO₄ and concentrated under vacuum. The residue was purified by silica gel chromatography (0→10% methanol/dichloromethane) to yield 2-(2-(((tert-butyldimethylsilyl)oxy)methyl)-3-fluoropyridin-4-yl)-2-oxoethyl 7-(6-amino-3-chloro-2-fluorophenyl)-1-methyl-5-oxo-1,2,3,5-tetrahydroindolizine-3-carboxylate as yellow solid. LC/MS: mass calculated for C₃₀H₃₄ClF₂N₃O₅Si: 617.19, measured (ES, m/z): 618.10 [M+H]⁺.

### Step 6: 7-(6-Amino-3-chloro-2-fluorophenyl)-3-(5-(2-(((tert-butyldimethylsilyl)oxy)methyl)-3-fluoropyridin-4-yl)-1H-imidazol-2-yl)-1-methyl-2,3-dihydroindolizin-5(1H)-one

To a mixture of 2-(2-(((tert-butyldimethylsilyl)oxy)methyl)-3-fluoropyridin-4-yl)-2-oxoethyl 7-(6-amino-3-chloro-2-fluorophenyl)-1-methyl-5-oxo-1,2,3,5-tetrahydroindolizine-3-carboxylate (280 mg, 0.45 mmol, 1.0 equiv.) in toluene (5 ml) and acetic acid (0.5 mL) was added ammonium acetate (349 mg, 4.53 mmol, 10.0 equiv.). The reaction mixture was stirred at 100° C. for 1.0 h. The solution was concentrated under vacuum. The residue was purified by reverse phase chromatography on C18 (330 g, ACN/H₂O (0.05% CF₃COOH): 0→28%) to yield 7-(6-amino-3-chloro-2-fluorophenyl)-3-(5-(2-(((tert-butyldimethylsilyl)oxy)methyl)-3-fluoropyridin-4-yl)-1H-imidazol-2-yl)-1-methyl-2,3-dihydroindolizin-5(1H)-one as a yellow solid. LC/MS: mass calculated for C₃₀H₃₄ClF₂N₅O₂Si: 597.21, measured (ES, m/z): 598.25 [M+H]⁺.

### Step 7: 3-(5-(2-(((Tert-butyldimethylsilyl)oxy)methyl)-3-fluoropyridin-4-yl)-1H-imidazol-2-yl)-7-(3-chloro-2-fluoro-6-(1H-tetrazol-1-yl)phenyl)-1-methyl-2,3-dihydroindolizin-5(1H)-one

To a mixture of 7-(6-amino-3-chloro-2-fluorophenyl)-3-(5-(2-(((tert-butyldimethylsilyl)oxy)methyl)-3-fluoropyridin-4-yl)-1H-imidazol-2-yl)-1-methyl-2,3-dihydroindolizin-5(1H)-one (180 mg, 0.30 mmol, 1.0 equiv.) in acetic acid (2 mL) were added trimethoxymethane (0.13 mL, 1.20 mmol, 4.0 equiv.) and azidotrimethylsilane (0.16 mL, 1.20 mmol, 4.0 equiv.). The solution was stirred at room temperature overnight. The mixture was concentrated under vacuum to yield 3-(5-(2-(((tert-butyldimethylsilyl)oxy)methyl)-3-fluoropyridin-4-yl)-1H-imidazol-2-yl)-7-(3-chloro-2-fluoro-6-(1H-tetrazol-1-yl)phenyl)-1-methyl-2,3-dihydroindolizin-5(1H)-one as a yellow solid. LC/MS: mass calculated for C₃₁H₃₃ClF₂N₈O₂Si: 650.22, measured (ES, m/z): 651.15 [M+H]⁺.

### Step 8: (1S,3S)-7-(3-Chloro-2-fluoro-6-(1H-tetrazol-1-yl)phenyl)-3-(5-(3-fluoro-2-(hydroxymethyl)pyridin-4-yl)-1H-imidazol-2-yl)-1-methyl-2,3-dihydroindolizin-5(1H)-one

To a mixture of 3-(5-(2-(((tert-butyldimethylsilyl)oxy)methyl)-3-fluoropyridin-4-yl)-1H-imidazol-2-yl)-7-(3-chloro-2-fluoro-6-(1H-tetrazol-1-yl)phenyl)-1-methyl-2,3-dihydroindolizin-5(1H)-one (170 mg, 0.26 mmol, 1.0 equiv.) in tetrahydrofuran (2 mL) was added triethylamine trihydrofluoride (1.0 mL, 6.14 mmol). The solution was stirred at room temperature for 1.0 h. The solution was concentrated under vacuum. The residue was purified by reverse phase chromatography on C18 (80 g, ACN/H₂O (0.05% CF₃COOH): 0→40%) to yield a residue, which was further purified by prep-HPLC (Column: Xselect CSH OBD Column 30*150 mm 5 um; Mobile Phase A:Water (0.1% FA), Mobile Phase B:ACN; Flow rate: 60 mL/min; Gradient: 17 B to 27 B in 12 min; 254/220 nm) and Prep-Chiral-HPLC (Column: (R,R) WHELK-01, 4.6*50 mm, 3.5 um; Mobile Phase: MtBE (0.1% DEA): EtOH=50:50; Flow rate: 1 mL/min) to yield (1S,3S)-7-(3-chloro-2-fluoro-6-(1H-tetrazol-1-yl)phenyl)-3-(5-(3-fluoro-2-(hydroxymethyl)pyridin-4-yl)-1H-imidazol-2-yl)-1-methyl-2,3-dihydroindolizin-5(1H)-one as a white solid.

LC/MS: mass calculated for C₂₅H₁₉ClF₂N₈O₂:536.13, measured (ES, m/z): 537.10 [M+H]⁺. ¹H NMR (400 MHz, DMSO-d₆) δ 12.62 (s, 1H), 9.67 (s, 1H), 8.32 (d, J=5.0 Hz, 1H), 7.96-8.08 (m, 1H), 7.83 (t, J=5.4 Hz, 1H), 7.74 (dd, J=8.7, 1.6 Hz, 1H), 7.64 (d, J=3.9 Hz, 1H), 6.12 (s, 1H), 6.05 (s, 1H), 5.51-5.63 (m, 1H), 5.26-5.33 (m, 1H), 4.63 (s, 2H), 3.40-3.42 (m, 1H), 2.70-2.86 (m, 1H), 1.92-2.09 (m, 1H), 1.32 (d, J=7.3 Hz, 3H). ¹⁹F NMR (376 MHz, DMSO-d₆) δ −113.09, −130.19

### Example 3: (R)-3′-(5-(6-Amino-2-fluoropyridin-3-yl)-1H-imidazol-2-yl)-7′-(5-chloro-2-(1H-tetrazol-1-yl)phenyl)-2′,3′-dihydro-5′H-spiro[cyclopropane-1,1′-indolizin]-5′-one

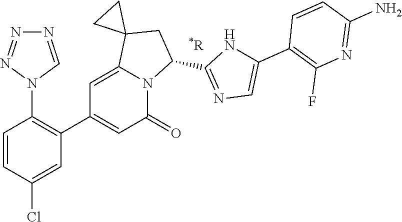 [SMILES: Nc1ccc(-c2cnc([C@H]3CC4(CC4)c4cc(-c5cc(Cl)ccc5-n5cnnn5)cc(=O)n43)[nH]2)c(F)n1]

### Step 1: 4-(Benzyloxy)-2,6-dibromopyridine

To a solution of benzyl alcohol (17.2 mL, 166.3 mmol, 1.05 equiv.) in 1,4-dioxane (167 mL) was added sodium hydride (6.6 g, 166.3 mmol, 1.05 equiv. ca 60% dispersion in oil) at 0° C. The resulting mixture was stirred at room temperature for 1 h to yield the suspension of sodium phenylmethanolate.

To a solution of 2,4,6-tribromopyridine (50.0 g, 158.3 mmol, 1.0 equiv.) in anhydrous N,N-dimethylformamide (833 mL) was added dropwise the suspension of sodium phenylmethanolate at −20° C. The resulting mixture was warmed to room temperature slowly. After 2 h, the reaction was cooled to 0° C. and quenched with water (833 mL). The mixture was filtered. The filter cake was dissolved in petroleum ether (120 mL) and stirred for 30 min. The filter cake was collected, dried to yield the 4-(benzyloxy)-2,6-dibromopyridine as a white solid. LC/MS (ES, m/z): mass calculated for C₁₂H₉Br₂NO: 340.91, measured: 343.90 [M+H+2]⁺.

### Step 2: 4-(Benzyloxy)-2-bromo-6-((4-methoxybenzyl)oxy)pyridine

To a solution of (4-methoxyphenyl)methanol (17.6 g, 128.0 mmol, 1.05 equiv.) in 1,4-dioxane (400 mL) was added sodium hydride (60%, 5.3 g, 118.4 mmol, 1.1 equiv.) under nitrogen at 0° C. After 30 minutes, 4-(benzyloxy)-2,6-dibromopyridine (41.8 g, 121.9 mmol, 1.0 equiv.) was added. The resulting mixture was stirred 80° C. for 2 h. After cooling to room temperature, to the reaction mixture was added water (200 mL), and the resulting mixture was extracted with ethyl acetate (3×200 mL). The organic layers were combined, dried over Na₂SO₄, filtered and concentrated to yield the 4-(benzyloxy)-2-bromo-6-((4-methoxybenzyl)oxy)pyridine as yellow oil. ¹H NMR (300 MHz, DMSO-d₆) δ 7.16-7.52 (m, 7H), 6.83-7.01 (m, 3H), 6.50 (d, J=1.9 Hz, 1H), 5.18 (d, J=4.9 Hz, 4H), 3.75 (s, 3H).

### Step 3: 4-(Benzyloxy)-6-bromopyridin-2-ol

To a solution of 4-(benzyloxy)-2-bromo-6-(4-methoxybenzyloxy)pyridine (60.5 g, 151.1 mmol, 1.0 equiv.) in dichloromethane (120 mL) was added trifluoroacetic acid (40 mL). The resulting mixture was stirred at room temperature for 3 hours. After the excess solvent was roto-evaporated, sat. NaHCO₃ (aq., 100 mL) was added. The resulting mixture was stirred for 1 h. The solid was filtered and washed with water (200 mL) and petroleum ether (300 mL) respectively to yield the 4-(benzyloxy)-6-bromopyridin-2-ol as white solid. LC/MS (ES, m/z): mass calculated for C₁₂H₁₀BrNO₂: 278.99, measured: 280.00[M+H]⁺.

### Step 4: Ethyl 2-(4-(benzyloxy)-6-bromo-2-oxopyridin-1(2H)-yl) acetate

To a solution of 4-(benzyloxy)-6-bromopyridin-2-ol (39 g, 140.4 mmol, 1.0 equiv.) in N,N-dimethylformamide (50 mL) and 1,2-dimethoxyethane (250 mL) was added sodium hydride (60%, 5.9 g, 147.4 mmol, 1.05 eq.) under nitrogen at 0° C. After 1 h, lithium bromide (24.4 g, 280.8 mmol, 2.0 equiv.) was added. The resulting mixture was stirred at room temperature for 1 hour. The reaction mixture was cooled to 0° C., and then ethyl 2-bromoacetate (31 mL, 280.8 mmol, 2.0 equiv.) was added. The resulting mixture was heated to 65° C. for 2 h under nitrogen. After cooling to 0° C., to the mixture was added ice water (500 mL). The mixture was filtered. The filter cake was added in a mixture solvents of ethyl acetate (75 mL) and petroleum ether (25 mL) and stirred at room temperature for 0.5 h. The mixture was filtered. The filter cake was rinsed with water (50 mL) to yield the ethyl 2-(4-(benzyloxy)-6-bromo-2-oxopyridin-1(2H)-yl) acetate as a white solid. LC/MS (ES, m/z): mass calculated for C₁₆H₁₆BrNO₄:365.03, measured: 366.1 [M+H]⁺.

### Step 5: Ethyl 2-(4-(benzyloxy)-6-bromo-2-oxopyridin-1(2H)-yl)pent-4-enoate

To a mixture of ethyl 2-(4-(benzyloxy)-6-bromo-2-oxopyridin-1(2H)-yl)acetate (20 g, 54.6 mmol, 1.0 equiv.) in THE (300 mL) at −70° C. under N₂ was added LiHMDS (64.5 mL, 65.5 mmol, 1.2 equiv.). After 1 hour, 3-iodoprop-1-ene (9.6 mL, 57.3 mmol, 1.05 equiv.) was added. The reaction was warmed to r.t and stirred for 3 h. The reaction was quenched with saturated NH₄Cl solution and extracted with ethyl acetate (3×200 mL). The combined extracts were washed with water and brine, dried over anhydrous sodium sulfate, concentration and chromatography on ethyl acetate/petroleum ether (1→50%) to yield ethyl 2-(4-(benzyloxy)-6-bromo-2-oxopyridin-1(2H)-yl)pent-4-enoate as a yellow solid. LC/MS (ES, m/z): mass calculated for C₁₉H₂₀BrNO₄: 405.06, measured: 405.95 [M+H]⁺.

### Step 6: Ethyl 7-(benzyloxy)-1-methylene-5-oxo-1,2,3,5-tetrahydroindolizine-3-carboxylate

To a solution of ethyl 2-(4-(benzyloxy)-6-bromo-2-oxopyridin-1(2H)-yl)pent-4-enoate (20.0 g, 49.2 mmol, 1.0 equiv.) and triphenylphosphine (1.3 g, 4.9 mmol, 0.1 equiv.) in CH₃CN (230 mL) with diacetoxypalladium (552 mg, 2.5 mmol, 0.05 equiv.) was added triethylamine (13.7 mL, 98.5 mmol, 2.0 equiv.).

The resulting mixture was stirred at 80° C. for 5 h, concentrated under reduced pressure and the residue was purified by flash column chromatography on silica gel with ethyl acetate/petroleum ether (1→80%) to yield ethyl 7-(benzyloxy)-1-methylene-5-oxo-1,2,3,5-tetrahydroindolizine-3-carboxylate as a yellow solid. LC/MS (ES, m/z): mass calculated for C₁₉H₁₉NO₄: 325.13, measured: 326.10 [M+H]⁺.

### Step 7: Ethyl 7′-(benzyloxy)-5′-oxo-2-(trimethylsilyl)-2′,3′-dihydro-5′H-spiro[cyclopropane-1,1′-indolizine]-3′-carboxylate

To a mixture of ethyl 7-(benzyloxy)-1-methylene-5-oxo-1,2,3,5-tetrahydroindolizine-3-carboxylate (5.5 g, 16.9 mmol, 1.0 equiv.) and CuPC (974 mg, 1.69 mmol, 0.1 equiv.) in DCE (60 mL) was added TMSCHN₂ (42.3 mL, 84.5 mmol, 5.0 equiv.) at room temperature under N₂ and the solution was stirred for 2 h at 85° C. The CuPC was then filtered out and the mixture was purified by silica gel chromatography (0→5% methanol/dichloromethane) to yield ethyl 7′-(benzyloxy)-5′-oxo-2-(trimethylsilyl)-2′,3′-dihydro-5′H-spiro[cyclopropane-1,1′-indolizine]-3′-carboxylate as a yellow oil. LC/MS (ES, m/z): mass calculated for C₂₃H₂₉NO₄Si: 411.19, measured: 412.20 [M+H]⁺.

### Step 8: Ethyl 7′-hydroxy-5′-oxo-2-(trimethylsilyl)-2′,3′-dihydro-5′H-spiro[cyclopropane-1,1′-indolizine]-3′-carboxylate

Palladium-carbon (3.4 g, 10%) was put into a 100 mL round-bottom flask and methanol (20 mL) was added to the flask to soak the palladium-carbon. Ethyl 7′-(benzyloxy)-5′-oxo-2-(trimethylsilyl)-2′,3′-dihydro-5′H-spiro[cyclopropane-1,1′-indolizine]-3′-carboxylate (2.6 g, 6.3 mmol, 1.0 equiv.) was dissolved in methanol (10 mL) and the resulting solution was added to above mixture for hydrogenation under H₂ (1 atm). After 2 h, the reaction was filtrated and the filtrate was concentrated under vacuum to yield ethyl 7′-hydroxy-5′-oxo-2-(trimethylsilyl)-2′,3′-dihydro-5′H-spiro[cyclopropane-1,1′-indolizine]-3′-carboxylate as a yellow oil. LC/MS (ES, m/z): mass calculated for C₁₆H₂₃NO₄Si: 321.14, measured: 322.20 [M+H]⁺.

### Step 9: Ethyl 5′-oxo-7′-(((trifluoromethyl)sulfonyl)oxy)-2-(trimethylsilyl)-2′,3′-dihydro-5′H-spiro[cyclopropane-1,1′-indolizine]-3′-carboxylate

To a mixture of ethyl 7′-hydroxy-5′-oxo-2-(trimethylsilyl)-2′,3′-dihydro-5′H-spiro[cyclopropane-1,1′-indolizine]-3′-carboxylate (2.0 g, 6.10 mmol, 1.0 equiv.) and triethylamine (2.5 mL, 18.20 mmol, 3.0 equiv.) in dichloromethane (20 mL) was added 1,1,1-trifluoro-N-phenyl-N-((trifluoromethyl)sulfonyl)methane sulfonamide (2.6 g, 7.28 mmol, 1.2 equiv.) at 0° C. and the solution was stirred for 2 h at room temperature. The mixture was diluted with H₂O and extracted with dichloromethane twice. The combined organic layers was washed with brine, dried over Na₂SO₄, concentrated and purified by silica gel chromatography (0→70% ethyl acetate/petroleum ether) to yield ethyl 5′-oxo-7′-(((trifluoromethyl)sulfonyl)oxy)-2-(trimethylsilyl)-2′,3′-dihydro-5′H-spiro[cyclopropane-1,1′-indolizine]-3′-carboxylate as a yellow oil. LC/MS (ES, m/z): mass calculated for C₁₇H₂₂F₃NO₆SSi: 453.09, measured: 454.25 [M+H]⁺.

### Step 10: Ethyl 7′-(2-amino-5-chlorophenyl)-5′-oxo-2-(trimethylsilyl)-2′,3′-dihydro-5′H-spiro[cyclopropane-1,1′-indolizine]-3′-carboxylate

To a solution of ethyl 5′-oxo-7′-(((trifluoromethyl)sulfonyl)oxy)-2-(trimethylsilyl)-2′,3′-dihydro-5′H-spiro[cyclopropane-1,1′-indolizine]-3′-carboxylate (1.3 g, 2.87 mmol, 1.0 equiv.) and 4-chloro-2-(4,4,5,5-tetramethyl-1,3,2-dioxaborolan-2-yl)aniline (1.45 g, 5.73 mmol, 2.0 equiv.) in 1,4-dioxane (50 mL), water (10 mL) was added tetrakis(triphenylphosphine)palladium (331 mg, 0.29 mmol, 0.1 equiv.) and cesium fluoride (1.2 g, 8.60 mmol, 3.0 equiv.) under N₂. The reaction mixture was stirred at 100° C. for 2 h. Then cooled to r.t and quenched with water. The mixture was extracted with ethyl acetate. The organic layers were combined, dried over Na₂SO₄, filtered and concentrated. The residue was purified by silica gel column (30→100% EA/PE) to yield of ethyl 7′-(2-amino-5-chlorophenyl)-5′-oxo-2-(trimethylsilyl)-2′,3′-dihydro-5′H-spiro[cyclopropane-1,1′-indolizine]-3′-carboxylate as brown oil. LC/MS (ES, m/z): mass calculated for C₂₂H₂₇ClN₂O₃Si: 430.15, measured: 431.15 [M+H]⁺.

### Step 11: 7′-(2-Amino-5-chlorophenyl)-5′-oxo-2′,3′-dihydro-5′H-spiro[cyclopropane-1,1′-indolizine]-3′-carboxylic acid

A mixture of ethyl 7′-(2-amino-5-chlorophenyl)-5′-oxo-2-(trimethylsilyl)-2′,3′-dihydro-5′H-spiro[cyclopropane-1,1′-indolizine]-3′-carboxylate (0.94 g, 2.18 mmol, 1.0 equiv.) and TBAF (10 mL, 1 M in THF) was stirred overnight at 70° C. The solvent was evaporated under vacuum and the residue was purified by reverse phase chromatography on C18 column (0→50% ACN/H₂O) to yield 7′-(2-amino-5-chlorophenyl)-5′-oxo-2′,3′-dihydro-5′H-spiro[cyclopropane-1,1′-indolizine]-3′-carboxylic acid as a yellow solid. LC/MS (ES, m/z): mass calculated for C₁₇H₁₁ClN₂O₃: 330.08, measured: 331.05 [M+H]⁺.

### Step 12: 2-(6-Acetamido-2-fluoropyridin-3-yl)-2-oxoethyl 7′-(2-amino-5-chlorophenyl)-5′-oxo-2′,3′-dihydro-5′H-spiro[cyclopropane-1,1′-indolizine]-3′-carboxylate

To a solution of 7′-(2-amino-5-chlorophenyl)-5′-oxo-2′,3′-dihydro-5′H-spiro[cyclopropane-1,1′-indolizine]-3′-carboxylic acid (150 mg, 0.45 mmol, 1.0 equiv.) in DMF (5 mL) was added potassium carbonate (125 mg, 0.91 mmol, 2.0 equiv.). After the reaction mixture was stirred for 0.5 h at room temperature, N-(5-(2-bromoacetyl)-6-fluoropyridin-2-yl)acetamide (250 mg, 0.91 mmol, 2.0 equiv.) was added. The reaction mixture was stirred for 2 h at room temperature. The residue was purified by reverse column chromatography with CH₃CN/0.05% TFA water (5%→70%) to yield 2-(6-acetamido-2-fluoropyridin-3-yl)-2-oxoethyl 7′-(2-amino-5-chlorophenyl)-5′-oxo-2′,3′-dihydro-5′ H-spiro[cyclopropane-1,1′-indolizine]-3′-carboxylate as a yellow solid. LC/MS (ES, m/z): mass calculated for C₂₆H₂₂ClFN₄O₅: 524.13, measured: 525.10 [M+H]⁺.

### Step 13: N-(5-(2-(7′-(2-amino-5-chlorophenyl)-5′-oxo-2′,3′-dihydro-5′H-spiro[cyclopropane-1,1′-indolizin]-3′-yl)-1H-imidazol-5-yl)-6-fluoropyridin-2-yl)acetamide

To a solution of 2-(6-acetamido-2-fluoropyridin-3-yl)-2-oxoethyl 7′-(2-amino-5-chlorophenyl)-5′-oxo-2′,3′-dihydro-5′H-spiro[cyclopropane-1,1′-indolizine]-3′-carboxylate (0.2 g, 0.38 mmol, 1.0 equiv.) in toluene (5 mL) and acetic acid (0.25 mL) was added ammonium acetate (0.59 g, 7.62 mmol, 20.0 equiv.). The reaction mixture was stirred at 100° C. for 1 h, then concentrated under vacuum. The residue was purified by silica gel chromatography (0→10% DCM/MeOH) to yield N-(5-(2-(7′-(2-amino-5-chlorophenyl)-5′-oxo-2′,3′-dihydro-5′H-spiro[cyclopropane-1,1′-indolizin]-3′-yl)-1H-imidazol-5-yl)-6-fluoropyridin-2-yl)acetamide as a brown solid. LC/MS (ES, m/z): mass calculated for C₂₆H₂₂ClFN₆O₂: 504.15, measured: 505.15 [M+H]⁺.

### Step 14: N-(5-(2-(7′-(5-chloro-2-(1H-tetrazol-1-yl)phenyl)-5′-oxo-2′,3′-dihydro-5′H-spiro[cyclopropane-1,1′-indolizin]-3′-yl)-1H-imidazol-5-yl)-6-fluoropyridin-2-yl)acetamide

To a solution of N-(5-(2-(7′-(2-amino-5-chlorophenyl)-5′-oxo-2′,3′-dihydro-5′H-spiro[cyclopropane-1,1′-indolizin]-3′-yl)-1H-imidazol-5-yl)-6-fluoropyridin-2-yl)acetamide (0.12 g, 0.24 mmol, 1.0 equiv.) in acetic acid (10 mL) were added trimethoxymethane (0.50 g, 4.75 mmol, 20.0 equiv.), azidotrimethylsilane (0.55 g, 4.75 mmol, 20.0 equiv.). The mixture was stirred overnight at room temperature, concentrated to yield N-(5-(2-(7′-(5-chloro-2-(1H-tetrazol-1-yl)phenyl)-5′-oxo-2′,3′-dihydro-5′H-spiro[cyclopropane-1,1′-indolizin]-3′-yl)-1H-imidazol-5-yl)-6-fluoropyridin-2-yl)acetamide as a yellow solid. LC/MS (ES, m/z): mass calculated for C₂₇H₂₁ClFN₉O₂: 557.15, measured: 558.15 [M+H]⁺.

### Step 15: (R*)-3′-(5-(6-Amino-2-fluoropyridin-3-yl)-1H-imidazol-2-yl)-7′-(5-chloro-2-(1H-tetrazol-1-yl)phenyl)-2′,3′-dihydro-5′H-spiro[cyclopropane-1,1′-indolizin]-5′-one

To a solution of N-(5-(2-(7′-(5-chloro-2-(1H-tetrazol-1-yl)phenyl)-5′-oxo-2′,3′-dihydro-5′H-spiro[cyclopropane-1,1′-indolizin]-3′-yl)-1H-imidazol-5-yl)-6-fluoropyridin-2-yl)acetamide (0.11 g, 0.19 mmol, 1.0 equiv.) in THE (5 mL) was add 2 M HCl (5 mL). The reaction mixture was stirred 1 h at 70° C., then concentrated under vacuum, purified by reverse column chromatography with CH₃CN/0.05% TFA water (5%→80%) to yield 3′-(5-(6-amino-2-fluoropyridin-3-yl)-1H-imidazol-2-yl)-7′-(5-chloro-2-(1H-tetrazol-1-yl)phenyl)-2′,3′-dihydro-5′H-spiro[cyclopropane-1,1′-indolizin]-5′-one as a yellow solid. The racemic mixture was purified by prep-chiral-HPLC with (Hex:DCM=3:1)(0.1% DEA):EtOH=50:50 to yield (S*)-3′-(5-(6-amino-2-fluoropyridin-3-yl)-1H-imidazol-2-yl)-7′-(5-chloro-2-(1H-tetrazol-1-yl)phenyl)-2′,3′-dihydro-5′H-spiro[cyclopropane-1,1′-indolizin]-5′-one as a white solid and ((R*)-3′-(5-(6-amino-2-fluoropyridin-3-yl)-1H-imidazol-2-yl)-7′-(5-chloro-2-(1H-tetrazol-1-yl)phenyl)-2′,3′-dihydro-5′H-spiro[cyclopropane-1,1′-indolizin]-5′-one as a white solid.

LC/MS (ES, m/z): mass calculated for C₂₅H₁₉ClFN₉O: 515.14, measured: 516.10 [M+H]⁺. ¹H NMR (400 MHz, DMSO-d₆) δ 11.93-12.16 (m, 1H), 9.66-9.68 (m, 1H), 7.98-8.04 (m, 1H), 7.85-7.75 (m, 3H), 7.09 (dd, J=4.0, 1.8 Hz, 1H), 6.38 (dd, J=8.2, 2.2 Hz, 1H), 6.23-6.27 (m, 2H), 5.91-5.98 (m, 1H), 5.67-5.72 (m, 1H), 5.43 (d, J=1.8 Hz, 1H), 2.69-2.79 (m, 1H), 2.20-2.36 (m, 1H), 1.14-1.21 (m, 1H), 1.05-1.13 (m, 1H), 0.97-1.04 (m, 1H), 0.89-0.95 (m, 1H). ¹⁹F NMR (376 MHz, DMSO-d₆) δ −70.86

### Example 4: (S)-3′-(5-(6-Amino-2-fluoropyridin-3-yl)-1H-imidazol-2-yl)-7′-(5-chloro-2-(1H-tetrazol-1-yl)phenyl)-2′,3′-dihydro-5′H-spiro[cyclopropane-1,1′-indolizin]-5′-one

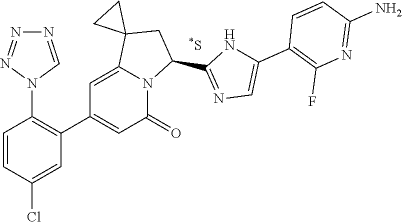 [SMILES: Nc1ccc(-c2cnc([C@@H]3CC4(CC4)c4cc(-c5cc(Cl)ccc5-n5cnnn5)cc(=O)n43)[nH]2)c(F)n1]

LC/MS: mass calculated for C₂₅H₁₉ClFN₉O: 515.14, measured (ES, m/z): 516.10 [M+H]⁺. ¹H NMR (400 MHz, DMSO-d₆) δ 9.66 (s, 1H), 7.88-7.96 (m, 1H), 7.75-7.86 (m, 3H), 7.25-7.43 (m, 1H), 6.47-6.62 (m, 2H), 6.42 (dd, J=8.2, 2.0 Hz, 1H), 5.98 (d, J=1.6 Hz, 1H), 5.71-5.81 (m, 1H), 5.50 (s, 1H), 2.65-2.79 (m, 1H), 2.29-2.41 (m, 1H), 1.08-1.17 (m, 1H), 0.90-1.02 (m, 2H), 0.79-0.89 (m, 1H). ¹⁹F NMR (376 MHz, DMSO-d₆) δ −69.92, −73.70.

### Example 5: (3'S)-7′-(3-Chloro-2-fluoro-6-(1H-tetrazol-1-yl)phenyl)-3′-(5-(3-fluoro-2-(1-hydroxyethyl)pyridin-4-yl)-1H-imidazol-2-yl)-2′,3′-dihydro-5′H-spiro[cyclopropane-1,1′-indolizin]-5′-one

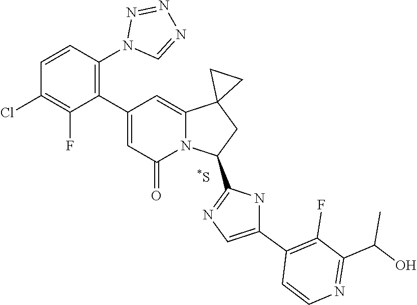 [SMILES: CC(O)c1nccc(-c2cnc([C@@H]3CC4(CC4)c4cc(-c5c(-n6cnnn6)ccc(Cl)c5F)cc(=O)n43)[nH]2)c1F]

LC/MS: mass calculated for C₂₇H₂₁ClF₂N₈O₂: 562.14, measured (ES, m/z): 563.10 [M+H]⁺. ¹H NMR (400 MHz, DMSO-d₆) δ 12.47 (s, 1H), 9.63 (d, J=0.9 Hz, 1H), 8.32 (d, J=5.0 Hz, 1H), 7.98 (t, J=8.4 Hz, 1H), 7.78-7.88 (m, 1H), 7.70 (dd, J=8.7, 1.5 Hz, 1H), 7.60 (d, J=3.9 Hz, 1H), 6.07 (s, 1H), 5.73 (dd, J=9.3, 2.8 Hz, 1H), 5.60 (d, J=1.6 Hz, 1H), 5.11-5.31 (m, 1H), 5.04 (q, J=6.5 Hz, 1H), 2.73-2.81 (m, 1H), 2.19-2.29 (m, 1H), 1.41 (d, J=6.5 Hz, 3H), 0.97-1.23 (m, 4H). ¹⁹F NMR (376 MHz, DMSO-d₆) δ −113.05, −130.80.

### Example 6: (3′R)-7′-(3-Chloro-2-fluoro-6-(1H-tetrazol-1-yl)phenyl)-3′-(5-(3-fluoro-2-(1-hydroxyethyl)pyridin-4-yl)-1H-imidazol-2-yl)-2′,3′-dihydro-5′H-spiro[cyclopropane-1,1′-indolizin]-5′-one

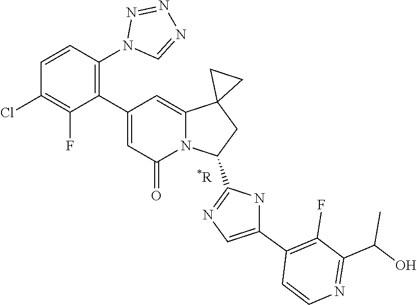 [SMILES: CC(O)c1nccc(-c2cnc([C@H]3CC4(CC4)c4cc(-c5c(-n6cnnn6)ccc(Cl)c5F)cc(=O)n43)[nH]2)c1F]

LC/MS: mass calculated for C₂₇H₂₁ClF₂N₈O₂:562.14, measured (ES, m/z): 563.10 [M+H]⁺. ¹H NMR (400 MHz, DMSO-d₆) δ 12.50 (s, 1H), 9.63 (d, J=1.0 Hz, 1H), 8.32 (d, J=5.0 Hz, 1H), 7.98 (t, J=8.4 Hz, 1H), 7.80-7.91 (m, 1H), 7.70 (dd, J=8.6, 1.5 Hz, 1H), 7.61 (d, J=3.9 Hz, 1H), 6.07 (s, 1H), 5.73 (dd, J=9.2, 2.9 Hz, 1H), 5.60 (d, J=1.6 Hz, 1H), 5.05 (q, J=6.5 Hz, 1H), 2.72-2.82 (m, 1H), 2.18-2.29 (m, 1H), 1.41 (d, J=6.5 Hz, 3H), 0.98-1.22 (m, 4H). ¹⁹F NMR (376 MHz, DMSO-d₆) δ −113.05, −130.61.

### Example 7: (S)-7′-(3-Chloro-2-fluoro-6-(1H-tetrazol-1-yl)phenyl)-3′-(5-(3-fluoro-2-(hydroxymethyl)pyridin-4-yl)-1H-imidazol-2-yl)-2′,3′-dihydro-5′H-spiro[cyclopropane-1,1′-indolizin]-5′-one

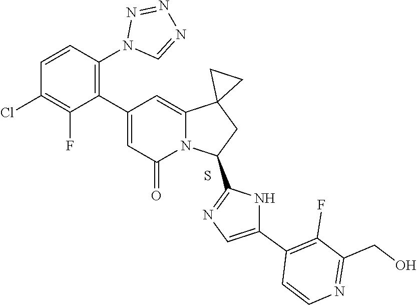

LC/MS: mass calculated for C₂₆H₁₉ClF₂N₈O₂: 548.13, measured (ES, m/z): 549.10 [M+H]⁺. ¹H NMR (400 MHz, DMSO-d₆) δ 12.48 (s, 1H), 9.63 (s, 1H), 8.31 (d, J=5.0 Hz, 1H), 7.98 (t, J=8.4 Hz, 1H), 7.84 (t, J=5.4 Hz, 1H), 7.71 (dd, J=8.7, 1.5 Hz, 1H), 7.59 (d, J=3.9 Hz, 1H), 6.07 (s, 1H), 5.73 (dd, J=9.2, 2.7 Hz, 1H), 5.60 (d, J=1.6 Hz, 1H), 5.20-5.36 (m, 1H), 4.60 (s, 2H), 2.71-2.82 (m, 1H), 2.21-2.30 (m, 1H), 0.94-1.22 (m, 4H). ¹⁹F NMR (376 MHz, DMSO-d₆) δ −73.41, −113.04, −130.25.

### Example 8: (R)-7′-(3-Chloro-2-fluoro-6-(1H-tetrazol-1-yl)phenyl)-3′-(5-(3-fluoro-2-(hydroxymethyl)pyridin-4-yl)-1H-imidazol-2-yl)-2′,3′-dihydro-5′H-spiro[cyclopropane-1,1′-indolizin]-5′-one

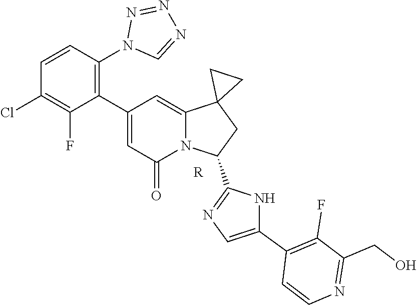

### Step 1: 2-(2-(((Tert-butyldimethylsilyl)oxy)methyl)-3-fluoropyridin-4-yl)-2-oxoethyl 7′-(6-amino-3-chloro-2-fluorophenyl)-5′-oxo-2′,3′-dihydro-5′H-spiro[cyclopropane-1,1′-indolizine]-3′-carboxylate

To a mixture of 7′-(6-amino-3-chloro-2-fluorophenyl)-5′-oxo-2′,3′-dihydro-5′H-spiro[cyclopropane-1,1′-indolizine]-3′-carboxylic acid (225 mg, 0.64 mmol, 1 equiv.) and K₂CO₃ (134 mg, 0.97 mmol, 1.5 equiv.) in acetonitrile (10 ml) was added 2-bromo-1-(2-(((tert-butyldimethylsilyl)oxy)methyl)-3-fluoropyridin-4-yl)ethan-1-one (350 mg, 0.97 mmol, 1.5 equiv.) and the mixture was stirred overnight at room temperature The mixture was diluted with H₂O and extracted with ethyl acetate twice. The combined organic layers were washed with brine, dried over anhydrous sodium sulfate and concentrated. The residue was purified by silica gel chromatography (0→5% methanol/dichloromethane) to yield 2-(2-(((tert-butyldimethylsilyl)oxy)methyl)-3-fluoropyridin-4-yl)-2-oxoethyl 7′-(6-amino-3-chloro-2-fluorophenyl)-5′-oxo-2′,3′-dihydro-5′H-spiro[cyclopropane-1,1′-indolizine]-3′-carboxylate as a yellow solid. LC/MS: mass calculated for C₃₁H₃₄ClF₂N₃O₅Si: 629.19, measured (ES, m/z): 630.15 [M+H]⁺.

### Step 2: 7′-(6-Amino-3-chloro-2-fluorophenyl)-3′-(5-(2-(((tert-butyldimethylsilyl)oxy)methyl)-3-fluoropyridin-4-yl)-1H-imidazol-2-yl)-2′,3′-dihydro-5′H-spiro[cyclopropane-1,1′-indolizin]-5′-one

To a solution of 2-(2-(((tert-butyldimethylsilyl)oxy)methyl)-3-fluoropyridin-4-yl)-2-oxoethyl 7′-(6-amino-3-chloro-2-fluorophenyl)-5′-oxo-2′,3′-dihydro-5′H-spiro[cyclopropane-1,1′-indolizine]-3′-carboxylate (0.24 g, 0.38 mmol, 1.0 equiv.) in toluene (5 ml) was added NH₄OAc (294 mg, 3.81 mmol, 10 equiv.) and AcOH (0.5 ml) and the solution was stirred for 2 h at 100° C. The solvent was evaporated under vacuum and the residue was purified by reverse phase chromatography on C18 (0→90% ACN/H₂O) to yield 7′-(6-amino-3-chloro-2-fluorophenyl)-3′-(5-(2-(((tert-butyldimethylsilyl)oxy)methyl)-3-fluoropyridin-4-yl)-1H-imidazol-2-yl)-2′,3′-dihydro-5′H-spiro[cyclopropane-1,1′-indolizin]-5′-one as a light yellow solid. LC/MS: mass calculated for C₃₁H₃₄ClF₂N₅O₂Si: 609.21, measured (ES, m/z): 610.35 [M+H]⁺.

### Step 3: 3′-(5-(2-(((Tert-butyldimethylsilyl)oxy)methyl)-3-fluoropyridin-4-yl)-1H-imidazol-2-yl)-7′-(3-chloro-2-fluoro-6-(1H-tetrazol-1-yl)phenyl)-2′,3′-dihydro-5′H-spiro[cyclopropane-1,1′-indolizin]-5′-one

A mixture of 7′-(6-amino-3-chloro-2-fluorophenyl)-3′-(5-(2-(((tert-butyldimethylsilyl)oxy)methyl)-3-fluoropyridin-4-yl)-1H-imidazol-2-yl)-2′,3′-dihydro-5′H-spiro[cyclopropane-1,1′-indolizin]-5′-one (140 mg, 0.23 mmol, 1.0 equiv.), trimethoxymethane (2 ml), azidotrimethylsilane (2 mL) and acetic acid (2 mL) was stirred 6 h at 60° C. The solvent was evaporated under vacuum to yield a mixture of 3′-(5-(2-(((tert-butyldimethylsilyl)oxy)methyl)-3-fluoropyridin-4-yl)-1H-imidazol-2-yl)-7′-(3-chloro-2-fluoro-6-(1H-tetrazol-1-yl)phenyl)-2′,3′-dihydro-5′H-spiro[cyclopropane-1,1′-indolizin]-5′-one and 7′-(3-chloro-2-fluoro-6-(1H-tetrazol-1-yl)phenyl)-3′-(5-(3-fluoro-2-(hydroxymethyl)pyridin-4-yl)-1H-imidazol-2-yl)-2′,3′-dihydro-5′H-spiro[cyclopropane-1,1′-indolizin]-5′-one. LC/MS: mass calculated for C₃₂H₃₃ClF₂N₈O₂Si: 662.22, measured (ES, m/z): 663.15 [M+H]⁺.

### Step 4: (R)-7′-(3-Chloro-2-fluoro-6-(1H-tetrazol-1-yl)phenyl)-3′-(5-(3-fluoro-2-(hydroxymethyl)pyridin-4-yl)-1H-imidazol-2-yl)-2′,3′-dihydro-5′H-spiro[cyclopropane-1,1′-indolizin]-5′-one

3′-(5-(2-(((Tert-butyldimethylsilyl)oxy)methyl)-3-fluoropyridin-4-yl)-1H-imidazol-2-yl)-7′-(3-chloro-2-fluoro-6-(1H-tetrazol-1-yl)phenyl)-2′,3′-dihydro-5′H-spiro[cyclopropane-1,1′-indolizin]-5′-one (0.1 g, 0.15 mmol, 1.0 equiv.) was added in HCl (5 mL, 4.0 M in 1,4-dioxane) and the solution stirred for 2 h at room temperature The solvent was evaporated under vacuum and the residue was purified by reverse phase chromatography on C18 (0→90% ACN/H₂O) to yield 7′-(3-chloro-2-fluoro-6-(1H-tetrazol-1-yl)phenyl)-3′-(5-(3-fluoro-2-(hydroxymethyl)pyridin-4-yl)-1H-imidazol-2-yl)-2′,3′-dihydro-5′H-spiro[cyclopropane-1,1′-indolizin]-5′-one. The racemic product was separated by chiral-HPLC to yield (R*)-7′-(3-chloro-2-fluoro-6-(1H-tetrazol-1-yl)phenyl)-3′-(5-(3-fluoro-2-(hydroxymethyl)pyridin-4-yl)-1H-imidazol-2-yl)-2′,3′-dihydro-5′H-spiro[cyclopropane-1,1′-indolizin]-5′-one as a light yellow solid.

LC/MS: mass calculated for C₂₆H₁₉ClF₂N₈O₂: 548.13, measured (ES, m/z): 549.10 [M+H]⁺. ¹H NMR (400 MHz, DMSO-d₆) δ 9.62 (s, 1H), 8.32 (d, J=5.1 Hz, 1H), 7.97 (dd, J=8.7, 7.7 Hz, 1H), 7.86 (t, J=5.4 Hz, 1H), 7.69 (dd, J=8.7, 1.5 Hz, 1H), 7.62 (d, J=3.9 Hz, 1H), 6.07 (d, J=1.6 Hz, 1H), 5.73 (dd, J=9.3, 2.8 Hz, 1H), 5.60 (d, J=1.6 Hz, 1H), 4.61 (d, J=2.2 Hz, 2H), 2.72-2.82 (m, 1H), 2.20-2.29 (m, 1H), 0.97-1.19 (m, 4H). ¹⁹F NMR (376 MHz, DMSO-d₆) δ −73.59, −113.07, −129.88.

### Example 9: 6-(2-((1*S,3*S)-7-(3-Chloro-2-fluoro-6-(1H-tetrazol-1-yl)phenyl)-1-methyl-5-oxo-1,2,3,5-tetrahydroindolizin-3-yl)-1H-imidazol-5-yl)-3,4-dihydro-1,7-naphthyridin-2(1H)-one

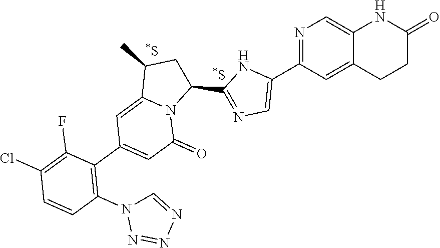 [SMILES: C[C@H]1C[C@@H](c2ncc(-c3cc4c(cn3)NC(=O)CC4)[nH]2)n2c1cc(-c1c(-n3cnnn3)ccc(Cl)c1F)cc2=O]

### Step 1: 2-Oxo-2-(2-oxo-1,2,3,4-tetrahydro-1,7-naphthyridin-6-yl)ethyl (1S,3S)-7-(6-amino-3-chloro-2-fluorophenyl)-1-methyl-5-oxo-1,2,3,5-tetrahydroindolizine-3-carboxylate

To a solution of (1S,3S)-7-(6-amino-3-chloro-2-fluorophenyl)-1-methyl-5-oxo-1,2,3,5-tetrahydroindolizine-3-carboxylic acid (120 mg, 0.35 mmol) in DMF (10 mL) was added K₂CO₃ (73 mg, 0.53 mmol). After 0.5 h of stirring, 6-(2-bromoacetyl)-3,4-dihydro-1,7-naphthyridin-2(1H)-one (14 mg, 0.53 mmol) was added. The mixture was stirred at room temperature for 1 h. After partitioning with ethyl acetate and water, the organic phase was washed with brine, dried over Na₂SO₄, filtered and concentrated. The residue was purified by reverse phase chromatography on C18 column with CH₃CN/0.05% TFA water (5%-40%) to yield the 2-oxo-2-(2-oxo-1,2,3,4-tetrahydro-1,7-naphthyridin-6-yl)ethyl (1S,3S)-7-(6-amino-3-chloro-2-fluorophenyl)-1-methyl-5-oxo-1,2,3,5-tetrahydroindolizine-3-carboxylate as a yellow solid.

### Step 2: 6-(2-((1S,3S)-7-(6-amino-3-chloro-2-fluorophenyl)-1-methyl-5-oxo-1,2,3,5-tetrahydroindolizin-3-yl)-1H-imidazol-5-yl)-3,4-dihydro-1,7-naphthyridin-2(1H)-one

To a solution of 2-oxo-2-(2-oxo-1,2,3,4-tetrahydro-1,7-naphthyridin-6-yl)ethyl (1S,3S)-7-(6-amino-3-chloro-2-fluorophenyl)-1-methyl-5-oxo-1,2,3,5-tetrahydroindolizine-3-carboxylate (200 mg, 0.38 mmol) in toluene (10 mL) and acetic acid (0.5 mL) was added ammonium acetate (587 mg, 7.62 mmol). The mixture stirred at 100° C. for 1 h and the solvent was roto-evaporated under vacuum. The residue was purified by silica gel chromatography (0-20% MeOH/DCM) to yield 6-(2-((1S,3S)-7-(6-amino-3-chloro-2-fluorophenyl)-1-methyl-5-oxo-1,2,3,5-tetrahydroindolizin-3-yl)-1H-imidazol-5-yl)-3,4-dihydro-1,7-naphthyridin-2(1H)-one as a yellow solid.

### Step 3: 6-(2-((1S,3S)-7-(3-chloro-2-fluoro-6-(1H-tetrazol-1-yl)phenyl)-1-methyl-5-oxo-1,2,3,5-tetrahydroindolizin-3-yl)-1H-imidazol-5-yl)-3,4-dihydro-1,7-naphthyridin-2(1H)-one

To a solution of 6-(2-((1S,3S)-7-(6-amino-3-chloro-2-fluorophenyl)-1-methyl-5-oxo-1,2,3,5-tetrahydroindolizin-3-yl)-1H-imidazol-5-yl)-3,4-dihydro-1,7-naphthyridin-2(1H)-one (20 mg, 0.040 mmol) in trimethoxymethane (1 mL) were added azidotrimethylsilane(1 mL) and AcOH (2 mL). The mixture was stirred at room temperature overnight. The mixture was then concentrated and the residue was purified by Prep-HPLC with the following conditions (1 #-Waters 2767-5): Column: XBridge Shield RP18 OBD Column, 30*150 mm, 5 um; Mobile Phase A: Water (10 mmol/L NH₄HCO₃), Mobile Phase B: ACN; Flow rate: 60 mL/min; Gradient: 14 B to 41 B in 8 min; 220 nm; room temperature 1:7.3; room temperature; After lyophilization, 6-(2-((1 S*,3S*)-7-(3-chloro-2-fluoro-6-(1H-tetrazol-1-yl) phenyl)-1-methyl-5-oxo-1,2,3,5-tetrahydroindolizin-3-yl)-1H-imidazol-5-yl)-3,4-dihydro-1,7-naphthyridin-2(1H)-one was isolated as off-white solid.

LC/MS: mass calculated for C₂₇H₂₁ClFN₉O₂: 557.15, measured (ES, m/z): 558.25 [M+H]⁺. ¹H NMR (400 MHz, Methanol-d₄) δ 9.37 (s, 1H), 8.05 (s, 1H), 7.84-7.96 (m, 1H), 7.65-7.80 (m, 1H), 7.22-7.63 (m, 2H), 6.09-6.35 (m, 2H), 5.55-5.69 (m, 1H), 4.49-4.61 (m, 1H), 3.39-3.50 (m, 1H), 3.01-3.18 (m, 2H), 2.75-2.91 (m, 1H), 2.55-2.69 (m, 2H), 2.10-2.28 (m, 1H), 1.40-1.48 (m, 3H). ¹⁹F NMR (376 MHz, Methanol-d₄) δ −113.79.

### Example 10: (1 S*,3S*)-3-(5-(6-Aminopyridazin-3-yl)-1H-imidazol-2-yl)-7-(3-chloro-2-fluoro-6-(1H-tetrazol-1-yl)phenyl)-1-methyl-2,3-dihydroindolizin-5(1H)-one

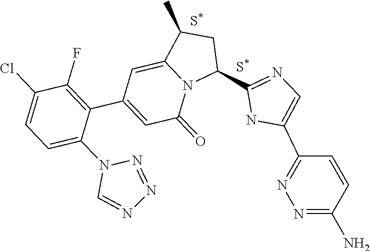 [SMILES: C[C@H]1C[C@@H](c2ncc(-c3ccc(N)nn3)[nH]2)n2c1cc(-c1c(-n3cnnn3)ccc(Cl)c1F)cc2=O]

### Step 1: 2-(6-acetamidopyridazin-3-yl)-2-oxoethyl 7-(6-amino-3-chloro-2-fluorophenyl)-1-methyl-5-oxo-1,2,3,5-tetrahydroindolizine-3-carboxylate

To a solution of 7-(6-amino-3-chloro-2-fluorophenyl)-1-methyl-5-oxo-1,2,3,5-tetrahydroindolizine-3-carboxylic acid (100 mg, 0.297 mmol, 1 equiv) in acetonitrile (3 mL) was added Cs₂CO₃ (126 mg, 0.39 mmol, 1 equiv). After the reaction mixture was stirred at room temperature 30 min, the N-(6-(2-bromoacetyl)pyridazin-3-yl)acetamide (115 mg, 0.445 mmol, 1.5 equiv) was added. The mixture was stirred at room temperature for 1 h. The mixture was concentrated and the residue was purified by silica gel chromatography (0→10% MeOH/DCM) to yield 2-(6-acetamidopyridazin-3-yl)-2-oxoethyl 7-(6-amino-3-chloro-2-fluorophenyl)-1-methyl-5-oxo-1,2,3,5-tetrahydroindolizine-3-carboxylate as an off-white solid. LC/MS: mass calculated for C₂₄H₂₁ClFN₅O₅: 513.12, measured: 514.10 [M+H]+.

### Step 2: N-(6-(2-(7-(6-amino-3-chloro-2-fluorophenyl)-1-methyl-5-oxo-1,2,3,5-tetrahydroindolizin-3-yl)-1H-imidazol-5-yl)pyridazin-3-yl)acetamide

To a solution of 2-(6-acetamidopyridazin-3-yl)-2-oxoethyl 7-(6-amino-3-chloro-2-fluorophenyl)-1-methyl-5-oxo-1,2,3,5-tetrahydroindolizine-3-carboxylate (140 mg, 0.27 mmol, 1 eq) in toluene (20 mL) were added ammonium acetate (315 mg, 4.08 mmol, 15 eq) and glacial acetic acid (164 mg, 2.72 mmol, 10 eq). The reaction mixture was stirred at 100° C. for 1 h. The mixture was concentrated, and purified by silica gel chromatography (0→10% MeOH/DCM) to yield N-(6-(2-(7-(6-amino-3-chloro-2-fluorophenyl)-1-methyl-5-oxo-1,2,3,5-tetrahydroindolizin-3-yl)-1H-imidazol-5-yl)pyridazin-3-yl)acetamide as a light yellow solid. LC/MS: mass calculated for C₂₄H₂₁ClFN₇O₂: 493.14, measured: 494.20 [M+H]⁺.

### Step 3: N-(6-(2-(7-(3-chloro-2-fluoro-6-(1H-tetrazol-1-yl)phenyl)-1-methyl-5-oxo-1,2,3,5-tetrahydroindolizin-3-yl)-1H-imidazol-5-yl)pyridazin-3-yl)acetamide

To a solution of N-(6-(2-(7-(6-amino-3-chloro-2-fluorophenyl)-1-methyl-5-oxo-1,2,3,5-tetrahydroindolizin-3-yl)-1H-imidazol-5-yl)pyridazin-3-yl)acetamide (80 mg, 0.16 mmol, 1 eq) in acetic acid (15 mL) were added trimethoxymethane (172 mg, 1.62 mmol, 10 eq), and azidotrimethylsilane (187 mg, 1.62 mmol, 10 eq). The resulting mixture was stirred overnight at room temperature. Then the solvent was concentrated under vacuum. The residue was purified by reverse column chromatography with CH₃CN/0.05% TFA water (5%→80%) to N-(6-(2-(7-(3-chloro-2-fluoro-6-(1H-tetrazol-1-yl)phenyl)-1-methyl-5-oxo-1,2,3,5-tetrahydroindolizin-3-yl)-1H-imidazol-5-yl)pyridazin-3-yl)acetamide as an off-white solid. LC/MS: mass calculated for C₂₅H₂₀ClFN₁₀O₂: 546.14, measured: 547.20 [M+H]⁺.

### Step 4: (1S,3S)-3-(5-(6-aminopyridazin-3-yl)-1H-imidazol-2-yl)-7-(3-chloro-2-fluoro-6-(1H-tetrazol-1-yl)phenyl)-1-methyl-2,3-dihydroindolizin-5(1H)-one

To a solution of N-(6-(2-((1S,3S)-7-(3-chloro-2-fluoro-6-(1H-tetrazol-1-yl)phenyl)-1-methyl-5-oxo-1,2,3,5-tetrahydroindolizin-3-yl)-1H-imidazol-5-yl)pyridazin-3-yl)acetamide (50 mg, 0.091 mmol, 1 eq) in tetrahydrofuran (5 mL) was hydrochloric acid (5 mL, 2N). The reaction mixture was stirred at 60° C. for 4 h. The mixture was concentrated under vacuum. The resident was purified by reverse column chromatography with CH₃CN/0.05% TFA water (5%→80%) to yield (1S,3S)-3-(5-(2-aminopyrimidin-4-yl)-1H-imidazol-2-yl)-7-(3-chloro-2-fluoro-6-(1H-tetrazol-1-yl)phenyl)-1-methyl-2,3-dihydroindolizin-5(1H)-one as an off-white solid.

LC/MS: mass calculated for C₂₃H₁₈ClFN₁₀O: 504.13, measured (ES, m/z): 505.20 [M+H]⁺. ¹H NMR (400 MHz, DMSO-d₆) δ 9.67 (s, 1H), 8.09-8.26 (m, 1H), 8.00-8.06 (m, 1H), 7.90 (s, 1H), 7.70-7.80 (m, 1H), 7.42 (d, J=9.5 Hz, 1H), 6.10 (s, 2H), 5.50-5.62 (m, 1H), 3.30-3.45 (m, 1H), 2.75-2.85 (m, 1H), 1.19-2.03 (m, 1H), 1.30 (d, J=7.0 Hz, 3H). ¹⁹F NMR (376 MHz, DMSO-d₆) −74.14, 113.02.

### Example 11: N-(4-(2-((1S,3S)-7-(3-chloro-2-fluoro-6-(1H-tetrazol-1-yl)phenyl)-1-methyl-5-oxo-1,2,3,5-tetrahydroindolizin-3-yl)-1H-imidazol-5-yl)phenyl)methanesulfonamide

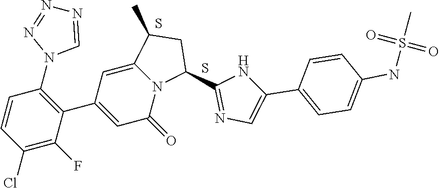

### Step 1: 2-(4-(methylsulfonamido)phenyl)-2-oxoethyl (1 S,3S)-7-(6-amino-3-chloro-2-fluorophenyl)-1-methyl-5-oxo-1,2,3,5-tetrahydroindolizine-3-carboxylate

To a solution of rac-(6R,8R)-2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylic acid (120 mg, 0.35 mmol, 1 equiv.) and in DMF (1 mL) was added K₂CO₃ (148 mg, 1.06 mmol, 3 equiv.) under N₂. The reaction mixture was stirred for 0.5 h, then a solution of N-(5-(2-bromoacetyl) pyridin-2-yl)cyclopropanecarboxamide (106 mg, 0.42 mmol, 1.3 equiv.) was added. The reaction mixture was stirred 1 h at room temperature, then concentrated under vacuum. The mixture was purified by reverse-phase chromatography (C18, 330 g, CH₃CN/H₂O (0.05% TFA)=10%-70%) to yield rac-2-(4-(methylsulfonamido)phenyl)-2-oxoethyl (1S,3S)-7-(6-amino-3-chloro-2-fluorophenyl)-1-methyl-5-oxo-1,2,3,5-tetrahydroindolizine-3-carboxylate as a yellow oil.

### Step 2: N-(4-(2-((1S,3S)-7-(6-amino-3-chloro-2-fluorophenyl)-1-methyl-5-oxo-1,2,3,5-tetrahydroindolizin-3-yl)-1H-imidazol-5-yl)phenyl)methanesulfonamide

To a solution of rac-2-(4-(methylsulfonamido)phenyl)-2-oxoethyl (1R,3R)-7-(6-amino-3-chloro-2-fluorophenyl)-1-methyl-5-oxo-1,2,3,5-tetrahydroindolizine-3-carboxylate (80 mg, 0.14 mmol, 1 equiv.) and ammonium acetate (225 mg, 2.92 mol, 20 equiv.) in toluene (4 mL) was added AcOH (1 ml). The reaction mixture was stirred 2 h at 90° C., and then cooled to room temperature. The reaction was quenched with water, the resulting mixture was extracted with EA, washed with brine, dried over Na₂SO₄ and concentrated under vacuum. The residue was purified by silica gel chromatography with MeOH/DCM (0→10%) to yield N-(4-(2-((1S,3S)-7-(6-amino-3-chloro-2-fluorophenyl)-1-methyl-5-oxo-1,2,3,5-tetrahydroindolizin-3-yl)-1H-imidazol-5-yl)phenyl)methanesulfonamide as yellow oil.

### Step 3: N-(4-(2-((1S,3S)-7-(3-chloro-2-fluoro-6-(1H-tetrazol-1-yl)phenyl)-1-methyl-5-oxo-1,2,3,5-tetrahydroindolizin-3-yl)-1H-imidazol-5-yl)phenyl)methanesulfonamide

To a solution of (1S,3S)-7-(6-amino-3-chloro-2-fluorophenyl)-1-methyl-3-(5-(4-((methylsulfonyl)-12-azaneyl)phenyl)-1H-imidazol-2-yl)-2,3-dihydroindolizin-5(1H)-one (50 mg, 0.095 mmol, 1.0 equiv) and azidotrimethylsilane (163 mg, 1.41 mmol, 15 equiv.) in CH₃COOH (2 mL) was added trimethoxymethane (150 mg, 1.41 mmol, 15 equiv.) under N₂. The reaction mixture was stirred overnight at room temperature, then concentrated under vacuum. The mixture was purified by reverse-phase chromatography (C18, 330 g, CH₃CN/H₂O (0.05% TFA)=10%→70%) to yield N-(4-(2-((1S,3S)-7-(3-chloro-2-fluoro-6-(1H-tetrazol-1-yl)phenyl)-1-methyl-5-oxo-1,2,3,5-tetrahydroindolizin-3-yl)-1H-imidazol-5-yl)phenyl)methanesulfonamide.

LC/MS: mass calculated for C₂₆H₂₂ClFN₈O₃S: 580.12, measured (ES, m/z): 581.25 [M+H]⁺. ¹H NMR (300 MHz, Methanol-d₄) δ 9.40 (s, 1H), 7.85-7.95 (m, 1H), 7.64-7.73 (m, 2H), 7.55-7.65 (m, 1H), 7.48 (s, 1H), 7.26-7.36 (m, 2H), 6.22 (d, J=11.1 Hz, 2H), 5.65 (t, J=8.2 Hz, 1H), 3.39-3.53 (m, 1H), 3.00 (s, 3H), 2.84-3.00 (m, 1H), 2.08-2.25 (m, 1H), 1.43 (d, J=6.9 Hz, 3H). ¹⁹F NMR (282 MHz, Methanol-d₄) δ −113.74.

### Example 12: 5-(2-((1S,3S)-7-(3-Chloro-2-fluoro-6-(1H-tetrazol-1-yl)phenyl)-1-methyl-5-oxo-1,2,3,5-tetrahydroindolizin-3-yl)-1H-imidazol-5-yl)-N-methylpicolinamide

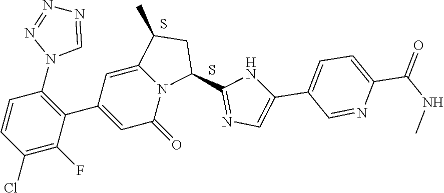

LC/MS: mass calculated for C₂₆H₂₁ClFN₉O₂: 545.15, measured (ES, m/z): 546.25 [M+H]⁺. ¹H NMR (400 MHz, DMSO-d₆) δ 12.47 (s, 1H), 9.66 (s, 1H), 8.96 (d, J=2.1 Hz, 1H), 8.65-8.80 (m, 1H), 8.15-8.25 (m, 1H), 7.96-8.08 (m, 2H), 7.83 (s, 1H), 7.72-7.78 (m, 1H), 6.11 (s, 1H), 6.03 (s, 1H), 5.50-5.60 (m, 1H), 2.71-2.85 (m, 5H), 1.95-2.05 (m, 1H), 1.34 (d, J=7.1 Hz, 3H). ¹⁹F NMR (376 MHz, DMSO-d₆) δ −113.02.

### Example 13: 4-(2-((1S,3S)-7-(3-Chloro-2-fluoro-6-(1H-tetrazol-1-yl)phenyl)-1-methyl-5-oxo-1,2,3,5-tetrahydroindolizin-3-yl)-1H-imidazol-5-yl)-N-methylpicolinamide

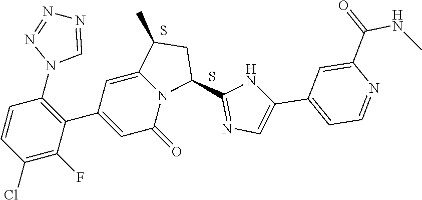

LC/MS: mass calculated for C₂₆H₂₁ClFN₉O₂: 545.15, measured (ES, m/z): 546.30 [M+H]⁺. ¹H NMR (400 MHz, DMSO-d₆) δ 12.54 (s, 1H), 9.67 (s, 1H), 8.75 (d, J=4.9 Hz, 1H), 8.50-8.61 (m, 1H), 8.29 (d, J=1.7 Hz, 1H), 7.98-8.06 (m, 2H), 7.82-7.88 (m, 1H), 7.70-7.80 (m, 1H), 6.08 (d, J=20.3 Hz, 2H), 5.45-5.66 (m, 1H), 2.71-2.87 (m, 5H), 2.00-2.05 (m, 1H), 1.34 (d, J=7.1 Hz, 3H). ¹⁹F NMR (376 MHz, DMSO-d₆) δ −112.92.

### Example 14: 5-(2-((1S,3S)-7-(3-Chloro-2-fluoro-6-(1H-tetrazol-1-yl)phenyl)-1-methyl-5-oxo-1,2,3,5-tetrahydroindolizin-3-yl)-1H-imidazol-5-yl)-1-methyl-1H-pyrazole-4-carbonitrile

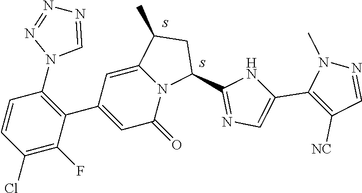

LC/MS: mass calculated for C₂₄H₁₈ClFN₁₀O: 516.13, measured (ES, m/z): 517.25 [M+H]⁺. ¹H NMR (400 MHz, DMSO-d₆) δ 12.81 (brs., 1H), 9.65 (s, 1H), 8.08-7.95 (m, 2H), 7.70-7.76 (m, 1H), 7.68 (s, 1H), 6.14 (s, 1H), 6.02 (s, 1H), 5.60-5.65 (m, 1H), 4.12 (s, 3H), 3.30-3.45 (m, 1H), 2.75-2.90 (m, 1H), 1.95-2.05 (m, 1H), 1.29 (d, J=7.1 Hz, 3H). ¹⁹F NMR (376 MHz, DMSO-d₆) −74.65, −113.25.

### Example 15: (1S,3S)-3-(5-(3-Amino-1-methyl-1H-pyrazol-5-yl)-1H-imidazol-2-yl)-7-(3-chloro-2-fluoro-6-(1H-tetrazol-1-yl)phenyl)-1-methyl-2,3-dihydroindolizin-5(1H)-one

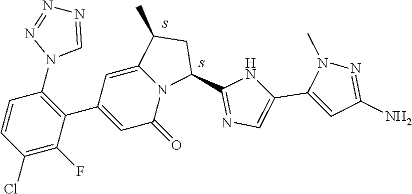

### Step 1: 2-(3-acetamido-1-methyl-1H-pyrazol-5-yl)-2-oxoethyl (1S,3S)-7-(6-amino-3-chloro-2-fluorophenyl)-1-methyl-5-oxo-1,2,3,5-tetrahydroindolizine-3-carboxylate

To a solution of (1S,3S)-7-(6-amino-3-chloro-2-fluorophenyl)-1-methyl-5-oxo-1,2,3,5-tetrahydroindolizine-3-carboxylic acid (100 mg, 0.30 mmol, 1 equiv) in DMF (2 mL) was added potassium carbonate (120 mg, 0.89 mmol, 3 equiv).

After the reaction mixture was stirred at room temperature 30 min, N-(4-(2-bromoacetyl)pyrimidin-2-yl)acetamide (150 mg, 0.59 mmol, 2 equiv) was added. The mixture was stirred at room temperature for 2 h. The mixture was concentrated, and the resulting residue was purified by silica gel chromatography (0→10% MeOH/DCM) to yield 2-(3-acetamido-1-methyl-1H-pyrazol-5-yl)-2-oxoethyl (1S,3S)-7-(6-amino-3-chloro-2-fluorophenyl)-1-methyl-5-oxo-1,2,3,5-tetrahydroindolizine-3-carboxylate as a light yellow solid. LC/MS: mass calculated for C₂₄H₂₃ClFN₅O₅: 515.14, measured: 516.15 [M+H]⁺.

### Step 2: N-(5-(2-((1S,3S)-7-(6-amino-3-chloro-2-fluorophenyl)-1-methyl-5-oxo-1,2,3,5-tetrahydroindolizin-3-yl)-1H-imidazol-5-yl)-1-methyl-1H-pyrazol-3-yl)acetamide

To a solution of 2-(3-acetamido-1-methyl-1H-pyrazol-5-yl)-2-oxoethyl (1S,3S)-7-(6-amino-3-chloro-2-fluorophenyl)-1-methyl-5-oxo-1,2,3,5-tetrahydroindolizine-3-carboxylate (90 mg, 0.17 mmol, 1 eq) in toluene (20 mL) were added ammonium acetate (202 mg, 2.61 mmol, 15 eq.) and acetic acid (105 mg, 1.744 mmol, 10 eq.). The reaction mixture was stirred at 100° C. for 2 h. The mixture was concentrated, and purified by silica gel chromatography (0-10% MeOH/DCM) to yield N-(5-(2-((1S,3S)-7-(6-amino-3-chloro-2-fluorophenyl)-1-methyl-5-oxo-1,2,3,5-tetrahydroindolizin-3-yl)-1H-imidazol-5-yl)-1-methyl-1H-pyrazol-3-yl)acetamide as light yellow solid. LC/MS: mass calculated for C₂₄H₂₃ClFN₇O₂: 495.16, measured: 496.15 [M+H]⁺.

### Step 3: N-(5-(2-((1S,3S)-7-(3-chloro-2-fluoro-6-(1H-tetrazol-1-yl)phenyl)-1-methyl-5-oxo-1,2,3,5-tetrahydroindolizin-3-yl)-1H-imidazol-5-yl)-1-methyl-1H-pyrazol-3-yl)acetamide

To a solution of N-(5-(2-((1S,3S)-7-(6-amino-3-chloro-2-fluorophenyl)-1-methyl-5-oxo-1,2,3,5-tetrahydroindolizin-3-yl)-1H-imidazol-5-yl)-1-methyl-1H-pyrazol-3-yl)acetamide (50 mg, 0.101 mmol, 1 eq.) in acetic acid (15 mL) were added trimethoxymethane (106 mg, 1.00 mmol, 10 eq.) and azidotrimethylsilane (116 mg, 1.00 mmol, 10 eq.). The resulting mixture was stirred overnight at room temperature, then concentrated under vacuum to yield N-(5-(2-((1S,3S)-7-(3-chloro-2-fluoro-6-(1H-tetrazol-1-yl)phenyl)-1-methyl-5-oxo-1,2,3,5-tetrahydroindolizin-3-yl)-1H-imidazol-5-yl)-1-methyl-1H-pyrazol-3-yl)acetamide as a light yellow solid, which was used in the next step without further purification. LC/MS: mass calculated for C₂₅H₂₂ClFN₁₀O₂: 548.16, measured: 549.20 [M+H]+.

### Step 4: (1S,3S)-3-(5-(3-amino-1-methyl-1H-pyrazol-5-yl)-1H-imidazol-2-yl)-7-(3-chloro-2-fluoro-6-(1H-tetrazol-1-yl)phenyl)-1-methyl-2,3-dihydroindolizin-5(1H)-one

To a solution of N-(5-(2-((1S,3S)-7-(3-chloro-2-fluoro-6-(1H-tetrazol-1-yl)phenyl)-1-methyl-5-oxo-1,2,3,5-tetrahydroindolizin-3-yl)-1H-imidazol-5-yl)-1-methyl-1H-pyrazol-3-yl)acetamide (50 mg, 0.091 mmol, 1 eq) in tetrahydrofuran (5 mL) was added hydrochloric acid (5 mL, 2N). The reaction mixture was stirred at 60° C. for 4 h. The mixture was concentrated under vacuum. The residue was purified by reverse column chromatography with CH₃CN/0.05% TFA water (5%→80%) to yield (1S,3S)-3-(5-(3-amino-1-methyl-1H-pyrazol-5-yl)-1H-imidazol-2-yl)-7-(3-chloro-2-fluoro-6-(1H-tetrazol-1-yl)phenyl)-1-methyl-2,3-dihydroindolizin-5(1H)-one as an off-white solid.

LC/MS: mass calculated for C₂₃H₂₀ClFN₁₀O: 506.15, measured (ES, m/z): 507.25 [M+H]+. ¹H NMR (400 MHz, DMSO-d₆) δ 12.24 (brs., 1H), 9.65 (s, 1H), 8.00-8.05 (m, 1H), 7.70-7.76 (m, 1H), 7.29 (s, 1H), 6.11 (s, 1H), 6.00 (s, 1H), 5.60 (s, 1H), 5.50-5.58 (m, 1H), 3.73 (s, 3H), 3.25-3.40 (m, 1H), 2.72-2.82 (m, 1H), 1.95-2.02 (m, 1H), 1.30 (d, J=7.1 Hz, 3H). ¹⁹F NMR (376 MHz, DMSO-d₆) −73.42, −113.17.

### Example 16: (1S,3S)-7-(3-Chloro-2-fluoro-6-(1H-tetrazol-1-yl)phenyl)-1-methyl-3-(5-(6-oxo-1,6-dihydropyridin-3-yl)-1H-imidazol-2-yl)-2,3-dihydroindolizin-5(1H)-one

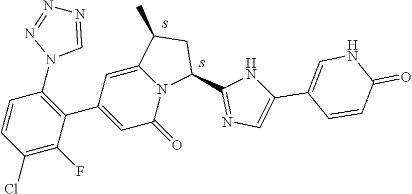

LC/MS: mass calculated for C₂₄H₁₈ClFN₈O₂: 504.12, measured (ES, m/z): 505.25 [M+H]⁺. H NMR (400 MHz, DMSO-d₆) δ 12.08 (s, 1H), 11.54 (brs., 1H), 9.65 (s, 1H), 7.95-8.05 (m, 1H), 7.70-7.85 (m, 2H), 7.59 (s, 1H), 7.33 (s, 1H), 6.36 (d, J=9.4 Hz, 1H), 6.09 (s, 1H), 6.01 (s, 1H), 5.40-5.55 (m, 1H), 2.72-2.80 (m, 2H), 1.93-2.00 (m, 1H), 1.33 (d, J=7.1 Hz, 3H). ¹⁹F NMR (376 MHz, DMSO-d₆) δ −112.96, −113.02.

### Example 17: (R)-3′-(5-(2-Aminothiazol-5-yl)-1H-imidazol-2-yl)-7′-(3-chloro-2-fluoro-6-(1H-tetrazol-1-yl)phenyl)-2′,3′-dihydro-5′H-spiro[cyclopropane-1,1′-indolizin]-5′-one

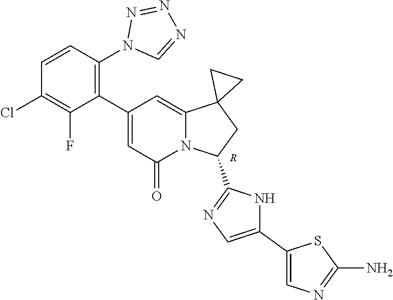

LC/MS: mass calculated for C₂₃H₁₇ClFN₉OS: 521.09, measured (ES, m/z): 522.05 [M+H]⁺. ¹H NMR (300 MHz, DMSO-d₆): δ 11.92 (s, 1H), 9.64 (d, J=3.6 Hz, 1H), 7.95-8.05 (m, 1H), 7.72 (dd, J=8.7, 1.5 Hz, 1H), 7.01-7.15 (m, 2H), 6.80 (s, 2H), 6.05 (d, J=11.6 Hz, 1H), 5.52-5.73 (m, 2H), 2.65-2.79 (m, 1H), 2.23-2.35 (m, 1H), 1.07-1.27 (m, 2H), 0.95-1.05 (m, 2H). ¹⁹F NMR (282 MHz, DMSO-d₆): δ −112.90.

### Example 18: (S)-3′-(5-(2-Aminothiazol-5-yl)-1H-imidazol-2-yl)-7′-(3-chloro-2-fluoro-6-(1H-tetrazol-1-yl)phenyl)-2′,3′-dihydro-5′H-spiro[cyclopropane-1,1′-indolizin]-5′-one

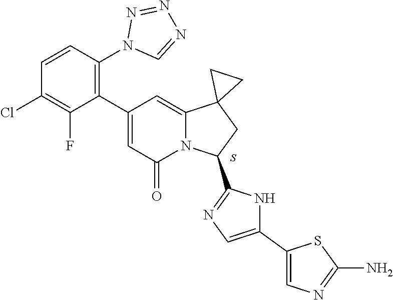

LC/MS: mass calculated for C₂₃H₁₇ClFN₉OS: 521.09, measured (ES, m/z): 522.05 [M+H]⁺. ¹H NMR (300 MHz, DMSO-d₆): δ 11.92 (s, 1H), 9.64 (d, J=3.6 Hz, 1H), 7.95-8.05 (m, 1H), 7.70-7.75 (m, 1H), 7.02-7.15 (m, 2H), 6.80 (s, 2H), 6.07 (d, J=1.6 Hz, 1H), 5.54-5.70 (m, 2H), 2.65-2.83 (m, 1H), 2.23-2.35 (m, 1H), 1.06-1.29 (m, 2H), 0.85-1.05 (m, 2H). ¹⁹F NMR (282 MHz, DMSO-d₆): δ −73.39, −112.90.

### Example 19: (1S,3S)-3-(5-(2-Amino-5-fluoropyridin-4-yl)-1H-imidazol-2-yl)-7-(3-chloro-2-fluoro-6-(1H-tetrazol-1-yl)phenyl)-1-methyl-2,3-dihydroindolizin-5(1H)-one

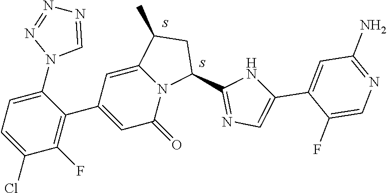

LC/MS: mass calculated for C₂₄H₁₈ClF₂N₉O: 521.13, measured (ES, m/z): 522.20 [M+H]⁺. ¹H NMR (400 MHz, DMSO-d₆) δ 12.49 (s, 1H), 9.69 (s, 1H), 7.98-8.04 (m, 1H), 7.83 (d, J=3.1 Hz, 1H), 7.71-7.79 (m, 1H), 7.44-7.49 (m, 1H), 6.99 (d, J=5.4 Hz, 1H), 6.08 (d, J=14.1 Hz, 2H), 5.79 (s, 2H), 5.50-5.55 (m, 1H), 2.68-2.85 (m, 1H), 1.95-2.02 (m, 2H), 1.31 (d, J=7.1 Hz, 3H). ¹⁹F NMR (376 MHz, DMSO-d₆) δ −112.97, −148.08.

### Example 20: (1S,3S)-3-(5-(2-Aminopyrimidin-4-yl)-1H-imidazol-2-yl)-7-(3-chloro-2-fluoro-6-(1H-tetrazol-1-yl)phenyl)-1-methyl-2,3-dihydroindolizin-5(1H)-one

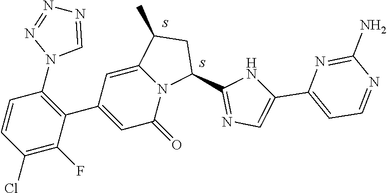

LC/MS: mass calculated for C₂₃H₁₈ClFN₁₀O: 504.13, measured (ES, m/z): 505.20 [M+H]⁺. ¹H NMR (400 MHz, DMSO-d₆) δ 12.15 (s, 1H), 9.65 (s, 1H), 8.50-8.60 (m, 2H), 7.95-8.05 (m, 1H), 7.71-7.76 (m, 1H), 7.41 (d, J=2.0 Hz, 1H), 6.55 (s, 2H), 6.08 (d, J=13.2 Hz, 1H), 6.01 (s, 1H), 5.45-5.65 (m, 1H), 2.70-2.80 (m, 2H), 1.98-2.03 (m, 1H), 1.33 (d, J=7.2 Hz, 3H). ¹⁹F NMR (376 MHz, DMSO-d₆) −113.08.

### Example 21: (1S,3S)-3-(5-(5-Amino-6-fluoropyridin-3-yl)-1H-imidazol-2-yl)-7-(3-chloro-2-fluoro-6-(1H-tetrazol-1-yl)phenyl)-1-methyl-2,3-dihydroindolizin-5(1H)-one

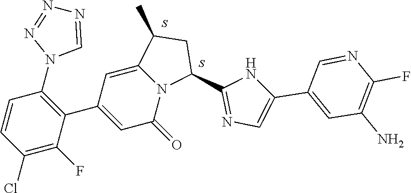

LC/MS: mass calculated for C₂₄H₁₈ClF₂N₉O: 521.13, measured (ES, m/z): 522.25 [M+H]⁺. ¹H NMR (400 MHz, DMSO-d₆) δ 9.67 (s, 1H), 8.00-8.06 (m, 1H), 7.78-7.80 (m, 1H), 7.72-7.76 (m, 1H), 7.67-7.70 (m, 1H), 7.41 (dd, J=10.3, 2.2 Hz, 1H), 6.09-6.19 (m, 2H), 5.61 (t, J=8.2 Hz, 1H), 3.29-3.44 (m, 1H), 2.75-2.90 (m, 1H), 1.90-2.01 (m, 1H), 1.32 (d, J=6.9 Hz, 3H). ¹⁹F NMR (376 MHz, DMSO-d₆) −74.12, −112.80.

### Example 22: (R*)-3′-(5-(2-Aminothiazol-5-yl)-1H-imidazol-2-yl)-7′-(3-chloro-2-fluoro-6-(1H-tetrazol-1-yl)phenyl)-2′,3′-dihydro-5′H-spiro[cyclopropane-1,1′-indolizin]-5′-one

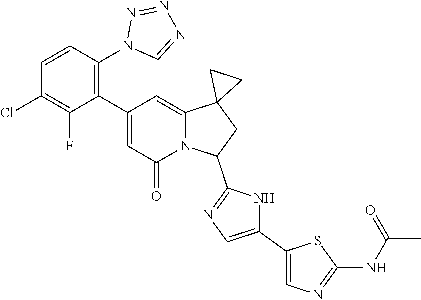 [SMILES: CC(=O)Nc1ncc(-c2cnc(C3CC4(CC4)c4cc(-c5c(-n6cnnn6)ccc(Cl)c5F)cc(=O)n43)[nH]2)s1]

LC/MS: mass calculated for C₂₅H₁₉ClFN₉O₂S: 563.11, measured (ES, m/z): 564.05 [M+H]⁺. ¹H NMR (300 MHz, DMSO-d₆): δ 9.37 (s, 1H), 7.85-7.95 (m, 1H), 7.73 (s, 1H), 7.54-7.64 (m, 2H), 6.24 (d, J=1.5 Hz, 1H), 5.95 (dd, J=9.3, 6.1 Hz, 1H), 5.74 (s, 1H), 2.79-2.93 (m, 1H), 2.45-2.60 (m, 1H), 2.25 (s, 3H), 1.20-1.35 (m, 4H). ¹⁹F NMR (282 MHz, DMSO-d₆): δ −74.16, −112.82, −218.49.

### Example 23: (1S,3S)-3-(5-(2-Aminothiazol-5-yl)-1H-imidazol-2-yl)-7-(3-chloro-2-fluoro-6-(1H-tetrazol-1-yl)phenyl)-1-methyl-2,3-dihydroindolizin-5(1H)-one

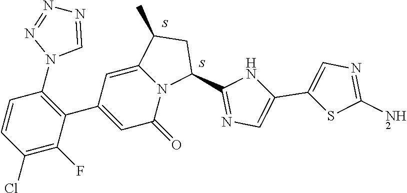

### Step 1: (1S,3S)-7-(3-Chloro-2-fluoro-6-(1H-tetrazol-1-yl)phenyl)-1-methyl-5-oxo-1,2,3,5-tetrahydroindolizine-3-carboxylic acid

To a solution of (1R,3R)-7-(6-amino-3-chloro-2-fluorophenyl)-1-methyl-3-(5-(1-methyl-1H-1,2,3-triazol-5-yl)-1H-imidazol-2-yl)-2,3-dihydroindolizin-5(1H)-one (0.8 g, 2.37 mmol, 1.0 equiv.) and azidotrimethylsilane (4.1 g, 35.64 mmol, 15 equiv.) in CH₃COOH (10 mL) was added trimethoxymethane (3.7 g, 3.18 mmol, 15 equiv.). The reaction mixture was stirred at room temperature overnight, then concentrated under vacuum. The residue was purified by reverse-phase chromatography (C18 column, CH₃CN/H₂O (0.05% TFA)=10%→70%) to yield (1R,3R)-7-(3-chloro-2-fluoro-6-(1H-tetrazol-1-yl)phenyl)-1-methyl-5-oxo-1,2,3,5-tetrahydroindolizine-3-carboxylic as a light yellow acid. LC/MS: mass calculated for C₁₇H₁₃ClFN₅O₃: 389.07, measured (ES, m/z): 390.05 [M+H]⁺.

### Step 2: 2-(2-Aminothiazol-5-yl)-2-oxoethyl (1S,3S)-7-(3-chloro-2-fluoro-6-(1H-tetrazol-1-yl)phenyl)-1-methyl-5-oxo-1,2,3,5-tetrahydroindolizine-3-carboxylate

To a solution of (1R,3R)-7-(3-chloro-2-fluoro-6-(1H-tetrazol-1-yl)phenyl)-1-methyl-5-oxo-1,2,3,5-tetrahydroindolizine-3-carboxylic acid (0.1 g, 0.26 mmol, 1.0 equiv.) in DMF (3 mL) was added K₂CO₃ (0.11 g, 0.77 mmol, 3.0 equiv.) under N₂. The reaction mixture was stirred for 0.5 h, then 1-(2-aminothiazol-5-yl)-2-bromoethan-1-one (0.11 g, 0.51 mmol, 2.0 equiv.) was added. The reaction mixture was stirred 1 h at room temperature and then concentrated under vacuum. The residue was purified by reverse-phase chromatography (C18 column, 330 g, CH₃CN/H₂O (0.05% TFA)=10%→70%) to yield 2-(2-aminothiazol-5-yl)-2-oxoethyl (1R,3R)-7-(3-chloro-2-fluoro-6-(1H-tetrazol-1-yl)phenyl)-1-methyl-5-oxo-1,2,3,5-tetrahydroindolizine-3-carboxylate as a yellow oil. LC/MS: mass calculated for C₂₂H₁₇ClFN₇O₄S: 529.07, measured (ES, m/z): 530.00 [M+H]⁺.

### Step 3: (1S,3S)-3-(5-(2-Aminothiazol-5-yl)-1H-imidazol-2-yl)-7-(3-chloro-2-fluoro-6-(1H-tetrazol-1-yl)phenyl)-1-methyl-2,3-dihydroindolizin-5(1H)-one

To a mixture of 2-(2-aminothiazol-5-yl)-2-oxoethyl (1R,3S)-7-(3-chloro-2-fluoro-6-(1H-tetrazol-1-yl)phenyl)-1-methyl-5-oxo-1,2,3,5-tetrahydroindolizine-3-carboxylate (70 mg, 0.15 mmol, 1.0 equiv.) and ammonium acetate (0.24 g, 3.04 mmol, 20 equiv.) in toluene (6 ml) was added AcOH (2 mL). The reaction mixture was heated with stirring at 90° C. for 2 h, then cooled to room temperature and quenched with water, extracted with ethyl acetate, washed with brine, dried over Na₂SO₄ and concentrated under vacuum. The residue was purified by reverse phase chromatography on C18 (80 g, MeCN/H₂O (0.05% CF₃COOH): 0→45%) to yield (1R,3R)-3-(5-(2-aminothiazol-5-yl)-1H-imidazol-2-yl)-7-(3-chloro-2-fluoro-6-(1H-tetrazol-1-yl)phenyl)-1-methyl-2,3-dihydroindolizin-5(1H)-one as white solid.

LC/MS: mass calculated for C₂₂H₁₇ClFN₉OS: 509.09, measured (ES, m/z): 510.20 [M+H]⁺. ¹H NMR (400 MHz, DMSO-d₆) δ 9.66 (s, 1H), 7.98-8.07 (m, 1H), 7.70-7.76 (m, 1H), 7.51 (s, 1H), 7.37 (d, J=2.4 Hz, 1H), 6.11 (s, 2H), 5.53 (t, J=7.7 Hz, 1H), 3.28-3.41 (m, 1H), 2.70-2.82 (m, 1H), 1.86-1.98 (m, 1H), 1.31 (d, J=7.0 Hz, 3H). ¹⁹F NMR (376 MHz, DMSO-d₆) δ −73.91, −112.92.

### Example 24: (R)-3′-(5-(6-Amino-2-fluoropyridin-3-yl)-1H-imidazol-2-yl)-7′-(3-chloro-2-fluoro-6-(1H-tetrazol-1-yl)phenyl)-2′,3′-dihydro-5′H-spiro[cyclopropane-1,1′-indolizin]-5′-one

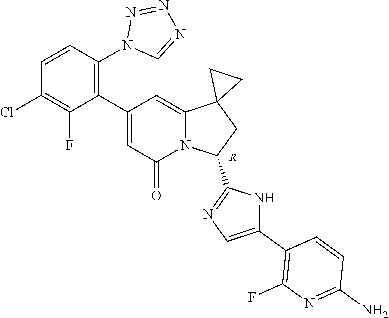

LC/MS: mass calculated for C₂₅H₁₈ClF₂N₉O: 533.13, measured (ES, m/z): 534.15 [M+H]⁺. ¹H NMR (300 MHz, DMSO-d₆): δ 11.97 (s, 1H), 9.64 (d, J=4.8 Hz, 1H), 7.91-8.07 (m, 2H), 7.66-7.76 (m, 1H), 7.06-7.13 (m, 1H), 6.35-6.44 (m, 1H), 6.25 (s, 2H), 6.07 (d, J=8.6 Hz, 1H), 5.65-5.75 (m, 1H), 5.59 (s, 1H), 2.68-2.81 (m, 1H), 2.29 (d, J=12.6 Hz, 1H), 1.09-1.27 (m, 3H), 0.99-1.09 (m, 1H). ¹⁹F NMR (282 MHz, DMSO-d₆): δ−70.32, −70.89, −112.98.

### Example 25: (S)-3′-(5-(6-Amino-2-fluoropyridin-3-yl)-1H-imidazol-2-yl)-7′-(3-chloro-2-fluoro-6-(1H-tetrazol-1-yl)phenyl)-2′,3′-dihydro-5′H-spiro[cyclopropane-1,1′-indolizin]-5′-one

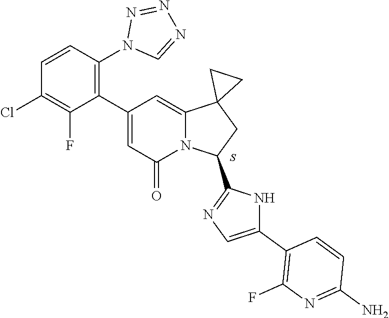

To a solution of N-(5-(2-(7′-(3-chloro-2-fluoro-6-(1H-tetrazol-1-yl)phenyl)-5′-oxo-2′,3′-dihydro-5′H-spiro[cyclopropane-1,1′-indolizin]-3′-yl)-1H-imidazol-5-yl)-6-fluoropyridin-2-yl)acetamide (0.12 g, 0.208 mmol, 1 equiv) in THE (2 mL) was added HCl (1 mL) and the resulting mixture was stirred for 1 h at 50° C. The solvent was evaporated in vacuum and the residue was purified by reverse phase chromatography on C18 (0-50% ACN/H₂O) to yield 3′-(5-(6-amino-2-fluoropyridin-3-yl)-1H-imidazol-2-yl)-7′-(3-chloro-2-fluoro-6-(1H-tetrazol-1-yl)phenyl)-2′,3′-dihydro-5′H-spiro[cyclopropane-1,1′-indolizin]-5′-one. The racemic product was further separated by chiral-HPLC to yield (S)-3′-(5-(6-amino-2-fluoropyridin-3-yl)-1H-imidazol-2-yl)-7′-(3-chloro-2-fluoro-6-(1H-tetrazol-1-yl)phenyl)-2′,3′-dihydro-5′H-spiro[cyclopropane-1,1′-indolizin]-5′-one as a light yellow solid.

LC/MS: mass calculated for C₂₅H₁₈ClF₂N₉O: 533.13, measured (ES, m/z): 534.15 [M+H]⁺. ¹H NMR (300 MHz, DMSO-d₆): δ 9.64 (s, 1H), 7.95-8.06 (m, 2H), 7.70-7.75 (m, 1H), 7.18 (s, 1H), 6.23-6.49 (m, 1H), 6.09 (s, 1H), 5.74 (d, J=9.1 Hz, 1H), 5.62 (s, 1H), 2.65-2.82 (m, 1H), 2.31 (d, J=14.6 Hz, 1H), 1.08-1.21 (m, 2H), 0.91-1.04 (m, 2H). ¹⁹F NMR (282 MHz, DMSO-d₆): δ −70.75, −73.51, −112.97.

### Example 26: (1S,3S)-3-(5-(2-Amino-4-methylthiazol-5-yl)-1H-imidazol-2-yl)-7-(3-chloro-2-fluoro-6-(1H-tetrazol-1-yl)phenyl)-1-methyl-2,3-dihydroindolizin-5(1H)-one

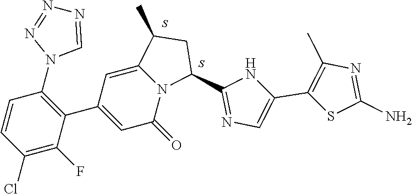

### Step 1: 2-(2-amino-4-methylthiazol-5-yl)-2-oxoethyl (1S,3S)-7-(3-chloro-2-fluoro-6-(1H-tetrazol-1-yl)phenyl)-1-methyl-5-oxo-1,2,3,5-tetrahydroindolizine-3-carboxylate

To a solution of (1S,3S)-7-(3-chloro-2-fluoro-6-(1H-tetrazol-1-yl)phenyl)-1-methyl-5-oxo-1,2,3,5-tetrahydroindolizine-3-carboxylic acid (100 mg, 0.26 mmol, 1 equiv.) in CH₃CN (5 mL) was added potassium carbonate (106 mg, 0.77 mmol, 3 equiv). After the reaction mixture was stirred at room temperature for 30 min, 1-(2-amino-4-methylthiazol-5-yl)-2-bromoethan-1-one (90 mg, 0.38 mmol, 1.5 equiv) was added. The reaction mixture was stirred 5 h at room temperature, then was quenched with water (10 mL) and extracted with ethyl acetate (20 mL×3). The combined organic layer was washed with brine, dried over Na₂SO₄. The solids were filtered out. The resulting organic phase was concentrated under vacuum. The residue was applied onto a silica gel column (PE/EA=3:1) to yield 2-(2-amino-4-methylthiazol-5-yl)-2-oxoethyl (1S,3S)-7-(3-chloro-2-fluoro-6-(1H-tetrazol-1-yl)phenyl)-1-methyl-5-oxo-1,2,3,5-tetrahydroindolizine-3-carboxylate as a yellow solid. LC/MS: mass calculated for C₂₃H₁₉ClFN₇O₄S: 543.09, measured: 544.05 [M+H]⁺.

### Step 2: (1S,3S)-3-(5-(2-Amino-4-methylthiazol-5-yl)-1H-imidazol-2-yl)-7-(3-chloro-2-fluoro-6-(1H-tetrazol-1-yl)phenyl)-1-methyl-2,3-dihydroindolizin-5(1H)-one

To a solution of 2-(2-amino-4-methylthiazol-5-yl)-2-oxoethyl (1S,3S)-7-(3-chloro-2-fluoro-6-(1H-tetrazol-1-yl)phenyl)-1-methyl-5-oxo-1,2,3,5-tetrahydroindolizine-3-carboxylate (50 mg, 0.092 mmol, 1 eq.) in toluene (20 mL) were added ammonium acetate (106 mg, 1.379 mmol, 15 eq.) and glacial acetic acid (55 mg, 0.92 mmol, 10 eq.). The reaction mixture was stirred at 100° C. for 1 h, then concentrated, and the residue was purified by silica gel chromatography (0→10% MeOH/DCM) to yield (1S,3S)-3-(5-(2-amino-4-methylthiazol-5-yl)-1H-imidazol-2-yl)-7-(3-chloro-2-fluoro-6-(1H-tetrazol-1-yl)phenyl)-1-methyl-2,3-dihydroindolizin-5(1H)-one as an off-white solid.

LC/MS: mass calculated for C₂₃H₁₉ClFN₉OS: 523.11, measured (ES, m/z): 524.05 [M+H]⁺. ¹H NMR (400 MHz, DMSO-d₆) δ 12.80 (brs., 1H), 9.65 (s, 1H), 8.86 (s, 1H), 7.98-8.06 (m, 1H), 7.70-7.80 (m, 1H), 7.42 (s, 1H), 5.91-6.19 (m, 2H), 5.46-5.58 (m, 1H), 3.25-3.45 (m, 1H), 2.72-2.85 (m, 1H), 2.28 (s, 3H), 1.88-1.95 (m, 1H), 1.32 (d, J=7.0 Hz, 3H). ¹⁹F NMR (376 MHz, DMSO-d₆) δ−74.30, −113.03.

### Example 27: (1S,3S)-3-(5-(2-Amino-4-(trifluoromethyl)thiazol-5-yl)-1H-imidazol-2-yl)-7-(3-chloro-2-fluoro-6-(1H-tetrazol-1-yl)phenyl)-1-methyl-2,3-dihydroindolizin-5(1H)-one

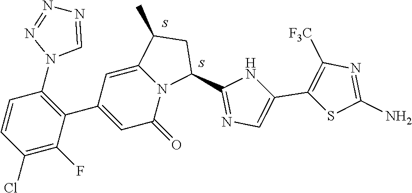

LC/MS: mass calculated for C₂₃H₁₆ClF₄N₉OS: 577.08, measured (ES, m/z): 578.25 [M+H]⁺. ¹H NMR (400 MHz, DMSO-d₆) δ 9.66 (s, 1H), 7.98-8.06 (m, 1H), 7.70-7.80 (m, 1H), 7.30-7.68 (m, 3H), 6.05-6.15 (m, 2H), 5.55 (s, 1H), 3.33-3.41 (m, 1H), 2.70-2.86 (m, 1H), 1.90-2.00 (m, 1H), 1.32 (d, J=7.0 Hz, 3H). ¹⁹F NMR (376 MHz, DMSO-d₆) δ −60.33, −74.69, −112.86.

### Example 28: (1S,3S)-7-(3-Chloro-2-fluoro-6-(1H-tetrazol-1-yl)phenyl)-1-methyl-3-(5-(1-methyl-1H-1,2,3-triazol-5-yl)-1H-Imidazol-2-yl)-2,3-dihydroindolizin-5(1H)-one

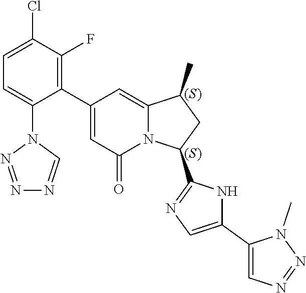

LC/MS: mass calculated for C₂₂H₁₈ClFN₁₀O: 492.13, measured (ES, m/z): 493.15 [M+H]⁺. ¹H NMR (400 MHz, Methanol-d₄) δ 9.38 (s, 1H), 7.85-7.95 (m, 2H), 7.66 (s, 1H), 7.58-7.62 (m, 1H), 6.26 (s, 1H), 6.19 (s, 1H), 5.70 (t, J=7.8 Hz, 1H), 4.23 (s, 3H), 3.46 (d, J=7.6 Hz, 1H), 2.85-2.95 (m, 1H), 2.10-2.22 (m, 1H), 1.42 (d, J=7.0 Hz, 3H). ¹⁹F NMR (376 MHz, Methanol-d₄) δ −77.43, −113.94.

### Example 29: (1S,3S)-3-[5-(6-amino-2-fluoro-3-pyridyl)-1H-imidazol-2-yl]-7-[5-chloro-2-(tetrazol-1-yl)phenyl]-1-methyl-2,3-dihydro-1H-indolizin-5-one

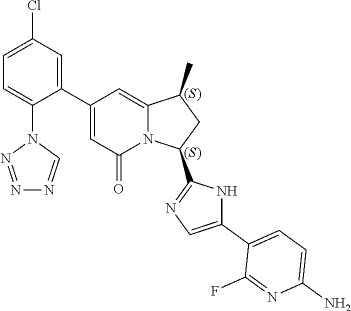

### Step 1: 2-(6-Amino-2-fluoropyridin-3-yl)-2-oxoethyl (1S,3S)-7-(5-chloro-2-(1H-tetrazol-1-yl)phenyl)-1-methyl-5-oxo-1,2,3,5-tetrahydroindolizine-3-carboxylate

To a solution of (1S, 3S)-7-(5-chloro-2-(1H-tetrazol-1-yl)phenyl)-1-methyl-5-oxo-1,2,3,5-tetrahydroindolizine-3-carboxylic acid (120 mg, 0.32 mmol, 1 equiv.) in DMF (6 mL) was added K₂CO₃ (134 mg, 0.96 mmol, 3 equiv.) under N₂. The reaction mixture was stirred for 0.5 h, and then a solution of 1-(6-amino-2-fluoropyridin-3-yl)-2-bromoethan-1-one (113 mg, 0.48 mmol, 1.5 equiv) was added. The reaction mixture was stirred 1 h at room temperature, then concentrated under vacuum. The residue was purified by silica gel chromatography with EA/PE (0→100%) to 2-(6-amino-2-fluoropyridin-3-yl)-2-oxoethyl (1S,3S)-7-(5-chloro-2-(1H-tetrazol-1-yl)phenyl)-1-methyl-5-oxo-1,2,3,5-tetrahydroindolizine-3-carboxylate as white solid.

### Step 2: (1S,3S)-3-(5-(6-Amino-2-fluoropyridin-3-yl)-1H-imidazol-2-yl)-7-(5-chloro-2-(1H-tetrazol-1-yl)phenyl)-1-methyl-2,3-dihydroindolizin-5(1H)-one

To a solution of 2-(6-amino-2-fluoropyridin-3-yl)-2-oxoethyl (1S,3S)-7-(5-chloro-2-(1H-tetrazol-1-yl)phenyl)-1-methyl-5-oxo-1,2,3,5-tetrahydroindolizine-3-carboxylate (120 g, 0.28 mmol, 1 equiv.) in toluene (5 mL) and AcOH (0.5 mL) was added ammonium acetate (441 mg, 5.72 mol, 20 equiv). The reaction mixture was stirred 2 h at 90° C., then cooled to room temperature and quenched with water, extracted with EA, washed with brine, dried over Na₂SO₄ and concentrated under vacuum. The residue was purified by silica gel chromatography with EA/PE (0→66%) to yield (1S,3S)-3-(5-(6-amino-2-fluoropyridin-3-yl)-1H-imidazol-2-yl)-7-(5-chloro-2-(1H-tetrazol-1-yl)phenyl)-1-methyl-2,3-dihydroindolizin-5(1H)-one as yellow oil, which was further purified by prep-HPLC to yield (1S,3S)-3-(5-(6-amino-2-fluoropyridin-3-yl)-1H-imidazol-2-yl)-7-(5-chloro-2-(1H-tetrazol-1-yl) phenyl)-1-methyl-2,3-dihydroindolizin-5(1H)-one.

LC/MS: mass calculated for C₂₄H₁₉ClFN₉O: 503.14, measured (ES, m/z): 504.15 [M+H]⁺. ¹H NMR (300 MHz, DMSO-d₆) δ 9.72 (s, 1H), 8.02 (d, J=2.2 Hz, 1H), 7.93 (s, 1H), 7.86 (dd, J=8.4, 2.2 Hz, 1H), 7.73 (d, J=8.4 Hz, 1H), 6.99-7.30 (m, 1H). 6.39 (d, J=8.3 Hz, 1H), 6.20-6.38 (m 1H). 6.00 (d, J=2.0 Hz, 1H), 5.81 (s, 1H), 5.52 (d, J=7.7 Hz, 1H), 3.33 (s, 1H), 2.62-2.95 (m, 1H), 2.01 (s, 1H), 1.29 (d, J=7.0 Hz, 3H).

### Example 30: 4-(2-((1S,3R)-7-(3-Chloro-2-fluoro-6-(1H-tetrazol-1-yl)phenyl)-1-methyl-5-oxo-1,2,3,5-tetrahydroindolizin-3-yl)-1H-imidazol-5-yl)picolinonitrile

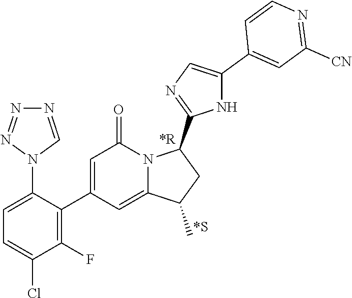 [SMILES: C[C@H]1C[C@H](c2ncc(-c3ccnc(C#N)c3)[nH]2)n2c1cc(-c1c(-n3cnnn3)ccc(Cl)c1F)cc2=O]

LC/MS: mass calculated for C₂₅H₁₇ClFN₉O: 513.12, measured (ES, m/z): 514.10 [M+H]⁺. ¹H NMR (400 MHz, DMSO-d₆) δ 12.17-13.17 (m, 1H), 9.67 (s, 1H), 8.64 (d, J=5.2 Hz, 1H), 8.28 (d, J=1.7 Hz, 1H), 7.91-8.08 (m, 3H), 7.75 (dd, J=8.7, 1.5 Hz, 1H), 6.16 (s, 1H), 6.03 (d, J=1.7 Hz, 1H), 5.64 (d, J=8.7 Hz, 1H), 3.47-3.80 (m, 1H), 2.43-2.50 (m, 1H), 1.92-2.04 (m, 1H), 1.20 (d, J=6.8 Hz, 3H). ¹⁹F NMR (376 MHz, DMSO-d₆) δ −74.51, −113.14.

### Example 31: 4-(2-((1S*,3R*)-7-(3-Chloro-2-fluoro-6-(1H-tetrazol-1-yl)phenyl)-1-methyl-5-oxo-1,2,3,5-tetrahydroindolizin-3-yl)-1H-imidazol-5-yl)-3-methylpicolinonitrile

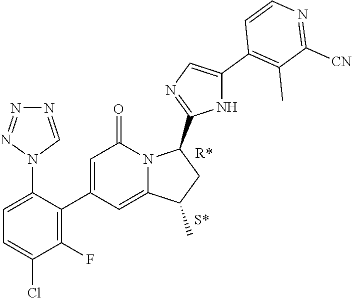 [SMILES: Cc1c(-c2cnc([C@H]3C[C@H](C)c4cc(-c5c(-n6cnnn6)ccc(Cl)c5F)cc(=O)n43)[nH]2)ccnc1C#N]

LC/MS: mass calculated for C₂₆H₁₉ClFN₉O: 527.14, measured (ES, m/z): 528.15 [M+H]⁺. ¹H NMR (400 MHz, DMSO-d₆) δ 12.78 (brs., 1H), 9.67 (s, 1H), 8.53 (d, J=5.1 Hz, 1H), 7.96-8.06 (m, 2H), 7.69-7.78 (m, 2H), 6.17 (s, 1H), 6.05 (s, 1H), 5.68 (d, J=8.6 Hz, 1H), 3.60-3.75 (m, 1H), 2.64 (s, 3H), 2.42-2.50 (m, 1H), 2.12-2.25 (m, 1H), 1.21 (d, J=6.8 Hz, 3H). ¹⁹F NMR (376 MHz, DMSO-d₆) δ −74.68, −113.18.

### Example 32: 4-(2-((1S,3S)-7-(3-Chloro-2-fluoro-6-(1H-tetrazol-1-yl)phenyl)-1-methyl-5-oxo-1,2,3,5-tetrahydroindolizin-3-yl)-1_H-imidazol-5-yl)picolinonitrile

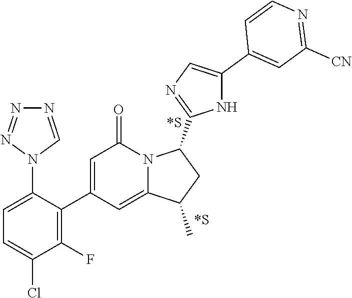 [SMILES: C[C@H]1C[C@@H](c2ncc(-c3ccnc(C#N)c3)[nH]2)n2c1cc(-c1c(-n3cnnn3)ccc(Cl)c1F)cc2=O]

LC/MS: mass calculated for C₂₅H₁₇ClFN₉O: 513.12, measured (ES, m/z): 514.10 [M+H]⁺. ¹H NMR (400 MHz, DMSO-d₆) δ 9.65 (s, 1H), 8.65 (d, J=5.4 Hz, 1H), 8.28 (d, J=1.7 Hz, 1H), 7.92-8.10 (m, 3H), 7.74 (dd, J=8.7, 1.5 Hz, 1H), 6.10 (s, 1H), 6.05 (s, 1H), 5.54 (dd, J=8.6, 5.7 Hz, 1H), 3.36 (q, J=7.3 Hz, 1H), 2.71-2.84 (m, 1H), 1.93-2.03 (m, 1H), 1.31 (d, J=7.1 Hz, 3H), 1.24 (s, 1H). ¹⁹F NMR (376 MHz, DMSO-d₆) δ −74.21, −113.11.

### Example 33: 4-(2-((1S*,3S*)-7-(3-Chloro-2-fluoro-6-(1H-tetrazol-1-yl)phenyl)-1-methyl-5-oxo-1,2,3,5-tetrahydroindolizin-3-yl)-1H-imidazol-5-yl)-3-methylpicolinonitrile

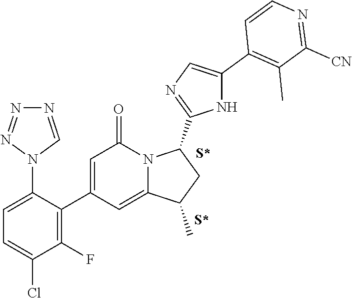 [SMILES: Cc1c(-c2cnc([C@@H]3C[C@H](C)c4cc(-c5c(-n6cnnn6)ccc(Cl)c5F)cc(=O)n43)[nH]2)ccnc1C#N]

LC/MS: mass calculated for C₂₆H₁₉ClFN₉O: 527.14, measured (ES, m/z): 528.15 [M+H]⁺. ¹H NMR (400 MHz, DMSO-d₆) δ 12.92 (brs., 1H), 9.65 (s, 1H), 8.55 (d, J=5.1 Hz, 1H), 7.97-8.06 (m, 2H), 7.70-7.78 (m, 2H), 6.12 (s, 1H), 6.06 (s, 1H), 5.59 (dd, J=8.7, 5.5 Hz, 1H), 3.30-3.40 (m, 1H), 2.75-2.90 (m, 1H), 2.66 (s, 3H), 1.90-2.10 (m, 1H), 1.31 (d, J=7.1 Hz, 3H). ¹⁹F NMR (376 MHz, DMSO-d₆) δ −74.53, −113.14.

### Example 34: (1S,3S)-3-(2-(6-Amino-2-fluoropyridin-3-yl)-4-fluoro-1H-imidazol-5-yl)-7-(3-chloro-2-fluoro-6-(1H-tetrazol-1-yl)phenyl)-1-methyl-2,3-dihydroindolizin-5(1H)-one

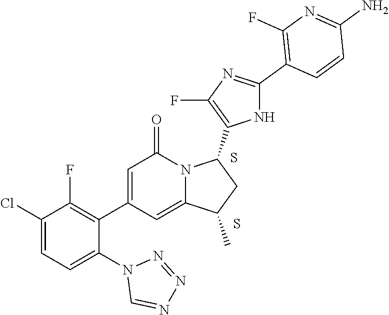

### Step 1: Tert-butyl (5-(5-(7-(3-chloro-2-fluoro-6-(1H-tetrazol-1-yl)phenyl)-1-methyl-5-oxo-1,2,3,5-tetrahydroindolizin-3-yl)-1H-imidazol-2-yl)-6-fluoropyridin-2-yl)carbamate

To a solution of tert-butyl (5-(5-(7-(3-chloro-2-fluoro-6-(1H-tetrazol-1-yl)phenyl)-1-methylene-5-oxo-1,2,3,5-tetrahydroindolizin-3-yl)-1H-imidazol-2-yl)-6-fluoropyridin-2-yl)carbamate (0.1 g, 0.16 mmol, 1.0 equiv.) in MeOH (10 mL) and chlorobenzene (2 mL) was added 5% Pd/C (0.05 g), then hydrogen was introduced in. The reaction mixture was stirred for 30 min at room temperature. Pd/C was filtered out and the resulting filtrate was evaporated under vacuum. The residue was purified by silica gel chromatography with MeOH/DCM (0→10%) to yield tert-butyl (5-(5-(7-(3-chloro-2-fluoro-6-(1H-tetrazol-1-yl)phenyl)-1-methyl-5-oxo-1,2,3,5-tetrahydroindolizin-3-yl)-1H-imidazol-2-yl)-6-fluoropyridin-2-yl)carbamate as a light yellow solid.

### Step 2: Tert-butyl (5-(5-(7-(3-chloro-2-fluoro-6-(1H-tetrazol-1-yl)phenyl)-1-methyl-5-oxo-1,2,3,5-tetrahydroindolizin-3-yl)-4-fluoro-1H-imidazol-2-yl)-6-fluoropyridin-2-yl)carbamate

To a solution of tert-butyl (5-(5-(7-(3-chloro-2-fluoro-6-(1H-tetrazol-1-yl)phenyl)-1-methyl-5-oxo-1,2,3,5-tetrahydroindolizin-3-yl)-1H-imidazol-2-yl)-6-fluoropyridin-2-yl)carbamate (65 mg, 0.10 mmol, 1 equiv.) in DCM (3 mL) and acetone (3 mL) was added NaHCO₃ (44 mg, 0.52 mmol, 5 equiv.) followed by addition of N-fluoro-N-(phenylsulfonyl)benzenesulfonamide (99 mg, 0.313 mmol, 3 equiv.). The reaction mixture was stirred at 50° C. for 3 h, then cooled to room temperature and purified by reverse column chromatography with CH₃CN/water (5%→50%) to yield tert-butyl (5-(5-(7-(3-chloro-2-fluoro-6-(1H-tetrazol-1-yl)phenyl)-1-methyl-5-oxo-1,2,3,5-tetrahydroindolizin-3-yl)-4-fluoro-1H-imidazol-2-yl)-6-fluoropyridin-2-yl)carbamate as light yellow oil.

### Step 3: (1S,3S)-3-(2-(6-Amino-2-fluoropyridin-3-yl)-4-fluoro-1H-imidazol-5-yl)-7-(3-chloro-2-fluoro-6-(1H-tetrazol-1-yl)phenyl)-1-methyl-2,3-dihydroindolizin-5(1H)-one

To a solution of tert-butyl (5-(5-(7-(3-chloro-2-fluoro-6-(1H-tetrazol-1-yl)phenyl)-1-methyl-5-oxo-1,2,3,5-tetrahydroindolizin-3-yl)-4-fluoro-1H-imidazol-2-yl)-6-fluoropyridin-2-yl)carbamate (15 mg, 0.023 mmol, 1.0 equiv.) in DCM (5 mL) was added TFA (1 mL). The reaction was stirred for 1 h at room temperature, then concentrated under vacuum. The residue was purified by prep-Chiral-HPLC with (Hex:DCM=3:1)(0.1% DEA):EtOH=50:50 to yield (1S,3S)-3-(2-(6-amino-2-fluoropyridin-3-yl)-4-fluoro-1H-imidazol-5-yl)-7-(3-chloro-2-fluoro-6-(1H-tetrazol-1-yl)phenyl)-1-methyl-2,3-dihydroindolizin-5(1H)-one as a white solid.

LC/MS: mass calculated for C₂₄H₁₇ClF₃N₉O: 539.12, measured (ES, m/z): 540.20 [M+H]⁺. ¹H NMR (400 MHz, Methanol-d₄): δ 9.34 (s, 1H), 7.84-7.98 (m, 2H), 7.60 (dd, J=8.6, 1.6 Hz, 1H), 6.48 (dd, J=8.4, 2.0 Hz, 1H), 6.31 (s, 1H), 6.10 (s, 1H), 5.68-5.77 (m, 1H), 3.38-3.47 (m, 1H), 2.79-2.90 (m, 1H), 2.17-2.27 (m, 1H), 1.33 (d, J=7.2 Hz, 3H). ¹⁹F NMR (376 MHz, Methanol-d₄): δ −73.52, −76.96, −114.03, 138.29.

### Example 35: (1*S,3*S)-3-(2-(6-Amino-2-fluoropyridin-3-yl)-4-fluoro-1H-imidazol-5-yl)-7-(3-chloro-2-fluoro-6-(1H-tetrazol-1-yl)phenyl)-1-methyl-2,3-dihydroindolizin-5(1H)-one

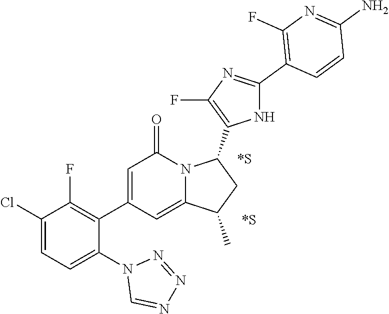

LC/MS: mass calculated for C₂₄H₁₇ClF₃N₉O: 539.12, measured (ES, m/z): 540.20 [M+H]^{+ 1}H NMR (400 MHz, Methanol-d₄): δ 9.34 (s, 1H), 7.85-7.99 (m, 2H), 7.60 (dd, J=8.7, 1.7 Hz, 1H), 6.48 (dd, J=8.4, 2.0 Hz, 1H), 6.31 (s, 1H), 6.10 (s, 1H), 5.72 (dd, J=8.7, 5.7 Hz, 1H), 3.37-3.49 (m, 1H), 2.78-2.92 (m, 1H), 2.17-2.29 (m, 1H), 1.33 (d, J=7.1 Hz, 3H). ¹⁹F NMR (376 MHz, Methanol-d₄): δ −73.52, −76.99, −114.03, 138.29

### Example 36: (1S,3S)-3-(5-(6-Amino-2-fluoropyridin-3-yl)-1H-imidazol-2-yl)-7-(5-chloro-2-(1H-tetrazol-1-yl)phenyl)-1-methyl-2,3-dihydroindolizin-5(1H)-one

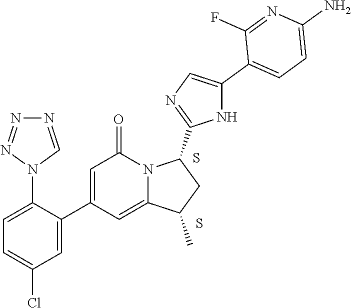

LC/MS: mass calculated for C₂₄H₁₉ClFN₉O: 503.14, measured (ES, m/z): 504.20 [M+H]⁺. ¹H NMR (300 MHz, Methanol-d₄) δ 9.38 (s, 1H), 7.92-8.05 (m, 1H), 7.66-7.81 (m, 3H), 7.20 (d, J=3.4 Hz, 1H), 6.48 (dd, J=8.3, 1.9 Hz, 1H), 6.21-6.23 (m, 1H), 5.95-5.97 (m, 1H), 5.62 (t, J=7.7 Hz, 1H), 3.37-3.42 (m, 1H), 2.79-2.92 (m, 1H), 2.08-2.24 (m, 1H), 1.36 (d, J=7.0 Hz, 3H).

### Example 37: (1*S,3*R)-3-(5-(6-Amino-2-chloropyridin-3-yl)-1H-imidazol-2-yl)-7-(3-chloro-2-fluoro-6-(1H-tetrazol-1-yl)phenyl)-1-methyl-2,3-dihydroindolizin-5(1H)-one

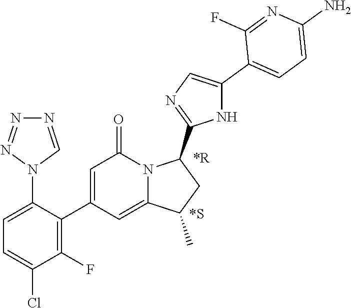

LC/MS: mass calculated for C₂₄H₁₈Cl₂FN₉O: 537.10, measured (ES, m/z): 538.05 [M+H]⁺. ¹H NMR (300 MHz, DMSO-d₆): δ 12.08-12.29 (m, 1H), 9.67 (s, 1H), 8.05-7.98 (m, 1H), 7.94 (d, J=8.4 Hz, 1H), 7.74 (dd, J=8.7, 1.5 Hz, 1H), 7.40 (d, J=1.7 Hz, 1H), 6.50 (d, J=8.5 Hz, 1H), 6.32 (s, 2H), 6.17 (s, 1H), 6.02 (s, 1H), 5.62 (d, J=8.6 Hz, 1H), 3.57-3.78 (m, 1H), 2.41-2.52 (m, 1H), 2.04-2.21 (m, 1H), 1.19 (d, J=6.8 Hz, 3H). ¹⁹F NMR (282 MHz, DMSO-d₆): δ−113.12.

### Example 38: (1*R,3*R)-3-(5-(2-(Aminomethyl)pyridin-4-yl)-1H-imidazol-2-yl)-7-(3-chloro-2-fluoro-6-(1H-tetrazol-1-yl)phenyl)-1-methyl-2,3-dihydroindolizin-5(1H)-one

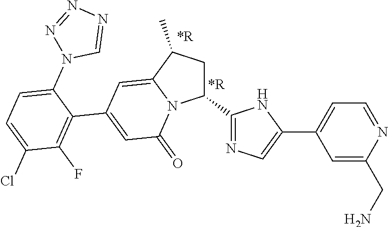 [SMILES: C[C@@H]1C[C@H](c2ncc(-c3ccnc(CN)c3)[nH]2)n2c1cc(-c1c(-n3cnnn3)ccc(Cl)c1F)cc2=O]

To a solution of (*R)-4-(2-(7-(3-chloro-2-fluoro-6-(1H-tetrazol-1-yl)phenyl)-1-methylene-5-oxo-1,2,3,5-tetrahydroindolizin-3-yl)-1H-imidazol-5-yl)picolinonitrile (20.0 mg, 0.039 mmol) in CH₃OH/chlorobenzene (6.0 mL) was added Pd/C (41.5 mg, 0.039 mmol, 10%). The mixture stirred at room temperature for 20 min under H₂ atmosphere. The reaction was concentrated and the residue was purified by reverse phase chromatography on C18 (0-40% CH₃CN/H₂O) to yield (1*R,3*R)-3-(5-(2-(aminomethyl)pyridin-4-yl)-1H-imidazol-2-yl)-7-(3-chloro-2-fluoro-6-(1H-tetrazol-1-yl)phenyl)-1-methyl-2,3-dihydroindolizin-5(1H)-one as a brown solid.

LC/MS: mass calculated for C₂₅H₂₁ClFN₉O: 517.15, measured (ES, m/z): 518.15 [M+H]⁺. ¹H NMR (300 MHz, Methanol-d₄) δ 9.38 (s, 1H), 8.56 (d, J=5.4 Hz, 1H), 7.87 (dd, J=8.7, 7.6 Hz, 1H), 7.79 (s, 1H), 7.75 (s, 1H), 7.73-7.64 (m, 1H), 7.59 (dd, J=8.7, 1.7 Hz, 1H), 6.20 (m 2H), 5.63 (t, J=7.8 Hz, 1H), 4.32 (s, 2H), 3.40-3.50 (m, 1H), 2.83-2.94 (m, 1H), 2.08-2.18 (m, 1H), 1.40 (d, J=7.0 Hz, 3H). ¹⁹F NMR (282 MHz, Methanol-d₄) δ −77.11, −113.92.

### Example 39: (1*S,3*S)-3-(5-(2-(Aminomethyl)pyridin-4-yl)-1H-imidazol-2-yl)-7-(3-chloro-2-fluoro-6-(1H-tetrazol-1-yl)phenyl)-1-methyl-2,3-dihydroindolizin-5(1H)-one

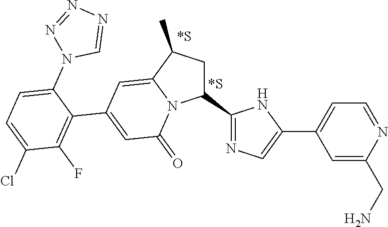

LC/MS: mass calculated for C₂₅H₂₁ClFN₉O: 517.15, measured (ES, m/z): 518.15 [M+H]⁺. ¹H NMR (300 MHz, Methanol-d₄) δ 9.38 (s, 1H), 8.52-8.60 (m, 1H), 7.88 (dd, J=8.7, 7.6 Hz, 1H), 7.78-7.80 (m, 1H), 7.76 (s, 1H), 7.64-7.74 (m, 1H), 7.59 (dd, J=8.7, 1.7 Hz, 1H), 6.16-6.23 (m, 2H), 5.63 (t, J=7.8 Hz, 1H), 4.32 (s, 2H), 3.45 (q, J=7.6 Hz, 1H), 2.81-3.02 (m, 1H), 2.81-3.02 (m, 1H), 1.40 (d, J=7.0 Hz, 3H). ¹⁹F NMR (282 MHz, Methanol-d₄) δ −77.17, −113.93.

### Example 40: (1*S,3*S)-3-(5-(6-Aminopyridin-3-yl)-4-fluoro-1H-imidazol-2-yl)-7-(3-chloro-2-fluoro-6-(1H-tetrazol-1-yl)phenyl)-1-methyl-2,3-dihydroindolizin-5(1H)-one

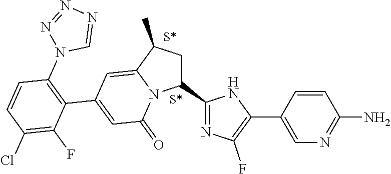

### Step 1: 2-(6-Acetamidopyridin-3-yl)-2-oxoethyl 7-(3-chloro-2-fluoro-6-(1H-tetrazol-1-yl)phenyl)-1-methylene-5-oxo-1,2,3,5-tetrahydroindolizine-3-carboxylate

To a solution of 7-(3-chloro-2-fluoro-6-(1H-tetrazol-1-yl)phenyl)-1-methylene-5-oxo-1,2,3,5-tetrahydroindolizine-3-carboxylic acid (450 mg, 1.16 mmol, 1.0 equiv.) in DMF (8 mL) was added potassium carbonate (481 mg, 3.48 mmol, 3 equiv.). After the mixture was stirred for 30 min, 2-bromo-1-(2-methyl-2H-indazol-5-yl)ethan-1-one (358 mg, 1.39 mmol, 1.2 equiv.) in DMF (2 mL) was added. The reaction mixture was stirred for 2 h at room temperature, then partitioned between water and EtOAc. The organic layer was separated, washed with brine, dried and concentrated. The residue was purified by reverse column chromatography with CH₃CN/0.05% TFA water (5%→80%) to yield 2-(6-acetamidopyridin-3-yl)-2-oxoethyl 7-(3-chloro-2-fluoro-6-(1H-tetrazol-1-yl)phenyl)-1-methylene-5-oxo-1,2,3,5-tetrahydroindolizine-3-carboxylate as a light yellow solid.

### Step 2: N-(5-(2-(7-(3-Chloro-2-fluoro-6-(1H-tetrazol-1-yl)phenyl)-1-methylene-5-oxo-1,2,3,5-tetrahydroindolizin-3-yl)-1H-imidazol-5-yl)pyridin-2-yl)acetamide

To a solution of 2-(6-acetamidopyridin-3-yl)-2-oxoethyl 7-(3-chloro-2-fluoro-6-(1H-tetrazol-1-yl)phenyl)-1-methylene-5-oxo-1,2,3,5-tetrahydroindolizine-3-carboxylate (0.45 g, 0.79 mmol, 1.0 equiv.) in toluene (30 mL) was added ammonium acetate (923 mg, 11.97 mmol, 15 equiv.) followed by addition of acetic acid (1 mL). The reaction mixture was stirred for 1 h at 100° C. under N₂, then cooled to room temperature and concentrated under vacuum. The residue was purified by silica gel chromatography with DCM/MeOH (0→10%) to yield N-(5-(2-(7-(3-chloro-2-fluoro-6-(1H-tetrazol-1-yl)phenyl)-1-methylene-5-oxo-1,2,3,5-tetrahydroindolizin-3-yl)-1H-imidazol-5-yl)pyridin-2-yl)acetamide as a light yellow solid.

### Step 3: 3-(5-(6-Aminopyridin-3-yl)-1H-imidazol-2-yl)-7-(3-chloro-2-fluoro-6-(1H-tetrazol-1-yl)phenyl)-1-methylene-2,3-dihydroindolizin-5(1H)-one

To a solution of N-(5-(2-(7-(3-chloro-2-fluoro-6-(1H-tetrazol-1-yl)phenyl)-1-methylene-5-oxo-1,2,3,5-tetrahydroindolizin-3-yl)-1H-imidazol-5-yl)pyridin-2-yl)acetamide (0.24 g, 0.44 mmol, 1 equiv.) in THE (10 mL) was added 1N HCl (10 mL). The reaction mixture was stirred for 3 h at 55° C., then cooled to room temperature and concentrated under vacuum. The residue was purified by reverse column chromatography with CH₃CN/water (5%→50%) to yield 3-(5-(6-aminopyridin-3-yl)-1H-imidazol-2-yl)-7-(3-chloro-2-fluoro-6-(1H-tetrazol-1-yl)phenyl)-1-methylene-2,3-dihydroindolizin-5(1H)-one as an off-white solid.

### Step 4: 3-(5-(6-Aminopyridin-3-yl)-4-fluoro-1H-imidazol-2-yl)-7-(3-chloro-2-fluoro-6-(1H-tetrazol-1-yl)phenyl)-1-methylene-2,3-dihydroindolizin-5(1H)-one

To a solution of 3-(5-(6-aminopyridin-3-yl)-1H-imidazol-2-yl)-7-(3-chloro-2-fluoro-6-(1H-tetrazol-1-yl)phenyl)-1-methylene-2,3-dihydroindolizin-5(1H)-one (0.19 g, 0.37 mmol, 1 equiv.) in DCM (10 ml) and acetone (10 ml) was added NaHCO₃ (0.127 g, 1.514 mmol, 4 equiv.) followed by N-fluoro-N-(phenylsulfonyl)benzenesulfonamide (0.239 g, 0.75 mmol, 2 equiv.). The reaction mixture was stirred at 50° C. for 3 h, then purified by reverse column chromatography with CH₃CN/water (5%→60%) to yield 3-(5-(6-aminopyridin-3-yl)-4-fluoro-1H-imidazol-2-yl)-7-(3-chloro-2-fluoro-6-(1H-tetrazol-1-yl)phenyl)-1-methylene-2,3-dihydroindolizin-5(1H)-one as a brown solid.

### Step 5: (1*S,3*S)-3-(5-(6-Aminopyridin-3-yl)-4-fluoro-1H-imidazol-2-yl)-7-(3-chloro-2-fluoro-6-(1H-tetrazol-1-yl)phenyl)-1-methyl-2,3-dihydroindolizin-5(1H)-one

To a solution of 3-(5-(6-aminopyridin-3-yl)-4-fluoro-1H-imidazol-2-yl)-7-(3-chloro-2-fluoro-6-(1H-tetrazol-1-yl)phenyl)-1-methylene-2,3-dihydroindolizin-5(1H)-one (0.06 g, 0.15 mmol, 1.0 equiv.) in MeOH (10 mL) and chlorobenzene (2 mL) was added 5% Pd/C (0.05 g), then hydrogen was introduced in. The reaction mixture was stirred for 20 min at room temperature. Pd/C was filtered out and the resulting solution was evaporated under vacuum. The residue was purified by reverse column chromatography with 0.1% FA CH₃CN/0.1% FA water (5%→40%) to yield a residue. The racemic mixture (residue) was purified by Prep-Chiral-HPLC with Hex(0.1% DEA):EtOH=50:50 to yield (1*S,3*S)-3-(5-(6-aminopyridin-3-yl)-4-fluoro-1H-imidazol-2-yl)-7-(3-chloro-2-fluoro-6-(1H-tetrazol-1-yl)phenyl)-1-methyl-2,3-dihydroindolizin-5(1H)-one as a white solid.

LC/MS: mass calculated for C₂₄H₁₈ClF₂N₉O: 521.13, measured (ES, m/z): 522.10 [M+H]⁺. ¹H NMR (300 MHz, Methanol-d₄): δ 9.37 (s, 1H), 8.14 (s, 1H), 7.88 (t, J=8.1 Hz, 1H), 7.68 (d, J=8.5 Hz, 1H), 7.59 (d, J=8.6 Hz, 1H), 6.67 (d, J=8.8 Hz, 1H), 6.23 (s, 1H), 6.12 (s, 1H), 5.40-5.55 (m, 1H), 3.32-3.47 (m, 1H), 2.79-2.94 (m, 1H), 2.01-2.16 (m, 1H), 1.40 (d, J=7.0 Hz, 3H). ¹⁹F NMR (282 MHz, Methanol-d₄): δ −113.74, −139.22.

### Example 41: (1*R,3*S)-7-(3-Chloro-2-fluoro-6-(1H-tetrazol-1-yl)phenyl)-1-methyl-3-(5-(2-methylpyridin-4-yl)-1H-imidazol-2-yl)-2,3-dihydroindolizin-5(1H)-one

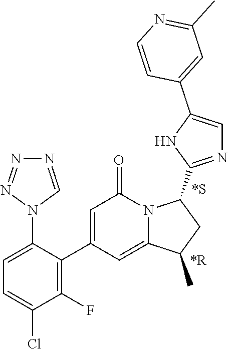 [SMILES: Cc1cc(-c2cnc([C@@H]3C[C@@H](C)c4cc(-c5c(-n6cnnn6)ccc(Cl)c5F)cc(=O)n43)[nH]2)ccn1]

LC/MS: mass calculated for C₂₅H₂₀ClFN₈O: 502.14, measured (ES, m/z): 503.10 [M+H]⁺. ¹H NMR (400 MHz, Methanol-d₄) δ 9.50 (s, 1H), 8.81 (d, J=6.4 Hz, 1H), 8.62 (s, 1H), 8.40 (d, J=1.9 Hz, 1H), 8.31 (dd, J=6.5, 1.9 Hz, 1H), 7.92 (dd, J=8.7, 7.6 Hz, 1H), 7.62 (dd, J=8.7, 1.6 Hz, 1H), 6.35 (s, 1H), 6.25 (s, 1H), 5.87-5.96 (m, 1H), 3.55-3.63 (m, 1H), 3.06-3.16 (m, 1H), 2.90 (s, 3H), 2.15-2.28 (m, 1H), 1.48 (d, J=6.8 Hz, 3H).

### Example 42: (1*S,3*R)-7-(3-Chloro-2-fluoro-6-(1H-tetrazol-1-yl)phenyl)-1-methyl-3-(5-(2-methylpyridin-4-yl)-1H-imidazol-2-yl)-2,3-dihydroindolizin-5(1H)-one

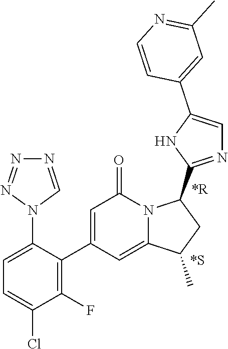 [SMILES: Cc1cc(-c2cnc([C@H]3C[C@H](C)c4cc(-c5c(-n6cnnn6)ccc(Cl)c5F)cc(=O)n43)[nH]2)ccn1]

LC/MS: mass calculated for C₂₅H₂₀ClFN₈O: 502.14, measured (ES, m/z): 503.10 [M+H]⁺. ¹H NMR (400 MHz, DMSO-d₆) δ12.33-12.97 (m, 1H), 9.66 (s, 1H), 8.36 (d, J=5.3 Hz, 1H), 8.02 (dd, J=8.7, 7.7 Hz, 1H), 7.80 (s, 1H), 7.74 (dd, J=8.7, 1.5 Hz, 1H), 7.58 (s, 1H), 7.49 (d, J=5.3 Hz, 1H), 6.11 (s, 1H), 6.04 (s, 1H), 5.52 (dd, J=8.7, 5.5 Hz, 1H), 3.33-3.40 (m, 1H), 2.71-2.83 (m, 1H), 2.49 (s, 3H), 1.96-2.04 (m, 1H), 1.32 (d, J=7.1 Hz, 3H). ¹⁹F NMR (376 MHz, DMSO-d₆) δ −73.40, −113.07.

### Example 43: (1*R,3*R)-7-(3-Chloro-2-fluoro-6-(1H-tetrazol-1-yl)phenyl)-3-(5-(2-fluoropyridin-4-yl)-1H-imidazol-2-yl)-1-methyl-2,3-dihydroindolizin-5(1H)-one

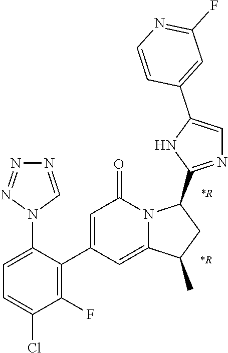 [SMILES: C[C@@H]1C[C@H](c2ncc(-c3ccnc(F)c3)[nH]2)n2c1cc(-c1c(-n3cnnn3)ccc(Cl)c1F)cc2=O]

LC/MS: mass calculated for C₂₄H₁₇ClF₂N₈O: 506.12, measured (ES, m/z): 507.20 [M+H]^{+ 1}H NMR (400 MHz, Methanol-d₄) δ 9.47 (s, 1H), 8.38 (d, J=5.3 Hz, 1H), 8.30 (s, 1H), 7.91 (dd, J=8.7, 7.6 Hz, 1H), 7.71-7.74 (m, 1H), 7.62 (dd, J=8.7, 1.6 Hz, 1H), 7.53 (s, 1H), 6.33 (s, 1H), 6.26 (s, 1H), 5.82-5.89 (m, 1H), 3.50-3.62 (m, 1H), 3.05-3.15 (m, 1H), 2.04-2.21 (m, 1H), 1.47 (d, J=6.8 Hz, 3H).

### Example 44: (1*S,3*S)-7-(3-Chloro-2-fluoro-6-(1H-tetrazol-1-yl)phenyl)-3-(5-(2-fluoropyridin-4-yl)-1H-imidazol-2-yl)-1-methyl-2,3-dihydroindolizin-5(1H)-one

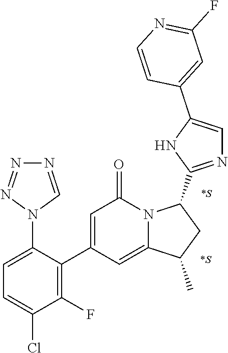 [SMILES: C[C@H]1C[C@@H](c2ncc(-c3ccnc(F)c3)[nH]2)n2c1cc(-c1c(-n3cnnn3)ccc(Cl)c1F)cc2=O]

LC/MS: mass calculated for C₂₄H₁₇ClF₂N₈O: 506.12, measured (ES, m/z): 507.20 [M+H]^{+ 1}H NMR (400 MHz, Methanol-d₄) δ 9.47 (s, 1H), 8.38 (d, J=5.3 Hz, 1H), 8.29 (s, 1H), 7.91 (dd, J=8.7, 7.6 Hz, 1H), 7.71-7.74 (m, 1H), 7.61 (dd, J=8.7, 1.6 Hz, 1H), 7.52 (s, 1H), 6.33 (s, 1H), 6.26 (s, 1H), 5.82-5.89 (m, 1H), 3.52-3.63 (m, 1H), 3.05-3.14 (m, 1H), 2.04-2.20 (m, 1H), 1.47 (d, J=6.8 Hz, 3H).

### Example 45: (1*R,3*S)-7-(3-Chloro-2-fluoro-6-(1H-tetrazol-1-yl)phenyl)-3-(5-(2-fluoropyridin-4-yl)-1H-imidazol-2-yl)-1-methyl-2,3-dihydroindolizin-5(1H)-one

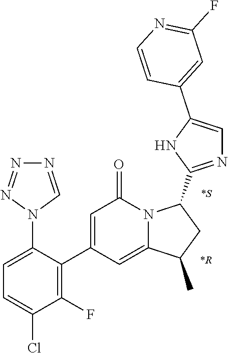 [SMILES: C[C@@H]1C[C@@H](c2ncc(-c3ccnc(F)c3)[nH]2)n2c1cc(-c1c(-n3cnnn3)ccc(Cl)c1F)cc2=O]

LC/MS: mass calculated for C₂₄H₁₇ClF₂N₈O: 506.12, measured (ES, m/z): 507.20 [M+H]^{+ 1}H NMR (400 MHz, Methanol-d₄) δ 9.46 (s, 1H), 8.38 (d, J=5.3 Hz, 1H), 8.29 (s, 1H), 7.91 (dd, J=8.7, 7.6 Hz, 1H), 7.71-7.74 (m, 1H), 7.61 (dd, J=8.7, 1.7 Hz, 1H), 7.52 (s, 1H), 6.33 (s, 1H), 6.26 (s, 1H), 5.82-5.88 (m, 1H), 3.49-3.63 (m, 1H), 3.03-3.13 (m, 1H), 2.04-2.20 (m, 1H), 1.47 (d, J=6.8 Hz, 3H).

### Example 46: (1*S,3*S)-7-(3-Chloro-2-fluoro-6-(1H-tetrazol-1-yl)phenyl)-1-methyl-3-(5-(2-methyl-2H-indazol-5-yl)-1H-imidazol-2-yl)-2,3-dihydroindolizin-5(1H)-one

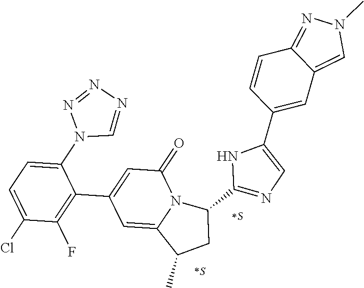 [SMILES: C[C@H]1C[C@@H](c2ncc(-c3ccc4nn(C)cc4c3)[nH]2)n2c1cc(-c1c(-n3cnnn3)ccc(Cl)c1F)cc2=O]

LC/MS: mass calculated for C₂₇H₂₁ClFN₉O: 541.15, measured (ES, m/z): 542.30 [M+H]⁺. ¹H NMR (400 MHz, DMSO-d₆): δ 14.04-15.20 (m, 1H), 9.67 (s, 1H), 8.50 (s, 1H), 8.10 (s, 1H), 7.92-8.08 (m, 1H), 7.68-7.80 (m, 2H), 7.60 (dd, J=9.0, 1.7 Hz, 1H), 6.22 (s, 1H), 6.14 (s, 1H), 5.67 (t, J=8.5 Hz, 1H), 4.19 (s, 3H), 3.37-3.50 (m, 1H), 2.81-2.92 (m, 1H), 1.95-2.06 (m, 1H), 1.34 (d, J=6.8 Hz, 3H). ¹⁹F NMR (376 MHz, DMSO-d₆): δ−73.81, −112.72.

### Example 47: (1*R,3*R)-7-(3-Chloro-2-fluoro-6-(1H-tetrazol-1-yl)phenyl)-1-methyl-3-(5-(2-methyl-2H-indazol-5-yl)-1H-imidazol-2-yl)-2,3-dihydroindolizin-5(1H)-one

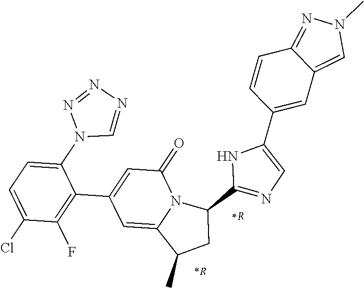 [SMILES: C[C@@H]1C[C@H](c2ncc(-c3ccc4nn(C)cc4c3)[nH]2)n2c1cc(-c1c(-n3cnnn3)ccc(Cl)c1F)cc2=O]

LC/MS: mass calculated for C₂₇H₂₁ClFN₉O: 541.15, measured (ES, m/z): 542.30 [M+H]⁺. ¹H NMR (400 MHz, DMSO-d₆): δ 13.97-15.18 (m, 1H), 9.67 (s, 1H), 8.50 (s, 1H), 8.11 (s, 1H), 7.93-8.08 (m, 2H), 7.69-7.79 (m, 2H), 7.60 (dd, J=9.0, 1.7 Hz, 1H), 6.22 (s, 1H), 6.14 (s, 1H), 5.67 (t, J=8.5 Hz, 1H), 4.19 (s, 3H), 3.37-3.53 (m, 1H), 2.80-2.95 (m, 1H), 1.93-2.07 (m, 1H), 1.34 (d, J=6.8 Hz, 3H). ¹⁹F NMR (376 MHz, DMSO-d₆): δ−73.80, −112.72.

### Example 48: (1S*,3S*)-7-(3-Chloro-2-fluoro-6-(1H-tetrazol-1-yl)phenyl)-3-(5-(imidazo[1,2-a]pyridin-6-yl)-1H-imidazol-2-yl)-1-methyl-2,3-dihydroindolizin-5(1H)-one

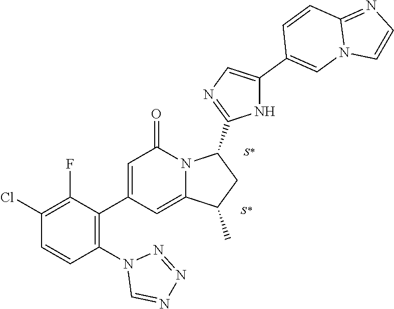 [SMILES: C[C@H]1C[C@@H](c2ncc(-c3ccc4nccn4c3)[nH]2)n2c1cc(-c1c(-n3cnnn3)ccc(Cl)c1F)cc2=O]

LC/MS: mass calculated for C₂₆H₁₉ClFN₉O: 527.14, measured (ES, m/z): 528.20 [M+H]⁺. ¹H NMR (300 MHz, DMSO-d₆) δ 12.23 (s, 1H), 9.66 (s, 1H), 8.78-8.82 (m, 1H), 7.94-8.05 (m, 2H), 7.70-7.80 (m, 1H), 7.48-7.61 (m, 4H), 6.09 (s, 1H), 6.03 (s, 1H), 5.45-5.60 (m, 1H), 3.30-3.41 (m, 1H), 2.70-2.85 (m, 1H), 1.95-2.10 (m, 1H), 1.30 (d, J=7.0 Hz, 3H). ¹⁹F NMR (282 MHz, DMSO-d₆) δ −113.12.

### Example 49: (1R*,3R*)-7-(3-Chloro-2-fluoro-6-(1H-tetrazol-1-yl)phenyl)-3-(5-(imidazo[1,2-a]pyridin-6-yl)-1H-imidazol-2-yl)-1-methyl-2,3-dihydroindolizin-5(1H)-one

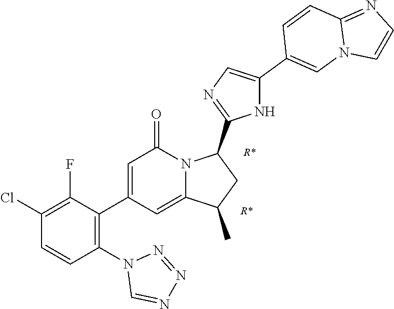 [SMILES: C[C@@H]1C[C@H](c2ncc(-c3ccc4nccn4c3)[nH]2)n2c1cc(-c1c(-n3cnnn3)ccc(Cl)c1F)cc2=O]

LC/MS: mass calculated for C₂₆H₁₉ClFN₉O: 527.14, measured (ES, m/z): 528.25 [M+H]⁺. ¹H NMR (300 MHz, DMSO-d₆) δ 12.23 (s, 1H), 9.66 (s, 1H), 8.75-8.90 (m, 1H), 7.94-8.05 (m, 2H), 7.70-7.80 (m, 1H), 7.48-7.61 (m, 4H), 6.09 (s, 1H), 6.03 (s, 1H), 5.45-5.55 (m, 1H), 3.30-3.41 (m, 1H), 2.72-2.90 (m, 1H), 1.90-2.10 (m, 1H), 1.30 (d, J=7.0 Hz, 3H). ¹⁹F NMR (282 MHz, DMSO-d₆) δ −113.12.

### Example 50: (1R,3S)-3-(5-(6-Amino-2-methylpyridin-3-yl)-1H-imidazol-2-yl)-7-(3-chloro-2-fluoro-6-(1H-tetrazol-1-yl)phenyl)-1-methyl-2,3-dihydroindolizin-5(1H)-one

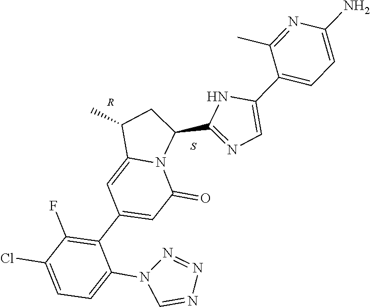

LC/MS: mass calculated for C₂₅H₂₁ClFN₉O: 517.15, measured (ES, m/z): 518.10 [M+H]⁺. ¹H NMR (400 MHz, Methanol-d₄) δ 9.38 (s, 1H), 7.85-7.93 (m, 1H), 7.79 (s, 1H), 7.60 (dd, J=8.7, 1.7 Hz, 1H), 7.12 (s, 1H), 6.66 (d, J=8.8 Hz, 1H), 6.30 (s, 1H), 6.10 (s, 1H), 5.77-5.84 (m, 1H), 3.77 (s, 1H), 2.63-2.72 (m, 1H), 2.48 (s, 3H), 2.20-2.32 (m, 1H), 1.28-1.34 (m, 3H). ¹⁹F NMR (376 MHz, Methanol-d₄) δ −76.94, −113.89.

### Example 51: (1*S,3*S)-3-(5-(6-Amino-2-methylpyridin-3-yl)-1H-imidazol-2-yl)-7-(3-chloro-2-fluoro-6-(1H-tetrazol-1-yl)phenyl)-1-methyl-2,3-dihydroindolizin-5(1H)-one

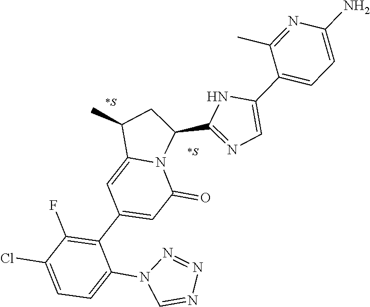

LC/MS: mass calculated for C₂₅H₂₁ClFN₉O: 517.15, measured (ES, m/z): 518.10 [M+H]⁺. ¹H NMR (400 MHz, Methanol-d₄) δ 9.38 (s, 1H), 8.12 (d, J=9.1 Hz, 1H), 7.89 (dd, J=8.7, 7.6 Hz, 1H), 7.60 (dd, J=8.7, 1.7 Hz, 1H), 7.30 (s, 1H), 6.90-6.94 (m, 1H), 6.23 (s, 1H), 6.18 (s, 1H), 5.61-5.69 (m, 1H), 3.46 (q, J=7.5 Hz, 1H), 2.84-2.96 (m, 1H), 2.60 (s, 3H), 2.10-2.22 (m, 1H), 1.40 (d, J=7.0 Hz, 3H). ¹⁹F NMR (376 MHz, Methanol-d₄) δ −76.95, −113.95.

### Example 52: (1*R,3*R)-7-(3-Chloro-2-fluoro-6-(1H-tetrazol-1-yl)phenyl)-1-methyl-3-(5-(pyrazolo[1,5-a]pyridin-5-yl)-1H-imidazol-2-yl)-2,3-dihydroindolizin-5(1H)-one

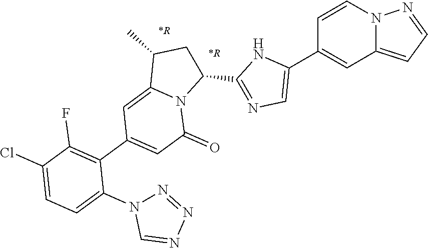

LC/MS: mass calculated for C₂₆H₁₉ClFN₉O: 527.14, measured (ES, m/z): 528.10 [M+H]⁺. ¹H NMR (300 MHz, DMSO-d₆) δ12.26-12.35 (m, 1H), 9.65 (s, 1H), 8.57 (d, J=7.4 Hz, 1H), 7.94-8.05 (m, 1H), 7.86-7.93 (m, 2H), 7.65-7.76 (m, 2H), 7.21 (d, J=7.4 Hz, 1H), 6.51-6.58 (m, 1H), 6.09 (s, 1H), 6.03 (s, 1H), 5.51 (dd, J=8.8, 5.2 Hz, 1H), 2.67-2.83 (m, 1H), 1.93-2.09 (m, 1H), 1.33 (d, J=7.2 Hz, 3H), 1.22 (s, 1H). ¹⁹F NMR (282 MHz, DMSO-d₆) δ−73.40, −113.05.

### Example 53: (1*S,3*S)-7-(3-Chloro-2-fluoro-6-(1H-tetrazol-1-yl)phenyl)-1-methyl-3-(5-(pyrazolo[1,5-a]pyridin-5-yl)-1H-imidazol-2-yl)-2,3-dihydroindolizin-5(1H)-one

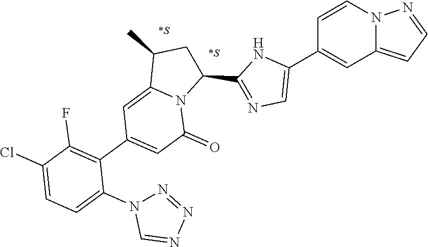

LC/MS: mass calculated for C₂₆H₁₉ClFN₉O: 527.14, measured (ES, m/z): 528.10 [M+H]⁺. ¹H NMR (300 MHz, DMSO-d₆) δ12.26-12.37 (m, 1H), 9.65 (s, 1H), 8.57 (d, J=7.3 Hz, 1H), 8.05-7.94 (m, 1H), 7.87-7.93 (m, 2H), 7.65-7.76 (m, 2H), 7.21 (d, J=7.5 Hz, 1H), 6.55 (s, 1H), 6.09 (s, 1H), 6.03 (s, 1H), 5.51 (dd, J=8.8, 5.1 Hz, 1H), 2.70-2.80 (m, 1H), 1.95-2.09 (m, 1H), 1.33 (d, J=7.1 Hz, 3H), 1.22 (s, 1H). ¹⁹F NMR (282 MHz, DMSO-d₆) δ−113.05.

### Example 54: (1 S*,3S*)-3-(2-(6-Amino-2-fluoropyridin-3-yl)-1H-imidazol-5-yl)-7-(3-chloro-2-fluoro-6-(1H-tetrazol-1-yl)phenyl)-1-methyl-2,3-dihydroindolizin-5(1H)-one

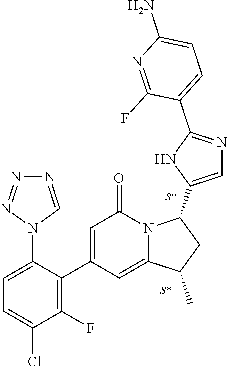 [SMILES: C[C@H]1C[C@@H](c2cnc(-c3ccc(N)nc3F)[nH]2)n2c1cc(-c1c(-n3cnnn3)ccc(Cl)c1F)cc2=O]

LC/MS: mass calculated for C₂₄H₁₈ClF₂N₉O: 521.13, measured (ES, m/z): 522.20 [M+H]⁺. ¹H NMR (400 MHz, DMSO-d₆) δ 9.67 (s, 1H), 7.93-8.06 (m, 2H), 7.70-7.77 (m, 1H), 6.58-6.96 (m, 3H), 6.44 (dd, J=8.4, 2.1 Hz, 1H), 6.11 (s, 1H), 5.98 (s, 1H), 5.50-5.60 (m, 1H), 2.62-2.78 (m, 1H), 1.96-2.04 (m, 1H), 1.16-1.32 (m, 4H). ¹⁹F NMR (376 MHz, DMSO-d₆) δ −71.33, −113.27.

### Example 55: (1R*,3R*)-3-(2-(6-Amino-2-fluoropyridin-3-yl)-1H-imidazol-5-yl)-7-(3-chloro-2-fluoro-6-(1H-tetrazol-1-yl)phenyl)-1-methyl-2,3-dihydroindolizin-5(1H)-one

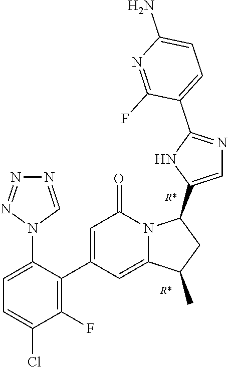 [SMILES: C[C@@H]1C[C@H](c2cnc(-c3ccc(N)nc3F)[nH]2)n2c1cc(-c1c(-n3cnnn3)ccc(Cl)c1F)cc2=O]

### Step 1: 7-(3-Chloro-2-fluoro-6-(H-tetrazol-1-yl)phenyl)-3-(2-chloroacetyl)-1-methylene-2,3-dihydroindolizin-5(1H)-one

To a cooled (0° C.) dichloromethane (10 mL) solution of 7-(3-chloro-2-fluoro-6-(1H-tetrazol-1-yl)phenyl)-1-methylene-5-oxo-1,2,3,5-tetrahydroindolizine-3-carboxylic acid (1 g, 2.57 mmol, 1.00 equiv.) was added a solution of oxalyl chloride (655 mg, 5.18 mmol, 2.00 equiv.) in dichloromethane (5 mL). One drop of N,N-dimethylformamide was added and the mixture was stirred at room temperature for 1 h. The reaction mixture was concentrated and the residue was dissolved in acetonitrile (15 mL). To the cooled (0° C.) acetonitrile solution, a 2 M solution of (trimethylsilyl) diazomethane in hexanes (3.8 mL, 7.737 mmol, 3.00 equiv.) was added and the mixture stirred at 0° C. for 1 h. To the reaction mixture, a solution of HCl (282 mg, 7.73 mmol, 3.00 equiv. 12 M in H₂O) was added and the mixture stirred at 0° C. for another 20 minutes. The reaction was quenched with H₂O (10 mL). The resulting mixture was extracted with ethyl acetate (3×10 mL). The organic layers were combined, dried over Na₂SO₄, filtered and concentrated to yield 7-(3-chloro-2-fluoro-6-(1H-tetrazol-1-yl)phenyl)-3-(2-chloroacetyl)-1-methylene-2,3-dihydroindolizin-5(1H)-one as a dark brown solid.

### Step 2: Tert-butyl (5-(5-(7-(3-chloro-2-fluoro-6-(1H-tetrazol-1-yl)phenyl)-1-methylene-5-oxo-1,2,3,5-tetrahydroindolizin-3-yl)-1H-imidazol-2-yl)-6-fluoropyridin-2-yl)carbamate

To a solution of 7-(3-chloro-2-fluoro-6-(1H-tetrazol-1-yl)phenyl)-3-(2-chloroacetyl)-1-methylene-2,3-dihydroindolizin-5(1H)-one (800 mg, 1.90 mmol, 1.00 equiv.) in THF/H₂O (12 mL) was added NaHCO₃ (480 mg, 5.71 mmol, 3.00 equiv.) and tert-butyl 5-carbamimidoyl-6-fluoropyridin-2-ylcarbamate (484 mg, 1.90 mmol, 1.00 equiv.). The mixture stirred at 75° C. for overnight. The reaction concentrated and purified by silica gel chromatography (0→5% CH₃OH/CH₂Cl₂) to yield tert-butyl 5-(5-(7-(3-chloro-2-fluoro-6-(1H-tetrazol-1-yl)phenyl)-1-methylene-5-oxo-1,2,3,5-tetrahydroindolizin-3-yl)-1H-imidazol-2-yl)-6-fluoropyridin-2-ylcarbamate as a brown solid.

### Step 3: 3-(2-(6-amino-2-fluoropyridin-3-yl)-1H-imidazol-5-yl)-7-(3-chloro-2-fluoro-6-(1H-tetrazol-1-yl)phenyl)-1-methylene-2,3-dihydroindolizin-5(1H)-one

To a solution of tert-butyl 5-(5-(7-(3-chloro-2-fluoro-6-(1H-tetrazol-1-yl)phenyl)-1-methylene-5-oxo-1,2,3,5-tetrahydroindolizin-3-yl)-1H-imidazol-2-yl)-6-fluoropyridin-2-ylcarbamate (200.0 mg, 0.32 mmol, 1.00 equiv.) in DCM (4 mL) was added TFA (1 mL). The mixture was stirred at room temperature for 2 h. The reaction was quenched with H₂O (10 mL) and the resulting mixture was extracted with ethyl acetate (3×20 mL). The organic layers were combined, dried over Na₂SO₄, filtered and concentrated. The residue was purified by reverse phase chromatography C18 column (0-40% CH₃CN/H₂O) to yield 3-(2-(6-amino-2-fluoropyridin-3-yl)-1H-imidazol-5-yl)-7-(3-chloro-2-fluoro-6-(1H-tetrazol-1-yl)phenyl)-1-methylene-2,3-dihydroindolizin-5(1H)-one as a white solid.

### Step 4: (1R,3R)-3-(2-(6-amino-2-fluoropyridin-3-yl)-1H-imidazol-5-yl)-7-(3-chloro-2-fluoro-6-(1H-tetrazol-1-yl)phenyl)-1-methyl-2,3-dihydroindolizin-5(1H)-one

To a solution of (R*)-3-(2-(6-amino-2-fluoropyridin-3-yl)-1H-imidazol-5-yl)-7-(3-chloro-2-fluoro-6-(1H-tetrazol-1-yl)phenyl)-1-methylene-2,3-dihydroindolizin-5(1H)-one (40 mg, 0.077 mmol, 1.00 equiv.) in CH₃OH/chlorobenzene (4.8 mL) was added Pd/C (82 mg, 0.077 mmol, 1.00 equiv., 10% w). The mixture was stirred at room temperature. for 20 min under H₂ atmosphere. The reaction was concentrated. and purified by C18 column chromatography (0→40% CH₃CN/H₂O) to yield (1R,3R)-3-(2-(6-amino-2-fluoropyridin-3-yl)-1H-imidazol-5-yl)-7-(3-chloro-2-fluoro-6-(1H-tetrazol-1-yl)phenyl)-1-methyl-2,3-dihydroindolizin-5(1H)-one as a white solid.

LC/MS: mass calculated for C₂₄H₁₈ClF₂N₉O: 521.13, measured (ES, m/z): 522.20 [M+H]⁺. ¹H NMR (400 MHz, DMSO-d₆) δ 9.67 (s, 1H), 7.93-8.06 (m, 2H), 7.72-7.78 (m, 1H), 6.56-7.20 (s, 3H), 6.46 (d, J=8.5 Hz, 1H), 6.12 (s, 1H), 6.02 (s, 1H), 5.54-5.60 (m, 1H), 2.68-2.80 (m, 1H), 1.95-2.04 (m, 1H), 1.21-1.28 (m, 4H). ¹⁹F NMR (376 MHz, DMSO-d₆) δ −71.30, −113.21.

### Example 56: (1*R,3*R)-3-(5-(6-Amino-2-fluoropyridin-3-yl)-1H-imidazol-2-yl)-7-(3-chloro-2-fluoro-6-(1H-tetrazol-1-yl)phenyl)-1-methyl-2,3-dihydroindolizin-5(1H)-one

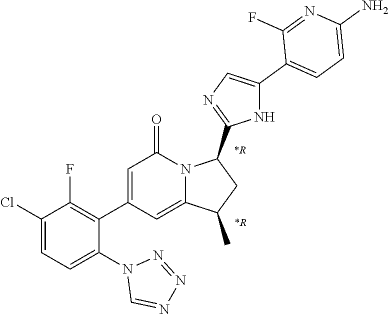 [SMILES: C[C@@H]1C[C@H](c2ncc(-c3ccc(N)nc3F)[nH]2)n2c1cc(-c1c(-n3cnnn3)ccc(Cl)c1F)cc2=O]

LC/MS: mass calculated for C₂₄H₁₈ClF₂N₉O: 521.13, measured (ES, m/z): 522.20 [M+H]⁺. ¹H NMR (400 MHz, DMSO-d₆): δ 9.57 (s, 1H), 7.84-7.99 (m, 2H), 7.65 (dd, J=8.7, 1.5 Hz, 1H), 7.34 (s, 1H), 6.44 (dd, J=8.3, 2.0 Hz, 1H), 6.09 (d, J=6.2 Hz, 2H), 5.56 (t, J=7.8 Hz, 1H), 3.29-3.38 (m, 1H), 2.72-2.84 (m, 1H), 1.87-1.99 (m, 1H), 1.27 (d, J=6.9 Hz, 3H). ¹⁹F NMR (376 MHz, DMSO-d₆): δ−73.57, −112.91.

### Example 57: (1*S,3*S)-3-(5-(6-Amino-2-chloropyridin-3-yl)-1H-imidazol-2-yl)-7-(3-chloro-2-fluoro-6-(1H-tetrazol-1-yl)phenyl)-1-methyl-2,3-dihydroindolizin-5(1H)-one

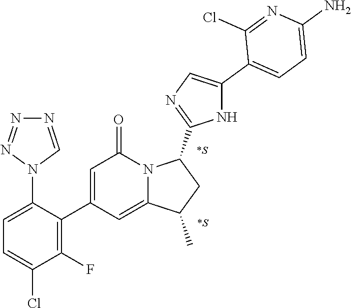 [SMILES: C[C@H]1C[C@@H](c2ncc(-c3ccc(N)nc3Cl)[nH]2)n2c1cc(-c1c(-n3cnnn3)ccc(Cl)c1F)cc2=O]

LC/MS: mass calculated for C₂₄H₁₈Cl₂FN₉O: 537.10, measured (ES, m/z): 538.20 [M+H]⁺. ¹H NMR (300 MHz, DMSO-d₆) δ 11.88-12.62 (m, 1H), 9.64 (s, 1H), 7.99 (dd, J=8.7, 7.7 Hz, 1H), 7.80-7.87 (m, 1H), 7.72 (dd, J=8.7, 1.6 Hz, 1H), 7.39 (s, 1H), 6.48 (d, J=8.5 Hz, 1H), 6.32-6.43 (m, 2H), 6.16 (s, 1H), 6.00 (s, 1H), 5.63 (d, J=8.6 Hz, 1H), 3.58-3.71 (m, 1H), 2.38-2.45 (m, 1H), 2.04-2.20 (m, 1H), 1.17 (d, J=6.8 Hz, 3H). ¹⁹F NMR (300 MHz, DMSO-d₆) δ −73.60, −113.08.

### Example 58: (1R,3S)-3-(5-(6-Amino-2-chloropyridin-3-yl)-1H-imidazol-2-yl)-7-(3-chloro-2-fluoro-6-(1H-tetrazol-1-yl)phenyl)-1-methyl-2,3-dihydroindolizin-5(1H)-one

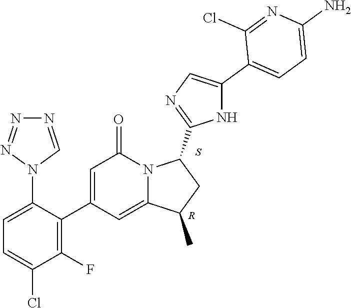

LC/MS: mass calculated for C₂₄H₁₈Cl₂FN₉O: 537.10, measured (ES, m/z): 538.15 [M+H]⁺. ¹H NMR (300 MHz, DMSO-d₆) δ 12.10-12.22 (m, 1H), 9.64 (s, 1H), 7.99 (dd, J=8.7, 7.7 Hz, 1H), 7.92 (d, J=8.4 Hz, 1H), 7.72 (dd, J=8.7, 1.5 Hz, 1H), 7.38 (d, J=2.1 Hz, 1H), 6.54-6.43 (m, 1H), 6.26-6.30 (m, 2H), 6.14 (s, 1H), 5.99 (s, 1H), 5.60 (d, J=8.5 Hz, 1H), 3.57-3.70 (m, 1H), 2.42-2.45 (m, 1H), 2.01-2.19 (m, 1H), 1.17 (d, J=6.8 Hz, 3H). ¹⁹F NMR (282 MHz, DMSO-d₆) δ −73.41, −113.12

### Example 59: (1S,3S)-3-(5-(6-Amino-2-chloropyridin-3-yl)-1H-imidazol-2-yl)-7-(3-chloro-2-fluoro-6-(1H-tetrazol-1-yl)phenyl)-1-methyl-2,3-dihydroindolizin-5(1H)-one

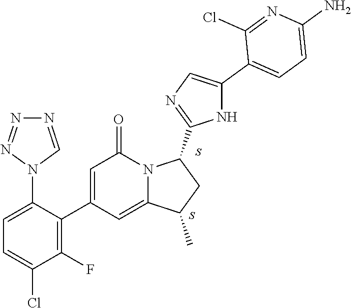

### Step 1: 2-(6-amino-2-chloropyridin-3-yl)-2-oxoethyl (1S,3S)-7-(3-chloro-2-fluoro-6-(1H-tetrazol-1-yl)phenyl)-1-methyl-5-oxo-1,2,3,5-tetrahydroindolizine-3-carboxylate

To a solution of (1S,3S)-7-(3-chloro-2-fluoro-6-(1H-tetrazol-1-yl)phenyl)-1-methyl-5-oxo-1,2,3,5-tetrahydroindolizine-3-carboxylic acid (0.26 g, 0.66 mmol, 1.0 equiv.) in DMF (8 mL) was added potassium carbonate (0.277 g, 2.00 mmol, 3 equiv). After the mixture was stirred for 30 min, 1-(6-amino-2-chloropyridin-3-yl)-2-bromoethan-1-one (0.250 g, 1.001 mmol, 1.5 equiv) in DMF (2 mL) was added. The reaction mixture was stirred for 2 h at room temperature, then diluted with water and extracted with ethyl acetate. The combined organic layer was dried over Na₂SO₄ and concentrated. The residue was purified by reverse column chromatography with CH₃CN/0.05% TFA water (5%-80%) to yield 2-(6-amino-2-chloropyridin-3-yl)-2-oxoethyl (1S,3S)-7-(3-chloro-2-fluoro-6-(1H-tetrazol-1-yl)phenyl)-1-methyl-5-oxo-1,2,3,5-tetrahydroindolizine-3-carboxylate as a light yellow solid.

### Step 2: (1S,3S)-3-(5-(6-amino-2-chloropyridin-3-yl)-1H-imidazol-2-yl)-7-(3-chloro-2-fluoro-6-(1H-tetrazol-1-yl)phenyl)-1-methyl-2,3-dihydroindolizin-5(1H)-one

To a solution of 2-(6-amino-2-chloropyridin-3-yl)-2-oxoethyl (1S,3S)-7-(3-chloro-2-fluoro-6-(1H-tetrazol-1-yl)phenyl)-1-methyl-5-oxo-1,2,3,5-tetrahydroindolizine-3-carboxylate (0.24 g, 0.43 mmol, 1.0 equiv.) in toluene (30 mL) was added ammonium acetate (0.49 g, 6.44 mmol, 15 equiv.), followed by addition of acetic acid (1 mL) under N₂. The reaction mixture was stirred for 2 h at 100° C., then cooled to room temperature and concentrated under vacuum. The residue was purified by reverse column chromatography with CH₃CN/0.05% TFA water (5%→80%) to yield the racemic mixture, which was purified by prep-Chiral-HPLC with (Hex:DCM=3:1)(0.1% DEA):EtOH=50:50 to yield (1S,3S)-3-(5-(6-amino-2-chloropyridin-3-yl)-1H-imidazol-2-yl)-7-(3-chloro-2-fluoro-6-(1H-tetrazol-1-yl)phenyl)-1-methyl-2,3-dihydroindolizin-5(1H)-one as a white solid.

LC/MS: mass calculated for C₂₄H₁₈Cl₂FN₉O: 537.10, measured (ES, m/z): 538.15 [M+H]^{+ 1}H NMR (300 MHz, DMSO-d₆) δ 12.17-12.26 (m, 1H), 9.63 (d, J=6.2 Hz, 1H), 7.90-8.08 (m, 2H), 7.71 (dd, J=8.7, 1.6 Hz, 1H), 7.39 (d, J=2.0 Hz, 1H), 6.48 (dd, J=8.2, 4.5 Hz, 1H), 6.30-6.34 (m, 2H), 6.08 (d, J=1.3 Hz, 1H), 5.99 (s, 1H), 5.51 (dd, J=8.8, 4.8 Hz, 1H), 2.65-2.80 (m, 1H), 2.49-2.51 (m, 1H), 1.98-2.01 (m, 1H), 1.19 (d, J=6.9 Hz, 3H). ¹⁹F NMR (282 MHz, DMSO-d₆) δ −73.41, −113.12.

### Example 60: (1*R)-3-(5-(6-Amino-5-fluoropyridin-3-yl)-1H-imidazol-2-yl)-7-(3-chloro-2-fluoro-6-(1H-tetrazol-1-yl)phenyl)-1-methyl-2,3-dihydroindolizin-5(1H)-one

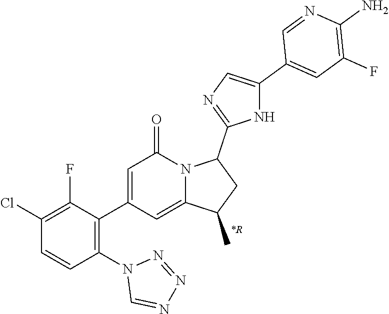

LC/MS: mass calculated for C₂₄H₁₈ClF₂N₉O: 521.13, measured (ES, m/z): 522.15 [M+H]⁺. ¹H NMR (300 MHz, DMSO-d₆) δ 12.06-12.33 (m, 1H), 9.63 (d, J=7.8 Hz, 1H), 8.04-8.16 (m, 1H), 7.99 (dd, J=8.7, 7.7 Hz, 1H), 7.71 (dd, J=8.7, 1.6 Hz, 1H), 7.54-7.61 (m, 1H), 7.37 (d, J=2.0 Hz, 1H), 5.95-6.11 (m, 4H), 5.40-5.51 (m, 1H), 3.19-3.23 (m, 1H), 2.63-2.80 (m, 1H), 1.85-2.09 (m, 1H), 1.32 (d, J=7.1 Hz, 3H). ¹⁹F NMR (282 MHz, DMSO-d₆) δ −73.41, −113.07, −139.94.

### Example 61: (1*S,3*S)-3-(5-(6-Amino-5-fluoropyridin-3-yl)-1H-imidazol-2-yl)-7-(3-chloro-2-fluoro-6-(1H-tetrazol-1-yl)phenyl)-1-methyl-2,3-dihydroindolizin-5(1H)-one

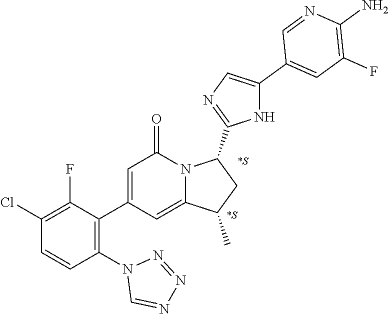

LC/MS: mass calculated for C₂₄H₁₈ClF₂N₉O: 521.13, measured (ES, m/z): 522.15 [M+H]^{+ 1}H NMR (300 MHz, DMSO-d₆) δ 12.07-12.47 (m, 1H), 9.64 (s, 1H), 8.10 (s, 1H), 7.99 (dd, J=8.7, 7.7 Hz, 1H), 7.71 (dd, J=8.7, 1.6 Hz, 1H), 7.54-7.60 (m, 1H), 7.37 (s, 1H), 6.13 (s, 1H), 6.06 (s, 2H), 6.03-6.09 (m, 1H), 5.57 (d, J=8.5 Hz, 1H), 3.63-3.76 (m, 1H), 2.46-2.41 (m, 1H), 2.19-2.01 (m, 1H), 1.16 (d, J=6.8 Hz, 3H). ¹⁹F NMR (282 MHz, DMSO-d₆) δ −73.40, −113.11, −139.96

### Example 62 (1*R,3*S)-3-(5-(6-amino-5-fluoropyridin-3-yl)-1H-imidazol-2-yl)-7-(3-chloro-2-fluoro-6-(1H-tetrazol-1-yl)phenyl)-1-methyl-2,3-dihydroindolizin-5(1H)-one

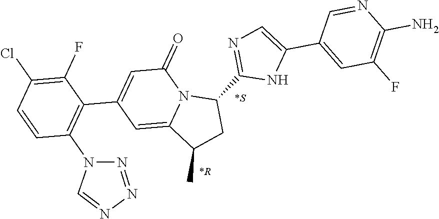 [SMILES: C[C@@H]1C[C@@H](c2ncc(-c3cnc(N)c(F)c3)[nH]2)n2c1cc(-c1c(-n3cnnn3)ccc(Cl)c1F)cc2=O]

### Step 1: 2-(6-amino-5-fluoropyridin-3-yl)-2-oxoethyl 7-(3-chloro-2-fluoro-6-(1H-tetrazol-1-yl)phenyl)-1-methyl-5-oxo-1,2,3,5-tetrahydroindolizine-3-carboxylate

To a solution of 7-(3-chloro-2-fluoro-6-(1H-tetrazol-1-yl)phenyl)-1-methyl-5-oxo-1,2,3,5-tetrahydroindolizine-3-carboxylic acid (300 mg, 0.77 mmol, 1.00 equiv.) in N,N-dimethylformamide (6 mL) was added potassium carbonate (319 mg, 2.30 mmol, 3.00 equiv.) and the mixture stirred at room temperature for 30 minutes. To the resulting mixture solution was then added 1-(6-amino-5-fluoropyridin-3-yl)-2-bromoethanone (608 mg, 1.539 mmol, 2.00 equiv.) in N,N-dimethylformamide (1.5 mL), and the mixture was stirred at room temperature for 2 hours. The reaction was quenched with H₂O (30 mL). The resulting mixture was extracted with ethyl acetate (3×30 mL). The organic layers were combined, dried over Na₂SO₄, filtered and concentrated. The residue was purified by silica gel chromatography (0-10% CH₃OH/CH₂Cl₂) to yield 2-(6-amino-5-fluoropyridin-3-yl)-2-oxoethyl 7-(3-chloro-2-fluoro-6-(1H-tetrazol-1-yl)phenyl)-1-methyl-5-oxo-1,2,3,5-tetrahydroindolizine-3-carboxylate as a light brown solid.

### Step 2: (1*R,3*S)-3-(5-(6-amino-5-fluoropyridin-3-yl)-1H-imidazol-2-yl)-7-(3-chloro-2-fluoro-6-(1H-tetrazol-1-yl)phenyl)-1-methyl-2,3-dihydroindolizin-5(1H)-one

To a solution of 2-(6-amino-5-fluoropyridin-3-yl)-2-oxoethyl 7-(3-chloro-2-fluoro-6-(1H-tetrazol-1-yl)phenyl)-1-methyl-5-oxo-1,2,3,5-tetrahydroindolizine-3-carboxylate (300 mg, 0.55 mmol, 1.00 equiv.) in toluene (6 mL) were added ammonium acetate (427 mg, 5.56 mmol, 10.00 equiv.) and acetic acid (0.6 mL, cat.). The resulting mixture was heated at reflux for 2 h. The reaction was quenched with H₂O (30 mL). The resulting mixture was extracted with ethyl acetate (3×30 mL). The organic layers were combined, dried over Na₂SO₄, filtered and concentrated. The residue was purified by C18 chromatography (0-50% CH₃CN/H₂O) to yield (3S*)-3-(5-(6-amino-5-fluoropyridin-3-yl)-1H-imidazol-2-yl)-7-(3-chloro-2-fluoro-6-(1H-tetrazol-1-yl)phenyl)-1-methyl-2,3-dihydroindolizin-5(1H)-one as a white solid and (3R*)-3-(5-(6-amino-5-fluoropyridin-3-yl)-1H-imidazol-2-yl)-7-(3-chloro-2-fluoro-6-(1H-tetrazol-1-yl)phenyl)-1-methyl-2,3-dihydroindolizin-5(1H)-one as a white solid. The racemic mixture was separated by Prep-Chiral-HPLC separation. The collected fractions were combined and concentrated under vacuum to yield (1R*,3S*)-3-(5-(6-amino-5-fluoropyridin-3-yl)-1H-imidazol-2-yl)-7-(3-chloro-2-fluoro-6-(1H-tetrazol-1-yl)phenyl)-1-methyl-2,3-dihydroindolizin-5(1H)-one as a white solid.

LC/MS: mass calculated for C₂₄H₁₈ClF₂N₉O: 521.13, measured (ES, m/z): 522.15 [M+H]⁺. ¹H NMR (300 MHz, DMSO-d₆) δ 12.06-12.38 (m, 1H), 9.64 (s, 1H), 8.10 (s, 1H), 7.99 (dd, J=8.7, 7.7 Hz, 1H), 7.71 (dd, J=8.7, 1.6 Hz, 1H), 7.53-7.61 (m, 1H), 7.31-7.39 (m, 1H), 6.13 (s, 1H), 6.03-6.10 (s, 2H), 5.97 (s, 1H), 5.57 (d, J=8.5 Hz, 1H), 3.63-3.76 (m, 1H), 2.41-2.46 (m, 1H), 2.01-2.19 (m, 1H), 1.16 (d, J=6.8 Hz, 3H). ¹⁹F NMR (282 MHz, DMSO-d₆) δ −73.41, −113.10, −139.95

### Example 63: Methyl(4-(2-((1S,3S)-7-(3-chloro-2-fluoro-6-(1H-tetrazol-1-yl)phenyl)-1-methyl-5-oxo-1,2,3,5-tetrahydroindolizin-3-yl)-1H-imidazol-5-yl)phenyl)carbamate

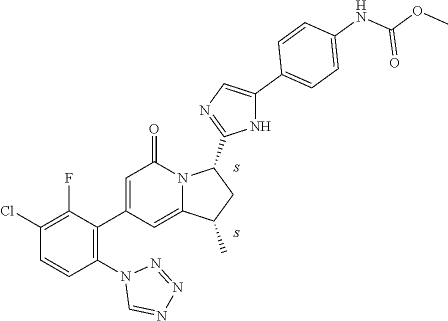

### Step 1: 2-(4-((Methoxycarbonyl)amino)phenyl)-2-oxoethyl 7-(3-chloro-2-fluoro-6-(1H-tetrazol-1-yl)phenyl)-1-methyl-5-oxo-1,2,3,5-tetrahydroindolizine-3-carboxylate

To a solution of (1 S,3S)-7-(3-chloro-2-fluoro-6-(1H-tetrazol-1-yl)phenyl)-1-methyl-5-oxo-1,2,3,5-tetrahydroindolizine-3-carboxylic acid (500 mg, 1.28 mmol, 1.00 equiv.) in N,N-dimethylformamide (7 mL) was added potassium carbonate (532 mg, 3.84 mmol, 3.00 equiv.) and the resulting mixture was stirred at room temperature for 30 minutes. To the resulting solution was then added methyl 4-(2-chloroacetyl)phenylcarbamate (380 mg, 1.68 mmol, 1.30 equiv.) in N,N-dimethylformamide (3 mL), and the mixture was stirred at room temperature for 2 hours, then quenched with H₂O (20 mL) and extracted with ethyl acetate (3×30 mL). The organic layers were combined, dried over Na₂SO₄, filtered and concentrated. The residue was purified by silica gel chromatography (0-100% EA/PE) to yield 2-(4-(methoxycarbonylamino)phenyl)-2-oxoethyl 7-(3-chloro-2-fluoro-6-(1H-tetrazol-1-yl)phenyl)-1-methyl-5-oxo-1,2,3,5-tetrahydroindolizine-3-carboxylate as a brown solid.

### Step 2: Methyl (4-(2-((1S,3S)-7-(3-chloro-2-fluoro-6-(1H-tetrazol-1-yl)phenyl)-1-methyl-5-oxo-1,2,3,5-tetrahydroindolizin-3-yl)-1H-imidazol-5-yl)phenyl)carbamate

To a solution of 2-(4-(methoxycarbonylamino)phenyl)-2-oxoethyl 7-(3-chloro-2-fluoro-6-(1H-tetrazol-1-yl)phenyl)-1-methyl-5-oxo-1,2,3,5-tetrahydroindolizine-3-carboxylate (500 mg, 0.86 mmol, 1.00 equiv.) in toluene (15 mL) were added ammonium acetate (663 mg, 8.60 mmol, 10.00 equiv.) and acetic acid (1.5 mL, cat.). The resulting mixture was heated at reflux for 2 h, then quenched with H₂O (30 mL) and extracted with ethyl acetate (3×30 mL). The organic layers were combined, dried over Na₂SO₄, filtered and concentrated. The residue was purified by C18 chromatography (0→50% CH₃CN/H₂O) to yield methyl 4-(2-((1S,3S)-7-(3-chloro-2-fluoro-6-(1H-tetrazol-1-yl)phenyl)-1-methyl-5-oxo-1,2,3,5-tetrahydroindolizin-3-yl)-1H-imidazol-5-yl)phenylcarbamate as a white solid.

LC/MS: mass calculated for C₂₇H₂₂ClFN₈O₃: 560.15, measured (ES, m/z): 561.05 [M+H]⁺. ¹H NMR (300 MHz, DMSO-d₆) δ 12.75-12.79 (m, 1H), 9.66 (s, 1H), 9.61 (s, 1H), 8.01 (t, J=8.2 Hz, 1H), 7.73 (dd, J=8.7, 1.5 Hz, 1H), 7.57-7.63 (m, 2H), 7.36-7.48 (m, 3H), 6.09 (s, 1H), 6.03 (s, 1H), 5.44-5.51 (m, 1H), 3.67 (s, 3H), 3.21-3.38 (m, 1H), 2.67-280 (m, 1H), 1.92-2.07 (m, 1H), 1.34 (d, J=7.1 Hz, 3H). ¹⁹F NMR (282 MHz, DMSO-d₆) δ −113.00.

### Example 64: Methyl(4-(2-((1*S,3*S)-7-(3-chloro-2-fluoro-6-(1H-tetrazol-1-yl)phenyl)-1-methyl-5-oxo-1,2,3,5-tetrahydroindolizin-3-yl)-1H-imidazol-5-yl)phenyl)carbamate

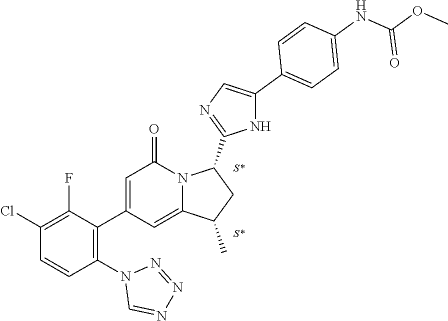

LC/MS: mass calculated for C₂₇H₂₂ClFN₈O₃: 560.15, measured (ES, m/z): 561.05 [M+H]⁺. ¹H NMR (300 MHz, DMSO-d₆) δ 12.04-12.48 (m, 1H), 9.64 (s, 1H), 9.55 (s, 1H), 7.99 (dd, J=8.7, 7.7 Hz, 1H), 7.71 (dd, J=8.7, 1.6 Hz, 1H), 7.57-7.60 (m, 1H), 7.29-7.56 (m, 4H), 6.14 (s, 1H), 5.98 (s, 1H), 5.58 (d, J=8.5 Hz, 1H), 3.67-3.78 (m, 1H), 3.64 (s, 3H), 2.24-2.45 (m, 1H), 1.96-2.17 (m, 1H), 1.17 (d, J=6.8 Hz, 3H). ¹⁹F NMR (282 MHz, DMSO-d₆) δ −113.07.

### Example 65: Methyl(4-(2-((1R,3S)-7-(3-chloro-2-fluoro-6-(1H-tetrazol-1-yl)phenyl)-1-methyl-5-oxo-1,2,3,5-tetrahydroindolizin-3-yl)-1H-imidazol-5-yl)phenyl)carbamate

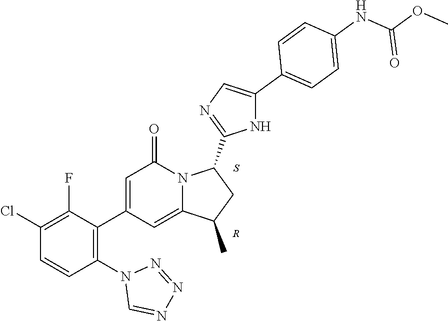

### Step 1: 2-(4-(Methoxycarbonylamino)phenyl)-2-oxoethyl 7-(3-chloro-2-fluoro-6-(1H-tetrazol-1-yl)phenyl)-1-methyl-5-oxo-1,2,3,5-tetrahydroindolizine-3-carboxylate

To a solution of 7-(3-chloro-2-fluoro-6-(1H-tetrazol-1-yl)phenyl)-1-methyl-5-oxo-1,2,3,5-tetrahydroindolizine-3-carboxylic acid (150 mg, 0.38 mmol, 1.00 equiv.) in N,N-dimethylformamide (2 mL) was added potassium carbonate (159 mg, 1.155 mmol, 3.00 equiv.) and the mixture was stirred at room temperature for 30 minutes. To the resulting solution was then added methyl 4-(2-chloroacetyl)phenylcarbamate (175 mg, 0.77 mmol, 2.00 equiv.) in N,N-dimethylformamide (1 mL), and the reaction mixture was stirred at room temperature for 2 h, quenched with H₂O (5 mL) and extracted with ethyl acetate (3×10 mL). The organic layers were combined, dried over Na₂SO₄, filtered and concentrated. The residue was purified by silica gel chromatography (0-100% EA/PE) to yield 2-(4-(methoxycarbonylamino)phenyl)-2-oxoethyl 7-(3-chloro-2-fluoro-6-(1H-tetrazol-1-yl)phenyl)-1-methyl-5-oxo-1,2,3,5-tetrahydroindolizine-3-carboxylate as a brown solid.

### Step 2: Methyl(4-(2-((1R,3S)-7-(3-chloro-2-fluoro-6-(1H-tetrazol-1-yl)phenyl)-1-methyl-5-oxo-1,2,3,5-tetrahydroindolizin-3-yl)-1H-imidazol-5-yl)phenyl)carbamate

To a solution of 2-(4-(methoxycarbonylamino)phenyl)-2-oxoethyl 7-(3-chloro-2-fluoro-6-(1H-tetrazol-1-yl)phenyl)-1-methyl-5-oxo-1,2,3,5-tetrahydroindolizine-3-carboxylate (100 mg, 0.17 mmol) in toluene (5 mL) was added ammonium acetate (132 mg, 1.72 mmol) and acetic acid (0.5 mL, cat.). The resulting mixture was heated at reflux for 2 h, quenched with H₂O (10 mL) and extracted with EtOAc (3×10 mL). The organic layers were combined, dried over Na₂SO₄, filtered and concentrated. The residue was purified by C18 chromatography (0-50% CH₃CN/H₂O) to yield methyl 4-(2-((3S*)-7-(3-chloro-2-fluoro-6-(1H-tetrazol-1-yl)phenyl)-1-methyl-5-oxo-1,2,3,5-tetrahydroindolizin-3-yl)-1H-imidazol-5-yl)phenylcarbamate and methyl 4-(2-((3R*)-7-(3-chloro-2-fluoro-6-(1H-tetrazol-1-yl)phenyl)-1-methyl-5-oxo-1,2,3,5-tetrahydroindolizin-3-yl)-1H-imidazol-5-yl)phenylcarbamate as a white solid. The racemic mixture was separated by prep-chiral-HPLC separation. The fractions were combined and concentrated under vacuum to yield methyl 4-(2-((1R*,3S*)-7-(3-chloro-2-fluoro-6-(1H-tetrazol-1-yl)phenyl)-1-methyl-5-oxo-1,2,3,5-tetrahydroindolizin-3-yl)-1H-imidazol-5-yl)phenylcarbamate as a white solid.

LC/MS: mass calculated for C₂₇H₂₂ClFN₈O₃: 560.15, measured (ES, m/z): 561.05 [M+H]⁺. ¹H NMR (300 MHz, DMSO-d₆) δ 12.04-12.48 (m, 1H), 9.64 (s, 1H), 9.55 (s, 1H), 7.93-8.05 (m, 1H), 7.71 (dd, J=8.7, 1.6 Hz, 1H), 7.33-7.63 (m, 5H), 6.10-6.15 (m, 1H), 5.98 (s, 1H), 5.58 (d, J=8.6 Hz, 1H), 3.67-3.77 (m, 1H), 3.64 (s, 3H), 2.41-2.45 (m, 1H), 2.19-2.02 (m, 1H), 1.17 (d, J=6.8 Hz, 3H). ¹⁹F NMR (282 MHz, DMSO-d₆) δ −113.07

### Example 66: (1R,3S)-3-(5-(6-Amino-2-fluoropyridin-3-yl)-1H-imidazol-2-yl)-7-(3-chloro-2-fluoro-6-(1H-tetrazol-1-yl)phenyl)-1-methyl-2,3-dihydroindolizin-5(1H)-one

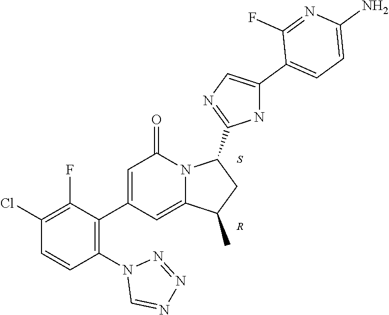

LC/MS: mass calculated for C₂₄H₁₈ClF₂N₉O: 521.13, measured (ES, m/z): 522.00 [M+H]⁺. ¹H NMR (300 MHz, DMSO-d₆) δ 12.07-12.25 (m, 1H), 9.65 (d, J=2.0 Hz, 1H), 8.05-7.86 (m, 2H), 7.72 (dd, J=8.7, 1.6 Hz, 1H), 7.05-7.08 (m, 1H), 6.36 (dd, J=8.3, 2.2 Hz, 1H), 6.20-6.24 (m, 2H), 6.14 (s, 1H), 5.99 (s, 1H), 5.59 (d, J=8.5 Hz, 1H), 3.60-3.73 (m, 1H), 2.39-2.43 (m, 1H), 2.05-2.19 (m, 1H), 1.17 (d, J=6.9 Hz, 3H). ¹⁹F NMR (282 MHz, DMSO-d₆) δ −70.52, −113.09.

### Example 67: (1S,3S)-3-(5-(6-Amino-2-fluoropyridin-3-yl)-1H-imidazol-2-yl)-7-(3-chloro-2-fluoro-6-(1H-tetrazol-1-yl)phenyl)-1-methyl-2,3-dihydroindolizin-5(1H)-one

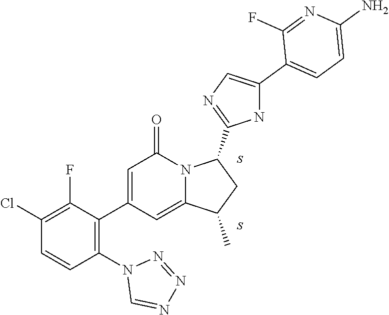

### Step 1: 4-(Benzyloxy)-2,6-dibromopyridine

To a solution of benzyl alcohol (17.2 mL, 166.3 mmol, 1.05 equiv.) in 1,4-dioxane (167 mL) was added sodium hydride (6.6 g, 166.3 mmol, 1.05 equiv. ca 60% dispersion in oil) at 0° C. The resulting mixture was stirred at room temperature for 1 h to yield the suspension of sodium phenyl methanolate. To a solution of 2,4,6-tribromopyridine (50.0 g, 158.3 mmol, 1.0 equiv.) in anhydrous N,N-dimethylformamide (833 mL) was added dropwise the above suspension of sodium phenyl methanolate at −20° C. The resulting mixture was warmed to room temperature slowly. After 2 h, the reaction was cooled to 0° C. and quenched with water (833 mL). The mixture was filtered. The filter cake was dissolved in petroleum ether (120 mL) and stirred for 30 min. Then the filter cake was collected, dried to yield the 4-(benzyloxy)-2,6-dibromopyridine as a white solid. LC/MS: mass calculated for C₁₂H₉Br₂NO: 340.91, measured (ES, m/z): 343.90 [M+H+2]⁺.

### Step 2: 4-(Benzyloxy)-2-bromo-6-((4-methoxybenzyl)oxy)pyridine

To a solution of (4-methoxyphenyl)methanol (17.6 g, 128.0 mmol, 1.05 equiv.) in 1,4-dioxane (400 mL) was added sodium hydride (60%, 5.3 g, 118.4 mmol, 1.1 equiv.) under nitrogen at 0° C. After 30 minutes, 4-(benzyloxy)-2,6-dibromopyridine (41.8 g, 121.9 mmol, 1.0 equiv.) was added. The resulting mixture was stirred 80° C. for 2 h. After cooling down to room temperature, to the reaction mixture was added water (200 mL), and the resulting mixture was extracted with ethyl acetate (3×200 mL). The organic layers were combined, dried over Na₂SO₄, filtered and concentrated to yield the 4-(benzyloxy)-2-bromo-6-((4-methoxybenzyl)oxy)pyridine as yellow oil. ¹H NMR (300 MHz, DMSO-d₆) δ 7.16-7.52 (m, 7H), 6.83-7.01 (m, 3H), 6.50 (d, J=1.9 Hz, 1H), 5.18 (d, J=4.9 Hz, 4H), 3.75 (s, 3H).

### Step 3: 4-(Benzyloxy)-6-bromopyridin-2-ol

To a solution of 4-(benzyloxy)-2-bromo-6-(4-methoxybenzyloxy)pyridine (60.5 g, 151.1 mmol, 1.0 equiv.) in dichloromethane (120 mL) was added trifluoroacetic acid (40 mL). The resulting mixture was stirred at room temperature for 3 hours. After most solvent was concentrated, sat. NaHCO₃ (aq., 100 mL) was added. The resulting mixture was stirred for 1 h. The solid was filtered and washed with water (200 mL) and petroleum ether (300 mL) to yield the 4-(benzyloxy)-6-bromopyridin-2-ol as white solid. LC/MS: mass calculated for C₁₂H₁₀BrNO₂: 278.99, measured (ES, m/z): 280.00[M+H]⁺.

### Step 4: Ethyl 2-(4-(benzyloxy)-6-bromo-2-oxopyridin-1(2H)-yl) acetate

To a solution of 4-(benzyloxy)-6-bromopyridin-2-ol (39 g, 140.4 mmol, 1.0 equiv.) in N,N-dimethylformamide (50 mL) and 1,2-dimethoxyethane (250 mL) was added sodium hydride (60%, 5.9 g, 147.4 mmol, 1.05 equiv.) under nitrogen at 0° C. After 1 h, lithium bromide (24.4 g, 280.8 mmol, 2.0 equiv.) was added. The resulting mixture was stirred at room temperature for 1 hour. The reaction mixture was cooled to 0° C. and was added ethyl 2-bromoacetate (31 mL, 280.8 mmol, 2.0 equiv.). The resulting mixture was heated to 65° C. for 2 h under nitrogen. After cooling down to 0° C., to the mixture was added ice water (500 mL). The mixture was filtered. The filter cake was added in a mixture solvents of ethyl acetate (75 mL) and petroleum ether (25 mL) and stirred at room temperature for 0.5 h. Then the solid was filtered, rinsed with water (50 mL) and dried under vacuum to yield ethyl 2-(4-(benzyloxy)-6-bromo-2-oxopyridin-1(2H)-yl) acetate as a white solid. LC/MS: mass calculated for C₁₆H₁₆BrNO₄: 365.03, measured (ES, m/z): 366.1[M+H]⁺.

### Step 5: Ethyl 2-(4-(benzyloxy)-6-bromo-2-oxopyridin-1(2H)-yl)pent-4-enoate

To a mixture of ethyl 2-(4-(benzyloxy)-6-bromo-2-oxopyridin-1(2H)-yl)acetate (20 g, 54.6 mmol, 1.0 equiv.) in THE (300 mL) at −70° C. under N₂ was added LiHMDS (64.5 mL, 65.5 mmol, 1.2 equiv.). After 1 hour, 3-iodoprop-1-ene (9.6 mL, 57.3 mmol, 1.05 equiv.) was added. The reaction was warmed to room temperature and stirred for 3 h. The reaction was quenched with saturated NH₄Cl solution and extracted with ethyl acetate (3×200 mL). The combined extracts were washed with water and brine, dried over anhydrous sodium sulfate, concentrated and purified by silica gel chromatography with ethyl acetate/petroleum ether (1→50%) to yield ethyl 2-(4-(benzyloxy)-6-bromo-2-oxopyridin-1(2H)-yl)pent-4-enoate as a yellow solid. LC/MS: mass calculated for C₁₉H₂₀BrNO₄: 405.06, measured (ES, m/z): 405.95 [M+H]⁺.

### Step 6: Ethyl 7-(benzyloxy)-1-methylene-5-oxo-1,2,3,5-tetrahydroindolizine-3-carboxylate

To a solution of ethyl 2-(4-(benzyloxy)-6-bromo-2-oxopyridin-1(2H)-yl)pent-4-enoate (20.0 g, 49.2 mmol, 1.0 equiv.) and triphenylphosphine (1.3 g, 4.9 mmol, 0.1 equiv.) in CH₃CN (230 mL) with diacetoxypalladium (552 mg, 2.5 mmol, 0.05 equiv.) was added triethylamine (13.7 mL, 98.5 mmol, 2.0 equiv.). The resulting mixture was stirred at 80° C. for 5 h, then concentrated and the residue was purified by silica gel chromatography with ethyl acetate/petroleum ether (1→80%), ethyl 7-(benzyloxy)-1-methylene-5-oxo-1,2,3,5-tetrahydroindolizine-3-carboxylate was obtained as a yellow solid. LC/MS: mass calculated for C₁₉H₁₉NO₄: 325.13, measured (ES, m/z): 326.10 [M+H]⁺.

### Step 7: Ethyl 7-hydroxy-1-methylene-5-oxo-1,2,3,5-tetrahydroindolizine-3-carboxylate

To a solution of ethyl 7-(benzyloxy)-1-methylene-5-oxo-1,2,3,5-tetrahydroindolizine-3-carboxylate (5.0 g, 15.37 mmol, 1.0 equiv.) in DCM (60 mL) was added BCl₃ (1M in dichloromethane) (30.7 mL, 30.7 mmol, 2.0 equiv.) at −70° C. The reaction was stirred for 4 h at room temperature. The reaction was quenched with H₂O (150 mL) and the resulting mixture was extracted with DCM (3×50 mL). The organic layers were combined, dried over Na₂SO₄, filtered and concentrated. The residue obtained was purified by silica gel chromatography (0→50% methanol/dichloromethane) to yield ethyl 7-hydroxy-1-methylene-5-oxo-1,2,3,5-tetrahydroindolizine-3-carboxylate a yellow solid. LC/MS: mass calculated for C₁₂H₁₃NO₄: 235.08, measured (ES, m/z): 236.05 [M+H]⁺.

### Step 8: Ethyl 1-methylene-5-oxo-7-(((trifluoromethyl)sulfonyl)oxy)-1,2,3,5-tetrahydroindolizine-3-carboxylate

To a mixture of ethyl 7-hydroxy-1-methylene-5-oxo-1,2,3,5-tetrahydroindolizine-3-carboxylate (2.0 g, 8.50 mmol, 1.0 equiv.) in DCM (30 mL) with triethylamine (2.4 mL, 17.0 mmol, 2.0 equiv.) was added trifluoro-N-phenyl-N-(trifluoromethylsulfonyl)methane sulfonamide (3.95 g, 11.05 mmol, 1.3 equiv.) at 0° C. The reaction was stirred at room temperature for 3 h. Water was added, and the mixture was extracted with DCM. The combined extracts were washed with water and brine and dried over anhydrous Na₂SO₄. The solvent was removed under vacuum and the residue was purified by silica gel chromatography with MeOH/DCM (1→5%) to yield ethyl 1-methylene-5-oxo-7-(((trifluoromethyl)sulfonyl)oxy)-1,2,3,5-tetrahydroindolizine-3-carboxylate as a yellow solid. LC/MS: mass calculated for C₁₃H₁₂F₃NO₆S: 367.03, measured (ES, m/z): 368.00 [M+H]⁺.

### Step 9: Ethyl 7-(6-amino-3-chloro-2-fluorophenyl)-1-methylene-5-oxo-1,2,3,5-tetrahydroindolizine-3-carboxylate

To a mixture of ethyl 1-methylene-5-oxo-7-(((trifluoromethyl)sulfonyl)oxy)-1,2,3,5-tetrahydroindolizine-3-carboxylate (2.0 g, 5.45 mmol, 1.0 equiv.) in 1,4-dioxane (30 mL), 6-amino-3-chloro-2-fluorophenylboronic acid (2.1 g, 10.89 mmol, 2.0 equiv.) and cesium fluoride (1.65 g, 10.89 mmol, 2.0 equiv.) was added tetrakis(triphenylphosphine) palladium (630 mg, 0.55 mmol, 0.1 equiv.). The reaction mixture was stirred at 100° C. for 3 h under N₂, diluted with water and extracted with ethyl acetate twice. The combined extracts were washed with water and brine, dried over anhydrous sodium sulfate and concentrated. The residue was purified by silica gel chromatography with methanol/dichloromethane (0→2%) to yield ethyl 7-(6-amino-3-chloro-2-fluorophenyl)-1-methylene-5-oxo-1,2,3,5-tetrahydroindolizine-3-carboxylate as a yellow solid. LC/MS: mass calculated for C₁₈H₁₆ClFN₂O₃: 362.08, measured (ES, m/z): 363.00 [M+H]⁺.

### Step 10: 7-(6-Amino-3-chloro-2-fluorophenyl)-1-methylene-5-oxo-1,2,3,5-tetrahydroindolizine-3-carboxylic acid

To a mixture of ethyl 7-(6-amino-3-chloro-2-fluorophenyl)-1-methylene-5-oxo-1,2,3,5-tetrahydroindolizine-3-carboxylate (1.6 g, 4.41 mmol, 1.0 equiv.) in ethanol (7 mL) and THE (20 mL) was added lithium hydroxide (10 mL, 2 M). The reaction was stirred at room temperature for 3 h. The mixture was adjusted to pH 5 with HCl solution (2 M) and extracted with ethyl acetate twice. The combined extracts were washed with water and saturated brine, dried over anhydrous sodium sulfate and concentrated to yield 7-(6-amino-3-chloro-2-fluorophenyl)-1-methylene-5-oxo-1,2,3,5-tetrahydroindolizine-3-carboxylic acid as an off-white solid. LC/MS: mass calculated for C₁₆H₁₂ClFN₂O₃: 334.05, measured (ES, m/z): 334.95 [M+H]⁺.

### Step 11: 7-(3-Chloro-2-fluoro-6-(1H-tetrazol-1-yl)phenyl)-1-methylene-5-oxo-1,2,3,5-tetrahydroindolizine-3-carboxylic acid

A mixture of 7-(6-amino-3-chloro-2-fluorophenyl)-1-methylene-5-oxo-1,2,3,5-tetrahydroindolizine-3-carboxylic acid (1.1 g, 3.29 mmol, 1.00 equiv.), azidotrimethylsilane (1.9 g, 16.43 mmol, 5.0 equiv.) and trimethoxymethane (3.5 g, 32.86 mmol, 10.0 equiv.) in glacial acetic acid (20 mL) was stirred overnight at room temperature. The mixture was concentrated under reduced pressure and the residue was purified by reverse phase chromatography on C18 column (CH₃CN/H₂O (0.05% CF₃COOH): 0→60%) to yield 7-(3-chloro-2-fluoro-6-(1H-tetrazol-1-yl)phenyl)-1-methylene-5-oxo-1,2,3,5-tetrahydroindolizine-3-carboxylic acid as an off-white solid. LC/MS: mass calculated for C₁₇H₁₁ClFN₅O₃: 387.05, measured (ES, m/z): 387.95 [M+H]⁺.

### Step 12: 2-(6-Amino-2-fluoropyridin-3-yl)-2-oxoethyl 7-(3-chloro-2-fluoro-6-(1H-tetrazol-1-yl)phenyl)-1-methylene-5-oxo-1,2,3,5-tetrahydroindolizine-3-carboxylate

To a solution of 7-(3-chloro-2-fluoro-6-(1H-tetrazol-1-yl)phenyl)-1-methylene-5-oxo-1,2,3,5-tetrahydroindolizine-3-carboxylic acid (150 mg, 0.39 mmol, 1.0 equiv.) in DMF (3 mL) was added K₂CO₃ (160 mg, 1.16 mmol, 3.0 equiv.), and the reaction mixture was stirred at room temperature for 30 min. 1-(6-Amino-2-fluoropyridin-3-yl)-2-bromoethan-1-one (135 mg, 0.58 mmol, 2.0 equiv.) was then added and the mixture was stirred for 2 h, then concentrated under vacuum. The residue was purified by reverse phase chromatography on C18 column (0→50% CH₃CN/H₂O) to yield 2-(6-amino-2-fluoropyridin-3-yl)-2-oxoethyl 7-(3-chloro-2-fluoro-6-(1H-tetrazol-1-yl)phenyl)-1-methylene-5-oxo-1,2,3,5-tetrahydroindolizine-3-carboxylate as a yellow solid. LC/MS: mass calculated for C₂₄H₁₆ClF₂N₇O₄: 539.09, measured (ES, m/z): 540.10 [M+H]⁺.

### Step 13: (S)-3-(5-(6-Amino-2-fluoropyridin-3-yl)-1H-imidazol-2-yl)-7-(3-chloro-2-fluoro-6-(1H-tetrazol-1-yl)phenyl)-1-methylene-2,3-dihydroindolizin-5(1H)-one

To a solution 2-(6-amino-2-fluoropyridin-3-yl)-2-oxoethyl 7-(3-chloro-2-fluoro-6-(1H-tetrazol-1-yl)phenyl)-1-methylene-5-oxo-1,2,3,5-tetrahydroindolizine-3-carboxylate (180 mg, 0.33 mmol, 1.0 equiv.) in toluene/acetic acid (5.5 mL, 10/1) was added NH₄OAc (257 mg, 3.33 mmol, 10.0 equiv.). The resulting mixture was stirred at 110° C. for 1 h, quenched with H₂O (10 mL) and extracted with ethyl acetate (3×20 mL). The organic layers were combined, dried over Na₂SO₄, filtered and concentrated. The residue was purified by reverse phase chromatography on C18 (0→50% CH₃CN/H₂O) and further purified by prep-chiral-HPLC to yield (S)-3-(5-(6-amino-2-fluoropyridin-3-yl)-1H-imidazol-2-yl)-7-(3-chloro-2-fluoro-6-(1H-tetrazol-1-yl)phenyl)-1-methylene-2,3-dihydroindolizin-5(1H)-one as a white solid. LC/MS: mass calculated for C₂₄H₁₆ClF₂N₉O: 519.11, measured (ES, m/z): 520.15 [M+H]⁺.

### Step 14: (1S,3S)-3-(5-(6-Amino-2-fluoropyridin-3-yl)-1H-imidazol-2-yl)-7-(3-chloro-2-fluoro-6-(1H-tetrazol-1-yl)phenyl)-1-methyl-2,3-dihydroindolizin-5(1H)-one

To a solution of (S)-3-(5-(6-amino-2-fluoropyridin-3-yl)-1H-imidazol-2-yl)-7-(3-chloro-2-fluoro-6-(1H-tetrazol-1-yl)phenyl)-1-methylene-2,3-dihydroindolizin-5(1H)-one (90 mg, 0.17 mmol, 1.0 equiv.) in MeOH (10 mL) and chlorobenzene (2 mL) was added 5% Pd/C (0.1 g). The reaction mixture was stirred for 20 min at room temperature under hydrogen atmosphere. Pd/C was filtered out and the resulting filtrate was evaporated under vacuum. The residue was purified by reverse column chromatography with CH₃CN/0.05% TFA water (5%→80%) to yield a residue, which was purified by Prep-Chiral-HPLC with MTBE (0.1% FA):EtOH=50/50 to yield (1 S,3S)-3-(5-(6-amino-2-fluoropyridin-3-yl)-1H-imidazol-2-yl)-7-(3-chloro-2-fluoro-6-(1H-tetrazol-1-yl)phenyl)-1-methyl-2,3-dihydroindolizin-5(1H)-one as a white solid.

LC/MS: mass calculated for C₂₄H₁₈ClF₂N₉O: 521.13, measured (ES, m/z): 522.20 [M+H]⁺. ¹H NMR (300 MHz, DMSO-d₆) δ 12.08-12.22 (m, 1H), 9.62 (s, 1H), 7.89-8.04 (m, 2H), 7.70 (dd, J=8.7, 1.6 Hz, 1H), 7.03-7.11 (m, 1H), 6.36 (dd, J=8.2, 2.2 Hz, 1H), 6.19-6.24 (m, 2H), 6.08 (s, 1H), 5.99 (s, 1H), 5.46-5.53 (m, 1H), 3.37-3.44 (m, 1H), 2.63-2.80 (m, 1H), 1.94-2.02 (m, 1H), 1.30 (d, J=7.1 Hz, 3H). ¹⁹F NMR (300 MHz, DMSO-d₆) δ −70.48, −113.04

### Example 68: (1*S,3*S)-3-(5-(6-Amino-2-fluoropyridin-3-yl)-1H-imidazol-2-yl)-7-(3-chloro-2-fluoro-6-(1H-tetrazol-1-yl)phenyl)-1-methyl-2,3-dihydroindolizin-5(1H)-one

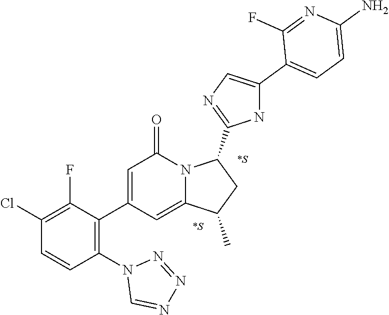

LC/MS: mass calculated for C₂₄H₁₈ClF₂N₉O: 521.13, measured (ES, m/z): 522.00 [M+H]⁺. ¹H NMR (300 MHz, DMSO-d₆) δ 12.06-12.24 (m, 1H), 9.65 (d, J=2.0 Hz, 1H), 7.99-7.90 (m, 2H), 7.72 (dd, J=8.7, 1.6 Hz, 1H), 7.06-7.08 (m, 1H), 6.36 (dd, J=8.2, 2.3 Hz, 1H), 6.21-6.24 (m, 2H), 6.13 (d, J=6.1 Hz, 1H), 5.98 (d, J=6.5 Hz, 1H), 5.59 (d, J=8.5 Hz, 1H), 3.61-3.71 (m, 1H), 2.39-2.42 (m, 1H), 2.01-2.19 (m, 1H), 1.17 (d, J=6.9 Hz, 3H). ¹⁹F NMR (282 MHz, DMSO-d₆) δ −70.52, −113.09.

### Example 69 2′-(3-Chloro-2-fluoro-6-(1H-tetrazol-1-yl)phenyl)-6′-(4-fluoro-5-(3-fluoro-2-(hydroxymethyl)pyridin-4-yl)-1H-imidazol-2-yl)-6′,7′-dihydro-4′H-spiro[cyclopropane-1,8′-pyrrolo[1,2-a]pyrimidin]-4′-one

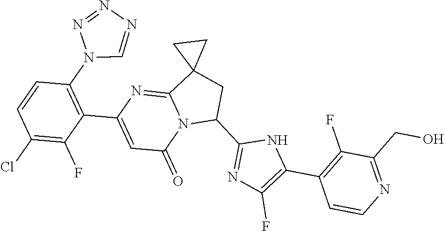 [SMILES: O=c1cc(-c2c(-n3cnnn3)ccc(Cl)c2F)nc2n1C(c1nc(F)c(-c3ccnc(CO)c3F)[nH]1)CC21CC1]

### Step 1: 2-(2-(((tert-butyldimethylsilyl)oxy)methyl)-3-fluoropyridin-4-yl)-2-oxoethyl 2′-(3-chloro-2-fluoro-6-(1H-tetrazol-1-yl)phenyl)-4′-oxo-6′,7′-dihydro-4′H-spiro[cyclopropane-1,8′-pyrrolo[1,2-a]pyrimidine]-6′-carboxylate

To a solution of 2′-(3-chloro-2-fluoro-6-(1H-tetrazol-1-yl)phenyl)-4′-oxo-6′,7′-dihydro-4′H-spiro[cyclopropane-1,8′-pyrrolo[1,2-a]pyrimidine]-6′-carboxylic acid (345 mg, 0.86 mmol, 1.0 equiv.) in DMF (15 mL) was added K₂CO₃ (236 mg, 1.71 mmol, 2.0 equiv.) at room temperature. After stirring for 30 min, 2-bromo-1-(2-(((tert-butyldimethylsilyl)oxy)methyl)-3-fluoropyridin-4-yl)ethan-1-one (620 mg, 1.71 mmol, 2.0 equiv.) was added. The resulting mixture was stirred at room temperature for 1.5 h, diluted with ethyl acetate and washed with water. The organic layers were combined, dried over anhydrous sodium sulfate, filtered and concentrated. The residue was purified by silica gel chromatography (0→80% ethyl acetate/petroleum ether) to yield the 2-(2-(((tert-butyldimethylsilyl)oxy)methyl)-3-fluoropyridin-4-yl)-2-oxoethyl 2′-(3-chloro-2-fluoro-6-(1H-tetrazol-1-yl)phenyl)-4′-oxo-6′,7′-dihydro-4′H-spiro[cyclopropane-1,8′-pyrrolo[1,2-a]pyrimidine]-6′-carboxylate as an yellow oil.

### Step 2: 6′-(5-(2-(((tert-butyldimethylsilyl)oxy)methyl)-3-fluoropyridin-4-yl)-1H-imidazol-2-yl)-2′-(3-chloro-2-fluoro-6-(1H-tetrazol-1-yl)phenyl)-6′,7′-dihydro-4′H-spiro[cyclopropane-1,8′-pyrrolo[1,2-a]pyrimidin]-4′-one

To a solution of 2-(3-fluoro-2-(hydroxymethyl)pyridin-4-yl)-2-oxoethyl 2′-(3-chloro-2-fluoro-6-(1H-tetrazol-1-yl)phenyl)-4′-oxo-6′,7′-dihydro-4′H-spiro[cyclopropane-1,8′-pyrrolo[1,2-a]pyrimidine]-6′-carboxylate (280 mg, 0.41 mmol, 1.0 equiv.) in toluene (10 mL) and HOAc (0.3 mL) was added NH₄OAc (631 mg, 8.19 mmol, 20.0 equiv.). The resulting mixture was stirred at 100° C. for 1 h. The resulting mixture was concentrated. The residue was purified by silica gel chromatography (0→10% MeOH/DCM) to yield 6′-(5-(2-(((tert-butyldimethylsilyl)oxy)methyl)-3-fluoropyridin-4-yl)-1H-imidazol-2-yl)-2′-(3-chloro-2-fluoro-6-(1H-tetrazol-1-yl)phenyl)-6′,7′-dihydro-4′H-spiro[cyclopropane-1,8′-pyrrolo[1,2-a]pyrimidin]-4′-one as an yellow solid.

### Step 3: 6′-(5-(2-(((tert-butyldimethylsilyl)oxy)methyl)-3-fluoropyridin-4-yl)-4-fluoro-1H-imidazol-2-yl)-2′-(3-chloro-2-fluoro-6-(1H-tetrazol-1-yl)phenyl)-6′,7′-dihydro-4′H-spiro[cyclopropane-1,8′-pyrrolo[1,2-a]pyrimidin]-4′-one

To a solution of 6′-(5-(2-(((tert-butyldimethylsilyl)oxy)methyl)-3-fluoropyridin-4-yl)-1H-imidazol-2-yl)-2′-(3-chloro-2-fluoro-6-(1H-tetrazol-1-yl)phenyl)-6′,7′-dihydro-4′H-spiro[cyclopropane-1,8′-pyrrolo[1,2-a]pyrimidin]-4′-one (70 mg, 0.11 mmol, 1.0 equiv.) in ACN (3 mL) and THE (1 mL) were added pyridine (83 mg, 1.05 mmol, 10.0 equiv.) and 1-chloromethyl-4-fluoro-1,4-diazoniabicyclo[2.2.2]octane bis(tetrafluoroborate) (93 mg, 0.26 mmol, 2.5 equiv.) at 0° C. The mixture was stirred at 0° C. for 2 h, quenched with brine and extracted with ethyl acetate. The organic layers were combined, dried over anhydrous sodium sulfate, filtered and concentrated. The residue was purified by silica gel chromatography (0-5% MeOH/DCM) to yield the 6′-(5-(2-(((tert-butyldimethylsilyl)oxy)methyl)-3-fluoropyridin-4-yl)-4-fluoro-1H-imidazol-2-yl)-2′-(3-chloro-2-fluoro-6-(1H-tetrazol-1-yl)phenyl)-6′,7′-dihydro-4′H-spiro[cyclopropane-1,8′-pyrrolo[1,2-a]pyrimidin]-4′-one as a red solid.

### Step 4: 2′-(3-chloro-2-fluoro-6-(1H-tetrazol-1-yl)phenyl)-6′-(4-fluoro-5-(3-fluoro-2-(hydroxymethyl)pyridin-4-yl)-1H-imidazol-2-yl)-6′,7′-dihydro-4′H-spiro[cyclopropane-1,8′-pyrrolo[1,2-a]pyrimidin]-4′-one

To a solution of 6′-(5-(2-(((tert-butyldimethylsilyl)oxy)methyl)-3-fluoropyridin-4-yl)-4-fluoro-1H-imidazol-2-yl)-2′-(3-chloro-2-fluoro-6-(1H-tetrazol-1-yl)phenyl)-6′,7′-dihydro-4′H-spiro[cyclopropane-1,8′-pyrrolo[1,2-a]pyrimidin]-4′-one (22 mg, 0.032 mmol) in THE (4 mL) was added triethylamine trihydrofluoride (0.5 mL). The resulting mixture was stirred at 70° C. for 2 h. After cooling to room temperature, the resulting mixture was concentrated and prep-HPLC (Column: YMC-Actus Triart C18 ExRS, 30 mm×150 mm, 5 um; Mobile Phase A: Water (10 mmol/L NH₄HCO₃), Mobile Phase B:ACN; Flow rate: 60 mL/min; Gradient: 28 B to 52 B in 8 min, 254/220 nm; room temperature 1:7.25; room temperature; to yield 2′-(3-chloro-2-fluoro-6-(1H-tetrazol-1-yl)phenyl)-6′-(4-fluoro-5-(3-fluoro-2-(hydroxymethyl)pyridin-4-yl)-1H-imidazol-2-yl)-6′,7′-dihydro-4′H-spiro[cyclopropane-1,8′-pyrrolo[1,2-a]pyrimidin]-4′-one as an yellow solid.

LC/MS mass calculated for: C₂₅H₁₇ClF₃N₉O₂: 567.92, measured (ES, m/z): 568.05[M+H]⁺. ¹H NMR (400 MHz, DMSO-d₆) δ 13.00 (s, 1H), 9.72 (s, 1H), 8.38 (d, J=5.1 Hz, 1H), 8.03 (dd, J=8.7, 7.7 Hz, 1H), 7.72 (dd, J=8.7, 1.5 Hz, 1H), 7.49 (t, J=5.4 Hz, 1H), 6.53 (d, J=2.3 Hz, 1H), 5.62-5.79 (m, 1H), 5.35 (t, J=6.0 Hz, 1H), 4.63 (dd, J=5.9, 2.3 Hz, 2H), 2.76 (dd, J=13.2, 9.7 Hz, 1H), 2.15 (dd, J=13.2, 3.8 Hz, 1H), 0.92-1.12 (m, 2H), 0.66-0.89 (m, 2H). ¹⁹F NMR (376 MHz, DMSO-d₆) δ −113.48, −125.53, −130.21.

### Example 70: (R*)-2′-(3-Chloro-2-fluoro-6-(1H-tetrazol-1-yl)phenyl)-6′-(5-(3-fluoro-2-(2-hydroxy-2-methylpropoxy)pyridin-4-yl)-1H-imidazol-2-yl)-6′,7′-dihydro-4′H-spiro[cyclopropane-1,8′-pyrrolo[1,2-a]pyrimidin]-4′-one

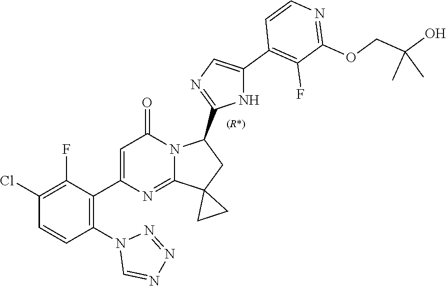 [SMILES: CC(C)(O)COc1nccc(-c2cnc([C@H]3CC4(CC4)c4nc(-c5c(-n6cnnn6)ccc(Cl)c5F)cc(=O)n43)[nH]2)c1F]

LC/MS: mass calculated for C₂₈H₂₄ClF₂N₉O₃: 607.17, measured: 608.10 [M+H]⁺. ¹H NMR (400 MHz, DMSO-d₆) δ 9.64 (s, 1H), 7.90-7.91 (m, 1H), 7.86 (d, J=5.4 Hz, 1H), 7.59-7.69 (m, 2H), 7.47-7.49 (m, 1H), 6.50 (d, J=2.3 Hz, 1H), 5.72 (dd, J=9.4, 3.7 Hz, 1H), 4.09 (s, 2H), 2.68-2.74 (m, 1H), 2.10-2.15 (m, 1H), 1.17 (s, 6H), 0.95-1.09 (m, 2H), 0.64-0.84 (m, 2H). ¹⁹F NMR (376 MHz, DMSO-d₆) δ−73.82, −113.63, −142.96.

### Example 71: Methyl-d₃ (R*)-(4-(2-(2′-(3-Chloro-2-fluoro-6-(1H-tetrazol-1-yl)phenyl)-4′-oxo-6′,7′-dihydro-4′H-spiro[cyclopropane-1,8′-pyrrolo[1,2-a]pyrimidin]-6′-yl)-1H-imidazol-5-yl)phenyl)carbamate

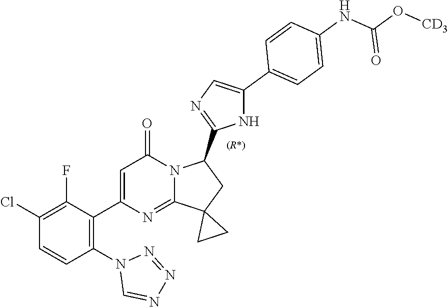 [SMILES: [2H]C([2H])([2H])OC(=O)Nc1ccc(-c2cnc([C@H]3CC4(CC4)c4nc(-c5c(-n6cnnn6)ccc(Cl)c5F)cc(=O)n43)[nH]2)cc1]

LC/MS: mass calculated for C₂₇H₁₈D3ClFN₉O₃: 576.16, measured: 577.15 [M+H]⁺. ¹H NMR (400 MHz, DMSO-d₆) δ 12.15-12.52 (m, 1H), 9.70 (d, J=6.6 Hz, 1H), 9.57 (s, 1H), 8.02 (t, J=8.2 Hz, 1H), 7.71 (d, J=8.7 Hz, 1H), 7.62 (d, J=8.4 Hz, 2H), 7.41-7.47 (m, 3H), 6.45-6.54 (m, 1H), 5.67 (dd, J=9.5, 3.4 Hz, 1H), 2.67-2.77 (m, 1H), 2.05-2.28 (m, 1H), 0.93-1.12 (m, 2H), 0.66-0.88 (m, 2H)

### Example 72: Methyl-d₃ (S*)-(4-(2-(2′-(3-Chloro-2-fluoro-6-(1H-tetrazol-1-yl)phenyl)-4′-oxo-6′,7′-dihydro-4′H-spiro[cyclopropane-1,8′-pyrrolo[1,2-a]pyrimidin]-6′-yl)-1H-imidazol-5-yl)phenyl)carbamate

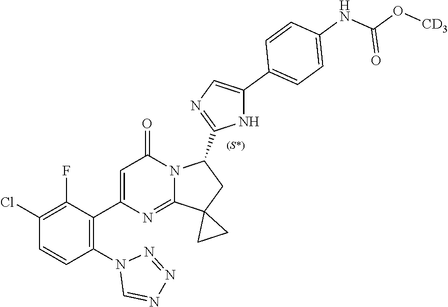 [SMILES: [2H]C([2H])([2H])OC(=O)Nc1ccc(-c2cnc([C@@H]3CC4(CC4)c4nc(-c5c(-n6cnnn6)ccc(Cl)c5F)cc(=O)n43)[nH]2)cc1]

LC/MS: mass calculated for C₂₇H₁₆D3ClFN₉O₃: 576.16, measured: 577.15 [M+H]⁺. ¹H NMR (400 MHz, DMSO-d₆) δ 12.13-12.55 (m, 1H), 9.60-9.80 (m, 1H), 9.58 (s, 1H), 7.99-8.08 (m, 1H), 7.67-7.77 (m, 1H), 7.56-7.63 (m, 2H), 7.37-7.50 (m, 3H), 6.50 (s, 1H), 5.65-5.72 (m, 1H), 2.68-2.81 (m, 1H), 2.17-2.30 (m, 1H), 0.96-1.12 (m, 2H), 0.60-0.95 (m, 2H).

### Example 73: (S)-2′-(3-chloro-2-fluoro-6-(1H-tetrazol-1-yl)phenyl)-6′-(4-(3-fluoro-2-((S*)-3,3,3-trifluoro-2-hydroxypropoxy)pyridin-4-yl)-1H-imidazol-2-yl)-6′,7′-dihydro-4′H-spiro[cyclopropane-1,8′-pyrrolo[1,2-a]pyrimidin]-4′-one

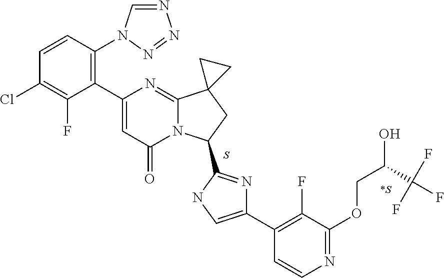

LC/MS: mass calculated for C₂₇H₁₉ClF₅N₉O₃: 647.12, measured: 648.05 [M+H]⁺. ¹H NMR (300 MHz, DMSO-d₆) δ 12.71 (s, 1H), 9.73 (m, 1H), 8.05 (t, J=8.7 Hz, 1H), 7.93 (d, J=5.3 Hz, 1H), 7.74 (d, J=8.6 Hz, 1H), 7.64-7.67 (m, 1H), 7.58-7.62 (m, 1H), 6.68 (d, J=6.2 Hz, 1H), 6.54 (d, J=2.3 Hz, 1H), 5.75 (dd, J=9.4, 3.3 Hz, 1H), 4.52-4.57 (m, 1H), 4.42-4.50 (m, 2H), 2.72-2.82 (m, 1H), 2.19-2.27 (m, 1H), 1.00-1.15 (m, 2H), 0.77-0.88 (m, 2H). ¹⁹F NMR (282 MHz, DMSO-d₆) δ −76.17, −113.62, −143.15.

### Example 74: (S)-2′-(3-chloro-2-fluoro-6-(1H-tetrazol-1-yl)phenyl)-6′-(4-(3-fluoro-2-((R*)-3,3,3-trifluoro-2-hydroxypropoxy)pyridin-4-yl)-1H-imidazol-2-yl)-6′,7′-dihydro-4′H-spiro[cyclopropane-1,8′-pyrrolo[1,2-a]pyrimidin]-4′-one

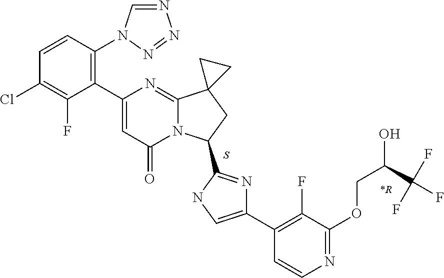

### Step 1: 2-(3-Fluoro-2-(3,3,3-trifluoro-2-hydroxypropoxy)pyridin-4-yl)-2-oxoethyl 2′-(3-chloro-2-fluoro-6-(1H-tetrazol-1-yl)phenyl)-4′-oxo-6′,7′-dihydro-4′H-spiro[cyclopropane-1,8′-pyrrolo[1,2-a]pyrimidine]-6′-carboxylate

To a solution of 2′-(3-chloro-2-fluoro-6-(1H-tetrazol-1-yl)phenyl)-4′-oxo-6′,7′-dihydro-4′H-spiro[cyclopropane-1,8′-pyrrolo[1,2-a]pyrimidine]-6′-carboxylic acid (200 mg, 0.49 mmol, 1.0 equiv) in DMF (5 mL) were added 2-bromo-1-(3-fluoro-2-(3,3,3-trifluoro-2-hydroxypropoxy)pyridin-4-yl)ethan-1-one (515 mg, 1.49 mmol, 3.0 equiv) and Cs₂CO₃ (97 mg, 0.29 mmol, 0.6 equiv). The reaction mixture was stirred overnight at room temperature, quenched with water and extracted with ethyl acetate. The combined organic layer was washed with brine, dried over Na₂SO₄ and concentrated under vacuum. The residue was purified by silica gel chromatography (0-30% MeOH/DCM) to yield 2-(3-fluoro-2-(3,3,3-trifluoro-2-hydroxypropoxy)pyridin-4-yl)-2-oxoethyl 2′-(3-chloro-2-fluoro-6-(1H-tetrazol-1-yl)phenyl)-4′-oxo-6′,7′-dihydro-4′H-spiro[cyclopropane-1,8′-pyrrolo[1,2-a]pyrimidine]-6′-carboxylate as a yellow solid.

### Step 2: (S)-2′-(3-chloro-2-fluoro-6-(1H-tetrazol-1-yl)phenyl)-6′-(4-(3-fluoro-2-((R*)-3,3,3-trifluoro-2-hydroxypropoxy)pyridin-4-yl)-1H-imidazol-2-yl)-6′,7′-dihydro-4′H-spiro[cyclopropane-1,8′-pyrrolo[1,2-a]pyrimidin]-4′-one

To a solution of 2-(3-fluoro-2-(3,3,3-trifluoro-2-hydroxypropoxy)pyridin-4-yl)-2-oxoethyl 2′-(3-chloro-2-fluoro-6-(1H-tetrazol-1-yl)phenyl)-4′-oxo-6′,7′-dihydro-4′H-spiro[cyclopropane-1,8′-pyrrolo[1,2-a]pyrimidine]-6′-carboxylate (150 mg, 0.22 mmol, 1.0 equiv) in toluene (5.0 mL) and CH₃COOH (0.5 mL) was added NH₄OAc (173 mg, 2.24 mmol, 10.0 equiv). The reaction mixture was stirred for 2 h at 100° C. The reaction mixture was concentrated to dryness under reduced pressure to yield a residue, which was purified by column chromatography on silica gel with MeOH/DCM (0-15%) & SFC to yield (S)-2′-(3-Chloro-2-fluoro-6-(1H-tetrazol-1-yl)phenyl)-6′-(4-(3-fluoro-2-((R)-3,3,3-trifluoro-2-hydroxypropoxy)pyridin-4-yl)-1H-imidazol-2-yl)-6′,7′-dihydro-4′H-spiro[cyclopropane-1,8′-pyrrolo[1,2-a]pyrimidin]-4′-one as a white solid.

LC/MS: mass calculated for C₂₇H₁₉ClF₅N₉O₃: 647.12, measured: 648.05 [M+H]⁺. ¹H NMR (300 MHz, DMSO-d₆) δ 12.71 (s, 1H), 9.73 (s, 1H), 8.05 (t, J=8.7 Hz, 1H), 7.93 (d, J=5.3 Hz, 1H), 7.74 (d, J=8.6 Hz, 1H), 7.64-7.67 (m, 1H), 7.60 (t, J=5.1 Hz, 1H), 6.68 (d, J=6.2 Hz, 1H), 6.54 (d, J=2.3 Hz, 1H), 5.75 (dd, J=9.4, 3.3 Hz, 1H), 4.52-4.58 (m, 1H), 4.42-4.50 (m, 2H), 2.72-2.82 (m, 1H), 2.19-2.27 (m, 1H), 1.00-1.15 (m, 2H), 0.77-0.88 (m, 2H). ¹⁹F NMR (282 MHz, DMSO-d₆) δ −76.15, −113.62, −143.16.

### Example 75: (R*)-2′-(3-Chloro-2-fluoro-6-(1H-tetrazol-1-yl)phenyl)-6′-(5-(3-fluoro-2-(1-hydroxycyclopropyl)pyridin-4-yl)-1H-imidazol-2-yl)-6′,7′-dihydro-4′H-spiro[cyclopropane-1,8′-pyrrolo[1,2-a]pyrimidin]-4′-one

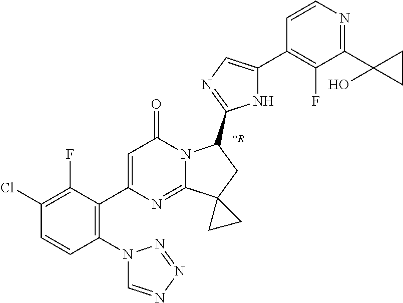

LC/MS: mass calculated for C₂₇H₂₀ClF₂N₉O₂: 575.14, measured: 576.05 [M+H]⁺. ¹H NMR (400 MHz, DMSO-d₆) δ 12.66 (s, 1H), 9.73 (s, 1H), 8.21-8.26 (m, 1H), 8.01-8.08 (m, 1H), 7.85 (t, J=5.2 Hz, 1H), 7.74 (dd, J=8.7, 1.5 Hz, 1H), 7.64-7.70 (m, 1H), 6.55 (d, J=2.2 Hz, 1H), 6.09 (s, 1H), 5.76 (dd, J=9.4, 3.4 Hz, 1H), 2.74-2.85 (m, 1H), 2.22 (dd, J=13.1, 3.4 Hz, 1H), 0.94-1.18 (m, 6H), 0.73-0.88 (m, 2H). ¹⁹F NMR (376 MHz, DMSO-d₆) δ −103.51, −125.54.

### Example 76 (S)-6′-(5-(6-amino-2-fluoropyridin-3-yl)-4-fluoro-1H-imidazol-2-yl)-2′-(3-chloro-2-fluoro-6-(1H-tetrazol-1-yl)phenyl)-6′,7′-dihydro-4′H-spiro[cyclopropane-1,8′-pyrrolo[1,2-a]pyrimidin]-4′-one

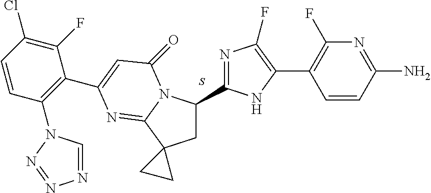

### Step 1: N-(5-(2-(2′-(3-Chloro-2-fluoro-6-(1H-tetrazol-1-yl)phenyl)-4′-oxo-6′,7′-dihydro-4′H-spiro[cyclopropane-1,8′-pyrrolo[1,2-a]pyrimidin]-6′-yl)-4-fluoro-1H-imidazol-5-yl)-6-fluoropyridin-2-yl)acetamide

To a solution of N-(5-(2-(2′-(3-chloro-2-fluoro-6-(1H-tetrazol-1-yl)phenyl)-4′-oxo-6′,7′-dihydro-4′H-spiro[cyclopropane-1,8′-pyrrolo[1,2-a]pyrimidin]-6′-yl)-1H-imidazol-5-yl)-6-fluoropyridin-2-yl)acetamide (280 mg, 0.48 mmol, 1.0 equiv) in DCM (10 mL) and acetone (10 mL) was added NaHCO₃ (204 mg, 2.43 mmol, 5.0 equiv), followed by the addition of N-fluoro-N-(phenylsulfonyl)benzenesulfonamide (459 mg, 1.47 mmol, 3.0 equiv). The reaction mixture was stirred at 50° C. for 5 h, then cooled to room temperature and purified by reverse column chromatography with CH₃CN/water (5%-50%) & SFC to yield N-(5-(2-(2′-(3-chloro-2-fluoro-6-(1H-tetrazol-1-yl)phenyl)-4′-oxo-6′,7′-dihydro-4′H-spiro[cyclopropane-1,8′-pyrrolo[1,2-a]pyrimidin]-6′-yl)-4-fluoro-1H-imidazol-5-yl)-6-fluoropyridin-2-yl)acetamide as a light yellow solid.

### Step 2: (R)-6′-(5-(6-Amino-2-fluoropyridin-3-yl)-4-fluoro-1H-imidazol-2-yl)-2′-(3-chloro-2-fluoro-6-(1H-tetrazol-1-yl)phenyl)-6′,7′-dihydro-4′H-spiro[cyclopropane-1,8′-pyrrolo[1,2-a]pyrimidin]-4′-one

To a solution of N-(5-(2-(2′-(3-chloro-2-fluoro-6-(1H-tetrazol-1-yl)phenyl)-4′-oxo-6′,7′-dihydro-4′H-spiro[cyclopropane-1,8′-pyrrolo[1,2-a]pyrimidin]-6′-yl)-4-fluoro-1H-imidazol-5-yl)-6-fluoropyridin-2-yl)acetamide (115 mg, 0.19 mmol, 1.0 equiv) in THE (5 mL) was added 2M HCl (5 mL). The mixture was stirred for 3 h at 50° C., then cooled to room temperature and concentrated under vacuum. The residue was purified by reverse column chromatography with CH₃CN/0.05% TFA water (5%-50%) to yield a residue, which was purified by Chiral-HPLC with MtBE(0.1% DEA):EtOH=92:8 to yield (R)-6′-(5-(6-amino-2-fluoropyridin-3-yl)-4-fluoro-1H-imidazol-2-yl)-2′-(3-chloro-2-fluoro-6-(1H-tetrazol-1-yl)phenyl)-6′,7′-dihydro-4′H-spiro[cyclopropane-1,8′-pyrrolo[1,2-a]pyrimidin]-4′-one as a white solid.

LC/MS mass calculated for: C₂₄H₁₆ClF₃N₁₀O: 552.91, measured (ES, m/z): 553.10 [M+H]⁺. ¹H NMR (400 MHz, Methanol-d₄): δ 9.41 (s, 1H), 7.87 (dd, J=8.7, 7.5 Hz, 1H), 7.68 (dd, J=10.0, 8.3 Hz, 1H), 7.57 (dd, J=8.6, 1.6 Hz, 1H), 6.54 (d, J=2.6 Hz, 1H), 6.46 (dd, J=8.3, 1.8 Hz, 1H), 5.71-5.73 (m, 1H), 2.78 (dd, J=13.2, 9.6 Hz, 1H), 2.35 (dd, J=13.2, 4.2 Hz, 1H), 1.00-1.17 (m, 3H), 0.85-0.96 (m, 1H). ¹⁹F NMR: (376 MHz, Methanol-d₄): δ−74.45, −76.94, −114.26, −138.15.

### Example 77: (6S*)-2-(3-Chloro-2-fluoro-6-(1H-tetrazol-1-yl)phenyl)-6-(5-(3-fluoro-2-(hydroxymethyl)pyridin-4-yl)-1H-imidazol-2-yl)-8-methyl-7,8-dihydropyrrolo[1,2-a]pyrimidin-4(6H)-one

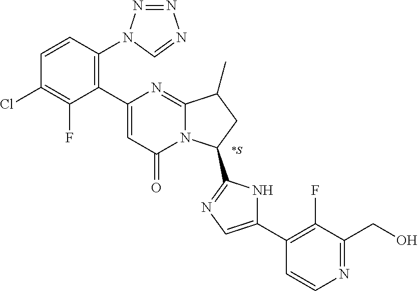 [SMILES: CC1C[C@@H](c2ncc(-c3ccnc(CO)c3F)[nH]2)n2c1nc(-c1c(-n3cnnn3)ccc(Cl)c1F)cc2=O]

LC/MS: mass calculated for C₂₄H₁₈ClF₂N₉O₂: 537.12, measured: 538.10 [M+H]⁺. ¹H NMR (400 MHz, DMSO-d₆) δ 9.60-9.79 (m, 1H), 8.36 (s, 1H), 7.93-8.12 (m, 2H), 7.78-7.92 (m, 1H), 7.71 (d, J=8.6 Hz, 1H), 6.50-6.67 (m, 1H), 5.50-5.77 (m, 1H), 4.64-4.83 (m, 2H), 3.30-3.48 (m, 1H), 1.80-2.44 (m, 3H), 0.78-1.03 (m, 3H). ¹⁹F NMR (376 MHz, DMSO-d₆) δ −74.22, −113.76, −128.90

### Example 78: (6R*)-2-(3-Chloro-2-fluoro-6-(1H-tetrazol-1-yl)phenyl)-6-(5-(3-fluoro-2-(hydroxymethyl)pyridin-4-yl)-1H-imidazol-2-yl)-8-methyl-7,8-dihydropyrrolo[1,2-a]pyrimidin-4(6H)-one

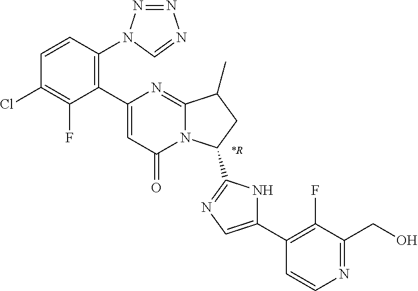 [SMILES: CC1C[C@H](c2ncc(-c3ccnc(CO)c3F)[nH]2)n2c1nc(-c1c(-n3cnnn3)ccc(Cl)c1F)cc2=O]

LC/MS: mass calculated for C₂₄H₁₈ClF₂N₉O₂: 537.12, measured: 538.10 [M+H]⁺. ¹H NMR (400 MHz, DMSO-d₆) δ 9.65-9.83 (m, 1H), 8.36 (s, 1H), 7.87-8.15 (m, 2H), 7.67-7.87 (m, 2H), 6.50-6.70 (m, 1H), 5.55-5.76 (m, 1H), 4.55-4.8. (m, 2H), 3.35-3.48 (m, 1H), 2.12-2.51 (m, 3H), 0.95-1.05 (m, 3H)¹⁹F NMR (376 MHz, DMSO-d₆) δ −73.99, −113.76, −129.32.

### Example 79 (6R)-6-(4-(5-aminopyrazin-2-yl)-1H-imidazol-2-yl)-2-(3-chloro-2-fluoro-6-(4-(trifluoromethyl)-1H-1,2,3-triazol-1-yl)phenyl)-8-methyl-7,8-dihydropyrrolo[1,2-a]pyrimidin-4(6H)-one

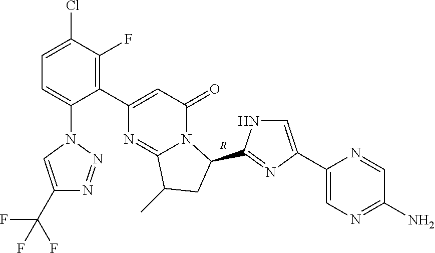

LC/MS mass calculated for: C₂₄H₁₇ClF₄N₁₀O: 572.91, measured (ES, m/z): 573.10 [M+H]⁺. ¹H NMR: (300 MHz, DMSO-d₆) δ 12.27-12.34 (m, 1H), 9.25-9.31 (m, 1H), 8.33 (s, 1H), 8.05 (t, J=8.2 Hz, 1H), 7.72-7.85 (m, 2H), 7.35 (s, 1H), 6.58 (s, 1H), 6.29 (s, 1H), 5.45-5.56 (m, 1H), 2.91-3.11 (m, 1H), 2.65-2.85 (m, 1H), 1.80-2.00 (m, 1H), 1.02-1.20 (m, 3H). ¹⁹F NMR: (282 MHz, DMSO-d₆) δ −59.63, −114.07.

### Example 80 (6S)-6-(4-(5-aminopyrazin-2-yl)-1H-imidazol-2-yl)-2-(3-chloro-2-fluoro-6-(4-(trifluoromethyl)-1H-1,2,3-triazol-1-yl)phenyl)-8-methyl-7,8-dihydropyrrolo[1,2-a]pyrimidin-4(6H)-one

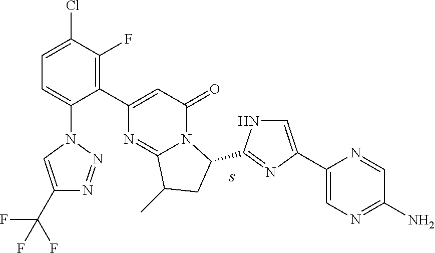

### Step 1: N-(5-bromopyrazin-2-yl)acetamide

A mixture of 2-Amino-5-bromopyrazine (2 g, 11.49 mmol, 1 eq) and acetic anhydride (10 mL) was at 45° C. for 5 h. The solid was collected by filtration, and dried to yield N-(5-bromopyrazin-2-yl)acetamide as a yellow solid. LC/MS mass calculated for: C₆H₆BrN₃O: 214.97, measured (ES, m/z): 218.00 [M+H+2]⁺.

### Step 2: N-(5-(1-ethoxyvinyl)pyrazin-2-yl)acetamide

To a solution of N-(5-bromopyrazin-2-yl)acetamide (1.3 g, 6.02 mmol, 1 eq) in 1,4-dioxane (20 mL) were added tributyl(1-ethoxyvinyl)tin (2.4 g, 6.62 mmol, 1.1 eq) and tetrakis(triphenylphosphine)palladium (695 mg, 0.60 mmol, 0.05 eq). The resulting mixture was maintained under nitrogen and stirred at 100° C. for 3 h, then cooled to room temperature and quenched with water. The reaction mixture was extracted with ethyl acetate. The organic layers were combined, washed with brine, dried and concentrated under vacuum. The residue was purified by silica gel chromatography (20-80% ethyl acetate/petroleum ether) to yield 1.2 g of N-(5-(1-ethoxyvinyl)pyrazin-2-yl)acetamide as a brown solid. LC/MS mass calculated for: C₁₀H₁₃N₃O₂:207.10, measured (ES, m/z): 208.25 [M+H]⁺.

### Step 3: N-(5-(2-bromoacetyl)pyrazin-2-yl)acetamide

To a solution of N-(5-(1-ethoxyvinyl)pyrazin-2-yl)acetamide (1.1 g, 5.07 mmol, 1 eq) in tetrahydrofuran (30 mL) and water (10 mL) was added N-bromosuccinimide (1.1 g, 6.08 mmol, 1.2 eq). The reaction mixture was stirred at room temperature for 2 h, then quenched with water. The reaction mixture was extracted with ethyl acetate. The organic layers were combined, washed with brine, dried and concentrated under vacuum. The residue was purified by silica gel chromatography (20-80% ethyl acetate/petroleum ether) to yield 0.7 g of N-(5-(2-bromoacetyl)pyrazin-2-yl)acetamide as a solid. LC/MS mass calculated for: C₈H₉BrN₃O₂: 256.98, measured (ES, m/z): 258.10 [M+H]⁺.

### Step 4: 2-(5-acetamidopyrazin-2-yl)-2-oxoethyl 2-(3-chloro-2-fluoro-6-(4-(trifluoromethyl)-1H-1,2,3-triazol-1-yl)phenyl)-8-methyl-4-oxo-4,6,7,8-tetrahydropyrrolo[1,2-a]pyrimidine-6-carboxylate

To a solution of (S)-2-(3-chloro-2-fluoro-6-(4-(trifluoromethyl)-1H-1,2,3-triazol-1-yl)phenyl)-8-methyl-4-oxo-4,6,7,8-tetrahydropyrrolo[1,2-a]pyrimidine-6-carboxylic acid (0.2 g, 0.44 mmol, 1 eq) in acetonitrile (10 mL) was added potassium carbonate (181 mg, 1.31 mmol, 3 eq). The reaction mixture was stirred at room temperature 0.5 h, then N-(5-(2-bromoacetyl)pyrazin-2-yl)acetamide (169 mg, 0.66 mmol, 1 eq) was added. The reaction mixture was stirred at room temperature for 2 h, concentrated under vacuum. The residue was purified by silica gel chromatography (0-10% DCM/MeOH) to yield 2-(5-acetamidopyrazin-2-yl)-2-oxoethyl 2-(3-chloro-2-fluoro-6-(4-(trifluoromethyl)-1H-1,2,3-triazol-1-yl)phenyl)-8-methyl-4-oxo-4,6,7,8-tetrahydropyrrolo[1,2-a]pyrimidine-6-carboxylate as a brown solid. LC/MS mass calculated for: C₂₆H₁₉ClF₄N₈O₅: 634.11, measured (ES, m/z): 635.10 [M+H]⁺

### Step 5: N-(5-(2-(2-(3-chloro-2-fluoro-6-(4-(trifluoromethyl)-1H-1,2,3-triazol-1-yl)phenyl)-8-methyl-4-oxo-4,6,7,8-tetrahydropyrrolo[1,2-a]pyrimidin-6-yl)-1H-imidazol-4-yl)pyrazin-2-yl)acetamide

To a solution of 2-(5-acetamidopyrazin-2-yl)-2-oxoethyl 2-(3-chloro-2-fluoro-6-(4-(trifluoromethyl)-1H-1,2,3-triazol-1-yl)phenyl)-8-methyl-4-oxo-4,6,7,8-tetrahydropyrrolo[1,2-a]pyrimidine-6-carboxylate (100 mg, 0.16 mmol, 1 eq) in toluene (15 ml) and acetic acid (0.8 ml) was added ammonium acetate (364 mg, 4.73 mmol, 30 eq). The reaction mixture was stirred at 100° C. 2 h, then cooled to room temperature. The resulting mixture was concentrated under vacuum. The residue was purified by silica gel chromatography (0-20% ethyl acetate/petroleum ether) to yield the N-(5-(2-(2-(3-chloro-2-fluoro-6-(4-(trifluoromethyl)-1H-1,2,3-triazol-1-yl)phenyl)-8-methyl-4-oxo-4,6,7,8-tetrahydropyrrolo[1,2-a]pyrimidin-6-yl)-1H-imidazol-4-yl)pyrazin-2-yl)acetamide. LC/MS mass calculated for: C₂₆H₁₉ClF₄N₁₀O₂: 614.13, measured (ES, m/z): 615.10 [M+H]⁺

### Step 6: (6S)-6-(4-(5-aminopyrazin-2-yl)-1H-imidazol-2-yl)-2-(3-chloro-2-fluoro-6-(4-(trifluoromethyl)-1H-1,2,3-triazol-1-yl)phenyl)-8-methyl-7,8-dihydropyrrolo[1,2-a]pyrimidin-4(6H)-one

To a solution N-(5-(2-(2-(3-chloro-2-fluoro-6-(4-(trifluoromethyl)-1H-1,2,3-triazol-1-yl)phenyl)-8-methyl-4-oxo-4,6,7,8-tetrahydropyrrolo[1,2-a]pyrimidin-6-yl)-1H-imidazol-4-yl)pyrazin-2-yl)acetamide (100 mg, 0.16 mmol, 1 eq) in tetrahydrofuran (10 mL) was added 2 M HCl (10 mL, 20.00 mmol, 122.988 eq). The reaction mixture was stirred 1 h at 50° C., then cooled to room temperature and concentrated under vacuum. The residue was purified by reverse column chromatography with CH₃CN/0.05% TFA water (5%-80%) to yield a residue, which was purified by Prep-HPLC with CH₃CN/0.05% TFA water to yield (6R)-6-(4-(5-aminopyrazin-2-yl)-1H-imidazol-2-yl)-2-(3-chloro-2-fluoro-6-(4-(trifluoromethyl)-1H-1,2,3-triazol-1-yl)phenyl)-8-methyl-7,8-dihydropyrrolo[1,2-a]pyrimidin-4(6H)-one as an white solid and (6S)-6-(4-(5-aminopyrazin-2-yl)-1H-imidazol-2-yl)-2-(3-chloro-2-fluoro-6-(4-(trifluoromethyl)-1H-1,2,3-triazol-1-yl)phenyl)-8-methyl-7,8-dihydropyrrolo[1,2-a]pyrimidin-4(6H)-one as an white solid.

LC/MS mass calculated for: C₂₄H₁₇ClF₄N₁₀O: 572.91, measured (ES, m/z): 573.10[M+H]⁺. ¹H NMR: (300 MHz, DMSO-d₆) δ 12.27-12.34 (m, 1H), 9.25-9.31 (m, 1H), 8.33 (s, 1H), 8.05 (t, J=8.2 Hz, 1H), 7.72-7.85 (m, 2H), 7.20-7.38 (m, 1H), 6.52-6.62 (m, 1H), 6.47 (s, 1H), 6.30 (s, 1H), 5.45-5.56 (m, 1H), 2.91-3.11 (m, 1H), 2.65-2.85 (m, 1H), 1.80-2.00 (m, 1H), 1.02-1.20 (m, 3H). ¹⁹F NMR: (282 MHz, DMSO-d₆) δ −59.63, −114.07.

### Example 81 (S)-6′-(5-(3-Aminobenzo[d]isothiazol-6-yl)-1H-imidazol-2-yl)-2′-(3-chloro-2-fluoro-6-(1H-tetrazol-1-yl)phenyl)-6′,7′-dihydro-4′H-spiro[cyclopropane-1,8′-pyrrolo[1,2-a]pyrimidin]-4′-one

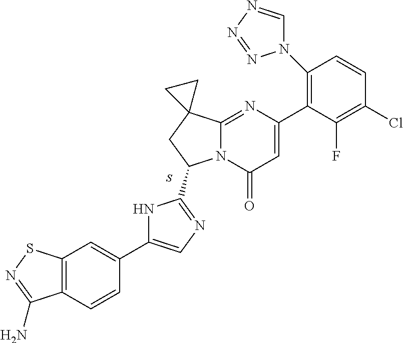

LC/MS mass calculated for: C₂₆H₁₈ClFN₁₀OS: 573.01, measured (ES, m/z): 573.10[M+H]⁺. H-NMR: (300 MHz, DMSO-d₆) δ 12.37-12.8 (m, 1H), 9.73 (d, J=5.2 Hz, 1H), 8.26 (s, 1H), 8.03 (d, J=8.2 Hz, 2H), 7.70-7.81 (m, 3H), 6.70 (s, 2H), 6.54 (s, 1H), 5.71-5.74 (m, 1H), 2.75-2.92 (m, 1H), 2.20-2.30 (m, 1H), 1.03-1.15 (m, 2H), 0.77-0.9 (m, 2H). 19F NMR: (282 MHz, DMSO-d₆) δ −113.49.

### Example 82 (R)-6′-(5-(3-Aminobenzo[d]isothiazol-6-yl)-1H-imidazol-2-yl)-2′-(3-chloro-2-fluoro-6-(1H-tetrazol-1-yl)phenyl)-6′,7′-dihydro-4′H-spiro[cyclopropane-1,8′-pyrrolo[1,2-a]pyrimidin]-4′-one

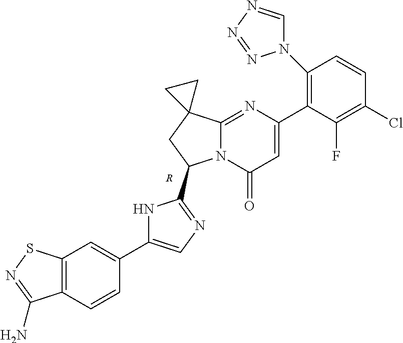

LC/MS mass calculated for: C₂₆H₁₈ClFN₁₀OS: 573.01, measured (ES, m/z): 573.10 [M+H]⁺. ¹H NMR: (300 MHz, DMSO-d₆) δ 12.37-12.89 (m, 1H), 9.73 (s, 1H), 8.26 (s, 1H), 7.98-8.10 (m, 2H), 7.74 (d, J=11.2 Hz, 3H), 6.70 (s, 1H), 6.54 (s, 1H), 5.70-5.74 (m, 1H), 2.75-2.89 (m, 1H), 2.22-2.30 (m, 1H), 1.03-1.15 (m, 2H), 0.77-0.9 (m, 2H). ¹⁹F NMR: (282 MHz, DMSO-d₆) δ −113.49.

### Example 83 (6*S,8*S)-6-(5-(5-Amino-3-(trifluoromethyl) pyrazin-2-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

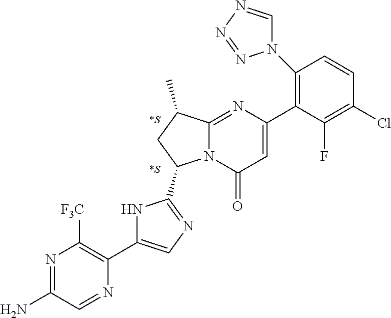

LC/MS mass calculated for: C₂₃H₁₆ClF₄N₁₁O: 573.90, measured (ES, m/z): 574.00 [M+H]⁺. ¹H NMR: (400 MHz, Methanol-d₄): δ 9.47 (s, 1H), 8.12 (s, 1H), 7.89 (dd, J=8.7, 7.5 Hz, 1H), 7.60 (dd, J=8.7, 1.6 Hz, 1H), 7.20 (s, 1H), 6.63 (d, J=2.4 Hz, 1H), 5.76 (d, J=9.0 Hz, 1H), 3.49-3.62 (m, 1H), 2.53-2.68 (m, 1H), 2.11-2.33 (m, 1H), 1.07 (d, J=6.9 Hz, 3H). ¹⁹F NMR: (376 MHz, Methanol-d₄): δ −65.22, −114.50.

### Example 84 (6*S,8*S)-6-(5-(5-Amino-3-methoxypyrazin-2-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

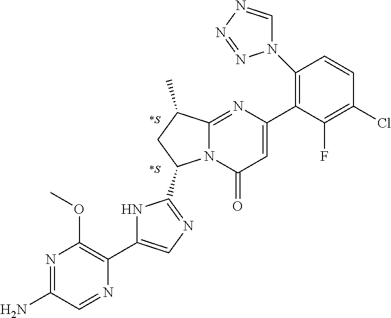

### Step 1: 5-bromo-6-methoxypyrazin-2-amine

To a solution of 6-methoxypyrazin-2-amine (1.0 g, 7.99 mmol, 1 equiv) in DMF (20 mL) was added NBS (1.4 g, 7.99 mmol, 1.0 equiv) stepwise at 0° C. The mixture was stirred for 2 h at room temperature, then quenched with water, extracted with EA, washed with brine, dried over Na₂SO₄ and concentrated under vacuum. The residue was purified by silica gel chromatography with EA/PE (0-40%) to yield 5-bromo-6-methoxypyrazin-2-amine as a light yellow solid. LC/MS: mass calculated for C₅H₆BrN₃O: 202.97, measured: 203.90 [M+H]⁺, 205.90 [M+2+H]⁺.

### Step 2: 1-(5-amino-3-methoxypyrazin-2-yl)ethan-1-one

To a solution of 5-bromo-6-methoxypyrazin-2-amine (1.4 g, 5.78 mmol, 1 equiv) in 1,4-dioxane (20 mL) was added 1-ethoxyvinyl-tri-n-butyltin (2.3 g, 6.364 mmol, 1.1 equiv) and Pd(PPh₃)₄ (334 mg, 0.29 mmol, 0.05 equiv) under N₂. The reaction mixture was stirred overnight at 100° C., then cooled to room temperature and quenched with water, extracted with EA, dried over Na₂SO₄ and concentrated under vacuum. The residue was purified by silica gel chromatography with EA/PE (0-70%) to yield 1-(5-amino-3-methoxypyrazin-2-yl)ethan-1-one as a light yellow solid. LC/MS: mass calculated for C₇H₉N₃O₂: 167.07, measured: 168.10 [M+H]⁺.

### Step 3: 1-(5-amino-3-methoxypyrazin-2-yl)-2-bromoethan-1-one

To a solution of 1-(5-amino-3-methoxypyrazin-2-yl)ethan-1-one (0.5 g, 2.81 mmol, 1 equiv) in acetic acid (10 mL) was added hydrogen bromide solution in acetic acid (1.4 g, 5.62 mmol, 2 equiv) followed by the addition of pyridinium tribromide (854 mg, 2.67 mmol, 0.95 equiv) slowly. The reaction mixture was stirred for 2 h at room temperature. The solids were collected by filtration and washed with diethyl ether, then evaporated under vacuum. This resulted 1-(5-amino-3-methoxypyrazin-2-yl)-2-bromoethan-1-one hydrobromide as a grey solid. LC/MS: mass calculated for C₇H₉BrN₃O₂: 244.98, measured: 246.00 [M+H]⁺, 248.00 [M+2+H]⁺.

### Step 4: 2-(5-amino-3-methoxypyrazin-2-yl)-2-oxoethyl (8S)-2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate

To a solution of (6S,8S)-2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylic acid (120 mg, 0.31 mmol, 1.0 equiv) in DMF (5 mL) was added potassium carbonate (170 mg, 1.23 mmol, 4 equiv). After the mixture was stirred for 20 min, 1-(5-amino-3-methoxypyrazin-2-yl)-2-bromoethan-1-one hydrobromide (151 mg, 0.46 mmol, 1.5 equiv) was added. The reaction mixture was stirred for 2 h at room temperature, then concentrated and the resulting residue was purified by reverse column chromatography with CH₃CN/0.05% TFA water (5%-50%) to yield 2-(5-amino-3-methoxypyrazin-2-yl)-2-oxoethyl (8S)-2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate as light brown oil. LC/MS: mass calculated for C₂₃H₁₉ClFN₉O₅: 555.12, measured: 578.00 [M+Na]⁺.

### Step 5: (6*S,8*S)-6-(5-(5-amino-3-methoxypyrazin-2-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

To a solution of 2-(5-amino-3-methoxypyrazin-2-yl)-2-oxoethyl (8S)-2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate (150 mg, 0.27 mmol, 1.0 equiv) in toluene (10 mL) was added ammonium acetate (624 mg, 8.10 mmol, 30 equiv) followed by the addition of acetic acid (0.5 mL) under N₂. The reaction mixture was stirred for 2 h at 100° C., then cooled to room temperature and concentrated under vacuum. The residue was purified by silica gel chromatography with MeOH/DCM (0-10%) to yield a second residue, which was purified by Prep-HPLC with CH₃CN/0.05% TFA water to yield (6*S,8*S)-6-(5-(5-amino-3-methoxypyrazin-2-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one as a light yellow solid.

LC/MS mass calculated for: C₂₃H₁₉ClFN₁₁O₂: 535.93, measured (ES, m/z): [M+H]⁺. ¹H NMR: (400 MHz, Methanol-d₄): δ 9.49 (s, 1H), 7.88-7.97 (m, 1H), 7.54-7.66 (m, 3H), 6.70 (d, J=2.3 Hz, 1H), 5.93 (dd, J=9.5, 3.6 Hz, 1H), 3.37-3.52 (m, 1H), 2.56-2.66 (m, 1H), 2.32-2.46 (m, 1H), 1.10 (d, J=7.1 Hz, 3H). ¹⁹F NMR (376 MHz, Methanol-d₄): δ −77.01, −114.56.

### Example 85 (6*R,8*S)-6-(5-(5-Amino-3-(difluoromethyl) pyrazin-2-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

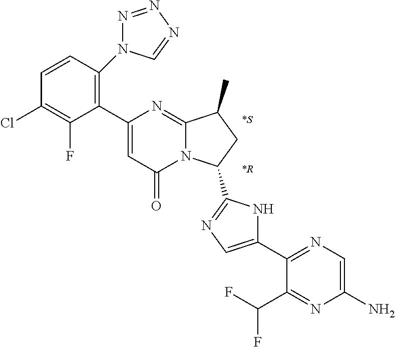

### Step 1: Methyl 6-amino-3-bromopyrazine-2-carboxylate

To a solution of methyl 6-aminopyrazine-2-carboxylate (2.3 g, 15.02 mmol, 1 equiv) in DMF (30 mL) was added NBS (2.7 g, 15.02 mmol, 1.0 equiv) dropwise at 0° C. The mixture was stirred for 2 h at room temperature, then quenched with water, extracted with EA, washed with brine, dried over Na₂SO₄ and concentrated under vacuum. The residue was purified by silica gel chromatography with EA/PE (0-60%) to yield methyl 6-amino-3-bromopyrazine-2-carboxylate as a light yellow solid. LC/MS: mass calculated for C₆H₆BrN₃O₂: 230.96, measured: 234.00 [M+2+H]⁺.

### Step 2: Methyl 6-acetamido-3-bromopyrazine-2-carboxylate

Methyl 6-amino-3-bromopyrazine-2-carboxylate (2.3 g, 9.91 mmol, 1 equiv) was dissolved in acetic anhydride (20 mL). The mixture was stirred overnight at room temperature. The solid was collected by filtration and washed with EA/PE (1/10), then evaporated under vacuum to yield methyl 6-acetamido-3-bromopyrazine-2-carboxylate as a light yellow solid. LC/MS: mass calculated for C₈H₉BrN₃O₃: 272.97, measured: 276.05 [M+2+H]⁺.

### Step 3: N-(5-Bromo-6-(hydroxymethyl)pyrazin-2-yl)acetamide

To a solution of methyl 6-acetamido-3-bromopyrazine-2-carboxylate (1.8 g, 6.57 mmol, 1 equiv) in water (40 mL) and THE (20 mL) was added sodium borohydride (1.2 g, 32.84 mmol, 5 equiv) in several batches. The mixture was stirred for 3 h at room temperature, then quenched with 1 M HCl and extracted with EA, dried over Na₂SO₄ and concentrated under vacuum to yield N-(5-bromo-6-(hydroxymethyl)pyrazin-2-yl)acetamide as a light yellow solid. LC/MS: mass calculated for C₇H₉BrN₃O₂: 244.98, measured: 248.10 [M+2+H]⁺.

### Step 4: N-(5-Bromo-6-formylpyrazin-2-yl)acetamide

To a solution of N-(5-bromo-6-(hydroxymethyl)pyrazin-2-yl)acetamide (790 mg, 3.21 mmol, 1 equiv) in DCM (30 mL) was added Dess-Martin periodinane (1.6 g, 3.85 mmol, 1.2 equiv). The mixture was stirred for 2 h at room temperature, then filtered and washed with DCM. The filtrate was concentrated under vacuum. The residue was purified by silica gel chromatography with EA/PE (0-60%) to yield N-(5-bromo-6-formylpyrazin-2-yl)acetamide as light yellow oil. LC/MS: mass calculated for C₇H₉BrN₃O₂: 242.96, measured: 246.10 [M+2+H]⁺.

### Step 5: N-(5-Bromo-6-(difluoromethyl)pyrazin-2-yl)acetamide

To a solution of N-(5-bromo-6-formylpyrazin-2-yl)acetamide (0.4 g, 1.64 mmol, 1 equiv) in DCM (10 mL) was added DAST (528 mg, 3.28 mmol, 2 equiv). The mixture was stirred for 2 h at room temperature, then quenched with NaHCO₃ (aq.), extracted with DCM, dried over Na₂SO₄ and concentrated under vacuum. The residue was purified by silica gel chromatography with EA/PE (0-30%) to yield N-(5-bromo-6-(difluoromethyl)pyrazin-2-yl)acetamide as light yellow oil. LC/MS: mass calculated for C₇H₆BrF₂N₃O: 264.97, measured: 268.00 [M+2+H]⁺.

### Step 6: N-(6-(Difluoromethyl)-5-(1-ethoxyvinyl)pyrazin-2-yl)acetamide

To a solution of N-(5-bromo-6-cyclopropylpyrazin-2-yl)acetamide (240 mg, 0.937 mmol, 1 equiv) in 1,4-dioxane (10 mL) was added 1-ethoxyvinyl-tri-n-butyltin (372 mg, 1.03 mmol, 1.1 equiv) and Pd(PPh₃)₄ (108 mg, 0.09 mmol, 0.1 equiv) under N₂. The reaction mixture was stirred overnight at 100° C., then cooled to room temperature and quenched with water, extracted with EA, dried over Na₂SO₄ and concentrated under vacuum. The residue was purified by silica gel chromatography with EA/PE (0-50%) to yield N-(6-(difluoromethyl)-5-(1-ethoxyvinyl)pyrazin-2-yl)acetamide as a light yellow solid. LC/MS: mass calculated for C₁₁H₁₃F₂N₃O₂: 257.10, measured: 258.10 [M+2+H]⁺.

### Step 7: N-(5-(2-Bromoacetyl)-6-(difluoromethyl)pyrazin-2-yl)acetamide

To a solution of N-(6-cyclopropyl-5-(1-ethoxyvinyl)pyrazin-2-yl)acetamide (190 mg, 0.74 mmol, 1 equiv) in THE (6 mL) and water (2 mL) was added NBS (197 mg, 1.11 mmol, 1.5 equiv). The mixture was stirred for 2 h at room temperature, then extracted with EA, washed with brine, dried over Na₂SO₄ and concentrated under vacuum. The residue was purified by silica gel chromatography with EA/PE (0-30%) to yield N-(5-(2-bromoacetyl)-6-(difluoromethyl)pyrazin-2-yl)acetamide as a light yellow solid. LC/MS: mass calculated for C₈H₉BrF₂N₃O₂: 306.98, measured: 310.00 [M+2+H]⁺.

### Step 8: 2-(5-acetamido-3-(difluoromethyl)pyrazin-2-yl)-2-oxoethyl (8S)-2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate

To a solution of (6S,8S)-2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylic acid (0.17 g, 0.435 mmol, 1.0 equiv) in acetonitrile (10 mL) was added potassium carbonate (0.120 g, 0.870 mmol, 2 equiv). After the mixture was stirred for 20 min, N-(5-(2-bromoacetyl)-6-(difluoromethyl)pyrazin-2-yl)acetamide (0.174 g, 0.566 mmol, 1.3 equiv) was added, then concentrated under vacuum. The residue was purified by silica gel chromatography with MeOH/DCM (0-10%) to yield 2-(5-acetamido-3-(difluoromethyl)pyrazin-2-yl)-2-oxoethyl (6S,8S)-2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate as a light yellow solid.

LC/MS: mass calculated for C₂₅H₁₉ClF₃N₉O₅: 617.11, measured: 618.10 [M+H]⁺.

### Step 9: N-(5-(2-((8S)-2-(3-chloro-2-fluoro-6-(1H-tetrazol-1-yl)phenyl)-8-methyl-4-oxo-4,6,7,8-tetrahydropyrrolo[1,2-a]pyrimidin-6-yl)-1H-imidazol-5-yl)-6-(difluoromethyl)pyrazin-2-yl)acetamide

To a solution of 2-(5-acetamido-3-(difluoromethyl)pyrazin-2-yl)-2-oxoethyl (6S,8S)-2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate (120 mg, 0.20 mmol, 1.0 equiv) in toluene (10 mL) was added ammonium acetate (456 mg, 5.92 mmol, 30 equiv) followed by the addition of acetic acid (0.5 mL) under N₂.

The reaction mixture was stirred for 5 h at 100° C., then cooled to room temperature and concentrated under vacuum. The residue was purified by silica gel chromatography with MeOH/DCM (0-10%) to yield N-(5-(2-((8S)-2-(3-chloro-2-fluoro-6-(1H-tetrazol-1-yl)phenyl)-8-methyl-4-oxo-4,6,7,8-tetrahydropyrrolo[1,2-a]pyrimidin-6-yl)-1H-imidazol-5-yl)-6-(difluoromethyl)pyrazin-2-yl)acetamide as light yellow oil. LC/MS: mass calculated for C₂₅H₁₉ClF₃N₁₁O₂: 597.14, measured: 598.10 [M+H]⁺.

### Step 10: (6*R,8*S)-6-(5-(5-amino-3-(difluoromethyl) pyrazin-2-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

To a solution of N-(5-(2-((8S)-2-(3-chloro-2-fluoro-6-(1H-tetrazol-1-yl)phenyl)-8-methyl-4-oxo-4,6,7,8-tetrahydropyrrolo[1,2-a]pyrimidin-6-yl)-1H-imidazol-5-yl)-6-(difluoromethyl)pyrazin-2-yl)acetamide (80 mg, 0.13 mmol, 1 equiv) in THE (5 mL) was added 2 M HCl (5 mL). The reaction mixture was stirred for 2 h at 50° C., then cooled to room temperature and concentrated under vacuum. The residue was purified by reverse column chromatography with CH₃CN/water (5%-50%) to yield a residue, which was purified by MtBE (0.1% DEA):EtOH=90:10 to yield (6*S,8*S)-6-(5-(5-amino-3-(difluoromethyl)pyrazin-2-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one as a light yellow solid and (6*R,8*S)-6-(5-(5-amino-3-(difluoromethyl)pyrazin-2-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one 1-oxide as a light yellow solid.

LC/MS mass calculated for: C₂₃H₁₇ClF₃N₁₁O: 555.91, measured (ES, m/z): 556.15 [M+H]⁺. ¹H NMR: (400 MHz, Methanol-d₄): δ 9.43 (s, 1H), 8.06 (s, 1H), 7.89 (dd, J=8.7, 7.5 Hz, 1H), 7.24-7.64 (m, 3H), 6.58 (d, J=2.3 Hz, 1H), 5.58-5.68 (m, 1H), 3.09-3.20 (m, 1H), 2.82-2.91 (m, 1H), 2.04-2.23 (m, 1H), 1.24 (d, J=7.1 Hz, 3H). ¹⁹F NMR: (376 MHz, Methanol-d₄): δ −76.94, −114.48, −119.73.

### Example 86 (6*S,8*S)-6-(5-(5-Amino-3-(difluoromethyl)pyrazin-2-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

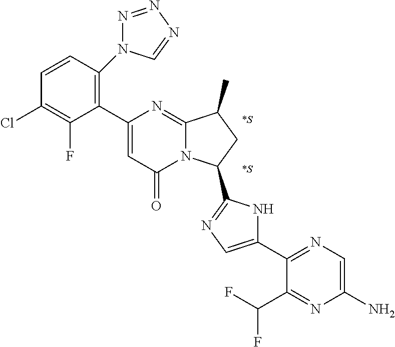

LC/MS mass calculated for: C₂₃H₁₇ClF₃N₁₁O: 555.91, measured (ES, m/z): 556.15 [M+H]⁺. ¹H NMR: (400 MHz, Methanol-d₄): δ 9.47 (s, 1H), 8.06 (s, 1H), 7.90 (dd, J=8.7, 7.5 Hz, 1H), 7.60 (dd, J=8.6, 1.6 Hz, 1H), 7.41 (s, 1H), 7.19 (t, J=54.2 Hz, 1H), 6.65 (d, J=2.4 Hz, 1H), 5.79 (dd, J=9.2, 1.8 Hz, 1H), 3.47-3.59 (m, 1H), 2.61-2.71 (m, 1H), 2.21-2.34 (m, 1H), 1.09 (d, J=7.0 Hz, 3H). ¹⁹F NMR: (376 MHz, Methanol-d₄): δ −77.00, −114.52, −119.39.

### Example 87: (6R*,8S*)-6-(5-(2-amino-3-fluoropyridin-4-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

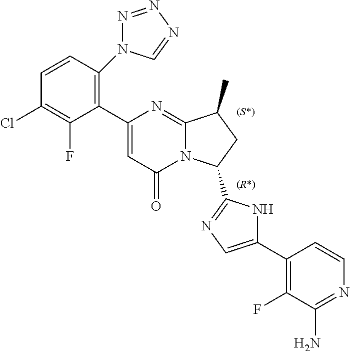 [SMILES: C[C@H]1C[C@H](c2ncc(-c3ccnc(N)c3F)[nH]2)n2c1nc(-c1c(-n3cnnn3)ccc(Cl)c1F)cc2=O]

### Step 1: 2-(2-Amino-3-fluoropyridin-4-yl)-2-oxoethyl-2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate

To a solution of 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylic acid (80 mg, 0.2 mmol, 0.8 equiv.) in DMF (1 mL) was added K₂CO₃ (53 mg, 0.3 mmol, 1.5 equiv.). The resulting mixture was stirred at room temperature for 0.5 h. 1-(2-amino-3-fluoropyridin-4-yl)-2-bromoethan-1-one (60 mg, 0.2 mmol, 1.0 equiv.) was added in portions. The reaction was stirred at 25° C. for 1 h. The resulting mixture was filtered. The residue obtained was purified by reverse-phase flash with CH₃CN/H₂O (0.05% TFA) (40-45%) to yield 2-(2-amino-3-fluoropyridin-4-yl)-2-oxoethyl-2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate as a yellow solid.

### Step 2: (6R*,8S*)-6-(5-(2-Amino-3-fluoropyridin-4-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

To a solution of 2-(2-amino-3-fluoropyridin-4-yl)-2-oxoethyl 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate (100 mg, 0.1 mmol, 1.0 equiv.) in acetic acid (0.4 mL) and toluene (2 mL) was added ammonium acetate (709 mg, 9.2 mmol, 50.0 equiv.). The resulting mixture was stirred at 100° C. for 1 h, then cooled to r.t and concentrated under vacuum. The residue was purified by reverse phase chromatography with ACN/water (0.05% TFA) (30-40%) to yield (6R*,8S*)-6-(5-(2-amino-3-fluoropyridin-4-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one as a white solid.

LC/MS: mass calculated for C₂₃H₁₇ClF₂N₁₀O: 522.12, measured: 523.20 [M+H]⁺. ¹H NMR (300 MHz, DMSO-d₆) δ 12.72 (s, 1H), 9.74 (s, 1H), 8.04-8.08 (m, 1H), 7.72-7.77 (m, 2H), 7.61 (s, 1H), 7.14 (s, 1H), 6.25-6.55 (m, 3H), 5.54-5.58 (m, 1H), 3.05-3.15 (m, 1H), 2.74-2.81 (m, 1H), 1.86-1.93 (m, 1H), 1.13-1.18 (m, 3H). ¹⁹F NMR (376 MHz, DMSO-d₆) δ −73.51, −113.79.

### Example 88: (6*S,8*S)-6-(5-(2-Amino-3-fluoropyridin-4-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

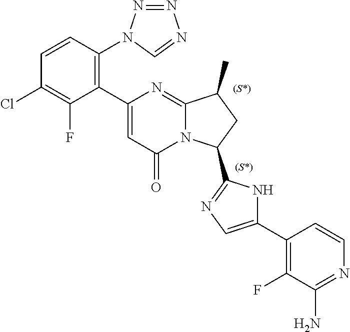 [SMILES: C[C@H]1C[C@@H](c2ncc(-c3ccnc(N)c3F)[nH]2)n2c1nc(-c1c(-n3cnnn3)ccc(Cl)c1F)cc2=O]

LC/MS: mass calculated for C₂₃H₁₇ClF₂N₁₀O: 522.12, measured: 523.20 [M+H]⁺. ¹H NMR (300 MHz, DMSO-d₆) δ 12.76 (s, 1H), 9.77 (s, 1H), 8.04-8.09 (m, 1H), 7.76-7.79 (m, 1H), 7.70-7.72 (m, 1H), 7.64-7.66 (m, 1H), 7.13 (s, 1H), 6.85 (brs, 2H), 6.64 (s, 1H), 5.66-5.68 (m, 1H), 2.39-2.44 (m, 1H), 2.33-2.34 (m, 1H), 2.16-2.24 (m, 1H), 0.98-1.00 (m, 3H). ¹⁹F NMR (376 MHz, DMSO-d₆) δ −73.51, −113.79.

### Example 89 (6*S,8S)-6-(5-(5-Amino-3-fluoropyrazin-2-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

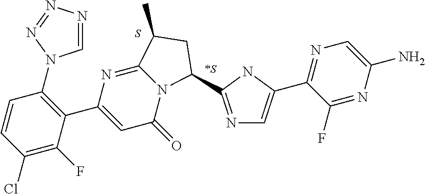

LC/MS mass calculated for: C₂₂H₁₆ClF₂N₁₁O: 523.12, measured (ES, m/z): 524.10 [M+H]⁺. ¹H NMR: (DMSO, 400 MHz) δ 12.59-12.61 (m, 1H), 9.77 (d, J=6.6 Hz, 1H), 8.02-8.10 (m, 1H), 7.73-7.87 (m, 2H), 7.03-7.33 (m, 1H), 7.00 (s, 1H), 6.56-6.80 (m, 2H), 5.30-5.73 (m, 1H), 3.50-3.52 (m, 1H), 2.06-2.19 (m, 1H), 1.97-2.05 (m, 1H), 0.94 (d, J=6.9 Hz, 3H). ¹⁹F NMR: (DMSO, 376 MHz) δ −78.89, −113.50, −113.72.

### Example 90 (6*R,8*S)-6-(5-(5-Amino-3-cyclopropylpyrazin-2-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

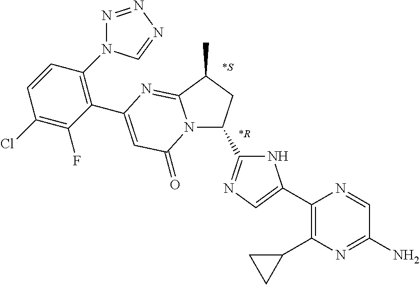 [SMILES: C[C@H]1C[C@H](c2ncc(-c3ncc(N)nc3C3CC3)[nH]2)n2c1nc(-c1c(-n3cnnn3)ccc(Cl)c1F)cc2=O]

LC/MS mass calculated for: C₂₅H₂₁ClFN₁₁O: 545.97, measured (ES, m/z): 546.15 [M+H]⁺. ¹H NMR: (400 MHz, Methanol-d₄): δ 9.44 (s, 1H), 7.89 (dd, J=8.7, 7.6 Hz, 1H), 7.68 (s, 1H), 7.60 (dd, J=8.6, 1.6 Hz, 1H), 7.25 (s, 1H), 6.53-6.59 (m, 1H), 5.60 (t, J=7.9 Hz, 1H), 3.24-3.30 (m, 1H), 3.07-3.18 (m, 1H), 2.76-2.91 (m, 1H), 2.04-2.18 (m, 1H), 1.23 (d, J=7.1 Hz, 3H), 0.98-1.09 (m, 2H), 0.80-0.98 (m, 2H). ¹⁹F NMR: (376 MHz, Methanol-d₄): δ −76.94, −114.44.

### Example 91 (6*S,8*S)-6-(5-(5-Amino-3-cyclopropylpyrazin-2-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

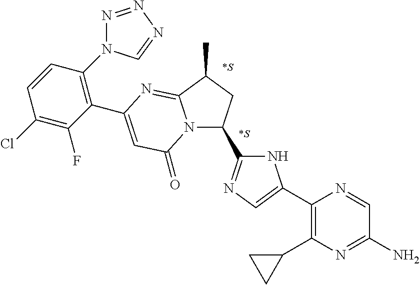 [SMILES: C[C@H]1C[C@@H](c2ncc(-c3ncc(N)nc3C3CC3)[nH]2)n2c1nc(-c1c(-n3cnnn3)ccc(Cl)c1F)cc2=O]

LC/MS mass calculated for: C₂₅H₂₁ClFN₁₁O: 545.97, measured (ES, m/z): 546.15 [M+H]⁺. ¹H NMR: (400 MHz, Methanol-d₄): δ 9.47 (s, 1H), 7.89 (dd, J=8.7, 7.5 Hz, 1H), 7.68 (s, 1H), 7.60 (dd, J=8.7, 1.6 Hz, 1H), 7.22 (s, 1H), 6.63 (s, 1H), 5.77 (d, J=9.2 Hz, 1H), 3.49-3.63 (m, 1H), 3.23-3.30 (m, 1H), 2.56-2.70 (m, 1H), 2.14-2.30 (m, 1H), 1.08 (d, J=7.0 Hz, 3H), 0.97-1.05 (m, 2H), 0.79-0.97 (m, 2H). ¹⁹F NMR: (376 MHz, Methanol-d₄): δ −76.94, −114.53.

### Example 92: 6-(2-((1S*,3S*)-7-(3-Chloro-2-fluoro-6-(1H-tetrazol-1-yl)phenyl)-1-methyl-5-oxo-1,2,3,5-tetrahydroindolizin-3-yl)-1H-imidazol-5-yl)-3,4-dihydro-1,7-naphthyridin-2(1H)-one

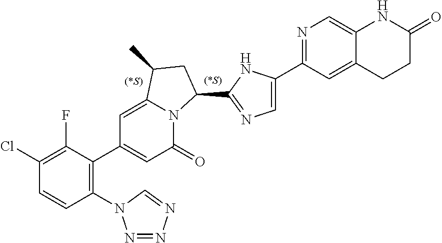 [SMILES: C[C@H]1C[C@@H](c2ncc(-c3cc4c(cn3)NC(=O)CC4)[nH]2)n2c1cc(-c1c(-n3cnnn3)ccc(Cl)c1F)cc2=O]

### Step 1: 6-Chloro-3,4-dihydro-1,7-naphthyridin-2(1H)-one

To a solution of ethyl (E)-3-(5-amino-2-chloropyridin-4-yl)acrylate (1 g, 4.41 mmol, 1.0 equiv) in AcOH (10 mL) was added Zn (0.3 g, 4.41 mmol, 1.0 equiv). The mixture stirred at room temperature for 2 h. The solid was filtered off and the filtrate was concentrated under vacuum. The residue was purified by silica gel chromatography (0-20% ethyl acetate/petroleum ether) to yield the 6-chloro-3,4-dihydro-1,7-naphthyridin-2(1H)-one as a white solid. LC/MS: mass calculated for C₈H₇ClN₂O: 182.02, measured: 183.10 [M+H]⁺.

### Step 2: 6-Acetyl-3,4-dihydro-1,7-naphthyridin-2(1H)-one

To a solution of 6-chloro-3,4-dihydro-1,7-naphthyridin-2(1H)-one (300 mg, 1.64 mmol, 1.0 equiv) in 1,4-dioxane (10 mL) was added tributyl(1-ethoxyvinyl)stannane (1.9 g, 5.26 mmol, 3.2 equiv), Pd(PPh₃)₂Cl₂ (83 mg, 0.16 mmol, 0.1 equiv). The resulting mixture was maintained under nitrogen and stirred at 100° C. overnight. After cooling to room temperature, the reaction was quenched with water (20 mL). The resulting mixture was extracted with ethyl acetate (3×20 mL). The organic layers were combined, dried over anhydrous sodium sulfate, filtered and concentrated to yield a residue. The residue was re-dissolved in THE (10 ml) and 2N HCl (1 mL) and the mixture was stirred for 2 h and extracted with ethyl acetate (3×20 mL). The organic layers were combined, dried over anhydrous sodium sulfate, filtered and concentrated. The residue obtained was purified by silica gel chromatography (0-80% ethyl acetate/petroleum ether) to yield the 6-acetyl-3,4-dihydro-1,7-naphthyridin-2(1H)-one as a yellow solid. LC/MS: mass calculated for C₁₀H₁₀N₂O₂: 190.07, measured: 191.10 [M+H]⁺.

### Step 3: 6-(2-Bromoacetyl)-3,4-dihydro-1,7-naphthyridin-2(1H)-one

To a solution of 6-acetyl-3,4-dihydro-1,7-naphthyridin-2(1H)-one (300 mg, 1.58 mmol, 1.0 equiv) and 48% HBr (0.1 mL) in AcOH (10 mL) was added Py-Br₃ (454 mg, 1.42 mmol, 0.9 equiv). The mixture was stirred at room temperature for 2 h. The residue was purified by C18 column with CH₃CN/0.05% TFA water (5%-45%) to yield the 6-(2-bromoacetyl)-3,4-dihydro-1,7-naphthyridin-2(1H)-one as a yellow solid. LC/MS: mass calculated for C₁₀H₉BrN₂O₂: 267.98, measured: 269.00 [M+H]⁺.

### Step 4: 2-Oxo-2-(2-oxo-1,2,3,4-tetrahydro-1,7-naphthyridin-6-yl)ethyl 7-(3-chloro-2-fluoro-6-(1H-tetrazol-1-yl)phenyl)-1-methyl-5-oxo-1,2,3,5-tetrahydroindolizine-3-carboxylate

To a solution of (6S,8S)-2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylic acid (200 mg, 0.51 mmol, 1.0 equiv) in DMF (1 mL) was added K₂CO₃ (106 mg, 0.77 mmol, 1.5 equiv). After 0.5 h of stirring, 6-(2-bromoacetyl)-3,4-dihydro-1,7-naphthyridin-2(1H)-one (165 mg 0.61 mmol, 1.2 equiv) was added. The mixture was stirred at room temperature for 1 h, then concentrated under vacuum. The residue was purified by reverse phase chromatography on C18 column with CH₃CN/0.05% TFA water (5%-40%) & SFC to yield the 2-oxo-2-(2-oxo-1,2,3,4-tetrahydro-1,7-naphthyridin-6-yl)ethyl (6S,8S)-2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate as a yellow oil.

### Step 5: 6-(2-((1 S*,3S*)-7-(3-Chloro-2-fluoro-6-(1H-tetrazol-1-yl)phenyl)-1-methyl-5-oxo-1,2,3,5-tetrahydroindolizin-3-yl)-1H-imidazol-5-yl)-3,4-dihydro-1,7-naphthyridin-2(1H)-one

To a solution of 2-oxo-2-(2-oxo-1,2,3,4-tetrahydro-1,7-naphthyridin-6-yl)ethyl (6S,8S)-2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate (330 mg, 0.57 mmol, 1.0 equiv) in toluene (10 mL) and acetic acid (0.3 mL) was added ammonium acetate (879 mg, 11.40 mmol, 20.0 equiv). The mixture was stirred at 100° C. for 1h, then concentrated under vacuum. The residue was purified by silica gel chromatography (0-20% MeOH/DCM) to yield a residue, which was purified by chiral-HPLC with (MtBE(0.1% DEA):EtOH=75:25) to yield 6-(2-((1S*,3S*)-7-(3-chloro-2-fluoro-6-(1H-tetrazol-1-yl)phenyl)-1-methyl-5-oxo-1,2,3,5-tetrahydroindolizin-3-yl)-1H-imidazol-5-yl)-3,4-dihydro-1,7-naphthyridin-2(1H)-one as an off-white solid.

LC/MS: mass calculated for C₂₇H₂₁ClFN₉O₂: 557.15, measured: 559.10 [M+H]⁺. ¹H NMR (400 MHz, DMSO-d₆) δ 12.23-12.80 (m, 1H), 10.18-10.45 (m, 1H), 9.78 (s, 1H), 7.95-8.19 (m, 2H), 7.75-7.85 (m, 1H), 7.64 (s, 1H), 7.29-7.55 (m, 1H), 6.62 (s, 1H), 5.50-5.79 (m, 1H), 3.38-3.58 (m, 2H), 2.90-3.04 (m, 2H), 2.40-2.42 (m, 1H), 2.25-2.40 (m, 1H), 2.03-2.22 (m, 1H), 0.89-1.03 (m, 3H). ¹⁹F NMR (376 MHz, DMSO-d₆) δ −73.40, −113.79.

### Example 93 (6S,8S)-6-(4-(5-Amino-3-chloropyrazin-2-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

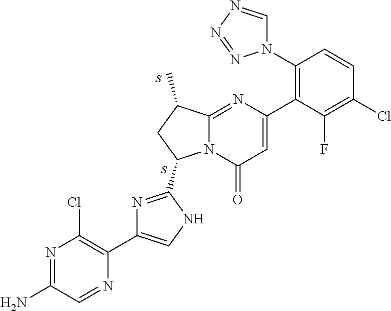

### Step 1: N-(5-Bromo-6-chloropyrazin-2-yl)acetamide

A mixture of 5-bromo-6-chloropyrazin-2-amine (2.5 g, 11.99 mmol, 1.0 equiv) in Ac₂O (15 mL) was stirred at room temperature for 3 h. The reaction mixture was concentrated and extracted with PE. The combined organic layer was dried and concentrated to yield N-(5-bromo-6-chloropyrazin-2-yl)acetamide as a yellow solid. LC/MS: mass calculated for C₆H₅BrClN₃O: 248.93, measured: 250.00 [M+H]⁺.

### Step 2: N-(6-Chloro-5-(1-ethoxyvinyl)pyrazin-2-yl)acetamide

To a solution of N-(5-bromo-6-chloropyrazin-2-yl)acetamide (1.3 g, 5.19 mmol, 1 equiv) and tributyl(1-ethoxyvinyl)tin (1.8 ml, 5.19 mmol, 1.0 equiv) in 1,4-dioxane (15 mL) was added Pd(PPh₃)₂Cl₂ (364 mg, 0.52 mmol, 0.1 equiv) under N₂(g). The reaction mixture was stirred at 90° C. for 3 h. The reaction mixture was diluted with water and extracted with ethyl acetate, the organic layer was washed with brine and dried over anhydrous Na₂SO₄. The solvent was removed under reduced pressure and the residue was purified by flash column chromatography on silica gel (EA/PE, 1→40%) to yield N-(6-chloro-5-(1-ethoxyvinyl)pyrazin-2-yl)acetamide as a yellow solid. LC/MS: mass calculated for C₁₀H₁₂ClN₃O₂: 241.06, measured: 242.05 [M+H]⁺.

### Step 3: N-(5-(2-Bromoacetyl)-6-chloropyrazin-2-yl)acetamide

To a solution of N-(6-chloro-5-(1-ethoxyvinyl)pyrazin-2-yl)acetamide (1.2 g, 4.96 mmol, 1.0 equiv) in THE (10 mL) and H₂O (2 mL) was added NBS (972 mg, 5.46 mmol, 1.1 equiv) at 0° C. The reaction mixture was stirred at room temperature for 30 min. The reaction mixture was diluted with water and extracted with ethyl acetate, the organic layer was washed with brine and dried over anhydrous Na₂SO₄, and concentrated. The residue was purified by flash column chromatography on silica gel (EA/PE, 1→40%) to yield N-(5-(2-bromoacetyl)-6-chloropyrazin-2-yl)acetamide as a yellow solid. LC/MS: mass calculated for C₈H₇BrClN₃O₂: 290.94, measured: 292.05 [M+H]⁺.

### Step 4: rac-2-(5-acetamido-3-chloropyrazin-2-yl)-2-oxoethyl (8R)-2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate

To a solution of (8R)-2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylic acid (0.3 g, 0.77 mmol, 1.00 equiv) and potassium carbonate (138 mg, 1.0 mmol, 1.30 equiv) in DMF (3.0 mL) was added N-(5-(2-bromoacetyl)-6-chloropyrazin-2-yl)acetamide (292 mg, 1.0 mmol, 1.30 equiv) and the solution was stirred for 1 h at room temperature. The reaction was concentrated and the residue was purified by silica gel chromatography (0-10% MeOH/DCM) to yield rac-2-(5-acetamido-3-chloropyrazin-2-yl)-2-oxoethyl (8R)-2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate as a light yellow solid.

### Step 5: N-(6-chloro-5-(2-((8R)-2-(3-chloro-2-fluoro-6-(1H-tetrazol-1-yl)phenyl)-8-methyl-4-oxo-4,6,7,8-tetrahydropyrrolo[1,2-a]pyrimidin-6-yl)-1H-imidazol-4-yl)pyrazin-2-yl)acetamide

A mixture of 2-(5-acetamido-3-chloropyrazin-2-yl)-2-oxoethyl (8R)-2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate (0.3 g, 0.50 mmol, 1.00 equiv), ammonium acetate (384 mg, 4.98 mmol, 10.0 equiv) and acetic acid (150 mg, 2.49 mmol, 5.00 equiv) in toluene (5.0 mL) was stirred for 2 h at 110° C. The reaction mixture was concentrated and the residue was purified by reverse phase chromatography on C18 (80 g, MeCN/H₂O (0.05% CF₃COOH): 0>>>50%) to yield N-(6-chloro-5-(2-((8R)-2-(3-chloro-2-fluoro-6-(1H-tetrazol-1-yl)phenyl)-8-methyl-4-oxo-4,6,7,8-tetrahydropyrrolo[1,2-a]pyrimidin-6-yl)-1H-imidazol-4-yl)pyrazin-2-yl)acetamide as a light yellow solid.

### Step 6: 6R,8R)-6-(4-(5-amino-3-chloropyrazin-2-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

To a solution of N-(6-chloro-5-(2-((8R)-2-(3-chloro-2-fluoro-6-(1H-tetrazol-1-yl)phenyl)-8-methyl-4-oxo-4,6,7,8-tetrahydropyrrolo[1,2-a]pyrimidin-6-yl)-1H-imidazol-4-yl)pyrazin-2-yl)acetamide (120 mg, 0.21 mmol, 1.00 equiv) in THE (2.0 mL) was added HCl solution (1.0 mL, 6.0 M) and the resulting mixture was stirred for 2 h at 60° C. The solution was concentrated and the residue was purified by silica gel chromatography (0-80% EtOAc/petroleum ether) and then Prep-HPLC to yield (6R,8R)-6-(4-(5-amino-3-chloropyrazin-2-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one as a white solid.

LC/MS mass calculated for: C₂₂H₁Cl₂FN₁₁O: 540.34, measured (ES, m/z): 540.05 [M+H]⁺. ¹H NMR: (DMSO, 400 MHz): δ 9.69 (s, 1H), 8.04 (t, J=8.2 Hz, 1H), 7.94-7.99 (m, 2H), 7.73 (dd, J=8.6, 1.5 Hz, 1H), 6.58 (d, J=2.1 Hz, 1H), 5.67 (t, J=8.5 Hz, 1H), 3.13-3.20 (m, 1H), 2.81-2.88 (m, 1H), 1.86-1.99 (m, 1H), 1.12 (d, J=7.0 Hz, 3H). ¹⁹F NMR: (DMSO, 376 MHz): δ −73.68, −113.46

### Example 94 (6*R,8R)-6-(4-(5-Amino-3-chloropyrazin-2-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

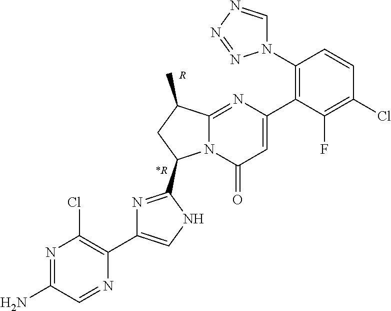

LC/MS mass calculated for: C₂₂H₁₆Cl₂FN₁₁O: 540.34, measured (ES, m/z): 540.05 [M+H]⁺. ¹H NMR (DMSO, 400 MHz): δ 9.71 (s, 1H), 7.99-8.08 (m, 1H), 7.94 (s, 1H), 7.73 (dd, J=8.7, 1.5 Hz, 2H), 6.65 (d, J=2.1 Hz, 1H), 5.79 (dd, J=9.2, 2.8 Hz, 1H), 3.30-3.43 (m, 1H), 2.39-2.48 (m, 1H), 2.16-2.26 (m, 1H), 0.94 (d, J=7.0 Hz, 3H). ¹⁹F NMR (DMSO, 376 MHz): δ −73.68, −113.64.

### Example 95 (6S)-6-(5-(5-Amino-3-methylpyrazin-2-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

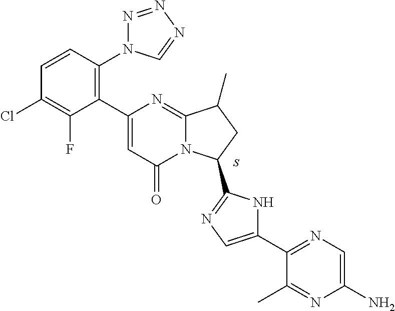

### Step 1: 1-(5-Amino-3-methylpyrazin-2-yl)ethan-1-one

To a solution of 5-bromo-6-methylpyrazin-2-amine (500 mg, 2.66 mmol) in 1,4-dioxane (10 mL) were added tributyl(1-ethoxyvinyl)stannane (1.2 g, 3.19 mmol), and Pd(PPh₃)₄ (307 mg, 0.26 mmol). The resulting mixture was maintained under nitrogen and stirred at 100° C. overnight. After cooling to room temperature, the reaction was quenched with water (20 mL). The resulting mixture was extracted with ethyl acetate (3×20 mL). The organic layers were combined, dried over anhydrous sodium sulfate, filtered and concentrated. The residue was purified by silica gel chromatography (0→50% ethyl acetate/petroleum ether) to yield the 1-(5-amino-3-methylpyrazin-2-yl)ethan-1-one as a yellow solid. LC/MS: mass calculated for C₇H₉N₃O: 151.07, measured: 152.10 [M+H]+

### Step 2: 1-(5-Amino-3-methylpyrazin-2-yl)-2-bromoethan-1-one

To a solution of 1-(5-amino-3-methylpyrazin-2-yl)ethan-1-one (150 mg, 0.99 mmol) and HBr (486 mg, 1.98 mmol) in AcOH (10 mL) was added pyridinium tribromide (285 mg, 0.89 mmol). The mixture was stirred at room temperature for 2 h, concentrated under reduced pressure to yield 1-(5-amino-3-methylpyrazin-2-yl)-2-bromoethan-1-one as a black solid. LC/MS: mass calculated for C₇H₉BrN₃O: 228.99, measured: 232.0 [M+2H+2]⁺.

### Step 3: 2-(5-amino-3-methylpyrazin-2-yl)-2-oxoethyl (6S,8S)-2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate

To a solution of (6S,8S)-2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylic acid (150 mg, 0.38 mmol) in DMF (10 mL) was added K₂CO₃ (80 mg, 0.58 mmol).

After 0.5h of stirring, 1-(5-amino-3-methylpyrazin-2-yl)-2-bromoethan-1-one (132 mg, 0.58 mmol) was added. The mixture was stirred at room temperature for 1 h, extracted with ethyl acetate (3×20 mL). The organic layers were combined, dried over anhydrous sodium sulfate, filtered and concentrated. The residue was purified by silica gel chromatography (0-20% MeOH/DCM) to yield the 2-(5-amino-3-methylpyrazin-2-yl)-2-oxoethyl (6S,8S)-2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate as a yellow oil.

### Step 4: (*R)-6-(5-(5-amino-3-methyl pyrazin-2-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one and (*S)-6-(5-(5-amino-3-methylpyrazin-2-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one 1-oxide

To a solution of 2-(5-amino-3-methylpyrazin-2-yl)-2-oxoethyl 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate (140 mg, 0.26 mmol) in toluene (10 mL) and glacial acetic acid (0.2 mL) was added ammonium acetate (400 mg, 5.19 mmol). The mixture stirred at 100° C. for 1h. After cooling to room temperature, the reaction was quenched with water (20 mL). The resulting mixture was extracted with ethyl acetate (3×20 mL). The organic layers were combined, dried over anhydrous sodium sulfate, filtered and concentrated. The residue was purified by silica gel chromatography (0-20% MeOH/DCM) to yield a second residue, which was purified by Chiral-HPLC with MtBE (0.1% DEA):EtOH=87:13 to yield (*R)-6-(5-(5-amino-3-methyl pyrazin-2-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one as a light yellow solid and (*S)-6-(5-(5-amino-3-methylpyrazin-2-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one 1-oxide as a light yellow solid.

LC/MS mass calculated for: C₂₃H₁₉ClFN₁₁O: 519.14, measured (ES, m/z): 520.15 [M+H]⁺. ¹H NMR (400 MHz, Methanol-d₄) δ 9.43-9.51 (m, 1H), 7.81-7.95 (m, 2H), 7.55-7.70 (m, 1H), 7.25 (s, 1H), 6.55-6.75 (m, 1H), 5.79-5.91 (m, 1H), 3.49-3.58 (m, 1H), 2.66-2.75 (m, 1H), 2.50 (s, 3H), 2.19-2.30 (m, 1H), 1.12 (d, J=7.0 Hz, 3H). ¹⁹F NMR (376 MHz, Methanol-d₄) δ −76.94, −114.52.

### Example 96 (6R)-6-(5-(5-Amino-3-methylpyrazin-2-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

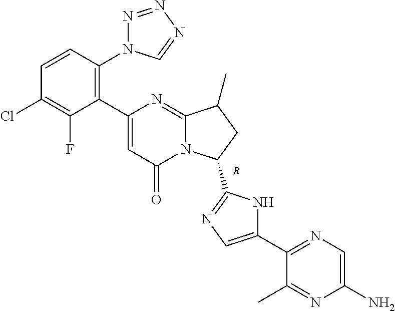

LC/MS mass calculated for: C₂₃H₁₉ClFN₁₁O: 519.14, measured (ES, m/z): 520.15 [M+H]+. ¹H NMR (400 MHz, Methanol-d₄) δ 9.49 (s, 1H), 7.81-7.91 (m, 1H), 7.86 (s, 1H), 7.54-7.69 (m, 1H), 7.25 (s, 1H), 6.55-6.75 (m, 1H), 5.79-5.91 (m, 1H), 3.49-3.58 (m, 1H), 2.66-2.75 (m, 1H), 2.50 (s, 3H), 2.19-2.30 (m, 1H), 1.12 (d, J=7.0 Hz, 3H). ¹⁹F NMR (376 MHz, Methanol-d₄) δ −76.96, −114.54.

### Example 97 (6*S,8*S)-2-(3-Chloro-2-fluoro-6-(1H-tetrazol-1-yl)phenyl)-6-(5-(2-methoxypyridin-4-yl)-1H-imidazol-2-yl)-8-methyl-7,8-dihydropyrrolo[1,2-a]pyrimidin-4(6H)-one

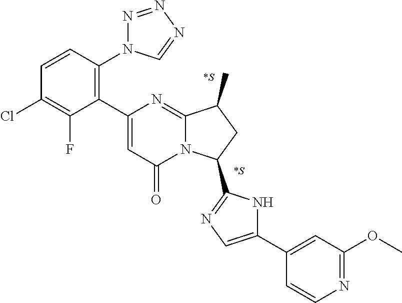

LC/MS mass calculated for: C₂₄H₁₉ClFN₉O₂: 519.13, measured (ES, m/z): 520.00 [M+H]⁺. ¹H NMR: (300 MHz, DMSO-d₆) δ 12.56 (s, 1H), 9.74 (s, 1H), 8.03-8.19 (m, 2H), 7.72-7.87 (m, 2H), 7.32-7.36 (m, 1H), 7.09 (s, 1H), 6.57 (d, J=2.1 Hz, 1H), 5.48-5.54 (m, 1H), 3.85 (s, 3H), 3.08-3.12 (m, 1H), 2.73-2.85 (m, 1H), 1.86-1.94 (m, 1H), 1.16 (d, J=7.2 Hz, 3H). ¹⁹F NMR: (282 MHz, DMSO-d₆) δ −113.78.

### Example 98 (6*S,8*R)-2-(3-Chloro-2-fluoro-6-(1H-tetrazol-1-yl)phenyl)-6-(5-(2-methoxypyridin-4-yl)-1H-imidazol-2-yl)-8-methyl-7,8-dihydropyrrolo[1,2-a]pyrimidin-4(6H)-one

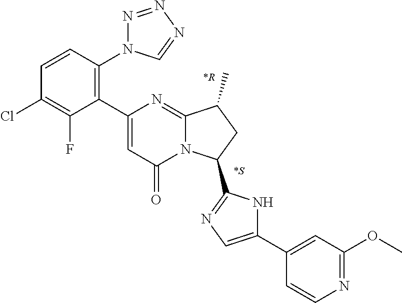

LC/MS mass calculated for: C₂₄H₁₉ClFN₉O₂: 519.13, measured (ES, m/z): 520.00 [M+H]⁺. ¹H NMR (300 MHz, DMSO-d₆) δ 12.54 (s, 1H), 9.77 (s, 1H), 8.01-8.12 (m, 2H), 7.73-7.86 (m, 2H), 7.27-7.29 (m, 1H), 7.06 (s, 1H), 6.64 (d, J=2.2 Hz, 1H), 5.62 (d, J=8.6 Hz, 1H), 3.84 (s, 3H), 3.46-3.53 (m, 1H), 2.39-2.44 (m, 1H), 2.13-2.26 (m, 1H), 0.98 (d, J=6.9 Hz, 3H). ¹⁹F NMR: (282 MHz, DMSO-d₆) δ −113.71.

### Example 99 (6*R,8*R)-2-(3-Chloro-2-fluoro-6-(1H-tetrazol-1-yl)phenyl)-6-(5-(2-methoxypyridin-4-yl)-1H-imidazol-2-yl)-8-methyl-7,8-dihydropyrrolo[1,2-a]pyrimidin-4(6H)-one

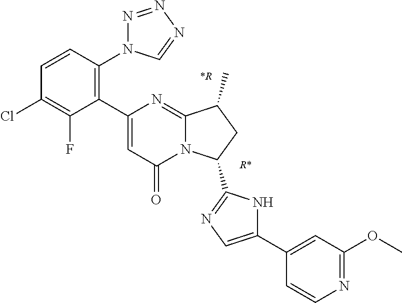

LC/MS mass calculated for: C₂₄H₁₉ClFN₉O₂:519.13, measured (ES, m/z): 520.00 [M+H]⁺. ¹H NMR (300 MHz, DMSO-d₆) δ 12.56 (s, 1H), 9.77 (s, 1H), 8.00-8.13 (m, 2H), 7.83 (d, J=1.8 Hz, 1H), 7.76-7.79 (m, 1H), 7.28-7.32 (m, 1H), 7.09 (s, 1H), 6.56 (d, J=2.1 Hz, 1H), 5.47-5.56 (m, 1H), 3.85 (s, 3H), 3.07-3.12 (m, 1H), 2.74-2.82 (m, 1H), 1.88-1.92 (m, 1H), 1.16 (d, J=7.2 Hz, 3H). ¹⁹F NMR: (282 MHz, DMSO-d₆) δ −113.78.

### Example 100 (6*R,8*S)-2-(3-Chloro-2-fluoro-6-(1H-tetrazol-1-yl)phenyl)-6-(5-(2-methoxypyridin-4-yl)-1H-imidazol-2-yl)-8-methyl-7,8-dihydropyrrolo[1,2-a]pyrimidin-4(6H)-one

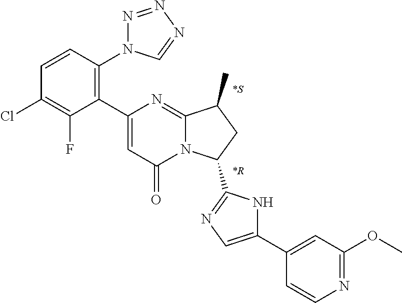

LC/MS mass calculated for: C₂₄H₁₉ClFN₉O₂:519.13, measured (ES, m/z): 520.00 [M+H]+. ¹H NMR (300 MHz, DMSO-d₆) δ 12.54 (s, 1H), 9.77 (s, 1H), 8.01-8.19 (m, 2H), 7.73-7.86 (m, 2H), 7.28-7.31 (m, 1H), 7.06 (d, J=1.5 Hz, 1H), 6.64 (d, J=2.1 Hz, 1H), 5.62 (d, J=8.6 Hz, 1H), 3.84 (s, 3H), 3.46-3.56 (m, 1H), 2.36-2.46 (m, 1H), 2.09-2.26 (m, 1H), 0.98 (d, J=6.9 Hz, 3H). ¹⁹F NMR: (282 MHz, DMSO-d₆) δ −113.71.

### Example 101 7-(2-((6S,8R)-2-(3-Chloro-2-fluoro-6-(1H-tetrazol-1-yl)phenyl)-8-methyl-4-oxo-4,6,7,8-tetrahydropyrrolo[1,2-a]pyrimidin-6-yl)-1H-imidazol-5-yl)quinolin-4(1H)-one

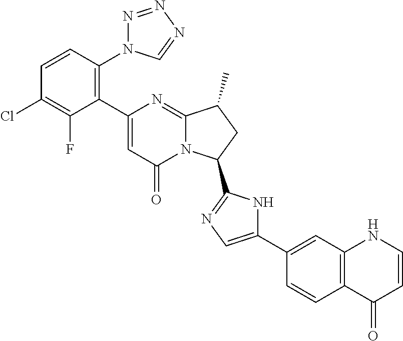 [SMILES: C[C@@H]1C[C@@H](c2ncc(-c3ccc4c(=O)cc[nH]c4c3)[nH]2)n2c1nc(-c1c(-n3cnnn3)ccc(Cl)c1F)cc2=O]

LC/MS mass calculated for: C₂₇H₁₉ClFN₉O₂:555.13, measured (ES, m/z): 556.10 [M+H]⁺. ¹H NMR (400 MHz, Methanol-d₄) δ 9.50 (s, 1H), 8.20 (d, J=8.6 Hz, 1H), 7.86-7.98 (m, 3H), 7.73 (d, J=8.6 Hz, 1H), 7.61 (dd, J=8.7, 1.6 Hz, 2H), 6.64 (dd, J=2.2, 0.8 Hz, 1H), 6.30 (d, J=7.3 Hz, 1H), 5.76 (dd, J=9.1, 1.6 Hz, 1H), 3.56-3.65 (m, 1H), 2.58-2.68 (m, 1H), 2.21-2.34 (m, 1H), 1.13 (d, J=7.0 Hz, 3H). ¹⁹F NMR (376 MHz, Methanol-d₄) δ −117.28

### Example 102 7-(2-((6*S,8*S)-2-(3-Chloro-2-fluoro-6-(1H-tetrazol-1-yl)phenyl)-8-methyl-4-oxo-4,6,7,8-tetrahydropyrrolo[1,2-a]pyrimidin-6-yl)-1H-imidazol-5-yl)quinolin-4(1H)-one

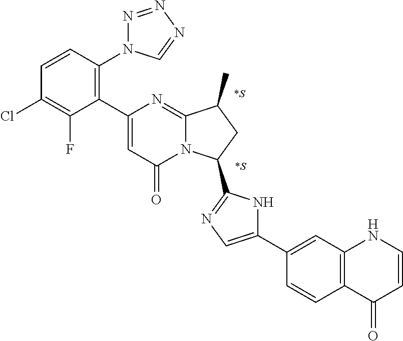 [SMILES: C[C@H]1C[C@@H](c2ncc(-c3ccc4c(=O)cc[nH]c4c3)[nH]2)n2c1nc(-c1c(-n3cnnn3)ccc(Cl)c1F)cc2=O]

LC/MS mass calculated for: C₂₇H₁₉ClFN₉O₂:555.13, measured (ES, m/z): 556.10 [M+H]⁺. ¹H NMR (400 MHz, Methanol-d₄) δ 9.49 (s, 1H), 8.22 (d, J=8.7 Hz, 1H), 7.87-7.89 (m, 3H), 7.76 (d, J=8.6 Hz, 1H), 7.60-7.68 (m, 2H), 6.56-6.61 (m, 1H), 6.31 (d, J=7.3 Hz, 1H), 5.60 (t, J=8.3, 7.4 Hz, 1H), 3.13-3.24 (m, 1H), 2.83-2.95 (m, 1H), 2.06-2.17 (m, 1H), 1.30 (d, J=7.1 Hz, 3H). ¹⁹F NMR (376 MHz, Methanol-d₄) δ −114.51.

### Example 103 (6S,8S)-2-(3-chloro-2-fluoro-6-(1H-tetrazol-1-yl)phenyl)-6-(5-(2-methoxypyrimidin-4-yl)-1H-imidazol-2-yl)-8-methyl-7,8-dihydropyrrolo[1,2-a]pyrimidin-4(6H)-one

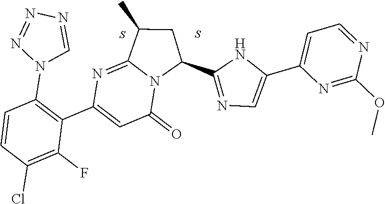

LC/MS mass calculated for: C₂₃H₁₈ClFN₁₀O₂: 520.13, measured (ES, m/z): 521.20 [M+H]⁺. ¹H NMR: (300 MHz, DMSO-d₆) δ 9.75 (s, 1H), 8.56 (d, J=5.2 Hz, 1H), 8.07 (dd, J=8.7, 7.7 Hz, 1H), 7.98 (s, 1H), 7.77 (dd, J=8.7, 1.5 Hz, 1H), 7.48 (d, J=5.1 Hz, 1H), 6.58 (d, J=2.1 Hz, 1H), 5.58 (t, J=7.5 Hz, 1H), 3.95 (s, 3H), 3.05-3.19 (m, 1H), 2.74-2.87 (m, 1H), 1.81-1.95 (m, 1H), 1.13 (d, J=7.1 Hz, 3H).

### Example 104 (6*R,8*S)-2-(3-Chloro-2-fluoro-6-(1H-tetrazol-1-yl)phenyl)-6-(5-(2-methoxypyrimidin-4-yl)-1H-imidazol-2-yl)-8-methyl-7,8-dihydropyrrolo[1,2-a]pyrimidin-4(6H)-one

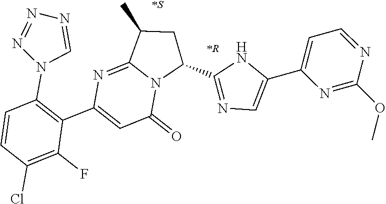

LC/MS mass calculated for: C₂₃H₁₈ClFN₁₀O₂: 520.13, measured (ES, m/z): 521.20 [M+H]⁺. ¹H NMR: (300 MHz, DMSO-d₆) δ 9.77 (s, 1H), 8.53 (d, J=5.2 Hz, 1H), 8.07 (dd, J=8.7, 7.7 Hz, 1H), 7.92 (s, 1H), 7.78 (dd, J=8.7, 1.5 Hz, 1H), 7.44 (d, J=5.2 Hz, 1H), 6.65 (d, J=2.1 Hz, 1H), 5.64-5.73 (m, 1H), 3.94 (s, 3H), 3.35-3.49 (m, 1H), 2.35-2.45 (m, 1H), 2.12-2.29 (m, 1H), 0.98 (d, J=6.9 Hz, 3H). ¹⁹F NMR: (282 MHz, DMSO-d₆) −74.55, −113.72.

### Example 105 (6*R,8*R)-6-(5-(1,2,3-Thiadiazol-5-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

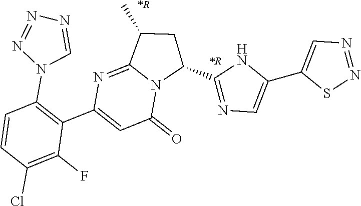

LC/MS mass calculated for: C₂₀H₁₄ClFN₁₀OS: 496.07, (ES, m/z): 497.15 [M+H]⁺. ¹H NMR (300 MHz, Methanol-d₄) δ 9.56 (d, J=5.2 Hz, 1H), 9.28 (d, J=0.9 Hz, 1H), 8.23 (s, 1H), 7.95 (dd, J=8.7, 7.6 Hz, 1H), 7.66 (dd, J=8.7, 1.6 Hz, 1H), 6.73 (d, J=2.4 Hz, 1H), 6.01-6.06 (m, 1H), 3.49-3.65 (m, 1H), 2.70-2.80 (m, 1H), 2.51-2.60 (m, 1H), 1.15 (d, J=7.0 Hz, 3H). ¹⁹F NMR (376 MHz, Methanol-d₄) −79.81, 114.32.

### Example 106 (6*R,8*S)-6-(5-(1,2,3-Thiadiazol-5-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

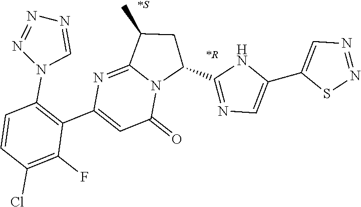

LC/MS mass calculated for: C₂₀H₁₄ClFN₁₀OS: 496.07, (ES, m/z): 497.15 [M+H]⁺. ¹H NMR (300 MHz, methanol-d₄) δ 9.53 (s, 1H), 9.23 (s, 1H), 8.22 (s, 1H), 7.95 (dd, J=8.7, 7.6 Hz, 1H), 7.65 (dd, J=8.7, 1.6 Hz, 1H), 6.74 (dd, J=2.5, 0.7 Hz, 1H), 6.01 (dd, J=9.7, 3.6 Hz, 1H), 3.50-3.61 (m, 1H), 2.65-2.80 (m, 1H), 2.50-2.61 (m, 1H), 1.15 (d, J=7.1 Hz, 3H). ¹⁹F NMR: (376 MHz, DMSO-d₆) −79.81, 114.32.

### Example 107 (6*S,8*S)-6-(5-(1H-Pyrazol-4-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

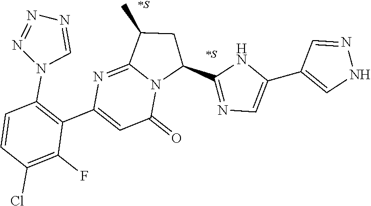

### Step 1:2-Chloro-1-(1-(tetrahydro-2H-pyran-2-yl)-1H-pyrazol-4-yl)ethan-1-one

4-Iodo-1-(tetrahydro-2H-pyran-2-yl)-1H-pyrazole (700 mg, 2.51 mmol) was dissolved in THE (10 mL) and cooled in an ice bath. Isopropyl-magnesium chloride (1.26 mL, 2.51 mmol) was added slowly. The reaction mixture was stirred for 10 minutes, and then 2-chloro-N-methoxy-N-methylacetamide (346 mg, 2.52 mmol) in THE (5 mL) was added slowly by syringe. The reaction mixture was warmed to room temperature and stirred for 1 h. The reaction mixture was partitioned between EtOAc and 1 M HCl, and the organic layer was dried over sodium sulfate, filtered and concentrated and purified by silica gel chromatography (0→30% DCM/MeOH) to yield 2-chloro-1-(1-(tetrahydro-2H-pyran-2-yl)-1H-pyrazol-4-yl)ethan-1-one as colorless oil. LC/MS: mass calculated for C₁₀H₁₃ClN₂O₂: 228.07, measured: 229.15 [M+H]⁺.

### Step 2: 2-oxo-2-(1-(tetrahydro-2H-pyran-2-yl)-1H-pyrazol-4-yl)ethyl 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate

To a solution of 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylic acid (150 mg, 0.38 mmol, 1 equiv) in N,N-dimethylformamide (2 mL) was added potassium carbonate (159 mg, 1.15 mmol, 3 equiv). After the reaction mixture was stirred at room temperature 30 min, 2-bromo-1-(1,2,3-thiadiazol-5-yl)ethan-1-one (176 mg, 0.77 mmol, 2 equiv) was added. The mixture was stirred at room temperature for 2 h. The residue was purified by reverse column chromatography with CH₃CN/0.05% TFA water (5%-80%) to yield 2-oxo-2-(1-(tetrahydro-2H-pyran-2-yl)-1H-pyrazol-4-yl)ethyl 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate as an light yellow solid. LC/MS: mass calculated for C₂₆H₂₄ClFN₈O₅: 582.15, measured: 583.25 [M+H]⁺.

### Step 3: (6S)-2-(3-chloro-2-fluoro-6-(1H-tetrazol-1-yl)phenyl)-8-methyl-6-(5-(1-(tetrahydro-2H-pyran-2-yl)-1H-pyrazol-4-yl)-1H-imidazol-2-yl)-7,8-dihydropyrrolo[1,2-a]pyrimidin-4(6H)-one

To a solution of 2-oxo-2-(1-(tetrahydro-2H-pyran-2-yl)-1H-pyrazol-4-yl)ethyl 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate (150 mg, 0.26 mmol, 1 eq) in toluene (20 mL) were added ammonium acetate (298 mg, 3.86 mmol, 15 eq) and acetic acid (155 mg, 2.57 mmol, 10 eq). The reaction mixture was stirred at 100° C. for 1 h and concentrated under reduced pressure. The residue was purified by silica gel chromatography (0-10% MeOH/DCM) to yield 2-(3-chloro-2-fluoro-6-(1H-tetrazol-1-yl)phenyl)-8-methyl-6-(5-(1-(tetrahydro-2H-pyran-2-yl)-1H-pyrazol-4-yl)-1H-imidazol-2-yl)-7,8-dihydropyrrolo[1,2-a]pyrimidin-4(6H)-one as a light yellow solid. LC/MS: mass calculated for C₂₆H₂₄ClFN₁₀O₂: 562.18, measured: 563.25 [M+H]+.

### Step 4: (6*S,8*S)-6-(5-(1H-Pyrazol-4-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

To a solution of 6-(5-(1H-pyrazol-4-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one (80 mg, 0.142 mmol) in methanol (2 mL) was added hydrochloric acid (2 mL, 4 M). The mixture was stirred 2 h at room temperature. The mixture was concentrated and the residue was purified by prep-chiral-HPLC with MtBE (0.1% DEA):EtOH=75:25 to yield (6S,8S)-6-(5-(1H-pyrazol-4-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one as an off-white solid and (6R,8R)-6-(5-(1H-pyrazol-4-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one as an off-white solid and (6R,8S)-6-(5-(1H-pyrazol-4-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one as an light yellow solid and (6S,8R)-6-(5-(1H-pyrazol-4-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one as an off-white solid.

LC/MS mass calculated for: C₂₁H₁₆ClFN₁₀O: 478.12, (ES, m/z): 479.25 [M+H]⁺. ¹H-NMR (300 MHz, DMSO-d₆) δ 12.74 (s, 1H), 12.10 (s, 1H), 9.74 (s, 1H), 8.06 (dd, J=8.7, 7.7 Hz, 1H), 7.74-7.95 (m, 3H), 7.17 (s, 1H), 6.55 (d, J=2.1 Hz, 1H), 5.47 (dd, J=8.5, 6.3 Hz, 1H), 3.0-3.15 (m, 1H), 2.66-2.82 (m, 1H), 1.76-1.93 (m, 1H), 1.13 (d, J=7.1 Hz, 3H). ¹⁹F NMR (282 MHz, DMSO-d₆) −73.40, −113.73.

### Example 108 (6*R,8*R)-6-(5-(1H-Pyrazol-4-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

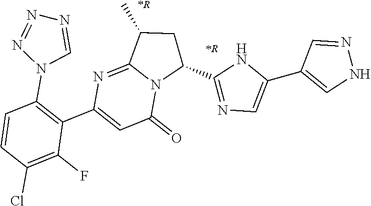

LC/MS mass calculated for: C₂₁H₁₆ClFN₁₀O: 478.12, (ES, m/z): 479.25 [M+H]⁺. ¹H-NMR: (300 MHz, DMSO-d₆) δ 12.60-13.00 (m, 1H), 12.1-12.5 (m, 1H), 9.74 (d, J=10.1 Hz, 1H), 8.00-8.11 (m, 1H), 7.60-7.90 (m, 3H), 6.84-7.29 (m, 1H), 6.54 (s, 1H), 5.47 (dd, J=8.6, 6.0 Hz, 1H), 3.0-3.15 (m, 1H), 2.66-2.82 (m, 1H), 1.76-1.93 (m, 1H), 1.13 (d, J=7.1 Hz, 3H). ¹⁹F NMR: (282 MHz, DMSO-d₆) −73.40, −113.77

### Example 109 (6*R,8*S)-6-(5-(1H-Pyrazol-4-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

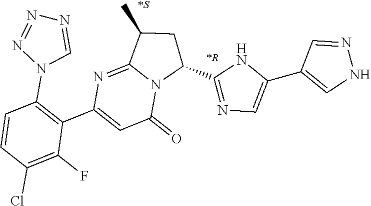

LC/MS mass calculated for: C₂₁H₁₆ClFN₁₀O: 478.12, (ES, m/z): 479.20 [M+H]⁺. ¹H NMR: (300 MHz, DMSO-d₆) δ 12.79 (s, 1H), 12.11 (s, 1H), 9.77 (s, 1H), 8.06 (dd, J=8.7, 7.7 Hz, 1H), 7.57-7.79 (m, 3H), 7.10 (s, 1H), 6.63 (d, J=2.1 Hz, 1H), 5.54-5.63 (m, 1H), 3.46-3.55 (m, 1H), 2.39-2.50 (m, 1H), 2.07-2.24 (m, 1H), 0.96 (d, J=7.0 Hz, 3H). ¹⁹F NMR: (282 MHz, DMSO-d₆) −73.42, −113.72.

### Example 110 (6*S)-6-(5-(1,2,3-Thiadiazol-5-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

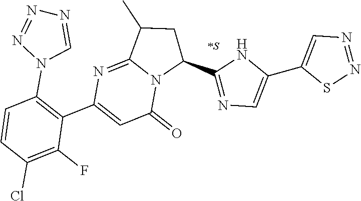

### Step 1: 2-Bromo-1-(1,2,3-thiadiazol-5-yl)ethan-1-one

To a solution of 1-(1,2,3-thiadiazol-5-yl)ethan-1-one (400 mg, 3.12 mmol, 1.0 eq) in acetic acid (20 mL) were added hydrogen bromide (2.3 g, 9.36 mmol, 3.0 eq) and pyridinium tribromide (898 mg, 2.81 mmol, 1.0 eq). The reaction mixture was stirred 1 h at room temperature. The reaction mixture was filtered, and the solid was collected and dried in vacuo to yield 2-bromo-1-(1,2,3-thiadiazol-5-yl)ethan-1-one as a light yellow solid, which was used in the next step without further purification.

### Step 2: 2-oxo-2-(1,2,3-thiadiazol-5-yl)ethyl 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate

To a solution of 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylic acid (200 mg, 0.51 mmol, 1 equiv) in N,N-dimethylformamide (3 mL) was added cesium carbonate (167 mg, 0.51 mmol, 1 equiv). After the reaction mixture was stirred at room temperature 30 min, 2-bromo-1-(1,2,3-thiadiazol-5-yl)ethan-1-one (159 mg, 0.77 mmol, 1.5 equiv) was added. The mixture was stirred at room temperature for 2 h and then concentrated under reduced pressure. The residue was purified by silica gel chromatography (0-10% MeOH/DCM) to yield 2-oxo-2-(1,2,3-thiadiazol-5-yl)ethyl 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate as a red solid. LC/MS: mass calculated for C₂₀H₁₄ClFN₈O₄S: 516.05, measured: 535.00 [M+H+18]⁺.

### Step 3: (6*S)-6-(5-(1,2,3-Thiadiazol-5-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

To a solution of 2-oxo-2-(1,2,3-thiadiazol-5-yl)ethyl 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate (150 mg, 0.290 mmol, 1 eq) in toluene (20 mL) were added ammonium acetate (335.535 mg, 4.353 mmol, 15 eq) and acetic acid (174.268 mg, 2.902 mmol, 10 eq). The reaction mixture was stirred at 100° C. for 1 h. The residue was purified by reverse column chromatography on C18 column with CH₃CN/0.05% TFA water (5%-80%) to yield (6S)-6-(5-(1,2,3-thiadiazol-5-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one as an light yellow solid and a racemic mixture of (6R)-6-(5-(1,2,3-thiadiazol-5-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one as an light yellow solid. The racemic mixture was further purified by prep-Chiral-HPLC with MtBE (0.1% DEA):EtOH=75:25 to yield (6R,8R)-6-(5-(1,2,3-thiadiazol-5-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one as a light yellow solid and (6R,8S)-6-(5-(1,2,3-thiadiazol-5-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one as an off-white solid.

LC/MS mass calculated for: C₂₀H₁₄ClFN₁₀OS: 496.07, (ES, m/z): 497.20 [M+H]⁺. ¹H NMR (400 MHz, Methanol-d₄) δ 9.47 (s, 1H), 9.04 (s, 1H), 7.92 (dd, J=8.7, 7.5 Hz, 1H), 7.75 (s, 1H), 7.63 (dd, J=8.7, 1.6 Hz, 1H), 6.59 (dd, J=2.3, 0.7 Hz, 1H), 5.58 (t, J=8.0 Hz, 1H), 3.11-3.23 (m, 1H), 2.89-2.90 (m, 1H), 2.04-2.11 (m, 1H), 1.28 (d, J=7.1 Hz, 3H). ¹⁹F NMR (376 MHz, Methanol-d₄) −77.08, 114.54.

### Example 111 (6*S,8*R)-6-(5-(1H-Pyrazol-4-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

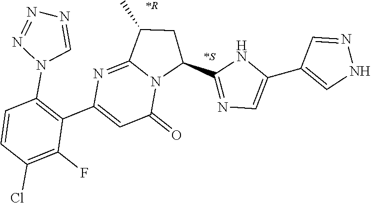

LC/MS mass calculated for: C₂₁H₁₆ClFN₁₀O: 478.12, (ES, m/z): 479.25 [M+H]⁺. ¹H NMR: (300 MHz, DMSO-d₆) δ 12.75 (brs, 1H), 12.09 (brs, 1H), 9.76 (s, 1H), 8.01-8.12 (m, 1H), 7.60-7.95 (m, 3H), 6.98-7.29 (brs, 1H), 6.63 (d, J=2.1 Hz, 1H), 5.59 (d, J=8.6 Hz, 1H), 3.46-3.55 (m, 1H), 2.39-2.55 (m, 1H), 2.16-2.32 (m, 1H), 0.96 (d, J=7.0 Hz, 3H). ¹⁹F NMR: (282 MHz, DMSO-d₆) −73.41, −113.73.

### Example 112: (6*R,8*R)-2-(3-Chloro-2-fluoro-6-(1H-tetrazol-1-yl)phenyl)-8-methyl-6-(5-(2-oxoindolin-5-yl)-1H-imidazol-2-yl)-7,8-dihydropyrrolo[1,2-a]pyrimidin-4(6H)-one

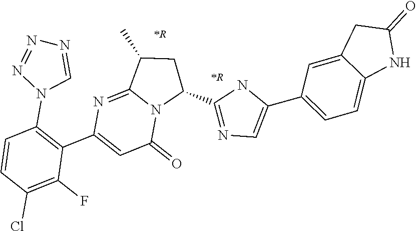

LC/MS: mass calculated for C₂₆H₁₉ClFN₉O₂: 543.13, measured (ES, m/z): 544.10 [M+H]⁺. ¹H NMR (300 MHz, Methanol-d₄) δ 9.50 (s, 1H), 7.92 (dd, J=8.7, 7.5 Hz, 1H), 7.49-7.67 (m, 3H), 7.26 (s, 1H), 6.91 (d, J=8.1 Hz, 1H), 6.65 (dd, J=2.4, 0.8 Hz, 1H), 5.74 (dd, J=9.2, 1.8 Hz, 1H), 3.55-3.61 (m, 3H), 2.54-2.68 (m, 1H), 2.18-2.35 (m, 1H), 1.12 (d, J=7.0 Hz, 3H). ¹⁹F NMR (282 MHz, Methanol-d₄) δ −114.54.

### Example 113: (6*S,8*S)-2-(3-Chloro-2-fluoro-6-(1H-tetrazol-1-yl)phenyl)-8-methyl-6-(5-(2-oxoindolin-5-yl)-1H-imidazol-2-yl)-7,8-dihydropyrrolo[1,2-a]pyrimidin-4(6H)-one

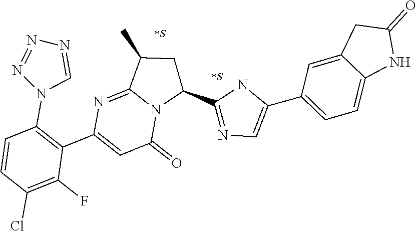

### Step 1: 2-oxo-2-(2-oxoindolin-5-yl)ethyl (6S,8S)-2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate

To a solution of rac-(6R,8R)-2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylic acid (10 mg, 0.03 mmol, 1 equiv) in CH₃CN (1 mL) was added K₂CO₃ (11 mg, 0.08 mmol, 3 equiv) under N₂. The reaction mixture was stirred for 0.5 h, then N-(5-(2-bromoacetyl) pyridin-2-yl)cyclopropanecarboxamide (11 mg, 0.05 mmol, 1.3 equiv) was added. The reaction mixture was stirred 1 h at room temperature, then concentrated under vacuum. The residue was purified by reverse-phase chromatography (C18 column, 330 g, CH₃CN/H₂O (0.05% TFA)=10%-70%) to yield rac-2-oxo-2-(2-oxoindolin-5-yl)ethyl (6R,8R)-2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate.

### Step 2: (6*S,8*S)-2-(3-Chloro-2-fluoro-6-(1H-tetrazol-1-yl)phenyl)-8-methyl-6-(5-(2-oxoindolin-5-yl)-1H-imidazol-2-yl)-7,8-dihydropyrrolo[1,2-a]pyrimidin-4(6H)-one

To a solution of rac-2-oxo-2-(2-oxoindolin-5-yl)ethyl (6R,8R)-2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate (80 mg, 0.14 mmol, 1 equiv) and ammonium acetate (219 mg, 2.85 mol, 20 equiv) in toluene (5 mL) was added AcOH (1 ml). The reaction mixture was stirred 2 h at 90° C., then cooled to room temperature and quenched with water, extracted with ethyl acetate, washed with brine, dried over Na₂SO₄ and concentrated under vacuum. The residue was purified by silica gel chromatography with MeOH/DCM (0-10%) to yield 2-(3-chloro-2-fluoro-6-(1H-tetrazol-1-yl)phenyl)-8-methyl-6-(5-(2-oxoindolin-6-yl)-1H-imidazol-2-yl)-7,8-dihydropyrrolo[1,2-a]pyrimidin-4(6H)-one as yellow oil, which was further purified by prep-HPLC and prep-chiral-HPLC to yield rac-(6R,8R)-2-(3-chloro-2-fluoro-6-(1H-tetrazol-1-yl)phenyl)-8-methyl-6-(5-(2-oxoindolin-6-yl)-1H-imidazol-2-yl)-7,8-dihydropyrrolo[1,2-a]pyrimidin-4(6H)-one.

LC/MS: mass calculated for C₂₆H₁₉ClFN₉O₂: 543.13, measured (ES, m/z): 544.10 [M+H]⁺. ¹H NMR (300 MHz, Methanol-d₄) δ 9.48 (s, 1H), 7.92 (dd, J=8.7, 7.6 Hz, 1H), 7.50-7.70 (m, 3H), 7.20-7.38 (m, 1H), 6.92 (d, J=8.1 Hz, 1H), 6.54-6.61 (m, 1H), 5.56 (t, J=8.1 Hz, 1H), 3.33-3.46 (m, 2H), 3.09-3.23 (m, 1H), 2.79-2.95 (m, 1H), 2.03-2.27 (m, 1H), 1.24-1.38 (m, 3H). ¹⁹F NMR (282 MHz, Methanol-d₄) δ −114.44.

### Example 114: (6*R,8*R)-6-(5-(2-Aminopyrimidin-5-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

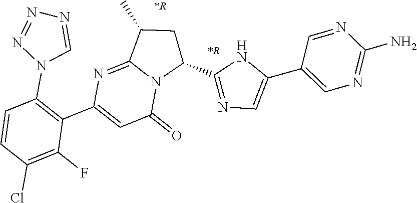

LC/MS: mass calculated for C₂₂H₁₇ClFN₁₁O: 505.13, measured (ES, m/z): 506.05 [M+H]⁺. ¹H NMR (400 MHz, Methanol-d₄) δ 9.47 (s, 1H), 8.55-8.65 (m, 2H), 7.92 (dd, J=8.7, 7.5 Hz, 1H), 7.63 (dd, J=8.7, 1.6 Hz, 1H), 7.40 (s, 1H), 6.58 (d, J=2.3 Hz, 1H), 5.56 (t, J=8.1 Hz, 1H), 3.09-3.21 (m, 1H), 2.81-2.93 (m, 1H), 2.04-2.16 (m, 1H), 1.27 (d, J=7.0 Hz, 3H). ¹⁹F NMR (376 MHz, Methanol-d₄) δ −114.62.

### Example 115 methyl (5-(2-((6R*,8S*)-2-(3-Chloro-2-fluoro-6-(1H-tetrazol-1-yl)phenyl)-8-methyl-4-oxo-4,6,7,8-tetrahydropyrrolo[1,2-a]pyrimidin-6-yl)-1H-imidazol-5-yl)pyridin-2-yl)carbamate

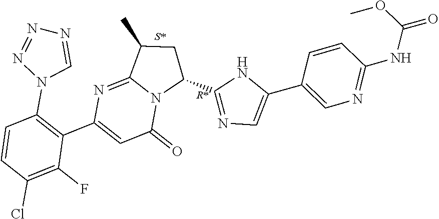

LC/MS mass calculated for: C₂₅H₂₀ClFN₁₀O₃: 562.14, (ES, m/z): 563.10 [M+H]⁺. ¹H NMR (300 MHz, Methanol-d₄) δ 9.48 (s, 1H), 8.61 (s, 1H), 8.06-8.09 (m, 1H), 7.89-7.94 (m, 2H), 7.49-7.68 (m, 2H), 6.92 (d, J=8.1 Hz, 1H), 5.56 (t, J=8.1 Hz, 1H), 3.79 (s, 3H), 3.09-3.23 (m, 1H), 2.79-2.95 (m, 1H), 2.03-2.27 (m, 1H), 1.28 (d, J=6.9 Hz, 3H). ¹⁹F NMR (282 MHz, Methanol-d₄) δ −114.47.

### Example 116 Methyl (5-(2-((6*S,8*S)-2-(3-Chloro-2-fluoro-6-(1H-tetrazol-1-yl)phenyl)-8-methyl-4-oxo-4,6,7,8-tetrahydropyrrolo[1,2-a]pyrimidin-6-yl)-1H-imidazol-5-yl)pyridin-2-yl)carbamate

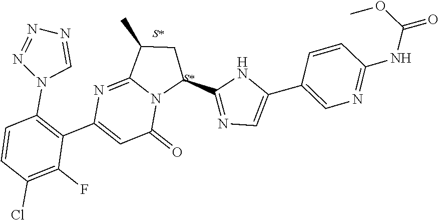

LC/MS mass calculated for: C₂₅H₂₀ClFN₁₀O₃: 562.14, (ES, m/z): 563.10 [M+H]⁺. ¹H NMR (300 MHz, Methanol-d₄) δ 9.48 (s, 1H), 8.61 (s, 1H), 8.04-8.10 (m, 1H), 7.92 (dd, J=8.7, 7.5 Hz, 2H), 7.63 (dd, J=8.7, 1.6 Hz, 1H), 7.48 (s, 1H), 6.58 (d, J=2.3 Hz, 1H), 5.57 (t, J=8.1 Hz, 1H), 3.79 (s, 3H), 3.10-3.24 (m, 1H), 2.80-2.96 (m, 1H), 2.21-2.02 (m, 1H), 1.28 (d, J=7.0 Hz, 3H). ¹⁹F NMR (282 MHz, Methanol-d₄) δ −114.47.

### Example 117 Methyl (5-(2-((6*R,8*R)-2-(3-Chloro-2-fluoro-6-(1H-tetrazol-1-yl)phenyl)-8-methyl-4-oxo-4,6,7,8-tetrahydropyrrolo[1,2-a]pyrimidin-6-yl)-1H-imidazol-5-yl)pyridin-2-yl)carbamate

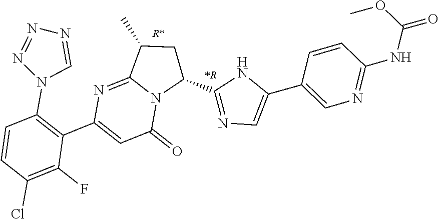

LC/MS mass calculated for: C₂₅H₂₀ClFN₁₀O₃: 562.14, (ES, m/z): 563.1 [M+H]⁺. ¹H NMR (300 MHz, Methanol-d₄) δ 9.49 (s, 1H), 8.58 (s, 1H), 8.01-8.07 (m, 1H), 7.87-7.98 (m, 2H), 7.63 (dd, J=8.7, 1.7 Hz, 1H), 7.45 (s, 1H), 6.65 (dd, J=2.4, 0.8 Hz, 1H), 5.75 (dd, J=9.1, 1.8 Hz, 1H), 3.79 (s, 3H), 3.54-3.65 (m, 1H), 2.60-2.66 (m, 1H), 2.16-2.36 (m, 1H), 1.12 (d, J=7.0 Hz, 3H). ¹⁹F NMR (282 MHz, Methanol-d₄) δ −114.59.

### Example 118 Methyl (5-(2-((6*S,8*R)-2-(3-Chloro-2-fluoro-6-(1H-tetrazol-1-yl)phenyl)-8-methyl-4-oxo-4,6,7,8-tetrahydropyrrolo[1,2-a]pyrimidin-6-yl)-1H-imidazol-5-yl)pyridin-2-yl)carbamate

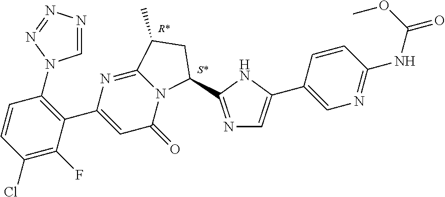 [SMILES: COC(=O)Nc1ccc(-c2cnc([C@@H]3C[C@@H](C)c4nc(-c5c(-n6cnnn6)ccc(Cl)c5F)cc(=O)n43)[nH]2)cn1]

LC/MS mass calculated for: C₂₅H₂₀ClFN₁₀O₃: 562.14, (ES, m/z): 563.10 [M+H]⁺. ¹H NMR (300 MHz, Methanol-d₄) δ 9.49 (s, 1H), 8.58 (s, 1H), 8.02-8.08 (m, 1H), 7.87-7.98 (m, 2H), 7.63 (dd, J=8.7, 1.6 Hz, 1H), 7.46 (s, 1H), 6.65 (d, J=2.5 Hz, 1H), 5.75 (d, J=8.8 Hz, 1H), 3.79 (s, 3H), 3.33-3.56 (m, 1H), 2.22-2.36 (m, 1H), 1.12 (d, J=7.0 Hz, 3H). ¹⁹F NMR (282 MHz, Methanol-d₄) δ −114.59.

### Example 119: (6*S,8*S)-6-(5-(2-Aminopyrimidin-5-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

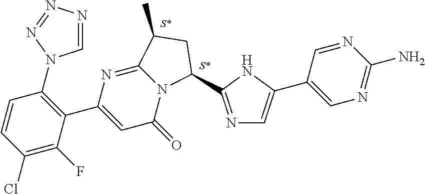 [SMILES: C[C@H]1C[C@@H](c2ncc(-c3cnc(N)nc3)[nH]2)n2c1nc(-c1c(-n3cnnn3)ccc(Cl)c1F)cc2=O]

### Step 1: 2-(2-aminopyrimidin-5-yl)-2-oxoethyl 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate

To a solution of 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylic acid (0.1 g, 0.26 mmol, 1 equiv) and in CH₃CN (5 mL) was added K₂CO₃ (106 mg, 0.77 mmol, 3 equiv) under N₂. The reaction mixture was stirred for 0.5 h, then N-(5-(2-bromoacetyl)-6-fluoropyridin-2-yl)acetamide (72 mg, 0.33 mmol, 1.3 equiv) was added. The reaction mixture was stirred 1 h at room temperature, then cooled to room temperature and quenched with water, extracted with ethyl acetate, washed with brine, dried over Na₂SO₄ and concentrated under vacuum. The residue was purified by silica gel chromatography with DCM/DCM (0-15%) to yield 2-(2-aminopyrimidin-5-yl)-2-oxoethyl (6R,8R)-2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate.

### Step 2: (6*S,8*S)-6-(5-(2-Aminopyrimidin-5-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

To a solution of 2-(2-aminopyrimidin-5-yl)-2-oxoethyl (6R,8R)-2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate (0.2 g, 0.39 mmol, 1 equiv) and ammonium acetate (0.6 g, 7.78 mol, 20 equiv) in toluene (5 mL) was added AcOH (0.5 ml). The reaction mixture was stirred 2 h at 90° C., then cooled to room temperature and quenched with water, extracted with ethyl acetate, washed with brine, dried over Na₂SO₄ and concentrated under vacuum. The residue was purified by silica gel chromatography with EA/PE (0-66%) to yield 6-(5-(2-aminopyrimidin-5-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one as yellow oil, which was further purified by chiral HPLC to yield (6S*,8S*)-6-(5-(2-aminopyrimidin-5-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one.

LC/MS: mass calculated for C₂₂H₁₇ClFN₁₁O: 505.13, measured (ES, m/z): 506.05 [M+H]⁺. ¹H NMR (400 MHz, Methanol-d₄) δ 9.47 (s, 1H), 8.55-8.65 (m, 2H), 7.92 (dd, J=8.7, 7.5 Hz, 1H), 7.63 (dd, J=8.7, 1.6 Hz, 1H), 7.40 (s, 1H), 6.58 (d, J=2.3 Hz, 1H), 5.56 (t, J=8.1 Hz, 1H), 3.09-3.21 (m, 1H), 2.81-2.93 (m, 1H), 2.04-2.16 (m, 1H), 1.27 (d, J=7.0 Hz, 3H). ¹⁹F NMR (376 MHz, Methanol-d₄) δ −114.48.

### Example 120: N-(5-(2-((6*R,8*R)-2-(3-Chloro-2-fluoro-6-(1H-tetrazol-1-yl)phenyl)-8-methyl-4-oxo-4,6,7,8-tetrahydropyrrolo[1,2-a]pyrimidin-6-yl)-1H-imidazol-5-yl)pyridin-2-yl)cyclopropanecarboxamide

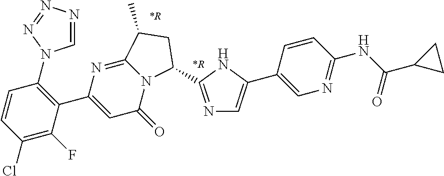 [SMILES: C[C@@H]1C[C@H](c2ncc(-c3ccc(NC(=O)C4CC4)nc3)[nH]2)n2c1nc(-c1c(-n3cnnn3)ccc(Cl)c1F)cc2=O]

LC/MS: mass calculated for C₂₇H₂₂ClFN₁₀O₂: 572.16, measured (ES, m/z): 573.30 [M+H]⁺. ¹H NMR (300 MHz, Methanol-d₄) δ 9.49 (s, 1H), 8.64 (s, 1H), 8.00-8.15 (m, 2H), 7.92 (dd, J=8.7, 7.5 Hz, 1H), 7.63 (dd, J=8.7, 1.6 Hz, 1H), 7.47 (s, 1H), 6.65 (dd, J=2.4, 0.8 Hz, 1H), 5.75 (dd, J=9.2, 1.8 Hz, 1H), 3.56-3.62 (m, 1H), 2.60-2.70 (m, 1H), 2.19-2.36 (m, 1H), 1.83-1.95 (m, 1H), 1.12 (d, J=7.0 Hz, 3H), 0.96-1.06 (m, 2H), 0.85-0.95 (m, 2H). ¹⁹F NMR (282 MHz, Methanol-d₄) δ −114.57.

### Example 121: N-(5-(2-((6*R,8*S)-2-(3-Chloro-2-fluoro-6-(1H-tetrazol-1-yl)phenyl)-8-methyl-4-oxo-4,6,7,8-tetrahydropyrrolo[1,2-a]pyrimidin-6-yl)-1H-imidazol-5-yl)pyridin-2-yl)cyclopropanecarboxamide

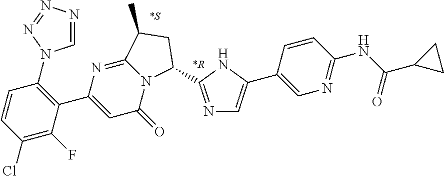 [SMILES: C[C@H]1C[C@H](c2ncc(-c3ccc(NC(=O)C4CC4)nc3)[nH]2)n2c1nc(-c1c(-n3cnnn3)ccc(Cl)c1F)cc2=O]

LC/MS: mass calculated for C₂₇H₂₂ClFN₁₀O₂: 572.16, measured (ES, m/z): 573.30 [M+H]⁺. ¹H NMR (300 MHz, Methanol-d₄) δ 9.49 (s, 1H), 8.64 (s, 1H), 8.00-8.15 (m, 2H), 7.92 (dd, J=8.7, 7.5 Hz, 1H), 7.63 (dd, J=8.7, 1.6 Hz, 1H), 7.48 (s, 1H), 6.65 (dd, J=2.4, 0.8 Hz, 1H), 5.75 (dd, J=9.1, 1.8 Hz, 1H), 3.42-3.72 (m, 1H), 2.61-2.67 (m, 1H), 2.19-2.36 (m, 1H), 1.83-1.95 (m, 1H), 1.12 (d, J=7.0 Hz, 3H), 0.96-1.06 (m, 2H), 0.87-0.96 (m, 2H). ¹⁹F NMR (282 MHz, Methanol-d₄) δ −114.58.

### Example 122: N-(5-(2-((6*S,8*R)-2-(3-Chloro-2-fluoro-6-(1H-tetrazol-1-yl)phenyl)-8-methyl-4-oxo-4,6,7,8-tetrahydropyrrolo[1,2-a]pyrimidin-6-yl)-1H-imidazol-5-yl)pyridin-2-yl)cyclopropanecarboxamide

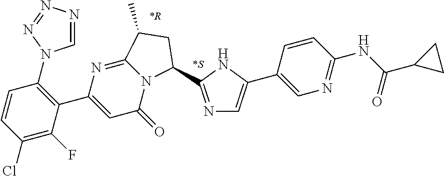 [SMILES: C[C@@H]1C[C@@H](c2ncc(-c3ccc(NC(=O)C4CC4)nc3)[nH]2)n2c1nc(-c1c(-n3cnnn3)ccc(Cl)c1F)cc2=O]

LC/MS: mass calculated for C₂₇H₂₂ClFN₁₀O₂: 572.16, measured (ES, m/z): 573.30 [M+H]⁺. ¹H NMR (300 MHz, Methanol-d₄) δ 9.47 (s, 1H), 8.67 (s, 1H), 8.02-8.15 (m, 2H), 7.92 (dd, J=8.7, 7.5 Hz, 1H), 7.63 (dd, J=8.6, 1.6 Hz, 1H), 7.50 (s, 1H), 6.55-6.62 (m, 1H), 5.57 (t, J=8.1 Hz, 1H), 3.21-3.10 (m, 1H), 2.80-2.96 (m, 1H), 2.07-2.20 (m, 1H), 1.86-1.92 (m, 1H), 1.29 (d, J=7.4 Hz, 3H), 0.97-1.07 (m, 2H), 0.86-0.96 (m, 2H). ¹⁹F NMR (282 MHz, Methanol-d₄) δ −114.47.

### Example 123: N-(5-(2-((6*S,8*S)-2-(3-Chloro-2-fluoro-6-(1H-tetrazol-1-yl)phenyl)-8-methyl-4-oxo-4,6,7,8-tetrahydropyrrolo[1,2-a]pyrimidin-6-yl)-1H-imidazol-5-yl)pyridin-2-yl)cyclopropanecarboxamide

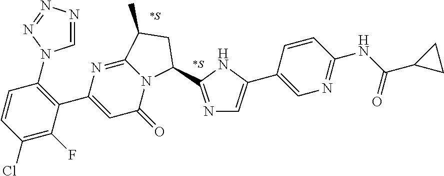 [SMILES: C[C@H]1C[C@@H](c2ncc(-c3ccc(NC(=O)C4CC4)nc3)[nH]2)n2c1nc(-c1c(-n3cnnn3)ccc(Cl)c1F)cc2=O]

LC/MS: mass calculated for C₂₇H₂₂ClFN₁₀O₂: 572.16, measured (ES, m/z): 573.30 [M+H]⁺. ¹H NMR (300 MHz, Methanol-d₄) δ 9.47 (s, 1H), 8.67 (s, 1H), 8.02-8.15 (m, 2H), 7.92 (dd, J=8.7, 7.5 Hz, 1H), 7.63 (dd, J=8.7, 1.7 Hz, 1H), 7.50 (s, 1H), 6.58 (dd, J=2.4, 0.7 Hz, 1H), 5.57 (t, J=8.1 Hz, 1H), 3.10-3.26 (m, 1H), 2.80-2.96 (m, 1H), 2.04-2.20 (m, 1H), 1.83-1.98 (m, 1H), 1.29 (d, J=7.1 Hz, 3H), 0.97-1.05 (m, 2H), 0.87-0.96 (m, 2H). ¹⁹F NMR (282 MHz, Methanol-d₄) δ −114.46.

### Example 124: Methyl (4-(2-((6*R,8*S)-2-(3-Chloro-2-fluoro-6-(1H-tetrazol-1-yl)phenyl)-8-methyl-4-oxo-4,6,7,8-tetrahydropyrrolo[1,2-a]pyrimidin-6-yl)-1H-imidazol-5-yl)phenyl)carbamate

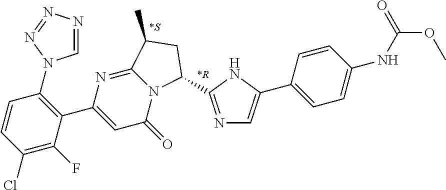 [SMILES: COC(=O)Nc1ccc(-c2cnc([C@H]3C[C@H](C)c4nc(-c5c(-n6cnnn6)ccc(Cl)c5F)cc(=O)n43)[nH]2)cc1]

LC/MS: mass calculated for C₂₆H₂₁ClFN₉O₃: 561.14, measured (ES, m/z): 562.25 [M+H]⁺. ¹H NMR (400 MHz, DMSO-d₆) δ 9.66 (s, 1H), 8.05-7.96 (m, 1H), 7.70 (dd, J=8.7, 1.5 Hz, 1H), 7.54-7.65 (m, 2H), 7.35-7.50 (m, 3H), 6.53 (d, J=2.0 Hz, 1H), 5.51 (dd, J=8.3, 6.8 Hz, 1H), 3.65 (s, 3H), 3.02-3.12 (m, 1H), 2.73-2.81 (m, 1H), 1.85-1.91 (m, 1H), 1.12 (d, J=7.1 Hz, 3H). ¹⁹F NMR (376 MHz, DMSO-d₆) δ −113.65.

### Example 125 Methyl (4-(2-((6*S,8*S)-2-(3-Chloro-2-fluoro-6-(1H-tetrazol-1-yl)phenyl)-8-methyl-4-oxo-4,6,7,8-tetrahydropyrrolo[1,2-a]pyrimidin-6-yl)-1H-imidazol-5-yl)phenyl)carbamate

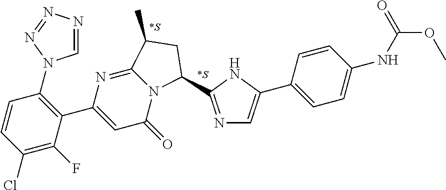 [SMILES: COC(=O)Nc1ccc(-c2cnc([C@@H]3C[C@H](C)c4nc(-c5c(-n6cnnn6)ccc(Cl)c5F)cc(=O)n43)[nH]2)cc1]

LC/MS: mass calculated for C₂₆H₂₁ClFN₉O₃: 561.14, measured (ES, m/z): 562.25[M+H]⁺. ¹H NMR (400 MHz, DMSO-d₆) δ 9.69 (s, 1H), 8.00 (dd, J=8.6, 7.6 Hz, 1H), 7.70 (dd, J=8.7, 1.5 Hz, 1H), 7.54-7.61 (m, 2H), 7.38-7.45 (m, 3H), 6.60 (d, J=2.1 Hz, 1H), 5.62 (dd, J=9.0, 1.6 Hz, 1H), 3.64 (s, 3H), 3.38-3.49 (m, 1H), 2.35-2.45 (m, 1H), 2.12-2.17 (m, 1H), 0.95 (d, J=7.0 Hz, 3H). ¹⁹F NMR (376 MHz, DMSO-d₆) δ −113.69.

### Example 126: Methyl (4-(2-((6*S,8*R)-2-(3-Chloro-2-fluoro-6-(1H-tetrazol-1-yl)phenyl)-8-methyl-4-oxo-4,6,7,8-tetrahydropyrrolo[1,2-a]pyrimidin-6-yl)-1H-imidazol-5-yl)phenyl)carbamate

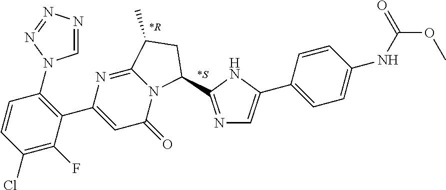 [SMILES: COC(=O)Nc1ccc(-c2cnc([C@@H]3C[C@@H](C)c4nc(-c5c(-n6cnnn6)ccc(Cl)c5F)cc(=O)n43)[nH]2)cc1]

### Step 1: 2-(4-((methoxycarbonyl)amino)phenyl)-2-oxoethyl 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate

A mixture of 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylic acid (300 mg, 0.768 mmol, 1.0 equiv) and potassium carbonate (106 mg, 0.77 mmol, 1.0 equiv) in DMF (4 mL) was stirred at room temperature for 30 mins. To which was added methyl 4-(2-chloroacetyl)phenylcarbamate (210 mg, 0.92 mmol, 1.2 eq.). The reaction was stirred at 20° C. for 1 h. Water was added, the mixture was extracted with ethyl acetate. The combined extracts were washed with water, saturated brine, and dried over anhydrous Na₂SO₄ and concentrated. The residue was purified by flash column chromatography on silica gel (MeOH/DCM, 0-5%) to yield 2-(4-(methoxycarbonylamino)phenyl)-2-oxoethyl 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate as a yellow solid. LC/MS: mass calculated, for C₂₆H₂₁ClFN₇O₆: 581.94, measured: 582.05 [M+H]⁺.

### Step 2: Methyl (4-(2-((6*S,8*R)-2-(3-Chloro-2-fluoro-6-(1H-tetrazol-1-yl)phenyl)-8-methyl-4-oxo-4,6,7,8-tetrahydropyrrolo[1,2-a]pyrimidin-6-yl)-1H-imidazol-5-yl)phenyl)carbamate

To a mixture of 2-(4-(methoxycarbonylamino)phenyl)-2-oxoethyl 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate (200 mg, 0.34 mmol, 1.0 equiv) and ammonium acetate (133 mg, 1.72 mmol, 5.0 equiv) in toluene (5 mL) was added acetic acid (10.4 mg, 0.172 mmol, 0.5 equiv). The reaction mixture was stirred at 90° C. for 1.5 h, concentrated and the residue was purified by reverse phase chromatography on C18 (80 g, CH₃CN/H₂O (0.05% CF₃COOH): 0>>>60%) to yield methyl 4-(2-(2-(3-chloro-2-fluoro-6-(1H-tetrazol-1-yl)phenyl)-8-methyl-4-oxo-4,6,7,8-tetrahydropyrrolo[1,2-a]pyrimidin-6-yl)-1H-imidazol-5-yl)phenylcarbamate as a yellow solid. LC/MS: mass calculated for C₂₆H₂₁ClFN₉O₃: 561.955, measured: 562.25 [M+H]⁺.

The racemic mixture was separated by prep-HPLC and prep-chiral-HPLC separation. The collected fractions were combined and concentrated under vacuum to yield 4-(2-((6R*,8S*)-2-(3-chloro-2-fluoro-6-(1H-tetrazol-1-yl)phenyl)-8-methyl-4-oxo-4,6,7,8-tetrahydropyrrolo[1,2-a]pyrimidin-6-yl)-1H-imidazol-5-yl)phenylcarbamate as a yellow solid; methyl 4-(2-((6S*,8R*)-2-(3-chloro-2-fluoro-6-(1H-tetrazol-1-yl)phenyl)-8-methyl-4-oxo-4,6,7,8-tetrahydropyrrolo[1,2-a]pyrimidin-6-yl)-1H-imidazol-5-yl)phenylcarbamate as an off-white solid; methyl 4-(2-((6R*,8R*)-2-(3-chloro-2-fluoro-6-(1H-tetrazol-1-yl)phenyl)-8-methyl-4-oxo-4,6,7,8-tetrahydropyrrolo[1,2-a]pyrimidin-6-yl)-1H-imidazol-5-yl)phenylcarbamate as an off-white solid; and methyl 4-(2-((6S*,8S*)-2-(3-chloro-2-fluoro-6-(1H-tetrazol-1-yl)phenyl)-8-methyl-4-oxo-4,6,7,8-tetrahydropyrrolo[1,2-a]pyrimidin-6-yl)-1H-imidazol-5-yl)phenylcarbamate as an off-white solid.

LC/MS: mass calculated for C₂₆H₂₁ClFN₉O₃: 561.14, measured (ES, m/z): 562.25 [M+H]⁺. ¹H NMR (400 MHz, DMSO-d₆) δ 9.66 (s, 1H), 9.63 (s, 1H), 7.95-8.04 (m, 1H), 7.70 (dd, J=8.7, 1.5 Hz, 1H), 7.58-5.64 (m, 2H), 7.40-7.47 (m, 3H), 6.52 (d, J=2.0 Hz, 1H), 5.50 (dd, J=8.4, 6.6 Hz, 1H), 3.65 (s, 3H), 3.02-3.12 (m, 1H), 2.72-2.80 (m, Hz, 1H), 1.84-1.91 (m, 1H), 1.11 (d, J=7.1 Hz, 3H). ¹⁹F NMR (376 MHz, DMSO-d₆) δ −113.67.

### Example 127: Methyl (4-(2-((6*R,8*R)-2-(3-Chloro-2-fluoro-6-(1H-tetrazol-1-yl)phenyl)-8-methyl-4-oxo-4,6,7,8-tetrahydropyrrolo[1,2-a]pyrimidin-6-yl)-1H-imidazol-5-yl)phenyl)carbamate

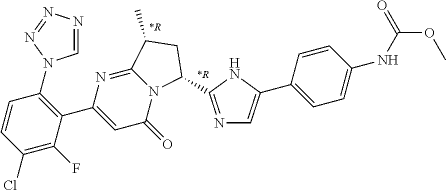 [SMILES: COC(=O)Nc1ccc(-c2cnc([C@H]3C[C@@H](C)c4nc(-c5c(-n6cnnn6)ccc(Cl)c5F)cc(=O)n43)[nH]2)cc1]

LC/MS: mass calculated for C₂₆H₂₁ClFN₉O₃: 561.14, measured (ES, m/z): 562.25 [M+H]⁺. ¹H NMR (400 MHz, DMSO-d₆) δ 9.70 (s, 1H), 7.98-8.06 (m, 1H), 7.72 (dd, J=8.6, 1.5 Hz, 1H), 7.56-7.65 (m, 2H), 7.41-7.51 (m, 3H), 6.62 (d, J=2.1 Hz, 1H), 5.65 (d, J=8.7 Hz, 1H), 3.65 (s, 3H), 3.47-3.41 (m, 1H), 2.40-2.45 (m, 1H), 2.14-2.22 (m, 1H), 0.96 (d, J=7.0 Hz, 3H). ¹⁹F NMR (376 MHz, DMSO-d₆) δ −113.66.

### Example 128: (6*R,8*R)-6-(5-(6-Amino-5-fluoro-2-methyl pyridin-3-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

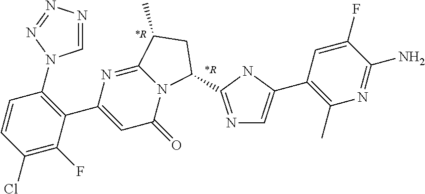 [SMILES: Cc1nc(N)c(F)cc1-c1cnc([C@H]2C[C@@H](C)c3nc(-c4c(-n5cnnn5)ccc(Cl)c4F)cc(=O)n32)[nH]1]

LC/MS: mass calculated for C₂₄H₁₉ClF₂N₁₀O: 536.14, measured (ES, m/z): 537.10 [M+H]⁺. ¹H NMR (400 MHz, DMSO-d₆) δ 12.25-12.35 (m, 1H), 9.76 (s, 1H), 8.06 (t, J=8.2 Hz, 1H), 7.77 (dd, J=8.7, 1.5 Hz, 1H), 7.58 (d, J=12.3 Hz, 1H), 7.19 (d, J=2.1 Hz, 1H), 6.63 (d, J=2.0 Hz, 1H), 6.01 (s, 2H), 5.61 (d, J=8.7 Hz, 1H), 3.42-3.55 (m, 1H), 2.33-2.46 (m, 4H), 2.10-2.23 (m, 1H), 0.98 (d, J=7.0 Hz, 3H). ¹⁹F NMR (376 MHz, DMSO-d₆) δ−113.72, 144.17.

### Example 129: (6*R,8*S)-6-(5-(6-Amino-5-fluoro-2-methyl pyridin-3-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

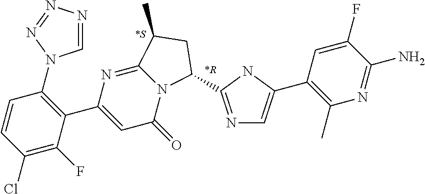 [SMILES: Cc1nc(N)c(F)cc1-c1cnc([C@H]2C[C@H](C)c3nc(-c4c(-n5cnnn5)ccc(Cl)c4F)cc(=O)n32)[nH]1]

LC/MS: mass calculated for C₂₄H₁₉ClF₂N₁₀O: 536.14, measured (ES, m/z): 537.10 [M+H]⁺. ¹H NMR (400 MHz, Methanol-d₄) δ 9.49 (s, 1H), 7.88-7.96 (m, 2H), 7.63 (dd, J=8.7, 1.6 Hz, 1H), 7.43 (s, 1H), 6.60 (d, J=2.2 Hz, 1H), 5.67 (t, J=8.0 Hz, 1H), 3.18-3.28 (m, 1H), 2.88-3.00 (m, 1H), 2.56 (s, 3H), 2.00-2.13 (m, 1H), 1.26 (d, J=7.2 Hz, 3H). ¹⁹F NMR (376 MHz, Methanol-d₄) δ −76.99, −114.53, 141.68.

### Example 130: (6*S,8*S)-6-(5-(6-Amino-5-fluoro-2-methyl pyridin-3-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

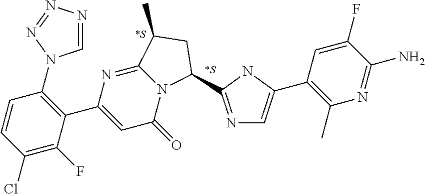 [SMILES: Cc1nc(N)c(F)cc1-c1cnc([C@@H]2C[C@H](C)c3nc(-c4c(-n5cnnn5)ccc(Cl)c4F)cc(=O)n32)[nH]1]

LC/MS: mass calculated for C₂₄H₁₉ClF₂N₁₀O: 536.14, measured (ES, m/z): 537.20 [M+H]⁺. ¹H NMR (400 MHz, DMSO-d₆) δ 9.65 (s, 1H), 7.88-8.02 (m, 2H), 7.65-7.72 (m, 1H), 7.51 (s, 1H), 6.63 (d, J=2.0 Hz, 1H), 5.72 (dd, J=9.1, 2.2 Hz, 1H), 3.32-3.42 (m, 1H), 2.40-2.49 (m, 4H), 2.15-2.28 (m, 1H), 0.96 (d, J=6.9 Hz, 3H). ¹⁹F NMR (376 MHz, DMSO-d₆) δ −74.31, 113.73.

### Example 131: (S)-6′-(5-(2-Amino-4-methylthiazol-5-yl)-1H-imidazol-2-yl)-2′-(3-chloro-2-fluoro-6-(1H-tetrazol-1-yl)phenyl)-6′,7′-dihydro-4′H-spiro[cyclopropane-1,8′-pyrrolo[1,2-a]pyrimidin]-4′-one

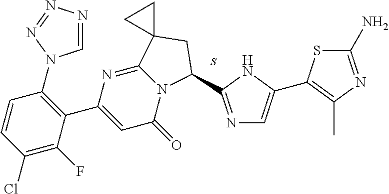

LC/MS: mass calculated for C₂₃H₁₈ClFN₁₀OS: 536.11, measured (ES, m/z): 537.15 [M+H]⁺. ¹H NMR (400 MHz, DMSO-d₆) δ 12.27 (s, 1H), 9.72 (s, 1H), 8.00-8.08 (m, 1H), 7.73 (dd, J=8.6, 1.4 Hz, 1H), 7.07 (s, 2H), 6.53 (d, J=2.3 Hz, 1H), 5.66 (dd, J=9.5, 3.3 Hz, 1H), 2.74 (dd, J=13.0, 9.5 Hz, 1H), 2.14-2.25 (m, 4H), 0.97-1.09 (m, 2H), 0.70-0.88 (m, 2H). ¹⁹F NMR (376 MHz, DMSO-d₆) δ −113.46.

### Example 132: (R)-6′-(5-(2-Amino-4-methylthiazol-5-yl)-1H-imidazol-2-yl)-2′-(3-chloro-2-fluoro-6-(1H-tetrazol-1-yl)phenyl)-6′,7′-dihydro-4′H-spiro[cyclopropane-1,8′-pyrrolo[1,2-a]pyrimidin]-4′-one

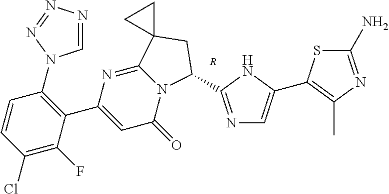

LC/MS: mass calculated for C₂₃H₁₈ClFN₁₀OS: 536.11, measured (ES, m/z): 537.15 [M+H]⁺. ¹H NMR (400 MHz, Methanol-d₄) δ 9.44 (s, 1H), 7.91 (dd, J=8.7, 7.5 Hz, 1H), 7.60 (dd, J=8.6, 1.6 Hz, 1H), 7.31 (s, 1H), 6.55 (d, J=2.5 Hz, 1H), 5.79 (dd, J=9.5, 4.0 Hz, 1H), 2.82 (dd, J=13.1, 9.4 Hz, 1H), 2.32-2.43 (m, 4H), 0.87-1.19 (m, 4H). ¹⁹F NMR (376 MHz, Methanol-d₄) δ−114.50.

### Example 133: (R)-6′-(5-(6-Amino-2-fluoropyridin-3-yl)-1H-imidazol-2-yl)-2′-(3-chloro-2-fluoro-6-(1H-tetrazol-1-yl)phenyl)-6′,7′-dihydro-4′H-spiro[cyclopropane-1,8′-pyrrolo[1,2-a]pyrimidin]-4′-one

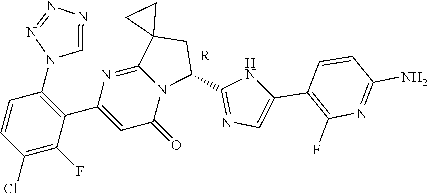

LC/MS: mass calculated for C₂₄H₁₇ClF₂N₁₀O: 534.12, measured (ES, m/z): 535.30 [M+H]⁺. ¹H NMR (400 MHz, DMSO-d₆) δ 12.20 (s, 1H), 9.72 (s, 1H), 8.00-8.08 (m, 2H), 7.74 (dd, J=8.7, 1.4 Hz, 1H), 7.12 (s, 1H), 6.53 (d, J=2.2 Hz, 1H), 6.39 (dd, J=8.3, 2.1 Hz, 1H), 6.26 (s, 2H), 5.65-5.75 (m, 1H), 2.76 (dd, J=12.9, 9.5 Hz, 1H), 2.15-2.29 (m, 1H), 0.96-1.11 (m, 2H), 0.72-0.87 (m, 2H). ¹⁹F NMR (376 MHz, DMSO-d₆) δ −70.78, −113.61.

### Example 134: (S)-6′-(5-(6-Amino-2-fluoropyridin-3-yl)-1H-imidazol-2-yl)-2′-(3-chloro-2-fluoro-6-(1H-tetrazol-1-yl)phenyl)-6′,7′-dihydro-4′H-spiro[cyclopropane-1,8′-pyrrolo[1,2-a]pyrimidin]-4′-one

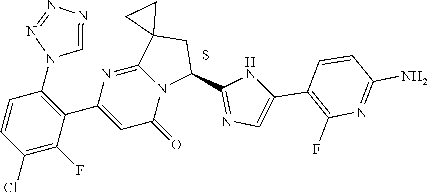

### Step 1: Methyl (S)-4-oxo-5-azaspiro[2.4]heptane-6-carboxylate

To a solution of 5-tert-butyl 6-methyl 4-oxo-5-azaspiro[2.4]heptane-5,6-dicarboxylate (4.0 g, 14.85 mmol, 1.0 equiv.) in dichloromethane (50 mL) was added TFA (15 mL). The resulting mixture was stirred at room temperature for 1 h and the solvent was removed. The residue was dissolved in DCM and washed with aqueous NaHCO₃. The combined extracts were washed with water, saturated brine and dried over anhydrous Na₂SO₄, and concentrated to yield methyl (S)-4-oxo-5-azaspiro[2.4]heptane-6-carboxylate as a white solid.

### Step 2: Methyl (S)-4-methoxy-5-azaspiro[2.4]hept-4-ene-6-carboxylate

To a solution of methyl (S)-4-oxo-5-azaspiro[2.4]heptane-6-carboxylate (2.5 g, 14.78 mmol, 1.0 equiv.) in DCM (50 mL) was added trimethyloxonium tetrafluoroborate (3.3 g, 22.17 mmol, 1.5 equiv.). The resulting mixture was stirred at room temperature. for 3 h. The reaction was quenched with saturated aqueous solution of sodium hydrogen carbonate. The resulting mixture was extracted with CH₂Cl₂. The organic layers were combined, dried, and concentrated under vacuum to yield methyl (S)-4-methoxy-5-azaspiro[2.4]hept-4-ene-6-carboxylate as a yellow oil.

### Step 3: Methyl (S)-4-amino-5-azaspiro[2.4]hept-4-ene-6-carboxylate

To a solution of methyl (S)-4-methoxy-5-azaspiro[2.4]hept-4-ene-6-carboxylate (2.2 g, 12.01 mmol 1.0 equiv.) in methanol (25 mL) was added ammonium chloride (771 mg, 14.41 mmol, 1.2 equiv.). The resulting mixture was stirred at 80° C. for 2 h, cooled to room temperature and filtered through a pad of CELITE. The filtrate was concentrated to yield methyl (S)-4-amino-5-azaspiro[2.4]hept-4-ene-6-carboxylate as an off-white solid.

### Step 4: Methyl (S)-2′-hydroxy-4′-oxo-6′,7′-dihydro-4′H-spiro[cyclopropane-1,8′-pyrrolo[1,2-a]pyrimidine]-6′-carboxylate

To a mixture of methyl (S)-4-amino-5-azaspiro[2.4]hept-4-ene-6-carboxylate (2.0 g, 11.89 mmol, 1.0 equiv.) in DMF (25 mL) with N-ethyl-N-isopropylpropan-2-amine (6.3 mL, 35.67 mmol, 3.0 equiv.) and EDCl (4.6 g, 23.78 mmol, 2.0 equiv.) was added ethyl potassium malonate (2.6 g, 15.46 mmol, 1.3 equiv.). The reaction mixture was stirred at 60° C. for 16h, concentrated and the residue was purified by reverse phase chromatography on C18 (330 g, CH₃CN/H₂O (0.05% CF₃COOH): 0-50%) to yield methyl (S)-2′-hydroxy-4′-oxo-6′,7′-dihydro-4′H-spiro[cyclopropane-1,8′-pyrrolo[1,2-a]pyrimidine]-6′-carboxylate as a white solid.

### Step 5: Methyl (S)-4′-oxo-2′-(((trifluoromethyl)sulfonyl)oxy)-6′,7′-dihydro-4′H-spiro[cyclopropane-1,8′-pyrrolo[1,2-a]pyrimidine]-6′-carboxylate

To a solution of methyl (S)-2′-hydroxy-4′-oxo-6′,7′-dihydro-4′H-spiro[cyclopropane-1,8′-pyrrolo[1,2-a]pyrimidine]-6′-carboxylate (1.5 g, 6.35 mmol, 1.0 equiv.) in DMF (20 mL) was added cesium carbonate (3.1 g, 9.53 mmol, 1.5 equiv.), followed by addition of trifluoro-N-phenyl-N-(trifluoromethylsulfonyl)methane sulfonamide (2.5 g, 6.99 mmol, 1.1 equiv.). The reaction mixture was stirred for 1 h at room temperature, then quenched with water, extracted with EA, washed with brine, dried over Na₂SO₄ and concentrated under vacuum. The residue was purified by silica gel chromatography with EA/PE (0-30%) to methyl (S)-4′-oxo-2′-(((trifluoromethyl)sulfonyl)oxy)-6′,7′-dihydro-4′H-spiro[cyclopropane-1,8′-pyrrolo[1,2-a]pyrimidine]-6′-carboxylate as light yellow oil. LC/MS: mass calculated, for C₁₂H₁₁F₃N₂O₆S: 368.03, measured: 369.05 [M+H]⁺.

### Step 6: Methyl (S)-2′-(6-amino-3-chloro-2-fluorophenyl)-4′-oxo-6′,7′-dihydro-4′H-spiro[cyclopropane-1,8′-pyrrolo[1,2-a]pyrimidine]-6′-carboxylate

To a solution of methyl (S)-4′-oxo-2′-(((trifluoromethyl)sulfonyl)oxy)-6′,7′-dihydro-4′H-spiro[cyclopropane-1,8′-pyrrolo[1,2-a]pyrimidine]-6′-carboxylate (1.3 g, 3.39 mmol, 1.0 equiv.) and 6-amino-3-chloro-2-fluorophenylboronic acid (1.3 g, 6.79 mmol, 2.0 equiv.) in 1,4-dioxane (20 mL) were added cesium fluoride (1.5 g, 10.18 mmol, 3.0 equiv.) and Pd(PPh₃)₄ (0.39 g, 0.34 mmol, 0.1 equiv.) under N₂. The reaction mixture was stirred for 2 h at 90° C., then cooled to room temperature. and quenched with water, extracted with ethyl acetate, washed with brine, dried over Na₂SO₄ and concentrated under vacuum. The residue was purified by silica gel chromatography with EA/PE (0-100%) to yield methyl (S)-2′-(6-amino-3-chloro-2-fluorophenyl)-4′-oxo-6′,7′-dihydro-4′H-spiro[cyclopropane-1,8′-pyrrolo[1,2-a]pyrimidine]-6′-carboxylate as a light yellow solid. LC/MS: mass calculated, for C₁₇H₁₁ClFN₃O₃: 363.08, measured: 364.0 [M+H]⁺.

### Step 7: (S)-2′-(6-amino-3-chloro-2-fluorophenyl)-4′-oxo-6′,7′-dihydro-4′H-spiro[cyclopropane-1,8′-pyrrolo[1,2-a]pyrimidine]-6′-carboxylic acid

To a solution of methyl (S)-2′-(6-amino-3-chloro-2-fluorophenyl)-4′-oxo-6′,7′-dihydro-4′H-spiro[cyclopropane-1,8′-pyrrolo[1,2-a]pyrimidine]-6′-carboxylate (1.1 g, 3.02 mmol, 1.0 equiv.) in THE (10 mL), MeOH (5 mL) and water (5 mL) was added lithiumhydroxide (0.22 g, 9.07 mmol, 3.0 equiv.). The reaction mixture was stirred for 2 h at room temperature. The pH was adjusted to 5˜6 with 2M HCl and the resulting mixture was extracted with ethyl acetate. The organic layers were combined, dried over Na₂SO₄, filtered and concentrated to yield (S)-2′-(6-amino-3-chloro-2-fluorophenyl)-4′-oxo-6′,7′-dihydro-4′H-spiro[cyclopropane-1,8′-pyrrolo[1,2-a]pyrimidine]-6′-carboxylic acid as a light brown solid. LC/MS: mass calculated, for C₁₆H₁₃ClFN₃O₃: 349.06, measured: 350.10 [M+H]⁺.

### Step 8: (S)-2′-(3-chloro-2-fluoro-6-(1H-tetrazol-1-yl)phenyl)-4′-oxo-6′,7′-dihydro-4′H-spiro[cyclopropane-1,8′-pyrrolo[1,2-a]pyrimidine]-6′-carboxylic acid

To a solution of (S)-2′-(6-amino-3-chloro-2-fluorophenyl)-4′-oxo-6′,7′-dihydro-4′H-spiro[cyclopropane-1,8′-pyrrolo[1,2-a]pyrimidine]-6′-carboxylic acid (0.50 g, 1.43 mmol, 1.0 equiv.) in acetic acid (10 mL) were added trimethoxymethane (1.5 g, 14.30 mmol, 10.0 equiv.), and TMSN₃ (1.6 g, 14.30 mmol, 10.0 equiv.). The resulting mixture was stirred overnight at room temperature, then concentrated under vacuum. The residue was purified by reverse column chromatography with CH₃CN/water (5%-50%) to yield (S)-2′-(3-chloro-2-fluoro-6-(1H-tetrazol-1-yl)phenyl)-4′-oxo-6′,7′-dihydro-4′H-spiro[cyclopropane-1,8′-pyrrolo[1,2-a]pyrimidine]-6′-carboxylic acid as a light yellow solid. LC/MS: mass calculated, for C₁₇H₁₂ClFN₆O₃: 402.06, measured: 403.05 [M+H]⁺.

### Step 9: 2-(6-Amino-2-fluoropyridin-3-yl)-2-oxoethyl 2′-(3-chloro-2-fluoro-6-(1H-tetrazol-1-yl)phenyl)-4′-oxo-6′,7′-dihydro-4′H-spiro[cyclopropane-1,8′-pyrrolo[1,2-a]pyrimidine]-6′-carboxylate

To a solution of 2′-(3-chloro-2-fluoro-6-(1H-tetrazol-1-yl)phenyl)-4′-oxo-6′,7′-dihydro-4′H-spiro[cyclopropane-1,8′-pyrrolo[1,2-a]pyrimidine]-6′-carboxylic acid (100 mg, 0.25 mmol, 1.0 equiv) in DMF (5 mL) was added potassium carbonate (103 mg, 0.74 mmol, 3.0 equiv). After the mixture was stirred for 30 min, 1-(6-amino-2-fluoropyridin-3-yl)-2-bromoethan-1-one (87 mg, 0.37 mmol, 1.5 equiv) was added. The reaction mixture was stirred for 2 h at room temperature, then concentrated and the residue was purified by reverse column chromatography with CH₃CN/0.05% TFA water (5%-80%) to yield 2-(6-amino-2-fluoropyridin-3-yl)-2-oxoethyl 2′-(3-chloro-2-fluoro-6-(1H-tetrazol-1-yl)phenyl)-4′-oxo-6′,7′-dihydro-4′H-spiro[cyclopropane-1,8′-pyrrolo[1,2-a]pyrimidine]-6′-carboxylate as a light yellow solid.

### Step 10: (*S)-6′-(5-(6-Amino-2-fluoropyridin-3-yl)-1H-imidazol-2-yl)-2′-(3-chloro-2-fluoro-6-(1H-tetrazol-1-yl)phenyl)-6′,7′-dihydro-4′H-spiro[cyclopropane-1,8′-pyrrolo[1,2-a]pyrimidin]-4′-one

To a solution of 2-(2-amino-4-methylthiazol-5-yl)-2-oxoethyl 2′-(3-chloro-2-fluoro-6-(1H-tetrazol-1-yl)phenyl)-4′-oxo-6′,7′-dihydro-4′H-spiro[cyclopropane-1,8′-pyrrolo[1,2-a]pyrimidine]-6′-carboxylate (90 mg, 0.16 mmol, 1.0 equiv) in toluene (15 mL) was added ammonium acetate (249 mg, 3.23 mmol, 2.0 equiv) followed by the addition of acetic acid (0.5 mL) under N₂. The reaction mixture was stirred for 3 h at 100° C., then cooled to room temperature and concentrated under vacuum. The residue was purified by silica gel chromatography with MeOH/DCM (0-10%) to yield a residue, which was further purified by Chiral-HPLC with Hex (0.1% DEA):EtOH=70:30 to yield (*S)-6′-(5-(6-amino-2-fluoropyridin-3-yl)-1H-imidazol-2-yl)-2′-(3-chloro-2-fluoro-6-(1H-tetrazol-1-yl)phenyl)-6′,7′-dihydro-4′H-spiro[cyclopropane-1,8′-pyrrolo[1,2-a]pyrimidin]-4′-one as a white solid LC/MS: mass calculated for C₂₄H₁₇ClF₂N₁₀O: 534.12, measured (ES, m/z): 535.25 [M+H]⁺. ¹H NMR (400 MHz, DMSO-d₆) δ 12.20 (s, 1H), 9.72 (s, 1H), 8.04 (t, J=8.2 Hz, 2H), 7.74 (dd, J=8.7, 1.4 Hz, 1H), 7.12 (s, 1H), 6.53 (d, J=2.2 Hz, 1H), 6.39 (dd, J=8.2, 2.1 Hz, 1H), 6.26 (s, 2H), 5.67-5.74 (m, 1H), 2.76 (dd, J=13.0, 9.5 Hz, 1H), 2.17-2.26 (m, 1H), 0.96-1.14 (m, 2H), 0.73-0.88 (m, 2H). ¹⁹F NMR (376 MHz, DMSO-d₆) δ −70.78, −113.62.

### Example 135: (6*R,8*R)-2-(3-Chloro-2-fluoro-6-(1H-tetrazol-1-yl)phenyl)-8-methyl-6-(5-(1-methyl-1H-1,2,3-triazol-5-yl)-1H-imidazol-2-yl)-7,8-dihydropyrrolo[1,2-a]pyrimidin-4(6H)-one

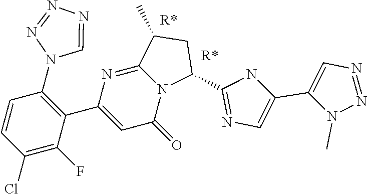

LC/MS: mass calculated for C₂₁H₁₇ClFN₁₁O: 493.13, measured (ES, m/z): 494.05 [M+H]⁺. ¹H NMR (400 MHz, DMSO-d₆) δ 12.71 (s, 1H), 9.77 (s, 1H), 8.07 (dd, J=8.6, 7.7 Hz, 1H), 7.84 (s, 1H), 7.77 (dd, J=8.7, 1.5 Hz, 1H), 7.63 (s, 1H), 6.64 (d, J=2.0 Hz, 1H), 5.61-5.68 (m, 1H), 4.13 (s, 3H), 3.40-3.48 (m, 1H), 2.37-2.46 (m, 1H), 2.14-2.26 (m, 1H), 1.25 (s, 1H), 1.00 (d, J=7.0 Hz, 3H). ¹⁹F NMR (376 MHz, DMSO-d₆) δ −113.86.

### Example 136: (6*S,8*S)-2-(3-Chloro-2-fluoro-6-(1H-tetrazol-1-yl)phenyl)-8-methyl-6-(5-(1-methyl-1H-1,2,3-triazol-5-yl)-1H-imidazol-2-yl)-7,8-dihydropyrrolo[1,2-a]pyrimidin-4(6H)-one

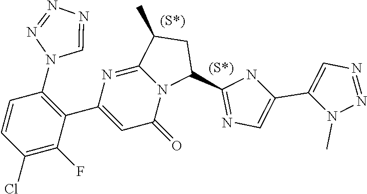

### Step 1: 2-(1-Methyl-1H-1,2,3-triazol-5-yl)-2-oxoethyl 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate

To a solution of 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylic acid (0.2 g, 0.51 mmol, 1 equiv) in DMF (10 mL) was added K₂CO₃ (0.21 g, 1.53 mmol, 3 equiv) under N₂. The reaction mixture was stirred for 0.5 h, then 2-bromo-1-(1-methyl-1H-1, 2, 3-triazol-5-yl) ethan-1-one (0.2 g, 1.02 mmol, 1.0 equiv) was added.

The reaction mixture was stirred 1 h at room temperature, then concentrated under vacuum. The residue was purified by reverse-phase chromatography (C18, 330 g, CH₃CN/H₂O (0.05% TFA)=10%-70%) to 2-(1-methyl-1H-1,2,3-triazol-5-yl)-2-oxoethyl 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate as a red oil. LC/MS: mass calculated for C₂₁H₁₇ClFN₉O₄: 513.11, measured: 514.00 [M+H]⁺.

### Step 2: (6*S,8*S)-2-(3-Chloro-2-fluoro-6-(1H-tetrazol-1-yl)phenyl)-8-methyl-6-(5-(1-methyl-1H-1,2,3-triazol-5-yl)-1H-imidazol-2-yl)-7,8-dihydropyrrolo[1,2-a]pyrimidin-4(6H)-one

A mixture of 2-(1-methyl-1H-1,2,3-triazol-5-yl)-2-oxoethyl 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate (0.2 g, 0.38 mmol, 1 equiv) and ammonium acetate (0.6 g, 7.78 mmol, 20 equiv) in toluene (5 mL) and AcOH (0.5 mL) was stirred for 2 h at 90° C., then cooled to room temperature and quenched with water, extracted with ethyl acetate, washed with brine, dried over Na₂SO₄ and concentrated under vacuum. The residue was purified by silica gel chromatography with EA/PE (0-66%) to yield 2-(3-chloro-2-fluoro-6-(1H-tetrazol-1-yl)phenyl)-8-methyl-6-(5-(1-methyl-1H-1,2,3-triazol-5-yl)-1H-imidazol-2-yl)-7,8-dihydropyrrolo[1,2-a]pyrimidin-4(6H)-one as yellow oil, which was further purified by chiral HPLC to yield (6*S,8*S)-2-(3-chloro-2-fluoro-6-(1H-tetrazol-1-yl)phenyl)-8-methyl-6-(5-(1-methyl-1H-1,2,3-triazol-5-yl)-1H-imidazol-2-yl)-7,8-dihydropyrrolo[1,2-a]pyrimidin-4(6H)-one.

LC/MS: mass calculated for C₂₁H₁₇ClFN₁₁O: 493.13, measured: 494.15 [M+H]⁺. ¹H NMR (400 MHz, Methanol-d₄) δ 9.54 (s, 1H), 8.15 (s, 1H), 8.04 (s, 1H), 7.90-7.98 (m, 1H), 7.60-765 (m, 1H), 6.67 (d, J=2.3 Hz, 1H), 5.70-5.80 (m, 1H), 4.28 (s, 3H), 3.21-3.32 (m, 1H), 2.99-3.08 (m, 1H), 1.97-2.13 (m, 1H), 1.28-1.32 (m, 1H), 1.22-1.27 (m, 3H).

### Example 137: (6S*,8R*)-6-(5-(6-Amino-2-fluoropyridin-3-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

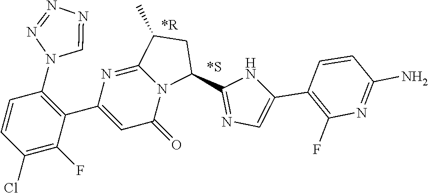

LC/MS: mass calculated for C₂₃H₁₇ClF₂N₁₀O: 522.12, measured: 523.20 [M+H]⁺. ¹H NMR (400 MHz, DMSO-d₆) δ 9.62 (s, 1H), 7.97 (t, J=8.2 Hz, 2H), 7.67 (dd, J=8.7, 1.4 Hz, 1H), 7.12 (s, 1H), 6.51 (d, J=2.0 Hz, 1H), 6.41 (dd, J=8.2, 2.1 Hz, 1H), 5.49 (dd, J=8.6, 6.0 Hz, 1H), 3.00-3.06 (m, 1H), 2.70-2.80 (m, 1H), 1.80-1.90 (m, 1H), 1.02-1.12 (m, 3H).

### Example 138: (6R*,8R*)-6-(5-(6-Amino-2-fluoropyridin-3-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

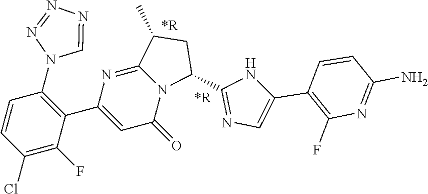

LC/MS: mass calculated for C₂₃H₁₇ClF₂N₁₀O: 522.12, measured: 545.10 [M+Na]⁺. ¹H NMR (400 MHz, DMSO-d₆) δ 9.67 (s, 1H), 7.85-8.08 (m, 2H), 7.69 (dd, J=8.7, 1.4 Hz, 1H), 7.11 (s, 1H), 6.58 (d, J=2.0 Hz, 1H), 6.38 (dd, J=8.3, 2.1 Hz, 1H), 5.60 (d, J=8.7 Hz, 1H), 3.41 (d, J=9.0 Hz, 1H), 2.30-2.40 (m, 1H), 2.07-2.19 (m, 1H), 0.90-1.00 (m, 3H).

### Example 139: (1R,3S)-3-(5-(6-Amino-2-fluoropyridin-3-yl)-1H-imidazol-2-yl)-7-(3-chloro-2-fluoro-6-(1H-tetrazol-1-yl)phenyl)-1-ethoxy-2,3-dihydroindolizin-5(1H)-one

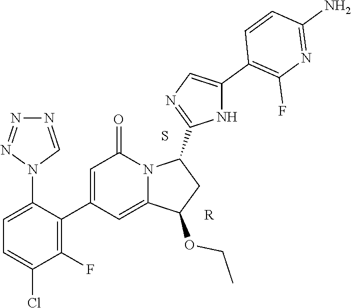

### Step 1: Methyl (S)-7-(6-((tert-butoxycarbonyl)amino)-3-chloro-2-fluorophenyl)-5-oxo-1,2,3,5-tetrahydroindolizine-3-carboxylate

A mixture of methyl (S)-7-(6-amino-3-chloro-2-fluorophenyl)-5-oxo-1,2,3,5-tetrahydroindolizine-3-carboxylate (2.0 g, 5.94 mmol, 1.0 equiv.) and (Boc)₂O (8.9 mL, 29.70 mmol, 5.0 equiv.) in toluene (20 mL) was heated at reflux overnight. The reaction solution was concentrated and the residue was purified by silica gel chromatography (0→60% EA/PE) to yield methyl (S)-7-(6-((tert-butoxycarbonyl)amino)-3-chloro-2-fluorophenyl)-5-oxo-1,2,3,5-tetrahydroindolizine-3-carboxylate as a yellow solid.

### Step 2: Methyl (3S)-7-(6-((tert-butoxycarbonyl)amino)-3-chloro-2-fluorophenyl)-1-hydroxy-5-oxo-1,2,3,5-tetrahydroindolizine-3-carboxylate

To a mixture of methyl 7-(6-((tert-butoxycarbonyl)amino)-3-chloro-2-fluorophenyl)-5-oxo-1,2,3,5-tetrahydroindolizine-3-carboxylate (1.0 g, 2.29 mmol, 1.0 equiv.) in 1,4-dioxane (50 mL) was added SeO₂ (2.0 g, 18.02 mmol, 8.0 equiv.). The mixture was stirred at 100° C. for 3 h under N₂, then concentrated under vacuum. The residue was purified by flash column chromatography on silica gel (0→50% EA/PE) to yield methyl 7-(6-((tert-butoxycarbonyl)amino)-3-chloro-2-fluorophenyl)-1-hydroxy-5-oxo-1,2,3,5-tetrahydroindolizine-3-carboxylate as a yellow solid. LC/MS: mass calculated for C₂₁H₂₂ClFN₂O₆: 452.12, measured: 453.20 [M+H]⁺.

### Step 3: Methyl (3S)-7-(6-((tert-butoxycarbonyl)amino)-3-chloro-2-fluorophenyl)-1-ethoxy-5-oxo-1,2,3,5-tetrahydroindolizine-3-carboxylate

To a solution of methyl 7-(6-(bis(tert-butoxycarbonyl)amino)-3-chloro-2-fluorophenyl)-1-hydroxy-5-oxo-1,2,3,5-tetrahydroindolizine-3-carboxylate (400 mg, 0.72 mmol, 1.0 equiv.) in CHCl₃ (20 mL) was added silver oxide (896 mg, 7.23 mmol, 10.0 equiv.) followed by iodoethane (1.13 g, 7.23 mmol, 10.0 equiv.). The mixture was stirred at 50° C. for 24 h, cooled to room temperature and filtered through a pad of CELITE. The filtrate was concentrated to yield methyl (3S)-7-(6-((tert-butoxycarbonyl)amino)-3-chloro-2-fluorophenyl)-1-ethoxy-5-oxo-1,2,3,5-tetrahydroindolizine-3-carboxylate as a yellow solid, which was used in the next step without further purification. LC/MS (ES, m/z): mass calculated for C₂₉H₃₆ClFN₂O₈: 594.21, measured: 595.30 [M+H]⁺.

### Step 4: Methyl (3S)-7-(6-amino-3-chloro-2-fluorophenyl)-1-ethoxy-5-oxo-1,2,3,5-tetrahydroindolizine-3-carboxylate

To a solution of methyl (3S)-7-(6-{bis[(tert-butoxy)carbonyl]amino}-3-chloro-2-fluorophenyl)-1-ethoxy-5-oxo-1,2,3,5-tetrahydroindolizine-3-carboxylate (250 mg) in dichloromethane (15 mL) was added trifluoro acetic acid (3 mL). The reaction mixture was stirred at room temperature for 1 h, then concentrated, and the residue was purified by reverse column chromatography on C18 column with CH₃CN/0.05% TFA water (5%→80%) to yield methyl (3S)-7-(6-amino-3-chloro-2-fluorophenyl)-1-ethoxy-5-oxo-1,2,3,5-tetrahydroindolizine-3-carboxylate as a light yellow solid. LC/MS (ES, m/z): mass calculated for C₁₉H₂₀ClFN₂O₄: 394.11, measured: 395.05 [M+H]+.

### Step 5: (3S)-7-(6-Amino-3-chloro-2-fluorophenyl)-1-ethoxy-5-oxo-1,2,3,5-tetrahydroindolizine-3-carboxylic acid

To a solution of methyl 2-(5-bromopyridin-2-yl)-3-(1-methyl-1H-pyrazol-3-yl)propanoate (170 mg, 0.43 mmol, 1.0 equiv.) in THF:H₂O:MeOH=2:1:1 (10 mL:5 mL:5 mL) was added lithium hydroxide (21 mg, 0.86 mmol, 2.0 equiv.).

The mixture was stirred at room temperature for 3 h. The pH value of the aqueous phase was adjusted to 5 with HCl solution (1 M), the organic layer was separated, dried, and concentrated under vacuum to yield (3S)-7-(6-amino-3-chloro-2-fluorophenyl)-1-ethoxy-5-oxo-1,2,3,5-tetrahydroindolizine-3-carboxylic acid as a yellow solid, which was used in the next step without further purification. LC/MS (ES, m/z): mass calculated for C₁₇H₁₆ClFN₂O₄: 366.08, measured: 367.05 [M+H]⁺.

### Step 6: (3S)-7-(3-Chloro-2-fluoro-6-(1H-tetrazol-1-yl)phenyl)-1-ethoxy-5-oxo-1,2,3,5-tetrahydroindolizine-3-carboxylic acid

To a solution of (3S)-7-(6-amino-3-chloro-2-fluorophenyl)-1-ethoxy-5-oxo-1,2,3,5-tetrahydroindolizine-3-carboxylic acid (150 mg, 0.41 mmol, 1.0 equiv.) in acetic acid (15 mL) was added trimethoxymethane (434 mg, 4.09 mmol, 10.0 equiv.), and TMSN₃ (471 mg, 4.09 mmol, 10.0 equiv.). The resulting mixture was stirred overnight at room temperature, then concentrated under vacuum. The residue was washed with toluene and concentrated. The resulting residue was purified by reverse column chromatography on C18 column with CH₃CN/0.05% TFA water (5%→80%) to yield (3S)-7-(3-chloro-2-fluoro-6-(1H-tetrazol-1-yl)phenyl)-1-ethoxy-5-oxo-1,2,3,5-tetrahydroindolizine-3-carboxylic acid as a light yellow solid. LC/MS (ES, m/z): mass calculated for C₁₈H₁₅ClFN₅O₄: 419.08, measured: 420.05 [M+H]+.

### Step 7: 2-(6-Amino-2-fluoropyridin-3-yl)-2-oxoethyl 7-(3-chloro-2-fluoro-6-(1H-tetrazol-1-yl)phenyl)-1-ethoxy-5-oxo-1,2,3,5-tetrahydroindolizine-3-carboxylate

To a solution of 7-(3-chloro-2-fluoro-6-(1H-tetrazol-1-yl)phenyl)-1-ethoxy-5-oxo-1,2,3,5-tetrahydroindolizine-3-carboxylic acid (150 mg, 0.36 mmol, 1.0 equiv.) in acetonitrile (5 mL) was added potassium carbonate (148 mg, 1.07 mmol, 3.0 equiv.). After the reaction mixture was stirred at room temperature for 30 min, then 1-(6-amino-2-fluoropyridin-3-yl)-2-bromoethan-1-one (108 mg, 0.46 mmol, 1.3 equiv.) was added. The reaction mixture was stirred for 2 h at room temperature The reaction mixture was concentrated under vacuum and the residue was purified by silica gel column chromatography (0→5% CH₃OH/CH₂Cl₂) to yield 2-(6-amino-2-fluoropyridin-3-yl)-2-oxoethyl 7-(3-chloro-2-fluoro-6-(1H-tetrazol-1-yl)phenyl)-1-ethoxy-5-oxo-1,2,3,5-tetrahydroindolizine-3-carboxylate as a light brown solid. LC/MS (ES, m/z): mass calculated for C₂₅H₂₀ClF₂N₇O₅: 571.12, measured: 572.20 [M+H]⁺.

### Step 8: (1R,3S)-3-(5-(6-amino-2-fluoropyridin-3-yl)-1H-imidazol-2-yl)-7-(3-chloro-2-fluoro-6-(1H-tetrazol-1-yl)phenyl)-1-ethoxy-2,3-dihydroindolizin-5(1H)-one

To a solution of 2-(6-amino-2-fluoropyridin-3-yl)-2-oxoethyl 7-(3-chloro-2-fluoro-6-(1H-tetrazol-1-yl)phenyl)-1-ethoxy-5-oxo-1,2,3,5-tetrahydroindolizine-3-carboxylate (140 mg, 0.25 mmol, 1.0 equiv.) in toluene (20 mL) was added ammonium acetate (283 mg, 3.67 mmol, 15.0 equiv.) followed by addition of acetic acid (147 mg, 2.45 mmol, 10.0 equiv.). The reaction mixture was stirred at 100° C. for 1 h, concentrated under vacuum and the residue purified by silica gel chromatography with MeOH/DCM (0-20%) to yield 3-(5-(6-amino-2-fluoropyridin-3-yl)-1H-imidazol-2-yl)-7-(3-chloro-2-fluoro-6-(1H-tetrazol-1-yl)phenyl)-1-ethoxy-2,3-dihydroindolizin-5(1H)-one as a light yellow solid. The racemic mixture was further purified by prep-chiral-HPLC with Hex (0.1% DEA):EtOH=50:50 to yield (1R,3S)-3-(5-(6-amino-2-fluoropyridin-3-yl)-1H-imidazol-2-yl)-7-(3-chloro-2-fluoro-6-(1H-tetrazol-1-yl)phenyl)-1-ethoxy-2,3-dihydroindolizin-5(1H)-one as a light yellow solid.

LC/MS (ES, m/z): mass calculated for C₂₅H₂₀ClF₂N₉O₂: 551.14, measured: 552.15 [M+H]⁺. ¹H NMR (300 MHz, Methanol-d₄) δ 9.41 (s, 1H), 7.99-7.84 (m, 2H), 7.60 (dd, J=8.7, 1.7 Hz, 1H), 7.32 (d, J=2.9 Hz, 1H), 6.49 (dd, J=8.3, 1.9 Hz, 1H), 6.36 (d, J=5.4 Hz, 2H), 5.86 (dd, J=8.3, 4.8 Hz, 1H), 5.26 (t, J=6.2 Hz, 1H), 3.67-3.54 (m, 2H), 2.90-2.76 (m, 1H), 2.64 (ddd, J=13.7, 8.3, 5.9 Hz, 1H), 1.25 (t, J=7.0 Hz, 3H).

### Example 140: (6R*,8R*)-2-(3-Chloro-2-fluoro-6-(1H-tetrazol-1-yl)phenyl)-8-methyl-6-(5-(1-methyl-1H-pyrazol-5-yl)-1H-imidazol-2-yl)-7,8-dihydropyrrolo[1,2-a]pyrimidin-4(6H)-one

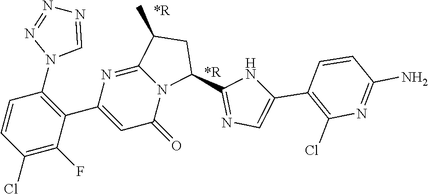 [SMILES: C[C@H]1C[C@@H](c2ncc(-c3ccc(N)nc3Cl)[nH]2)n2c1nc(-c1c(-n3cnnn3)ccc(Cl)c1F)cc2=O]

LC/MS: mass calculated for C₂₃H₁₇Cl₂FN₁₀O: 538.09, measured: 539.10 [M+H]⁺. ¹H NMR (400 MHz, Methanol-d₄) δ 9.47 (s, 1H), 7.76-8.00 (m, 2H), 7.61 (dd, J=8.7, 1.7 Hz, 1H), 7.37 (s, 1H), 6.64 (d, J=2.3 Hz, 1H), 6.56 (d, J=8.5 Hz, 1H), 5.60-5.70 (m, 1H), 3.45-3.56 (m, 1H), 2.58-2.68 (m, 1H), 2.20-2.32 (m, 1H), 1.08-1.11 (m, 3H).

### Example 141: (6R,8S)-2-(3-Chloro-2-fluoro-6-(1H-tetrazol-1-yl)phenyl)-8-methyl-6-(5-(1-methyl-1H-pyrazol-5-yl)-1H-imidazol-2-yl)-7,8-dihydropyrrolo[1,2-a]pyrimidin-4(6H)-one

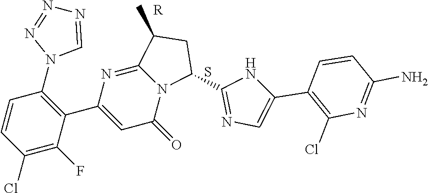

LC/MS: mass calculated for C₂₃H₁₇Cl₂FN₁₀O: 538.09, measured: 539.10 [M+H]⁺. ¹H NMR (400 MHz, Methanol-d₄) δ 9.47 (s, 1H), 7.80-7.90 (m, 2H), 7.61 (dd, J=8.6, 1.6 Hz, 1H), 7.36 (s, 1H), 6.64 (d, J=2.3 Hz, 1H), 6.57 (d, J=8.5 Hz, 1H), 5.70-5.80 (m, 1H), 3.50-3.60 (m, 1H), 2.60-2.70 (m, 1H), 2.18-2.31 (m, 1H), 1.09-1.12 (m, 3H).

### Example 142: (6S,8S)-2-(3-Chloro-2-fluoro-6-(1H-tetrazol-1-yl)phenyl)-8-methyl-6-(5-(1-methyl-1H-pyrazol-5-yl)-1H-imidazol-2-yl)-7,8-dihydropyrrolo[1,2-a]pyrimidin-4(6H)-one

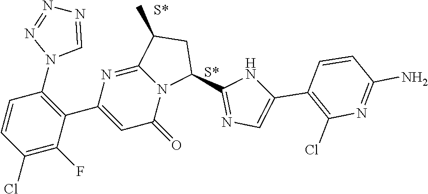 [SMILES: C[C@H]1C[C@@H](c2ncc(-c3ccc(N)nc3Cl)[nH]2)n2c1nc(-c1c(-n3cnnn3)ccc(Cl)c1F)cc2=O]

### Step 1: 2-(6-Amino-2-chloropyridin-3-yl)-2-oxoethyl 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate

To a mixture of 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylic acid (300 mg, 0.76 mmol, 1.0 equiv) and potassium carbonate (265 mg, 1.91 mmol, 2.5 equiv) in CH₃CN (1 mL) was added 1-(6-amino-2-chloropyridin-3-yl)-2-bromoethanone (383 mg, 1.53 mmol, 2.0 equiv). The reaction mixture was stirred at 35° C. for 2 h. The solvent was removed under vacuum and the residue was purified by flash column chromatography on silica gel (MeOH/DCM, 0-5%) to yield 2-(6-amino-2-chloropyridin-3-yl)-2-oxoethyl 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate as a yellow solid. LC/MS: mass calculated for C₂₃H₁₇Cl₂FN₈O₄: 559.337, measured: 581.05 [M+Na]⁺.

### Step 2: (6*S,8*S)-2-(3-Chloro-2-fluoro-6-(1H-tetrazol-1-yl)phenyl)-8-methyl-6-(5-(1-methyl-1H-pyrazol-5-yl)-1H-imidazol-2-yl)-7,8-dihydropyrrolo[1,2-a]pyrimidin-4(6H)-one

To a mixture of 2-(6-amino-2-chloropyridin-3-yl)-2-oxoethyl 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate (260 mg, 0.46 mmol, 1.0 equiv) and ammonium acetate (358 mg, 4.64 mmol, 10.0 equiv) in toluene (5 mL) was added acetic acid (2.791 mg, 0.046 mmol, 0.1 equiv). The reaction mixture was stirred at 90° C. for 2.0 h, concentrated and the residue was purified by reverse phase chromatography on C18 (120 g, CH₃CN/H₂O (0.05% CF₃COOH): 0>>>60%) to yield 6-(5-(6-amino-2-chloropyridin-3-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one as a white solid. The racemic mixture was separated by prep-chiral-HPLC separation. The fractions were combined and concentrated under vacuum to yield (6S*,8S*)-6-(5-(6-amino-2-chloropyridin-3-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one as a white solid.

LC/MS: mass calculated for C₂₃H₁₇Cl₂FN₁₀O: 538.09, measured: 539.10 [M+H]⁺. ¹H NMR (400 MHz, Methanol-d₄) δ 9.45 (s, 1H), 7.85-7.92 (m, 2H), 7.58-7.63 (m, 1H), 7.30-7.40 (m, 1H), 6.55-6.61 (m, 2H), 5.50-5.61 (m, 1H), 3.10-3.20 (m, 1H), 2.80-2.92 (m, 1H), 2.00-2.10 (m, 1H), 1.20-1.27 (m, 3H).

### Example 143: (6R*,8R*)-2-(3-Chloro-2-fluoro-6-(1H-tetrazol-1-yl)phenyl)-8-methyl-6-(5-(1-methyl-1H-pyrazol-5-yl)-1H-imidazol-2-yl)-7,8-dihydropyrrolo[1,2-a]pyrimidin-4(6H)-one

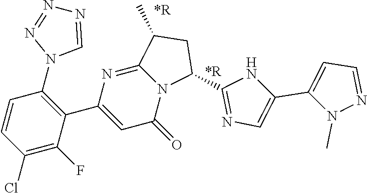 [SMILES: C[C@@H]1C[C@H](c2ncc(-c3ccnn3C)[nH]2)n2c1nc(-c1c(-n3cnnn3)ccc(Cl)c1F)cc2=O]

LC/MS: mass calculated for C₂₂H₁₈ClFN₁₀O: 492.13, measured: 493.15 [M+H]⁺. ¹H NMR (400 MHz, DMSO-d₆) δ 9.70 (s, 1H), 8.01 (t, J=8.2 Hz, 1H), 7.68-7.72 (m, 1H), 7.43 (s, 1H), 7.32 (d, J=2.0 Hz, 1H), 6.60 (d, J=2.0 Hz, 1H), 6.37 (d, J=1.9 Hz, 1H), 5.62 (d, J=8.6 Hz, 1H), 3.91 (s, 3H), 3.30-3.40 (m, 1H), 2.30-2.40 (m, 1H), 2.10-2.20 (m, 1H), 0.90-1.00 (m, 3H).

### Example 144: (6S*,8S*)-2-(3-Chloro-2-fluoro-6-(1H-tetrazol-1-yl)phenyl)-8-methyl-6-(5-(1-methyl-1H-pyrazol-5-yl)-1H-imidazol-2-yl)-7,8-dihydropyrrolo[1,2-a]pyrimidin-4(6H)-one

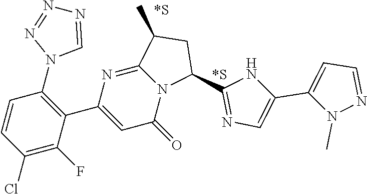 [SMILES: C[C@H]1C[C@@H](c2ncc(-c3ccnn3C)[nH]2)n2c1nc(-c1c(-n3cnnn3)ccc(Cl)c1F)cc2=O]

LC/MS: mass calculated for C₂₂H₁₈ClFN₁₀O: 492.13, measured: 493.15 [M+H]⁺. ¹H NMR (400 MHz, DMSO-d₆) δ 9.57 (d, J=2.3 Hz, 1H), 7.89-7.92 (m, 1H), 7.59-7.62 (m, 1H), 7.30-7.48 (m, 2H), 6.57 (d, J=2.1 Hz, 1H), 6.38 (s, 1H), 5.58-5.63 (m, 1H), 3.86 (s, 3H), 3.28-3.36 (m, 1H), 2.30-2.40 (s, 1H), 2.07-2.19 (m, 1H), 0.89-0.95 (m, 3H).

### Example 145: (6S*,8R*)-2-(3-Chloro-2-fluoro-6-(1H-tetrazol-1-yl)phenyl)-8-methyl-6-(5-(1-methyl-1H-pyrazol-5-yl)-1H-imidazol-2-yl)-7,8-dihydropyrrolo[1,2-a]pyrimidin-4(6H)-one

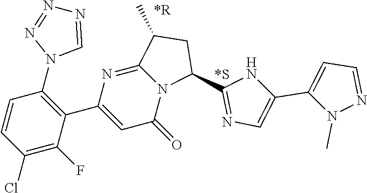 [SMILES: C[C@@H]1C[C@@H](c2ncc(-c3ccnn3C)[nH]2)n2c1nc(-c1c(-n3cnnn3)ccc(Cl)c1F)cc2=O]

LC/MS: mass calculated for C₂₂H₁₈ClFN₁₀O: 492.13, measured: 493.15 [M+H]⁺. ¹H NMR (400 MHz, DMSO-d₆) δ 9.47 (s, 1H), 7.91 (t, J=8.1 Hz, 1H), 7.62 (dd, J=8.7, 1.7 Hz, 1H), 7.32-7.52 (m, 2H), 6.48-6.62 (m, 2H), 5.58-5.61 (m, 1H), 4.01 (s, 3H), 3.15-3.20 (m, 1H), 2.80-2.89 (m, 1H), 2.00-2.12 (m, 1H), 1.18-1.36 (m, 3H).

### Example 146: (6*S,8*S)-2-(3-Chloro-2-fluoro-6-(1H-tetrazol-1-yl)phenyl)-8-methyl-6-(5-(1-methyl-1H-pyrazol-5-yl)-1H-imidazol-2-yl)-7,8-dihydropyrrolo[1,2-a]pyrimidin-4(6H)-one

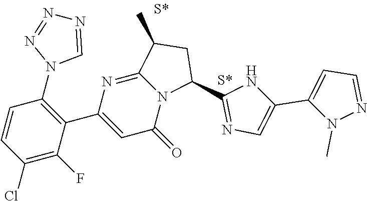 [SMILES: C[C@H]1C[C@@H](c2ncc(-c3ccnn3C)[nH]2)n2c1nc(-c1c(-n3cnnn3)ccc(Cl)c1F)cc2=O]

### Step 1: 2-(1-Methyl-1H-pyrazol-5-yl)-2-oxoethyl 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate

To a mixture of 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylic acid (300 mg, 0.76 mmol, 1.0 equiv) and potassium carbonate (265 mg, 1.91 mmol, 2.5 equiv) in CH₃CN (1 mL) was added 2-bromo-1-(1-methyl-1H-pyrazol-5-yl)ethanone (233.820 mg, 1.152 mmol, 1.5 equiv). The resulting reaction mixture was stirred at 35° C. for 2 h. The solvent was removed and the residue was purified by flash column chromatography on silica gel (MeOH/DCM, 0-5%) to yield 2-(1-methyl-1H-pyrazol-5-yl)-2-oxoethyl 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate as a yellow solid. LC/MS: mass calculated, for C₂₂H₁₈ClFN₈O₄: 512.88, measured: 513.05 [M+H]⁺.

### Step 2: (6*S,8*S)-2-(3-Chloro-2-fluoro-6-(1H-tetrazol-1-yl)phenyl)-8-methyl-6-(5-(1-methyl-1H-pyrazol-5-yl)-1H-imidazol-2-yl)-7,8-dihydropyrrolo[1,2-a]pyrimidin-4(6H)-one

To a mixture of 2-(1-methyl-1H-pyrazol-5-yl)-2-oxoethyl 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate (300 mg, 0.58 mmol, 1.0 equiv) and ammonium acetate (450 mg, 5.84 mmol, 10.0 equiv) in toluene (6 mL) was added acetic acid (0.6 mL). The reaction mixture was stirred at 90° C. for 1.5 h, then concentrated and the residue was purified by reverse phase chromatography on C18 (120 g, CH₃CN/H₂O (0.05% CF₃COOH): 0>>>60%) to yield 2-(3-chloro-2-fluoro-6-(1H-tetrazol-1-yl)phenyl)-8-methyl-6-(5-(1-methyl-1H-pyrazol-5-yl)-1H-imidazol-2-yl)-7,8-dihydropyrrolo[1,2-a]pyrimidin-4(6H)-one as a white solid. The racemic mixture was separated by prep-HPLC and prep-chiral-HPLC separation. The fractions were combined and concentrated under vacuum to yield (6S*,8S*)-2-(3-chloro-2-fluoro-6-(1H-tetrazol-1-yl)phenyl)-8-methyl-6-(5-(1-methyl-1H-pyrazol-5-yl)-1H-imidazol-2-yl)-7,8-dihydropyrrolo[1,2-a]pyrimidin-4(6H)-one as a white solid.

LC/MS: mass calculated for C₂₂H₁₈ClFN₁₀O: 492.13, measured: 493.15 [M+H]⁺. ¹H NMR (400 MHz, DMSO-d₆) δ 9.64 (s, 1H), 7.98 (t, J=8.2 Hz, 1H), 7.68 (d, J=8.7 Hz, 1H), 7.27-7.48 (m, 2H), 6.51-6.56 (m, 1H), 6.41 (d, J=2.0 Hz, 1H), 5.52 (dd, J=8.8, 5.6 Hz, 1H), 3.95 (s, 3H), 3.02-3.11 (m, 1H), 2.71-2.83 (m, 1H), 1.80-1.90 (m, 1H), 1.00-1.10 (m, 3H).

### Example 147: (6S*,8R*)-2-(3-Chloro-2-fluoro-6-(1H-tetrazol-1-yl)phenyl)-8-methyl-6-(5-(2-(trifluoromethyl)pyridin-4-yl)-1H-imidazol-2-yl)-7,8-dihydropyrrolo[1,2-a]pyrimidin-4(6H)-one

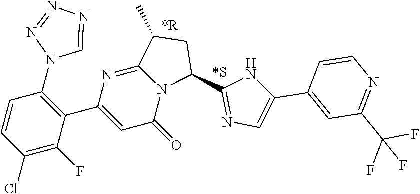

LC/MS: mass calculated for C₂₄H₁₆ClF₄N₉O: 557.11, measured: 580.10 [M+Na]⁺. ¹H NMR (400 MHz, DMSO-d₆) δ 9.49 (s, 1H), 8.61 (d, J=5.1 Hz, 1H), 8.14 (s, 1H), 7.98 (d, J=5.1 Hz, 1H), 7.80-7.90 (m, 2H), 7.63 (dd, J=8.7, 1.6 Hz, 1H), 6.65 (d, J=2.3 Hz, 1H), 5.70-5.80 (m, 1H), 3.50-3.60 (m, 1H), 2.60-2.70 (m, 1H), 2.18-2.30 (m, 1H), 1.10-1.18 (m, 3H).

### Example 148: (6S*,8S*)-2-(3-Chloro-2-fluoro-6-(1H-tetrazol-1-yl)phenyl)-8-methyl-6-(5-(2-(trifluoromethyl)pyridin-4-yl)-1H-imidazol-2-yl)-7,8-dihydropyrrolo[1,2-a]pyrimidin-4(6H)-one

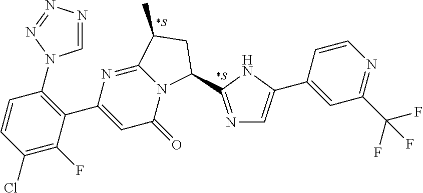 [SMILES: C[C@H]1C[C@@H](c2ncc(-c3ccnc(C(F)(F)F)c3)[nH]2)n2c1nc(-c1c(-n3cnnn3)ccc(Cl)c1F)cc2=O]

LC/MS: mass calculated for C₂₄H₁₆ClF₄N₉O: 557.11, measured: 580.10 [M+Na]⁺. ¹H NMR (400 MHz, DMSO-d₆) δ 9.70 (s, 1H), 8.63 (d, J=5.2 Hz, 1H), 8.09 (s, 1H), 7.96-8.05 (m, 2H), 7.93 (d, J=5.2 Hz, 1H), 7.71 (dd, J=8.7, 1.4 Hz, 1H), 6.61 (d, J=2.0 Hz, 1H), 5.60-5.66 (m, 1H), 3.40-3.50 (m, 1H), 2.35-2.45 (m, 1H), 2.12-2.20 (m, 1H), 0.92-1.02 (m, 3H).

### Example 149: (6*S,8*S)-6-(5-(6-Amino-2-fluoropyridin-3-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

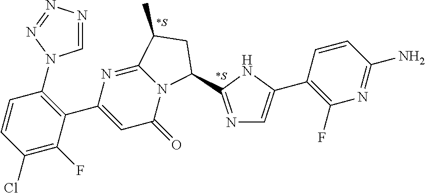 [SMILES: C[C@H]1C[C@@H](c2ncc(-c3ccc(N)nc3F)[nH]2)n2c1nc(-c1c(-n3cnnn3)ccc(Cl)c1F)cc2=O]

### Step 1: Ethyl (4R)-5-methoxy-4-methyl-3,4-dihydro-2H-pyrrole-2-carboxylate

To a solution of ethyl 4-methyl-5-oxopyrrolidine-2-carboxylate (6.0 g, 35.48 mmol, 1.0 equiv.) in CH₂Cl₂ (80 mL) was added trimethyloxonium tetrafluoroborate (7.6 g, 52.57 mmol, 1.5 equiv.). The resulting mixture was stirred at room temperature. for 3 h. The reaction was quenched with saturated aqueous solution of sodium hydrogen carbonate (150 mL). The resulting mixture was extracted with CH₂Cl₂ (3×100 mL). The organic layers were combined, dried over Na₂SO₄, filtered, and concentrated to yield ethyl 5-methoxy-4-methyl-3,4-dihydro-2H-pyrrole-2-carboxylate as a light brown oil.

### Step 2: Ethyl 5-amino-4-methyl-3,4-dihydro-2H-pyrrole-2-carboxylate

To a solution of ethyl 5-methoxy-4-methyl-3,4-dihydro-2H-pyrrole-2-carboxylate (5.4 g, 29.16 mmol, 1.0 equiv.) in ethanol (60 mL) was added ammonium chloride (2.3 g, 43.73 mmol, 1.5 equiv.). The resulting mixture was stirred at 80° C. for 3 h and concentrated to yield ethyl 5-amino-4-methyl-3,4-dihydro-2H-pyrrole-2-carboxylate as a yellow solid.

### Step 3: Ethyl 2-hydroxy-8-methyl-4-oxo-4,6,7,8-tetrahydropyrrolo[1,2-a]pyrimidine-6-carboxylate

To a mixture of ethyl 5-amino-4-methyl-3,4-dihydro-2H-pyrrole-2-carboxylate (8.6 g, 50.53 mmol, 1.0 equiv.) in 1,4-dioxane (120 mL) and triethylamine (21.1 mL, 151.58 mmol, 3.0 equiv.) was added ethyl 3-chloro-3-oxopropanoate (6.4 mL, 50.52 mmol, 1.0 equiv.) at 0° C. The reaction was stirred at 80° C. for 3 h. The combined organic was concentrated and the residue was purified by reverse phase chromatography on C18 (330 g, MeCN/H₂O (0.05% CF₃COOH): 0-60%) to yield ethyl 2-hydroxy-8-methyl-4-oxo-4,6,7,8-tetrahydropyrrolo[1,2-a]pyrimidine-6-carboxylate as a yellow oil. LC/MS: mass calculated for C₁₁H₁₄N₂O₄: 238.24, measured: 239.05 [M+H]⁺.

### Step 4: Ethyl 8-methyl-4-oxo-2-(((trifluoromethyl)sulfonyl)oxy)-4,6,7,8-tetrahydropyrrolo[1,2-a]pyrimidine-6-carboxylate

To a mixture of ethyl 2-hydroxy-8-methyl-4-oxo-4,6,7,8-tetrahydropyrrolo[1,2-a]pyrimidine-6-carboxylate (3.5 g, 14.69 mmol, 1.0 equiv.) in DMF (50 mL) and triethylamine (4.1 mL, 29.38 mmol, 3.0 equiv.) was added trifluoro-N-phenyl-N-(trifluoromethylsulfonyl)methane sulfonamide (6.8 g, 19.10 mmol, 1.3 equiv.) at 0° C. The reaction was stirred at room temperature for 3 h. Water was added, the mixture was extracted with ethyl acetate. The combined extracts were washed with water, saturated brine and dried over anhydrous Na₂SO₄. The combined organic was concentrated and the residue was purified by reverse phase chromatography on C18 (330 g, MeCN/H₂O (0.05% CF3COOH): 0-60%) to yield ethyl 8-methyl-4-oxo-2-(((trifluoromethyl)sulfonyl)oxy)-4,6,7,8-tetrahydropyrrolo[1,2-a]pyrimidine-6-carboxylate as a yellow oil. LC/MS: mass calculated for C₁₂H₁₃F₃N₂O₆S: 370.30, measured: 371.00 [M+H]⁺.

### Step 5: Ethyl 2-(6-amino-3-chloro-2-fluorophenyl)-8-methyl-4-oxo-4,6,7,8-tetrahydropyrrolo[1,2-a]pyrimidine-6-carboxylate

To a mixture of ethyl 8-methyl-4-oxo-2-(trifluoromethylsulfonyloxy)-4,6,7,8-tetrahydropyrrolo[1,2-a]pyrimidine-6-carboxylate (1.5 g, 4.05 mmol, 1.0 equiv.) in 1,4-dioxane (30 mL) and 6-amino-3-chloro-2-fluorophenylboronic acid (1.2 g, 6.08 mmol, 1.5 equiv.) and cesium fluoride (1.2 g, 8.10 mmol, 2.0 equiv.) was added tetrakis(triphenylphosphine)palladium (468 mg, 0.41 mmol, 0.1 equiv.) under N₂. The reaction mixture was stirred at 100° C. for 2 h, diluted with water, and extracted with ethyl acetate. The combined extracts were washed with water, brine, and dried over anhydrous Na₂SO₄ and concentrated. The residue was purified by flash column chromatography on silica gel with MeOH/DCM (0-2%) to yield ethyl 2-(6-amino-3-chloro-2-fluorophenyl)-8-methyl-4-oxo-4,6,7,8-tetrahydropyrrolo[1,2-a]pyrimidine-6-carboxylate as a yellow solid. LC/MS: mass calculated for C₁₇H₁₇ClFN₃O₃: 365.79, measured: 366.00 [M+H]⁺.

### Step 6: 2-(6-Amino-3-chloro-2-fluorophenyl)-8-methyl-4-oxo-4,6,7,8-tetrahydropyrrolo[1,2-a]pyrimidine-6-carboxylic acid

To a mixture of ethyl 2-(6-amino-3-chloro-2-fluorophenyl)-8-methyl-4-oxo-4,6,7,8-tetrahydropyrrolo[1,2-a]pyrimidine-6-carboxylate (1.5 g, 4.10 mmol, 1.0 equiv.) in methanol (10 mL) and THE (20 mL) was added lithium hydroxide (10 mL, 2 M). The reaction was mixture was stirred at room temperature. for 3 h and adjusted to pH 5-7, then extracted with ethyl acetate. The combined extracts were washed with water, saturated brine and dried over anhydrous Na₂SO₄. The filtrate was concentrated to yield 2-(6-amino-3-chloro-2-fluorophenyl)-8-methyl-4-oxo-4,6,7,8-tetrahydropyrrolo[1,2-a]pyrimidine-6-carboxylic acid as a yellow solid. LC/MS: mass calculated, for C₁₅H₁₃ClFN₃O₃: 337.73, measured: 338.00 [M+H]⁺.

### Step 7: 2-(3-Chloro-2-fluoro-6-(1H-tetrazol-1-yl)phenyl)-8-methyl-4-oxo-4,6,7,8-tetrahydropyrrolo[1,2-a]pyrimidine-6-carboxylic acid

A mixture of 2-(6-amino-3-chloro-2-fluorophenyl)-8-methyl-4-oxo-4,6,7,8-tetrahydropyrrolo[1,2-a]pyrimidine-6-carboxylic acid (1.1 g, 3.26 mmol, 1.00 equiv.), azidotrimethylsilane (1.9 g, 16.29 mmol, 5.0 equiv.) and trimethoxymethane (3.5 g, 32.57 mmol, 10.0 equiv.) in acetic acid (15 mL) was stirred overnight at room temperature. The mixture was concentrated and the residue was purified by reverse phase chromatography on C18 (120 g, CH₃CN/H₂O (0.05% CF₃COOH): 0-60%) to yield 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylic acid as a white solid. LC/MS: mass calculated, for C₁₆H₁₂ClFN₆O₃: 390.76, measured: 391.05 [M+H]⁺.

### Step 8: 2-(6-Amino-2-fluoropyridin-3-yl)-2-oxoethyl 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate

To a solution of 7-(3-chloro-2-fluoro-6-(1H-tetrazol-1-yl)phenyl)-1-methylene-5-oxo-1,2,3,5-tetrahydroindolizine-3-carboxylic acid (200 mg, 0.52 mmol, 1.0 equiv) and potassium carbonate (100 mg, 0.77 mmol, 1.5 equiv) in CH₃CN (10 mL) was added N-(4-(2-bromoacetyl)pyridin-2-yl)acetamide (190 mg, 0.77 mmol, 1.5 eq.). The reaction mixture was stirred at 30° C. for 4 h, quenched with water, then extracted with ethyl acetate. The combined organic layer was washed with brine, dried over Na₂SO₄. The solids were filtered out. The resulting organic phase was concentrated under vacuum. The residue was applied onto a silica gel column (MeOH/DCM) to yield 2-(6-amino-2-fluoropyridin-3-yl)-2-oxoethyl 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate as a yellow solid. LC/MS: mass calculated for C₂₆H₁₉ClFN₇O₅: 563.92, measured: 564.10 [M+H]⁺.

### Step 9: (6*S,8*S)-6-(5-(6-Amino-2-fluoropyridin-3-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

To a mixture of 2-(6-amino-2-fluoropyridin-3-yl)-2-oxoethyl 2-(3-chloro-2-fluoro-6-(1H-tetrazol-1-yl)phenyl)-8-methyl-4-oxo-4,6,7,8-tetrahydropyrrolo[1,2-a]pyrimidine-6-carboxylate (210 mg, 0.38 mmol, 1.0 equiv) and ammonium acetate (298 mg, 3.86 mmol, 10.0 equiv) in toluene (6 mL) was added acetic acid (0.2 ml). The reaction was stirred at 90° C. for 1.5 h. The mixture was concentrated and the residue purified by reverse phase chromatography on C18 (120 g, CH₃CN/H₂O (0.05% CF₃COOH): 0>>>60%) to yield 6-(5-(6-amino-2-fluoropyridin-3-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one as a white solid. The racemic mixture was separated by prep-HPLC and prep-chiral-HPLC separation. The fractions were collected, combined, and concentrated under vacuum to yield (6S*,8S*)-6-(5-(6-amino-2-fluoropyridin-3-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one as a white solid.

LC/MS: mass calculated for C₂₃H₁₇ClF₂N₁₀O: 522.12, measured: 523.20 [M+H]⁺. ¹H NMR (400 MHz, DMSO-d₆) δ 9.64 (s, 1H), 7.94-8.06 (m, 2H), 7.69 (dd, J=8.7, 1.4 Hz, 1H), 7.13 (d, J=3.9 Hz, 1H), 6.52 (d, J=2.0 Hz, 1H), 6.40 (dd, J=8.2, 2.2 Hz, 1H), 5.49 (dd, J=8.6, 5.8 Hz, 1H), 3.00-3.07 (m, 1H), 2.70-2.81 (m, 1H), 1.80-1.90 (m, 1H), 1.00-1.09 (m, 3H).

### Example 150: (6S*,8S*)-6-(5-(6-Amino-2-fluoropyridin-3-yl)-1H-imidazol-2-yl)-2-(3-chloro-2-fluoro-6-(1H-tetrazol-1-yl)phenyl)-8-methyl-7,8-dihydropyrrolo[1,2-a]pyrimidin-4(6H)-one

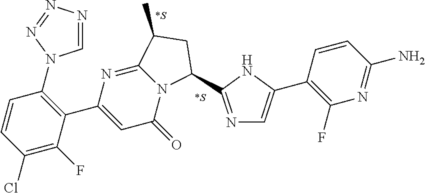 [SMILES: C[C@H]1C[C@@H](c2ncc(-c3ccc(N)nc3F)[nH]2)n2c1nc(-c1c(-n3cnnn3)ccc(Cl)c1F)cc2=O]

LC/MS: mass calculated for C₂₃H₁₇ClF₂N₁₀O: 522.12, measured: 523.20 [M+H]⁺. ¹H NMR (400 MHz, DMSO-d₆) δ 9.69 (s, 1H), 7.75-8.15 (m, 3H), 7.12 (d, J=3.9 Hz, 1H), 6.59 (d, J=2.0 Hz, 1H), 6.34-6.44 (m, 1H), 5.60 (dd, J=8.9, 1.3 Hz, 1H), 3.35-3.46 (m, 1H), 2.30-2.42 (m, 1H), 2.07-2.20 (m, 1H), 0.90-1.00 (m, 3H).

### Example 151: (1*R,3*R)-3-(5-(6-Amino-2-fluoropyridin-3-yl)-1H-imidazol-2-yl)-7-(3-chloro-2-fluoro-6-(1H-tetrazol-1-yl)phenyl)-1-methoxy-2,3-dihydroindolizin-5(1H)-one

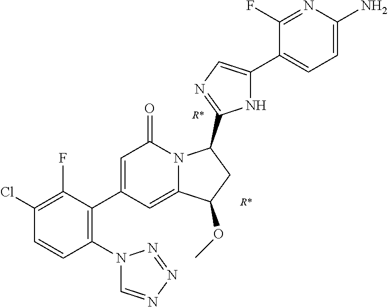 [SMILES: CO[C@@H]1C[C@H](c2ncc(-c3ccc(N)nc3F)[nH]2)n2c1cc(-c1c(-n3cnnn3)ccc(Cl)c1F)cc2=O]

### Step 1: Methyl (S)-7-(6-((tert-butoxycarbonyl)amino)-3-chloro-2-fluorophenyl)-5-oxo-1,2,3,5-tetrahydroindolizine-3-carboxylate

A mixture of methyl (S)-7-(6-amino-3-chloro-2-fluorophenyl)-5-oxo-1,2,3,5-tetrahydroindolizine-3-carboxylate (2.0 g, 5.94 mmol, 1.0 equiv.) and (Boc)₂O (8.9 mL, 29.70 mmol, 5.0 equiv.) in toluene (20 mL) was heated at reflux overnight. The reaction solution was concentrated and the residue was purified by silica gel chromatography (0→60% EA/PE) to yield methyl (S)-7-(6-((tert-butoxycarbonyl)amino)-3-chloro-2-fluorophenyl)-5-oxo-1,2,3,5-tetrahydroindolizine-3-carboxylate as a yellow solid.

### Step 2: Methyl (3S)-7-(6-((tert-butoxycarbonyl)amino)-3-chloro-2-fluorophenyl)-1-hydroxy-5-oxo-1,2,3,5-tetrahydroindolizine-3-carboxylate

To a mixture of methyl 7-(6-((tert-butoxycarbonyl)amino)-3-chloro-2-fluorophenyl)-5-oxo-1,2,3,5-tetrahydroindolizine-3-carboxylate (1.8 g, 4.12 mmol, 1.0 equiv.) in 1,4-dioxane (50 mL) was added SeO₂ (3.6 g, 32.43 mmol, 8.0 equiv.). The mixture was stirred at 100° C. for 3 h under N₂. The mixture was concentrated under vacuum. The residue was applied on a silica gel column (0→50% EA/PE) to yield methyl 7-(6-((tert-butoxycarbonyl)amino)-3-chloro-2-fluorophenyl)-1-hydroxy-5-oxo-1,2,3,5-tetrahydroindolizine-3-carboxylate as a yellow solid. LC/MS: mass calculated for C₂₁H₂₂ClFN₂O₆: 452.12, measured: 453.20 [M+H]⁺.

### Step 3: Methyl (3S)-7-(6-((tert-butoxycarbonyl)amino)-3-chloro-2-fluorophenyl)-1-methoxy-5-oxo-1,2,3,5-tetrahydroindolizine-3-carboxylate

To a solution of methyl 7-(6-(bis(tert-butoxycarbonyl)amino)-3-chloro-2-fluorophenyl)-1-hydroxy-5-oxo-1,2,3,5-tetrahydroindolizine-3-carboxylate (1.0 g, 1.80 mmol, 1.0 equiv.) in CHCl₃ (20 mL) was added silver oxide (2.24 g, 18.07 mmol, 10.0 equiv.) and iodomethane (2.6 g, 18.07 mmol, 10.0 equiv.). The mixture stirred at 50° C. for 24 h, cooled to room temperature, filtered through a pad of CELITE. The filtrate was concentrated under vacuum to yield methyl (3S)-7-(6-((tert-butoxycarbonyl)amino)-3-chloro-2-fluorophenyl)-1-methoxy-5-oxo-1,2,3,5-tetrahydroindolizine-3-carboxylate as an yellow solid, which was used in the next step without further purification.

### Step 4: Methyl (3S)-7-(6-amino-3-chloro-2-fluorophenyl)-1-methoxy-5-oxo-1,2,3,5-tetrahydroindolizine-3-carboxylate

To a solution of methyl (3S)-7-(6-((tert-butoxycarbonyl)amino)-3-chloro-2-fluorophenyl)-1-methoxy-5-oxo-1,2,3,5-tetrahydroindolizine-3-carboxylate (620 mg) in dichloromethane (15 mL) was added trifluoroacetic acid (3 mL). The reaction mixture was stirred at room temperature for 1 h, then concentrated under vacuum and the residue was purified by reverse column chromatography with CH₃CN/0.05% TFA water (5%→80%) to yield methyl (3S)-7-(6-amino-3-chloro-2-fluorophenyl)-1-methoxy-5-oxo-1,2,3,5-tetrahydroindolizine-3-carboxylate as a light yellow solid. LC/MS (ES, m/z): mass calculated for C₁₉H₂₀ClFN₂O₄: 394.11, measured: 395.05 [M+H]+.

### Step 5: (3S)-7-(6-Amino-3-chloro-2-fluorophenyl)-1-methoxy-5-oxo-1,2,3,5-tetrahydroindolizine-3-carboxylic acid

To a solution of methyl (3S)-7-(6-amino-3-chloro-2-fluorophenyl)-1-methoxy-5-oxo-1,2,3,5-tetrahydroindolizine-3-carboxylate (450 mg, 1.23 mmol, 1.0 equiv.) in THF:H₂O:MeOH=2:1:1 (10 mL:5 mL:5 mL) was added lithium hydroxide (60 mg, 2.46 mmol, 2.0 equiv.). The mixture was stirred at room temperature for 3 h and adjusted to pH 5 with HCl solution (1 M), then concentrated to yield (3S)-7-(6-amino-3-chloro-2-fluorophenyl)-1-methoxy-5-oxo-1,2,3,5-tetrahydroindolizine-3-carboxylic acid as a yellow solid, which was used in the next step without further purification.

### Step 6: (3S)-7-(3-Chloro-2-fluoro-6-(1H-tetrazol-1-yl)phenyl)-1-methoxy-5-oxo-1,2,3,5-tetrahydroindolizine-3-carboxylic acid

To a solution of (3S)-7-(6-amino-3-chloro-2-fluorophenyl)-1-methoxy-5-oxo-1,2,3,5-tetrahydroindolizine-3-carboxylic acid (400 mg, 1.14 mmol, 1.0 equiv.) in acetic acid (15 mL) was added trimethoxymethane (1.2 g, 11.36 mmol, 10.0 equiv.), followed by addition of azidotrimethylsilane (1.3 g, 11.36 mmol, 10.0 equiv.). The resulting mixture was stirred overnight at room temperature, then concentrated under vacuum. The residue was washed with toluene and concentrated. The resulting residue was purified by reverse column chromatography on C18 with CH₃CN/0.05% TFA water (5%→80%) to yield (3S)-7-(3-chloro-2-fluoro-6-(1H-tetrazol-1-yl)phenyl)-1-methoxy-5-oxo-1,2,3,5-tetrahydroindolizine-3-carboxylic acid as a light yellow solid.

### Step 7: 2-(6-Amino-2-fluoropyridin-3-yl)-2-oxoethyl (3S)-7-(3-chloro-2-fluoro-6-(1H-tetrazol-1-yl)phenyl)-1-methoxy-5-oxo-1,2,3,5-tetrahydroindolizine-3-carboxylate

To a solution of 7-(3-chloro-2-fluoro-6-(1H-tetrazol-1-yl)phenyl)-1-methoxy-5-oxo-1,2,3,5-tetrahydroindolizine-3-carboxylic acid (300 mg, 0.74 mmol, 1.0 equiv.) in CH₃CN (8 mL) was added potassium carbonate (306 mg, 2.22 mmol, 3.0 equiv.) and the mixture was stirred at room temperature for 30 minutes. To the resulting mixture was then added a solution of 1-(6-amino-2-fluoropyridin-3-yl)-2-bromoethanone (258 mg, 1.11 mmol) in CH₃CN (2 mL). The resulting mixture was stirred at room temperature overnight, concentrated and the residue was purified by silica gel column chromatography (0→5% CH₃OH/CH₂Cl₂) to yield 2-(6-amino-2-fluoropyridin-3-yl)-2-oxoethyl 7-(3-chloro-2-fluoro-6-(1H-tetrazol-1-yl)phenyl)-1-methoxy-5-oxo-1,2,3,5-tetrahydroindolizine-3-carboxylate as a light brown solid. LC/MS (ES, m/z): mass calculated for C₂₄H₁₈ClF₂N₇O₅: 557.10, measured: 558.15 [M+H]⁺.

### Step 8: (1S,3R)-3-(5-(6-amino-2-fluoropyridin-3-yl)-1H-imidazol-2-yl)-7-(3-chloro-2-fluoro-6-(1H-tetrazol-1-yl)phenyl)-1-methoxy-2,3-dihydroindolizin-5(1H)-one

To a solution of 2-(6-amino-2-fluoropyridin-3-yl)-2-oxoethyl 7-(3-chloro-2-fluoro-6-(1H-tetrazol-1-yl)phenyl)-1-methoxy-5-oxo-1,2,3,5-tetrahydroindolizine-3-carboxylate (170 mg, 0.31 mmol, 1.0 equiv.) in toluene (8 mL) was added ammonium acetate (235 mg, 3.05 mmol, 10.0 equiv.) and acetic acid (0.8 mL, cat.). The resulting mixture was heated at reflux for 2 h. The reaction mixture was quenched with H₂O (20 mL), extracted with ethyl acetate (3×20 mL) and the organic layers were combined, dried over Na₂SO₄, filtered, and concentrated under reduced pressure. The residue was purified by C18 chromatography (0→50% CH₃CN/H₂O) to yield 3-(5-(6-amino-2-fluoropyridin-3-yl)-1H-imidazol-2-yl)-7-(3-chloro-2-fluoro-6-(1H-tetrazol-1-yl)phenyl)-1-methoxy-2,3-dihydroindolizin-5(1H)-one as a white solid. The racemic mixture (80 mg) was separated by prep-chiral-HPLC separation. The collected fractions were combined and concentrated under vacuum to yield (1S*, 3S*)-3-(5-(6-amino-2-fluoropyridin-3-yl)-1H-imidazol-2-yl)-7-(3-chloro-2-fluoro-6-(1H-tetrazol-1-yl)phenyl)-1-methoxy-2,3-dihydroindolizin-5(1H)-one as a white solid. LC/MS (ES, m/z): mass calculated for C₂₄H₁ClF₂N₉O₂: 537.12, measured: 538.20 [M+H]⁺. ¹H NMR (300 MHz, DMSO-d₆) δ 12.19 (s, 1H), 9.67 (s, 1H), 8.05-7.88 (m, 2H), 7.71 (dd, J=8.7, 1.5 Hz, 1H), 7.07 (s, 1H), 6.37 (dd, J=8.2, 2.2 Hz, 2H), 6.22 (d, J=18.6 Hz, 3H), 5.65 (d, J=8.3 Hz, 1H), 5.25 (t, J=7.4 Hz, 1H), 3.33 (m, 3H), 2.70 (d, J=8.9 Hz, 1H), 2.39 (dt, J=12.5, 8.6 Hz, 1H).

### Example 152: Methyl (4-(2-((1R,3S)-7-(3-chloro-2-fluoro-6-(1H-tetrazol-1-yl)phenyl)-1-methoxy-5-oxo-1,2,3,5-tetrahydroindolizin-3-yl)-1H-imidazol-5-yl)phenyl)carbamate

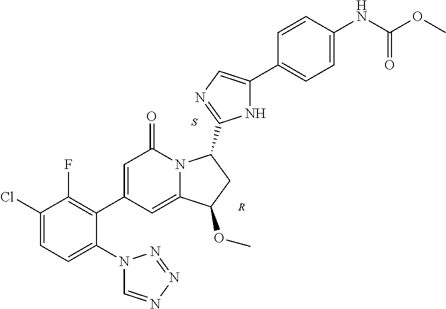

### Step 1: Methyl (S)-7-(6-((tert-butoxycarbonyl)amino)-3-chloro-2-fluorophenyl)-5-oxo-1,2,3,5-tetrahydroindolizine-3-carboxylate

A mixture of methyl (S)-7-(6-amino-3-chloro-2-fluorophenyl)-5-oxo-1,2,3,5-tetrahydroindolizine-3-carboxylate (2.0 g, 5.94 mmol, 1.0 equiv.) and (Boc)₂O (8.9 mL, 29.70 mmol, 5.0 equiv.) in toluene (20 mL) was heated at reflux overnight. The reaction solution was concentrated and purified by silica gel chromatography (0→60% EA/PE) to yield methyl (S)-7-(6-((tert-butoxycarbonyl)amino)-3-chloro-2-fluorophenyl)-5-oxo-1,2,3,5-tetrahydroindolizine-3-carboxylate as a yellow solid.

### Step 2: Methyl (3S)-7-(6-((tert-butoxycarbonyl)amino)-3-chloro-2-fluorophenyl)-1-hydroxy-5-oxo-1,2,3,5-tetrahydroindolizine-3-carboxylate

To a mixture of methyl 7-(6-((tert-butoxycarbonyl)amino)-3-chloro-2-fluorophenyl)-5-oxo-1,2,3,5-tetrahydroindolizine-3-carboxylate (1.8 g, 4.12 mmol, 1.0 equiv.) in 1,4-dioxane (50 mL) was added SeO₂ (3.6 g, 32.43 mmol, 8.0 equiv.). The mixture was stirred at 100° C. for 3 h under N₂. The mixture was concentrated under vacuum. The residue was applied on a silica gel column (0→50% EA/PE) to yield methyl 7-(6-((tert-butoxycarbonyl)amino)-3-chloro-2-fluorophenyl)-1-hydroxy-5-oxo-1,2,3,5-tetrahydroindolizine-3-carboxylate as a yellow solid. LC/MS: mass calculated for C₂₁H₂₂ClFN₂O₆: 452.12, measured: 453.20 [M+H]⁺.

### Step 3: Methyl (3S)-7-(6-((tert-butoxycarbonyl)amino)-3-chloro-2-fluorophenyl)-1-methoxy-5-oxo-1,2,3,5-tetrahydroindolizine-3-carboxylate

To a solution of methyl 7-(6-(bis(tert-butoxycarbonyl)amino)-3-chloro-2-fluorophenyl)-1-hydroxy-5-oxo-1,2,3,5-tetrahydroindolizine-3-carboxylate (1.0 g, 1.80 mmol, 1.0 equiv.) in CHCl₃ (20 mL) was added silver oxide (2.24 g, 18.07 mmol, 10.0 equiv.) and iodomethane (2.6 g, 18.07 mmol, 10.0 equiv.). The mixture stirred at 50° C. for 24 h. The mixture was filtered through a pad of CELITE and the filtrate was concentrated under vacuum to yield methyl (3S)-7-(6-((tert-butoxycarbonyl)amino)-3-chloro-2-fluorophenyl)-1-methoxy-5-oxo-1,2,3,5-tetrahydroindolizine-3-carboxylate as a yellow solid, which was used in the next step without further purification.

### Step 4: Methyl (3S)-7-(6-amino-3-chloro-2-fluorophenyl)-1-methoxy-5-oxo-1,2,3,5-tetrahydroindolizine-3-carboxylate

To a solution of methyl (3S)-7-(6-((tert-butoxycarbonyl)amino)-3-chloro-2-fluorophenyl)-1-methoxy-5-oxo-1,2,3,5-tetrahydroindolizine-3-carboxylate (620 mg) in dichloromethane (15 mL) was added trifluoroacetic acid (3 mL). The reaction mixture was stirred at room temperature for 1 h. The solvent was removed under reduced pressure and the residue was purified by reverse column chromatography with CH₃CN/0.05% TFA water (5%→80%) to yield methyl (3S)-7-(6-amino-3-chloro-2-fluorophenyl)-1-methoxy-5-oxo-1,2,3,5-tetrahydroindolizine-3-carboxylate as a light yellow solid. LC/MS (ES, m/z): mass calculated for C₁₉H₂₀ClFN₂O₄: 394.11, measured: 395.05 [M+H]⁺.

### Step 5: (3S)-7-(6-Amino-3-chloro-2-fluorophenyl)-1-methoxy-5-oxo-1,2,3,5-tetrahydroindolizine-3-carboxylic acid

To a solution of methyl (3S)-7-(6-amino-3-chloro-2-fluorophenyl)-1-methoxy-5-oxo-1,2,3,5-tetrahydroindolizine-3-carboxylate (450 mg, 1.23 mmol, 1.0 equiv.) in THF:H₂O:MeOH=2:1:1 (10 mL:5 mL:5 mL) was added lithium hydroxide (60 mg, 2.46 mmol, 2.0 equiv.). The mixture was stirred at room temperature for 3 h, adjusted to pH 5 with HCl solution (1 M), then concentrated under vacuum to yield (3S)-7-(6-amino-3-chloro-2-fluorophenyl)-1-methoxy-5-oxo-1,2,3,5-tetrahydroindolizine-3-carboxylic acid as a yellow solid, which was used in the next step without further purification.

### Step 6: (3S)-7-(3-Chloro-2-fluoro-6-(1H-tetrazol-1-yl)phenyl)-1-methoxy-5-oxo-1,2,3,5-tetrahydroindolizine-3-carboxylic acid

To a solution of (3S)-7-(6-amino-3-chloro-2-fluorophenyl)-1-methoxy-5-oxo-1,2,3,5-tetrahydroindolizine-3-carboxylic acid (400 mg, 1.14 mmol, 1.0 equiv.) in acetic acid (15 mL) were added trimethoxymethane (1.2 g, 11.36 mmol, 10.0 equiv.), and azidotrimethylsilane (1.3 g, 11.36 mmol, 10.0 equiv.). The resulting mixture was stirred overnight at room temperature, then concentrated under vacuum. The residue was partitioned between water and toluene. The organic layer was concentrated and the residue was purified by reverse column chromatography with CH₃CN/0.05% TFA water (5%→80%) to yield (3S)-7-(3-chloro-2-fluoro-6-(1H-tetrazol-1-yl)phenyl)-1-methoxy-5-oxo-1,2,3,5-tetrahydroindolizine-3-carboxylic acid as a light yellow solid.

### Step 7: 2-(4-((Methoxycarbonyl)amino)phenyl)-2-oxoethyl 7-(3-chloro-2-fluoro-6-(1H-tetrazol-1-yl)phenyl)-1-methoxy-5-oxo-1,2,3,5-tetrahydroindolizine-3-carboxylate

To a solution of 7-(3-chloro-2-fluoro-6-(1H-tetrazol-1-yl)phenyl)-1-methoxy-5-oxo-1,2,3,5-tetrahydroindolizine-3-carboxylic acid (400 mg, 0.99 mmol, 1.0 equiv.) in CH₃CN (8 mL) was added potassium carbonate (410 mg, 2.97 mmol, 3.0 equiv.) and the mixture was stirred at room temperature for 30 minutes. To the resulting solution was then added methyl (4-(2-bromoacetyl)phenyl)carbamate (400 mg, 1.48 mmol) in CH₃CN (2 mL), and the mixture was stirred at room temperature overnight and concentrated under reduced pressure. The residue was purified by silica gel column chromatography (0→5% CH₃OH/CH₂Cl₂) to yield 2-(4-((Methoxycarbonyl)amino)phenyl)-2-oxoethyl 7-(3-chloro-2-fluoro-6-(1H-tetrazol-1-yl)phenyl)-1-methoxy-5-oxo-1,2,3,5-tetrahydroindolizine-3-carboxylate as a light brown solid.

### Step 8: Methyl (4-(2-((1R,3S)-7-(3-chloro-2-fluoro-6-(1H-tetrazol-1-yl)phenyl)-1-methoxy-5-oxo-1,2,3,5-tetrahydroindolizin-3-yl)-1H-imidazol-5-yl)phenyl)carbamate

To a solution of 2-(4-(methoxycarbonylamino)phenyl)-2-oxoethyl 7-(3-chloro-2-fluoro-6-(1H-tetrazol-1-yl)phenyl)-1-methoxy-5-oxo-1,2,3,5-tetrahydroindolizine-3-carboxylate (400 mg, 0.67 mmol, 1.0 equiv.) in toluene (7 mL) were added ammonium acetate (516 mg, 6.70 mmol, 10.0 equiv.) and acetic acid (0.7 mL). The resulting mixture was heated at reflux for 2 h, quenched with H₂O (30 mL) and extracted with ethyl acetate (3×30 mL). The organic layers were combined, dried over Na₂SO₄, filtered, and concentrated under reduced pressure. The residue was purified by reverse phase chromatography on C18 (0→50% CH₃CN/H₂O) to yield methyl 4-(2-(7-(3-chloro-2-fluoro-6-(1H-tetrazol-1-yl)phenyl)-1-methoxy-5-oxo-1,2,3,5-tetrahydroindolizin-3-yl)-1H-imidazol-5-yl)phenylcarbamate as a white solid. The racemic mixture was separated by prep-chiral-HPLC separation. The collected fractions were combined and concentrated under vacuum to yield methyl (4-(2-((1R,3S)-7-(3-chloro-2-fluoro-6-(1H-tetrazol-1-yl)phenyl)-1-methoxy-5-oxo-1,2,3,5-tetrahydroindolizin-3-yl)-1H-imidazol-5-yl)phenyl)carbamate as a white solid.

LC/MS (ES, m/z): mass calculated for C₂₇H₂₂ClFN₈O₄: 576.14, measured: 577.30 [M+H]⁺. ¹H NMR (400 MHz, DMSO-d₆) δ 12.39 (d, J=164.3 Hz, 1H), 9.65 (d, J=38.6 Hz, 2H), 8.02 (t, J=8.2 Hz, 1H), 7.73 (d, J=8.7 Hz, 1H), 7.65-7.38 (m, 5H), 6.21 (s, 2H), 5.65 (d, J=8.6 Hz, 1H), 5.31 (t, J=7.7 Hz, 1H), 3.66 (s, 3H), 3.35-3.33 (m, 3H), 2.72 (dd, J=21.1, 11.9 Hz, 1H), 2.46-2.32 (m, 1H).

### Example 153: (*S)-6-(5-(6-Amino-2-fluoropyridin-3-yl)-1H-imidazol-2-yl)-2-(3-chloro-2-fluoro-6-(1H-tetrazol-1-yl)phenyl)-6,7,8,9-tetrahydro-4H-quinolizin-4-one

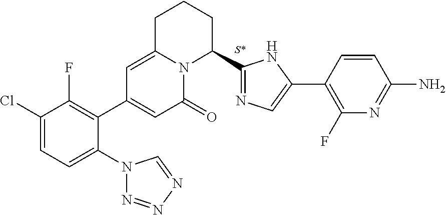 [SMILES: Nc1ccc(-c2cnc([C@@H]3CCCc4cc(-c5c(-n6cnnn6)ccc(Cl)c5F)cc(=O)n43)[nH]2)c(F)n1]

### Step 1: 4-(Benzyloxy)-2,6-dibromopyridine

To a solution of benzyl alcohol (17.2 mL, 166.25 mmol, 1.05 eq.) in 1,4-dioxane (167 mL) was added sodium hydride (6.6 g, 166.25 mmol, 1.05 eq. ca 60% dispersion in oil) at 0° C. The resulting mixture was stirred at room temperature for 1 h to generate a suspension of sodium phenyl methanolate.

To a solution of 2,4,6-tribromopyridine (50 g, 158.33 mmol, 1.0 eq.) in anhydrous N,N-dimethylformamide (800 mL) was added dropwise the above suspension of sodium phenyl methanolate at −20° C. The resulting mixture was warmed to room temperature slowly. After 2 h, the reaction was cooled to 0° C. and quenched with water. The mixture was filtered. The filter cake was dissolved in petroleum ether and stirred for 30 min. The filter cake was then collected, dried to yield the 4-(benzyloxy)-2,6-dibromopyridine as a white solid.

### Step 2: 4-(Benzyloxy)-2-bromo-6-((4-methoxybenzyl)oxy)pyridine

To a solution of (4-methoxyphenyl)methanol (17.6 g, 127.95 mmol, 1.05 eq.) in 1,4-dioxane (400 mL) was added sodium hydride (60%, 5.3 g, 118.43 mmol, 1.10 eq.) under nitrogen at 0° C. After 30 minutes, 4-(benzyloxy)-2,6-dibromopyridine (41.8 g, 121.86 mmol, 1.0 eq.) was added. The resulting mixture was stirred 80° C. for 2 h, then cooled to room temperature, diluted with water (200 mL), and extracted with ethyl acetate (3×200 mL). The organic layers were combined, dried over Na₂SO₄, filtered and concentrated to yield the 4-(benzyloxy)-2-bromo-6-((4-methoxybenzyl)oxy)pyridine as yellow oil. ¹H NMR (300 MHz, DMSO-d₆) δ 7.52-7.16 (m, 7H), 7.01-6.83 (m, 3H), 6.50 (d, J=1.9 Hz, 1H), 5.18 (d, J=4.9 Hz, 4H), 3.75 (s, 3H).

### Step 3: 4-(Benzyloxy)-6-bromopyridin-2-ol

To a solution of 4-(benzyloxy)-2-bromo-6-(4-methoxybenzyloxy)pyridine (60.5 g, 151.14 mmol) in dichloromethane (120 mL) was added trifluoroacetic acid (40 mL). The resulting mixture was stirred at room temperature for 3 hours. The solvent was removed under vacuum and the residue was suspended in sat. NaHCO₃ (aq., 100 mL). The suspension was stirred for 1 h. The solid was filtered and washed with water (200 mL) and petroleum ether (300 mL) to yield the 4-(benzyloxy)-6-bromopyridin-2-ol as white solid. LC/MS: mass calculated for C₁₂H₁₀BrNO₂: 278.99, measured: 280.0 [M+H]⁺.

### Step 4: Ethyl 2-(4-(benzyloxy)-6-bromo-2-oxopyridin-1(2H)-yl)acetate

To a solution of 6-bromo-4-methoxypyridin-2-ol (4 g, 14.28 mmol, 1 equiv) in DME/DMF (50 ml, 4/1) under N₂ was added NaH (602 mg, 15.05 mmol, 60%, 1.0 equiv) at 0° C. LiBr (2.5 g, 28.78 mmol, 2.016 equiv.) was added 10 min later. The mixture was stirred 20 min at room temperature, then ethyl 2-bromoacetate (4.8 g, 28.74 mmol, 2.0 equiv) was added. The mixture was stirred at 65° C. for 16 h and then poured into ice water. The resulting mixture was extracted with EtOAc. The organic layers were combined, washed with water, dried over Na₂SO₄, filtered and concentrated. The residue was purified by silica gel chromatography (0-40% PE/EA) to yield ethyl 2-(4-(benzyloxy)-6-bromo-2-oxopyridin-1(2H)-yl)acetate as a yellow solid. LC/MS: mass calculated for C₁₆H₁₆BrNO₄: 365.03, measured: 366.1 [M+H]⁺.

### Step 5: Ethyl 2-(4-(benzyloxy)-6-bromo-2-oxopyridin-1(2H)-yl)pent-4-enoate

To a solution of ethyl 2-(4-(benzyloxy)-6-bromo-2-oxopyridin-1(2H)-yl)acetate (3.3 g, 9.01 mmol, 1 equiv) in dry THF (30 ml) was added LiHDMS solution (10.8 mL, 10.8 mmol, 1.2 equiv, 1M) under nitrogen at −78° C. with stirring. The reaction mixture was stirred at −78° C. for 3h. To the resulting mixture was then added 3-iodoprop-1-ene (1.7 g, 10.12 mmol, 1.1 equiv) in THE (10 ml) at −78° C., and the reaction mixture was slowly warmed to room temperature and stirred for 16 h. The reaction was quenched with aq. NH₄Cl (100 mL), and the resulting mixture extracted with ethyl acetate (3×100 mL). The organic layers were combined, dried over Na₂SO₄, filtered and concentrated. The residue was purified by silica gel chromatography (0-40% PE/EA) to yield ethyl 2-(4-(benzyloxy)-6-bromo-2-oxopyridin-1(2H)-yl)pent-4-enoate as a yellow oil. LC/MS: mass calculated for C₁₉H₂₀BrNO₄: 405.06, measured: 405.8 [M+H]⁺.

### Step 6: Ethyl 2-(4-(benzyloxy)-2-oxo-6-vinylpyridin-1(2H)-yl)pent-4-enoate

To a solution of ethyl 2-[4-(benzyloxy)-6-ethenyl-2-oxo-1,2-dihydropyridin-1-yl]pent-4-enoate (1.1 g, 3.11 mmol, 1 equiv) in toluene (40 mL) was added Grubbs catalyst 2nd generation (529 mg, 0.62 mmol, 0.2 equiv) under N₂. The reaction mixture was stirred for 3 h at 80° C. After cooling to room temperature, the resulting mixture was concentrated and the residue was purified by silica gel chromatography (0-40% PE/EA) to yield ethyl 2-(4-(benzyloxy)-2-oxo-6-vinylpyridin-1(2H)-yl)pent-4-enoate as a yellow solid. LC/MS: mass calculated for C₁₉H₁₉NO₄: 325.13, measured: 326.1 M+H]⁺.

### Step 7: Ethyl 8-hydroxy-6-oxo-1,3,4,6-tetrahydro-2H-quinolizine-4-carboxylate

To a solution of ethyl 2-(4-(benzyloxy)-2-oxo-6-vinylpyridin-1(2H)-yl)pent-4-enoate (450 mg, 1.38 mmol, 1 equiv) in anhydrous MeOH (10 mL) at room temperature was added 10% Pd/C (100 mg, 0.94 mmol, 0.6 equiv). The resulting solution was stirred for 1 h at room temperature under hydrogen atmosphere. The solids were filtered out. The filtrate was concentrated and the residue was purified by silica gel chromatography (0-10% DCM/MeOH) to yield ethyl 8-hydroxy-6-oxo-1,3,4,6-tetrahydro-2H-quinolizine-4-carboxylate as a yellow solid. LC/MS: mass calculated for C₁₂H₁₅NO₄: 237.10, measured: 238.2 [M+H]⁺. ¹H NMR (400 MHz, DMSO-d₆) δ 10.49 (s, 1H), 5.73 (dt, J=2.6, 1.2 Hz, 1H), 5.47 (d, J=2.6 Hz, 1H), 4.93-4.87 (m, 1H), 4.00-4.09 (m, 2H), 2.80-2.61 (m, 2H), 2.01-2.10 (m, 2H), 1.73-1.43 (m, 2H), 1.17 (t, J=7.1 Hz, 3H).

### Step 8: Ethyl 6-oxo-8-(((trifluoromethyl)sulfonyl)oxy)-1,3,4,6-tetrahydro-2H-quinolizine-4-carboxylate

To a solution of ethyl 8-hydroxy-6-oxo-1,3,4,6-tetrahydro-2H-quinolizine-4-carboxylate (270 mg, 1.13 mmol, 1 equiv) in DMF (5 mL) at 0° C. was added TEA (0.23 g, 2.27 mmol, 2 equiv) and N-phenyl-bis(trifluoromethanesulfomimide) (488 mg, 1.36 mmol, 1.2 equiv). The mixture was stirred for 1 h at room temperature. The reaction mixture was quenched with water, and the resulting mixture extracted with ethyl acetate twice. The combined organic layers were combined, dried over Na₂SO₄, filtered and concentrated. The residue was purified by silica gel chromatography (0-20% PE/EA) to yield 6-oxo-8-(((trifluoromethyl)sulfonyl)oxy)-1,3,4,6-tetrahydro-2H-quinolizine-4-carboxylate as a yellow solid. LC/MS: mass calculated for C₁₃H₁₄F₃NO₆S: 369.05, measured: 370.0 [M+H]⁺.

### Step 9: Ethyl 8-(6-amino-3-chloro-2-fluorophenyl)-6-oxo-1,3,4,6-tetrahydro-2H-quinolizine-4-carboxylate

To a solution of 2,4-dichloropyrimidin-5-amine (340 mg, 0.92 mmol, 1.0 equiv) in 1,4-dioxane/H₂O (10 ml, 4/1) were added K₂CO₃ (381 mg, 2.75 mmol, 3 equiv), (6-amino-3-chloro-2-fluorophenyl)boronic acid (348 mg, 1.83 mmol, 2 equiv), and Pd(PPh₃)₄ (75 mg, 0.065 mmol, 0.1 equiv) under N₂. The reaction mixture was then stirred for 2 h at 90° C. The reaction was quenched with water. The resulting mixture was extracted with ethyl acetate. The organic layers were combined, dried over Na₂SO₄, filtered and concentrated. The residue was purified by silica gel chromatography (0-30% PE/EA) to yield ethyl 8-(6-amino-3-chloro-2-fluorophenyl)-6-oxo-2,3,4,6-tetrahydro-1H-quinolizine-4-carboxylate as a yellow solid. LC/MS: mass calculated for C₁₈H₁₈ClFN2O₃: 364.10, measured: 365.1 M+H]⁺.

### Step 10: 8-(6-Amino-3-chloro-2-fluorophenyl)-6-oxo-1,3,4,6-tetrahydro-2H-quinolizine-4-carboxylic acid

To a solution of ethyl 8-(6-amino-3-chloro-2-fluorophenyl)-6-oxo-2,3,4,6-tetrahydro-1H-quinolizine-4-carboxylate (320 mg, 0.87 mmol, 1 equiv) in EtOH (6 ml) at 0° C. was added NaOH (0.14 g, 3.50 mmol, 4 equiv). The mixture was stirred for 3 h at room temperature. The mixture solution was concentrated and diluted with water and adjusted to 5-6 with 2N HCl. The solid was filtered and dried under vacuum to yield 8-(6-amino-3-chloro-2-fluorophenyl)-6-oxo-2,3,4,6-tetrahydro-1H-quinolizine-4-carboxylic acid as a yellow solid. LC/MS: mass calculated for C₁₆H₁₄ClFN₂O₃: 336.07, measured: 337.1 [M+H]⁺.

### Step 11: 8-(3-Chloro-2-fluoro-6-(1H-tetrazol-1-yl)phenyl)-6-oxo-1,3,4,6-tetrahydro-2H-quinolizine-4-carboxylic acid

To a solution of 8-(6-amino-3-chloro-2-fluorophenyl)-6-oxo-2,3,4,6-tetrahydro-1H-quinolizine-4-carboxylic acid (300 mg, 0.89 mmol, 1 equiv) in AcOH (5 mL) were added trimethoxymethane (0.28 g, 2.67 mmol, 3 equiv) and TMSN₃ (0.31 g, 2.67 mmol, 3 equiv). The mixture was stirred for 2 h at 60° C. After cooling to room temperature, the reaction mixture was concentrated. The residue was purified by reverse phase chromatography on C18 column (0-70% H₂O/ACN) to yield 8-[3-chloro-2-fluoro-6-(1H-1,2,3,4-tetrazol-1-yl)phenyl]-6-oxo-2,3,4,6-tetrahydro-1H-quinolizine-4-carboxylic acid as a yellow solid. LC/MS: mass calculated for C₁₇H₁₃ClFN₅O₃:389.07, measured: 390.1 [M+H]⁺.

### Step 12: 2-(6-Acetamido-2-fluoropyridin-3-yl)-2-oxoethyl 8-(3-chloro-2-fluoro-6-(1H-tetrazol-1-yl)phenyl)-6-oxo-1,3,4,6-tetrahydro-2H-quinolizine-4-carboxylate

To a solution of 8-[3-chloro-2-fluoro-6-(1H-1,2,3,4-tetrazol-1-yl)phenyl]-6-oxo-2,3,4,6-tetrahydro-1H-quinolizine-4-carboxylic acid (0.23 g, 0.59 mmol, 1 equiv) in acetonitrile (8 ml) was added K₂CO₃ (0.16 g, 1.18 mmol, 2.0 equiv) at 0° C., followed by addition of N-[5-(2-bromoacetyl)-6-fluoropyridin-2-yl]acetamide (0.195 g, 0.70 mmol, 1.2 equiv). The mixture was stirred for 3 h at room temperature. The reaction was quenched with water. The resulting mixture was extracted with DCM. The organic layers were combined, dried over Na₂SO₄, filtered and concentrated. The residue was purified by silica gel chromatography (0-50% PE/EA) to yield 2-(6-acetamido-2-fluoropyridin-3-yl)-2-oxoethyl 8-[3-chloro-2-fluoro-6-(1H-1,2,3,4-tetrazol-1-yl)phenyl]-6-oxo-2,3,4,6-tetrahydro-1H-quinolizine-4-carboxylate as a yellow solid. LC/MS: mass calculated for C₂₆H₂₀ClF₂N₇O₅: 583.12, measured: 584.1 [M+H]⁺.

### Step 13: N-(5-(2-(8-(3-Chloro-2-fluoro-6-(1H-tetrazol-1-yl)phenyl)-6-oxo-1,3,4,6-tetrahydro-2H-quinolizin-4-yl)-1H-imidazol-5-yl)-6-fluoropyridin-2-yl)acetamide

To a solution of 2-(6-acetamido-2-fluoropyridin-3-yl)-2-oxoethyl 8-[3-chloro-2-fluoro-6-(1H-1,2,3,4-tetrazol-1-yl)phenyl]-6-oxo-2,3,4,6-tetrahydro-1H-quinolizine-4-carboxylate (270 mg, 0.46 mmol, 1 equiv) in toluene (7 mL) were added ammonium acetate (396 mg, 5.137 mmol, 11 equiv) and AcOH (0.7 mL). The reaction mixture was stirred overnight at 110° C. The reaction was quenched with aq. NaHCO₃. The resulting mixture was extracted with DCM/MeOH (10/1). The organic layers were combined, dried over Na₂SO₄, filtered and concentrated. The residue was purified by reverse phase chromatography on C18 column (0-60% H₂O/ACN) to yield N-(5-(2-(8-(3-Chloro-2-fluoro-6-(1H-tetrazol-1-yl)phenyl)-6-oxo-1,3,4,6-tetrahydro-2H-quinolizin-4-yl)-1H-imidazol-5-yl)-6-fluoropyridin-2-yl)acetamide as a yellow solid. LC/MS: mass calculated for C₂₆H₂₀ClF₂N₉O₂: 563.14, measured: 564.1 [M+H]⁺; ¹H NMR (300 MHz, DMSO-d₆) δ 9.85 (d, J=5.0 Hz, 1H), 8.69-8.80 (m, 1H), 7.95 (dd, J=8.7, 7.7 Hz, 1H), 7.65 (dd, J=8.7, 1.5 Hz, 1H), 7.36 (dd, J=5.3, 3.5 Hz, 1H), 6.99-7.18 (m, 2H), 6.81-6.96 (m, 1H), 5.52-5.72 (m, 1H), 3.97-4.30 (m, 2H), 3.83 (t, J=10.3 Hz, 1H), 2.65-2.98 (m, 3H), 2.23-2.48 (m, 2H), 1.95-2.19 (m, 2H).

### Step 14: (*S)-6-(5-(6-amino-2-fluoropyridin-3-yl)-1H-imidazol-2-yl)-2-(3-chloro-2-fluoro-6-(1H-tetrazol-1-yl)phenyl)-6,7,8,9-tetrahydro-4H-quinolizin-4-one

To a solution of N-[5-(2-{8-[3-chloro-2-fluoro-6-(1H-1,2,3,4-tetrazol-1-yl)phenyl]-6-oxo-2,3,4,6-tetrahydro-1H-quinolizin-4-yl}-1H-imidazol-5-yl)-6-fluoropyridin-2-yl]acetamide (70 mg, 0.124 mmol, 1 equiv) in THF (4 ml) was added 4N HCl (1 ml, 4 mmol). The reaction mixture was stirred for 2 h at 50° C. After cooling to room temperature, the reaction mixture was concentrated. The residue obtained was purified by reverse phase chromatography on C18 column (0-60% H₂O (0.5% TFA)/ACN) to yield 4-[5-(6-amino-2-fluoropyridin-3-yl)-1H-imidazol-2-yl]-8-[3-chloro-2-fluoro-6-(1H-1,2,3,4-tetrazol-1-yl)phenyl]-2,3,4,6-tetrahydro-1H-quinolizin-6-one as a light yellow solid. The light yellow solid was further purified by prep-chiral HPLC with the following conditions: column, CHIRALART Cellulose-SB2*25 cm, 5 um 107DA70007KSB99S05-2520WX; mobile phase, Phase A: MTBE-HPLC, Phase B: EtOH-HPLC; Total Run Time (min),15; Detector, UV 220/254 nm to yield (4R)-4-[5-(6-amino-2-fluoropyridin-3-yl)-1H-imidazol-2-yl]-8-[3-chloro-2-fluoro-6-(1H-1,2,3,4-tetrazol-1-yl)phenyl]-2,3,4,6-tetrahydro-1H-quinolizin-6-one as a light yellow solid.

LC/MS: mass calculated for C₂₄H₁₈ClF₂N₉O: 521.13, measured: 522.0 [M+H]⁺; ¹H NMR (300 MHz, DMSO-d₆) δ 12.04 (s, 1H), 9.64 (s, 1H), 7.99 (dd, J=8.7, 7.7 Hz, 1H), 7.86 (t, J=9.3 Hz, 1H), 7.73-7.60 (m, 1H), 7.12 (s, 1H), 6.41 (dd, J=8.2, 2.2 Hz, 3H), 6.11 (d, J=1.9 Hz, 1H), 5.98 (s, 1H), 5.83 (s, 1H), 2.89-2.61 (m, 2H), 2.32-1.94 (m, 2H), 1.66 (d, J=25.5 Hz, 2H). ¹⁹F NMR (300 MHz, DMSO-d₆) δ −70.95, −113.09

### Example 154: 6-(5-(6-Amino-2-fluoropyridin-3-yl)-1H-imidazol-2-yl)-2-(3-chloro-2-fluoro-6-(1H-tetrazol-1-yl)phenyl)-7,8,9,10-tetrahydropyrido[1,2-a]azepin-4(6H)-one

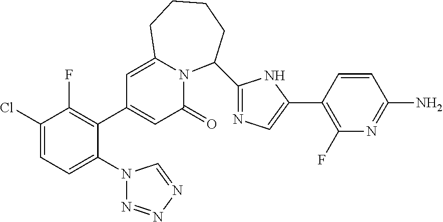 [SMILES: Nc1ccc(-c2cnc(C3CCCCc4cc(-c5c(-n6cnnn6)ccc(Cl)c5F)cc(=O)n43)[nH]2)c(F)n1]

### Step 1: 4-(benzyloxy)-2,6-dibromopyridine

To a solution of benzyl alcohol (17.2 mL, 166.25 mmol, 1.05 eq.) in 1,4-dioxane (167 mL) was added sodium hydride (6.6 g, 166.25 mmol, 1.05 eq. ca 60% dispersion in oil) at 0° C. The resulting mixture was stirred at room temperature for 1 h to yield a suspension of sodium phenylmethanolate.

To a solution of 2,4,6-tribromopyridine (50 g, 158.33 mmol, 1.0 eq.) in anhydrous N,N-dimethylformamide (800 mL) was added dropwise the above suspension of sodium phenylmethanolate at −20° C. The resulting mixture was warmed to room temperature slowly. After 2 h, the reaction was completed based on LCMS analysis. The reaction was cooled to 0° C. and quenched with water. The mixture was filtered. The filter cake was dissolved in petroleum ether and stirred for 30 min. The filter cake was collected, dried to yield the 4-(benzyloxy)-2,6-dibromopyridine as a white solid.

### Step 2: 4-(benzyloxy)-2-bromo-6-((4-methoxybenzyl)oxy)pyridine

To a solution of (4-methoxyphenyl)methanol (17.6 g, 127.95 mmol, 1.05 eq.) in 1,4-dioxane (400 mL) was added sodium hydride (60%, 5.3 g, 118.43 mmol, 1.10 eq.) under nitrogen at 0° C. After 30 minutes, 4-(benzyloxy)-2,6-dibromopyridine (41.8 g, 121.86 mmol, 1.0 eq.) was added. The resulting mixture was stirred 80° C. for 2 h, cooled to room temperature, diluted with water (200 mL), and the resulting mixture extracted with ethyl acetate (3×200 mL). The organic layers were combined, dried over Na₂SO₄, filtered and concentrated to yield the 4-(benzyloxy)-2-bromo-6-((4-methoxybenzyl)oxy)pyridine as a yellow oil. ¹H NMR (300 MHz, DMSO-d₆) δ 7.52-7.16 (m, 7H), 7.01-6.83 (m, 3H), 6.50 (d, J=1.9 Hz, 1H), 5.18 (d, J=4.9 Hz, 4H), 3.75 (s, 3H).

### Step 3: 4-(benzyloxy)-6-bromopyridin-2-ol

To a solution of 4-(benzyloxy)-2-bromo-6-(4-methoxybenzyloxy)pyridine (60.5 g, 151.14 mmol) in dichloromethane (120 mL) was added trifluoroacetic acid (40 mL). The resulting mixture was stirred at room temperature for 3 hours, concentrated, then diluted with sat. NaHCO₃ (aq., 100 mL). The resulting suspension was stirred for 1 h, the solid was filtered off and washed respectively with water (200 mL) and petroleum ether (300 mL) to yield the 4-(benzyloxy)-6-bromopyridin-2-ol as a white solid. LC/MS: mass calculated for C₁₂H₁₀BrNO₂: 278.99, measured: 280.0 [M+H]⁺.

### Step 4: ethyl 2-(4-(benzyloxy)-6-bromo-2-oxopyridin-1(2H)-yl)acetate

To a solution of 4-(benzyloxy)-6-bromopyridin-2-ol (39 g, 140.40 mmol) in N,N-dimethylformamide (50 mL) and 1,2-dimethoxyethane (250 mL) was added sodium hydride (60%, 5.9 g, 147.42 mmol, 1.05 eq.) under nitrogen at 0° C. After 1 h, lithium bromide (24.4 g, 280.811 mmol, 2.0 eq.) was added. The resulting mixture was stirred at room temperature for 1 hour, then cooled to 0° C. To the resulting mixture was then added ethyl 2-bromoacetate (31 mL, 280.81 mmol, 2.0 eq.). The resulting mixture was heated to 65° C. for 2 h under nitrogen. After cooling to 0° C., the mixture was poured into ice water (200 mL). The solid was filtered off and washed with ethyl acetate (75 mL) and petroleum ether (25 mL), and rinsed with water (50 mL) to yield the ethyl 2-(4-(benzyloxy)-6-bromo-2-oxopyridin-1(2H)-yl)acetate as an white solid. LC/MS: mass calculated for C₁₆H₁₆BrNO₄: 365.03, measured: 366.1 [M+H]⁺.

### Step 5: ethyl 2-(6-allyl-4-(benzyloxy)-2-oxopyridin-1(2H)-yl)acetate

Under N₂, to a solution of ethyl 2-(4-(benzyloxy)-6-bromo-2-oxopyridin-1(2H)-yl)acetate (2.4 g, 6.74 mmol, 1.0 eq) in toluene (50 mL) was added allyldibutyl(pentyl)stannane (2.7 g, 8.09 mmol, 1.2 eq) followed by addition of Pd(PPh₃)₄ (0.550 g, 0.67 mmol, 0.1 eq). The resulting mixture was heated at 100° C. for 2 h. The solvent was removed under reduced pressure and the residue was purified by silica gel chromatography (50% EtOAc/petroleum ether) to yield ethyl 2-(6-allyl-4-(benzyloxy)-2-oxopyridin-1(2H)-yl)acetate as a yellow solid. LC/MS: mass calculated for C₁₉H₂₁NO₄: 327.15, measured: 328.05 [M+H]⁺.

### Step 6: ethyl 2-(6-allyl-4-(benzyloxy)-2-oxopyridin-1(2H)-yl)pent-4-enoate

To a solution of ethyl 2-(6-allyl-4-(benzyloxy)-2-oxopyridin-1(2H)-yl)acetate (2.1 g, 6.42 mmol, 1.0 eq) in THE (50 mL) was added LiHMDS (7 mL, 7.00 mmol, 1.1 eq) at −78° C. The mixture was maintained with stirring for 1h. To the resulting mixture was then added ally iodide (1.1 g, 6.54 mmol, 1.05 eq). The reaction mixture was slowly warmed to room temperature and stirred for another 8 h. The reaction was quenched with NH₄Cl (aq. 50 mL), and the resulting mixture extracted with ethyl acetate (60 mL×5). The organic layers were combined, dried over Na₂SO₄, filtered and concentrated. The residue was purified by silica gel chromotography (60% EtOAc/petroleum ether) to yield ethyl 2-(6-allyl-4-(benzyloxy)-2-oxopyridin-1(2H)-yl)pent-4-enoate as yellow oil. LC/MS: mass calculated for C₂₂H₂₅NO₄: 367.18, measured: 368.20 [M+H]⁺.

### Step 7: (Z)-ethyl 2-(benzyloxy)-4-oxo-4,6,7,10-tetrahydropyrido[1,2-a]azepine-6-carboxylate

Under N₂, to a solution of ethyl 2-(6-allyl-4-(benzyloxy)-2-oxopyridin-1(2H)-yl)pent-4-enoate (670 mg, 1.82 mmol, 1.0 eq) in toluene (10 mL) was added Grubbs' 2nd catalyst (154 mg, 0.24 mmol, 0.1 eq). The resulting mixture was heated at 80° C. for 2 h and the solvent was removed under reduced pressure. The residue was purified by silica gel chromatography (50% EtOAc/petroleum ether) to yield (Z)-ethyl 2-(benzyloxy)-4-oxo-4,6,7,10-tetrahydropyrido[1,2-a]azepine-6-carboxylate as a brown oil. LC/MS: mass calculated for C₂₀H₂₁NO₄: 339.15, measured: 340.05 [M+H]⁺.

### Step 8: ethyl 2-hydroxy-4-oxo-4,6,7,8,9,10-hexahydropyrido[1,2-a]azepine-6-carboxylate

To a solution of (Z)-ethyl 2-(benzyloxy)-4-oxo-4,6,7,10-tetrahydropyrido[1,2-a]azepine-6-carboxylate (450 mg, 1.33 mmol, 1.0 eq) in MeOH (10 mL) was added 10% Pd/C (50 mg). The resulting mixture was stirred for 8 h under H₂. The catalyst was filtered off and the filtrate was concentrated under reduced pressure to yield ethyl 2-hydroxy-4-oxo-4,6,7,8,9,10-hexahydropyrido[1,2-a]azepine-6-carboxylate as purple solid. LC/MS: mass calculated for C₁₃H₁₇NO₄: 251.12, measured: 252.20 [M+H]⁺.

### Step 9: ethyl 4-oxo-2-(trifluoromethylsulfonyloxy)-4,6,7,8,9,10-hexahydropyrido[1,2-a]azepine-6-carboxylate

To a solution of ethyl 2-hydroxy-4-oxo-4,6,7,8,9,10-hexahydropyrido[1,2-a]azepine-6-carboxylate (270 mg, 1.07 mmol, 1.0 eq) and trifluoro-N-phenyl-N-(trifluoromethylsulfonyl)methane sulfonamide (767 mg, 2.15 mmol, 2.0 eq) in DMF (5 mL) was added triethylamine (271 mg, 2.68 mmol, 2.5 eq). The reaction mixture was stirred for 2 h, then quenched with H₂O (5 mL). The resulting mixture was extracted with ethyl acetate (10 mL×3). The organic layers were combined, dried over Na₂SO₄, filtered and concentrated under reduced pressure. The residue was purified by silica gel chromatography (EtOAc) to yield ethyl 4-oxo-2-(trifluoromethylsulfonyloxy)-4,6,7,8,9,10-hexahydropyrido[1,2-a]azepine-6-carboxylate as a yellow oil. LC/MS: mass calculated for C₁₄H₁₆F₃NO₆S: 383.07, measured: 384.00 [M+H]⁺.

### Step 10: yield ethyl 2-(6-amino-3-chloro-2-fluorophenyl)-4-oxo-4,6,7,8,9,10-hexahydropyrido[1,2-a]azepine-6-carboxylate

Under N₂, to a solution of ethyl 4-oxo-2-(trifluoromethylsulfonyloxy)-4,6,7,8,9,10-hexahydropyrido[1,2-a]azepine-6-carboxylate (280 mg, 0.730 mmol, 1.0 eq) in 1,4-dioxane/H₂O (8 mL/2 mL) were added 6-amino-3-chloro-2-fluorophenylboronic acid (276 mg, 1.46 mmol, 2.0 eq), Pd(PPh₃)₄ (84 mg, 0.073 mmol, 0.1 eq) and K₂CO₃ (201 mg, 1.46 mmol, 2.0 eq). The resulting mixture was heated at 90° C. for 2 h, quenched with H₂O (10 mL) and extracted with ethyl acetate (10 mL×3). The organic layers were combined, dried over Na₂SO₄, filtered and concentrated. The residue was purified by silica gel chromatography (50% EtOAc/petroleum ether) to yield ethyl 2-(6-amino-3-chloro-2-fluorophenyl)-4-oxo-4,6,7,8,9,10-hexahydropyrido[1,2-a]azepine-6-carboxylate as a yellow oil. LC/MS: mass calculated for C₁₉H₂₀ClFN₂O₃: 378.11, measured: 379.00[M+H]⁺.

### Step 11: 2-(6-amino-3-chloro-2-fluorophenyl)-4-oxo-4,6,7,8,9,10-hexahydropyrido[1,2-a]azepine-6-carboxylic acid

To a solution of ethyl 2-(6-amino-3-chloro-2-fluorophenyl)-4-oxo-4,6,7,8,9,10-hexahydropyrido[1,2-a]azepine-6-carboxylate (100 mg, 0.26 mmol, 1.0 eq) in THF/MeOH/H₂O (3 mL/1 mL/1 mL) was added lithium hydroxide (19 mg, 0.79 mmol, 3.0 eq). The resulting mixture was maintained for 2 h, then adjusted to pH 7 with aqueous HCl (1 N). The resulting mixture was extract with ethyl acetate (10 mL×3). The organic layers were combined, dried over Na₂SO₄, filtered and concentrated to yield 2-(6-amino-3-chloro-2-fluorophenyl)-4-oxo-4,6,7,8,9,10-hexahydropyrido[1,2-a]azepine-6-carboxylic acid as a yellow oil. LC/MS: mass calculated for C₁₇H₁₆ClFN₂O₃: 350.08, measured: 351.10 [M+H]⁺.

### Step 12: 2-(3-chloro-2-fluoro-6-(1H-tetrazol-1-yl)phenyl)-4-oxo-4,6,7,8,9,10-hexahydropyrido[1,2-a]azepine-6-carboxylic acid

To a mixture of 2-(6-amino-3-chloro-2-fluorophenyl)-4-oxo-4,6,7,8,9,10-hexahydropyrido[1,2-a]azepine-6-carboxylic acid (84 mg, 0.24 mmol, 1.0 eq), TMSN₃ (138 mg, 1.20 mmol, 5.0 eq), and trimethoxymethane (25 mg, 0.26 mmol, 10 eq) was added AcOH (5 mL). The mixture was stirred for 8 h at 60° C.

The solvent was removed under reduced pressure and the residue was purified by silica gel chromatography (100% EtOAc) to yield 2-(3-chloro-2-fluoro-6-(1H-tetrazol-1-yl)phenyl)-4-oxo-4,6,7,8,9,10-hexahydropyrido[1,2-a]azepine-6-carboxylic acid as a yellow oil. LC/MS: mass calculated for C₁₈H₁₅ClFN₅O₃: 403.08, measured: 404.05 [M+H]⁺.

### Step 13: 2-(6-acetamido-2-fluoropyridin-3-yl)-2-oxoethyl 2-(3-chloro-2-fluoro-6-(1H-tetrazol-1-yl)phenyl)-4-oxo-4,6,7,8,9,10-hexahydropyrido[1,2-a]azepine-6-carboxylate

To a solution of 2-(3-chloro-2-fluoro-6-(1H-tetrazol-1-yl)phenyl)-4-oxo-4,6,7,8,9,10-hexahydropyrido[1,2-a]azepine-6-carboxylic acid (80 mg, 0.19 mmol, 1.0 eq) in acetonitrile was added K₂CO₃ (109 mg, 0.79 mmol, 4.0 eq) followed by addition of N-(5-(2-bromoacetyl)-6-fluoropyridin-2-yl)acetamide (108 mg, 0.39 mmol, 2.0 eq). The mixture was stirred for 8 h. The solvent was removed under reduced pressure and the residue was purified by silica gel chromatography (30% EtOAc/petroleum ether) to yield 2-(6-acetamido-2-fluoropyridin-3-yl)-2-oxoethyl 2-(3-chloro-2-fluoro-6-(1H-tetrazol-1-yl)phenyl)-4-oxo-4,6,7,8,9,10-hexahydropyrido[1,2-a]azepine-6-carboxylate as a yellow oil. LC/MS: mass calculated for C₂₇H₂₂ClF₂N₇O₅: 597.13, measured: 598.15 [M+H]⁺.

### Step 14: N-(5-(2-(2-(3-chloro-2-fluoro-6-(1H-tetrazol-1-yl)phenyl)-4-oxo-4,6,7,8,9,10-hexahydropyrido[1,2-a]azepin-6-yl)-1H-imidazol-5-yl)-6-fluoropyridin-2-yl)acetamide

To a solution of 2-(6-acetamido-2-fluoropyridin-3-yl)-2-oxoethyl 2-(3-chloro-2-fluoro-6-(1H-tetrazol-1-yl)phenyl)-4-oxo-4,6,7,8,9,10-hexahydropyrido[1,2-a]azepine-6-carboxylate (120 mg, 0.201 mmol, 1.0 eq) in toluene/HOAc (2 mL/0.2 mL) was added NH₄OAc (154 mg, 2.00 mmol, 10 eq). The resulting mixture was heated at 80° C. for 3 h and concentrated under reduced pressure. The residue was purified by silica gel chromatography (30% EtOAc/petroleum ether) to yield N-(5-(2-(2-(3-chloro-2-fluoro-6-(1H-tetrazol-1-yl)phenyl)-4-oxo-4,6,7,8,9,10-hexahydropyrido[1,2-a]azepin-6-yl)-1H-imidazol-5-yl)-6-fluoropyridin-2-yl)acetamide as a yellow oil. LC/MS: mass calculated for C₂₅H₂₀ClF₂N₉O: 577.16, measured: 578.15 [M+H]⁺.

### Step 15: 6-(5-(6-amino-2-fluoropyridin-3-yl)-1H-imidazol-2-yl)-2-(3-chloro-2-fluoro-6-(1H-tetrazol-1-yl)phenyl)-7,8,9,10-tetrahydropyrido[1,2-a]azepin-4(6H)-one

To a solution of N-(5-(2-(2-(3-chloro-2-fluoro-6-(1H-tetrazol-1-yl)phenyl)-4-oxo-4,6,7,8,9,10-hexahydropyrido[1,2-a]azepin-6-yl)-1H-imidazol-5-yl)-6-fluoropyridin-2-yl)acetamide (95 mg, 0.16 mmol, 1.0 eq) in THE (2 mL) was added aqueous HCl (2 N) (0.5 mL). The resulting mixture was heated at 50° C. for 3 h and then concentrated under reduced pressure. The resulting residue was purified by prep-HPLC with the following conditions: Column, X Bridge C18, 19*150 mm, 5 μm; mobile phase. Mobile Phase A: (0.05% TFA), Mobile Phase B: ACN; Flow rate: 20 mL/min; Detector, 254 nm to yield 6-(5-(6-amino-2-fluoropyridin-3-yl)-1H-imidazol-2-yl)-2-(3-chloro-2-fluoro-6-(1H-tetrazol-1-yl)phenyl)-7,8,9,10-tetrahydropyrido[1,2-a]azepin-4(6H)-one as a yellow solid.

LC/MS: mass calculated for C₂₅H₂₀ClF₂N₉O: 535.14, measured: 536.25 [M+H]⁺. ¹H NMR (400 MHz, DMSO-d₆) δ 9.67 (s, 1H), 8.04 (t, J=8.2 Hz, 1H), 7.89 (dd, J=10.5, 8.2 Hz, 1H), 7.76 (dd, J=8.7, 1.5 Hz, 1H), 7.44 (s, 1H), 6.85 (dd, J=6.2, 2.8 Hz, 1H), 6.62 (br, 1H), 6.45 (dd, J=8.3, 2.0 Hz, 1H), 6.39 (d, J=1.9 Hz, 1H), 5.86 (d, J=2.1 Hz, 1H), 2.73-2.82 (m 1H), 2.55-2.71 (m, 1H), 2.22-2.31 (m 1H), 1.75-1.98 (m, 4H), 1.01-1.32 (m, 1H).

### Biological and Formulation Examples

### Biological Example 1: Factor XIa Inhibition Assay Utilizing a Fluorophore-Quencher Pair Peptide Substrate

A fluorescence intensity (FLINT) based assay was used to monitor inhibition of Factor XIa. The peptide substrate, 5Fam-KLTRAETV-K5Tamra (purchased from New England Peptide) was chosen based on the FXI sequence. Conversion of zymogen FXI to its activated form, FXIa, occurs by proteolytic cleavage by FXIa at two sites, Arg146 and Arg180. The custom peptide used in this assay was based on the Arg146 cleavage site of FXI. The peptide substrate was designed with a fluorophore-quencher pair, where the fluorescence is quenched until FXIa cleaves the 8-mer peptide after the Arg residue. The substrate K_{M} was fit to a substrate inhibition model whereby k_{cat}=0.86 s⁻¹, K_{M}=12.4 μM, Kᵢ=61.6 μM with an enzymatic efficiency, and k_{cat}/K_{M}=69523 M⁻¹s⁻¹.

The Factor XIa FLINT assay was used with the following 5Fam-KLTRAETV-K5Tamra assay buffer: 50 mM Tris, pH 7.5, 100 mM NaCl, 5 mM CaCl₂, 0.1 mg/mL BSA, 0.03% CHAPS. Assay buffer was prepared by mixing all ingredients fresh. 5Fam-KLTRAETV-K5Tamra peptide substrate was first prepared at 10 mM in 100% DMSO, then diluted to 3 mM in 100% DMSO. Assay buffer was then added directly to the 3 mM stock of substrate to prepare the 30 μM 2× working concentration (15 μM final concentration). The 2× Factor XIa stock solution was prepared by diluting 6.562 μM stock in 1× assay buffer for a 200 μM working stock solution (100 μM final concentration).

Test compound(s) were run in an 11-point, 3-fold serial dilution with a final top compound concentration of 100 nM. Final DMSO in assay was 2%. FXIa was preincubated with compound for 30-minutes and then substrate was added to initiate the reaction. The assay was run with kinetic (KIN) reads at 5 min intervals over 30 minutes. The time course was linear using 100 pM FXIa greater than 30 minutes. More specifically, the assay was run as follows:
100 nL of 0.01 mM test compound was dispensed into black 384-well non-binding Greiner BioOne 784900 plate for 0.1 μM final concentration;
5 μL of 1× assay buffer was dispensed to column 24 (low control) and 5 μL 2× Factor XIa solution was dispensed to columns 1-23 (column 23 high control);
the plate was centrifuged with a “cover” plate at 500 rpm for 1 min
the plate was pre-incubated for 30 minutes at room temperature with plate covered;
5 μL of 2×5Fam-KLTRAETV-K5Tamra peptide substrate was dispensed into the entire plate, columns 1-24;
the plate was centrifuged with a “cover” plate at 500 rpm for 1 min;
the plate was read monitoring fluorescence intensity on the BMG PHERAStar at room temperature, using fluorescence module 485 nm/520 nm.

Percent inhibition (IC₅₀) curves were generated per compound tested, and data was analyzed using a 4-parameter logistic fit using GeneData Screener. The relative fluorescence unit (RFU) values were normalized to percent inhibition using the following equation:

% inhibition=((HC−LC)−(compound−LC)/(HC−LC))*100

where LC−low control=mean signal of no Factor XIa or 100% inhibition of Factor XIa; HC−high control=mean signal of Factor XIa+5Fam-KLTRAETV-K5Tamra peptide substrate with DMSO only.

An 11-point dose response curve for the test compound(s) was generated using GENDATA to determine IC₅₀ value based on the following equation:

Y=Bottom+(Top−Bottom)/(1+10\hat{ }((log IC₅₀−X)*HillSlope))

where Y is the % inhibition in the presence of X inhibitor concentration, Top=high control=mean signal of Factor XIa+5Fam-KLTRAETV-K5Tamra peptide substrate with DMSO only; Bottom=low control−mean signal of no Factor XIa or 100% inhibition of Factor XIa; HillSlope−Hill coefficient; and IC50=concentration of compound with 50% inhibition in relation to top/high control.

### Biological Example 2: Kallikrein Inhibition Assay Utilizing a Quenched AMC Peptide Substrate

A fluorescence intensity (FLINT) based assay was used to monitor inhibition of human plasma kallikrein. The peptide substrate, Z-Gly-Pro-Arg-AMC (Purchased from Bachem; Catalog #I-1150) was chosen based on its relatively low K_{M} for kallikrein which enables running the assay at lower substrate concentrations to control background fluorescence. The kinetic parameters for this substrate were determined by fitting titration data to the Michaelis-Menten equation yielding a K_{M}=40 μM, k_{cat}=0.76 s⁻¹, and k_{cat}/K_{M}=18932 M⁻¹s⁻¹.

The Kallikrein FLINT assay was used with the following Z-Gly-Pro-Arg-AMC assay buffer: 50 mM Tris, pH 7.5, 100 mM NaCl, 5 mM CaCl₂, 0.1 mg/mL BSA, 0.03% CHAPS. Assay buffer was prepared by mixing all ingredients fresh. 2× Z-Gly-Pro-Arg-AMC peptide substrate was prepared by diluting 10 mM stock into 1× assay buffer for a 100 μM working concentration (50 μM final concentration). The 2× kallikrein stock solution was prepared by diluting 14.76 μM stock in 1× assay buffer for a 4 nM working stock solution (2 nM final concentration).

Test compound(s) were run in an 11-point, 3-fold serial dilution with a final top compound concentration of 1 μM. Final DMSO in assay was 2%. Plasma kallikrein was pre-incubated for 30-minute with compound and then 50 μM substrate was added to initiate the reaction. The assay was run with kinetic (KIN) reads at 5 min intervals over 30 minutes. The time course was linear using 2 nM kallikrein greater than 30 minutes. More specifically, the assay was run as follows:
100 nL of 0.1 mM test compound was dispensed into black 384-well non-binding Greiner BioOne 784900 plate for 1 μM final concentration;
5 μL of 1× assay buffer was dispensed to columns 24 (low control) and 5 μL 2× human kallikrein enzyme solution was dispensed to columns 1-23 (column 23 high control);
the plate was centrifuged with a “cover” plate at 500 rpm for 1 min
the plate was pre-incubated for 30 minutes at room temperature with plate covered;
5 μL of 2× Z-Gly-Pro-Arg-AMC peptide substrate was dispensed into the entire plate, columns 1-24;
the plate was centrifuged with a “cover” plate at 500 rpm for 1 min;
the plate was read monitoring fluorescence intensity on the BMG PHERAStar at room temperature, using fluorescence module 340 nm/440 nm.

Percent inhibition (IC₅₀) curves were generated per compound tested, and data was analyzed using a 4-parameter logistic fit using GeneData Screener. The relative fluorescence unit (RFU) values were normalized to percent inhibition using the following equation:

% inhibition=((HC−LC)−(compound−LC)/(HC−LC))*100

where LC—low control=mean signal of human kallikrein enzyme or 100% inhibition of human kallikrein enzyme; HC—high control=mean signal of Factor XIa+Z-Gly-Pro-Arg-AMC peptide substrate with DMSO only.

An 11-point dose response curve for the test compound(s) was generated using GENDATA to determine IC₅₀ value based on the following equation:

Y=Bottom+(Top−Bottom)/(1+10\hat{ }((log IC₅₀−X)*HillSlope))

where Y is the % inhibition in the presence of X inhibitor concentration, Top=high control=mean signal of human kallikrein enzyme+Z-Gly-Pro-Arg-AMC peptide substrate with DMSO only; Bottom=low control−mean signal of no human kallikrein enzyme or 100% inhibition of human kallikrein enzyme; HillSlope−Hill coefficient; and IC₅₀=concentration of compound with 50% inhibition in relation to top/high control.

Representative compounds of formula (I) of the present invention were tested according to the procedure described in Biological Example 1 and Biological Example 2 above, with results as listed in Table 6, below.

TABLE 6
Biological Activity, Representative Compounds of Formula (I)

| | | |
|---|---|---|
| | FXIa KIN | Kallikrein KIN |
| ID No. | Inh IC₅₀ (nM) | Inh IC₅₀ (nM) |

| 1 | 11.0 | | 47.0 | |
|---|---|---|---|---|
| 2 | 22.0 | | 170.0 | |
| 3 | 2.7 | | 39.2 | |
| 4 | >100 | | >1 | μM |
| 5 | >100 | | >1 | μM |
| 6 | 17.0 | | 72.8 | |
| 7 | 6.9 | | 38.0 | |
| 8 | >100 | | >1 | μM |
| 9 | 6.9 | | ~21.4 | |
| 10 | 18.3 | | 344.7 | |
| 11 | 2.2 | | 11.4 | |
| 12 | 63.7 | | 205.1 | |
| 13 | 12.7 | | 36.9 | |
| 14 | 188.8 | | 4.7 | μM |
| 15 | 164.6 | | 3.2 | μM |
| 16 | 78.1 | | 447.2 | |
| 17 | 890.2 | | 9.05 | uM |
| 18 | 9.1 | | 105.0 | |
| 19 | 25.4 | | 270.1 | |
| 20 | 19.7 | | 295.2 | |
| 21 | 64.4 | | 339.9 | |
| 22 | 252.9 | | 294.1 | |
| 23 | 5.0 | | 240.4 | |
| 24 | 1.1 | μM | >10 | μM |
| 25 | 0.6 | | 14.1 | |
| 26 | 22.6 | | 1.3 | μM |
| 27 | 483.3 | | 2.4 | μM |
| 28 | 311.4 | | 6.4 | μM |
| 29 | 0.5 | | 39.5 | |
| 30 | 872.6 | | 1.2 | μM |
| 31 | 250.2 | | 635.2 | |
| 32 | 11.2 | | 90.7 | |
| 33 | 6.8 | | 152.1 | |
| 34 | 0.1 | | 7.3 | |
| 35 | ~609.8 | | 9.8 | μM |
| 36 | 1.5 | μM | >10 | μM |
| 37 | 283.2 | | 7.8 | μM |
| 38 | 42.9 | | 538.9 | |
| 39 | 6.6 | μM | >10 | μM |
| 40 | 286.3 | | 6.4 | μM |
| 41 | 345.8 | | 2.2 | μM |
| 42 | 3.3 | | 33.0 | |
| 43 | 12.4 | | 107.7 | |
| 44 | 14.9 | | 97.8 | |
| 45 | >10 | μM | >10 | μM |
| 46 | 430.7 | | 487.6 | |
| 47 | 23.7 | | 21.7 | |
| 48 | 3.0 | μM | >10 | μM |
| 49 | 24.2 | | 44.3 | |
| 50 | 1.4 | | 16.0 | |
| 51 | 0.8 | | 49.2 | |
| 52 | 7.9 | μM | >10 | μM |
| 53 | 43.7 | | 54.0 | |
| 54 | 429.9 | | >10 | μM |
| 55 | 1.1 | | 42.5 | |
| 56 | 1.2 | μM | >10 | μM |
| 57 | 449.2 | | 3.4 | μM |
| 58 | 0.4 | | 3.1 | |
| 59 | 0.2 | | 11.6 | |
| 60 | 47.8 | | 184.7 | |
| 61 | >10 | μM | >10 | μM |
| 62 | 19.6 | | 18.1 | |
| 63 | 0.2 | | 3.5 | |
| 64 | 1.7 | μM | 5.4 | μM |
| 65 | 0.6 | | 1.3 | |
| 66 | 0.4 | | 3.6 | |
| 67 | 0.4 | | 20.6 | |
| 68 | >10 | μM | >10 | μM |
| 69 | 19.4 | | 118.2 | |
| 70 | 7.3 | | 42.9 | |
| 71 | >100 | | 735.7 | |
| 72 | 0.6 | | 3.3 | |
| 73 | 22.1 | | 54.8 | |
| 74 | 32.1 | | 27.7 | |
| 75 | 20.9 | | 97.6 | |
| 76 | 0.8 | | 12.1 | |
| 77 | 19.6 | | 166.8 | |
| 78 | >100 | | 462.7 | |
| 79 | 63.5 | | 323.5 | |
| 80 | 4.4 | | ~236.5 | |
| 81 | 0.6 | | 0.9 | |
| 82 | 53.8 | | 141.8 | |
| 83 | 56.2 | | 377.4 | |
| 84 | 5.7 | | 31.9 | |
| 85 | 52.0 | | >1 | μM |
| 86 | >100 | | 818.7 | |
| 87 | 1.5 | | 27.7 | |
| 88 | 2.6 | | 14.5 | |
| 89 | 5.1 | | ~45.9 | |
| 90 | 21.4 | | 298.1 | |
| 91 | 39.0 | | 167.8 | |
| 92 | 8.3 | | 15.6 | |
| 93 | 0.5 | | 20.8 | |
| 94 | 1.2 | | 12.0 | |
| 95 | 5.3 | | 71.7 | |
| 96 | >100 | | >1 | μM |
| 97 | 6.4 | μM | >10 | μM |
| 98 | 143.1 | | 134.8 | |
| 99 | 8.0 | | 23.8 | |
| 100 | 6.9 | | 7.6 | |
| 101 | 320.7 | | 1.3 | μM |
| 102 | 18.2 | | 446.1 | |
| 103 | 63.4 | | 151.3 | |
| 104 | 112.6 | | 115.2 | |
| 105 | >10 | μM | >10 | μM |
| 106 | 933.3 | | 6.4 | μM |
| 107 | 2.4 | | 95.9 | |
| 108 | 90.3 | | 7.1 | μM |
| 109 | >10 | μM | >10 | μM |
| 110 | 92.1 | | 2.3 | μM |
| 111 | 22.6 | | 145.5 | |
| 112 | 1.6 | | 7.8 | |
| 113 | 0.7 | | 16.8 | |
| 114 | 110.6 | | 283.8 | |
| 115 | 246.5 | | 6.0 | μM |
| 116 | 1.2 | | 26.8 | |
| 117 | 6.1 | μM | >10 | μM |
| 118 | 7.9 | | 15.0 | |
| 119 | 8.7 | | 286.6 | |
| 120 | 12.8 | | 11.2 | |
| 121 | 675.3 | | 4.8 | μM |
| 122 | 95.0 | | 5.6 | μM |
| 123 | 3.1 | | 17.5 | |
| 124 | 8.3 | | 52.9 | |
| 125 | 0.5 | | 3.0 | |
| 126 | 0.3 | | 3.4 | |
| 127 | 190.2 | | 723.4 | |
| 128 | 283.2 | | 746.8 | |
| 129 | 168.2 | | 1.6 | μM |
| 130 | >10 | μM | >10 | μM |
| 131 | 92.2 | | 669.3 | |
| 132 | >10 | μM | >10 | μM |
| 133 | 230.7 | | 4.0 | μM |
| 134 | 1.2 | | 16.8 | |
| 135 | 2.7 | μM | >10 | μM |
| 136 | 566.1 | | >10 | μM |
| 137 | 104.0 | | 7.2 | μM |
| 138 | 257.6 | | 4.3 | μM |
| 139 | 0.3 | | 18.0 | |
| 140 | ~575 | | 9.1 | μM |
| 141 | 0.5 | | 13.5 | |
| 142 | 0.2 | | 18.3 | |
| 143 | >10 | μM | >10 | μM |
| 144 | 62.1 | | 245.5 | |
| 145 | >10 | μM | >10 | μM |
| 146 | 9.0 | | 295.8 | |
| 147 | >10 | μM | >10 | μM |

| | | |
|---|---|---|
| 148 | 47.6 | 94.0 |
| 149 | 0.3 | 23.5 |
| 150 | 0.9 | 25.9 |
| 151 | 0.3 | 14.0 |
| 152 | 0.3 | 1.5 |
| Cmpd (III) | 1.2 | 100.0 |
| Cmpd (IV) | 48.0 | 160.0 |

### Formulation Example 1

### Solid, Oral Dosage Form—Prophetic Example

As a specific embodiment of an oral composition, 100 mg of Compound ID No. 34, prepared as described in Example #34, is formulated with sufficient finely divided lactose to provide a total amount of 580 to 590 mg to fill a size O hard gel capsule.

While the foregoing specification teaches the principles of the present invention, with examples provided for the purpose of illustration, it will be understood that the practice of the invention encompasses all of the usual variations, adaptations and/or modifications as come within the scope of the following claims and their equivalents.

Throughout this application, various publications are cited. The disclosure of these publications is hereby incorporated by reference into this application to describe more fully the state of the art to which this invention pertains.

1. A compound of formula (I) 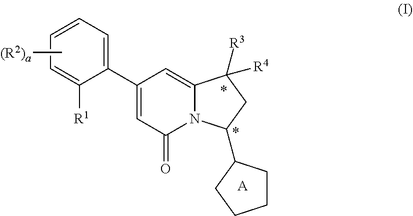
wherein
R¹ is selected from the group consisting of halogen, hydroxy, C₁₋₄alkyl, fluorinated C₁₋₄alkyl, C₁₋₄alkoxy, fluorinated C₁₋₄alkoxy, cyano, nitro, —NR^{A}R^{B}, —C(O)—C₁₋₄alkyl, C₃₋₆cycloalkyl, phenyl and 5 to 6 membered heterocyclyl;
wherein the C₃₋₆cycloalkyl, phenyl or 5 to 6 membered heterocyclyl is optionally substituted with one or more substituents independently selected from the group consisting of halogen, hydroxy, cyano, C₁₋₄alkyl, fluorinated C₁₋₄alkyl, C₁₋₄alkoxy, fluorinated C₁₋₄alkoxy, —C(O)OH, —C(O)O—(C₁₋₄alkyl), —NR^{A}R^{B}, —(C₁₋₄alkyl)-NR^{A}R^{B}, C₃₋₇cycloalkyl and 5 to 6 membered heterocyclyl; and wherein R^{A} and R^{B} are each independently selected from the group consisting of hydrogen and C₁₋₄alkyl;
a is an integer from 1 to 3;
each R² is independently selected from the group consisting of chloro, fluoro, methyl and methoxy;
R³ is hydrogen; and R⁴ is selected from the group consisting of methyl, methoxy, and ethoxy;
alternatively, R³ and R⁴ are taken together with the carbon atom to which they are bound to form cyclopropyl; 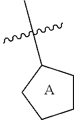
is selected from the group consisting of 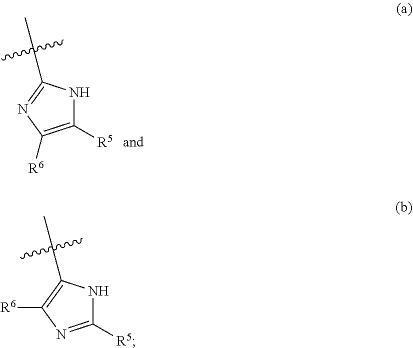
wherein R⁵ is selected from the group consisting of aryl and heterocyclyl;
wherein the aryl or heterocyclyl is optionally substituted with one or more substituents independently selected from the group consisting of halogen, hydroxy, oxo, C₁₋₄alkyl, fluorinated C₁₋₄alkyl, hydroxy substituted C₁₋₄alkyl, hydroxy substituted fluorinated C₁₋₄alkyl, C₁₋₄alkoxy, fluorinated C₁₋₄alkoxy, hydroxy substituted C₁₋₄alkoxy, hydroxy substituted fluorinated C₁₋₄alkoxy, cyano, —NR^{C}R^{D}, —(C₁₋₂alkylene)-NR^{C}R^{D}, —NR^{C}—C(O)—(C₁₋₄alkyl), —NR^{C}—C(O)—O—(C₁₋₄alkyl), —C(O)—NR^{C}—(C₁₋₄alkyl), —NR^{C}—SO₂—(C₁₋₄alkyl), cycloprop-1-yl, 1-hydroxy-cycloprop-1-yl, and —NR^{C}—C(O)-cyclopropyl; wherein R^{C} and R^{D} are each independently selected from the group consisting of hydrogen and C₁₋₄alkyl;
and R⁶ is selected from the group consisting of hydrogen, and halogen;
or a tautomer, stereoisomer, isotopologue, or pharmaceutically acceptable salt thereof.

2. The compound of claim 1, wherein
R¹ is selected from the group consisting of C₁₋₄alkyl, fluorinated C₁₋₂alkyl, C₁₋₂alkoxy, fluorinated C₁₋₂alkoxy, phenyl and 5 to 6 membered heterocyclyl;
wherein the phenyl or 5 to 6 membered heterocyclyl is optionally substituted with one to two substituents independently selected from the group consisting of halogen, cyano, C₁₋₄alkyl, fluorinated C₁₋₂alkyl, C₁₋₄alkoxy, and fluorinated C₁₋₂alkoxy;
a is an integer from 1 to 3;
each R² is independently selected from the group consisting of chloro, fluoro, and methyl;
R³ is hydrogen; and R⁴ is selected from the group consisting of methyl, methoxy, and ethoxy;
alternatively, R³ and R⁴ are taken together with the carbon atom to which they are bound to form cyclopropyl; 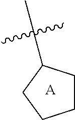
is selected from the group consisting of 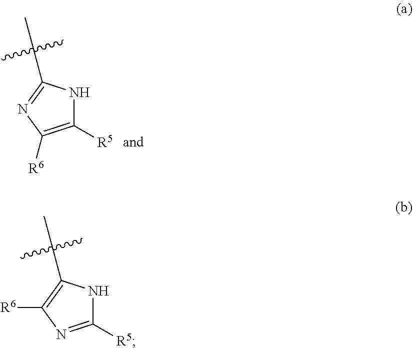
wherein R⁵ is selected from the group consisting of aryl, 5 to 6 membered heterocyclyl and 9 to 10 membered heterocyclyl;
wherein the aryl, 5 to 6 membered heterocyclyl or 9 to 10 membered heterocyclyl is optionally substituted with one or more substituents independently selected from the group consisting of halogen, hydroxy, oxo, C₁₋₄alkyl, fluorinated C₁₋₄alkyl, hydroxy substituted C₁₋₄alkyl, hydroxy substituted fluorinated C₁₋₄alkyl, C₁₋₄alkoxy, fluorinated C₁₋₄alkoxy, hydroxy substituted C₁₋₄alkoxy, hydroxy substituted fluorinated C₁₋₄alkoxy, cyano, —NR^{C}R^{D}, (C₁₋₂alkylene)-NR^{C}R^{D}, —NR^{C}—C(O)—(C₁₋₄alkyl), —NR^{C}—C(O)—O—(C₁₋₄alkyl), —C(O)—NR^{C}—(C₁₋₄alkyl), —NR^{C}—SO₂—(C₁₋₄alkyl), cycloprop-1-yl, 1-hydroxy-cycloprop-1-yl, and —NR^{C}—C(O)-cyclopropyl; wherein R^{C} and R^{D} are each independently selected from the group consisting of hydrogen and C₁₋₄alkyl;
and R⁶ is selected from the group consisting of hydrogen, and halogen;
or a tautomer, stereoisomer, isotopologue, or pharmaceutically acceptable salt thereof.

3. The compound of claim 1, wherein
R¹ is selected from the group consisting of 5 to 6 membered heterocyclyl; wherein the 5 to 6 membered heterocyclyl is optionally substituted with one to two substituents independently selected from the group consisting of halogen, and fluorinated C₁₋₂alkyl;
a is an integer from 1 to 2;
each R² is independently selected from the group consisting of chloro, and fluoro;
R³ is hydrogen; and R⁴ is selected from the group consisting of methyl, methoxy, and ethoxy;
alternatively, R³ and R⁴ are taken together with the carbon atom to which they are bound to form cyclopropyl; 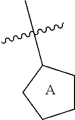
is selected from the group consisting of 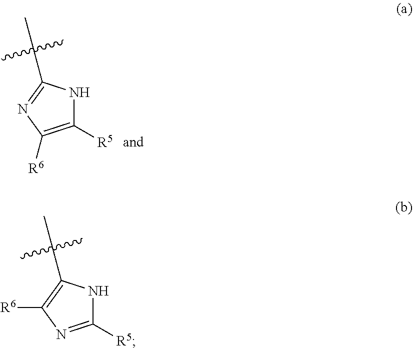
wherein R⁵ is selected from the group consisting of phenyl, 5 to 6 membered heteroaryl and 9 to 10 membered heterocyclyl;
wherein the phenyl, 5 to 6 membered heteroaryl or 9 to 10 membered heterocyclyl is optionally substituted with one to three substituents independently selected from the group consisting of halogen, oxo, C₁₋₄alkyl, fluorinated C₁₋₂alkyl, hydroxy substituted C₁₋₄alkyl, C₁₋₄alkoxy, hydroxy substituted C₁₋₄alkoxy, hydroxy substituted fluorinated C₁₋₄alkoxy, cyano, —NR^{C}R^{D}, —(C₁₋₂alkylene)-NR^{C}R^{D}, —NR^{C}—C(O)—(C₁₋₄alkyl), —NR^{C}—C(O)—O—(C₁₋₂alkyl), —C(O)—NR^{C}—(C₁₋₂alkyl), —NR^{C}—SO₂—(C₁₋₄alkyl), cycloprop-1-yl, 1-hydroxy-cycloprop-1-yl, and —NR^{C}—C(O)-cyclopropyl; wherein R^{C} and R^{D} are each independently selected from the group consisting of hydrogen and C₁₋₂alkyl;
and R⁶ is selected from the group consisting of hydrogen, and halogen;
or a tautomer, stereoisomer, isotopologue, or pharmaceutically acceptable salt thereof.

4. The compound of claim 1, wherein
R¹ is selected from the group consisting of 4-(trifluoro-methyl)-1,2,3-triazol-1-yl, and 1,2,3,4-tetrazol-1-yl;
n is an integer from 1 to 2;
each R² is independently selected from the group consisting of 2-fluoro, 3-chloro, and 4-chloro;
R³ is H; and R⁴ is selected from the group consisting of methyl, R*-methyl, S*-methyl, R-methyl, S-methyl, R*-methoxy, R-methoxy, and R-ethoxy;
alternatively, R³ and R⁴ are taken together with the carbon atom to which they are bound to form cyclopropyl; 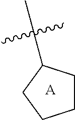
is selected from the group consisting of 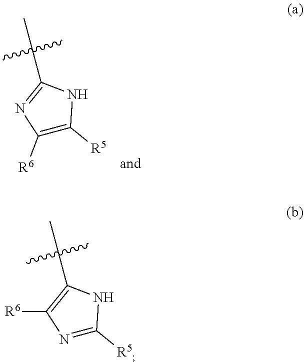
wherein R⁵ is selected from the group consisting of 4-(methoxy-carbonyl-amino)-phenyl, 4-(d3-methoxy-carbonyl-amino)-phenyl, 4-(methyl-sulfonyl-amino)-phenyl, pyrazol-4-yl, 1-methyl-pyrazol-5-yl, 1-methyl-4-cyano-pyrazol-5-yl, 1-methyl-3-amino-pyrazol-5-yl, 2-amino-thiazol-5-yl, 2-amino-4-methyl-thiazol-5-yl, 2-amino-4-(trifluoro-methyl)-thiazol-5-yl, 2-(methyl-carbonyl-amino)-thiazol-5-yl, 1,2,3-thiadiazol-5-yl, 1-methyl-1,2,3-triazol-1-yl, pyridin-3-yl-6-one, 6-amino-pyridin-3-yl, 2-fluoro-6-amino-pyridin-3-yl, 2-chloro-6-amino-pyridin-3-yl, 6-(methyl-amino-carbonyl)-pyridin-3-yl, 2-(2S*-(hydroxy)-3,3,3-trifluoro-n-propyloxy)-3-fluoro-pyridin-4-yl, 2-(2R*-(hydroxy)-3,3,3-trifluoro-n-propyloxy)-3-fluoro-pyridin-4-yl, 2-fluoro-pyridin-4-yl, 2-methyl-pyridin-4-yl, 2-cyano-pyridin-4-yl, 2-methoxy-pyridin-4-yl, 2-(trifluoro-methyl)-pyridin-4-yl, 2-(hydroxy-methyl)-3-fluoro-pyridin-4-yl, 2-(1-hydroxy-ethyl)-3-fluoro-pyridin-4-yl, 2-(methyl-amino-carbonyl)-pyridin-4-yl, 3-fluoro-6-amino-pyridin-4-yl, 2-cyano-3-methyl-pyridin-4-yl, 2-amino-3-fluoro-pyridin-4-yl, 2-(amino-methyl)-pyridin-4-yl, 2-(2-hydroxy-2-methyl-n-propyloxy)-3-fluoro-pyridin-4-yl, 2-(1-hydroxy-cycloprop-1-yl)-3-fluoro-pyridin-4-yl, 2-fluoro-3-amino-pyridin-5-yl, 2-amino-6-methyl-pyridin-5-yl, 2-amino-3-fluoro-pyridin-5-yl, 2-(methoxy-carbonyl-amino)-pyridin-5-yl, 2-(cyclopropyl-carbonyl-amino)-pyridin-5-yl, 2-amino-3-fluoro-6-methyl-pyridin-5-yl, 2-chloro-6-amino-pyrazin-3-yl, 2-fluoro-6-amino-pyrazin-3-yl, 2-amino-pyrazin-5-yl, 2-amino-6-(trifluoro-methyl)-pyrazin-5-yl, 2-amino-6-methoxy-pyrazin-5-yl, 2-amino-6-(difluoro-methyl)-pyrazin-5-yl, 2-amino-6-cyclopropyl-pyrazin-5-yl, 2-amino-6-methyl-pyrazin-5-yl, 6-amino-pyridazin-3-yl, 2-amino-pyrimidin-4-yl, 2-methoxy-pyrimidin-4-yl, 2-amino-pyrimidin-5-yl, indolin-5-yl-2-one, quinolin-7-yl-4(1H)-one, 2-methyl-indazol-5-yl, 3-amino-benzoisothiazol-6-yl, 3,4-dihydro-1,7-naphthyridin-6-yl-2(1H)-one, imidazo[1,2-a]pyridin-6-yl, and pyrazolo[1,5-a]pyridin-5-yl;
and R⁶ is selected from the group consisting of hydrogen and fluoro;
or a tautomer, stereoisomer, isotopologue, or pharmaceutically acceptable salt thereof.

5. The compound of claim 1, wherein
R¹ is 1,2,3,4-tetrazol-1-yl;
a is an integer from 1 to 2;
each R² is independently selected from the group consisting of 2-fluoro, 3-chloro and 4-chloro;
R³ is hydrogen; and R⁴ is selected from the group consisting of methyl, R*-methyl, S*-methyl, R-methyl, S-methyl, R*-methoxy, R-methoxy, and R-ethoxy;
alternatively, R³ and R⁴ are taken together with the carbon atom to which they are bound to form cyclopropyl; 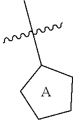
is selected from the group consisting of 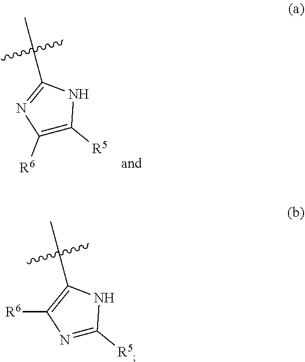
wherein R⁵ is selected from the group consisting of 4-(methoxy-carbonyl-amino)-phenyl, 4-(methyl-sulfonyl-amino)-phenyl, 1-methyl-4-cyano-pyrazol-5-yl, 1-methyl-3-amino-pyrazol-5-yl, 2-amino-thiazol-5-yl, 2-amino-4-methyl-thiazol-5-yl, 2-amino-4-(trifluoro-methyl)-thiazol-5-yl, 2-(methyl-carbonyl-amino)-thiazol-5-yl, 1-methyl-1,2,3-triazol-1-yl, pyridin-3-yl-6-one, 6-amino-pyridin-3-yl, 2-fluoro-6-amino-pyridin-3-yl, 2-chloro-6-amino-pyridin-3-yl, 6-(methyl-amino-carbonyl)-pyridin-3-yl, 2-fluoro-pyridin-4-yl, 2-methyl-pyridin-4-yl, 2-cyano-pyridin-4-yl, 2-(hydroxy-methyl)-3-fluoro-pyridin-4-yl, 2-(1-hydroxy-ethyl)-3-fluoro-pyridin-4-yl, 2-(methyl-amino-carbonyl)-pyridin-4-yl, 3-fluoro-6-amino-pyridin-4-yl, 2-cyano-3-methyl-pyridin-4-yl, 2-(amino-methyl)-pyridin-4-yl, 2-fluoro-3-amino-pyridin-5-yl, 2-amino-6-methyl-pyridin-5-yl, 2-amino-3-fluoro-pyridin-5-yl, 6-amino-pyridazin-3-yl, 2-amino-pyrimidin-4-yl, 2-methyl-indazol-5-yl, 3,4-dihydro-1,7-naphthyridin-6-yl-2(1H)-one, imidazo[1,2-a]pyridin-6-yl, and pyrazolo[1,5-a]pyridin-5-yl;
and R⁶ is selected from the group consisting of hydrogen and fluoro;
or a tautomer, stereoisomer, isotopologue, or pharmaceutically acceptable salt thereof.

6. The compound of claim 1, wherein
R¹ is 1,2,3,4-tetrazol-1-yl;
a is an integer from 1 to 2;
each R² is independently selected from the group consisting of 2-fluoro and 3-chloro;
R³ is hydrogen; and R⁴ is selected from the group consisting of R*-methyl, S*-methyl, R-methyl, S-methyl, R-methoxy, R*-methoxy and R-ethoxy;
alternatively, R³ and R⁴ are taken together with the carbon atom to which they are bound to form cyclopropyl; 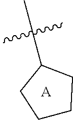
is 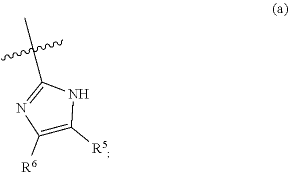
wherein R⁵ is selected from the group consisting of 4-(methoxy-carbonyl-amino)-phenyl, 4-(methyl-sulfonyl-amino)-phenyl, 1-methyl-4-cyano-pyrazol-5-yl, 1-methyl-3-amino-pyrazol-5-yl, 2-amino-thiazol-5-yl, 2-amino-4-methyl-thiazol-5-yl, 2-amino-4-(trifluoro-methyl)-thiazol-5-yl, 2-(methyl-carbonyl-amino)-thiazol-5-yl, 1-methyl-1,2,3-triazol-1-yl, pyridin-3-yl-6-one, 6-amino-pyridin-3-yl, 2-fluoro-6-amino-pyridin-3-yl, 2-chloro-6-amino-pyridin-3-yl, 6-(methyl-amino-carbonyl)-pyridin-3-yl, 2-fluoro-pyridin-4-yl, 2-methyl-pyridin-4-yl, 2-cyano-pyridin-4-yl, 2-(hydroxy-methyl)-3-fluoro-pyridin-4-yl, 2-(1-hydroxy-ethyl)-3-fluoro-pyridin-4-yl, 2-(methyl-amino-carbonyl)-pyridin-4-yl, 3-fluoro-6-amino-pyridin-4-yl, 2-cyano-3-methyl-pyridin-4-yl, 2-(amino-methyl)-pyridin-4-yl, 2-fluoro-3-amino-pyridin-5-yl, 2-amino-6-methyl-pyridin-5-yl, 2-amino-3-fluoro-pyridin-5-yl, 6-amino-pyridazin-3-yl, 2-amino-pyrimidin-4-yl, 2-methyl-indazol-5-yl, 3,4-dihydro-1,7-naphthyridin-6-yl-2(1H)-one, imidazo[1,2-a]pyridin-6-yl, and pyrazolo[1,5-a]pyridin-5-yl;
and R⁶ is selected from the group consisting of hydrogen and fluoro;
or a tautomer, stereoisomer, isotopologue, or pharmaceutically acceptable salt thereof.

7. The compound of claim 1, wherein
R¹ is 1,2,3,4-tetrazol-1-yl;
a is 2; the two R² are 2-fluoro and 3-chloro;
R³ is H; and R⁴ is selected from the group consisting of methyl, R*-methyl, S*-methyl, and S-methyl; 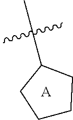
is 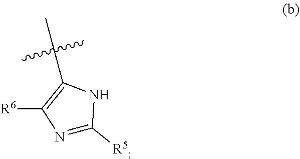
R⁵ is selected from the group consisting of 2-chloro-6-amino-pyrazin-3-yl, 2-(2S*-(hydroxy)-3,3,3-trifluoro-n-propyloxy)-3-fluoro-pyridin-4-yl, 2-(2R*-(hydroxy)-3,3,3-trifluoro-n-propyloxy)-3-fluoro-pyridin-4-yl, and 2-amino-pyrazin-5-yl;
R⁶ is selected from the group consisting of hydrogen and fluoro;
or a tautomer, stereoisomer, isotopologue, or pharmaceutically acceptable salt thereof.

8. The compound of claim 1, wherein
R¹ is 1,2,3,4-tetrazol-1-yl;
a is an integer from 1 to 2;
each R² is independently selected from the group consisting of 2-fluoro, and 3-chloro;
R³ is hydrogen; and R⁴ is selected from the group consisting of methyl, R*-methyl, S*-methyl, R-methyl, S-methyl, R*-methoxy, R-methoxy, and R-ethoxy;
alternatively, R³ and R⁴ are taken together with the carbon atom to which they are bound to form cyclopropyl; 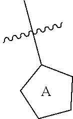
is selected from the group consisting of 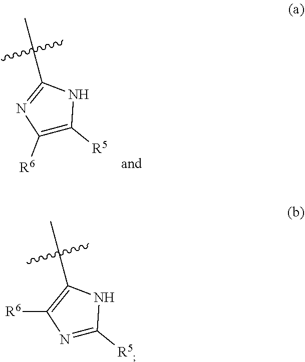
wherein R⁵ is selected from the group consisting of 4-(methoxy-carbonyl-amino)-phenyl, 4-(methyl-sulfonyl-amino)-phenyl, 2-amino-thiazol-5-yl, 2-amino-4-methyl-thiazol-5-yl, pyridin-3-yl-6-one, 2-fluoro-6-amino-pyridin-3-yl, 2-chloro-6-amino-pyridin-3-yl, 6-(methyl-amino-carbonyl)-pyridin-3-yl, 2-fluoro-pyridin-4-yl, 2-methyl-pyridin-4-yl, 2-cyano-pyridin-4-yl, 2-(hydroxy-methyl)-3-fluoro-pyridin-4-yl, 2-(1-hydroxy-ethyl)-3-fluoro-pyridin-4-yl, 2-(methyl-amino-carbonyl)-pyridin-4-yl, 3-fluoro-6-amino-pyridin-4-yl, 2-cyano-3-methyl-pyridin-4-yl, 2-(amino-methyl)-pyridin-4-yl, 2-fluoro-3-amino-pyridin-5-yl, 2-amino-6-methyl-pyridin-5-yl, 2-amino-3-fluoro-pyridin-5-yl, 6-amino-pyridazin-3-yl, 2-amino-pyrimidin-4-yl, 2-methyl-indazol-5-yl, 3,4-dihydro-1,7-naphthyridin-6-yl-2(1H)-one, imidazo[1,2-a]pyridin-6-yl, and pyrazolo[1,5-a]pyridin-5-yl;
and R⁶ is selected from the group consisting of hydrogen and fluoro;
or a tautomer, stereoisomer, isotopologue, or pharmaceutically acceptable salt thereof.

9. The compound of claim 1, wherein
R¹ is 1,2,3,4-tetrazol-1-yl;
a is an integer from 1 to 2;
each R² is independently selected from the group consisting of 2-fluoro, and 3-chloro;
R³ is hydrogen; and R⁴ is selected from the group consisting of R*-methyl, S*-methyl, R-methyl, S-methyl, R*-methoxy, R-methoxy, and R-ethoxy;
alternatively, R³ and R⁴ are taken together with the carbon atom to which they are bound to form cyclopropyl; 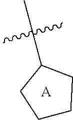
is selected from the group consisting of 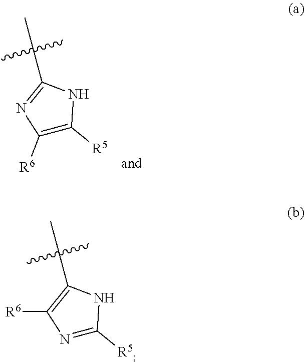
wherein R⁵ is selected from the group consisting of 4-(methoxy-carbonyl-amino)-phenyl, 4-(methyl-sulfonyl-amino)-phenyl, 2-amino-thiazol-5-yl, 2-fluoro-6-amino-pyridin-3-yl, 2-chloro-6-amino-pyridin-3-yl, 2-methyl-pyridin-4-yl, 2-(hydroxy-methyl)-3-fluoro-pyridin-4-yl, 2-cyano-3-methyl-pyridin-4-yl, 2-amino-6-methyl-pyridin-5-yl, and 3,4-dihydro-1,7-naphthyridin-6-yl-2(1H)-one;
and R⁶ is selected from the group consisting of hydrogen and fluoro;
or a tautomer, stereoisomer, isotopologue, or pharmaceutically acceptable salt thereof.

10. The compound of claim 1, wherein
R¹ is 1,2,3,4-tetrazol-1-yl;
a is an integer from 1 to 2;
each R² is independently selected from the group consisting of 2-fluoro, and 3-chloro;
R³ is hydrogen; and R⁴ is selected from the group consisting of R*-methyl, S*-methyl, R-methyl, S-methyl, R*-methoxy, R-methoxy, and R-ethoxy;
alternatively, R³ and R⁴ are taken together with the carbon atom to which they are bound to form cyclopropyl; 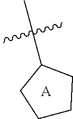
is selected from the group consisting of 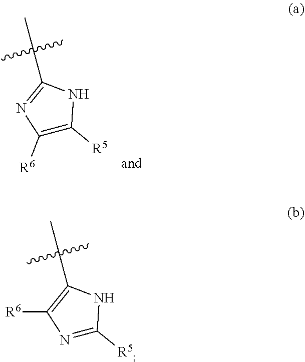
wherein R⁵ is selected from the group consisting of 4-(methoxy-carbonyl-amino)-phenyl, 2-fluoro-6-amino-pyridin-3-yl, 2-chloro-6-amino-pyridin-3-yl, and 2-amino-6-methyl-pyridin-5-yl;
and R⁶ is selected from the group consisting of hydrogen and fluoro;
or a tautomer, stereoisomer, isotopologue, or pharmaceutically acceptable salt thereof.

11. The compound of claim 1 selected from the group consisting of
Methyl(4-(2-((1S,3S)-7-(3-chloro-2-fluoro-6-(1H-tetrazol-1-yl)phenyl)-1-methyl-5-oxo-1,2,3,5-tetrahydroindolizin-3-yl)-1H-imidazol-5-yl)phenyl)carbamate;
(1S,3S)-3-(5-(6-Amino-2-chloropyridin-3-yl)-1H-imidazol-2-yl)-7-(3-chloro-2-fluoro-6-(1H-tetrazol-1-yl)phenyl)-1-methyl-2,3-dihydroindolizin-5(1H)-one;
Methyl (4-(2-((1R,3S)-7-(3-chloro-2-fluoro-6-(1H-tetrazol-1-yl)phenyl)-1-methoxy-5-oxo-1,2,3,5-tetrahydroindolizin-3-yl)-1H-imidazol-5-yl)phenyl)carbamate;
(1*R,3*R)-3-(5-(6-Amino-2-fluoropyridin-3-yl)-1H-imidazol-2-yl)-7-(3-chloro-2-fluoro-6-(1H-tetrazol-1-yl)phenyl)-1-methoxy-2,3-dihydroindolizin-5(1H)-one;
(1R,3S)-3-(5-(6-Amino-2-fluoropyridin-3-yl)-1H-imidazol-2-yl)-7-(3-chloro-2-fluoro-6-(1H-tetrazol-1-yl)phenyl)-1-ethoxy-2,3-dihydroindolizin-5(1H)-one;
and tautomers, stereoisomers, isotopologues and pharmaceutically acceptable salts thereof.

12. A compound selected from the group consisting of
(a) a compound of formula (III) 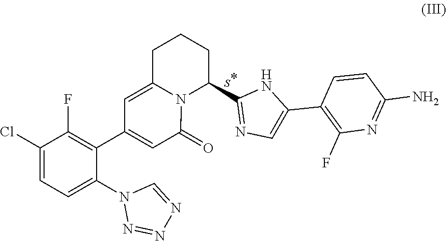 [SMILES: Nc1ccc(-c2cnc([C@@H]3CCCc4cc(-c5c(-n6cnnn6)ccc(Cl)c5F)cc(=O)n43)[nH]2)c(F)n1]
or a tautomer, stereoisomer, isotopologue, or pharmaceutically acceptable salt thereof; and
(b) a compound of formula (IV) 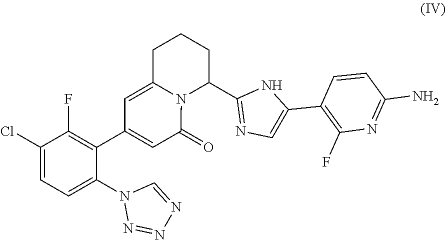 [SMILES: Nc1ccc(-c2cnc(C3CCCc4cc(-c5c(-n6cnnn6)ccc(Cl)c5F)cc(=O)n43)[nH]2)c(F)n1]
or a tautomer, stereoisomer, isotopologue, or pharmaceutically acceptable salt thereof.

13. A pharmaceutical composition comprising a pharmaceutically acceptable carrier and a compound of claim 1.

14. (canceled)

15. (canceled)

16. A method for the treatment of (a) a thromboembolic disorder; (b) an inflammatory disorder or a disorder; or (c) a disease or condition in which plasma kallikrein activity is implicated, comprising administering to a subject in need thereof a therapeutically effective amount of the compound of claim 1.

17. The method of claim 16, wherein the thromboembolic disorder is selected from the group consisting of arterial cardiovascular thromboembolic disorders, venous cardiovascular thromboembolic disorders, arterial cerebrovascular thromboembolic disorders, and venous cerebrovascular thromboembolic disorders.

18. The method of claim 16, wherein the thromboembolic disorder is selected from the group consisting of unstable angina, an acute coronary syndrome, atrial fibrillation, first myocardial infarction, recurrent myocardial infarction, ischemic sudden death, transient ischemic attack, stroke, atherosclerosis, peripheral occlusive arterial disease, venous thrombosis, deep vein thrombosis, thrombophlebitis, arterial embolism, coronary arterial thrombosis, cerebral arterial thrombosis, cerebral embolism, kidney embolism, pulmonary embolism, and thrombosis resulting from prosthetic valves or other implants, indwelling catheters, stents, cardiopulmonary bypass, hemodialysis, or other procedures in which blood is exposed to an artificial surface that promotes thrombosis.

19. The method of claim 16, wherein the thromboembolic disorder is selected from the group consisting of hereditary angioedema (HAE) and diabetic macular edema (DME).

20. The method of claim 16, wherein the inflammatory disorder is selected from the group consisting of sepsis, acute respiratory distress syndrome, and systemic inflammatory response syndrome.

21. The method of claim 16, wherein the disease or condition in which plasma kallikrein activity is implicated is selected from the group consisting of impaired visual acuity, diabetic retinopathy, diabetic macular edema, hereditary angioedema, diabetes, pancreatitis, nephropathy, cardiomyopathy, neuropathy, inflammatory bowel disease, arthritis, inflammation, septic shock, hypotension, cancer, adult respiratory distress syndrome, disseminated intravascular coagulation, and cardiopulmonary bypass surgery.

22. (canceled)

23. (canceled)

24. (canceled)

25. (canceled)

26. (canceled)

27. (canceled)

28. (canceled)

29. (canceled)

30. (canceled)

31. (canceled)
